(19) United States
(12) Patent Application Publication
(10) Pub. No.: US 2026/0071199 A1
(43) Pub. Date: Mar. 12, 2026

(54) COMPOSITION FOR MODIFYING A T CELL

(71) Applicant: JENTHERA THERAPEUTICS INC., Côte-Saint-Luc (CA)

(72) Inventor: Philip ROCHE, Montréal (CA)

(21) Appl. No.: 19/102,651

(22) PCT Filed: Aug. 9, 2023
(86) PCT No.: PCT/CA2023/051061

§ 371 Date: Feb. 10, 2025

Related U.S. Application Data
(60) Provisional application No. 63/397,150, filed on Aug. 11, 2022.

(51) Int. Cl. C12N 9/22; A61K 40/11; A61K 40/31; A61K 40/42; A61P 35/00; C12N 15/11; C12N 15/90

(52) U.S. Cl. CPC C12N 9/226; A61K 40/11; A61K 40/31; A61K 40/4211; A61P 35/00; C12N 15/11; C12N 15/907; C07K 2319/09; C12N 2310/20

(57) ABSTRACT

There is provided a composition for modifying a T cell, the composition comprising: a protein complex comprising a polynucleotide-modifying enzyme domain, a T cell membrane binding domain and an endosome escape domain; a guide oligonucleotide specific to a T cell receptor constant (TRAC) gene of the T cell; and a donor DNA comprising two homology arms at each end of the donor DNA homologous to exon1 of the TRAC gene and encoding therebetween a chimeric antigen T cell receptor comprising: translocation signal for translocation to a cell membrane of the T cell; a transmembrane domain; an intracellular signaling domain; and an extracellular antigen binding domain.

### CROSS REFERENCE TO A RELATED APPLICATION

This disclosure claims the priority of U.S. provisional application No. 63/397,150 filed on Aug. 11, 2022 and incorporated herein by reference in its entirety.

### TECHNICAL FIELD

This disclosure relates to the field of T cell genetic modifications, particularly the production of chimeric antigen receptor T cells (CAR-T) and vectors for performing the CAR gene editing.

### BACKGROUND OF THE ART

Chimeric antigen receptors (CARs) typically include an extracellular target-binding domain, a hinge region, a transmembrane domain that anchors the CAR to the cell membrane, and one or more intracellular domains that transmit activation signals, by signal cascade. The transmembrane domain is generally a hydrophobic helix that spans the thickness of the cell membrane. CARs are generally classified based on their number of costimulatory domains: first generation (CD3z only), second generation (one costimulatory domain and CD3z), and third generation (more than one costimulatory domain and CD3z). The purpose of introducing CAR molecules into a T cell is to redirect the T cell to a desired specificity and provide the necessary signals to drive full T cell activation. For example, a T cell can be genetically modified into a CAR-T cell that has specificity to an antigen presented by specific cancer cells. One particular area of clinical interest for CAR-T cells is the treatment of blood cancer types. The recognition of an antigen by a CAR-T cell is driven by the binding of the target-binding single-chain variable fragment (scFv) to surface antigens.

The hinge, which can also be referred to as a spacer, is the extracellular structural region of the CAR that separates the binding units from the transmembrane domain. The majority of CAR T cells are designed with immunoglobulin (Ig)-like domain hinges. These spacers generally supply stability for efficient CAR expression and activity. The hinge also provides flexibility to access the targeted antigen. The length of the hinge affects the binding efficiency of the CAR. For example, long spacers provide flexibility and therefore improved access to membrane-proximal epitopes or complex glycosylated antigens, whereas short spaces are more effective at binding distal epitopes. The length of the spacer provides adequate intercellular distance for immunological synapse formation. Accordingly, hinges can affect the overall performance of CAR-T cells.

Cellular therapies utilizing CAR-T cells are immunotherapeutic tools for combating conditions such as hematological diseases. Natural killer (NK) cells are a type of T cell that can also be modified into a CAR-T cell with a specific target. CD8 T cells, similarly to NK cells, have the capacity to target and kill a specific cell whereas CD4 T cells generally mediate the immune response to direct other killer cells. NK and CD8 T cells have the capacity to target and kill a cell based on the specificity of the CAR inserted therein. The use of CAR-T cells can greatly improve cancer treatment and patient outcomes. An allogeneic setting requires universal CAR T cells that can kill target tumor cells, avoid depletion by the host immune system, and proliferate without attacking host tissues. On the other hand an autologous therapy harvests the patient's own T cells that are then genetically modified into CAR-T cells and administered to the patient.

The modification of a T cell into a CAR-T cell requires a delivery means to provide the gene editing tools as well as the genetic material encoding the CAR. Viral vectors have been used for producing CAR-T cells. Examples of viral vectors include lentiviral vectors, gamma-retroviral vectors, recombinant adeno-associated virus vectors, and the like. In the case of retroviral transduction, the insertional mutagenesis has deleterious impact on the viability of primary T cells which is a major limitation for the application of retrovirus clinically. Lentivirus-based transduction represents a safer option because of a lower genotoxicity and insertional mutagenesis. Unfortunately, the efficiency of lentiviral transduction in primary T cells is low, often requiring multiple rounds of transduction, therefore also limiting its capacity in the clinical setting. Further disadvantages of retroviruses include the fact that they can only integrate into dividing cells in the mitosis stage and that the genetic integration is non-targeted. An additional disadvantage of lentiviral vectors is that they are non-integrative. In general, viral vectors present a mutagenesis risk which is a major concern in the clinical setting.

Electroporation has also been used to deliver the gene editing material and CAR construct to the T cells. However, electroporation leads to a high cell death rate and a difficult genomic integration. Overall, the electroporation technique has low efficacy and is not desirable in the clinical setting particularly when primary cells are involved. Moreover, the gene-transduction approaches usually lead to random integration of DNA into the target cell genome, resulting in the potential risk for off-target effects, such as the silencing of essential genes or tumour suppressor genes that may trigger cell apoptosis or malignant transformation.

Accordingly, improvements in transduction efficiency and targeting are desired particularly for primary cells. This would greatly improve the use of CAR-T cell therapies involving primary T cells for example in the treatment of cancer.

### SUMMARY

In one aspect, there is provided a composition for modifying a T cell, the composition comprising: a protein complex comprising a polynucleotide-modifying enzyme domain, a T cell membrane binding domain and an endosome escape domain; a guide oligonucleotide specific to a T cell receptor a constant (TRAC) gene of the T cell; and a donor DNA comprising two homology arms at each end of the donor DNA homologous to exon1 of the TRAC gene and encoding therebetween a chimeric antigen T cell receptor comprising: a translocation signal for translocation to a cell membrane of the T cell; a transmembrane domain; an intracellular signaling domain; and an extracellular antigen binding domain. In some embodiments, the protein complex further comprises a hapten binding domain, preferably the donor DNA is conjugated to a hapten and the hapten binds the hapten binding domain. In some embodiments, the protein complex further comprises a nuclear localisation sequence. In some embodiments, the chimeric antigen T cell receptor further comprises a CD8 hinge region. In some embodiments, the chimeric antigen T cell receptor further comprises a B cell lymphoma recognition domain. In some embodiments, the guide oligonucleotide is complementary to a sequence located between 250 nucleotides before the start codon of the exon 1 of the TRAC gene to 250 nucleotides after the start codon of the exon 1 of the TRAC gene. In some embodiments, the polynucleotide-modifying enzyme domain is covalently linked to the endosome escape domain. In some embodiments, the T cell membrane binding domain is a cationic peptide. In some embodiments, the T cell membrane binding domain is a cell recognition domain. In some embodiments, the cell recognition domain targets CD4, CD8, CD16 or CD56. In some embodiments, the cell recognition domain is covalently coupled to the endosome escape domain. In some embodiments, the cell recognition domain is a display domain being a peptidic recognition sequence of from 3 to 20 amino acids in length positioned in a loop or alpha helix on an external surface of the polynucleotide-modifying enzyme domain. In some embodiments, the peptidic recognition sequence is a complementarity-determining region (CDR). In some embodiments, the cell recognition domain is an antigen binding domain selected from Fab, single-domain antibody (sdAb), V_{HH}, or camelid antibody domain, positioned in a loop on an external surface of the polynucleotide-modifying enzyme. In some embodiments, the polynucleotide-modifying domain is a type II Cas, a functional analog thereof, a variant thereof or a derivative thereof. In some embodiments, the type II Cas is Cas9, a functional analog thereof, a variant thereof or a derivative thereof. In some embodiments, the polynucleotide-modifying domain is a type V Cas, a functional analog thereof, a variant thereof or a derivative thereof. In some embodiments, the extracellular antigen binding domain is specific to a cancer specific antigen.

In one aspect, the composition of the present disclosure is provided for use in cellular therapy, such as in the treatment of cancer.

In one aspect, there is provided the use of the composition of the present disclosure in cellular therapy, such as in the treatment of cancer.

In one aspect, there is provided a method of performing cellular therapy for a subject in need thereof, the method comprising providing ex vivo allogenic T cells, modifying the genome of the T cells with the composition of the present disclosure to obtain chimeric antigen receptor (CAR) T cells, and administering the CAR T cells to the subject.

In one aspect, there is provided a method of performing cellular therapy for a subject in need thereof, the method comprising providing ex vivo allogenic T cells, modifying the genome of the T cells with the composition of the present disclosure by having the composition bind to the cell membrane of the T cells and undergo cell internalization to obtain CAR-T cells, and administering the CAR T cells to the subject.

In one aspect, there is provided a method of treating cancer for a subject in need thereof, the method comprising providing allogenic T cells, modifying the genome of the T cells with the composition of the present disclosure to obtain CAR T cells, and administering the CAR T cells to the subject.

In one aspect, there is provided a method of performing cellular therapy for a subject in need thereof, the method comprising delivering the composition of the present disclosure to in vivo T cells of the subject to modify the genome of the T cells and obtain chimeric antigen receptor (CAR) T cells in vivo.

In one aspect, there is provided a method of treating cancer for a subject in need thereof, the method comprising delivering the composition of the present disclosure to in vivo T cells of the subject to modify the genome of the T cells and obtain chimeric antigen receptor (CAR) T cells in vivo.

In one aspect, there is provided a method of producing a CAR-T cell, the method comprising internalizing the composition of the present disclosure by binding to the cellular membrane of a T cell, and incubating the T cells to allow the composition to edit the genome of the T cell.

In one aspect, there is provided a polynucleotide-modifying enzyme comprising: a functional nuclease domain comprising a nuclease catalytic pocket; an antigen binding domain selected from Fab, single-domain antibody (sdAb), V_{HH}, or camelid antibody domain, in a loop that is positioned on an external surface of the polynucleotide-modifying enzyme, and said antigen binding domain recognizes a target cell receptor of a target cell to allow cell internalization of the polynucleotide-modifying enzyme in said target cell; and a linker of from 0 to 30 amino acids, upstream of the antigen binding domain. In some embodiments, the nanobody is a VHH. The linker sequence is preferably from 16 to 23 amino acids. The nuclease catalytic pocket is preferably a Cas nuclease catalytic pocket, recombinase catalytic pocket or a meganuclease catalytic pocket. The Cas can be a type II Cas such as cas9, a functional analog thereof, a variant thereof or a derivative thereof. In some embodiments, the nuclease catalytic pocket comprises a HNH nuclease domain. In some embodiments, the Cas is a type V Cas such as Cas12, a functional analog thereof, a variant thereof or a derivative thereof. In some embodiments, the Cas is a type VI Cas such as Cas13, a functional analog thereof, a variant thereof or a derivative thereof. In some embodiments, the Cas is a Cas14, a functional analog thereof, a variant thereof or a derivative thereof. In some embodiments, the nuclease catalytic pocket comprises a RuvC nuclease domain. In one aspect, there is provided a vector encoding the polynucleotide modifying enzyme comprising: a 5′ end and a 3′ end of a nuclease enzyme and in between the 5′ end and the 3′ end of the nuclease enzyme: an encoded functional nuclease domain coding the functional nuclease domain; an encoded antigen binding domain coding the antigen binding domain, the antigen binding domain; a linker sequence coding the linker, upstream of a 5′ end of the encoded antigen binding domain, coding the linker sequence.

Many further features and combinations thereof concerning the present improvements will appear to those skilled in the art following a reading of the instant disclosure.

### DESCRIPTION OF THE DRAWINGS

FIG. 1 is a schematic diagram of the delivery of a nuclease protein complex for editing a T cell.

FIG. 2 is a schematic diagram showing the mechanism of action of a receptor mediated delivery of a nuclease complex targeting the CD4 receptor of a T cell for intracellular delivery.

FIG. 3 is a map of a CAR construct.

FIG. 4 is a map of the vector of M7 Mav anti CD8.

FIG. 5 is a map of the vector of M7-Mav-anti CD4.

FIG. 6 is a map of the vector of C9mAur.

FIG. 7 is a map of the vector of C9mC4.

FIG. 8 is a map of the vector of C9C4 anti CD4n1.

FIG. 9A is an image of a gel electrophoresis showing the expression of the modified nuclease “Zero”.

FIG. 9B is a graph showing the expression of the modified nuclease “Zero” with the numbers labeled on the graph corresponding to the lane number of the gel of FIG. 9A.

FIG. 9C is an image of a gel electrophoresis showing the expression of the modified nuclease “L1”.

FIG. 9D is a graph showing the expression of the modified nuclease “L1” with the numbers labeled on the graph corresponding to the lane number of the gel of FIG. 9C.

FIG. 9E is an image of a gel electrophoresis showing the expression of the modified nuclease “L2”.

FIG. 9F is a graph showing the expression of the modified nuclease “L2” with the numbers labeled on the graph corresponding to the lane number of the gel of FIG. 9E.

FIG. 10A is a gel electrophoresis showing the cleaving activity of Zero, L1, and L2 on a 100 bp DNA template.

FIG. 10B is a graph showing the cleaving activity of Zero, L1, and L2 on a 100 bp DNA template over time.

FIG. 11 is an image of a gel electrophoresis of biotinylated CAR donor constructs and Biotin-CAR donors bound to Md7-MAV-CD47.

FIG. 12A is a bright field microscopy image of Jurkat cells gene edited with M7-Mav-CD4 (20× magnitude).

FIG. 12B is a fluorescence microscopy image for the green fluorescent protein showing Jurkat cells gene edited with M7-Mav-CD4 (GFP) (20× magnitude).

FIG. 13 is an image of a gel electrophoresis showing the CAR-DNA donors without C9mAur and biotinylated DNA donors complexed to C9mAur.

FIG. 14A is a fluorescence microscopy image of cells having received C9 only without donor DNA (i.e. control condition).

FIG. 14B is a fluorescence microscopy image of cells having receiving C9 with sgRNA1 guide and donor DNA.

FIG. 14C is a fluorescence microscopy image of cells having receiving C9 with sgRNA3 guide and donor DNA.

FIG. 14D is a fluorescence microscopy image of cells having receiving C9 with sgRNA11 guide and donor DNA.

FIG. 15 is an image of a gel electrophoresis for the polymerase chain reaction (PCR) with the insert confirmation primers (Jurkat cells triplicate samples J1, J2, and J3, with sgRNA11 as the guide RNA).

FIG. 16 is a graph of the raw read counts sequenced by next generation sequencing (NGS) with 3 biological replicates (samples 1, 2 and 3).

FIG. 17 is a graph of the percent reads sequenced by NGS with 3 biological replicates (samples 1, 2 and 3).

FIG. 18 is a spectra of the mass spectrometry of the RL peptide synthesized.

FIG. 19 is a graph of the high-performance liquid chromatography (HPLC) performed on the RL peptide synthesized.

FIG. 20 is a gel electrophoresis of the retardation assay showing that Zero, L1, L2 and L3 all bound the donor DNA (L3 is labeled as C9C4 on the gel, these labels are equivalent).

FIG. 21A is a fluorescent microscopy image showing Zero binding to the cell membrane of CD4+ primary T cells.

FIG. 21B is a fluorescent microscopy image showing L1 binding to the cell membrane of CD4+ primary T cells.

FIG. 21C is a fluorescent microscopy image showing L2 binding to the cell membrane of CD4+ primary T cells.

FIG. 21D is a fluorescent microscopy image showing L2 binding to the cell membrane of CD4+ primary T cells.

FIG. 22A is a flow cytometry graph showing the events (×1000) in function of TAMRA detection for the control condition (no nuclease, one hour incubation) on Jurkat CD4+ T-cells.

FIG. 22B is a flow cytometry graph showing the events (×1000) in function of TAMRA detection on Jurkat CD4+ T-cells one hour after receiving Zero.

FIG. 22C is a flow cytometry graph showing the events (×1000) in function of TAMRA detection on Jurkat CD4+ T-cells one hour after receiving L1.

FIG. 22D is a flow cytometry graph showing the events (×1000) in function of TAMRA detection on Jurkat CD4+ T-cells one hour after receiving L2.

FIG. 22E is a flow cytometry graph showing the events (×1000) in function of TAMRA detection on Jurkat CD4+ T-cells one hour after receiving L3.

FIG. 23 is a graph showing the fluorescence intensity count of green fluorescent protein (GFP) in cells treated for L1 overtime.

FIG. 24A is a flow cytometry graph showing the events (×1000) in function of TAMRA detection for the control condition (no nuclease, one hour incubation) on human primary CD4+ T-cells.

FIG. 24B is a flow cytometry graph showing the events (×1000) in function of TAMRA detection on human primary CD4+ T-cells one hour after receiving L1.

FIG. 24C is a flow cytometry graph showing the events (×1000) in function of TAMRA detection on human primary CD4+ T-cells one hour after receiving L2.

FIG. 24D is a flow cytometry graph showing the events (×1000) in function of TAMRA detection on human primary CD4+ T-cells one hour after receiving L3.

FIG. 25A is a flow cytometry graph showing the detection of TAMRA in function of GFP in the control condition (no nuclease provided in the Jurkat T-cell incubation of 48 hours).

FIG. 25B is a flow cytometry graph showing the events (×1000) in function of TAMRA detection in the control condition (no nuclease provided in the Jurkat T-cell incubation of 48 hours).

FIG. 25C is a flow cytometry graph showing the events (×1000) in function of GFP detection in the control condition (no nuclease provided in the Jurkat T-cell incubation of 48 hours).

FIG. 26A is a flow cytometry graph showing the detection of TAMRA in function of GFP in the control condition (Zero in a concentration of 0.33 ng/μL in the Jurkat T-cell incubation of 48 hours).

FIG. 26B is a flow cytometry graph showing the events (×1000) in function of TAMRA detection in the control condition (Zero in a concentration of 0.33 ng/μL in the Jurkat T-cell incubation of 48 hours).

FIG. 26C is a flow cytometry graph showing the events (×1000) in function of GFP detection in the control condition (Zero in a concentration of 0.33 ng/μL in the Jurkat T-cell incubation of 48 hours).

FIG. 26D is a flow cytometry graph showing the detection of TAMRA in function of GFP in the control condition (Zero in a concentration of 16 ng/μL in the Jurkat T-cell incubation of 48 hours).

FIG. 26E is a flow cytometry graph showing the events (×1000) in function of TAMRA detection in the control condition (Zero in a concentration of 16 ng/μL in the Jurkat T-cell incubation of 48 hours).

FIG. 26F is a flow cytometry graph showing the events (×1000) in function of GFP detection in the control condition (Zero in a concentration of 16 ng/μL in the Jurkat T-cell incubation of 48 hours).

FIG. 26G is a flow cytometry graph showing the detection of TAMRA in function of GFP in the control condition (Zero in a concentration of 33.3 ng/μL in the Jurkat T-cell incubation of 48 hours).

FIG. 26H is a flow cytometry graph showing the events (×1000) in function of TAMRA detection in the control condition (Zero in a concentration of 33.3 ng/μL in the Jurkat T-cell incubation of 48 hours).

FIG. 261 is a flow cytometry graph showing the events (×1000) in function of GFP detection in the control condition (Zero in a concentration of 33.3 ng/μL in the Jurkat T-cell incubation of 48 hours).

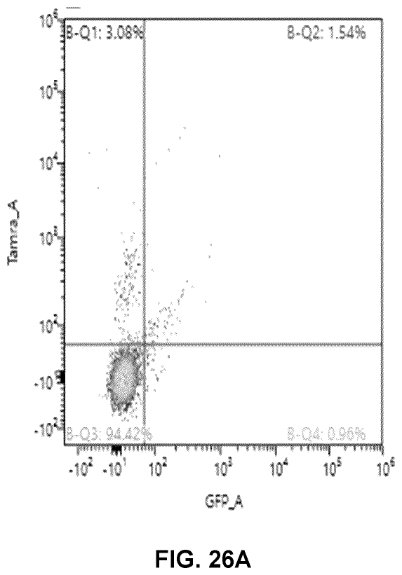

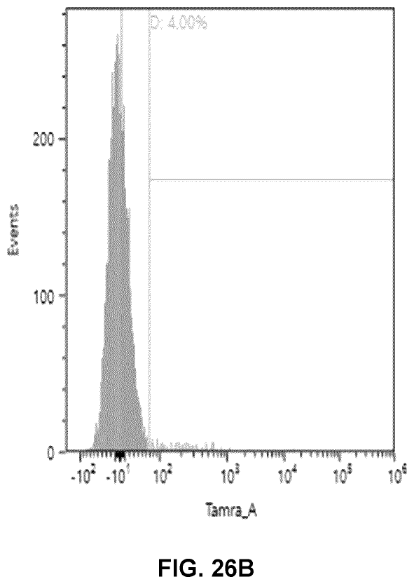

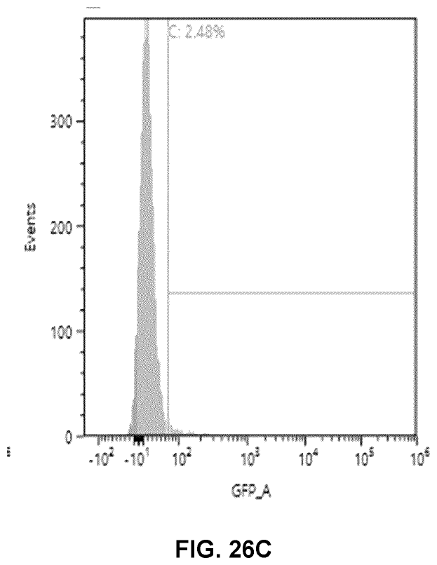

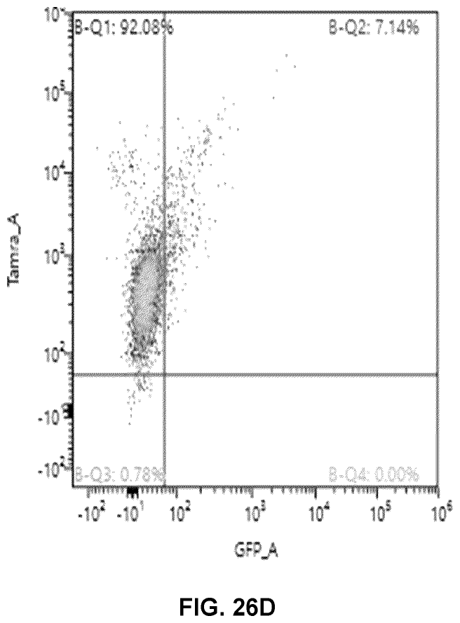

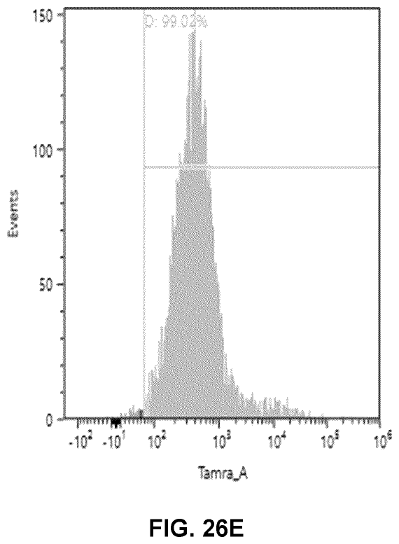

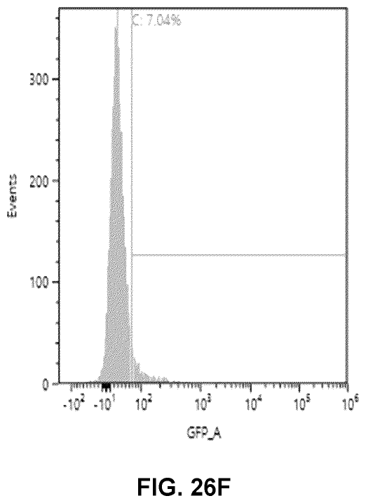

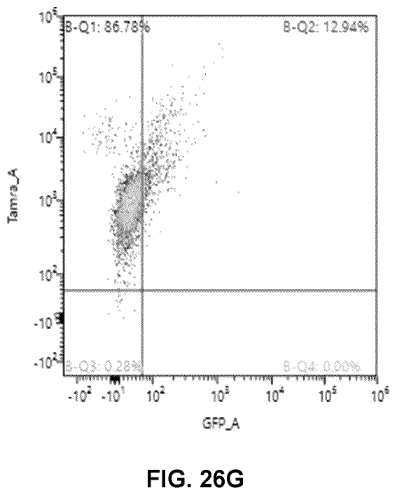

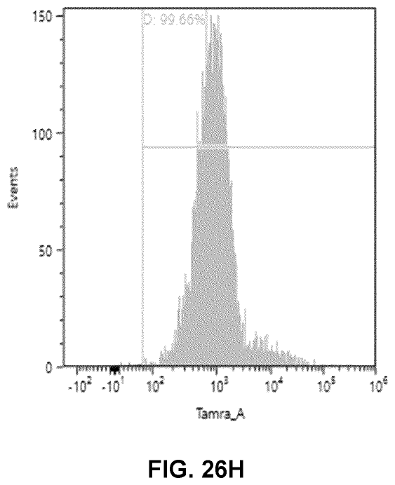

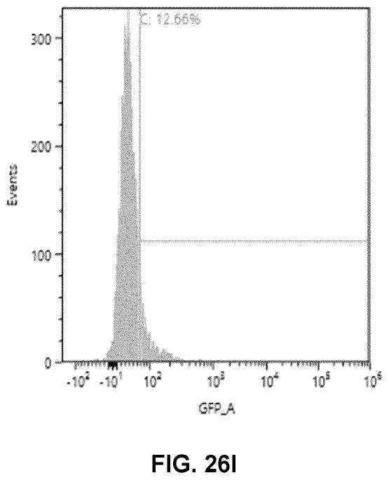

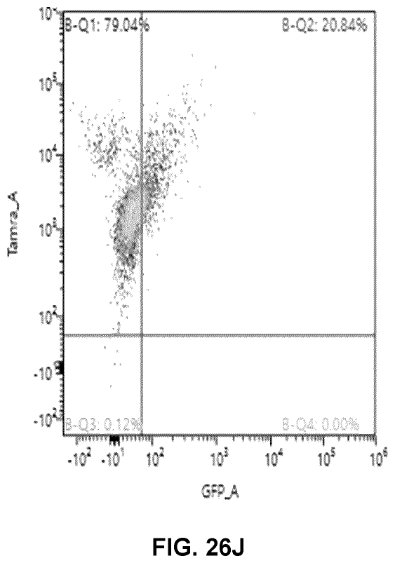

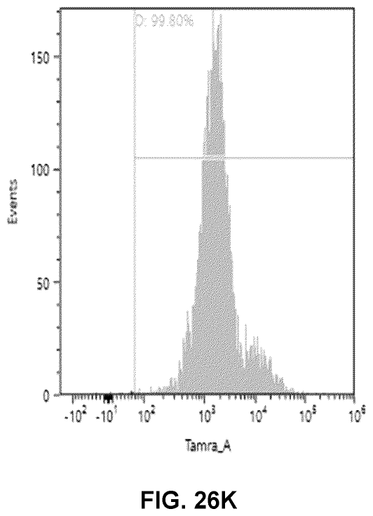

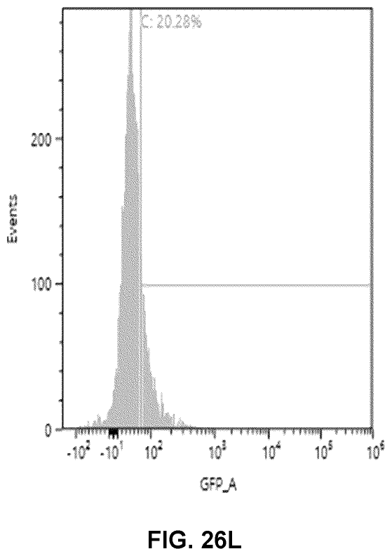

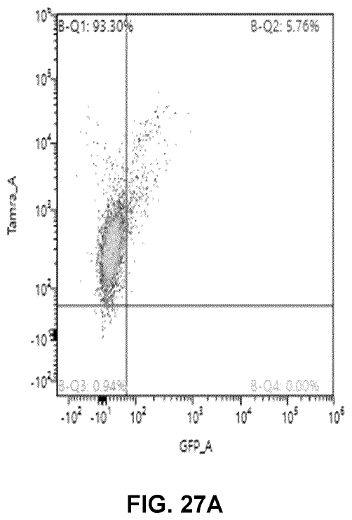

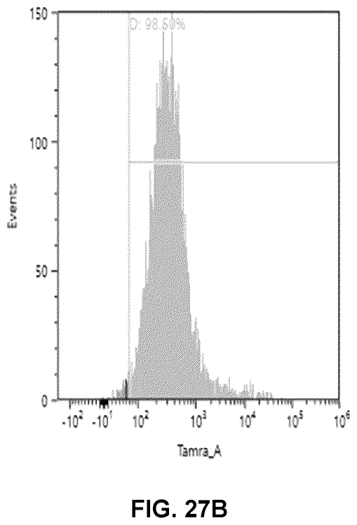

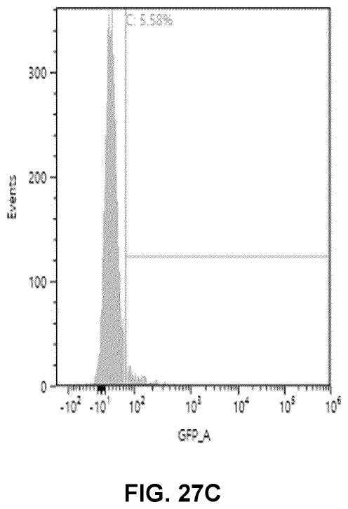

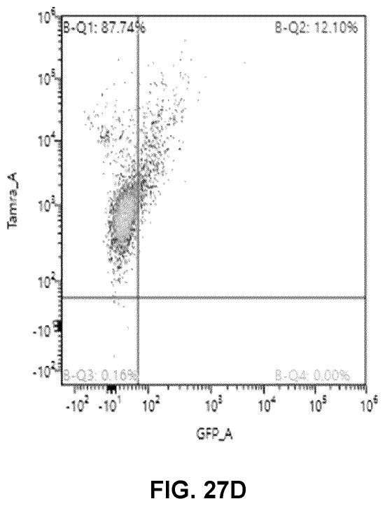

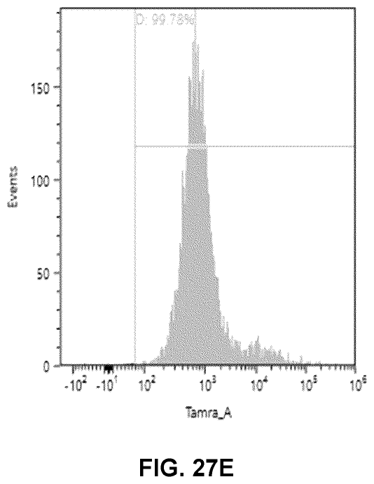

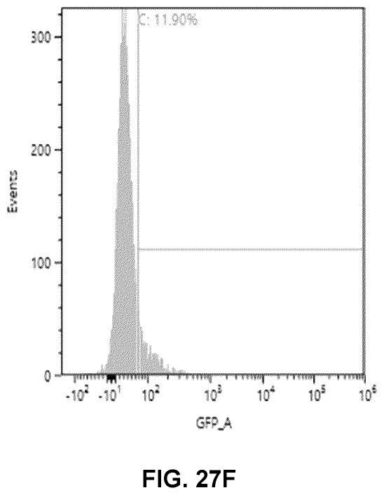

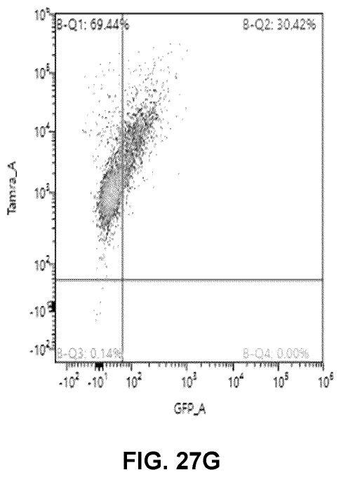

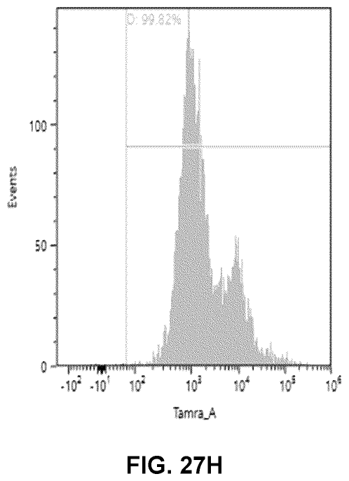

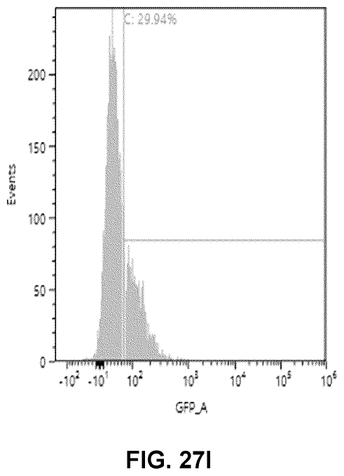

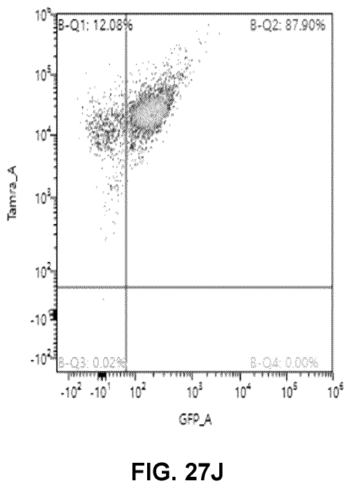

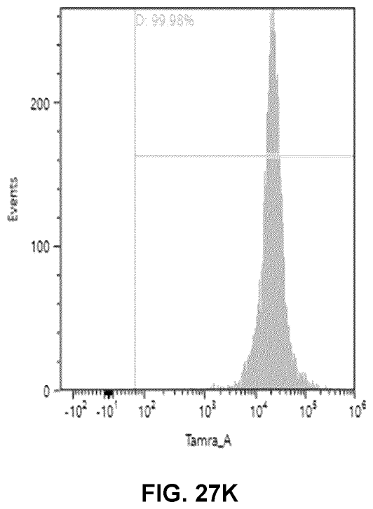

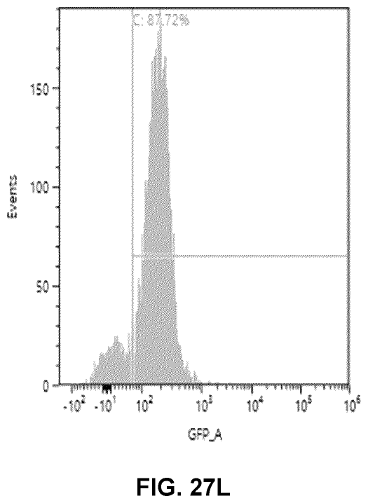

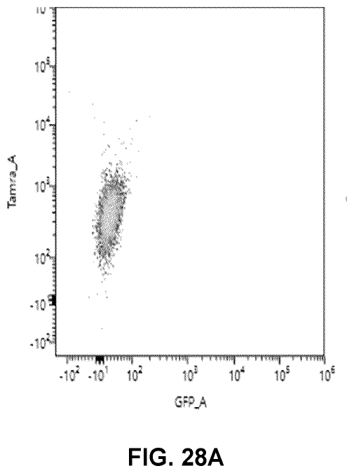

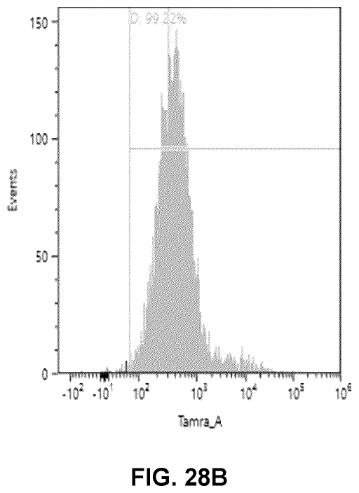

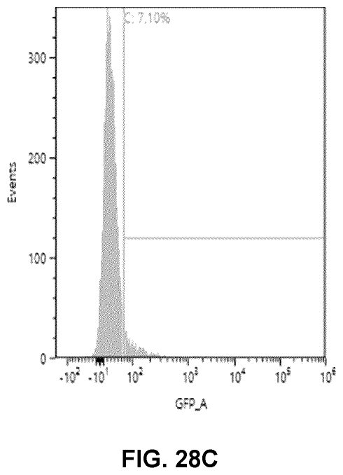

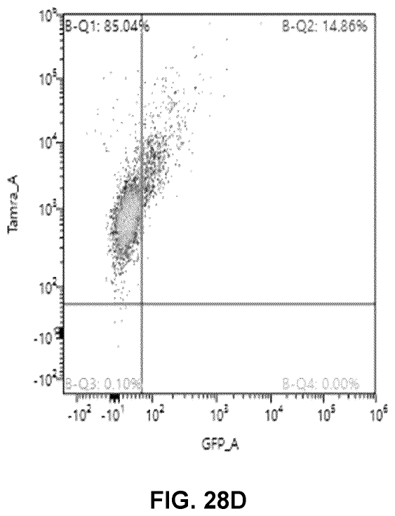

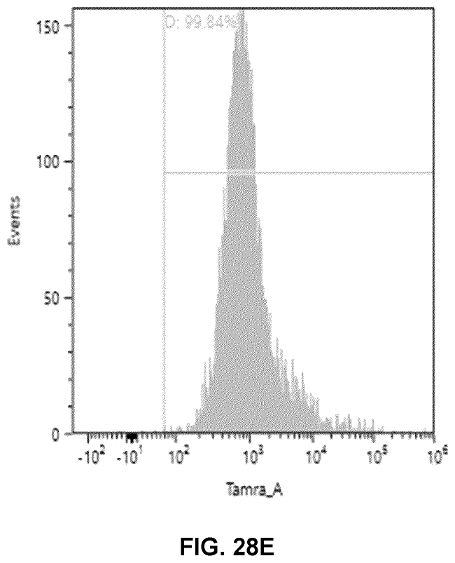

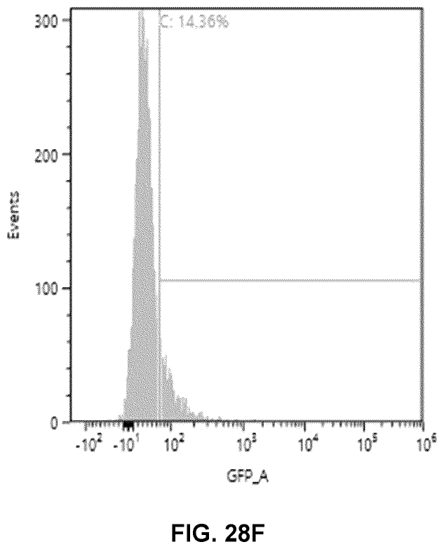

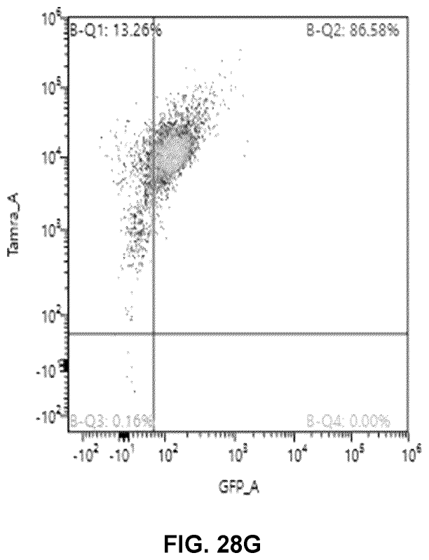

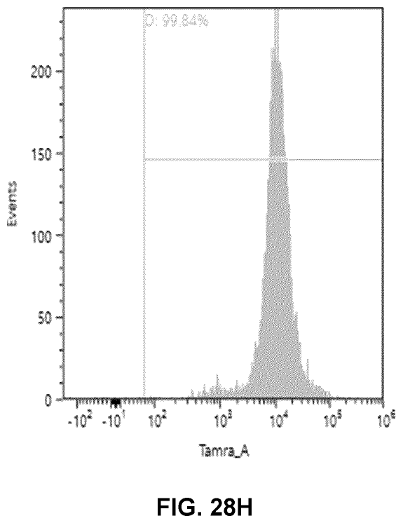

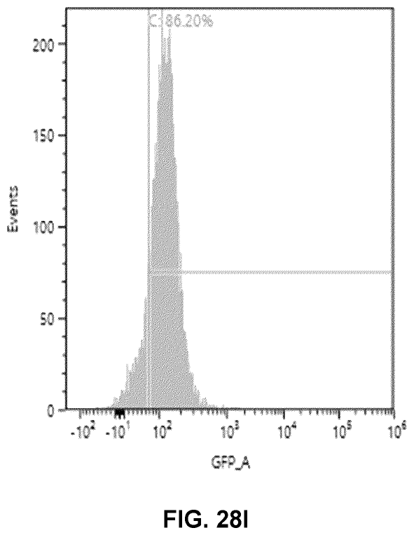

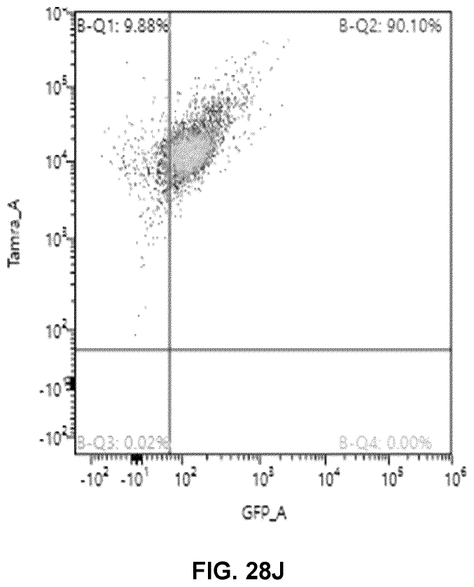

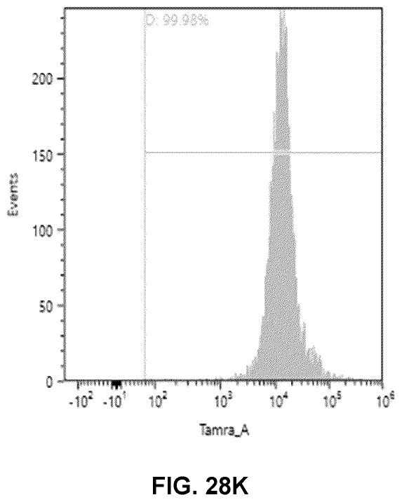

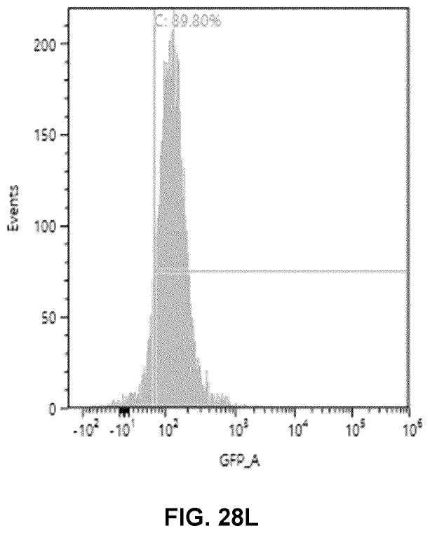

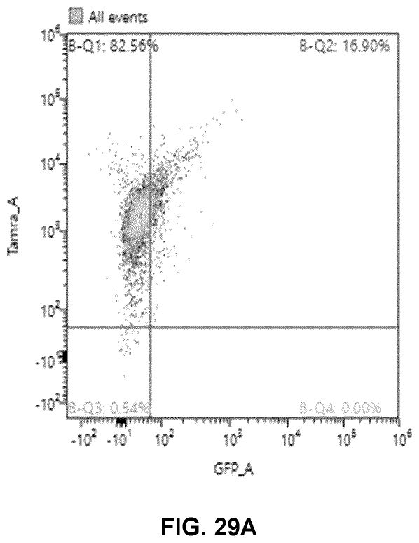

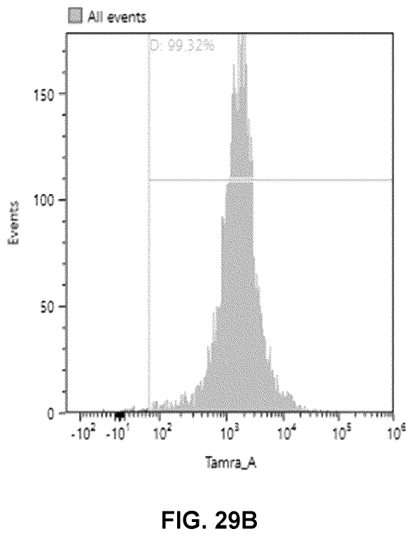

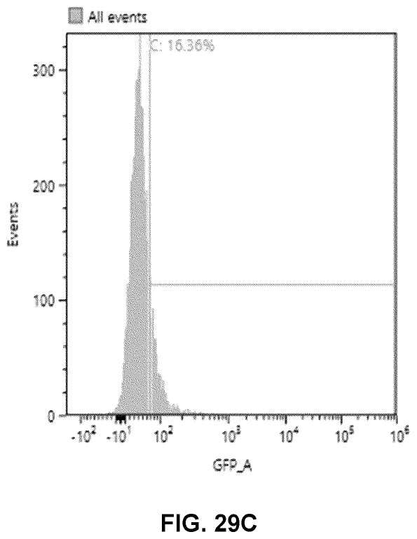

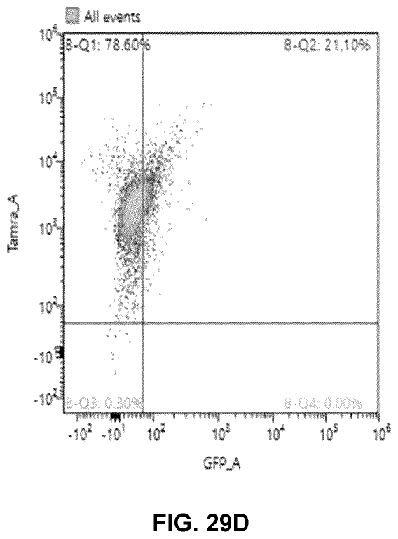

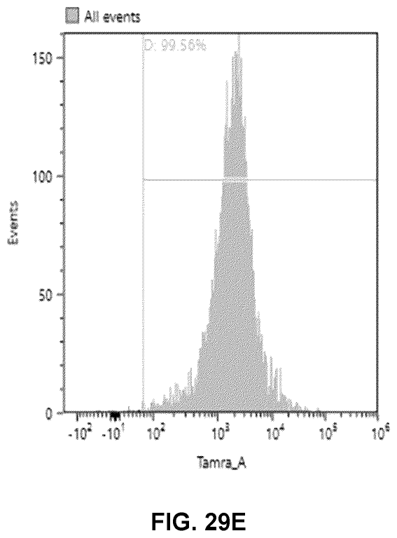

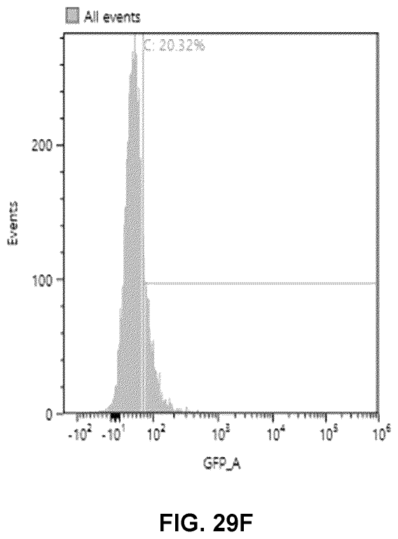

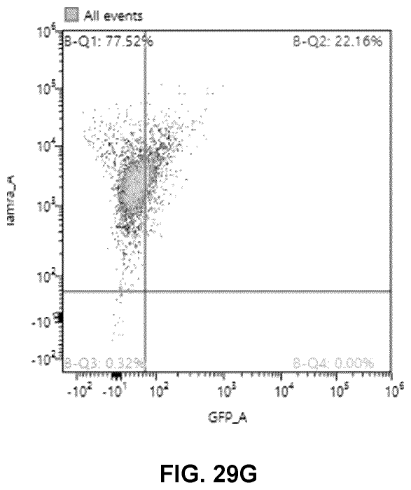

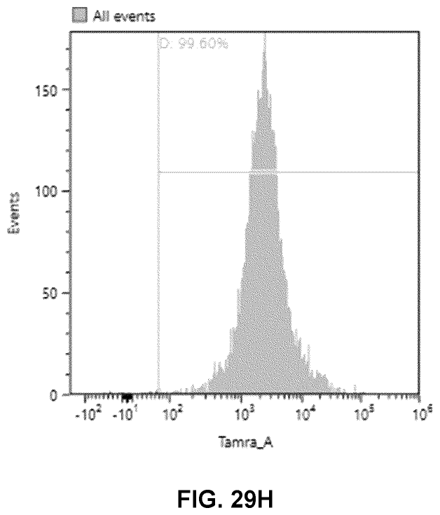

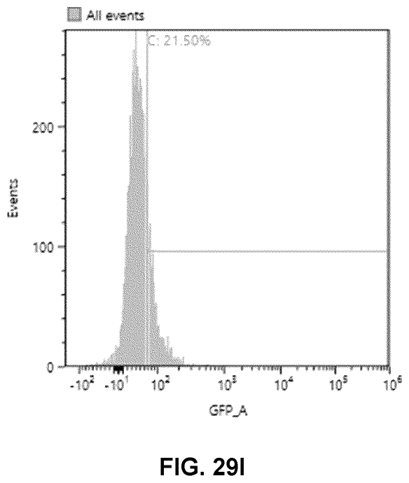

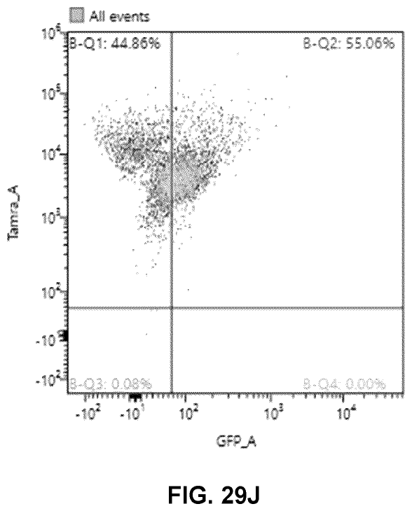

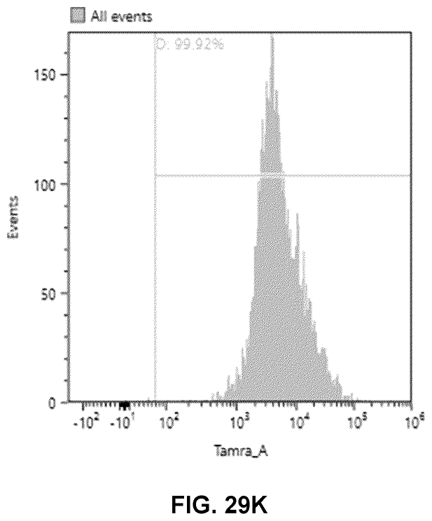

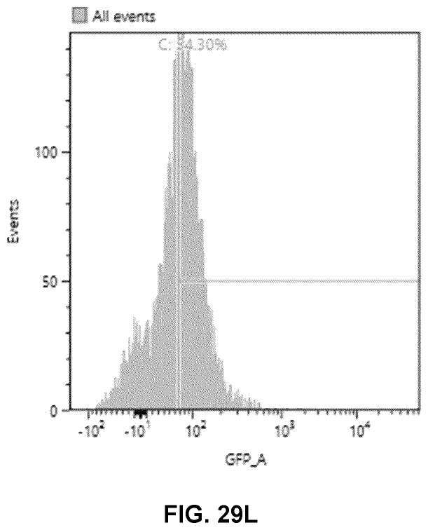

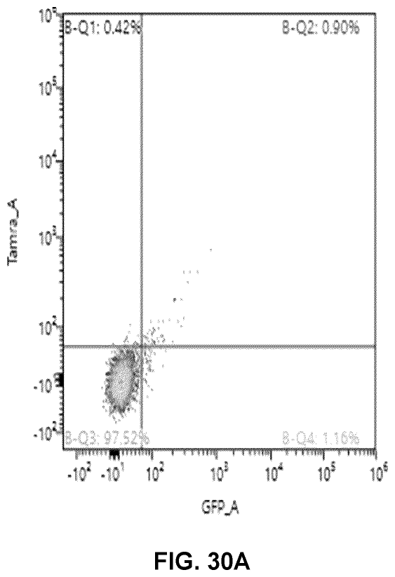

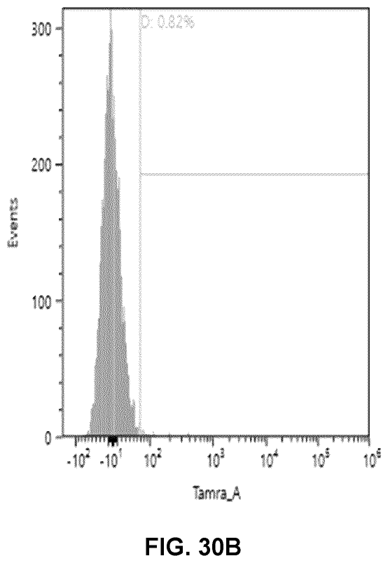

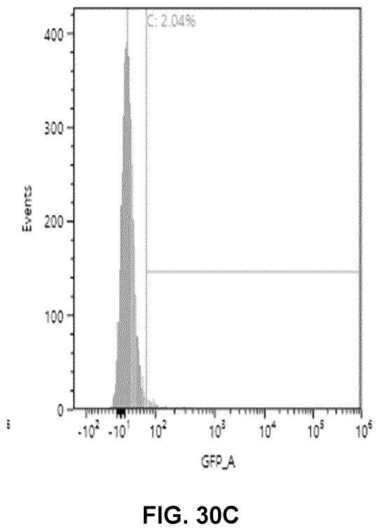

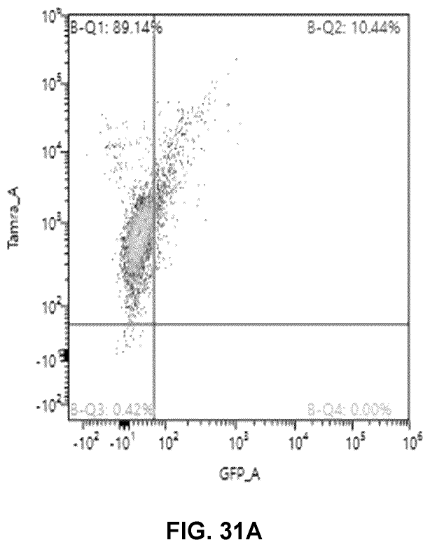

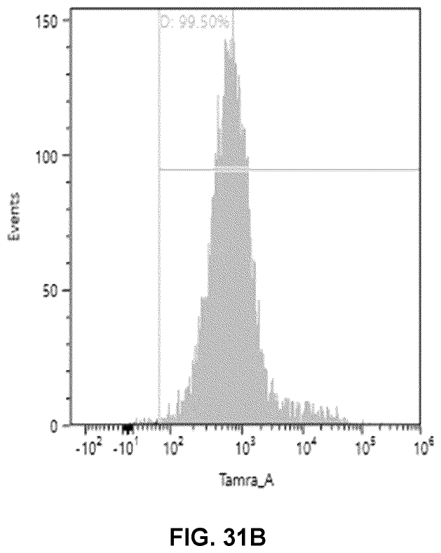

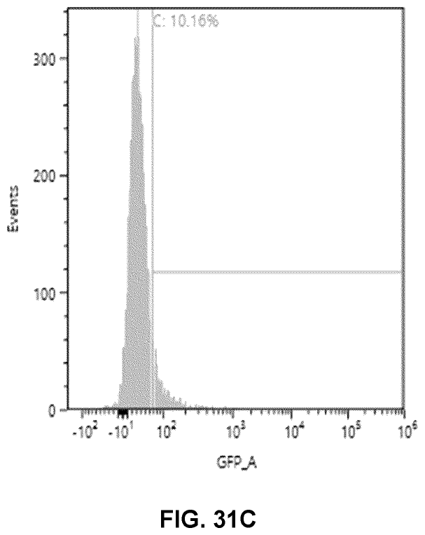

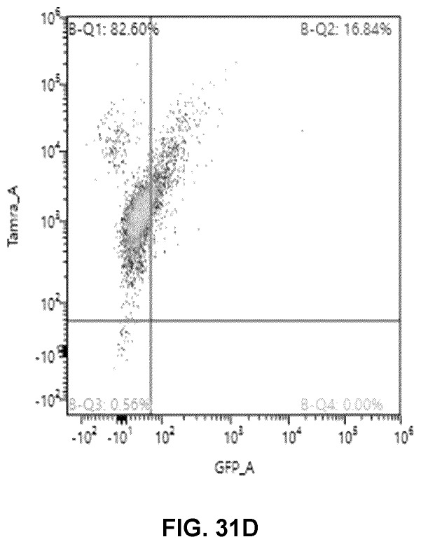

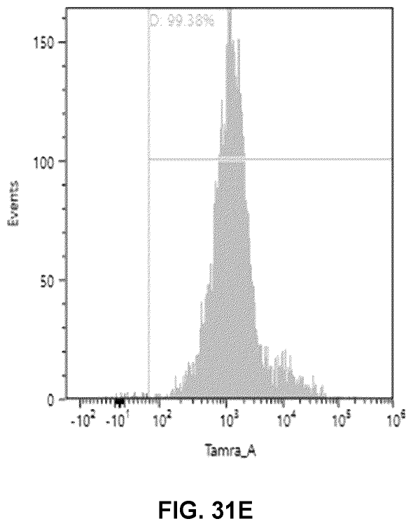

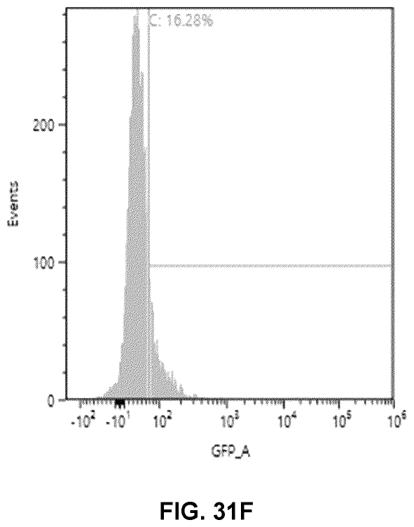

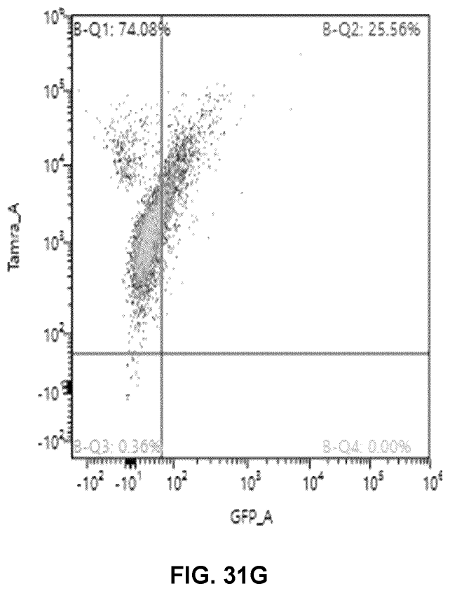

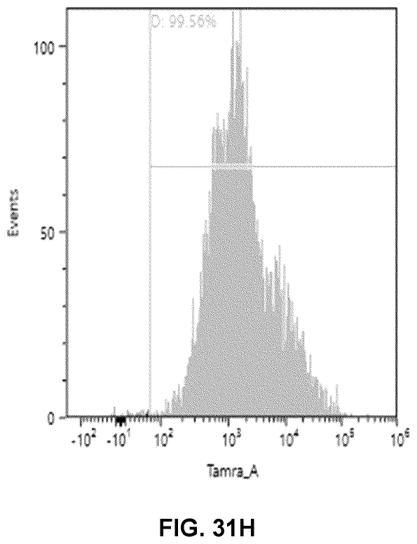

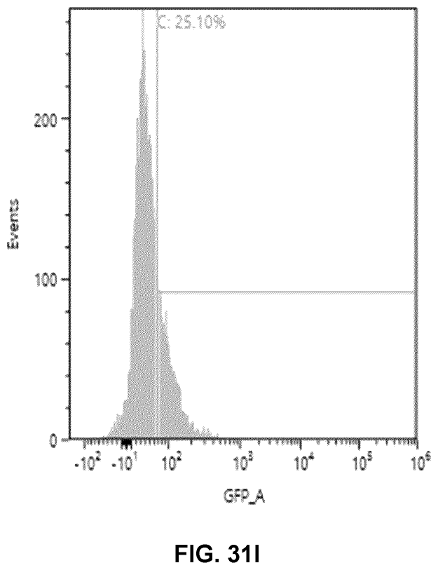

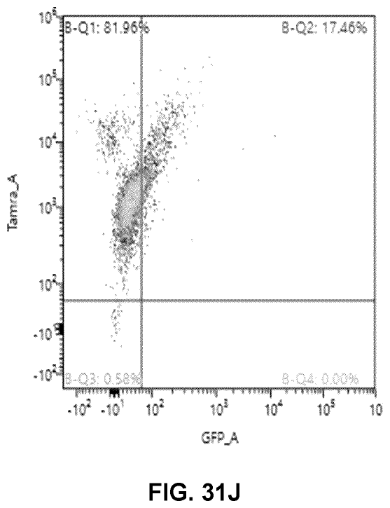

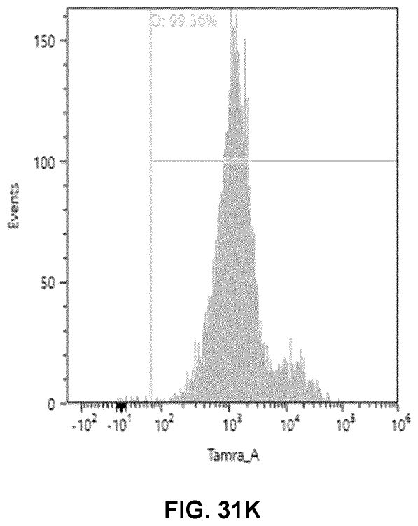

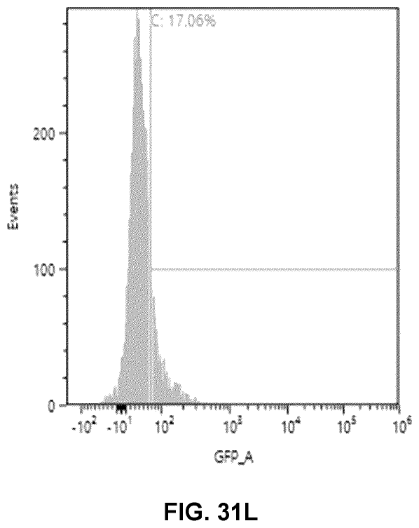

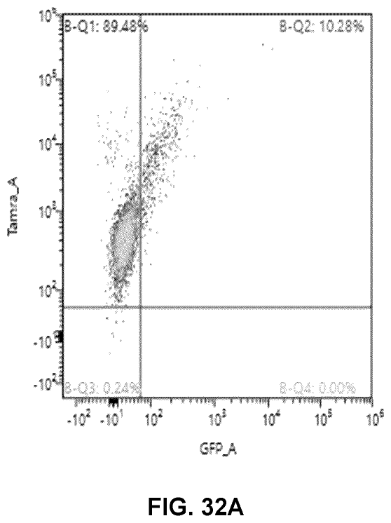

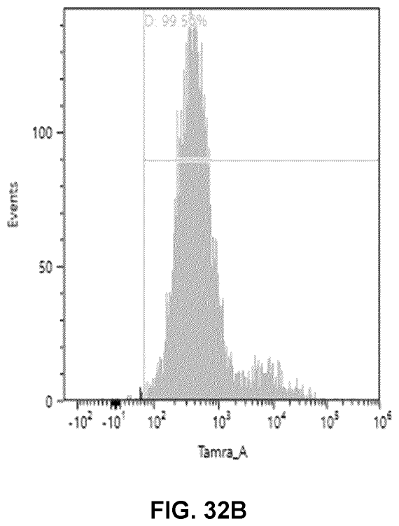

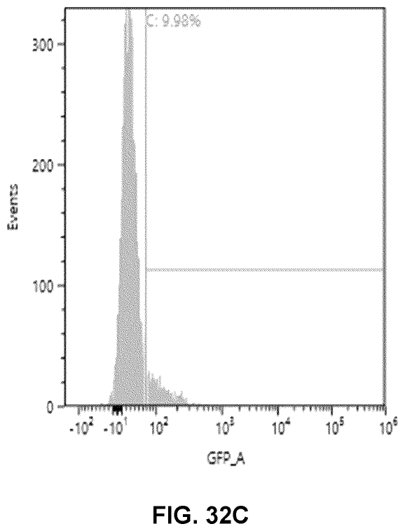

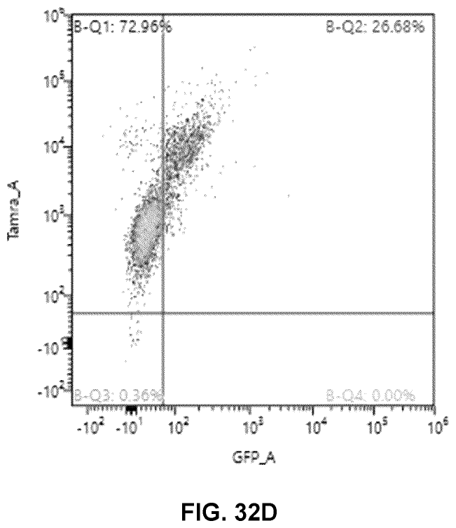

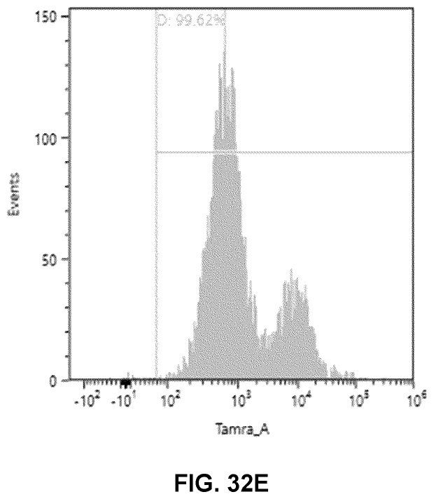

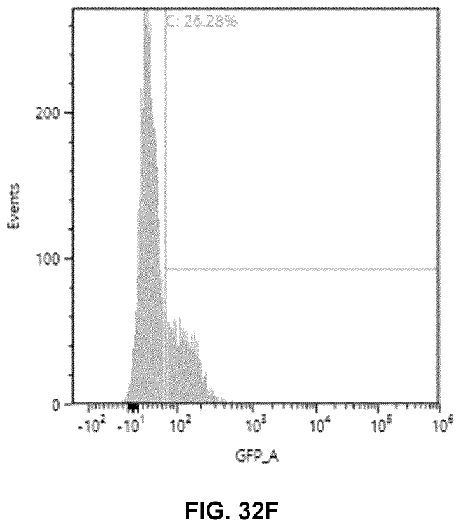

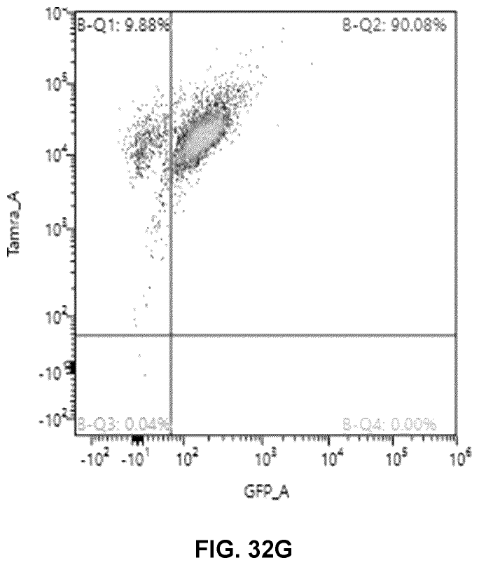

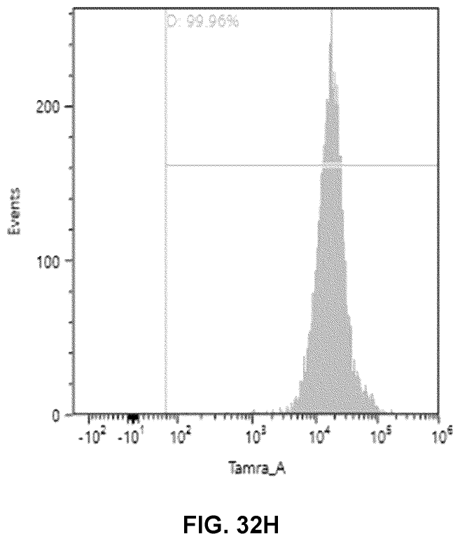

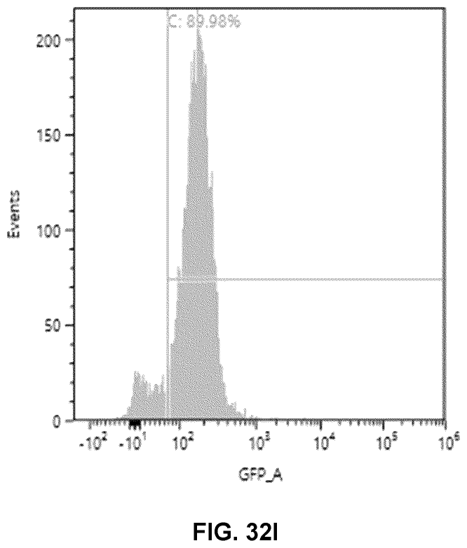

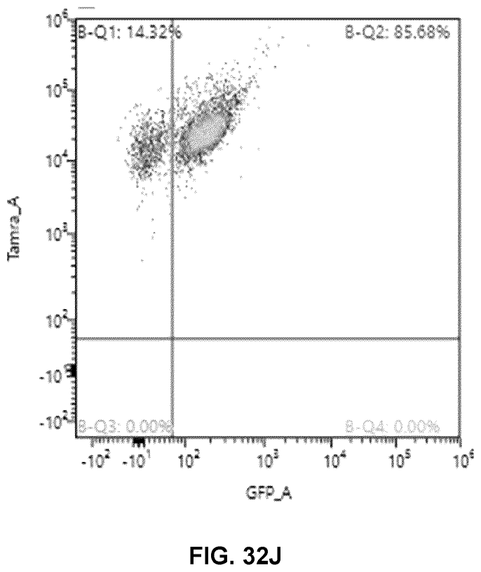

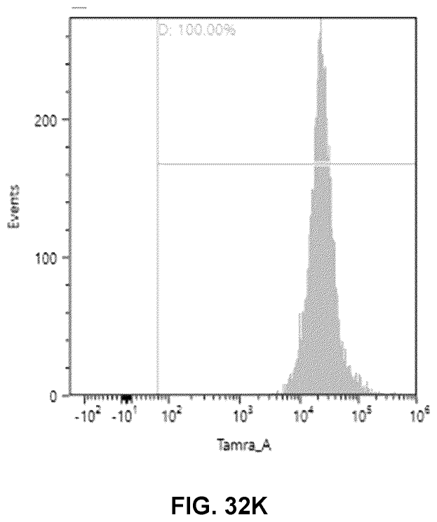

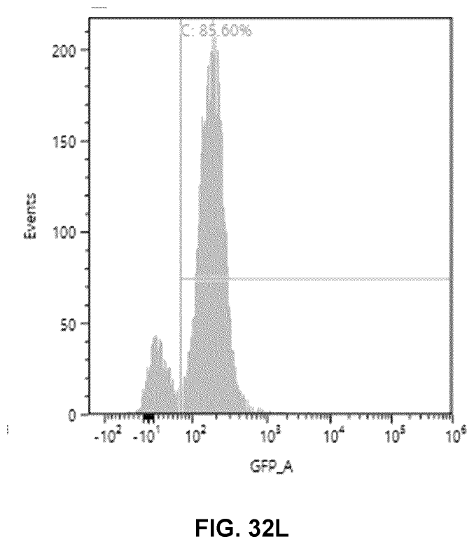

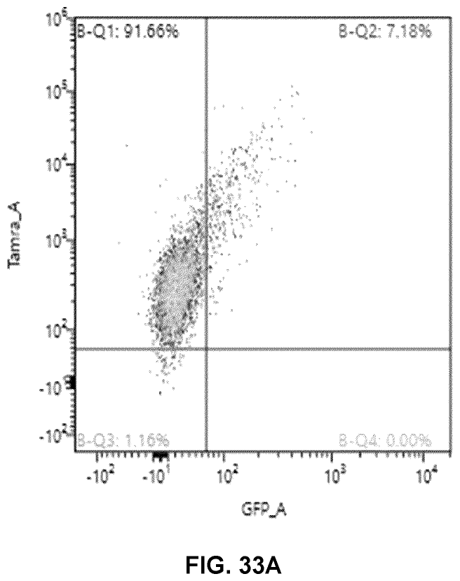

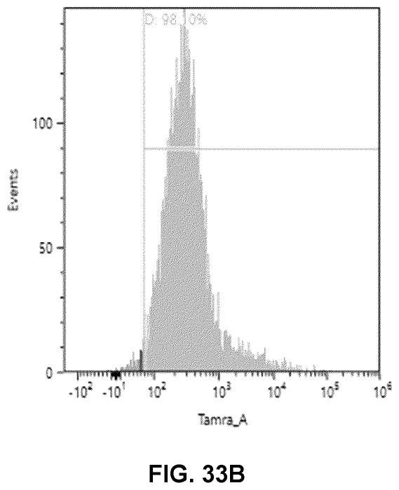

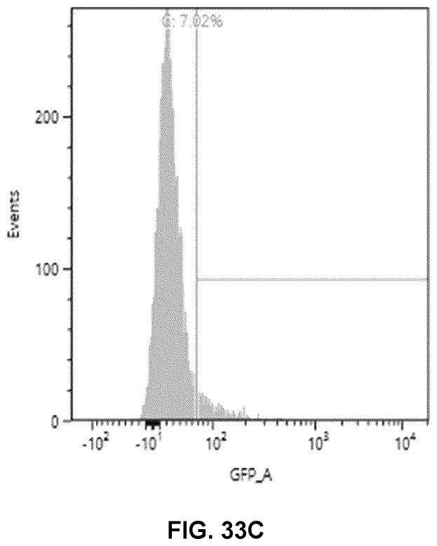

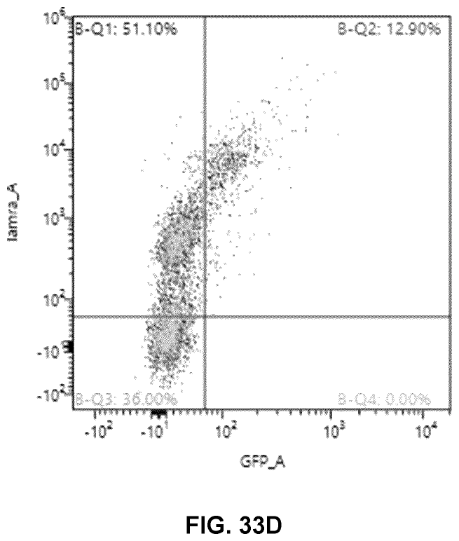

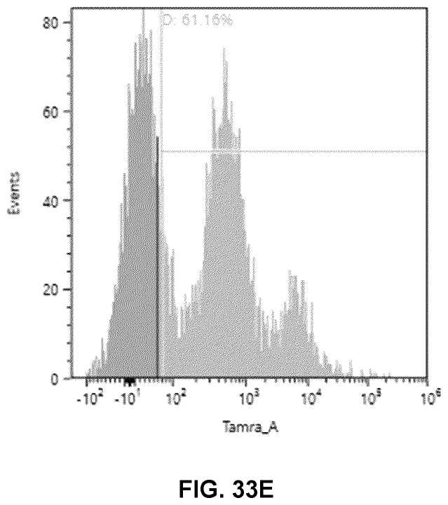

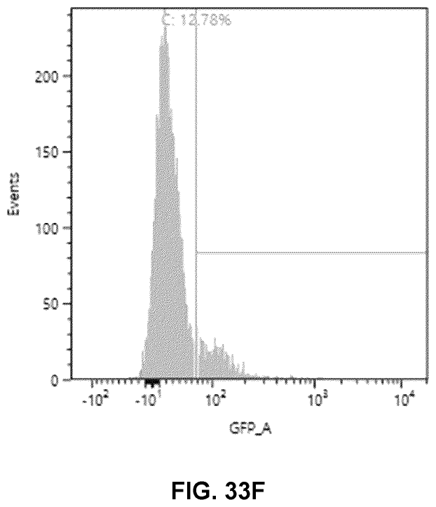

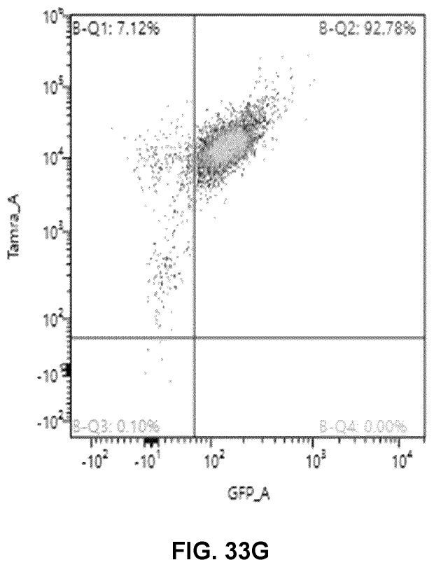

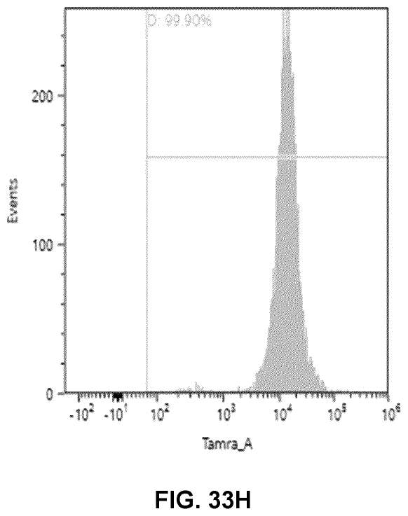

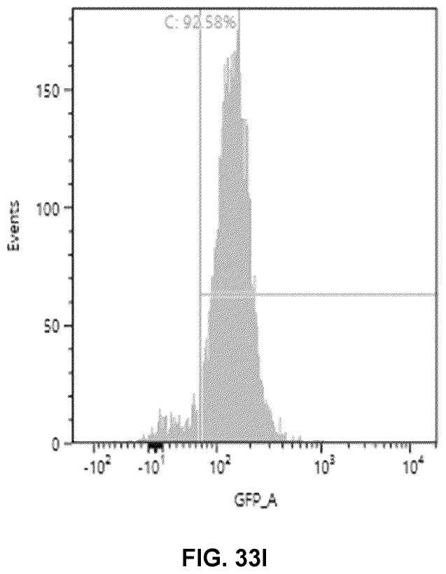

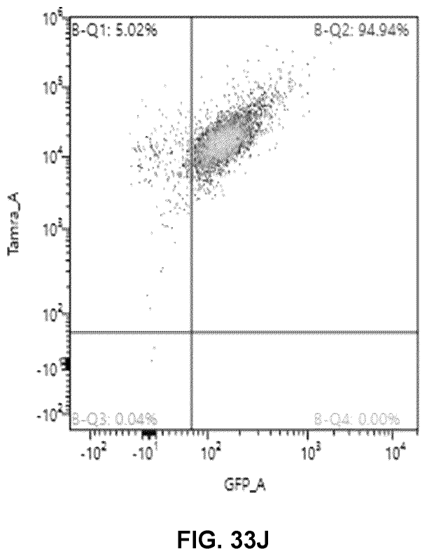

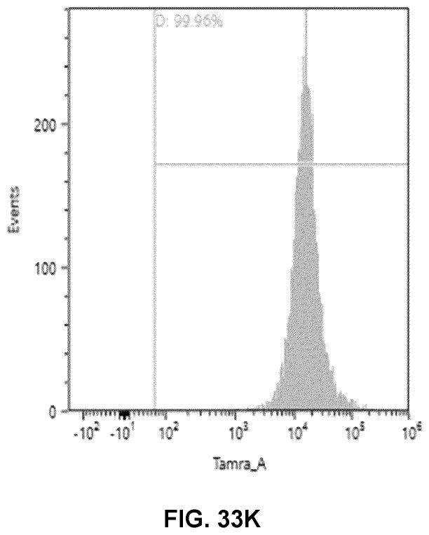

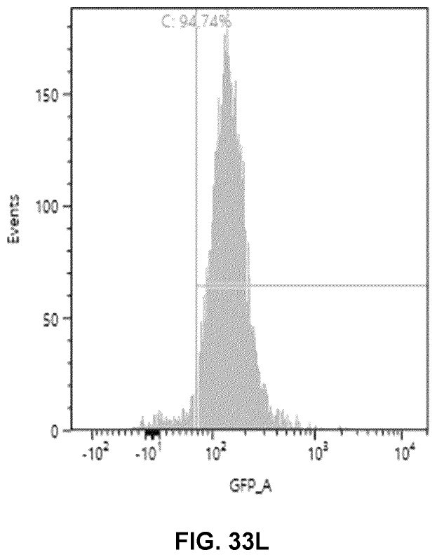

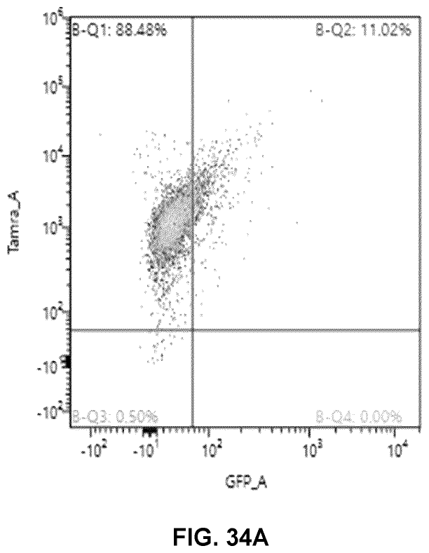

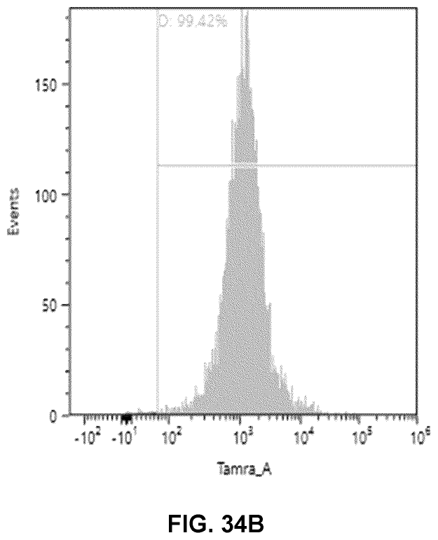

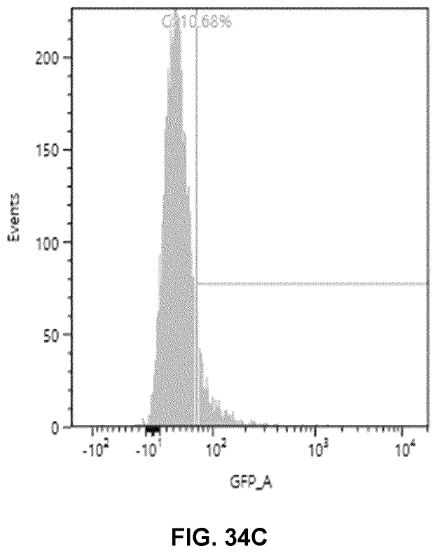

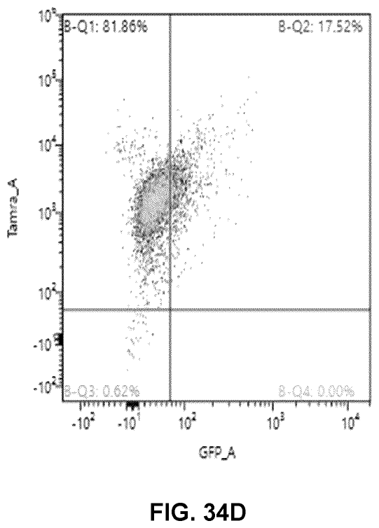

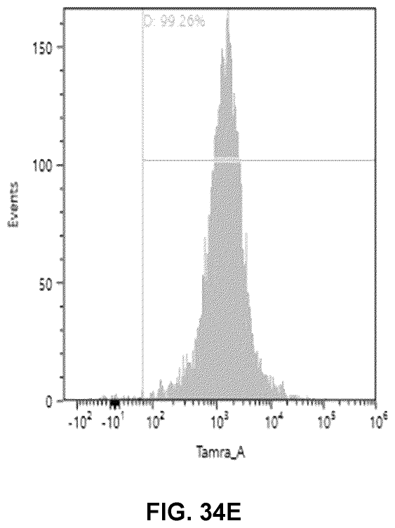

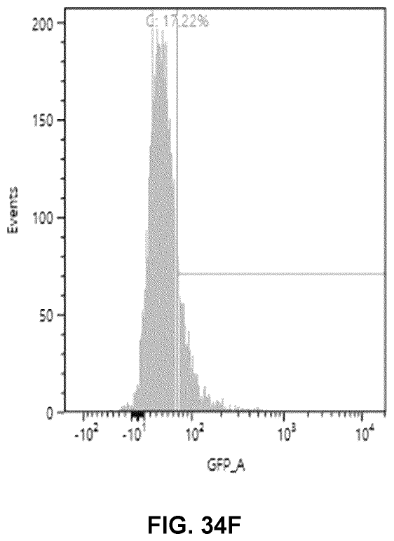

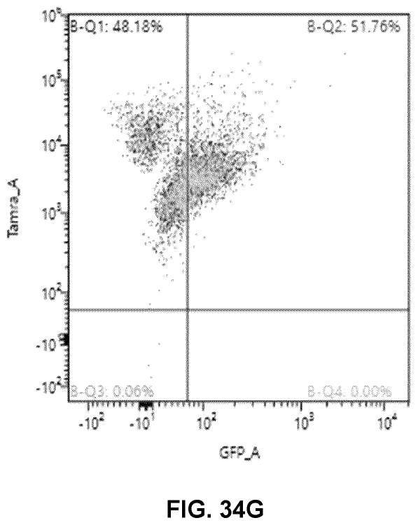

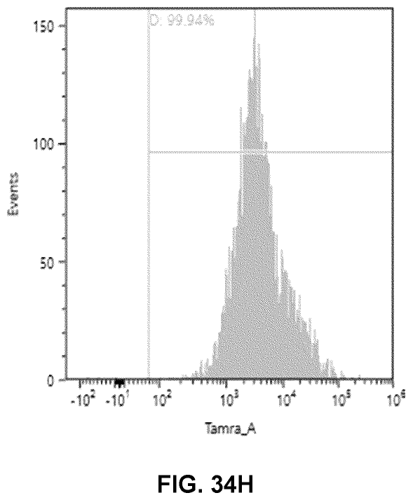

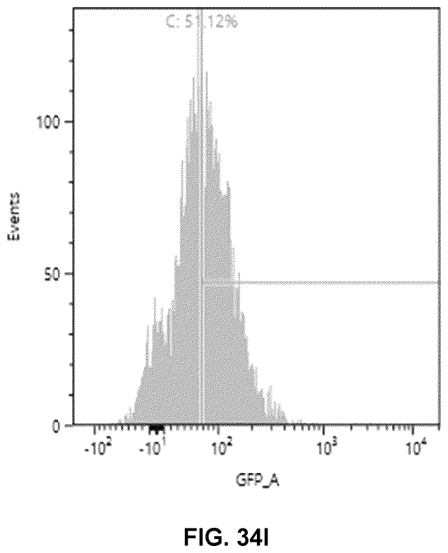

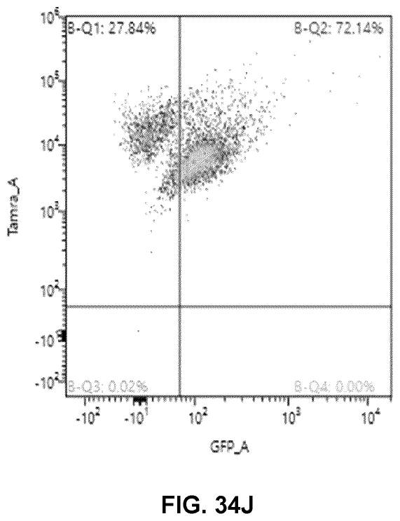

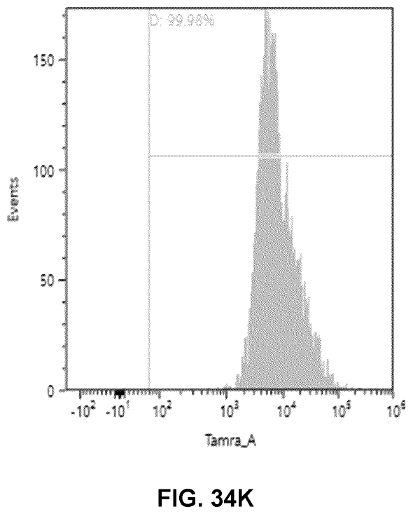

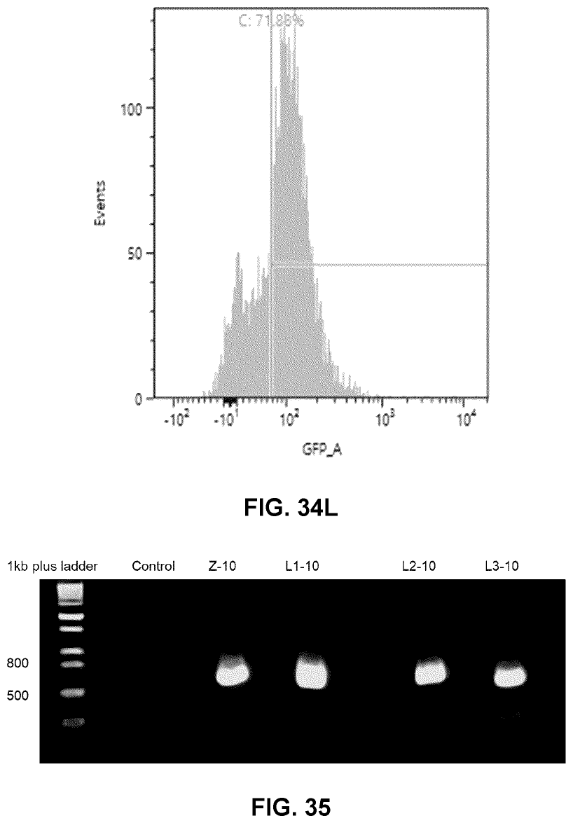

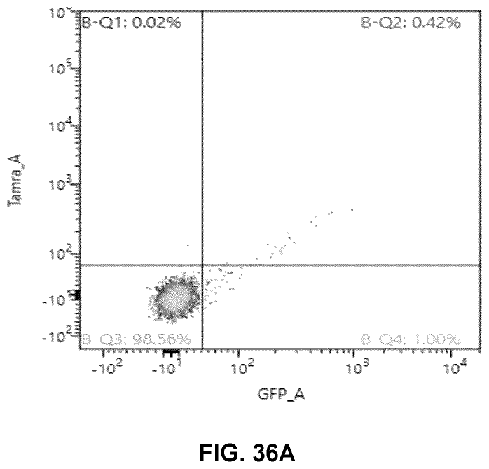

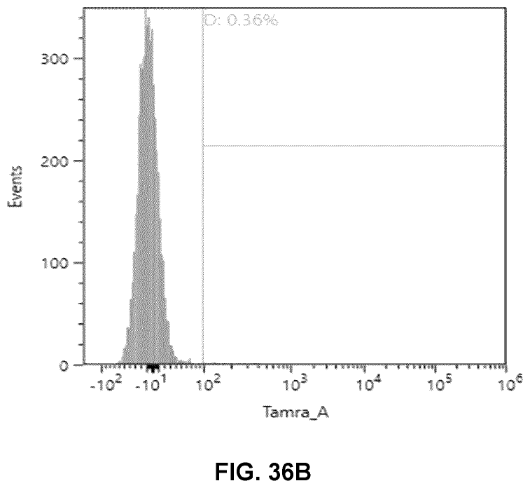

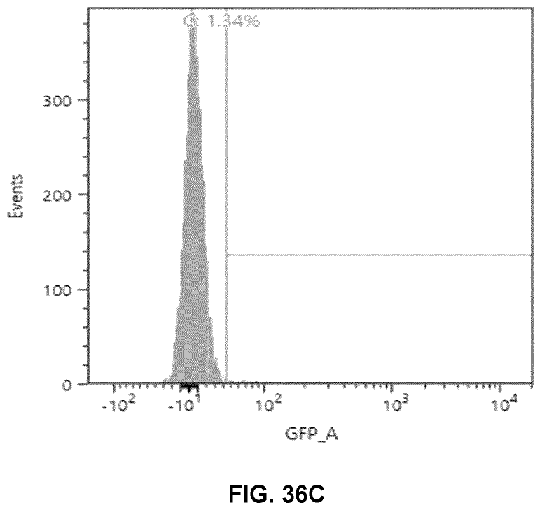

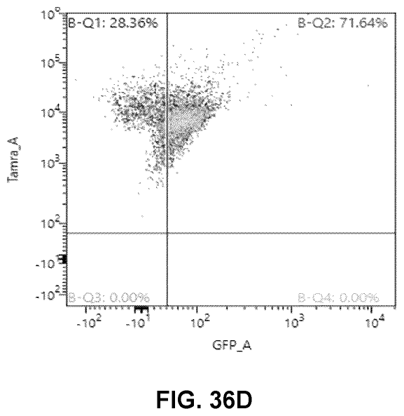

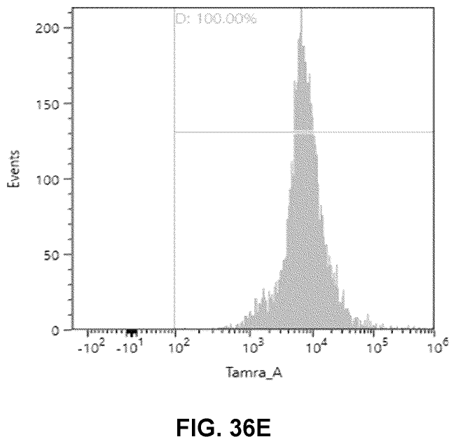

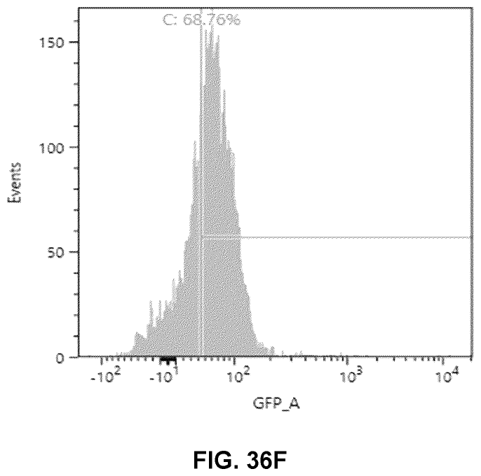

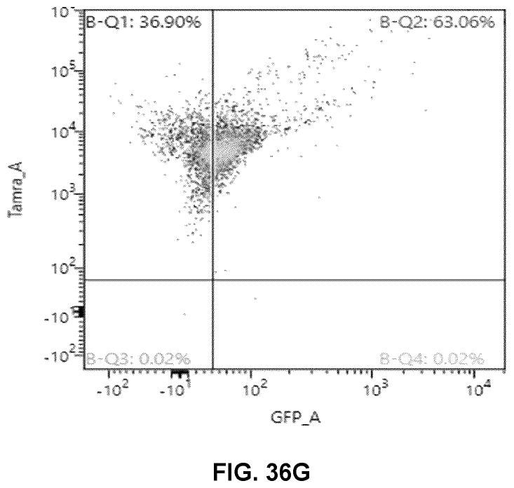

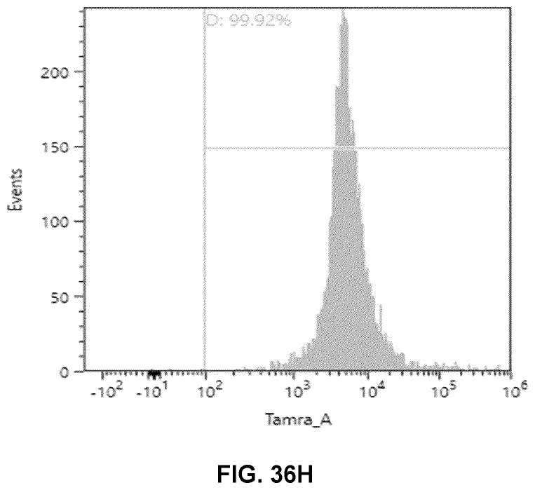

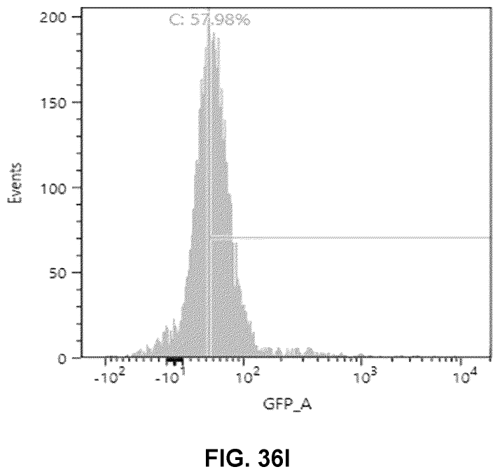

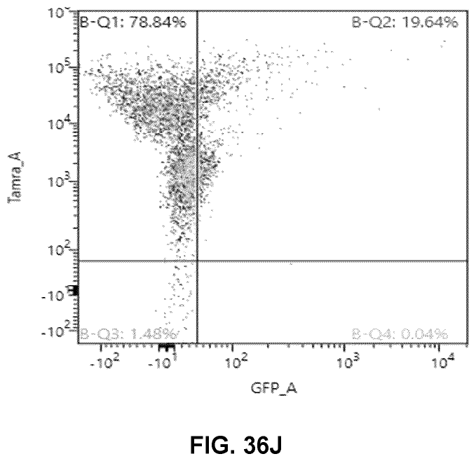

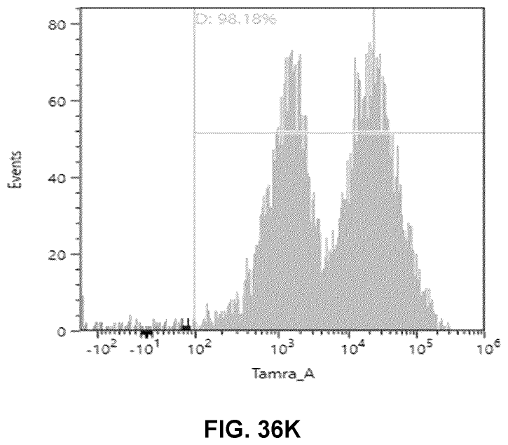

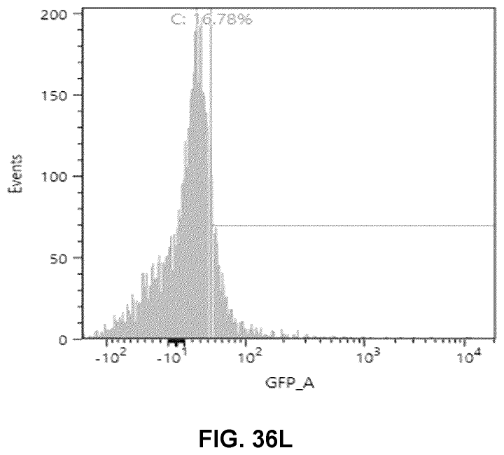

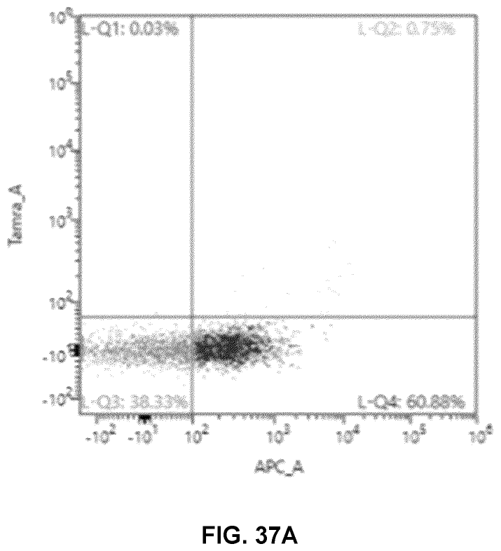

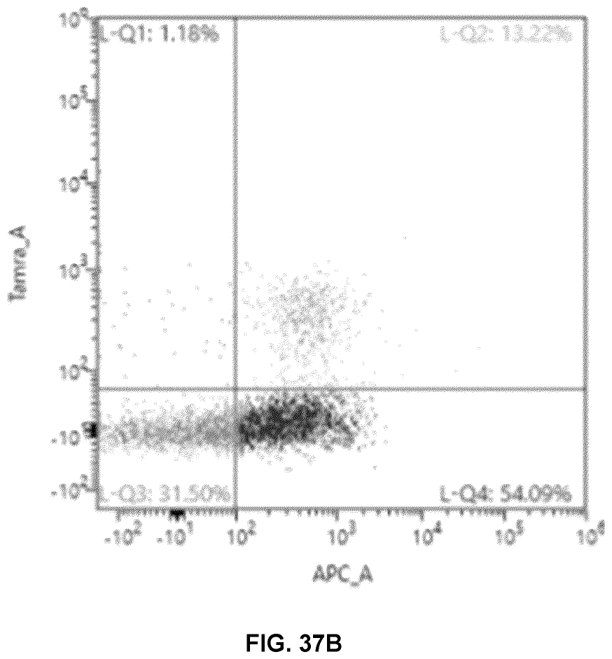

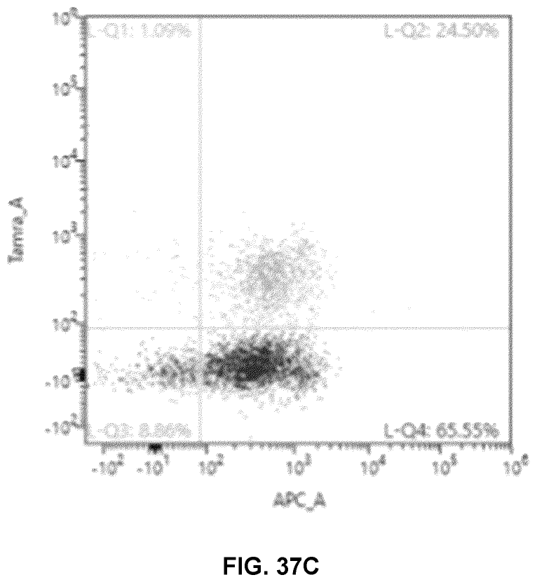

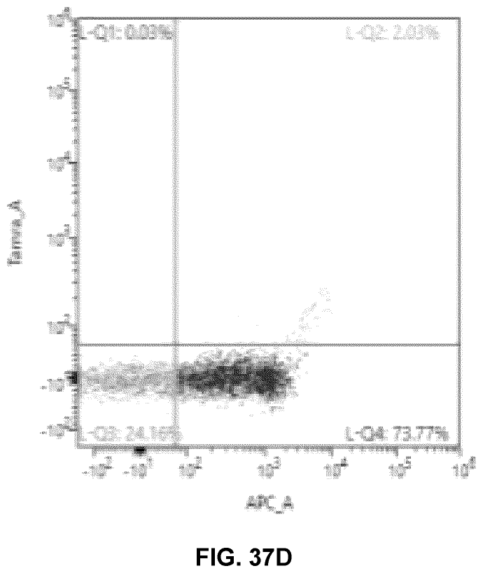

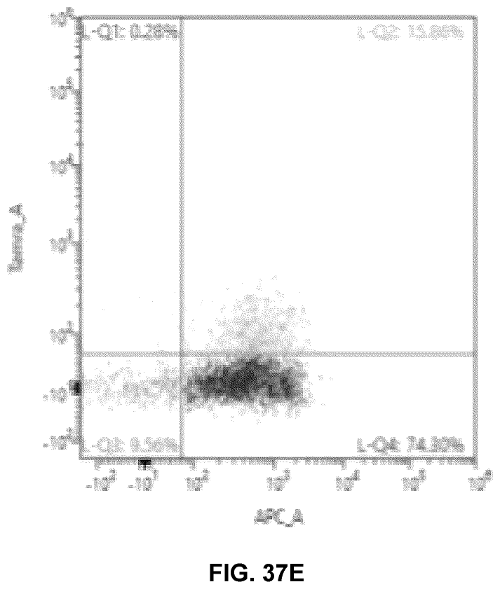

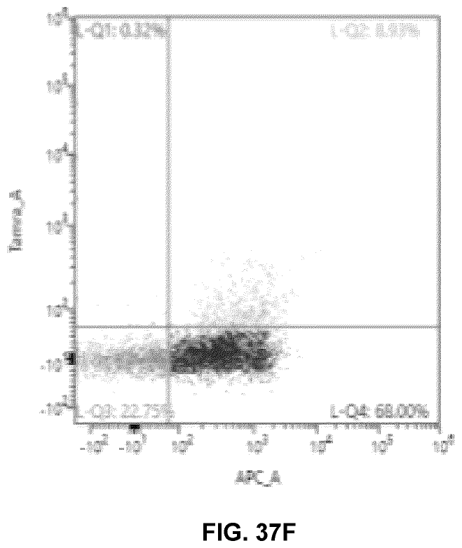

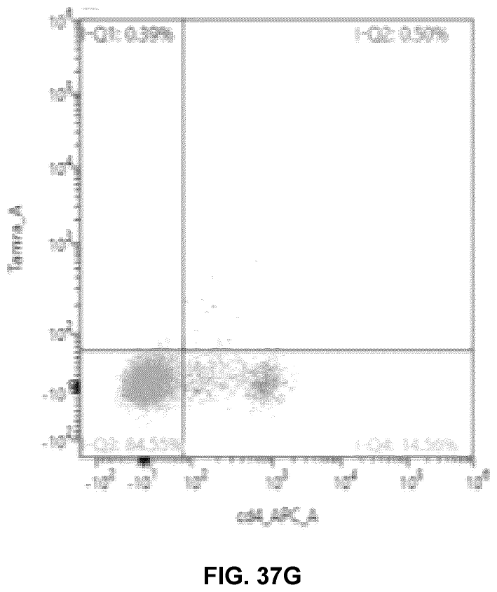

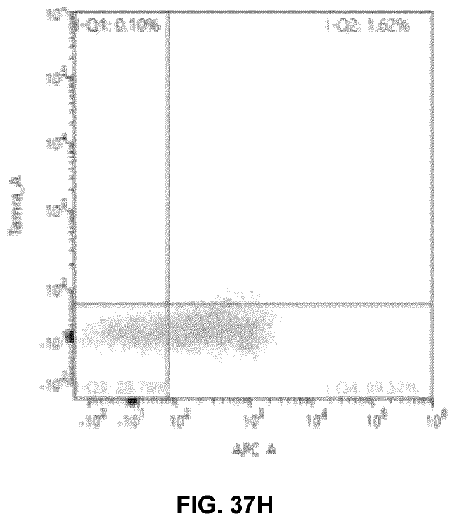

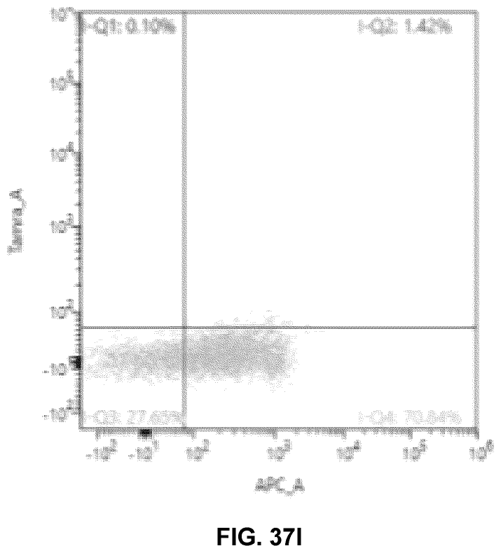

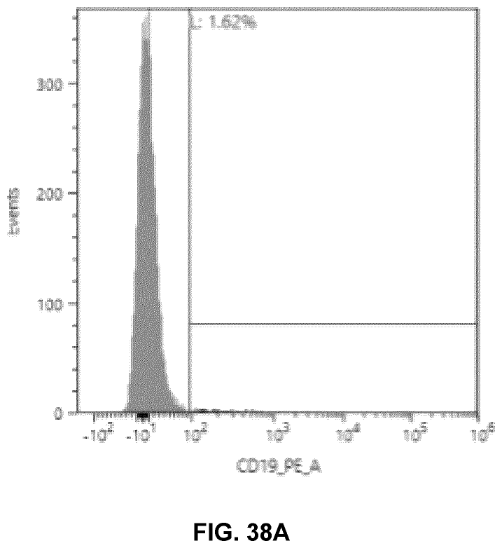

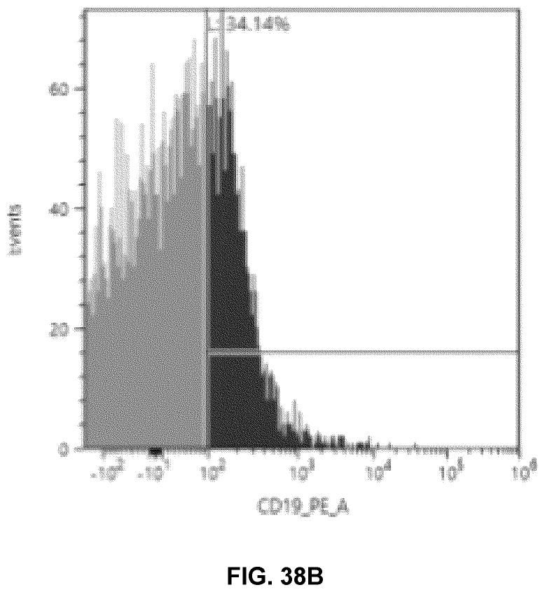

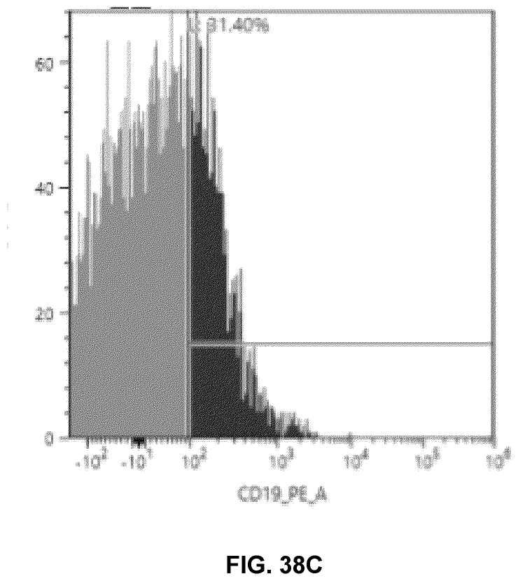

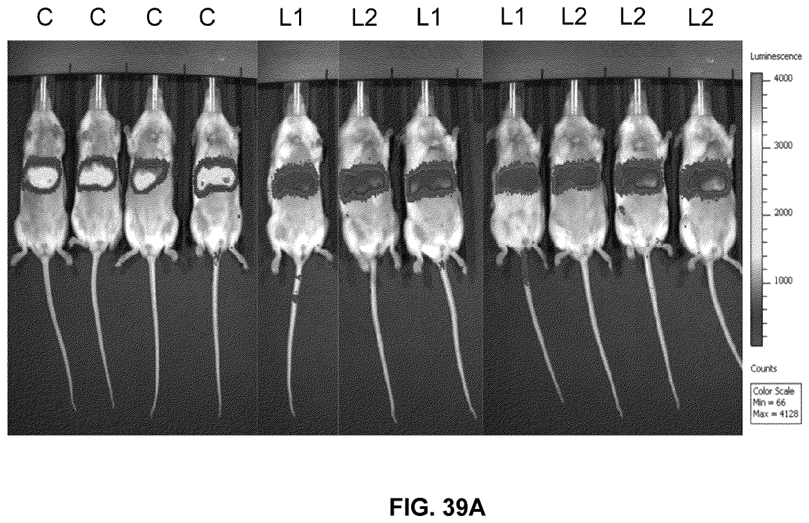

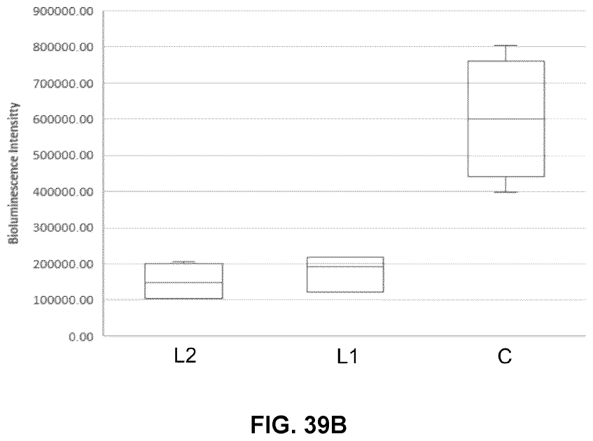

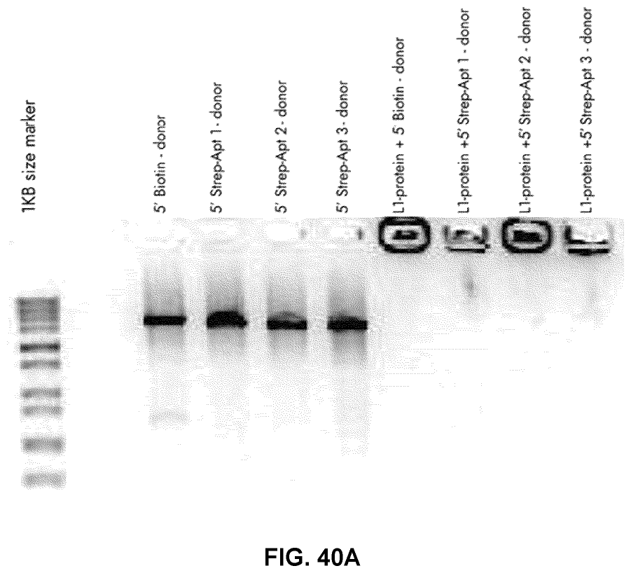

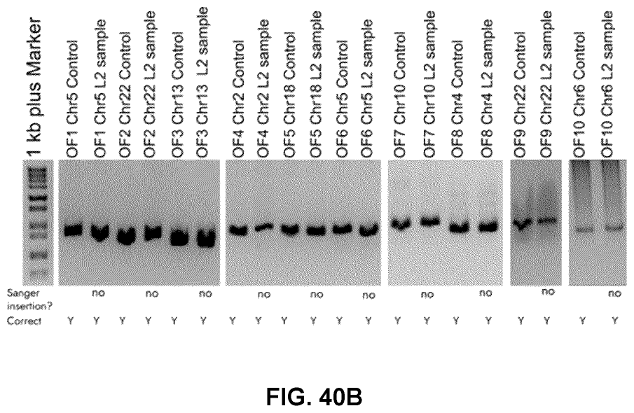

FIG. 26J is a flow cytometry graph showing the detection of TAMRA in function of GFP in the control condition (Zero in a concentration of 66 ng/μL in the Jurkat T-cell incubation of 48 hours).

FIG. 26K is a flow cytometry graph showing the events (×1000) in function of TAMRA detection in the control condition (Zero in a concentration of 66 ng/μL in the Jurkat T-cell incubation of 48 hours).

FIG. 26L is a flow cytometry graph showing the events (×1000) in function of GFP detection in the control condition (Zero in a concentration of 66 ng/μL in the Jurkat T-cell incubation of 48 hours).

FIG. 27A is a flow cytometry graph showing the detection of TAMRA in function of GFP in the control condition (L1 in a concentration of 0.33 ng/μL in the Jurkat T-cell incubation of 48 hours).

FIG. 27B is a flow cytometry graph showing the events (×1000) in function of TAMRA detection in the control condition (L1in a concentration of 0.33 ng/μL in the Jurkat T-cell incubation of 48 hours).

FIG. 27C is a flow cytometry graph showing the events (×1000) in function of GFP detection in the control condition (L1 in a concentration of 0.33 ng/μL in the Jurkat T-cell incubation of 48 hours).

FIG. 27D is a flow cytometry graph showing the detection of TAMRA in function of GFP in the control condition (L1 in a concentration of 16 ng/μL in the Jurkat T-cell incubation of 48 hours).

FIG. 27E is a flow cytometry graph showing the events (×1000) in function of TAMRA detection in the control condition (L1 in a concentration of 16 ng/μL in the Jurkat T-cell incubation of 48 hours).

FIG. 27F is a flow cytometry graph showing the events (×1000) in function of GFP detection in the control condition (L1 in a concentration of 16 ng/μL in the Jurkat T-cell incubation of 48 hours).

FIG. 27G is a flow cytometry graph showing the detection of TAMRA in function of GFP in the control condition (L1 in a concentration of 33.3 ng/μL in the Jurkat T-cell incubation of 48 hours).

FIG. 27H is a flow cytometry graph showing the events (×1000) in function of TAMRA detection in the control condition (L1 in a concentration of 33.3 ng/μL in the Jurkat T-cell incubation of 48 hours).

FIG. 271 is a flow cytometry graph showing the events (×1000) in function of GFP detection in the control condition (L1 in a concentration of 33.3 ng/μL in the Jurkat T-cell incubation of 48 hours).

FIG. 27J is a flow cytometry graph showing the detection of TAMRA in function of GFP in the control condition (L1 in a concentration of 66 ng/μL in the Jurkat T-cell incubation of 48 hours).

FIG. 27K is a flow cytometry graph showing the events (×1000) in function of TAMRA detection in the control condition (L1 in a concentration of 66 ng/μL in the Jurkat T-cell incubation of 48 hours).

FIG. 27L is a flow cytometry graph showing the events (×1000) in function of GFP detection in the control condition (L1 in a concentration of 66 ng/μL in the Jurkat T-cell incubation of 48 hours).

FIG. 28A is a flow cytometry graph showing the detection of TAMRA in function of GFP in the control condition (L2 in a concentration of 0.33 ng/μL in the Jurkat T-cell incubation of 48 hours).

FIG. 28B is a flow cytometry graph showing the events (×1000) in function of TAMRA detection in the control condition (L2 in a concentration of 0.33 ng/μL in the Jurkat T-cell incubation of 48 hours).

FIG. 28C is a flow cytometry graph showing the events (×1000) in function of GFP detection in the control condition (L2 in a concentration of 0.33 ng/μL in the Jurkat T-cell incubation of 48 hours).

FIG. 28D is a flow cytometry graph showing the detection of TAMRA in function of GFP in the control condition (L2 in a concentration of 16 ng/μL in the Jurkat T-cell incubation of 48 hours).

FIG. 28E is a flow cytometry graph showing the events (×1000) in function of TAMRA detection in the control condition (L2 in a concentration of 16 ng/μL in the Jurkat T-cell incubation of 48 hours).

FIG. 28F is a flow cytometry graph showing the events (×1000) in function of GFP detection in the control condition (L2 in a concentration of 16 ng/μL in the Jurkat T-cell incubation of 48 hours).

FIG. 28G is a flow cytometry graph showing the detection of TAMRA in function of GFP in the control condition (L2 in a concentration of 33.3 ng/μL in the Jurkat T-cell incubation of 48 hours).

FIG. 28H is a flow cytometry graph showing the events (×1000) in function of TAMRA detection in the control condition (L2 in a concentration of 33.3 ng/μL in the Jurkat T-cell incubation of 48 hours).

FIG. 28I is a flow cytometry graph showing the events (×1000) in function of GFP detection in the control condition (L2 in a concentration of 33.3 ng/μL in the Jurkat T-cell incubation of 48 hours).

FIG. 28J is a flow cytometry graph showing the detection of TAMRA in function of GFP in the control condition (L2 in a concentration of 66 ng/μL in the Jurkat T-cell incubation of 48 hours).

FIG. 28K is a flow cytometry graph showing the events (×1000) in function of TAMRA detection in the control condition (L2 in a concentration of 66 ng/μL in the Jurkat T-cell incubation of 48 hours).

FIG. 28L is a flow cytometry graph showing the events (×1000) in function of GFP detection in the control condition (L2 in a concentration of 66 ng/μL in the Jurkat T-cell incubation of 48 hours).

FIG. 29A is a flow cytometry graph showing the detection of TAMRA in function of GFP in the control condition (L3 in a concentration of 0.33 ng/μL in the Jurkat T-cell incubation of 48 hours).

FIG. 29B is a flow cytometry graph showing the events (×1000) in function of TAMRA detection in the control condition (L3 in a concentration of 0.33 ng/μL in the Jurkat T-cell incubation of 48 hours).

FIG. 29C is a flow cytometry graph showing the events (×1000) in function of GFP detection in the control condition (L3 in a concentration of 0.33 ng/μL in the Jurkat T-cell incubation of 48 hours).

FIG. 29D is a flow cytometry graph showing the detection of TAMRA in function of GFP in the control condition (L3 in a concentration of 16 ng/μL in the Jurkat T-cell incubation of 48 hours).

FIG. 29E is a flow cytometry graph showing the events (×1000) in function of TAMRA detection in the control condition (L3 in a concentration of 16 ng/μL in the Jurkat T-cell incubation of 48 hours).

FIG. 29F is a flow cytometry graph showing the events (×1000) in function of GFP detection in the control condition (L3 in a concentration of 16 ng/μL in the Jurkat T-cell incubation of 48 hours).

FIG. 29G is a flow cytometry graph showing the detection of TAMRA in function of GFP in the control condition (L3 in a concentration of 33.3 ng/μL in the Jurkat T-cell incubation of 48 hours).

FIG. 29H is a flow cytometry graph showing the events (×1000) in function of TAMRA detection in the control condition (L3 in a concentration of 33.3 ng/μL in the Jurkat T-cell incubation of 48 hours).

FIG. 291 is a flow cytometry graph showing the events (×1000) in function of GFP detection in the control condition (L3 in a concentration of 33.3 ng/μL in the Jurkat T-cell incubation of 48 hours).

FIG. 29J is a flow cytometry graph showing the detection of TAMRA in function of GFP in the control condition (L3 in a concentration of 66 ng/μL in the Jurkat T-cell incubation of 48 hours).

FIG. 29K is a flow cytometry graph showing the events (×1000) in function of TAMRA detection in the control condition (L3 in a concentration of 66 ng/μL in the Jurkat T-cell incubation of 48 hours).

FIG. 29L is a flow cytometry graph showing the events (×1000) in function of GFP detection in the control condition (L3 in a concentration of 66 ng/μL in the Jurkat T-cell incubation of 48 hours).

FIG. 30A is a flow cytometry graph showing the detection of TAMRA in function of GFP in the control condition (no nuclease provided in the Jurkat T-cell incubation of 72 hours).

FIG. 30B is a flow cytometry graph showing the events (×1000) in function of TAMRA detection in the control condition (no nuclease provided in the Jurkat T-cell incubation of 72 hours).

FIG. 30C is a flow cytometry graph showing the events (×1000) in function of GFP detection in the control condition (no nuclease provided in the Jurkat T-cell incubation of 72 hours).

FIG. 31A is a flow cytometry graph showing the detection of TAMRA in function of GFP in the control condition (Zero in a concentration of 0.33 ng/μL in the Jurkat T-cell incubation of 72 hours).

FIG. 31B is a flow cytometry graph showing the events (×1000) in function of TAMRA detection in the control condition (Zero in a concentration of 0.33 ng/μL in the Jurkat T-cell incubation of 72 hours).

FIG. 31C is a flow cytometry graph showing the events (×1000) in function of GFP detection in the control condition (Zero in a concentration of 0.33 ng/μL in the Jurkat T-cell incubation of 72 hours).

FIG. 31D is a flow cytometry graph showing the detection of TAMRA in function of GFP in the control condition (Zero in a concentration of 16 ng/μL in the Jurkat T-cell incubation of 72 hours).

FIG. 31E is a flow cytometry graph showing the events (×1000) in function of TAMRA detection in the control condition (Zero in a concentration of 16 ng/μL in the Jurkat T-cell incubation of 72 hours).

FIG. 31F is a flow cytometry graph showing the events (×1000) in function of GFP detection in the control condition (Zero in a concentration of 16 ng/μL in the Jurkat T-cell incubation of 72 hours).

FIG. 31G is a flow cytometry graph showing the detection of TAMRA in function of GFP in the control condition (Zero in a concentration of 33.3 ng/μL in the Jurkat T-cell incubation of 72 hours).

FIG. 31H is a flow cytometry graph showing the events (×1000) in function of TAMRA detection in the control condition (Zero in a concentration of 33.3 ng/μL in the Jurkat T-cell incubation of 72 hours).

FIG. 311 is a flow cytometry graph showing the events (×1000) in function of GFP detection in the control condition (Zero in a concentration of 33.3 ng/μL in the Jurkat T-cell incubation of 72 hours).

FIG. 31J is a flow cytometry graph showing the detection of TAMRA in function of GFP in the control condition (Zero in a concentration of 66 ng/μL in the Jurkat T-cell incubation of 72 hours).

FIG. 31K is a flow cytometry graph showing the events (×1000) in function of TAMRA detection in the control condition (Zero in a concentration of 66 ng/μL in the Jurkat T-cell incubation of 72 hours).

FIG. 31L is a flow cytometry graph showing the events (×1000) in function of GFP detection in the control condition (Zero in a concentration of 66 ng/μL in the Jurkat T-cell incubation of 72 hours).

FIG. 32A is a flow cytometry graph showing the detection of TAMRA in function of GFP in the control condition (L1 in a concentration of 0.33 ng/μL in the Jurkat T-cell incubation of 72 hours).

FIG. 32B is a flow cytometry graph showing the events (×1000) in function of TAMRA detection in the control condition (L1in a concentration of 0.33 ng/μL in the Jurkat T-cell incubation of 72 hours).

FIG. 32C is a flow cytometry graph showing the events (×1000) in function of GFP detection in the control condition (L1 in a concentration of 0.33 ng/μL in the Jurkat T-cell incubation of 72 hours).

FIG. 32D is a flow cytometry graph showing the detection of TAMRA in function of GFP in the control condition (L1 in a concentration of 16 ng/μL in the Jurkat T-cell incubation of 72 hours).

FIG. 32E is a flow cytometry graph showing the events (×1000) in function of TAMRA detection in the control condition (L1 in a concentration of 16 ng/μL in the Jurkat T-cell incubation of 72 hours).

FIG. 32F is a flow cytometry graph showing the events (×1000) in function of GFP detection in the control condition (L1 in a concentration of 16 ng/μL in the Jurkat T-cell incubation of 72 hours).

FIG. 32G is a flow cytometry graph showing the detection of TAMRA in function of GFP in the control condition (L1 in a concentration of 33.3 ng/μL in the Jurkat T-cell incubation of 72 hours).

FIG. 32H is a flow cytometry graph showing the events (×1000) in function of TAMRA detection in the control condition (L1 in a concentration of 33.3 ng/μL in the Jurkat T-cell incubation of 72 hours).

FIG. 321 is a flow cytometry graph showing the events (×1000) in function of GFP detection in the control condition (L1 in a concentration of 33.3 ng/μL in the Jurkat T-cell incubation of 72 hours).

FIG. 32J is a flow cytometry graph showing the detection of TAMRA in function of GFP in the control condition (L1 in a concentration of 66 ng/μL in the Jurkat T-cell incubation of 72 hours).

FIG. 32K is a flow cytometry graph showing the events (×1000) in function of TAMRA detection in the control condition (L1 in a concentration of 66 ng/μL in the Jurkat T-cell incubation of 72 hours).

FIG. 32L is a flow cytometry graph showing the events (×1000) in function of GFP detection in the control condition (L1 in a concentration of 66 ng/μL in the Jurkat T-cell incubation of 72 hours).

FIG. 33A is a flow cytometry graph showing the detection of TAMRA in function of GFP in the control condition (L2 in a concentration of 0.33 ng/μL in the Jurkat T-cell incubation of 72 hours).

FIG. 33B is a flow cytometry graph showing the events (×1000) in function of TAMRA detection in the control condition (L2 in a concentration of 0.33 ng/μL in the Jurkat T-cell incubation of 72 hours).

FIG. 33C is a flow cytometry graph showing the events (×1000) in function of GFP detection in the control condition (L2 in a concentration of 0.33 ng/μL in the Jurkat T-cell incubation of 72 hours).

FIG. 33D is a flow cytometry graph showing the detection of TAMRA in function of GFP in the control condition (L2 in a concentration of 16 ng/μL in the Jurkat T-cell incubation of 72 hours).

FIG. 33E is a flow cytometry graph showing the events (×1000) in function of TAMRA detection in the control condition (L2 in a concentration of 16 ng/μL in the Jurkat T-cell incubation of 72 hours).

FIG. 33F is a flow cytometry graph showing the events (×1000) in function of GFP detection in the control condition (L2 in a concentration of 16 ng/μL in the Jurkat T-cell incubation of 72 hours).

FIG. 33G is a flow cytometry graph showing the detection of TAMRA in function of GFP in the control condition (L2 in a concentration of 33.3 ng/μL in the Jurkat T-cell incubation of 72 hours).

FIG. 33H is a flow cytometry graph showing the events (×1000) in function of TAMRA detection in the control condition (L2 in a concentration of 33.3 ng/μL in the Jurkat T-cell incubation of 72 hours).

FIG. 331 is a flow cytometry graph showing the events (×1000) in function of GFP detection in the control condition (L2 in a concentration of 33.3 ng/μL in the Jurkat T-cell incubation of 72 hours).

FIG. 33J is a flow cytometry graph showing the detection of TAMRA in function of GFP in the control condition (L2 in a concentration of 66 ng/μL in the Jurkat T-cell incubation of 72 hours).

FIG. 33K is a flow cytometry graph showing the events (×1000) in function of TAMRA detection in the control condition (L2 in a concentration of 66 ng/μL in the Jurkat T-cell incubation of 72 hours).

FIG. 33L is a flow cytometry graph showing the events (×1000) in function of GFP detection in the control condition (L2 in a concentration of 66 ng/μL in the Jurkat T-cell incubation of 72 hours).

FIG. 34A is a flow cytometry graph showing the detection of TAMRA in function of GFP in the control condition (L3 in a concentration of 0.33 ng/μL in the Jurkat T-cell incubation of 72 hours).

FIG. 34B is a flow cytometry graph showing the events (×1000) in function of TAMRA detection in the control condition (L3 in a concentration of 0.33 ng/μL in the Jurkat T-cell incubation of 72 hours).

FIG. 34C is a flow cytometry graph showing the events (×1000) in function of GFP detection in the control condition (L3 in a concentration of 0.33 ng/μL in the Jurkat T-cell incubation of 72 hours).

FIG. 34D is a flow cytometry graph showing the detection of TAMRA in function of GFP in the control condition (L3 in a concentration of 16 ng/μL in the Jurkat T-cell incubation of 72 hours).

FIG. 34E is a flow cytometry graph showing the events (×1000) in function of TAMRA detection in the control condition (L3 in a concentration of 16 ng/μL in the Jurkat T-cell incubation of 72 hours).

FIG. 34F is a flow cytometry graph showing the events (×1000) in function of GFP detection in the control condition (L3 in a concentration of 16 ng/μL in the Jurkat T-cell incubation of 72 hours).

FIG. 34G is a flow cytometry graph showing the detection of TAMRA in function of GFP in the control condition (L3 in a concentration of 33.3 ng/μL in the Jurkat T-cell incubation of 72 hours).

FIG. 34H is a flow cytometry graph showing the events (×1000) in function of TAMRA detection in the control condition (L3 in a concentration of 33.3 ng/μL in the Jurkat T-cell incubation of 72 hours).

FIG. 341 is a flow cytometry graph showing the events (×1000) in function of GFP detection in the control condition (L3 in a concentration of 33.3 ng/μL in the Jurkat T-cell incubation of 72 hours).

FIG. 34J is a flow cytometry graph showing the detection of TAMRA in function of GFP in the control condition (L3 in a concentration of 66 ng/μL in the Jurkat T-cell incubation of 72 hours).

FIG. 34K is a flow cytometry graph showing the events (×1000) in function of TAMRA detection in the control condition (L3 in a concentration of 66 ng/μL in the Jurkat T-cell incubation of 72 hours).

FIG. 34L is a flow cytometry graph showing the events (×1000) in function of GFP detection in the control condition (L3 in a concentration of 66 ng/μL in the Jurkat T-cell incubation of 72 hours).

FIG. 35 is an image of a gel electrophoresis of insert specific primers.

FIG. 36A is a flow cytometry graph showing the detection of TAMRA in function of GFP in the control condition (no nuclease provided in the primary T-cell incubation of 48 hours).

FIG. 36B is a flow cytometry graph showing the events (×1000) in function of TAMRA detection in the control condition (no nuclease provided in the primary T-cell incubation of 48 hours).

FIG. 36C is a flow cytometry graph showing the events (×1000) in function of GFP detection in the control condition (no nuclease provided in the primary T-cell incubation of 48 hours).

FIG. 36D is a flow cytometry graph showing the detection of TAMRA in function of GFP in the control condition (L1 in a concentration of 66 ng/μL in the primary T-cell incubation of 48 hours).

FIG. 36E is a flow cytometry graph showing the events (×1000) in function of TAMRA detection in the control condition (L1 in a concentration of 66 ng/μL in the primary T-cell incubation of 48 hours).

FIG. 36F is a flow cytometry graph showing the events (×1000) in function of GFP detection in the control condition (L1 in a concentration of 66 ng/μL in the primary T-cell incubation of 48 hours).

FIG. 36G is a flow cytometry graph showing the detection of TAMRA in function of GFP in the control condition (L2 in a concentration of 66 ng/μL in the primary T-cell incubation of 48 hours).

FIG. 36H is a flow cytometry graph showing the events (×1000) in function of TAMRA detection in the control condition (L2 in a concentration of 66 ng/μL in the primary T-cell incubation of 48 hours).

FIG. 361 is a flow cytometry graph showing the events (×1000) in function of GFP detection in the control condition (L2 in a concentration of 66 ng/μL in the primary T-cell incubation of 48 hours).

FIG. 36J is a flow cytometry graph showing the detection of TAMRA in function of GFP in the control condition (L3 in a concentration of 66 ng/μL in the primary T-cell incubation of 48 hours).

FIG. 36K is a flow cytometry graph showing the events (×1000) in function of TAMRA detection in the control condition (L3 in a concentration of 66 ng/μL in the primary T-cell incubation of 48 hours).

FIG. 36L is a flow cytometry graph showing the events (×1000) in function of GFP detection in the control condition (L3 in a concentration of 66 ng/μL in the primary T-cell incubation of 48 hours).

FIG. 37A is a flow cytometry graph of analyzed peripheral T-cells obtained from control (without L1 or L2) mice and stained with APC-antiCD4 to evaluate T-cell population at the 3 h post injection stage.

FIG. 37B is a flow cytometry graph of analyzed peripheral T-cells obtained from mice treated with L1 labelled with TAMRA and stained with APC-antiCD4 to evaluate T-cell population at the 3 h post injection stage.

FIG. 37C is a flow cytometry graph of analyzed peripheral T-cells obtained from mice treated with L2 labelled with TAMRA and stained with APC-antiCD4 to evaluate T-cell population at the 3 h post injection stage.

FIG. 37D is a flow cytometry graph of analyzed peripheral T-cells obtained from control (without L1 or L2) mice and stained with APC-antiCD4 to evaluate T-cell population at the 24 h post injection stage.

FIG. 37E is a flow cytometry graph of analyzed peripheral T-cells obtained from mice treated with L1 labelled with TAMRA and stained with APC-antiCD4 to evaluate T-cell population at the 24 h post injection stage.

FIG. 37F is a flow cytometry graph of analyzed peripheral T-cells obtained from mice treated with L2 labelled with TAMRA and stained with APC-antiCD4 to evaluate T-cell population at the 24 h post injection stage.

FIG. 37G is a flow cytometry graph of analyzed peripheral T-cells obtained from control (without L1 or L2) mice and stained with APC-antiCD4 to evaluate T-cell population at the 48 h post injection stage.

FIG. 37H is a flow cytometry graph of analyzed peripheral T-cells obtained from mice treated with L1 labelled with TAMRA and stained with APC-antiCD4 to evaluate T-cell population at the 48 h post injection stage.

FIG. 371 is a flow cytometry graph of analyzed peripheral T-cells obtained from mice treated with L2 labelled with TAMRA and stained with APC-antiCD4 to evaluate T-cell population at the 48 h post injection stage.

FIG. 38A is a flow cytometry graph of T cells obtained from the mice of FIG. 37A showing the events (detection) of CD19.

FIG. 38B is a flow cytometry graph of T cells obtained from the mice of FIG. 37B showing the events (detection) of CD19.

FIG. 38C is a flow cytometry graph of T cells obtained from the mice of FIG. 37C showing the events (detection) of CD19.

FIG. 39A is a bioluminescence image of control mice (labeled as C) having received a control vehicle, mice having received L1 (labeled as L1), and mice having received L2 (labeled as L2) at 192 h post Raji injection.

FIG. 39B is a graph showing the bioluminescence for the control mice, the mice having received L1, and the mice having received L2 of FIG. 39A.

FIG. 40A is a gel showing the results of stepavidin-aptamer modifications of donor template.

FIG. 40B is a gel showing offtarget analysis for L2 treated cells.

### DETAILED DESCRIPTION

### Definitions

As used herein, the term “cell recognition domain” (or “CRD”) refers to a natural or synthetic peptide or nucleic acid domain capable of specific non-covalent association with a cell-surface antigen or receptor.

As used herein, the term “polynucleotide modifying enzyme” (or “PNME”) refers to a peptide enzyme capable of cleaving the phosphodiester backbone of a nucleic acid (e.g. DNA or RNA) or altering the identity of one or more nitrogenous bases within a nucleic acid.

As used herein, the term “endosome escape domain” (or “EE domain”) refers to a peptide sequence which, when associated with a molecular cargo, facilitates diffusion of the cargo from the endosomal compartment to the cytosol and/or alters the steady state distribution of the cargo between the endosomal compartment and in favor of the cytosol.

As used herein, the term “display domain” refers to a peptide sequence capable of specific non-covalent association with a cell-surface antigen or receptor. The display domain is incorporated in the PNME and does not disrupt the activity of the functional nuclease domain. The display domain can have a size and/or be positioned in the sequence of the PNME such that the nuclease catalytic pocket is not disrupted and retains at least 50%, at least 60%, at least 70%, preferably at least 80%, and more preferably at least 90%, of its cleaving activity. For example, the three dimensional conformation of the nuclease catalytic pocket can correspond substantially (e.g. same alpha helix and same beta sheets) to the three dimensional conformation that would be obtained without the insertion of the display domain in the PNME.

As used herein, the term “hapten” refers to a small molecule, which when combined with a larger carrier such as a protein, is capable of high affinity binding to an antibody or antibody mimetic (“hapten binding domain”). In some embodiments, the molecular weight of the organic compound is less than 500 Daltons. In some embodiments, the affinity (K_{D}) of the hapten for the hapten binding domain is less than 10⁻⁶ molar. In some embodiments, the affinity (K_{D}) of the hapten for the peptide or nucleic acid aptamer is less than 10⁻⁷ molar. In some embodiments, the affinity (K_{D}) of the hapten for the peptide or nucleic acid aptamer is less than 10⁻⁸ molar. In some embodiments, the affinity (K_{D}) of the hapten for the peptide or nucleic acid aptamer is less than 10⁻⁹ molar. As used herein, the term “linker”, “linker group” or “linker domain” means a group that can link one chemical moiety to another chemical moiety. In some embodiments, a linker is a chemical bond. In some embodiments, the linker is an organic molecule, group, polymer, or chemical moiety. In some embodiments, the linker is a cleavable linker, e.g., the linker comprises a linkage that can be cleaved upon exposure to a cleavage activity such as UV light or a hydrolase, such as a lysosomal protease. In some embodiments, the linker may comprise one or more, two or more, three or more, four or more, five or more, six or more, seven or more, eight or more, nine or more, ten or more, 20 or more, 25 or more, 30 or more, 40 or more, 50 or more amino acids. In some embodiments, the peptide linker comprises a repeat of a tri-peptide Gly-Gly-Ser, including, for example, sequence (GGS)ₙ, wherein n is at least 1, 2, 3, 4, 5, 6, 7, 8, 9, 10 or more repeats. In some embodiments, the linker can comprise at least two polyethyleneglycol (PEG) residues. In some embodiments, a PEG linker comprises three or more, four or more, five or more, six or more, seven or more, eight or more, nine or more, or ten or more PEG residues. In some embodiments, the PNME described herein comprises linkers joining two or more domains described herein, such as any combination of two or more of endosome escape domains, nuclear localization sequences, or PNME domains.

The term “tracrRNA” or “tracr sequence”, as used herein, refers to a nucleic acid with at least about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes, S. aureus, etc). tracrRNA can refer to a nucleic acid with at most about 5%, 10%, 20%, 30%, 40%, 50%, 60%, 70%, 80%, 90%, or 100% sequence identity and/or sequence similarity to a wild type exemplary tracrRNA sequence. tracrRNA may refer to a modified form of a tracrRNA that can comprise a nucleotide change such as a deletion, insertion, or substitution, variant, mutation, or chimera. A tracrRNA may refer to a nucleic acid that can be at least about 60% identical to a wild type exemplary tracrRNA sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA sequence over a stretch of at least 6 contiguous nucleotides.

As used herein, a “guide nucleic acid” refers to a nucleic acid that can hybridize to another nucleic acid. A guide nucleic acid may preferably be RNA or DNA. The guide nucleic acid may be programmed to bind specifically to a nucleic acid with a particular sequence. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called a noncomplementary strand. A guide nucleic acid may comprise a polynucleotide chain and can be called a “single guide nucleic acid”. A guide nucleic acid may comprise two polynucleotide chains and may be called a “double guide nucleic acid”. If not otherwise specified, the term “guide nucleic acid” may be inclusive, referring to both single guide nucleic acids and double guide nucleic acids. Guide nucleic acids may comprise a nucleic acid targeting segment (e.g. a crRNA) and a protein binding sequence. Guide nucleic acids may comprise a nucleic acid targeting segment (e.g. a crRNA) a protein binding sequence, and a trans-activating RNA (e.g. a tracrRNA). In some cases, a guide RNA described herein comprises a sequence of n nucleotides counting from a 1^{st} nucleotide at a 5′ end to an n^{th} nucleotide at a 3′ end, wherein one or more of the nucleotides at positions 1, 2, n−1 and n are phosphorothioate modified nucleotides. The guide nucleic acid can comprise one or more bridged nucleotides in a seed region of the guide oligonucleotide.

A guide nucleic acid may comprise a segment that can be referred to as a “nucleic acid-targeting segment”, a “nucleic acid-targeting sequence” or a “seed sequence”. In some embodiments, the sequence is 19-21 nucleotides in length. In some embodiments, the “nucleic acid-targeting segment” or the “nucleic acid-targeting sequence” comprises a crRNA. A nucleic acid-targeting segment may comprise a sub-segment that may be referred to as a “protein binding segment”, a “protein binding sequence” or a “Cas protein binding segment”.

A “host cell” generally includes an individual cell or cell culture which can be or has been a recipient for the subject vectors into which exogenous nucleic acid has been introduced, such as those described herein. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

The term a “derivative” when referring to a protein means that the protein was modified with the addition or removal of a sequence while retaining its function. The term a “functional analog” means a different sequence that performs the same function. The term a “variant” thereof means that the protein was mutated while retaining or enhancing its function.

### CAR-T Cells and Gene Editing Tools

T cells are immune cells that express a T cell receptor which is encoded by the T cell receptor a constant (TRAC) gene. T cells include CD4 T cells, CD8 T cells and NK cells and each characteristically express CD4, CD8, and CD16-CD56 respectively at their cell surface. Natural Killer (NK) cells and CD8 cytotoxic T cells are two types of immune cells that can kill target cells through similar cytotoxic mechanisms. CD4 and CD8 CAR T cell therapies have been the main focus of research and development as opposed to NK cells because NK cell immunotherapy approaches require an efficient gene transfer method in the primary NK cells that current gene editing methods do not achieve. CD16 and/or CD56 can be used as targets for specific NK cell modification. CD56 and CD16 are key clusters of differentiation for defining natural killer cells within white blood cell populations, as such they can be targeted for example by antibodies as a means to enrich, identify and characterise NK cells. Bispecific targeting can be performed by targeting both CD16 and CD56. Particularly CD56 Bright is the most active NK cell population with regards to anti cancer activity.

The present disclosure achieves a significant improvement in the efficiency of CAR-T gene editing by providing a protein based interaction to bind the cell membrane of T cells and get the genetic material and PNME internalized. The present disclosure achieves a transduction efficiency of more than 75%, preferably more than 80%, more preferably more than 90%, and even more preferably more than 95%. In contrast, traditional methods have only achieved efficiencies that are in the order of 25-30%. The efficiency that is generally reported in the literature is the efficiency post selection after a step of cell sorting, antibiotic selection, magnetic separation or the like. On the other hand, the present disclosure reports the efficiency directly without any step that artificially inflates the efficiency rate. The efficiency rate is calculated based on the total starting cell population not just the cell population that received the vector.

The presently improved efficiency means that the CAR-T cells methods described herein can be applied to NK T cells. The advantage of NK cells is that they generally offer an improved safety to the subject receiving same (i.e. a lack or minimal cytokine release syndrome and neurotoxicity), and they offer multiple mechanisms for activating cytotoxicity.

The present disclosure contemplates all types of CAR T cell receptors. These include multi-targeted CAR configurations such as:
dual CARs that co-express two different CARs in one cell,
tandem CARs containing two different scFvs in a single CAR molecule that can either be stacked in series or as a looped structure.
combinatorial CARs combine two constructs: one bears the CD3z signaling motif and the other bears the costimulatory signaling domain,
synthetic Notch (syn-Notch) receptors induce the transcription of a CAR after antigen recognition of their cognate antigen, and
inhibitory CARs (iCAR) inhibit T cell activation following antigen recognition in normal cells.

The present disclosure provides a protein complex comprising a polynucleotide-modifying enzyme domain (with a functional nuclease catalytic pocket) and an endosome escape domain, a guide oligonucleotide targeting TRAC and donor DNA. In some cases, the PNME enzymes are programmable nucleases. Such nucleases are preferably engineered to target a specific DNA or RNA sequence for cleavage. The nucleases are for example CRISPR endonucleases such as Cas9, Cas12a (Cpf1), Cas12b, Cas12c, Cas12d, Cas12e, Cas13a, Cas13b, and Cas14. For the purpose of CAR T cell therapy, the CRISPR endonucleases are preferably selected from Cas9, Cas12a (Cpf1), Cas12b, Cas12c, Cas12d, and Cas12e. In some embodiments, CRISPR endonucleases are class II CRISPR endonucleases. In some cases, CRISPR endonucleases are class II, type II, V, or VI endonucleases. In preferred embodiments for the purpose of CAR T cell production, the CRISPR endonuclease is a type II or type V Cas. In some cases, such nucleases comprise at least one nuclease deficient nuclease domain. In some embodiments, the CRIPSR endonuclease is encoded by a sequence having at least at least 75% identity, at least 78% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity to any one of SEQ ID NOs: 1, 3, 5, 7, or 9. In some embodiments, the CRIPSR endonuclease has at least 75% identity, at least 78% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity to any one of SEQ ID NOs: 2, 4, 6, 8 or 10.

TABLE 1
Exemplary nucleases

| SEQ | | |
|---|---|---|
| ID NO: | Protein | Sequence |
| 1 | spCas9 | ATGGATAAAAAATACAGCATTGGTCTGGACATTGGCACGAATAGCGTTGGTTGGGCA |
| | (nucleotide | GTGATTACCGATGAATACAAAGTCCCGTCGAAAAAATTCAAAGTGCTGGGTAACACC |
| | sequence) | GATCGCCATAGCATTAAGAAAAACCTGATCGGTGCGCTGCTGTTTGATTCTGGCGAA |
| | | ACCGCGGAAGCAACGCGTCTGAAACGTACCGCACGTCGCCGTTACACGCGCCGTAAA |
| | | AATCGTATTTGCTATCTGCAGGAAATCTTTAGCAACGAAATGGCGAAAGTCGATGAC |
| | | TCATTTTTCCACCGCCTGGAAGAATCGTTTCTGGTGGAAGAAGATAAAAAACATGAA |
| | | CGTCACCCGATTTTCGGCAATATCGTTGATGAAGTCGCGTACCATGAAAAATATCCG |
| | | ACGATTTACCACCTGCGTAAAAAACTGGTGGATTCTACCGACAAAGCCGATCTGCGC |
| | | CTGATTTATCTGGCACTGGCTCATATGATCAAATTTCGTGGTCACTTCCTGATTGAA |
| | | GGCGACCTGAACCCGGATAATAGTGACGTCGATAAACTGTTTATTCAGCTGGTGCAA |
| | | ACCTATAATCAGCTGTTCGAAGAAAACCCGATCAATGCAAGTGGTGTTGATGCGAAA |
| | | GCCATTCTGTCCGCTCGCCTGAGTAAATCCCGCCGTCTGGAAAACCTGATTGCACAG |
| | | CTGCCGGGTGAAAAGAAAAACGGTCTGTTTGGCAATCTGATCGCTCTGTCACTGGGC |
| | | CTGACGCCGAACTTTAAATCGAATTTCGACCTGGCAGAAGATGCTAAACTGCAGCTG |
| | | AGCAAAGATACCTACGATGACGATCTGGACAACCTGCTGGCGCAAATTGGCGACCAG |
| | | TATGCCGACCTGTTTCTGGCGGCCAAAAATCTGTCAGATGCCATTCTGCTGTCGGAC |
| | | ATCCTGCGCGTGAACACCGAAATCACGAAAGCGCCGCTGTCAGCCTCGATGATTAAA |
| | | CGCTACGATGAACATCACCAGGACCTGACCCTGCTGAAAGCACTGGTTCGTCAGCAA |
| | | CTGCCGGAAAAATACAAAGAAATTTTCTTTGACCAAAGTAAAAATGGTTATGCAGGC |
| | | TACATCGATGGCGGTGCTTCCCAGGAAGAATTCTACAAATTCATCAAACCGATCCTG |
| | | GAAAAAATGGATGGTACGGAAGAACTGCTGGTGAAACTGAATCGTGAAGATCTGCTG |
| | | CGTAAACAACGCACCTTTGACAACGGTAGCATTCCGCATCAGATCCACCTGGGCGAA |
| | | CTGCATGCGATTCTGCGCCGTCAGGAAGATTTTTATCCGTTCCTGAAAGACAACCGT |
| | | GAAAAAATCGAAAAAATCCTGACGTTTCGCATCCCGTATTACGTTGGTCCGCTGGCA |
| | | CGTGGTAATAGCCGCTTCGCATGGATGACCCGCAAATCTGAAGAAACCATTACGCCG |
| | | TGGAACTTTGAAGAAGTGGTTGATAAAGGCGCAAGCGCTCAGTCTTTTATCGAACGT |
| | | ATGACCAATTTCGATAAAAACCTGCCGAATGAAAAAGTGCTGCCGAAACATTCTCTG |
| | | CTGTATGAATACTTTACCGTTTACAACGAACTGACGAAAGTGAAATATGTTACCGAG |
| | | GGTATGCGCAAACCGGCGTTTCTGAGTGGCGAACAGAAAAAAGCCATTGTGGATCTG |
| | | CTGTTCAAAACCAATCGTAAAGTTACGGTCAAACAGCTGAAAGAAGATTACTTCAAG |
| | | AAAATTGAATGTTTCGACAGCGTGGAAATTTCTGGTGTTGAAGATCGTTTCAACGCC |
| | | TCTCTGGGCACCTATCATGACCTGCTGAAAATCATCAAAGACAAAGATTTTCTGGAT |
| | | AACGAAGAAAACGAAGACATTCTGGAAGATATCGTGCTGACCCTGACGCTGTTCGAA |
| | | GATCGTGAAATGATTGAAGAACGCCTGAAAACGTACGCACACCTGTTTGACGATAAA |
| | | GTTATGAAACAGCTGAAACGCCGTCGCTATACCGGTTGGGGCCGTCTGAGCCGCAAA |
| | | CTGATTAATGGTATCCGCGATAAACAATCAGGCAAAACGATTCTGGATTTCCTGAAA |
| | | TCGGACGGCTTTGCCAACCGTAATTTCATGCAGCTGATCCATGACGATTCCCTGACC |
| | | TTTAAAGAAGACATTCAGAAAGCACAAGTGTCAGGTCAAGGCGATTCGCTGCATGAA |
| | | CACATTGCGAACCTGGCCGGTTCACCGGCTATCAAAAAAGGCATCCTGCAGACCGTG |
| | | AAAGTCGTGGATGAACTGGTGAAAGTTATGGGTCGTCACAAACCGGAAAACATTGTT |
| | | ATCGAAATGGCGCGCGAAAATCAGACCACGCAAAAAGGCCAGAAAAACTCGCGTGAA |
| | | CGCATGAAACGCATTGAAGAAGGTATCAAAGAACTGGGCAGCCAGATTCTGAAAGAA |
| | | CATCCGGTCGAAAACACCCAGCTGCAAAATGAAAAACTGTACCTGTATTACCTGCAA |
| | | AATGGTCGTGACATGTATGTGGATCAGGAACTGGACATCAACCGCCTGTCTGACTAT |
| | | GATGTCGACCACATTGTGCCGCAGAGCTTTCTGAAAGACGATTCTATCGATAACAAA |
| | | GTTCTGACCCGTAGTGATAAAAACCGCGGCAAAAGCGACAATGTCCCGTCTGAAGAA |
| | | GTTGTGAAGAAAATGAAAAACTACTGGCGTCAACTGCTGAATGCGAAACTGATTACG |
| | | CAGCGTAAATTCGATAACCTGACCAAAGCGGAACGCGGCGGTCTGTCCGAACTGGAT |
| | | AAAGCCGGTTTTATCAAACGTCAACTGGTTGAAACCCGCCAGATTACGAAACATGTC |
| | | GCCCAGATCCTGGATTCACGCATGAACACGAAATACGACGAAAACGATAAACTGATC |
| | | CGTGAAGTCAAAGTGATCACCCTGAAAAGTAAACTGGTTTCCGATTTCCGTAAAGAC |
| | | TTTCAGTTCTACAAAGTCCGCGAAATTAACAATTACCATCACGCACACGATGCTTAT |
| | | CTGAATGCAGTGGTTGGTACCGCTCTGATCAAAAAATATCCGAAACTGGAAAGCGAA |
| | | TTTGTGTATGGCGATTACAAAGTCTATGACGTGCGCAAAATGATTGCGAAATCCGAA |
| | | CAGGAAATCGGCAAAGCGACCGCCAAATACTTTTTCTATTCAAACATCATGAACTTT |
| | | TTCAAAACCGAAATTACGCTGGCAAATGGTGAAATTCGTAAACGCCCGCTGATCGAA |
| | | ACCAACGGTGAAACGGGCGAAATTGTGTGGGATAAAGGCCGTGACTTCGCGACCGTT |
| | | CGCAAAGTCCTGTCGATGCCGCAAGTGAATATCGTGAAGAAAACCGAAGTGCAGACG |
| | | GGCGGTTTTAGTAAAGAATCCATCCTGCCGAAACGTAACAGCGATAAACTGATTGCG |
| | | CGCAAAAAAGATTGGGACCCGAAAAAATACGGCGGTTTTGATAGTCCGACGGTTGCA |
| | | TATTCCGTCCTGGTCGTGGCTAAAGTCGAAAAAGGTAAAAGTAAAAAACTGAAATCC |
| | | GTGAAAGAACTGCTGGGCATTACCATCATGGAACGTAGCTCTTTTGAGAAAAACCCG |
| | | ATTGACTTCCTGGAAGCCAAAGGTTACAAAGAAGTGAAAAAAGATCTGATCATCAAA |
| | | CTGCCGAAATATAGCCTGTTCGAACTGGAAAACGGCCGTAAACGCATGCTGGCATCT |
| | | GCTGGTGAACTGCAGAAAGGCAATGAACTGGCACTGCCGAGTAAATATGTTAACTTT |
| | | CTGTACCTGGCTAGCCATTATGAAAAACTGAAAGGTTCTCCGGAAGATAACGAACAG |
| | | AAACAACTGTTCGTCGAACAACATAAACACTACCTGGATGAAATCATCGAACAGATC |
| | | TCAGAATTCTCGAAACGCGTGATTCTGGCGGATGCCAATCTGGACAAAGTTCTGAGC |
| | | GCGTATAACAAACATCGTGATAAACCGATTCGCGAACAGGCCGAAAATATTATCCAC |
| | | CTGTTTACCCTGACGAACCTGGGCGCACCGGCAGCTTTTAAATACTTCGATACCACG |
| | | ATCGACCGTAAACGCTATACCTCAACGAAAGAAGTTCTGGATGCTACCCTGATTCAT |
| | | CAATCGATCACCGGTCTGTATGAAACGCGTATTGATCTGAGTCAGCTGGGCGGTGAC |
| 2 | spCas9 | MDKKYSIGLDIGTNSVGWAVITDEYKVPSKKFKVLGNTDRHSIKKNLIGALLFDSGE |
| | (protein | TAEATRLKRTARRRYTRRKNRICYLQEIFSNEMAKVDDSFFHRLEESFLVEEDKKHE |
| | sequence) | RHPIFGNIVDEVAYHEKYPTIYHLRKKLVDSTDKADLRLIYLALAHMIKFRGHFLIE |
| | | GDLNPDNSDVDKLFIQLVQTYNQLFEENPINASGVDAKAILSARLSKSRRLENLIAQ |
| | | LPGEKKNGLFGNLIALSLGLTPNFKSNFDLAEDAKLQLSKDTYDDDLDNLLAQIGDQ |
| | | YADLFLAAKNLSDAILLSDILRVNTEITKAPLSASMIKRYDEHHQDLTLLKALVRQQ |
| | | LPEKYKEIFFDQSKNGYAGYIDGGASQEEFYKFIKPILEKMDGTEELLVKLNREDLL |
| | | RKQRTFDNGSIPHQIHLGELHAILRRQEDFYPFLKDNREKIEKILTFRIPYYVGPLA |
| | | RGNSRFAWMTRKSEETITPWNFEEVVDKGASAQSFIERMTNFDKNLPNEKVLPKHSL |
| | | LYEYFTVYNELTKVKYVTEGMRKPAFLSGEQKKAIVDLLFKTNRKVTVKQLKEDYFK |
| | | KIECFDSVEISGVEDRFNASLGTYHDLLKIIKDKDFLDNEENEDILEDIVLTLTLFE |
| | | DREMIEERLKTYAHLFDDKVMKQLKRRRYTGWGRLSRKLINGIRDKQSGKTILDFLK |
| | | SDGFANRNFMQLIHDDSLTFKEDIQKAQVSGQGDSLHEHIANLAGSPAIKKGILQTV |
| | | KVVDELVKVMGRHKPENIVIEMARENQTTQKGQKNSRERMKRIEEGIKELGSQILKE |
| | | HPVENTQLQNEKLYLYYLQNGRDMYVDQELDINRLSDYDVDHIVPQSFLKDDSIDNK |
| | | VLTRSDKNRGKSDNVPSEEVVKKMKNYWRQLLNAKLITQRKFDNLTKAERGGLSELD |
| | | KAGFIKRQLVETRQITKHVAQILDSRMNTKYDENDKLIREVKVITLKSKLVSDFRKD |
| | | FQFYKVREINNYHHAHDAYLNAVVGTALIKKYPKLESEFVYGDYKVYDVRKMIAKSE |
| | | QEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTVA |
| | | YSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNEQ |
| | | KQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGG |
| 3 | IbCPF1 | ATGTCAAAGCTGGAGAAATTCACCAACTGTTATAGCCTGTCTAAGACCCTGCGCTTC |
| | (nucleotide | AAGGCAATCCCAGTGGGCAAGACACAAGAGAACATTGACAACAAACGGCTCCTGGTG |
| | sequence) | GAGGATGAGAAGAGGGCTGAAGATTACAAGGGCGTTAAGAAGCTGCTGGATAGGTAC |
| | | TATCTGTCATTCATCAACGATGTCCTCCACAGTATCAAGCTGAAGAATCTGAACAAT |
| | | TACATTTCTCTGTTCCGGAAGAAGACACGGACCGAGAAGGAGAACAAAGAGCTGGAG |
| | | AATCTGGAGATCAACCTGAGGAAAGAAATAGCTAAGGCTTTCAAAGGGAACGAGGGT |
| | | TACAAGTCCCTGTTCAAGAAAGACATTATCGAGACTATTCTGCCTGAGTTCCTGGAC |
| | | GATAAAGATGAGATCGCCCTCGTCAATTCCTTCAATGGGTTTACCACAGCCTTTACC |
| | | GGCTTCTTCGACAATAGAGAGAATATGTTCTCTGAAGAGGCCAAATCCACTAGCATC |
| | | GCCTTTCGCTGCATAAACGAGAACCTGACTAGGTACATCAGCAATATGGACATCTTT |
| | | GAGAAAGTCGATGCCATATTCGACAAACATGAGGTGCAGGAGATTAAGGAGAAGATC |
| | | CTGAACTCAGATTACGATGTCGAAGATTTCTTCGAGGGAGAGTTCTTCAACTTCGTG |
| | | CTCACACAAGAGGGCATTGATGTGTACAATGCAATCATTGGAGGGTTCGTGACAGAG |
| | | AGTGGCGAGAAGATAAAGGGCCTGAACGAGTATATCAACCTCTACAACCAGAAAACC |
| | | AAGCAGAAACTGCCTAAGTTCAAGCCACTGTACAAACAAGTGCTCTCAGATAGGGAA |
| | | AGCCTGAGCTTCTACGGTGAAGGGTATACATCAGATGAAGAAGTGCTCGAAGTGTTC |
| | | CGCAACACCCTCAATAAGAACAGTGAAATCTTCTCTTCAATCAAGAAGCTGGAGAAA |
| | | CTGTTCAAGAATTTCGATGAGTACTCCTCTGCCGGAATCTTTGTGAAGAATGGCCCT |
| | | GCAATATCCACTATTAGCAAAGACATCTTTGGCGAGTGGAACGTTATCAGGGATAAG |
| | | TGGAATGCCGAGTACGATGATATTCATCTCAAGAAGAAAGCCGTGGTTACAGAGAAA |
| | | TACGAGGATGATAGACGCAAGAGCTTTAAGAAGATTGGTAGCTTCTCTCTCGAACAG |
| | | CTGCAGGAGTACGCCGACGCTGACCTGTCAGTCGTGGAGAAACTCAAGGAGATCATA |
| | | ATCCAGAAGGTGGATGAAATCTACAAAGTGTATGGAAGCTCTGAGAAACTCTTCGAT |
| | | GCAGACTTTGTTCTGGAGAAGAGTCTGAAGAAGAACGACGCAGTGGTTGCTATCATG |
| | | AAGGACCTGCTGGATTCTGTTAAGTCTTTCGAGAATTACATTAAGGCATTCTTTGGT |
| | | GAAGGGAAGGAGACAAATAGGGACGAGAGCTTCTATGGCGACTTTGTTCTGGCCTAC |
| | | GACATCCTCCTCAAGGTTGACCACATCTATGACGCTATACGGAATTACGTTACCCAG |
| | | AAGCCCTATAGCAAAGACAAGTTCAAGCTGTATTTCCAGAATCCACAGTTTATGGGT |
| | | GGGTGGGATAAAGACAAAGAAACAGATTACAGGGCCACTATCCTGCGGTACGGCAGC |
| | | AAATACTATCTGGCTATCATGGATAAGAAGTACGCCAAATGCCTCCAGAAGATCGAC |
| | | AAGGACGACGTGAACGGTAACTACGAGAAGATCAATTACAAGCTCCTGCCAGGACCT |
| | | AACAAGATGCTGCCCAAGGTGTTCTTCTCCAAGAAATGGATGGCCTACTATAACCCA |
| | | AGCGAGGACATTCAGAAGATATACAAGAATGGGACATTCAAGAAGGGCGATATGTTC |
| | | AACCTCAACGACTGCCACAAGCTGATTGATTTCTTCAAGGATAGCATTTCTCGCTAT |
| | | CCCAAGTGGTCTAATGCATACGATTTCAACTTCAGCGAGACTGAGAAGTACAAAGAC |
| | | ATCGCTGGCTTCTACCGGGAGGTGGAAGAGCAAGGCTATAAGGTGTCATTCGAATCC |
| | | GCTTCTAAGAAGGAAGTGGATAAGCTCGTGGAAGAGGGTAAGCTGTACATGTTCCAG |
| | | ATATACAACAAAGACTTCAGCGATAAGAGCCACGGCACTCCAAACCTCCATACTATG |
| | | TATTTCAAGCTGCTGTTTGACGAGAACAACCACGGACAGATTAGGCTGTCAGGAGGC |
| | | GCAGAACTCTTCATGCGCAGAGCTTCACTGAAGAAGGAGGAACTCGTTGTCCACCCA |
| | | GCCAATAGCCCTATAGCCAATAAGAATCCAGACAATCCTAAGAAAACCACTACTCTG |
| | | TCTTACGATGTGTATAAGGATAAGAGATTCTCTGAAGATCAGTACGAACTGCACATA |
| | | CCCATTGCCATTAACAAGTGCCCTAAGAACATCTTCAAGATTAACACAGAGGTTAGA |
| | | GTGCTCCTGAAACACGACGATAACCCTTATGTTATAGGCATTGATCGCGGAGAGAGA |
| | | AACCTGCTGTACATCGTCGTGGTGGACGGCAAAGGCAACATCGTGGAACAGTACAGT |
| | | CTCAATGAAATCATTAACAATTTCAACGGAATCCGCATTAAGACCGACTACCATTCT |
| | | CTCCTCGACAAGAAGGAGAAAGAAAGGTTCGAAGCAAGACAGAATTGGACAAGTATA |
| | | GAGAATATCAAAGAACTGAAGGCTGGGTACATCTCTCAGGTTGTGCACAAGATATGT |
| | | GAGCTGGTGGAGAAGTACGACGCTGTTATCGCCCTCGAGGACCTGAATAGCGGCTTC |
| | | AAGAACTCCAGGGTGAAGGTGGAGAAGCAGGTGTATCAGAAGTTCGAGAAGATGCTG |
| | | ATCGACAAGCTCAACTATATGGTGGACAAGAAATCCAATCCTTGCGCTACTGGTGGA |
| | | GCCCTGAAGGGCTATCAAATCACCAATAAGTTCGAATCTTTCAAGTCTATGAGCACC |
| | | CAGAATGGCTTCATCTTCTACATACCCGCATGGCTGACATCCAAGATTGATCCCTCT |
| | | ACCGGATTTGTTAATCTGCTCAAGACTAAGTACACCTCTATTGCTGACTCAAAGAAG |
| | | TTCATATCATCATTTGACCGCATCATGTACGTGCCAGAAGAGGACCTGTTCGAGTTT |
| | | GCCCTGGATTACAAGAATTTCTCTCGGACTGACGCCGACTACATCAAGAAGTGGAAG |
| | | CTCTACTCTTATGGTAATCGGATTCGCATATTCCGCAATCCCAAGAAGAATAACGTG |
| | | TTCGATTGGGAGGAAGTTTGCCTCACCAGCGCTTACAAGGAGCTGTTCAATAAGTAT |
| | | GGGATTAACTACCAGCAGGGCGACATAAGAGCCCTGCTGTGCGAACAATCTGATAAG |
| | | GCATTCTATTCCTCTTTCATGGCACTGATGTCACTGATGCTGCAAATGCGCAATTCC |
| | | ATCACCGGAAGAACAGACGTGGACTTTCTGATCTCTCCTGTCAAGAACTCAGATGGC |
| | | ATCTTCTACGATTCCCGCAACTATGAAGCACAGGAGAATGCTATCCTGCCTAAGAAT |
| | | GCCGATGCAAATGGAGCCTATAACATCGCCAGAAAGGTCCTCTGGGCCATAGGACAA |
| | | TTCAAGAAAGCTGAAGATGAGAAGCTGGACAAGGTGAAGATCGCCATTTCAAACAAA |
| | | GAGTGGCTCGAATATGCTCAGACCTCAGTGAAGCAT |
| 4 | IbCPF1 | MSKLEKFTNCYSLSKTLRFKAIPVGKTQENIDNKRLLVEDEKRAEDYKGVKKLLDRY |
| | (protein | YLSFINDVLHSIKLKNLNNYISLFRKKTRTEKENKELENLEINLRKEIAKAFKGNEG |
| | sequence) | YKSLFKKDIIETILPEFLDDKDEIALVNSFNGFTTAFTGFFDNRENMFSEEAKSTSI |
| | | AFRCINENLTRYISNMDIFEKVDAIFDKHEVQEIKEKILNSDYDVEDFFEGEFFNFV |
| | | LTQEGIDVYNAIIGGFVTESGEKIKGLNEYINLYNQKTKQKLPKFKPLYKQVLSDRE |
| | | SLSFYGEGYTSDEEVLEVFRNTLNKNSEIFSSIKKLEKLFKNFDEYSSAGIFVKNGP |
| | | AISTISKDIFGEWNVIRDKWNAEYDDIHLKKKAVVTEKYEDDRRKSFKKIGSFSLEQ |
| | | LQEYADADLSVVEKLKEIIIQKVDEIYKVYGSSEKLFDADFVLEKSLKKNDAVVAIM |
| | | KDLLDSVKSFENYIKAFFGEGKETNRDESFYGDFVLAYDILLKVDHIYDAIRNYVTQ |
| | | KPYSKDKFKLYFQNPQFMGGWDKDKETDYRATILRYGSKYYLAIMDKKYAKCLQKID |
| | | KDDVNGNYEKINYKLLPGPNKMLPKVFFSKKWMAYYNPSEDIQKIYKNGTFKKGDMF |
| | | NLNDCHKLIDFFKDSISRYPKWSNAYDENFSETEKYKDIAGFYREVEEQGYKVSFES |
| | | ASKKEVDKLVEEGKLYMFQIYNKDFSDKSHGTPNLHTMYFKLLFDENNHGQIRLSGG |
| | | AELFMRRASLKKEELVVHPANSPIANKNPDNPKKTTTLSYDVYKDKRFSEDQYELHI |
| | | PIAINKCPKNIFKINTEVRVLLKHDDNPYVIGIDRGERNLLYIVVVDGKGNIVEQYS |
| | | LNEIINNFNGIRIKTDYHSLLDKKEKERFEARQNWTSIENIKELKAGYISQVVHKIC |
| | | ELVEKYDAVIALEDLNSGFKNSRVKVEKQVYQKFEKMLIDKLNYMVDKKSNPCATGG |
| | | ALKGYQITNKFESFKSMSTQNGFIFYIPAWLTSKIDPSTGFVNLLKTKYTSIADSKK |
| | | FISSFDRIMYVPEEDLFEFALDYKNFSRTDADYIKKWKLYSYGNRIRIFRNPKKNNV |
| | | FDWEEVCLTSAYKELFNKYGINYQQGDIRALLCEQSDKAFYSSFMALMSLMLQMRNS |
| | | ITGRTDVDFLISPVKNSDGIFYDSRNYEAQENAILPKNADANGAYNIARKVLWAIGQ |
| | | FKKAEDEKLDKVKIAISNKEWLEYAQTSVKH |
| 5 | M7 | ATGAACAACGGCACAAATAATTTTCAGAACTTCATCGGGATCTCAAGTTTGCAGAAA |
| | (nucleotide | ACGCTGCGCAATGCTCTGATCCCCACGGAAACCACGCAACAGTTCATCGTCAAGAAC |
| | sequence) | GGAATAATTAAAGAAGATGAGTTACGTGGCGAGAACCGCCAGATTCTGAAAGATATC |
| | | ATGGATGACTACTACCGCGGATTCATCTCTGAGACTCTGAGTTCTATTGATGACATA |
| | | GATTGGACTAGCCTGTTCGAAAAAATGGAAATTCAGCTGAAAAATGGTGATAATAAA |
| | | GATACCTTAATTAAGGAACAGACAGAGTATCGGAAAGCAATCCATAAAAAATTTGCG |
| | | AACGACGATCGGTTTAAGAACATGTTTAGCGCCAAACTGATTAGTGACATATTACCT |
| | | GAATTTGTCATCCACAACAATAATTATTCGGCATCAGAGAAAGAGGAAAAAACCCAG |
| | | GTGATAAAATTGTTTTCGCGCTTTGCGACTAGCTTTAAAGATTACTTCAAGAACCGT |
| | | GCAAATTGCTTTTCAGCGGACGATATTTCATCAAGCAGCTGCCATCGCATCGTCAAC |
| | | GACAATGCAGAGATATTCTTTTCAAATGCGCTGGTCTACCGCCGGATCGTAAAATCG |
| | | CTGAGCAATGACGATATCAACAAAATTTCGGGCGATATGAAAGATTCATTAAAAGAA |
| | | ATGAGTCTGGAAGAAATATATTCTTACGAGAAGTATGGGGAATTTATTACCCAGGAA |
| | | GGCATTAGCTTCTATAATGATATCTGTGGGAAAGTGAATTCTTTTATGAACCTGTAT |
| | | TGTCAGAAAAATAAAGAAAACAAAAATTTATACAAACTTCAGAAACTTCACAAACAG |
| | | ATTCTATGCATTGCGGACACTAGCTATGAGGTCCCGTATAAATTTGAAAGTGACGAG |
| | | GAAGTGTACCAATCAGTTAACGGCTTCCTTGATAACATTAGCAGCAAACATATAGTC |
| | | GAAAGATTACGCAAAATCGGCGATAACTATAACGGCTACAACCTGGATAAAATTTAT |
| | | ATCGTGTCCAAATTTTACGAGAGCGTTAGCCAAAAAACCTACCGCGACTGGGAAACA |
| | | ATTAATACCGCCCTCGAAATTCATTACAATAATATCTTGCCGGGTAACGGTAAAAGT |
| | | AAAGCCGACAAAGTAAAAAAAGCGGTTAAGAATGATTTACAGAAATCCATCACCGAA |
| | | ATAAATGAACTAGTGTCAAACTATAAGCTGTGCAGTGACGACAACATCAAAGCGGAG |
| | | ACTTATATACATGAGATTAGCCATATCTTGAATAACTTTGAAGCACAGGAATTGAAA |
| | | TACAATCCGGAAATTCACCTAGTTGAATCCGAGCTCAAAGCGAGTGAGCTTAAAAAC |
| | | GTGCTGGACGTGATCATGAATGCGTTTCATTGGTGTTCGGTTTTTATGACTGAGGAA |
| | | CTTGTTGATAAAGACAACAATTTTTATGCGGAACTGGAGGAGATTTACGATGAAATT |
| | | TATCCAGTAATTAGTCTGTACAACCTGGTTCGTAACTACGTTACCCAGAAACCGTAC |
| | | AGCACGAAAAAGATTAAATTGAACTTTGGAATACCGACGTTAGCAGACGGTTGGTCA |
| | | AAGTCCAAAGAGTATTCTAATAACGCTATCATACTGATGCGCGACAATCTGTATTAT |
| | | CTGGGCATCTTTAATGCGAAGAATAAACCGGACAAGAAGATTATCGAGGGTAATACG |
| | | TCAGAAAATAAGGGTGACTACAAAAAGATGATTTATAATTTGCTCCCGGGTCCCAAC |
| | | AAAATGATCCCGAAAGTTTTCTTGAGCAGCAAGACGGGGGTGGAAACGTATAAACCG |
| | | AGCGCCTATATCCTAGAGGGGTATAAACAGAATAAACATATCAAGTCTTCAAAAGAC |
| | | TTTGATATCACTTTCTGTCATGATCTGATCGACTACTTCAAAAACTGTATTGCAATT |
| | | CATCCCGAGTGGAAAAACTTCGGTTTTGATTTTAGCGACACCAGTACTTATGAAGAC |
| | | ATTTCCGGGTTTTATCGTGAGGTAGAGTTACAAGGTTACAAGATTGATTGGACATAC |
| | | ATTAGCGAAAAAGACATTGATCTGCTGCAGGAAAAAGGTCAACTGTATCTGTTCCAG |
| | | ATATATAACAAAGATTTTTCGAAAAAATCAACCGGGAATGACAACCTTCACACCATG |
| | | TACCTGAAAAATCTTTTCTCAGAAGAAAATCTTAAGGATATCGTCCTGAAACTTAAC |
| | | GGCGAAGCGGAAATCTTCTTCAGGAAGAGCAGCATAAAGAACCCAATCATTCATAAA |
| | | AAAGGCTCGATTTTAGTCAACCGTACCTACGAAGCAGAAGAAAAAGACCAGTTTGGC |
| | | AACATTCAAATTGTGCGTAAAAATATTCCGGAAAACATTTATCAGGAGCTGTACAAA |
| | | TACTTCAACGATAAAAGCGACAAAGAGCTGTCTGATGAAGCAGCCAAACTGAAGAAT |
| | | GTAGTGGGACACCACGAGGCAGCGACGAATATAGTCAAGGACTATCGCTACACGTAT |
| | | GATAAATACTTCCTTCATATGCCTATTACGATCAATTTCAAAGCCAATAAAACGGGT |
| | | TTTATTAATGATAGGATCTTACAGTATATCGCTAAAGAAAAAGACTTACATGTGATC |
| | | GGCATTGATCGGGGCGAGCGTAACCTGATCTACGTGTCCGTGATTGATACTTGTGGT |
| | | AATATAGTTGAACAGAAAAGCTTTAACATTGTAAACGGCTACGACTATCAGATAAAA |
| | | CTGAAACAACAGGAGGGCGCTAGACAGATTGCGCGGAAAGAATGGAAAGAAATTGGT |
| | | AAAATTAAAGAGATCAAAGAGGGCTACCTGAGCTTAGTAATCCACGAGATCTCTAAA |
| | | ATGGTAATCAAATACAATGCAATTATAGCGATGGAGGATTTGTCTTATGGTTTTAAA |
| | | AAAGGGCGCTTTAAGGTCGAACGGCAAGTTTACCAGAAATTTGAAACCATGCTCATC |
| | | AATAAACTCAACTATCTGGTATTTAAAGATATTTCGATTACCGAGAATGGCGGTCTC |
| | | CTGAAAGGTTATCAGCTGACATACATTCCTGATAAACTTAAAAACGTGGGTCATCAG |
| | | TGCGGCTGCATTTTTTATGTGCCTGCTGCATACACGAGCAAAATTGATCCGACCACC |
| | | GGCTTTGTGAATATCTTTAAATTTAAAGACCTGACAGTGGACGCAAAACGTGAATTC |
| | | ATTAAAAAATTTGACTCAATTCGTTATGACAGTGAAAAAAATCTGTTCTGCTTTACA |
| | | TTTGACTACAATAACTTTATTACGCAAAACACGGTCATGAGCAAATCATCGTGGAGT |
| | | GTGTATACATACGGCGTGCGCATCAAACGTCGCTTTGTGAACGGCCGCTTCTCAAAC |
| | | GAAAGTGATACCATTGACATAACCAAAGATATGGAGAAAACGTTGGAAATGACGGAC |
| | | ATTAACTGGCGCGATGGCCACGATCTTCGTCAAGACATTATAGATTATGAAATTGTT |
| | | CAGCACATATTCGAAATTTTCCGTTTAACAGTGCAAATGCGTAACTCCTTGTCTGAA |
| | | CTGGAGGACCGTGATTACGATCGTCTCATTTCACCTGTACTGAACGAAAATAACATT |
| | | TTTTATGACAGCGCGAAAGCGGGGGATGCACTTCCTAAGGATGCCGATGCAAATGGT |
| | | GCGTATTGTATTGCATTAAAAGGGTTATATGAAATTAAACAAATTACCGAAAATTGG |
| | | AAAGAAGATGGTAAATTTTCGCGCGATAAACTCAAAATCAGCAATAAAGATTGGTTC |
| | | GACTTTATCCAGAATAAGCGCTATCTCTAA |
| 6 | M7 (protein | MNNGTNNFQNFIGISSLQKTLRNALIPTETTQQFIVKNGIIKEDELRGENRQILKDI |
| | sequence) | MDDYYRGFISETLSSIDDIDWTSLFEKMEIQLKNGDNKDTLIKEQTEYRKAIHKKFA |
| | | NDDRFKNMFSAKLISDILPEFVIHNNNYSASEKEEKTQVIKLFSRFATSFKDYFKNR |
| | | ANCFSADDISSSSCHRIVNDNAEIFFSNALVYRRIVKSLSNDDINKISGDMKDSLKE |
| | | MSLEEIYSYEKYGEFITQEGISFYNDICGKVNSFMNLYCQKNKENKNLYKLQKLHKQ |
| | | ILCIADTSYEVPYKFESDEEVYQSVNGELDNISSKHIVERLRKIGDNYNGYNLDKIY |
| | | IVSKFYESVSQKTYRDWETINTALEIHYNNILPGNGKSKADKVKKAVKNDLQKSITE |
| | | INELVSNYKLCSDDNIKAETYIHEISHILNNFEAQELKYNPEIHLVESELKASELKN |
| | | VLDVIMNAFHWCSVFMTEELVDKDNNFYAELEEIYDEIYPVISLYNLVRNYVTQKPY |
| | | STKKIKLNFGIPTLADGWSKSKEYSNNAIILMRDNLYYLGIFNAKNKPDKKIIEGNT |
| | | SENKGDYKKMIYNLLPGPNKMIPKVFLSSKTGVETYKPSAYILEGYKQNKHIKSSKD |
| | | FDITFCHDLIDYFKNCIAIHPEWKNFGFDFSDTSTYEDISGFYREVELQGYKIDWTY |
| | | ISEKDIDLLQEKGQLYLFQIYNKDFSKKSTGNDNLHTMYLKNLFSEENLKDIVLKLN |
| | | GEAEIFFRKSSIKNPIIHKKGSILVNRTYEAEEKDQFGNIQIVRKNIPENIYQELYK |
| | | YFNDKSDKELSDEAAKLKNVVGHHEAATNIVKDYRYTYDKYFLHMPITINFKANKTG |
| | | FINDRILQYIAKEKDLHVIGIDRGERNLIYVSVIDTCGNIVEQKSFNIVNGYDYQIK |
| | | LKQQEGARQIARKEWKEIGKIKEIKEGYLSLVIHEISKMVIKYNAIIAMEDLSYGFK |
| | | KGRFKVERQVYQKFETMLINKLNYLVFKDISITENGGLLKGYQLTYIPDKLKNVGHQ |
| | | CGCIFYVPAAYTSKIDPTTGFVNIFKFKDLTVDAKREFIKKFDSIRYDSEKNLFCFT |
| | | FDYNNFITQNTVMSKSSWSVYTYGVRIKRRFVNGRFSNESDTIDITKDMEKTLEMTD |
| | | INWRDGHDLRQDIIDYEIVQHIFEIFRLTVQMRNSLSELEDRDYDRLISPVLNENNI |
| | | FYDSAKAGDALPKDADANGAYCIALKGLYEIKQITENWKEDGKFSRDKLKISNKDWF |
| | | DFIQNKRYL |
| 7 | saCas9 | ATGAAAAGGAACTACATTCTGGGGCTGGACATCGGGATTACAAGCGTGGGGTATGGG |
| | (nucleotide | ATTATTGACTATGAAACAAGGGACGTGATCGACGCAGGCGTCAGACTGTTCAAGGAG |
| | sequence) | GCCAACGTGGAAAACAATGAGGGACGGAGAAGCAAGAGGGGAGCCAGGCGCCTGAAA |
| | | CGACGGAGAAGGCACAGAATCCAGAGGGTGAAGAAACTGCTGTTCGATTACAACCTG |
| | | CTGACCGACCATTCTGAGCTGAGTGGAATTAATCCTTATGAAGCCAGGGTGAAAGGC |
| | | CTGAGTCAGAAGCTGTCAGAGGAAGAGTTTTCCGCAGCTCTGCTGCACCTGGCTAAG |
| | | CGCCGAGGAGTGCATAACGTCAATGAGGTGGAAGAGGACACCGGCAACGAGCTGTCT |
| | | ACAAAGGAACAG |
| | | ATCTCACGCAATAGCAAAGCTCTGGAAGAGAAGTATGTCGCAGAGCTACAGCTGGAA |
| | | CGGCTGAAGAAAGATGGCGAGGTGAGAGGGTCAATTAATAGGTTCAAGACAAGCGAC |
| | | TACGTCAAAGAAGCCAAGCAGCTGCTGAAAGTGCAGAAGGCTTACCACCAGCTGGAT |
| | | CAGAGCTTCATCGATACTTATATCGACCTGCTGGAGACTCGGAGAACCTACTATGAG |
| | | GGACCAGGAGAAGGGAGCCCCTTCGGATGGAAAGACATCAAGGAATGGTACGAGATG |
| | | CTGATGGGACATTGCACCTATTTTCCAGAAGAGCTGAGAAGCGTCAAGTACGCTTAT |
| | | AACGCAGATCTGTACAACGCCCTGAATGACCTGAACAACCTGGTCATCACCAGGGAT |
| | | GAAAACGAGAAACTGGAATACTATGAGAAGTTCCAGATCATCGAAAACGTGTTTAAG |
| | | CAGAAGAAAAAGCCTACACTGAAACAGATTGCTAAGGAGATCCTGGTCAACGAAGAG |
| | | GACATCAAGGGCTACCGGGTGACAAGCACTGGAAAACCAGAGTTCACCAATCTGAAA |
| | | GTGTATCACGATATTAAGGACATCACAGCACGGAAAGAAATCATTGAGAACGCCGAA |
| | | CTGCTGGATCAGATTGCTAAGATCCTGACTATCTACCAGAGTTCCGAGGACATCCAG |
| | | GAAGAGCTGACTAACCTGAACAGCGAGCTGACCCAGGAAGAGATCGAACAGATTAGT |
| | | AATCTGAAGGGGTACACCGGAACACACAACCTGTCCCTGAAAGCTATCAATCTGATT |
| | | CTGGATGAGCTGTGGCATACAAACGACAATCAGATTGCAATCTTTAACCGGCTGAAG |
| | | CTGGTACCAAAAAAGGTGGACCTGAGTCAGCAGAAAGAGATCCCAACCACACTGGTG |
| | | GACGATTTCATTCTGTCACCCGTGGTCAAGCGGAGCTTCATCCAGAGCATCAAAGTG |
| | | ATCAACGCCATCATCAAGAAGTACGGCCTGCCCAATGATATCATTATCGAGCTGGCT |
| | | AGGGAGAAGAACAGCAAGGACGCACAGAAGATGATCAATGAGATGCAGAAACGAAAC |
| | | CGGCAGACCAATGAACGCATTGAAGAGATTATCCGAACTACCGGGAAAGAGAACGCA |
| | | AAGTACCTGATTGAAAAAATCAAGCTGCACGATATGCAGGAGGGAAAGTGTCTGTAT |
| | | TCTCTGGAGGCCATCCCCCTGGAGGACCTGCTGAACAATCCATTCAACTACGAGGTC |
| | | GATCATATTATCCCCAGAAGCGTGTCCTTCGACAATTCCTTTAACAACAAGGTGCTG |
| | | GTCAAGCAGGAAGAGAACTCTAAAAAGGGCAATAGGACTCCTTTCCAGTACCTGTCT |
| | | AGTTCAGATTCCAAGATCTCTTACGAAACCTTTAAAAAGCACATTCTGAATCTGGCC |
| | | AAAGGAAAGGGCCGCATCAGCAAGACCAAAAAGGAGTACCTGCTGGAAGAGCGGGAC |
| | | ATCAACAGATTCTCCGTCCAGAAGGATTTTATTAACCGGAATCTGGTGGACACAAGA |
| | | TACGCTACTCGCGGCCTGATGAATCTGCTGCGATCCTATTTCCGGGTGAACAATCTG |
| | | GATGTGAAAGTCAAGTCCATCAACGGCGGGTTCACATCTTTTCTGAGGCGCAAATGG |
| | | AAGTTTAAAAAGGAGCGCAACAAAGGGTACAAGCACCATGCCGAAGATGCTCTGATT |
| | | ATCGCAAATGCCGACTTCATCTTTAAGGAGTGGAAAAAGCTGGACAAAGCCAAGAAA |
| | | GTGATGGAGAACCAGATGTTCGAAGAGAAGCAGGCCGAATCTATGCCCGAAATCGAG |
| | | ACAGAACAGGAGTACAAGGAGATTTTCATCACTCCTCACCAGATCAAGCATATCAAG |
| | | GATTTCAAGGACTACAAGTACTCTCACCGGGTGGATAAAAAGCCCAACAGAGAGCTG |
| | | ATCAATGACACCCTGTATAGTACAAGAAAAGACGATAAGGGGAATACCCTGATTGTG |
| | | AACAATCTGAACGGACTGTACGACAAAGATAATGACAAGCTGAAAAAGCTGATCAAC |
| | | AAAAGTCCCGAGAAGCTGCTGATGTACCACCATGATCCTCAGACATATCAGAAACTG |
| | | AAGCTGATTATGGAGCAGTACGGCGACGAGAAGAACCCACTGTATAAGTACTATGAA |
| | | GAGACTGGGAACTACCTGACCAAGTATAGCAAAAAGGATAATGGCCCCGTGATCAAG |
| | | AAGATCAAGTACTATGGGAACAAGCTGAATGCCCATCTGGACATCACAGACGATTAC |
| | | CCTAACAGTCGCAACAAGGTGGTCAAGCTGTCACTGAAGCCATACAGATTCGATGTC |
| | | TATCTGGACAACGGCGTGTATAAATTTGTGACTGTCAAGAATCTGGATGTCATCAAA |
| | | AAGGAGAACTACTATGAAGTGAATAGCAAGTGCTACGAAGAGGCTAAAAAGCTGAAA |
| | | AAGATTAGCAACCAGGCAGAGTTCATCGCCTCCTTTTACAACAACGACCTGATTAAG |
| | | ATCAATGGCGAACTGTATAGGGTCATCGGGGTGAACAATGATCTGCTGAACCGCATT |
| | | GAAGTGAATATGATTGACATCACTTACCGAGAGTATCTGGAAAACATGAATGATAAG |
| | | CGCCCCCCTCGAATTATCAAAACAATCGCCTCTAAGACTCAGAGTATCAAAAAGTAC |
| | | TCAACCGACATTCTGGGAAACCTGTATGAGGTGAAGAGCAAAAAGCACCCTCAGATT |
| | | ATCAAAAAGGGCTAA |
| 8 | (protein | MKRNYILGLDIGITSVGYGIIDYETRDVIDAGVRLFKEANVENNEGRRSKRGARRLK |
| | sequence) | RRRRHRIQRVKKLLFDYNLLTDHSELSGINPYEARVKGLSQKLSEEEFSAALLHLAK |
| | | RRGVHNVNEVEEDTGNELSTKEQISRNSKALEEKYVAELQLERLKKDGEVRGSINRF |
| | | KTSDYVKEAKQLLKVQKAYHQLDQSFIDTYIDLLETRRTYYEGPGEGSPFGWKDIKE |
| | | WYEMLMGHCTYFPEELRSVKYAYNADLYNALNDLNNLVITRDENEKLEYYEKFQIIE |
| | | NVFKQKKKPTLKQIAKEILVNEEDIKGYRVTSTGKPEFTNLKVYHDIKDITARKEII |
| | | ENAELLDQIAKILTIYQSSEDIQEELTNLNSELTQEEIEQISNLKGYTGTHNLSLKA |
| | | INLILDELWHTNDNQIAIFNRLKLVPKKVDLSQQKEIPTTLVDDFILSPVVKRSFIQ |
| | | SIKVINAIIKKYGLPNDIIIELAREKNSKDAQKMINEMQKRNRQTNERIEEIIRTTG |
| | | KENAKYLIEKIKLHDMQEGKCLYSLEAIPLEDLLNNPFNYEVDHIIPRSVSFDNSEN |
| | | NKVLVKQEENSKKGNRTPFQYLSSSDSKISYETFKKHILNLAKGKGRISKTKKEYLL |
| | | EERDINRFSVQKDFINRNLVDTRYATRGLMNLLRSYFRVNNLDVKVKSINGGFTSFL |
| | | RRKWKFKKERNKGYKHHAEDALIIANADFIFKEWKKLDKAKKVMENQMFEEKQAESM |
| | | PEIETEQEYKEIFITPHQIKHIKDFKDYKYSHRVDKKPNRELINDTLYSTRKDDKGN |
| | | TLIVNNLNGLYDKDNDKLKKLINKSPEKLLMYHHDPQTYQKLKLIMEQYGDEKNPLY |
| | | KYYEETGNYLTKYSKKDNGPVIKKIKYYGNKLNAHLDITDDYPNSRNKVVKLSLKPY |
| | | RFDVYLDNGVYKFVTVKNLDVIKKENYYEVNSKCYEEAKKLKKISNQAEFIASFYNN |
| | | DLIKINGELYRVIGVNNDLLNRIEVNMIDITYREYLENMNDKRPPRIIKTIASKTQS |
| | | IKKYSTDILGNLYEVKSKKHPQIIKKG |
| 9 | asCPF1 | ATGACCCAGTTCGAGGGGTTTACCAATCTGTATCAAGTGAGCAAGACGCTGCGCTTT |
| | (nucleotide | GAACTGATCCCACAGGGAAAAACCTTAAAACATATTCAAGAGCAGGGCTTTATCGAA |
| | sequence) | GAAGATAAGGCCCGTAATGACCATTACAAAGAGTTAAAGCCGATTATTGATCGTATC |
| | | TACAAGACCTATGCGGACCAGTGCTTACAATTGGTACAGCTTGATTGGGAGAACCTC |
| | | TCTGCCGCCATCGATTCCTATCGTAAAGAAAAAACTGAAGAAACGCGCAACGCCCTG |
| | | ATTGAAGAGCAGGCCACCTATCGTAACGCGATTCATGACTATTTTATTGGCCGTACG |
| | | GACAATCTGACGGACGCGATCAACAAGCGCCATGCGGAGATTTACAAAGGACTGTTT |
| | | AAGGCTGAACTGTTCAATGGTAAGGTCCTTAAACAGCTTGGGACCGTCACAACGACG |
| | | GAACATGAAAACGCGTTATTACGTAGCTTCGACAAGTTTACCACGTATTTCTCCGGC |
| | | TTTTACGAAAATCGCAAAAACGTTTTCAGTGCCGAGGATATTTCCACTGCTATCCCT |
| | | CATCGCATTGTGCAAGACAACTTCCCAAAATTCAAAGAAAATTGTCATATCTTCACC |
| | | CGCTTAATCACCGCTGTACCGTCCCTGCGTGAGCATTTCGAAAACGTGAAAAAGGCC |
| | | ATTGGTATCTTCGTGTCTACTTCGATTGAGGAGGTATTTTCCTTTCCATTCTATAAT |
| | | CAGCTGCTGACCCAGACCCAAATTGATCTGTACAACCAGCTGCTTGGCGGTATTTCT |
| | | CGTGAAGCAGGAACCGAAAAAATCAAAGGGTTGAACGAGGTGCTTAATCTGGCAATC |
| | | CAGAAAAATGATGAAACCGCCCACATCATTGCTTCGTTACCTCATCGTTTTATCCCG |
| | | TTGTTCAAGCAAATTTTAAGTGATCGCAATACGCTGTCGTTTATTCTGGAAGAATTC |
| | | AAAAGTGATGAAGAGGTAATTCAGTCGTTTTGCAAATATAAAACCCTGTTACGTAAC |
| | | GAAAATGTCCTGGAAACAGCCGAGGCTTTGTTTAACGAACTGAATAGCATTGACCTG |
| | | ACGCATATCTTTATTAGCCACAAAAAATTAGAGACCATCTCATCAGCTCTGTGCGAT |
| | | CATTGGGATACACTGCGCAATGCGCTGTATGAACGTCGTATTTCGGAATTGACTGGC |
| | | AAAATCACTAAAAGCGCGAAAGAGAAAGTACAGCGCTCGCTTAAACATGAAGATATC |
| | | AACCTGCAGGAGATCATCAGCGCCGCGGGTAAAGAACTGTCGGAGGCATTTAAACAG |
| | | AAGACGAGCGAGATTCTGTCCCACGCACATGCCGCCTTAGACCAGCCGCTCCCGACC |
| | | ACTCTGAAGAAACAGGAAGAGAAAGAAATCCTTAAAAGTCAACTGGACAGTTTACTG |
| | | GGTCTCTATCATCTGCTGGATTGGTTTGCGGTAGACGAAAGCAATGAAGTGGATCCG |
| | | GAGTTTAGTGCCCGTCTGACAGGAATCAAGCTGGAAATGGAGCCTTCGCTTAGCTTC |
| | | TACAACAAAGCCCGCAATTATGCCACGAAAAAACCCTATAGTGTCGAAAAATTTAAA |
| | | CTCAACTTTCAAATGCCGACCCTTGCGTCGGGCTGGGATGTCAACAAAGAAAAAAAC |
| | | AACGGAGCTATTCTGTTCGTTAAAAATGGTCTGTACTACCTGGGCATCATGCCGAAA |
| | | CAGAAAGGTCGCTACAAAGCCCTTTCGTTCGAGCCCACGGAAAAAACAAGCGAAGGC |
| | | TTCGACAAAATGTACTACGATTACTTTCCGGATGCAGCAAAAATGATCCCGAAATGT |
| | | TCCACACAGCTGAAAGCCGTTACAGCACATTTTCAGACGCACACCACCCCCATCTTA |
| | | CTGTCCAACAATTTTATTGAACCGCTGGAGATTACTAAAGAAATTTATGATTTGAAC |
| | | AATCCGGAAAAAGAGCCAAAAAAGTTTCAAACCGCCTACGCTAAAAAAACCGGGGAT |
| | | CAGAAAGGGTACCGCGAAGCGTTGTGCAAGTGGATTGATTTCACCCGCGATTTTCTC |
| | | AGTAAATATACCAAGACTACCTCGATTGACCTGAGCTCACTGCGCCCGAGCTCTCAA |
| | | TATAAGGATTTGGGTGAGTACTATGCTGAATTAAACCCTTTATTGTACCACATTTCT |
| | | TTTCAGCGCATCGCCGAAAAGGAAATTATGGACGCAGTCGAAACCGGGAAACTGTAC |
| | | CTGTTCCAGATCTATAATAAGGACTTCGCCAAAGGACATCATGGCAAACCGAACCTG |
| | | CACACCCTTTACTGGACCGGGCTTTTCTCTCCGGAAAATTTGGCGAAAACCTCGATC |
| | | AAGCTTAACGGTCAAGCTGAGCTGTTTTACCGTCCAAAATCCCGCATGAAGCGCATG |
| | | GCGCATCGTTTAGGTGAAAAAATGCTGAATAAGAAACTGAAAGATCAGAAAACCCCT |
| | | ATCCCGGATACCCTCTACCAGGAACTGTATGATTACGTGAACCATCGTCTCTCGCAT |
| | | GACCTGTCAGACGAAGCGCGTGCGTTACTGCCCAATGTAATCACAAAAGAAGTTTCG |
| | | CATGAAATTATTAAAGATCGTCGTTTTACATCTGATAAATTCTTTTTTCATGTTCCG |
| | | ATCACCCTCAACTATCAGGCCGCAAACAGTCCAAGTAAGTTTAACCAGCGCGTTAAT |
| | | GCTTACCTGAAGGAACATCCGGAGACTCCGATTATTGGAATTGATCGCGGTGAACGT |
| | | AATTTGATCTATATCACTGTGATCGATAGTACCGGTAAGATTCTGGAGCAGCGCAGC |
| | | TTGAACACAATTCAACAGTTTGATTATCAGAAAAAATTAGACAACCGCGAAAAAGAG |
| | | CGCGTGGCTGCCCGTCAGGCGTGGTCTGTTGTCGGTACCATTAAAGATCTGAAGCAG |
| | | GGCTATCTTTCTCAGGTTATTCACGAAATTGTAGATCTGATGATCCATTATCAGGCG |
| | | GTTGTTGTGTTGGAGAATCTCAATTTCGGTTTTAAGAGTAAGCGCACAGGCATCGCT |
| | | GAAAAAGCAGTTTATCAGCAGTTTGAAAAAATGCTGATCGACAAATTGAACTGTTTA |
| | | GTTCTCAAAGATTACCCAGCGGAAAAGGTGGGCGGAGTGCTGAATCCGTACCAATTA |
| | | ACGGATCAATTCACTTCCTTCGCAAAGATGGGTACCCAAAGCGGCTTTCTGTTCTAT |
| | | GTGCCGGCCCCGTATACCTCGAAAATCGATCCACTGACGGGCTTCGTAGATCCGTTC |
| | | GTGTGGAAAACCATTAAAAATCATGAAAGTCGTAAACATTTTCTCGAAGGCTTCGAC |
| | | TTCCTGCACTACGACGTGAAAACTGGCGATTTCATTCTGCATTTTAAAATGAACCGC |
| | | AACCTTTCGTTTCAGCGCGGTCTGCCGGGCTTTATGCCGGCTTGGGACATTGTTTTT |
| | | GAGAAAAATGAAACCCAGTTTGATGCTAAAGGCACTCCTTTCATCGCCGGTAAACGC |
| | | ATCGTACCTGTGATTGAAAACCATCGTTTTACAGGGCGTTACCGTGATTTATACCCG |
| | | GCGAACGAATTGATCGCGCTGCTGGAGGAAAAGGGCATCGTTTTCCGTGACGGCTCC |
| | | AATATTCTGCCGAAATTACTGGAAAACGACGATTCACACGCAATTGATACCATGGTC |
| | | GCACTGATTCGCTCAGTCTTACAGATGCGTAACTCTAATGCAGCCACAGGAGAAGAT |
| | | TATATTAATTCGCCAGTCCGCGATTTGAACGGTGTTTGCTTCGACAGCCGTTTTCAG |
| | | AATCCTGAATGGCCGATGGACGCTGATGCCAACGGAGCTTATCATATCGCCCTGAAA |
| | | GGCCAGCTCCTGCTGAACCACCTGAAGGAAAGCAAAGATCTGAAATTGCAGAACGGC |
| | | ATTAGCAACCAGGACTGGTTAGCATACATCCAGGAACTGCGTAAC |
| 10 | asCPF1 | MTQFEGFTNLYQVSKTLRFELIPQGKTLKHIQEQGFIEEDKARNDHYKELKPIIDRI |
| | (protein | YKTYADQCLQLVQLDWENLSAAIDSYRKEKTEETRNALIEEQATYRNAIHDYFIGRT |
| | sequence) | DNLTDAINKRHAEIYKGLFKAELFNGKVLKQLGTVTTTEHENALLRSFDKFTTYFSG |
| | | FYENRKNVFSAEDISTAIPHRIVQDNFPKFKENCHIFTRLITAVPSLREHFENVKKA |
| | | IGIFVSTSIEEVFSFPFYNQLLTQTQIDLYNQLLGGISREAGTEKIKGLNEVLNLAI |
| | | QKNDETAHIIASLPHRFIPLFKQILSDRNTLSFILEEFKSDEEVIQSFCKYKTLLRN |
| | | ENVLETAEALFNELNSIDLTHIFISHKKLETISSALCDHWDTLRNALYERRISELTG |
| | | KITKSAKEKVQRSLKHEDINLQEIISAAGKELSEAFKQKTSEILSHAHAALDQPLPT |
| | | TLKKQEEKEILKSQLDSLLGLYHLLDWFAVDESNEVDPEFSARLTGIKLEMEPSLSF |
| | | YNKARNYATKKPYSVEKFKLNFQMPTLASGWDVNKEKNNGAILFVKNGLYYLGIMPK |
| | | QKGRYKALSFEPTEKTSEGFDKMYYDYFPDAAKMIPKCSTQLKAVTAHFQTHTTPIL |
| | | LSNNFIEPLEITKEIYDLNNPEKEPKKFQTAYAKKTGDQKGYREALCKWIDFTRDEL |
| | | SKYTKTTSIDLSSLRPSSQYKDLGEYYAELNPLLYHISFQRIAEKEIMDAVETGKLY |
| | | LFQIYNKDFAKGHHGKPNLHTLYWTGLFSPENLAKTSIKLNGQAELFYRPKSRMKRM |
| | | AHRLGEKMLNKKLKDQKTPIPDTLYQELYDYVNHRLSHDLSDEARALLPNVITKEVS |
| | | HEIIKDRRFTSDKFFFHVPITLNYQAANSPSKFNQRVNAYLKEHPETPIIGIDRGER |
| | | NLIYITVIDSTGKILEQRSLNTIQQFDYQKKLDNREKERVAARQAWSVVGTIKDLKQ |
| | | GYLSQVIHEIVDLMIHYQAVVVLENLNFGFKSKRTGIAEKAVYQQFEKMLIDKLNCL |
| | | VLKDYPAEKVGGVLNPYQLTDQFTSFAKMGTQSGFLFYVPAPYTSKIDPLTGFVDPF |
| | | VWKTIKNHESRKHFLEGFDFLHYDVKTGDFILHFKMNRNLSFQRGLPGFMPAWDIVE |
| | | EKNETQFDAKGTPFIAGKRIVPVIENHRFTGRYRDLYPANELIALLEEKGIVERDGS |
| | | NILPKLLENDDSHAIDTMVALIRSVLQMRNSNAATGEDYINSPVRDLNGVCFDSRFQ |
| | | NPEWPMDADANGAYHIALKGQLLLNHLKESKDLKLQNGISNQDWLAYIQELRN |

In some aspects, the PNME of the present disclosure is linked to cationic peptides adapted to bind the negatively charged cell membrane. The cationic peptide confers the protein complex a non covalent positive charge. The physical adsorption of the cationic peptide to the surface of oppositely charged proteins on a cell membrane enables cationic transfection of cell membranes. Accordingly in such aspects, the cell targeting and internalization is not specific to T cells. However, cationic peptides can be used in vitro when the T cells are the cells in culture and there is no specific cell targeting needed. Cationic peptides can have a length of 10 to 20 amino acids, and contain repeating or non-repeating positively charged amino acids such as R and L. In one example, the cationic peptide is SEQ ID NO: 11 which is RRRRRRRLLLLLLLL. On the other hand, in vivo applications of CAR T gene editing generally require the specific targeting of the T cells. Accordingly, in other aspects, the PNME is modified to have a domain that targets and binds T cells or a specific subtype of T cells.

Accordingly, in some aspects, the present disclosure provides for a PNME that is modified and comprises a cell recognition domain, an endosome escape domain, and a polynucleotide-modifying enzyme domain, with the endosome escape domain being covalently coupled to the cell recognition domain. The cell recognition domain targets a T cell marker such as CD4, CD8, CD16 or CD56.

The cell recognition domain can be a natural or synthetic peptide or nucleic acid domain capable of specific non-covalent association with a cell-surface antigen or receptor. The cell recognition domain can bind to an epitope of the cell-surface antigen or receptor. In some embodiments, the cell recognition domain is an antibody or antigen-binding fragment thereof, or an antibody mimetic. Antibodies include camelid antibodies. Antigen-binding fragments include Fab fragments, Fab′ fragments, F(ab′)₂ fragments, fragments produced by Fab expression libraries, Fd fragments, Fv fragments, disulfide linked Fv (dsFv) domains, single chain antibody (e.g. scFv) domains, VHH domains, or single domain antibodies. Antibody mimetics are non-antibody derived peptides or nucleic acids that bind with similar affinity to antibodies and include affibodies, affilins, affimers, affitins, alphabodies, anticalins, atrimers, avimers, aptamers, DARPins, fynomers, knottins, Kunitz domain peptides, monobodies, nanoCLAMPs, and linear peptides of 6-20 amino acids. Suitable antibody mimetics can be derived by mammalian cell, bacterial cell, or bacteriophage display by systematic evolution of ligands by exponential enrichment (SELEX™) or DNA encoded library approaches involving e.g. immobilization of a given antigen on a surface followed by binding selection. In some cases, the cell recognition domain is an aptamer oligonucleotide, such as a polyribonucleotide or a polydeoxyribonucleotide; design. Such oligonucleotide aptamers can comprise non-canonical nucleotides, such as 2′-OMe, 2′-F, or 4′-S nucleotides, 2′-FANAs, HNAs, or locked nucleic acid residues. In some embodiments, the cell recognition domain comprises a chemical ligand with a molecular weight of less than about 800 Da. Such ligands include small-molecule ligands of cell-surface small-molecule receptors such as folate (which binds to the folate receptor), piperidine carboxyamides (which bind to FSHR), phenylpyrazole or thienopyrimidine compounds (which bind to LHR), cinacalcet or analogs (which bind to CRF1) or nitro-bezoxadiazole compounds (which bind to EGFR). Such ligands also include protein ligands of cell-surface receptors such as IL2 (which binds to IL2alpha receptor), EGF (which binds to EGFR), or HFG (which binds to HFGR). In some cases, the cell recognition domain does not directly associate with a cell surface antigen but rather is capable of binding a protein ligand that is selective for a cell-surface receptor or carbohydrate. In some cases, the cell recognition domain comprises a protein ligand that is selective for a cell-surface receptor or carbohydrate. In some cases, the protein ligand that is selective for a cell-surface receptor or carbohydrate comprises 5-15 amino acids in length. In some cases, the protein ligand is a peptide growth hormone. In some cases, the protein ligand has a globular or cyclical structure.

In some aspects, the PNME of the present disclosure has been modified to incorporate a display domain to achieve a display on the exterior surface of the PNME that targets a T cell marker such as CD4, CD8, CD16 or CD56. The PNME can therefore, in such aspects, be considered a single protein delivery platform. In some embodiments, a “single protein” means that an entire sequence of the single protein is contained between the N and C terminus and that no linkage or fusion is performed at the N or C terminus. In some embodiments, the display domains of the present disclosure are positioned at least 25 amino acids after the N terminus or at least 25 amino acids before the C terminus of the polynucleotide-modifying enzyme. In some embodiments, the display domain is positioned at least 30, at least 40, at least 50, at least 75 or at least 100 amino acids after the N terminus, or at least 30, at least 40, at least 50, at least 75 or at least 100 amino acids before the C terminus. Cell penetrating peptides have been used as a platform for the delivery of biomolecules. However, generally, cell penetrating peptides do not have the same specificity and success as delivery platforms that include immunoglobulin approaches. An exemplary immunoglobulin approach can be that the antibody or antibody mimetic is first screened against a defined biological target such as a receptor and then validated with respect to target recognition. CRISPR proteins have been fused with peptides such as RGB, SV40NLS at the C and N terminal of the protein, or associated by charge to CRISPR RNP affecting non-specific entry to cells. It is preferable in order to influence organ tropism or preferential tissue accumulation that receptor specific binding should be a feature of the PNME which thus acts as a cell penetrating peptide. In some embodiments, the cell recognition domain is a peptidic sequence of SEQ ID NO: 12: QQYYSYRT which targets CD4.

In some aspect, the PNME of the present disclosure was modified to include an antigen binding domain, in a loop that is positioned on an external surface of the PNME. The PNME of this aspect is also a single protein since the antigen binding domain is inserted in an external loop between the N and C terminus of the PNME. It was surprisingly found that large a domain (e.g. more than 20 amino acids, more than 50 amino acids, more than 100 amino acids, from 100 to 200 amino acids or from 136 to 156 amino acids) can be incorporated in a loop of the PNME without disrupting the folding of the catalytic active nuclease pocket of the PNME. Indeed, the antigen binding domain is selected from Fab, single-domain antibody (sdAb), V_{HH}, or camelid antibody domain, positioned in a loop on an external surface of the polynucleotide-modifying enzyme. A linker domain is preferably included upstream of the antigen binding domain which helps the three dimensional conformation of the PNME to maintain its catalytic activity while providing a specific targeting to a desired cell type. In some embodiments, the linker domain has a size of from 0 to 30, from 8 to 30, from 10 to 30, from 12 to 28, from 16 to 25 or from 18 to 23 amino acids. In some embodiments, the antigen binding domain targets a T cell marker however, the antigen binding domain may target any other cell type or cell receptor. For example, the antigen binding domain can target any of the targets provided in Table 2 relating to cancer or any of the epitopes of Table 3. In one example, the PNME is spCas9 and the linker and antigen binding domains are inserted at ser1154.

Table 2. List of Cancer-associated Antigens that can be used for specific delivery of nucleases according to some embodiments described herein

TABLE 2
List of Cancer-associated Antigens that can be used for specific delivery
of nucleases according to some embodiments described herein

| | Example UniProt Accession ID, Chemical Name, |
|---|---|
| Target | or Literature Reference |
| cd44v6 | Tremmel et al. Blood 114: 5236-5244(2009) |
| CAIX (Carbonic Anhydrase 9, CA9) | Q16790 (CAH9_HUMAN) |
| CEA (CEA Cell Adhesion Molecule 5, | P06731 (CEAM5_HUMAN) |
| CEACAM5, Carcinoembryonic antigen) | |
| CD133 (Prominin 1, PROM1) | O43490 (PROM1_HUMAN) |
| cMet hepatocyte growth factor receptor | P08581 (MET_HUMAN) |
| (MET) | |
| EGFR (Epidermal Growth Factor | P00533 (EGFR_HUMAN) |
| Receptor, HER1) | |
| EGFR vIII | Koga et al. Neuro Oncol. 2018 Sep; 20(10): 1310- |
| | 1320. |
| EPCAM (Epithelial Cell Adhesion | P16422 (EPCAM_HUMAN) |
| Molecule) | |
| EphA2 (EPH Receptor A2) | P29317 (EPHA2_HUMAN) |
| Fetal acetylcholine receptor | Nayak et al. Proc Natl Acad Sci USA. 2013 Aug. 13; |
| | 110(33): 13654-9. |
| FRalpha folate receptor (FOLR1) | P15328 (FOLR1_HUMAN) |
| GD2 (Ganglioside G2) | (2R,4R,5S,6S)-2-[3-[(2S,3S,4R,6S)-6- |
| | [(2S,3R,4R,5S,6R)-5-[(2S,3R,4R,5R,6R)-3- |
| | acetamido-4,5-dihydroxy-6-(hydroxymethyl)oxan-2- |
| | yl]oxy-2-[(2R,3S,4R,5R,6R)-4,5-dihydroxy-2- |
| | (hydroxymethyl)-6-[(E)-3-hydroxy-2- |
| | (octadecanoylamino)octadec-4-enoxy]oxan-3- |
| | yl]oxy-3-hydroxy-6-(hydroxymethyl)oxan-4-yl]oxy-3- |
| | amino-6-carboxy-4-hydroxyoxan-2-yl]-2,3- |
| | dihydroxypropoxy]-5-amino-4-hydroxy-6-(1,2,3- |
| | trihydroxypropyl)oxane-2-carboxylic acid |
| GPC3 (Glypican 3) | P51654 (GPC3_HUMAN) |
| GUCY2C (Guanylate Cyclase 2C) | P25092 (GUC2C_HUMAN) |
| HER2 (ERBB2) | P04626 (ERBB2_HUMAN) |
| ICAM1 (Intercellular Adhesion Molecule | P05362 (ICAM1_HUMAN) |
| 1) | |
| IL13Ralpha2 (IL13RA2) | Q14627 (I13R2_HUMAN) |
| IL11 receptor alpha (IL11RA) | Q14626 (I11RA_HUMAN) |
| Kras | P01116 (RASK_HUMAN) |
| Kras G12D | P01116 (RASK_HUMAN) with G12D substitution |
| L1cam (L1 Cell Adhesion Molecule) | P32004 (L1CAM_HUMAN) |
| MAGE (melanoma-associated antigen) | P43360 (MAGA6_HUMAN) |
| | P43355 (MAGA1_HUMAN) |
| | Q9Y5V3 (MAGD1_HUMAN) |
| | P43356 (MAGA2_HUMAN) |
| | Q9UBF1 (MAGC2_HUMAN) |
| | P43364 (MAGAB_HUMAN) |
| | P43365 (MAGAC_HUMAN) |
| | Q9UNF1 (MAGD2_HUMAN) |
| | P43357 (MAGA3_HUMAN) |
| | Q9HCI5 (MAGE1_HUMAN) |
| | P43358 (MAGA4_HUMAN) |
| | P43361 (MAGA8_HUMAN) |
| | Q96JG8 (MAGD4_HUMAN) |
| | Q9HAY2 (MAGF1_HUMAN) |
| | O15481 (MAGB4_HUMAN) |
| | O15479 (MAGB2_HUMAN) |
| | P43363 (MAGAA_HUMAN) |
| | Q96M61 (MAGBI_HUMAN) |
| | P43362 (MAGA9_HUMAN) |
| | Q8TD91 (MAGC3_HUMAN) |
| | O60732 (MAGC1_HUMAN) |
| | Q9H213 (MAGH1_HUMAN) |
| | P43359 (MAGA5_HUMAN) |
| Mesothelin (MSLN) | Q13421 (MSLN_HUMAN) |
| MUC1 (Mucin 1, Cell Surface Associated) | P15941 (MUC1_HUMAN) |
| MUC16 (Mucin 16, Cell Surface | Q8WX17 (MUC16_HUMAN) |
| Associated) | |
| NKG2D (Killer Cell Lectin Like Receptor | P26718 (NKG2D_HUMAN) |
| K1, KLRK1, NK Cell receptor D, CD314) | |
| NY-ESO1 (New York Esophageal | P78358 (CTG1B_HUMAN) |
| Squamous Cell Carcinoma 1, CTAG1B, | |
| Cancer/Testis Antigen 1B) | |
| PSCA (Prostate Stem Cell Antigen, | O43653 (PSCA_HUMAN) |
| PRO232) | |
| WT1 (WT1 Transcription Factor, Wilms | P19544 (WT1_HUMAN) |
| Tumor Protein) | |
| PSMA (prostate-specific membrane | Q04609 (FOLH1_HUMAN) |
| antigen, Glutamate carboxypeptidase II, | |
| GCPII, N-acetyl-L-aspartyl-L-glutamate | |
| peptidase I, NAALADase I, NAAG | |
| peptidase, FOLH1, folate hydrolase 1) | |
| 5t4 or TPBG (Trophoblast Glycoprotein) | Q13641 (TPBG_HUMAN) |
| Transferrin receptor (TFRC, CD71, | P02786 (TFR1_HUMAN) |
| [symbol] | |
| GPNMB Breast cancer, melanoma | Q14956 (GPNMB_HUMAN) |
| (Glycoprotein Nmb) | |
| LeY (Lewis y antigen, Lewis y | N-[(3R,4R,5S,6R)-5-[(2S,3R,4S,5R,6R)-4,5- |
| Tetrasaccharide) | dihydroxy-6-(hydroxymethyl)-3-[(2R,3R,4S,5R,6R)- |
| | 3,4,5-trihydroxy-6-methyloxan-2-yl]oxyoxan-2- |
| | yl]oxy-2-hydroxy-6-(hydroxymethyl)-4- |
| | [(2R,3R,4S,5R,6R)-3,4,5-trihydroxy-6-methyloxan- |
| | 2-yl]oxyoxan-3-yl]acetamide |
| CA6 (Carbonic anhydrase 6, CA-VI) | P23280 (CAH6_HUMAN) |
| Av integrin (ITGAV, Integrin Subunit | P06756 (ITAV_HUMAN) |
| Alpha V) | |
| SLC44A4 (Solute Carrier Family 44 | Q53GD3 (CTL4_HUMAN) |
| Member 4) | |
| Nectin-4 (NECTIN4, NECT4, PVRL4, | Q96NY8 (NECT4_HUMAN) |
| EDSS1) Solid tumors | |
| AGS-16 (Ectonucleotide | O14638 (ENPP3_HUMAN) |
| Pyrophosphatase/Phosphodiesterase 3, | |
| ENPP3) | |
| Cripto (CFC1, FRL-1, Cryptic Family 1) | P0CG37 (CFC1_HUMAN) |
| ALCAM (Activated Leukocyte Cell | Q13740 (CD166_HUMAN) |
| Adhesion Molecule, CD166, MEMD) | |
| TENB2 (Transmembrane Protein With | Q9UIK5 (TEFF2_HUMAN) |
| EGF Like And Two Follistatin Like | |
| Domains 2, TMEFF2, Tomoregulin-2, | |
| HPP1, TPEF) | |
| EPCAM (Epithelial Cell Adhesion | P16422 (EPCAM_HUMAN) |
| Molecule, Tumor-Associated Calcium | |
| Signal Transducer 1, Major | |
| Gastrointestinal Tumor-Associated | |
| Protein GA733-2, Trophoblast Cell | |
| Surface Antigen 1, TACSTD1, EGP314, | |
| CD326) | |

[symbol]

indicates data missing or illegible when filed

TABLE 3
Examples of receptors with high tissue expression that may be used for tissue specific
delivery according to some embodiments of the current disclosure Gene/Protein

| | Example Gene/Protein | |
|---|---|---|
| | Symbol or Uniprot | |
| Receptor | Accession | Tissue |
| L-SIGN (CLEC4M, C-Type Lectin | Q9H2X3 (CLC4M_HUMAN) | liver |
| Domain Family 4 Member M, CD299) | | |
| ASGPR (ASGR1, ASGR2, | P07306 (ASGR1_HUMAN) | liver |
| Asialoglycoprotein receptor 1 or 2) | P07307 (ASGR2_HUMAN) | |
| AT1 (Angiotensin II Receptor Type 1, | P30556 (AGTR1_HUMAN) | kidney |
| AGTR1) | | |
| B2/B1 receptor (Bradykinin Receptor | P46663 (BKRB1_HUMAN) | lung |
| B1 or B2, BDKRB1, BDKRB2, BKRB1, | P30411 (BKRB2_HUMAN) | |
| BKRB2) | | |
| Muscarinic receptors (Muscarinic | CHRM1, CHRM2, CHRM3, | lung/Bladder |
| acetylcholine receptors, mAChRs) | CHRM4, CHRM5 | |
| FGFR4 (Fibroblast Growth Factor | P22455 (FGFR4_HUMAN) | Liver, kidney lung |
| Receptor 4) | | pancreatic cells |
| FGFR3 (Fibroblast Growth Factor | P22607 (FGFR3_HUMAN) | Brain kidney testes |
| Receptor 3) | | |
| FGFR1 (Fibroblast Growth Factor | P11362 (FGFR1_HUMAN) | Epithelial, endothelial |
| Receptor 1) | | fibroblasts |
| | | mesenchymal, |
| | | cardiomyocytes |
| Frizzled 4 (Frizzled Class Receptor 4, | Q9ULV1 (FZD4_HUMAN) | Ubiquitous |
| FZD4) | | |
| S1PR1 (Sphingosine-1-Phosphate | P21453 (S1PR1_HUMAN) | Endosomal |
| Receptor 1) | | vascular |
| | | smooth muscle |
| | | fibroblasts |
| TSHR (Thyroid Stimulating Hormone | P16473 (TSHR_HUMAN) | thyroid |
| Receptor) | | |
| GPR41 (Free Fatty Acid Receptor 3, G | O14843 (FFAR3_HUMAN) | colon |
| Protein-Coupled Receptor 41, FFAR3) | | |
| GPR43 (G Protein-Coupled Receptor | O15552 (FFAR2_HUMAN) | colon |
| 43, FFAR2, Free Fatty Acid Receptor | | |
| 2) | | |
| GPR109A (G Protein-Coupled | Q8TDS4 (HCAR2_HUMAN) | colon |
| Receptor 109A, Niacin Receptor 1, | | |
| NIACR1, Hydroxycarboxylic Acid | | |
| Receptor 2, HCAR2) | | |
| TFRC (Transferrin Receptor, CD71, | P02786 (TFR1_HUMAN) | Blood brain barrier |
| TFR1) | | |
| Insulin receptor (INSR, CD220) | P06213 (INSR_HUMAN) | Blood brain barrier |
| Insulin-like growth factor 2 receptor | P11717 (MPRI_HUMAN) | Blood brain barrier |
| (IGF2R, Cation-independent | | |
| mannose-6-prosphate receptor, CI- | | |
| MPR, MPRI) | | |
| LRP1 (LDL Receptor Related Protein | Q07954 (LRP1_HUMAN) | General cell delivery |
| 1, Apolipoprotein E Receptor, APOER, | | |
| CD91) | | |
| IGF1R (Insulin Like Growth Factor 1 | P08069 (IGF1R_HUMAN) | Prostate |
| Receptor, CD221) | | |
| Prolactin receptor (PRLR) | P16471 (PRLR_HUMAN) | Ovarian normal and |
| | | [symbol] |
| Follicle stimulating hormone receptor | P23945 (FSHR_HUMAN) | Ovarian |
| (FSHR, FSH receptor, Follitropin | | |
| Receptor, LGR1) | | |

[symbol]

indicates data missing or illegible when filed

In some embodiments, a CRISPR modified nuclease such as C9mAur or M7 ma modified with either peptide for general delivery (cationic and non specific cell entry) or grafted with a CDR peptide sequence in a loop of the 09 modified (C9m) scaffold (delivery via receptor specific binding and receptor mediated endocytosis or C9m is fused with an anti 004 nanobody either chemically or through being part of a single protein expressed in a protein expression system. A generalised transfection option is provided by complexation of any of the CRISPR enzyme derivatives described herein with a cationic peptide. In the formulations, a protein complex is provided by the formation of a complex between the monoavidin:biotin interaction with a biotinylated donor encoding a chimeric antigen receptor (sequence) and a polynucleotide modifying enzyme.

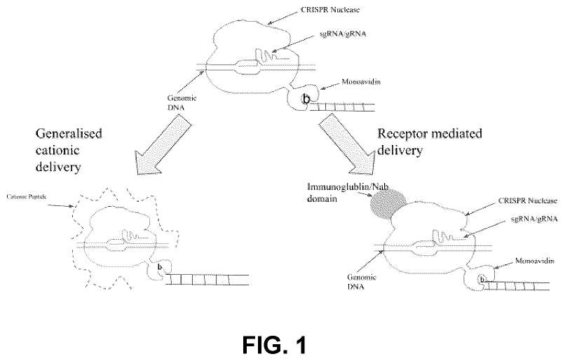

FIG. 1 shows the homology directed repair (HDR) enhanced formulations and delivery. Generalised delivery is defined herein as being driven by a cationic peptide complexation of the PNME where the nuclease is Cas9, Cas12 or Type II or Type V CRISPR system enzymes capable of producing a double strand break. Delivery will be cationic in nature non-specifically interacting with oppositely charge cell membrane. Receptor mediated delivery requires the PNME to have a domain capable of recognizing a cell receptor or marker such that it can be selectively internalized.

In some embodiments, the PNME of the present disclosure can be combined with an endosome escape domain to form a fusion polypeptide. The endosome escape domain allows the fusion peptide to exit the endosome and enter the cytoplasm after being endocytosed. The endosome escape domain can be incorporated in the sequence of the PNME or can be linked at the N or C terminus of the PNME. Table 4 details non-limitative examples of endosome escape (EE) domains.

TABLE 4
Examples of Endosome escape sequences

| SEQ ID NO: | Peptide Sequence (N- to C-terminus) |
|---|---|
| | X₁X₂X₃X₄X₅X₆X₇X₈X₉; wherein |
| | X₁ is P or C; |
| | X₂,X₃,X₄, and X₅ are independently |
| | selected from C, R, or K; |
| | and |
| | X₆,X₇,X₈, and X₉ are independently |
| | selected from C, R, K, A, |
| | or W. |
| | X₁X₂X₃X₄X₅X₆X₇X₈X₉; wherein |
| | X₁ is P or C; |
| | X₂,X₃,X₄, and X₅ are independently |
| | selected from C, R, or K; |
| | and |
| | X₆,X₇,X₈, and X₉ are independently |
| | selected from C, R, K, A, |
| | or W., and wherein at least 3 of |
| | X₁-X₉ are C and no more |
| | than 8 of X₁-X₉ are C. |
| 13 | PCRKCACCA |
| 14 | PRCCRWCCA |
| 15 | PRRCKRCKC |
| 16 | CKKCRKCCK |
| 17 | CCRCKCWCC |
| 18 | CCRKCCCCC |
| 19 | PRKCCCCCC |
| 20 | HHHHHHHHHH |
| 21 | CCCCCC |

Double strand breaks caused by CRISPR can be repaired either by non-homologous end joining (NHEJ) or through homology directed repair (HDR) or single strand annealing. In the case of CRISPR editing NHEJ is preferred as for the majority of the cell cycle it is the predominant method of resolving double strand breaks by the action of Ku70/80, artemis, DNA-Pk and lig4, resulting in small insertion and deletions through can inactivate a gene. Where a donor template is present and cell cycle permits (S1 G2) homologous recombination can guide repair where the donor provides a template for the double strand break to be resolved. Unfortunately HDR with CRISPR is highly inefficient due to many factors, among which is the availability of donor DNA at the point of double strand break formation, and the limited period of the cell cycle when HDR is preferred. To resolve the donor availability, donor DNA can be associated via the biotin interaction to the fusion CRISPR proteins where a nuclease is expressed with a Monoavidin domained attached. This is an advantage over other gene delivery systems such as viral delivery (e.g. AAV) due to packaging volumes constraints. Indeed, CRISPR nucleases and donors have to be delivered separately and the advantage of co-localisation is lost both in time domain and spatial co-localisation. Other iterations of co-localisation have used snap tag, aptamers and nanoparticle systems. The advantages of the present system are the use of a protein with additional endosomal escape function and optional delivery via generalised cationic methods or preferentially receptor mediated delivery.

In some embodiments, the PNME further comprises a hapten binding domain to link an additional protein or nucleic acid ligand to the PNME. A “hapten binding domain” is a peptide or oligonucleotide domain that binds a hapten. “Hapten” refers to a small molecule, which when combined with a larger carrier such as a protein, is capable of high affinity binding to an antibody or antibody mimetic (“hapten binding domain”). In some embodiments, hapten/hapten binding domain pairs are derived from natural proteins or engineered variants thereof, such as the biotin/avidin pair or amylose/MBP pair. Engineered alternatives for biotin include D-desthiobiotin. Alternatives for avidin include streptavidin, NeutrAvidin, and CaptAvidin. In some embodiments, hapten/hapten binding domain pairs are synthetically engineered pairs such as 3-methylindole/anti-3-methylindole monoclonal antibody (such as 14G8, 3F12, 4A1G, 8F2, or 8H1 monoclonal antibodies), fumonisin B1/anti-fumonisin antibody, 1,2-Naphthoquinone/anti-1,2-Naphthoquinone antibody, 15-Acetyldeoxynivalenol/anti-15-Acetyldeoxynivalenol antibody, (2-(2,4-dichlorophenyl)-3(1H-1,2,4-triazol-1-yl)propanol)/anti-(2-(2,4-dichlorophenyl)-3(1H-1,2,4-triazol-1-yl)propanol) antibody, 22-oxacalcitriol/anti-22-oxacalcitriol antibody, (24,25(OH)2D3)/anti-(24,25(OH)2D3) antibody, 2,4,5-Trichlorophenoxyacetic acid/anti-2,4,5-Trichlorophenoxyacetic acid antibody, 2,4,6-Trichlorophenol/anti-2,4,6-Trichlorophenol antibody, 2,4,6-Trinitrotoluene/anti-2,4,6-Trinitrotoluene antibody, 2,4-Dichlorophenoxyacetic acid/anti-2,4-Dichlorophenoxyacetic acid antibody, 2-hydroxybiphenyl/anti-2-hydroxybiphenyl antibody, 3,5,6-trichloro-2-pyridinol/anti-3,5,6-trichloro-2-pyridinol antibody, 3-Acetyldeoxynivalenol/anti-3-Acetyldeoxynivalenol antibody, 3-phenoxybenzoic acid/anti-3-phenoxybenzoic acid antibody, digoxin/anti-digoxin antibody, fluorescein/anti-fluorescein antibody, or hexahistidine/Ni-NTA. The hapten binding domain can be located N- or C-terminal to the PNME, or both. The hapten binding domain can be separated from another domain described herein by a linker or can be directly fused to the domain sequence without intervening amino acids. In some cases, the hapten binding domain is within a linker domain separating two other domains of the PNME. In some cases, the PNME comprises at least one, at least two, at least 3, at least 4, at least 5, or more hapten binding domains.

In some embodiments there is provided a composition comprising the PNME and a hapten-binding domain. The composition can further comprise a peptide, protein, oligonucleotide, or polynucleotide linked to the corresponding hapten. The oligonucleotide can comprise a deoxyribonucleotide or a ribonucleotide. The oligonucleotide can comprise a single-stranded or double-stranded oligonucleotide.

In some embodiments when the PNME comprises a hapten-binding domain and a programmable or site directed nuclease, the PNME further comprises a nucleic acid with homology arms complementary to regions flanking the target site for the programmable or site directed nuclease (e.g. a repair template or donor DNA). By this method, a nuclease can be delivered to the cell in vicinity of the site to be cleaved. In some cases, the repair template or donor DNA is a single- or double-stranded DNA repair template or donor DNA comprising from 5′ to 3′: a first homology arm comprising a sequence of at least about 20 nucleotides 5′ to the target sequence, an insert DNA sequence or region of at least about 10 nucleotides, and a second homology arm comprising a sequence of at least about 20 nucleotides 3′ to the target sequence. In some embodiments, the first or said second homology arms comprise a sequence of at least about 20, 40, 50, 80, 120, 150, 200, 300, 500, or 1000 nucleotides. In some embodiments, the 5′ and 3′ homology regions have different lengths. In some embodiments, the 5′ and 3′ homology regions have the same length. In some embodiments, the repairtemplate or donor DNA is a single stranded polynucleotide and the 5′ homology region comprises 50-100 nucleotides and the 3′ homology region comprises 20-60 nucleotides. In some embodiments, the 3′ end of the 5′ homology region is homologous to a sequence within 5 nucleotides of the double-stranded break. In some embodiments, the 5′ end of the 3′ homology region is homologous to a sequence within 5 nucleotides of the double strand break. The insert region can comprise an exon, an intron, a transgene, a stop codon (e.g. a stop codon in frame with the gene ORF into which it is inserted), a coding sequence of a gene comprising at least one nonsense or missense mutation, or a mutation ablating activity of a PAM site in the vicinity of a sequence targeted by a PNME CRISPR enzyme. Example transgenes include selectable markers such as BlaS, HSV-tk, puromycin N-acetyl-transferase, or Tn5 NEO gene, which can be used to select for cells that have undergone recombination with the donor DNA or repair template. Example transgenes also include detectable labels such as fluorescent enzymes, proteins sequences capable of high-affinity detection with antibodies, epitope tags, or fluorescent proteins.

In one example, the PNME is built on a C9m scaffold, where a fusion of Cas9 is made with a mono avidin domain, with peptide sequences or an antigen binding domain grafted on to loop domains identified above. The antigen binding domain (e.g. VHH) can be selected as binders targeting specific receptors, for example CD4, CD8, CD16 or CD56. Grafting of the antigen binding domain can be achieved by insertion of the corresponding DNA sequence to an expression vectors encoding the C9m.

In some embodiments, the PNME can comprise a nuclear localization sequence (NLS). The NLS can be located at the N- or C-terminus of the PNME, or both. The NLS can be separated from the PNME peptide sequence by a linker or can be directly fused to the PNME sequence without intervening amino acids. In embodiments, the PNME comprises at least one, at least two, at least 3, at least 4, at least 5, or more NLSs. In some embodiments, NLSs comprise 7-25 amino acid residues. In some embodiments, NLSs are derived from mammalian nuclear entering proteins such as splicing factors or transcription factors. In some embodiments, an NLS interacts with an importin. In some embodiments, the NLS is a bipartite NLS wherein amino acids within an N-terminal portion of the NLS involved in the recognition of an importin and amino acids within a C-terminal portion of the NLS involved in the recognition of an importin are split by an amino acid sequence not involved in the recognition of an importin. In some embodiments, an NLS comprises at least one sequence depicted in Table 5 below or a combination of sequences from Table 5 (i.e. SEQ ID NOs: 22-37), a sequence having at least 70%, at least 75%, at least 80%, at least 85%, at least 90%, at least 95%, at least 99% sequence identity to a sequence described in Table 5, or a sequence identical to any of the sequences in Table 5. When more than one NLS is included in a PNME or PNME composition, the NLSs may comprise the same sequence or comprise different sequences. In some embodiments, two or more NLS sequences are included (e.g. NLS of SV40) and the NLS sequences can be positioned in a linker between the PNME and a mono avidin domain.

TABLE 5
Examples of Nuclear Localization
Sequences (NLSs)

| SEQ ID | |
|---|---|
| NO: | Peptide Sequence (N- to C-terminus) |
| 22 | KRRRRQERAKEREKRR |
| 23 | MRKTKALAPTA |
| 24 | KKKRRP |
| 25 | KKFK |
| 26 | KKKKYN |
| 27 | PPAKRERLD |
| 28 | RGRGRRRRRRRR |
| 29 | PKKNKLKKKS |
| 30 | PKKKRKV |
| 31 | NYKRPMDGTYGPPAKRHEGE |
| 32 | KRSGSKAF |
| 33 | PPAKRERLD |
| 34 | RKKSGMQIALNDHLKQRR |
| 35 | KKAFQNVLRIQCLCRK |
| 36 | RRLLCRCGRRLPPEPCAAARPALFPSGVPAARSSP |
| 37 | SVLGKRKFA |

In some embodiments, the PNME is bi-specific, that is to say carrying two domains or peptide sequences that can recognise the same or different cell receptors (e.g. T cell receptors). The cell recognition domain, the display domain and/or the antigen binding domain can be combined to form bi or multi specific protein complexes. Accordingly a bispecificity can be produced where the PNME effectively has two receptor binding domains (e.g. a display domain such as a CDR and a cell recognition domain, or a display domain such as a CDR and an antigen binding domain such as an inserted VHH).

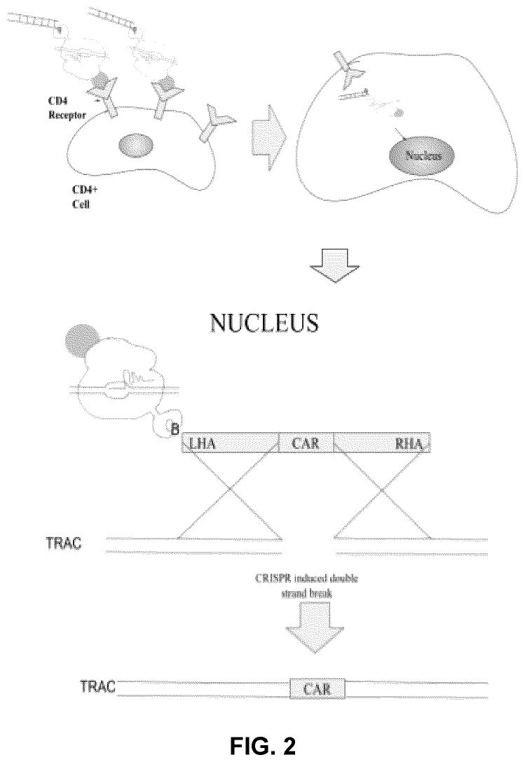

In some embodiments, there is provided a CRISPR system for introduction of a CAR by first formulating a CRISPR protein complex via either of the delivery mechanisms (cationic or receptor mediated) with a sgRNA molecule specific to TRAC. The intention is to introduce a chimeric antigen receptor by forming double strand break in the early exons of the TRAC receptor, removing its native expression and placing the CAR under the control of the endogenous promoter of the TRAC gene. FIG. 2 illustrates the system mechanism of action with respect to receptor mediated delivery of the protein complex targeting CD4 receptor for delivery. The anti CD4 domain binds a cell receptor and get internalized by receptor mediated endocytosis. Then, endosomal escape is affected by the endosome escape domain (e.g. endosomal peptide escape sequences) and a transit to the nucleus is achieved by the NLS. In the nucleus, a homologous recombination is performed with the genomic DNA and repair template homologous sequences (left and right homology arms) flanking CAR insert on donor DNA.

To achieve co delivery once the protein complex has been formed (e.g. CRISPR nuclease plus guide RNA) the biotin mono avidin relationship is exploited by biotinylating a CAR encoded donor DNA molecule with a 5′ or 3 or internal biotin label. Mixing the CRISPR nuclease in equal molar parts enables binding of the donor to the protein complex. At this point the enhanced HDR CRISPR complex is ready for delivery to cells. If using a CRISPR protein complex without anti CD4 binding domain and a cationic peptide is used this will enable delivery non specifically via the cationic interaction of the peptide with the oppositely charged cell membrane. Where an anti CD4 domain is present, delivery will be achieved to cells by interaction with the CD4 receptor upon a T-cell or T-cell model.

Compared to virus vectors, the presently described genetic delivery systems have low immunogenicity, increased biosafety, decreased production costs, and a capacity to transduce large gene fragment>100 kb in length, making them an advantageous means for CAR insertion into T cell genomes, including NK cells, with long-lasting expression

In some embodiments, there is provided a vector comprising a nucleotide sequence encoding a PNME. In some cases, the vector further comprises a hapten-binding domain within the same open reading frame (ORF) as the endosome escape domain, and PNME. A “vector” is a nucleic acid sequence capable of transferring other operably-linked heterologous or recombinant nucleic acid sequences to target cells. In some examples, a vector is a minicircle, plasmid, yeast artificial chromosome (YAC), bacterial artificial chromosome (BAC), cosmid, phagemid, bacteriophage genome, or baculovirus genome. Suitable vectors also include vectors derived from bacteriophages or plant, invertebrate, or animal (including human) viruses such as CELiD vectors, adeno-associated viral vectors (e.g. AAV1, AAV2, AAV4, AAV5, AAV6, AAV7, AAV8, AAV9, or pseudotyped combinations thereof such as AAV2/5, AAV2/2, AAV-DJ, or AAV-DJ8), retroviral vectors (e.g. MLV or self-inactivating or SIN versions thereof, or pseudotyped versions thereof), herpesviral (e.g. HSV- or EBV-based), lentiviral vectors (e.g. HIV-, FIV-, or EIAV-based, or pseudotyped versions thereof), adenoviral vectors (e.g. Ad5-based, including replication-deficient, replication-competent, or helper-dependent versions thereof) or baculoviral vectors (which are suitable to transfect insect cells as described herein). In some embodiments, a vector is a replication competent viral-derived vector.

Accordingly, in some aspects the present disclosure also provides for host cells comprising any of the vectors described herein. In some embodiments, the host cells are animal cells. The term “animal cells” encompasses any animal cell, including but not limiting to, invertebrate, non-mammalian vertebrate (e.g., avian, reptile, and amphibian), and mammalian cells. A number of mammalian cell lines are suitable host cells for recombinant expression of polypeptides of interest. Mammalian host cell lines include, for example, COS, PER.C6, TM4, VERO076, MDCK, BRL-3A, W138, Hep G2, MMT, MRC 5, FS4, CHO, 293T, A431, 3T3, CV-1, C3H10T1/2, Colo205, 293, HeLa, L cells, BHK, HL-60, FRhL-2, U937, HaK, Jurkat cells, Rat2, BaF3, 32D, FDCP-1, PC12, M1x, murine myelomas (e.g., SP2/0 and NSO) and C2C12 cells, as well as transformed primate cell lines, hybridomas, normal diploid cells, and cell strains derived from in vitro culture of primary tissue and primary explants. Any eukaryotic cell that is capable of expressing recombinant and/or transgenic proteins may be used in the disclosed cell culture methods. Numerous cell lines are available from commercial sources such as the American Type Culture Collection (ATCC). The host cells can be CHO cells. In some embodiments, the host cells are bacterial cells suitable for protein expression such as derivatives of E. coli K12 strain. In some embodiments, the host cells comprise plant cells into which genes have been introduced by a vector single-stranded RNA virus tobacco mosaic virus. “Host cells” can be insect cells which are utilized for the production of large quantities of the polypeptides according to the disclosure. In some embodiments, the baculovirus system (which provides all the advantages of higher eukaryotic organisms) is utilized. The host cells for the baculovirus system include, but are not limited to Spodoptera frugiperda ovarian cell lines SF9 and SF21 and the Trichoplusia ni egg-derived cell line High Five.

In some embodiments, the PNME described herein is delivered to cells (e.g. in vitro or in vivo) via a pharmaceutical composition or dose form of particular design. The pharmaceutical composition may comprise sterile water alongside a pharmaceutically acceptable excipient, and optional electrolytes to ensure the composition is isotonic. Because the PNME and the pharmaceutical composition comprising same as described herein do not require chemical transfection agents to enter cells, in some embodiments, a liquid formulation for delivery does not comprise a polyetherimide (PEI), polyethylene glycol (PEG), polyamidoamine (PAMAM), or sugar (dextran) derivative polymer comprising more than three subunits.

The CAR-T cells of the present disclosure can be engineered to target diverse antigens, enhance the proliferation and persistence in vivo, increase infiltration into solid tumours, overcome resistant tumour microenvironment, and ultimately achieve an effective anti-tumour response.

In some embodiments, the T cell target is a CD8 T cell. The cytotoxic CD8 T cells can eliminate tumor cells through recognition of peptide epitopes presented on major histocompatibility complex class I (MHC-I) molecules by the alpha-beta T cell receptor (apTCR). T cells can recognize peptides derived from tumor-associated antigens, cancer-testis antigens, viral antigens (in the case of virally derived tumors), and neoantigens. Neoantigens are peptides derived from mutated “self” proteins that the immune system detects as “nonself.” Many neoantigens are “private” to individual tumors, and immunity to these antigens can be exploited with immunotherapies such as checkpoint blockade or personalized vaccines. Some neoantigens are derived from common or “hotspot” mutations such as those arising in the RAS proteins and p53. The RAS family (H, N, and KRAS) of small GTPases are among the most commonly mutated oncogenes in cancer. Among them, the G12D mutation in KRAS occurs most frequently.

In some embodiments, a safety check is introduced in the CAR T cells. This can be done by the inclusion of a suicide gene to knock back CAR cells if a cytokine storm situation occurs. This achieved by packaging the gene in place of the GFP on the CAR, and then minimizing donor size in base pairs. In such embodiments, interchangable CAR heads can be obtained by placing a monoavidin domain in place of the scFV, enabling a generic CAR T cell to be created, and easy exchange of targeting. An anti-CD4 targeting DNA complex can carry and introduce a donor DNA, with a standard CAR design but with the scFV exchange for monoavidin (MAV). After generation of the CAR-CD4+ T cells, activation of the cells towards a specific cell marker can be achieved by intravenously injecting a biotinylated scFV, nanobody, circular peptide, or antibody mimetic, which will then bind to the CAR-CD4+ T cell. As long as the biotinylated ligand is in excess and expansion occurs due to the interaction of the MAV:biotinylated ligand with target cell, as expansion occurs the new cells will lack the biotinylated target. These cells become in a situation where the biointylated ligands is in excess and systemically distributed which will immediately associate with ligand and target the appropriate cells. A conceptual advantage of such embodiments is that as selective pressure results in selection of cancer cells lacking the original target receptor (e.g. CD19) a subsequent maintained receptor could be immediately selected. For example, CD19 targeting can be switched to CD22 targeting by intravenuus injection of a CD22 biotinylated VHH, which can switch the targeting without further genetic manipulation.

An autologous CAR-T cell therapy can comprise several steps. First, T cells are isolated from a patient's or donor's blood. Subsequently, cells are transduced with CAR-encoding genes using the protein complexes described herein. CAR-modified immune cells are expanded until sufficient cell numbers are attained and are adoptively transferred into the patient to fight malignant cells. Prior to infusion of the CAR-modified immune cells, lymphodepletion is performed in most therapeutic settings to allow efficient cell engraftment.

Conventional treatment of cancer includes radiotherapy, chemotherapy, and surgery. These are associated with poor efficacy and significant side effects. Therefore, novel strategies with higher efficacy and fewer complications, such as CAR-T based immunotherapy, have been developed. Immunotherapy is the modification and enhancement of the host immune system to combat different pathologies, such as cancer. Adoptive cell therapy (ACT) is a type of immunotherapy that includes the application of immune cells to treat cancer of which CAR T cell therapy is an example.

In some embodiments, the CAR-T cell therapy can be combined with other therapies such as chemotherapy, radiation therapy, and immune checkpoint blockade. The CAR-T cell therapy may allow for a reduce dose of chemotherapy or radiation therapy when used in combination which would reduce the side effects suffered by the patient receiving these traditional treatments.

The evolution of resistance in cancer populations is a major factor limiting patient remission and curing. One way to mitigate the development of resistance is to make a bi-specific CAR T cell, this way if one receptor is selected against, the other can replace it and be relied on for the target cell binding. The situation where the two receptors of the bi-specific CAR T cell are selected against and the cancer evolves to prevent or their lower expression, has a much lower probability than a single receptor mutational change being selected for in the case of a monospecific CAR T cell. One alternative approach as explained above is the MAV-CAR where, interchangeability is achieved upon a “headless or exchanging platform”, by the addition of a biotinylated receptor binder. Yet another approach to tackle cancer resistance is to evaluate a patient for the change of cancer cell expression (for example by flow cytometry) and then inject the protein complex of the present disclosure with a donor that targets an alternative receptor validated to be expressed upon the cancer cells. This approach could also benefit from the cancer patient being used to pre-select the appropriate binder from a library of VHH, antibodies mimitics or peptides to provide a robust validation prior to treatment of the cellular recognition.

Examples of diseases that can be treated by the present CAR T cell therapies and some exemplary cell targets for each disease are provided: multiple myeloma (MM) (CD138, CS1), glioblastoma (EGFR, EGFRVIII, CD73, HER2), lymphoma (CD22, CD19, CD4), acute lymphocytic leukemia (ALL) (CD7, CD19, CD5, FLT3), acute myelocytic leukemia (AML) (CD33, CD19, CD4, CD123), chronic lymphocytic leukemia (CLL) (CD19), breast cancer (HER2, EpCAM, TF, EGFR), colorectal cancer (HER2, EpCAM, NKG2D, MUC 1), ovarian cancer (HER2, mesothelin), renal cell carcinoma (RCC) (HER2, EGFR), prostate (PSMA), neuroblastoma (GD2, CD244, CD276), melanoma (GPA7), Ewing sarcoma (GD2), Hepatocellular cancer (HCC) (GPC3), pancreatic cancer (MUC 1), gastric cancer (MUC 1), non-small cell lung cancer (MUC 1), hepatocellular carcinoma (MUC 1), glioma (MUC 1), triple-negative breast cancer (TNBC) (MUC 1), and B cell malignancies (CD19, CD20).

In some embodiments, allogeneic CAR NK cells are produced using the delivery and genetic editing described herein. Allogeneic CAR NK cells generally have reduced risk for graft versus host disease (GVHD). Moreover, cytokine release syndrome (CRS) and neurotoxicity are less likely to occur in CAR-NK immunotherapy partly due to a different spectrum of the secreted cytokines: activated NK cells usually produce IFN-γ and GM-CSF, whereas CAR-T cells predominantly induce cytokines, such as IL-1a, IL-1Ra, IL-2, IL-2Ra, IL-6, TNF-α, MCP-1, IL-8, IL-10, and IL-15, that are highly associated with CRS and severe neurotoxicity.

In some embodiments, subsequent genetic modification can be performed after the introduction of the CAR construct by the protein complex of the present disclosure. For example, the genetic ablation of PD1 can improve T cell function and in the case of cancer treatment also improves tumour targeting and treatment efficacy. In embodiments where NK cells are the CAR T cells, B2M can be genetically ablated or other genetic modifications that interfere with the HLA presentation.

Allograft rejection is mainly driven by CD8 T-cell, CD4 T cells, NK cells and, to a lesser extent, by macrophages. However, in the context of CAR T-cell therapy, the relative contribution of these cell types to allograft rejection may vary depending on their absolute numbers and reconstitution kinetics following preconditioning regimen. In some embodiments, a dual targeting approach and adapter CARs is used in order to avoid therapy resistance caused by antigen loss.

### Example

### Materials

The following reagents were purchased from Wisent™: Dulbecco's Modified Eagle Medium (DMEM), fetal bovine serum (FBS) premium heat deactivated, Penicillin/streptomycin (Pen/Strep), F12, Luria Bertani (LB), peptone, yeast extract and super broth. The following reagents were purchased by Biobasic: ethanol, isopropanol, phosphate buffer saline (PBS), DNA ladder 1 kb, DNA ladder 100 bp, Protein Ladder 250 kda, 33:1 acrylamide pre mix, N-2-hydroxyethylpiperazine-N-2-ethane sulfonic acid (HEPES), tris(hydroxymethyl)aminomethane (TRIS), glucose, arabinose, NaCl, KCl, HCl, Ammonium Hydroxiude, Calcium chloride, SOC broth, ethylenediaminetetraacetic acid (EDTA), agar, agarose, Tris-acetate-EDTA (TAE) 50× buffer, micropipette tips, serological pipettes (10 ml, 25 ml, 5 ml), 15 ml sterile tubes, 1.5 ml sterile tubes 50 ml sterile tubes, PCR tubes, Culture plates (6 well, 12 well, 24 well, 96 well flat, 96 well round bottom, 10 cm plates), and Plastic Petri Dishes. The following Monarch RNA Cleanup Columns was purchased from BioLabs™ which includes Monarch DNA, RNA, Plasmid prep kits and restriction enzymes, T7 endonuclease I (and buffer NEB 2.0), Protease K, hifi assembly mix, and PCR enzymes. PCR enzymes were obtained from Transgen™ and the primers from Biocorp™. Mutagenesis service were provided by ABM™. Primers and gblocks were obtained from IDT™. Large DNA synthesis was performed with TwistBio™. The following reagents were purchased from Thermofischer™: pierce dye removal columns, 4 ml bacterial culture tubes, PCR enzymes (Direct Phire/Phusion), Various fluorescent dyes (DAPI, NHS fluorescence), Luminoprobe: Cy5.5 NHS ester and TAMRA nhs ester. NiNTA beads and the endotox kit were purchased from Genscript™.

### Chimeric Antigen Receptor DNA Donor Construct

Donor DNA for the CAR antigen receptor was encoded in the following manner to contain domains required for CAR function:
Left homology arm (LHA): a sequence homologous to the exon1 TRAC loci located around the guide positions that dictate the position of the double strand break,
CD28 signal peptide: translocation to cell membrane,
Three Flag tags: for the identification of CAR construct,
Anti CD19 scFV: for recognition of CD19 upon B cell lymphoma cells,
CD8 hinge region: couples anti CD19 to transmembrane domain and allows transmission of signal through CAR protein construct, presentation of the anti CD19 domain and receptor expression is also influenced by the CD8 hinge region sequence and amino acid length,
CD28 transmembrane domain: allows presentation of receptor in bi-lipid membrane,
Cd3z: intracellular signalling and initiation of T-cell anti-cancer function,
P2A: cleavage domain that removes down stream peptide sequence from rest of CAR construct, releasing in this case a eGFP fluorescent protein tag,
eGFP: green fluorescent protein tag to confirm both in-frame CAR insertion to genome and receptor full length expression, presence of P2A cleavage sequence prevent eGFP being part of the final CAR receptor
Posttranscriptional Regulatory Element (WPRE) sequence to improve RNA stability and protein yield, in frame with GFP, and
Right homology arm (RHA): a sequence homologous to the exon1 TRAC loci located around the guide positions that will dictate the position of double strand break.

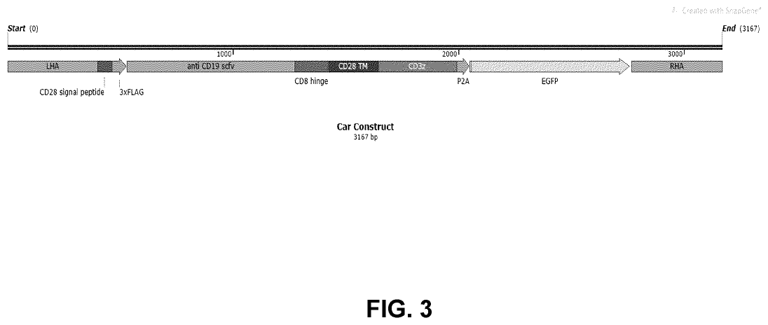

The map of the CAR construct is shown in FIG. 3.

### Vectorisation

Bacterial expression vectors using T7 promoters were used to expressed proteins in E. coli. Inserts were synthesized to encode the PNME and complementary sequences. Vectors apply a pB322 origin, repressor of primer (ROP) element for low copy number, kanamycin or amplicillin resistance genes, Lac Repressor for inhibition of transcription until isopropyl β-D-1-thiogalactopyranoside (IPTG) is introduced, T7 promoter and ribosomal binding site, completes the basic architecture of the expression vectors. Vector plus inserts were ordered through commercial suppliers or produced from a library of DNA parts for each component and assembly using either golden gate or Gibson assembly. The base C9m and M7 bacterial expression vectors were synthesized by assembly cloning.

For grafting inserts of under 15 amino acids or 30 DNA base pairs to vector sequences, site directed mutagenesis services from commercial suppliers were utilised, where the base C9m and M7 nuclease expression vectors were the template vectors and inserts were determined by the desired amino acid sequence required at the insertion site labeled “SP3” as required. The SP3 site is on an external loop of spCas9 and was identified as a suitable location for the insertion of an antigen binding domain. More specifically, the site SP3 is ser1154 which is situated as part of a loop domain that is external and not obscured. The designs decided upon included “Zero”-no linker, “L1”—N terminal linker to improve VHH presentation, and “L2”—alternative linker sequence of 23 amino acids to improve VHH presentation and offer greater flexibility of VHH presentation.

TABLE 6
Primer list

| Name | Sequence | SEQ ID NO |
|---|---|---|
| C9mRpnew | CGATTTCCCTTTTTCCACCTTAGCAA | 38 |
| | CCACTAGGAC | |
| C9mFpnew | CACCGTTACCGTTAGCAGCAAGAAGT | 39 |
| | TAAAATCCGTTAAAG | |
| C9m_fwd | AAGAAGTTAAAATCCGTTAAAG | 40 |
| C9m_rev | CGATTTCCCTTTTTCCAC | 41 |
| minC4n_ | AGGTGGAAAAAGGGAAATCGGATCGA | 42 |
| fwd | TGGGGATCCCAG | |
| Common | TTAACGGATTTTAACTTCTTGCTGCT | 43 |
| C4n_rev | AACGGTAACGGTG | |
| L1 For | GGGAGCGCAGGATCCGCTGCCGGTTCA | 44 |
| | GGAGAGTTTGATCGATGGGGATCCCAG | |
| L2 For | AAAGAGTCGGGAAGCGTCTCTTCGGAA | 45 |
| | CAGTTAGCGCAGTTCCGCTCACTGGAT | |
| | GATCGATGGGGATCCCAG | |
| L1_c4n2c9m | AGGTGGAAAAAGGGAAATCGGGGAGCG | 46 |
| fwd | CAGGATCCGCT | |
| L2_C4n2c9m | AGGTGGAAAAAGGGAAATCGAAAGAG | 47 |
| fwd | TCGGGAAGCGTC | |

### Creating the Common Backbone for Insertion of all Fragments

The common C9mAur fragment was amplified using the following primers: C9m_fwd and C9m_rev. The template for amplification was C9mAur vector. The product size was confirmed by gel electrophoresis. The fragment linearized the plasmid and split C9mAur at the location of the loop domain we are going to clone into. A Dpn1 treatment was performed to digest the template plasmid. Dpn1 was first deactivated (PCR cleanup column of fragment and quantification), and the Dpn1 digestion was confirmed by DH5a transformation resulting in zero colonies.

### Creating “Zero” Linker Insert: C4n

The C4n1 amplification fragment was performed with the primers minC4n_fwd and commonC4n_rev and the template for the amplification was C4n Vector. The product size was confirmed by gel electrophoresis. A Dpn1 treatment was performed to digest the template plasmid. Dpn1 was first deactivated (PCR cleanup column of fragment and quantification), and the Dpn1 digestion was confirmed by DH5a transformation resulting in zero colonies. The resulting fragment was purified and quantified and was then used for cloning.

### Adding Linkers (L) to C4n

Two linkers (L1 and L2) were added to C4n the “Zero” since it contained zero linkers. The primers L1 For and L2 For from Table 7 were used to add the 5′ linkers to C4n, resulting in L1-C4n and L2-C4n (i.e. the template for amplification was the C4n vector). The amplification employed a common reverse primer common C4n_rev. The amplification created the reverse overhang using the common primer and introduced the linker at the 5′ end of the C4n VHH sequence. A PCR clean up purification of both L1-C4n and L2-C4n was then performed.

TABLE 7
Linkers L1 and L2

| Name | Sequence | SEQ ID NO |
|---|---|---|
| L1 (DNA) | GGGAGCGCAGGATCCGCTGCCGGTTC | 48 |
| | AGGAGAGTTTGATCGATGGGGATCC | |
| L1 (a.a) | GSAGSAAGSGEFDRWGS | 49 |
| L2 (DNA) | AAAGAGTCGGGAAGCGTCTCTTCGGA | 50 |
| | ACAGTTAGCGCAGTTCCGCTCACTGG | |
| | ATGATCGATGGGGATCC | |
| L2 (a.a.) | KESGSVSSEQLAQFRSLDDRWGS | 51 |

Introduction of Overhangs for Gibson Cloning with C9mAur Fragment

The fragments with overhang primers were amplified to introduce overhangs to a C9m fragment. The amplification of L1-C4n was performed with the L1_c4n2c9m_fwd forward primer and the amplification of L2-C4n was performed with the L2_C4n2c9m_fwd forward primer. The amplification used the common reverse primer C4n_Comm_rev. The product size was confirmed by gel electrophoresis. A Dpn1 treatment was performed to digest the template plasmid. Dpn1 was first deactivated (PCR cleanup column of fragment and quantification), and the Dpn1 digestion was confirmed by DH5a transformation resulting in zero colonies. The resulting fragment was purified and quantified and was then used for cloning.

### Vectors Obtained

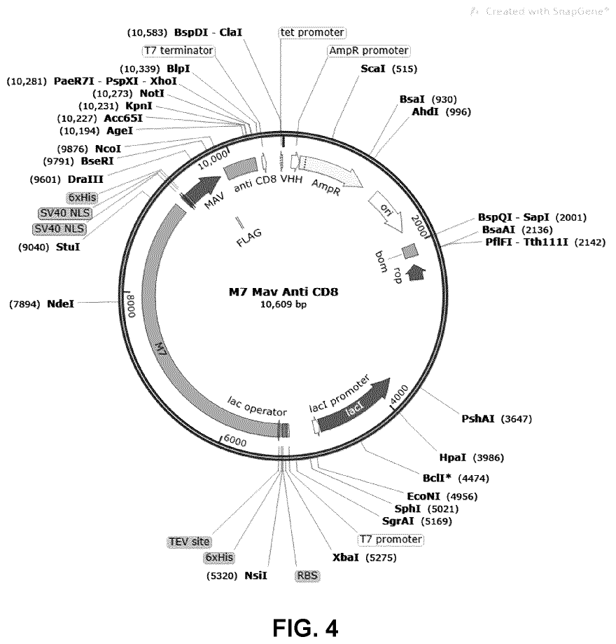

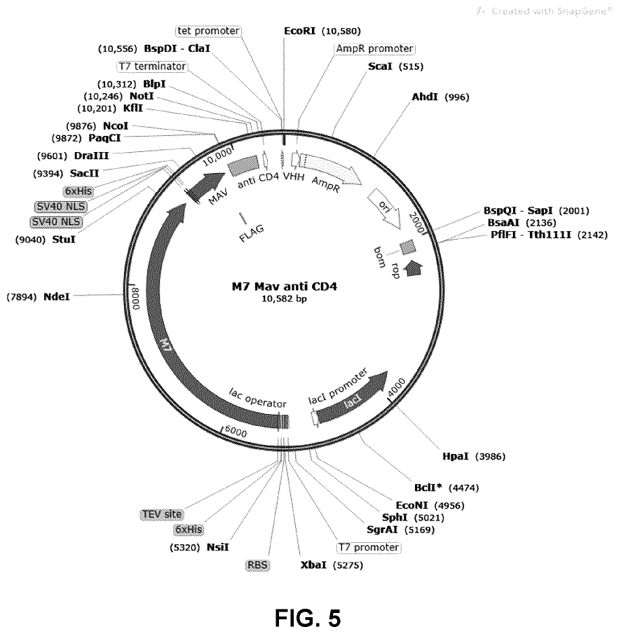

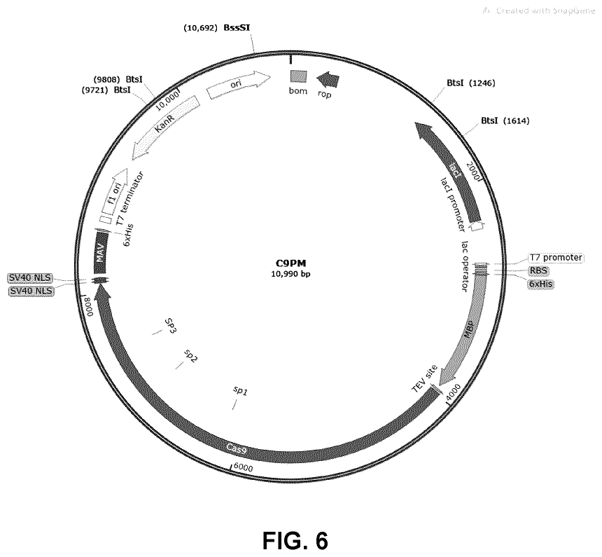

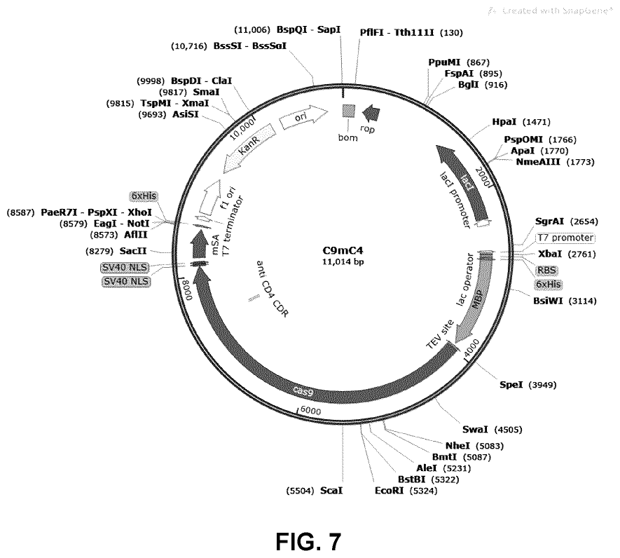

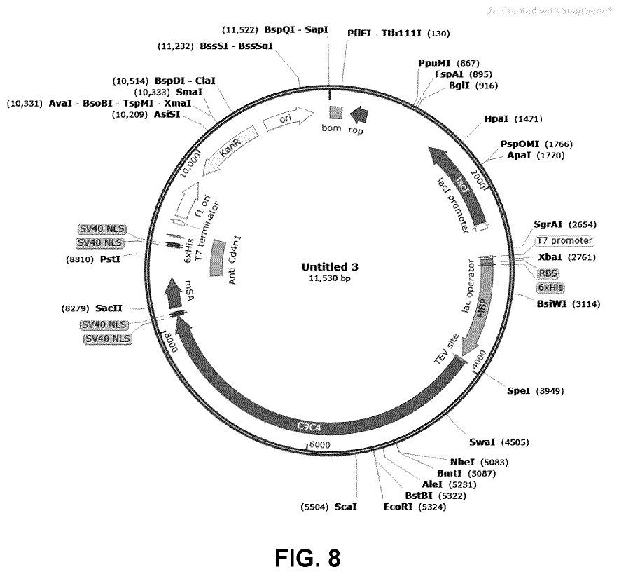

The vectors are synthesized are shown in FIGS. 4-8. More specifically, FIG. 4 shows the map of the vector of M7 Mav anti CD8. FIG. 5 shows the map of the vector of M7-Mav-anti CD4. FIG. 6 shows the map of the vector of C9mAur. FIG. 7 shows the map of the vector of C9mC4. FIG. 8 shows the map of the vector of C9C4 anti CD4n1.

TABLE 8
Vector sequences

| Name | Sequence | SEQ ID NO |
|---|---|---|
| Basic CAR | AGTTTGCTTTGCTGGGCCTTTTTCCCATGCCTGCCTTTACTCTGCCAGAGTT | 52 |
| construct | ATATTGCTGGGGTTTTGAAGAAGATCCTATTAAATAAAAGAATAAGCAGTAT | |
| | TATTAAGTAGCCCTGCATTTCAGGTTTCCTTGAGTGGCAGGCCAGGCCTGGC | |
| | CGTGAACGTTCACTGAAATCATGGCCTCTTGGCCAAGATTGATAGCTTGTGC | |
| | CTGTCCCTGAGTCCCAGTCCATCACGAGCAGCTGGTTTCTAAGATGCTATTT | |
| | CCCGTATAAAGCATGAGACCGTGACTTGCCAGCCCCACAGAGCCCCGCCCTT | |
| | GTCCATCACTGGCATCTGGACTCCAGCCTGGGTTGGGGCAAAGAGGGAAATG | |
| | AGATCATGTCCTAACCCTGATCCTCTTGTCCCACAGATGCTCAGGCTGCTCT | |
| | TGGCTCTCAACTTATTCCCTTCAATTCAAGTAACAGGAGGGTCTTCGGACTA | |
| | CAAGGATCATGACGGAGACTATAAGGATCACGATATTGATTACAAAGATGAC | |
| | GACGACAAAGACATCCAGATGACACAGACTACATCCTCCCTGTCTGCCTCTC | |
| | TGGGAGACAGAGTCACCATCAGTTGCAGGGCAAGTCAGGACATCTCTAAGTA | |
| | TTTGAATTGGTATCAGCAGAAACCAGATGGAACTGTTAAACTCCTGATCTAC | |
| | CATACATCAAGATTACACTCAGGAGTCCCATCAAGGTTCAGTGGCAGTGGGT | |
| | CTGGAACAGATTATTCTCTCACCATTAGCAACCTGGAGCAAGAAGATATTGC | |
| | CACTTACTTTTGCCAACAGGGTAATACGCTTCCGTACACGTTCGGAGGGGGG | |
| | ACTAAGTTGGAAATAACAGGCTCCACCTCTGGATCCGGCAAGCCCGGATCTG | |
| | GCGAGGGATCCACCAAGGGCGAGGTGAAACTGCAGGAGTCAGGACCTGGCCT | |
| | GGTGGCGCCCTCACAGAGCCTGTCCGTCACATGCACTGTCTCAGGGGTCTCA | |
| | TTACCCGACTATGGTGTAAGCTGGATTCGCCAGCCTCCACGAAAGGGTCTGG | |
| | AGTGGCTGGGAGTAATATGGGGTAGTGAAACCACATACTATAATTCAGCTCT | |
| | CAAATCCAGACTGACCATCATCAAGGACAACTCCAAGAGCCAAGTTTTCTTA | |
| | AAAATGAACAGTCTGCAAACTGATGACACAGCCATTTACTACTGTGCCAAAC | |
| | ATTATTACTACGGTGGTAGCTATGCTATGGACTACTGGGGTCAAGGAACCTC | |
| | AGTCACCGTCTCCTCAGCGGCCGCAGGTACCACCACAACGCCCGCTCCTCGG | |
| | CCACCGACGCCAGCGCCAACTATTGCGAGTCAGCCTCTCAGTCTGCGACCTG | |
| | AGGCTTGTCGACCAGCAGCCGGAGGCGCAGTGCACACGAGGGGGCTGGACTT | |
| | CGCCTGTGATAGAAGACCTCCTTCTAAGCCCTTTTGGGTGCTGGTGGTGGTT | |
| | GGTGGAGTCCTGGCTTGCTATAGCTTGCTAGTAACAGTGGCCTTTATTATTT | |
| | TCTGGGTGAGGAGTAAGAGGAGCAGGCTCCTGCACAGTGACTACATGAACAT | |
| | GACTCCCAGGCGGCCCGGACCCACCCGCAAGCATTACCAGCCCTATGCCCCA | |
| | CCACGCGACTTCGCAGCCTATCGCTCCGCTAGCCTGAGAGTGAAGTTCAGCA | |
| | GGAGCGCAGACGCCCCCGCGTACCAGCAGGGCCAGAACCAGCTCTATAACGA | |
| | GCTCAATCTAGGACGAAGAGAGGAGTACGATGTTTTGGACAAGAGACGTGGC | |
| | CGGGACCCTGAGATGGGGGGAAAGCCGCAGAGAAGGAAGAACCCTCAGGAAG | |
| | GCCTGTACAATGAACTGCAGAAAGATAAGATGGCGGAGGCCTACAGTGAGAT | |
| | TGGGATGAAAGGCGAGCGCCGGAGGGGCAAGGGGCACGATGGCCTTTACCAG | |
| | GGTCTCAGTACAGCCACCAAGGACACCTACGACGCCCTTCACATGCAGGCCC | |
| | TGCCCCCTCGCGCTAGCGCCACGAACTTCTCTCTGTTAAAGCAAGCAGGCGA | |
| | CGTGGAAGAAAACCCCGGTCCCGTGAGCAAGGGCGAGGAGCTGTTCACCGGG | |
| | GTGGTGCCCATCCTGGTCGAGCTGGACGGCGACGTAAACGGCCACAAGTTCA | |
| | GCGTGTCCGGCGAGGGCGAGGGCGATGCCACCTACGGCAAGCTGACCCTGAA | |
| | GTTCATCTGCACCACCGGCAAGCTGCCCGTGCCCTGGCCCACCCTCGTGACC | |
| | ACCCTGACCTACGGCGTGCAGTGCTTCAGCCGCTACCCCGACCACATGAAGC | |
| | AGCACGACTTCTTCAAGTCCGCCATGCCCGAAGGCTACGTCCAGGAGCGCAC | |
| | CATCTTCTTCAAGGACGACGGCAACTACAAGACCCGCGCCGAGGTGAAGTTC | |
| | GAGGGCGACACCCTGGTGAACCGCATCGAGCTGAAGGGCATCGACTTCAAGG | |
| | AGGACGGCAACATCCTGGGGCACAAGCTGGAGTACAACTACAACAGCCACAA | |
| | CGTCTATATCATGGCCGACAAGCAGAAGAACGGCATCAAGGTGAACTTCAAG | |
| | ATCCGCCACAACATCGAGGACGGCAGCGTGCAGCTCGCCGACCACTACCAGC | |
| | AGAACACCCCCATCGGCGACGGCCCCGTGCTGCTGCCCGACAACCACTACCT | |
| | GAGCACCCAGTCCGCCCTGAGCAAAGACCCCAACGAGAAGCGCGATCACATG | |
| | GTCCTGCTGGAGTTCGTGACCGCCGCCGGGATCACTCTCGGCATGGACGAGC | |
| | TGTACAAGTAAATATCCAGAACCCTGACCCTGCCGTGTACCAGCTGAGAGAC | |
| | TCTAAATCCAGTGACAAGTCTGTCTGCCTATTCACCGATTTTGATTCTCAAA | |
| | CAAATGTGTCACAAAGTAAGGATTCTGATGTGTATATCACAGACAAAACTGT | |
| | GCTAGACATGAGGTCTATGGACTTCAAGAGCAACAGTGCTGTGGCCTGGAGC | |
| | AACAAATCTGACTTTGCATGTGCAAACGCCTTCAACAACAGCATTATTCCAG | |
| | AAGACACCTTCTTCCCCAGCCCAGGTAAGGGCAGCTTTGGTGCCTTCGCAGG | |
| | CTGTTTCCTTGCTTCAGGAATGGCCAGGTTCTGCCCAGAGCTCTGGTCAATG | |
| | ATGTCTAAAACTCCTCTGATTGGTGGTCTCGGCCTTATCCATTGCCA | |
| Anti CD4 | CAGGTGCAGCTGCAGGAGTCTGGAGGAGGCTTGGTGCAGCCTGGGGGGTCTC | 53 |
| VHH | TGAGACTCTCCTGTGCAGCCTCTGGATTCACATTCAGTAGCTACGACATGAG | |
| | CTGGGTCCGCCAGGCTCCGGGGAAGGGGCTCGAGTGGGTCTCAGGTATGAAT | |
| | AGTGGTGGTGGTAGAACATACTATGAAGACTCCGTGAAGGGCCGATTCACCA | |
| | TCTCCAGGTCCAACGCCAAGAACACGCTGTATCTGCAACTGAACAGCCTGAA | |
| | AACTGACGACACGGCCATGTATTACTGTGTCACATCCGACTTTGCTTACTGG | |
| | GGCCAGGGGACCCAGGTCACCGTCTCCTCATGTTGTTGTTGTTGTTGTTAA | |
| Anti CD8 | CAGGTTCAGCTGGAAGAATCTGGTGGTGGTCTGGTTCAGGCGGGTGGTTCTC | 54 |
| VHH | TGCGTCTGTCTTGCGCGGCGTCTGGTCGTACCTCTTCTAACACCTTCGTTGG | |
| | TTGGTTCCGTCAGGCGCCGGGTAAAGAACGTGAATTCGTTGCGGCGATCCGT | |
| | CGTTCTGACGACCGTACCTACTACGCGGCGTCTGTTCGTGGTCGTTTCACCA | |
| | TCTCTGGTGACTCTGCGAAAAACGTTGTTGCGCTGCAGATGTCTTCTCTGCG | |
| | TCCGGAAGACACCGCGGTTTACTACTGCGCGGCGACCCGTACCTGGCTGGTT | |
| | ACCGGTCAGTCTGACTACCCGTACTGGGGTCAGGGTACCCAGGTTACCGTTT | |
| | CTTCTTGTTGTTGTTGTTGTTGTTAA | |
| M7-Mav-CD8 | TTCTTGAAGACGAAAGGGCCTCGTGATACGCCTATTTTTATAGGTTAATGTC | 55 |
| (C8-2) full | ATGATAATAATGGTTTCTTAGACGTCAGGTGGCACTTTTCGGGGAAATGTGC | |
| vector plus | GCGGAACCCCTATTTGTTTATTTTTCTAAATACATTCAAATATGTATCCGCT | |
| insert | CATGAGACAATAACCCTGATAAATGCTTCAATAATATTGAAAAAGGAAGAGT | |
| | ATGAGTATTCAACATTTCCGTGTCGCCCTTATTCCCTTTTTTGCGGCATTTT | |
| | GCCTTCCTGTTTTTGCTCACCCAGAAACGCTGGTGAAAGTAAAAGATGCTGA | |
| | AGATCAGTTGGGTGCACGAGTGGGTTACATCGAACTGGATCTCAACAGCGGT | |
| | AAGATCCTTGAGAGTTTTCGCCCCGAAGAACGTTTTCCAATGATGAGCACTT | |
| | TTAAAGTTCTGCTATGTGGCGCGGTATTATCCCGTGTTGACGCCGGGCAAGA | |
| | GCAACTCGGTCGCCGCATACACTATTCTCAGAATGACTTGGTTGAGTACTCA | |
| | CCAGTCACAGAAAAGCATCTTACGGATGGCATGACAGTAAGAGAATTATGCA | |
| | GTGCTGCCATAACCATGAGTGATAACACTGCGGCCAACTTACTTCTGACAAC | |
| | GATCGGAGGACCGAAGGAGCTAACCGCTTTTTTGCACAACATGGGGGATCAT | |
| | GTAACTCGCCTTGATCGTTGGGAACCGGAGCTGAATGAAGCCATACCAAACG | |
| | ACGAGCGTGACACCACGATGCCTGCAGCAATGGCAACAACGTTGCGCAAACT | |
| | ATTAACTGGCGAACTACTTACTCTAGCTTCCCGGCAACAATTAATAGACTGG | |
| | ATGGAGGCGGATAAAGTTGCAGGACCACTTCTGCGCTCGGCCCTTCCGGCTG | |
| | GCTGGTTTATTGCTGATAAATCTGGAGCCGGTGAGCGTGGGTCTCGCGGTAT | |
| | CATTGCAGCACTGGGGCCAGATGGTAAGCCCTCCCGTATCGTAGTTATCTAC | |
| | ACGACGGGGAGTCAGGCAACTATGGATGAACGAAATAGACAGATCGCTGAGA | |
| | TAGGTGCCTCACTGATTAAGCATTGGTAACTGTCAGACCAAGTTTACTCATA | |
| | TATACTTTAGATTGATTTAAAACTTCATTTTTAATTTAAAAGGATCTAGGTG | |
| | AAGATCCTTTTTGATAATCTCATGACCAAAATCCCTTAACGTGAGTTTTCGT | |
| | TCCACTGAGCGTCAGACCCCGTAGAAAAGATCAAAGGATCTTCTTGAGATCC | |
| | TTTTTTTCTGCGCGTAATCTGCTGCTTGCAAACAAAAAAACCACCGCTACCA | |
| | GCGGTGGTTTGTTTGCCGGATCAAGAGCTACCAACTCTTTTTCCGAAGGTAA | |
| | CTGGCTTCAGCAGAGCGCAGATACCAAATACTGTCCTTCTAGTGTAGCCGTA | |
| | GTTAGGCCACCACTTCAAGAACTCTGTAGCACCGCCTACATACCTCGCTCTG | |
| | CTAATCCTGTTACCAGTGGCTGCTGCCAGTGGCGATAAGTCGTGTCTTACCG | |
| | GGTTGGACTCAAGACGATAGTTACCGGATAAGGCGCAGCGGTCGGGCTGAAC | |
| | GGGGGGTTCGTGCACACAGCCCAGCTTGGAGCGAACGACCTACACCGAACTG | |
| | AGATACCTACAGCGTGAGCTATGAGAAAGCGCCACGCTTCCCGAAGGGAGAA | |
| | AGGCGGACAGGTATCCGGTAAGCGGCAGGGTCGGAACAGGAGAGCGCACGAG | |
| | GGAGCTTCCAGGGGGAAACGCCTGGTATCTTTATAGTCCTGTCGGGTTTCGC | |
| | CACCTCTGACTTGAGCGTCGATTTTTGTGATGCTCGTCAGGGGGGCGGAGCC | |
| | TATGGAAAAACGCCAGCAACGCGGCCTTTTTACGGTTCCTGGCCTTTTGCTG | |
| | GCCTTTTGCTCACATGTTCTTTCCTGCGTTATCCCCTGATTCTGTGGATAAC | |
| | CGTATTACCGCCTTTGAGTGAGCTGATACCGCTCGCCGCAGCCGAACGACCG | |
| | AGCGCAGCGAGTCAGTGAGCGAGGAAGCGGAAGAGCGCCTGATGCGGTATTT | |
| | TCTCCTTACGCATCTGTGCGGTATTTCACACCGCATATATGGTGCACTCTCA | |
| | GTACAATCTGCTCTGATGCCGCATAGTTAAGCCAGTATACACTCCGCTATCG | |
| | CTACGTGACTGGGTCATGGCTGCGCCCCGACACCCGCCAACACCCGCTGACG | |
| | CGCCCTGACGGGCTTGTCTGCTCCCGGCATCCGCTTACAGACAAGCTGTGAC | |
| | CGTCTCCGGGAGCTGCATGTGTCAGAGGTTTTCACCGTCATCACCGAAACGC | |
| | GCGAGGCAGCTGCGGTAAAGCTCATCAGCGTGGTCGTGAAGCGATTCACAGA | |
| | TGTCTGCCTGTTCATCCGCGTCCAGCTCGTTGAGTTTCTCCAGAAGCGTTAA | |
| | TGTCTGGCTTCTGATAAAGCGGGCCATGTTAAGGGCGGTTTTTTCCTGTTTG | |
| | GTCACTGATGCCTCCGTGTAAGGGGGATTTCTGTTCATGGGGGTAATGATAC | |
| | CGATGAAACGAGAGAGGATGCTCACGATACGGGTTACTGATGATGAACATGC | |
| | CCGGTTACTGGAACGTTGTGAGGGTAAACAACTGGCGGTATGGATGCGGCGG | |
| | GACCAGAGAAAAATCACTCAGGGTCAATGCCAGCGCTTCGTTAATACAGATG | |
| | TAGGTGTTCCACAGGGTAGCCAGCAGCATCCTGCGATGCAGATCCGGAACAT | |
| | AATGGTGCAGGGCGCTGACTTCCGCGTTTCCAGACTTTACGAAACACGGAAA | |
| | CCGAAGACCATTCATGTTGTTGCTCAGGTCGCAGACGTTTTGCAGCAGCAGT | |
| | CGCTTCACGTTCGCTCGCGTATCGGTGATTCATTCTGCTAACCAGTAAGGCA | |
| | ACCCCGCCAGCCTAGCCGGGTCCTCAACGACAGGAGCACGATCATGCGCACC | |
| | CGTGGCCAGGACCCAACGCTGCCCGAGATGCGCCGCGTGCGGCTGCTGGAGA | |
| | TGGCGGACGCGATGGATATGTTCTGCCAAGGGTTGGTTTGCGCATTCACAGT | |
| | TCTCCGCAAGAATTGATTGGCTCCAATTCTTGGAGTGGTGAATCCGTTAGCG | |
| | AGGTGCCGCCGGCTTCCATTCAGGTCGAGGTGGCCCGGCTCCATGCACCGCG | |
| | ACGCAACGCGGGGAGGCAGACAAGGTATAGGGCGGCGCCTACAATCCATGCC | |
| | AACCCGTTCCATGTGCTCGCCGAGGCGGCATAAATCGCCGTGACGATCAGCG | |
| | GTCCAGTGATCGAAGTTAGGCTGGTAAGAGCCGCGAGCGATCCTTGAAGCTG | |
| | TCCCTGATGGTCGTCATCTACCTGCCTGGACAGCATGGCCTGCAACGCGGGC | |
| | ATCCCGATGCCGCCGGAAGCGAGAAGAATCATAATGGGGAAGGCCATCCAGC | |
| | CTCGCGTCGCGAACGCCAGCAAGACGTAGCCCAGCGCGTCGGCCGCCATGCC | |
| | GGCGATAATGGCCTGCTTCTCGCCGAAACGTTTGGTGGCGGGACCAGTGACG | |
| | AAGGCTTGAGCGAGGGCGTGCAAGATTCCGAATACCGCAAGCGACAGGCCGA | |
| | TCATCGTCGCGCTCCAGCGAAAGCGGTCCTCGCCGAAAATGACCCAGAGCGC | |
| | TGCCGGCACCTGTCCTACGAGTTGCATGATAAAGAAGACAGTCATAAGTGCG | |
| | GCGACGATAGTCATGCCCCGCGCCCACCGGAAGGAGCTGACTGGGTTGAAGG | |
| | CTCTCAAGGGCATCGGTCGAGATCCCGGTGCCTAATGAGTGAGCTAACTTAC | |
| | ATTAATTGCGTTGCGCTCACTGCCCGCTTTCCAGTCGGGAAACCTGTCGTGC | |
| | CAGCTGCATTAATGAATCGGCCAACGCGCGGGGAGAGGCGGTTTGCGTATTG | |
| | GGCGCCAGGGTGGTTTTTCTTTTCACCAGTGAGACGGGCAACAGCTGATTGC | |
| | CCTTCACCGCCTGGCCCTGAGAGAGTTGCAGCAAGCGGTCCACGCTGGTTTG | |
| | CCCCAGCAGGCGAAAATCCTGTTTGATGGTGGTTAACGGCGGGATATAACAT | |
| | GAGCTGTCTTCGGTATCGTCGTATCCCACTACCGAGATATCCGCACCAACGC | |
| | GCAGCCCGGACTCGGTAATGGCGCGCATTGCGCCCAGCGCCATCTGATCGTT | |
| | GGCAACCAGCATCGCAGTGGGAACGATGCCCTCATTCAGCATTTGCATGGTT | |
| | TGTTGAAAACCGGACATGGCACTCCAGTCGCCTTCCCGTTCCGCTATCGGCT | |
| | GAATTTGATTGCGAGTGAGATATTTATGCCAGCCAGCCAGACGCAGACGCGC | |
| | CGAGACAGAACTTAATGGGCCCGCTAACAGCGCGATTTGCTGGTGACCCAAT | |
| | GCGACCAGATGCTCCACGCCCAGTCGCGTACCGTCTTCATGGGAGAAAATAA | |
| | TACTGTTGATGGGTGTCTGGTCAGAGACATCAAGAAATAACGCCGGAACATT | |
| | AGTGCAGGCAGCTTCCACAGCAATGGCATCCTGGTCATCCAGCGGATAGTTA | |
| | ATGATCAGCCCACTGACGCGTTGCGCGAGAAGATTGTGCACCGCCGCTTTAC | |
| | AGGCTTCGACGCCGCTTCGTTCTACCATCGACACCACCACGCTGGCACCCAG | |
| | TTGATCGGCGCGAGATTTAATCGCCGCGACAATTTGCGACGGCGCGTGCAGG | |
| | GCCAGACTGGAGGTGGCAACGCCAATCAGCAACGACTGTTTGCCCGCCAGTT | |
| | GTTGTGCCACGCGGTTGGGAATGTAATTCAGCTCCGCCATCGCCGCTTCCAC | |
| | TTTTTCCCGCGTTTTCGCAGAAACGTGGCTGGCCTGGTTCACCACGCGGGAA | |
| | ACGGTCTGATAAGAGACACCGGCATACTCTGCGACATCGTATAACGTTACTG | |
| | GTTTCACATTCACCACCCTGAATTGACTCTCTTCCGGGCGCTATCATGCCAT | |
| | ACCGCGAAAGGTTTTGCGCCATTCGATGGTGTCCGGGATCTCGACGCTCTCC | |
| | CTTATGCGACTCCTGCATTAGGAAGCAGCCCAGTAGTAGGTTGAGGCCGTTG | |
| | AGCACCGCCGCCGCAAGGAATGGTGCATGCAAGGAGATGGCGCCCAACAGTC | |
| | CCCCGGCCACGGGGCCTGCCACCATACCCACGCCGAAACAAGCGCTCATGAG | |
| | CCCGAAGTGGCGAGCCCGATCTTCCCCATCGGTGATGTCGGCGATATAGGCG | |
| | CCAGCAACCGCACCTGTGGCGCCGGTGATGCCGGCCACGATGCGTCCGGCGT | |
| | AGAGGATCGAGATCTCGATCCCGCGAAATTAATACGACTCACTATAGGGGAA | |
| | TTGTGAGCGGATAACAATTCCCTCTAGAAATAATTTTGTTTAACTTTAAGAA | |
| | GGAGATATACCATGCATCATCATCATCATCACAGCAGCGGCAGAGAAAACTT | |
| | GTATTTCCAGGGCATGAACAACGGCACCAACAACTTTCAGAACTTTATTGGC | |
| | ATTAGCAGCCTGCAGAAAACCCTGCGCAACGCGCTGATTCCGACCGAAACCA | |
| | CCCAGCAGTTTATTGTGAAAAACGGCATTATTAAAGAAGATGAACTGCGCGG | |
| | CGAAAACCGCCAGATTCTGAAAGATATTATGGATGATTATTATCGCGGCTTT | |
| | ATTAGCGAAACCCTGAGCAGCATTGATGATATTGATTGGACCAGCCTGTTTG | |
| | AAAAAATGGAAATTCAGCTGAAAAACGGCGATAACAAAGATACCCTGATTAA | |
| | AGAACAGACCGAATATCGCAAAGCGATTCATAAAAAATTTGCGAACGATGAT | |
| | CGCTTTAAAAACATGTTTAGCGCGAAACTGATTAGCGATATTCTGCCGGAAT | |
| | TTGTGATTCATAACAACAACTATAGCGCGAGCGAAAAAGAAGAAAAAACCCA | |
| | GGTGATTAAACTGTTTAGCCGCTTTGCGACCAGCTTTAAAGATTATTTTAAA | |
| | AACCGCGCGAACTGCTTTAGCGCGGATGATATTAGCAGCAGCAGCTGCCATC | |
| | GCATTGTGAACGATAACGCGGAAATTTTTTTTAGCAACGCGCTGGTGTATCG | |
| | CCGCATTGTGAAAAGCCTGAGCAACGATGATATTAACAAAATTAGCGGCGAT | |
| | ATGAAAGATAGCCTGAAAGAAATGAGCCTGGAAGAAATTTATAGCTATGAAA | |
| | AATATGGCGAATTTATTACCCAGGAAGGCATTAGCTTTTATAACGATATTTG | |
| | CGGCAAAGTGAACAGCTTTATGAACCTGTATTGCCAGAAAAACAAAGAAAAC | |
| | AAAAACCTGTATAAACTGCAGAAACTGCATAAACAGATTCTGTGCATTGCGG | |
| | ATACCAGCTATGAAGTGCCGTATAAATTTGAAAGCGATGAAGAAGTGTATCA | |
| | GAGCGTGAACGGCTTTCTGGATAACATTAGCAGCAAACATATTGTGGAACGC | |
| | CTGCGCAAAATTGGCGATAACTATAACGGCTATAACCTGGATAAAATTTATA | |
| | TTGTGAGCAAATTTTATGAAAGCGTGAGCCAGAAAACCTATCGCGATTGGGA | |
| | AACCATTAACACCGCGCTGGAAATTCATTATAACAACATTCTGCCGGGCAAC | |
| | GGCAAAAGCAAAGCGGATAAAGTGAAAAAAGCGGTGAAAAACGATCTGCAGA | |
| | AAAGCATTACCGAAATTAACGAACTGGTGAGCAACTATAAACTGTGCAGCGA | |
| | TGATAACATTAAAGCGGAAACCTATATTCATGAAATTAGCCATATTCTGAAC | |
| | AACTTTGAAGCGCAGGAACTGAAATATAACCCGGAAATTCATCTGGTGGAAA | |
| | GCGAACTGAAAGCGAGCGAACTGAAAAACGTGCTGGATGTGATTATGAACGC | |
| | GTTTCATTGGTGCAGCGTGTTTATGACCGAAGAACTGGTGGATAAAGATAAC | |
| | AACTTTTATGCGGAACTGGAAGAAATTTATGATGAAATTTATCCGGTGATTA | |
| | GCCTGTATAACCTGGTGCGCAACTATGTGACCCAGAAACCGTATAGCACCAA | |
| | AAAAATTAAACTGAACTTTGGCATTCCGACCCTGGCGGATGGCTGGAGCAAA | |
| | AGCAAAGAATATAGCAACAACGCGATTATTCTGATGCGCGATAACCTGTATT | |
| | ATCTGGGCATTTTTAACGCGAAAAACAAACCGGATAAAAAAATTATTGAAGG | |
| | CAACACCAGCGAAAACAAAGGCGATTATAAAAAAATGATTTATAACCTGCTG | |
| | CCGGGCCCGAACAAAATGATTCCGAAAGTGTTTCTGAGCAGCAAAACCGGCG | |
| | TGGAAACCTATAAACCGAGCGCGTATATTCTGGAAGGCTATAAACAGAACAA | |
| | ACATATTAAAAGCAGCAAAGATTTTGATATTACCTTTTGCCATGATCTGATT | |
| | GATTATTTTAAAAACTGCATTGCGATTCATCCGGAATGGAAAAACTTTGGCT | |
| | TTGATTTTAGCGATACCAGCACCTATGAAGATATTAGCGGCTTTTATCGCGA | |
| | AGTGGAACTGCAGGGCTATAAAATTGATTGGACCTATATTAGCGAAAAAGAT | |
| | ATTGATCTGCTGCAGGAAAAAGGCCAGCTGTATCTGTTTCAGATTTATAACA | |
| | AAGATTTTAGCAAAAAAAGCACCGGCAACGATAACCTGCATACCATGTATCT | |
| | GAAAAACCTGTTTAGCGAAGAAAACCTGAAAGATATTGTGCTGAAACTGAAC | |
| | GGCGAAGCGGAAATTTTTTTTCGCAAAAGCAGCATTAAAAACCCGATTATTC | |
| | ATAAAAAAGGCAGCATTCTGGTGAACCGCACCTATGAAGCGGAAGAAAAAGA | |
| | TCAGTTTGGCAACATTCAGATTGTGCGCAAAAACATTCCGGAAAACATTTAT | |
| | CAGGAACTGTATAAATATTTTAACGATAAAAGCGATAAAGAACTGAGCGATG | |
| | AAGCGGCGAAACTGAAAAACGTGGTGGGCCATCATGAAGCGGCGACCAACAT | |
| | TGTGAAAGATTATCGCTATACCTATGATAAATATTTTCTGCATATGCCGATT | |
| | ACCATTAACTTTAAAGCGAACAAAACCGGCTTTATTAACGATCGCATTCTGC | |
| | AGTATATTGCGAAAGAAAAAGATCTGCATGTGATTGGCATTGATCGCGGCGA | |
| | ACGCAACCTGATTTATGTGAGCGTGATTGATACCTGCGGCAACATTGTGGAA | |
| | CAGAAAAGCTTTAACATTGTGAACGGCTATGATTATCAGATTAAACTGAAAC | |
| | AGCAGGAAGGCGCGCGCCAGATTGCGCGCAAAGAATGGAAAGAAATTGGCAA | |
| | AATTAAAGAAATTAAAGAAGGCTATCTGAGCCTGGTGATTCATGAAATTAGC | |
| | AAAATGGTGATTAAATATAACGCGATTATTGCGATGGAAGATCTGAGCTATG | |
| | GCTTTAAAAAAGGCCGCTTTAAAGTGGAACGCCAGGTGTATCAGAAATTTGA | |
| | AACCATGCTGATTAACAAACTGAACTATCTGGTGTTTAAAGATATTAGCATT | |
| | ACCGAAAACGGCGGCCTGCTGAAAGGCTATCAGCTGACCTATATTCCGGATA | |
| | AACTGAAAAACGTGGGCCATCAGTGCGGCTGCATTTTTTATGTGCCGGCGGC | |
| | GTATACCAGCAAAATTGATCCGACCACCGGCTTTGTGAACATTTTTAAATTT | |
| | AAAGATCTGACCGTGGATGCGAAACGCGAATTTATTAAAAAATTTGATAGCA | |
| | TTCGCTATGATAGCGAAAAAAACCTGTTTTGCTTTACCTTTGATTATAACAA | |
| | CTTTATTACCCAGAACACCGTGATGAGCAAAAGCAGCTGGAGCGTGTATACC | |
| | TATGGCGTGCGCATTAAACGCCGCTTTGTGAACGGCCGCTTTAGCAACGAAA | |
| | GCGATACCATTGATATTACCAAAGATATGGAAAAAACCCTGGAAATGACCGA | |
| | TATTAACTGGCGCGATGGCCATGATCTGCGCCAGGATATTATTGATTATGAA | |
| | ATTGTGCAGCATATTTTTGAAATTTTTCGCCTGACCGTGCAGATGCGCAACA | |
| | GCCTGAGCGAACTGGAAGATCGCGATTATGATCGCCTGATTAGCCCGGTGCT | |
| | GAACGAAAACAACATTTTTTATGATAGCGCGAAAGCGGGCGATGCGCTGCCG | |
| | AAAGATGCGGATGCGAACGGCGCGTATTGCATTGCGCTGAAAGGCCTGTATG | |
| | AAATTAAACAGATTACCGAAAACTGGAAAGAAGATGGCAAATTTAGCCGCGA | |
| | TAAACTGAAAATTAGCAACAAAGATTGGTTTGATTTTATTCAGAACAAACGC | |
| | TATCTGGGCGGCGGCGGCAGCGGCGGCGGCGGCAGCGGCGGCGGCGGCAGCG | |
| | GCGGCGGCGGCAGCGGCGGCGGCGGCAGCGGCGGCGGCGGCAGCACCAGCCC | |
| | TAAGAAAAAACGAAAAGTTGAGGATCCTAAAAAGAAACGAAAAGTTCATCAT | |
| | CATCATCATCATGAATTTGCGAGCGCGGAAGCGGGCATTACCGGCACCTGGT | |
| | ATAACCAGCATGGCAGCACCTTTACCGTGACCGCGGGCGCGGATGGCAACCT | |
| | GACCGGCCAGTATGAAAACCGCGCGCAGGGCACCGGCTGCCAGAACAGCCCG | |
| | TATACCCTGACCGGCCGCTATAACGGCACCAAACTGGAATGGCGCGTGGAAT | |
| | GGAACAACAGCACCGAAAACTGCCATAGCCGCACCGAATGGCGCGGCCAGTA | |
| | TCAGGGCGGCGCGGAAGCGCGCATTAACACCCAGTGGAACCTGACCTATGAA | |
| | GGCGGCAGCGGCCCGGCGACCGAACAGGGCCAGGATACCTTTACCAAAGTGA | |
| | AACCGAGCGCGGCGAGCGGCAGCGATTATAAAGATGATGATGATAAAAAACG | |
| | CAAAAGAAAATGCCGATATCCTATTGGCATTGACGTCAGGTGGCACTTTTCG | |
| | AGGAGATCATGCACAGGCGGCGGCGGCAGCGGCGGCGGCGGCAGCGGCGGCG | |
| | GCGGCAGCGGCGGCGGCGGCAGCGGCGGCGGCGGCAGCGGCGGCAGCCCATG | |
| | GCAGGTTCAGCTGGAAGAATCTGGTGGTGGTCTGGTTCAGGCGGGTGGTTCT | |
| | CTGCGTCTGTCTTGCGCGGCGTCTGGTCGTACCTCTTCTAACACCTTCGTTG | |
| | GTTGGTTCCGTCAGGCGCCGGGTAAAGAACGTGAATTCGTTGCGGCGATCCG | |
| | TCGTTCTGACGACCGTACCTACTACGCGGCGTCTGTTCGTGGTCGTTTCACC | |
| | ATCTCTGGTGACTCTGCGAAAAACGTTGTTGCGCTGCAGATGTCTTCTCTGC | |
| | GTCCGGAAGACACCGCGGTTTACTACTGCGCGGCGACCCGTACCTGGCTGGT | |
| | TACCGGTCAGTCTGACTACCCGTACTGGGGTCAGGGTACCCAGGTTACCGTT | |
| | TCTTCTTGTTGTTGTTGTTGTTGTTAAGCGGCCGCACTCGAGGATCCGGCTG | |
| | CTAACAAAGCCCGAAAGGAAGCTGAGTTGGCTGCTGCCACCGCTGAGCAATA | |
| | ACTAGCATAACCCCTTGGGGCCTCTAAACGGGTCTTGAGGGGTTTTTTGCTG | |
| | AAAGGAGGAACTATATCCGGATATCCCGCAAGAGGCCCGGCAGTACCGGCAT | |
| | AACCAAGCCTATGCCTACAGCATCCAGGGTGACGGTGCCGAGGATGACGATG | |
| | AGCGCATTGTTAGATTTCATACACGGTGCCTGACTGCGTTAGCAATTTAACT | |
| | GTGATAAACTACCGCATTAAAGCTTATCGATGATAAGCTGTCAAACATGAGA | |
| | A | |
| M7 Mav anti | TTCTTGAAGACGAAAGGGCCTCGTGATACGCCTATTTTTATAGGTTAATGTC | 56 |
| CD4 (DNA | ATGATAATAATGGTTTCTTAGACGTCAGGTGGCACTTTTCGGGGAAATGTGC | |
| vector + | GCGGAACCCCTATTTGTTTATTTTTCTAAATACATTCAAATATGTATCCGCT | |
| sequence) | CATGAGACAATAACCCTGATAAATGCTTCAATAATATTGAAAAAGGAAGAGT | |
| | ATGAGTATTCAACATTTCCGTGTCGCCCTTATTCCCTTTTTTGCGGCATTTT | |
| | GCCTTCCTGTTTTTGCTCACCCAGAAACGCTGGTGAAAGTAAAAGATGCTGA | |
| | AGATCAGTTGGGTGCACGAGTGGGTTACATCGAACTGGATCTCAACAGCGGT | |
| | AAGATCCTTGAGAGTTTTCGCCCCGAAGAACGTTTTCCAATGATGAGCACTT | |
| | TTAAAGTTCTGCTATGTGGCGCGGTATTATCCCGTGTTGACGCCGGGCAAGA | |
| | GCAACTCGGTCGCCGCATACACTATTCTCAGAATGACTTGGTTGAGTACTCA | |
| | CCAGTCACAGAAAAGCATCTTACGGATGGCATGACAGTAAGAGAATTATGCA | |
| | GTGCTGCCATAACCATGAGTGATAACACTGCGGCCAACTTACTTCTGACAAC | |
| | GATCGGAGGACCGAAGGAGCTAACCGCTTTTTTGCACAACATGGGGGATCAT | |
| | GTAACTCGCCTTGATCGTTGGGAACCGGAGCTGAATGAAGCCATACCAAACG | |
| | ACGAGCGTGACACCACGATGCCTGCAGCAATGGCAACAACGTTGCGCAAACT | |
| | ATTAACTGGCGAACTACTTACTCTAGCTTCCCGGCAACAATTAATAGACTGG | |
| | ATGGAGGCGGATAAAGTTGCAGGACCACTTCTGCGCTCGGCCCTTCCGGCTG | |
| | GCTGGTTTATTGCTGATAAATCTGGAGCCGGTGAGCGTGGGTCTCGCGGTAT | |
| | CATTGCAGCACTGGGGCCAGATGGTAAGCCCTCCCGTATCGTAGTTATCTAC | |
| | ACGACGGGGAGTCAGGCAACTATGGATGAACGAAATAGACAGATCGCTGAGA | |
| | TAGGTGCCTCACTGATTAAGCATTGGTAACTGTCAGACCAAGTTTACTCATA | |
| | TATACTTTAGATTGATTTAAAACTTCATTTTTAATTTAAAAGGATCTAGGTG | |
| | AAGATCCTTTTTGATAATCTCATGACCAAAATCCCTTAACGTGAGTTTTCGT | |
| | TCCACTGAGCGTCAGACCCCGTAGAAAAGATCAAAGGATCTTCTTGAGATCC | |
| | TTTTTTTCTGCGCGTAATCTGCTGCTTGCAAACAAAAAAACCACCGCTACCA | |
| | GCGGTGGTTTGTTTGCCGGATCAAGAGCTACCAACTCTTTTTCCGAAGGTAA | |
| | CTGGCTTCAGCAGAGCGCAGATACCAAATACTGTCCTTCTAGTGTAGCCGTA | |
| | GTTAGGCCACCACTTCAAGAACTCTGTAGCACCGCCTACATACCTCGCTCTG | |
| | CTAATCCTGTTACCAGTGGCTGCTGCCAGTGGCGATAAGTCGTGTCTTACCG | |
| | GGTTGGACTCAAGACGATAGTTACCGGATAAGGCGCAGCGGTCGGGCTGAAC | |
| | GGGGGGTTCGTGCACACAGCCCAGCTTGGAGCGAACGACCTACACCGAACTG | |
| | AGATACCTACAGCGTGAGCTATGAGAAAGCGCCACGCTTCCCGAAGGGAGAA | |
| | AGGCGGACAGGTATCCGGTAAGCGGCAGGGTCGGAACAGGAGAGCGCACGAG | |
| | GGAGCTTCCAGGGGGAAACGCCTGGTATCTTTATAGTCCTGTCGGGTTTCGC | |
| | CACCTCTGACTTGAGCGTCGATTTTTGTGATGCTCGTCAGGGGGGCGGAGCC | |
| | TATGGAAAAACGCCAGCAACGCGGCCTTTTTACGGTTCCTGGCCTTTTGCTG | |
| | GCCTTTTGCTCACATGTTCTTTCCTGCGTTATCCCCTGATTCTGTGGATAAC | |
| | CGTATTACCGCCTTTGAGTGAGCTGATACCGCTCGCCGCAGCCGAACGACCG | |
| | AGCGCAGCGAGTCAGTGAGCGAGGAAGCGGAAGAGCGCCTGATGCGGTATTT | |
| | TCTCCTTACGCATCTGTGCGGTATTTCACACCGCATATATGGTGCACTCTCA | |
| | GTACAATCTGCTCTGATGCCGCATAGTTAAGCCAGTATACACTCCGCTATCG | |
| | CTACGTGACTGGGTCATGGCTGCGCCCCGACACCCGCCAACACCCGCTGACG | |
| | CGCCCTGACGGGCTTGTCTGCTCCCGGCATCCGCTTACAGACAAGCTGTGAC | |
| | CGTCTCCGGGAGCTGCATGTGTCAGAGGTTTTCACCGTCATCACCGAAACGC | |
| | GCGAGGCAGCTGCGGTAAAGCTCATCAGCGTGGTCGTGAAGCGATTCACAGA | |
| | TGTCTGCCTGTTCATCCGCGTCCAGCTCGTTGAGTTTCTCCAGAAGCGTTAA | |
| | TGTCTGGCTTCTGATAAAGCGGGCCATGTTAAGGGCGGTTTTTTCCTGTTTG | |
| | GTCACTGATGCCTCCGTGTAAGGGGGATTTCTGTTCATGGGGGTAATGATAC | |
| | CGATGAAACGAGAGAGGATGCTCACGATACGGGTTACTGATGATGAACATGC | |
| | CCGGTTACTGGAACGTTGTGAGGGTAAACAACTGGCGGTATGGATGCGGCGG | |
| | GACCAGAGAAAAATCACTCAGGGTCAATGCCAGCGCTTCGTTAATACAGATG | |
| | TAGGTGTTCCACAGGGTAGCCAGCAGCATCCTGCGATGCAGATCCGGAACAT | |
| | AATGGTGCAGGGCGCTGACTTCCGCGTTTCCAGACTTTACGAAACACGGAAA | |
| | CCGAAGACCATTCATGTTGTTGCTCAGGTCGCAGACGTTTTGCAGCAGCAGT | |
| | CGCTTCACGTTCGCTCGCGTATCGGTGATTCATTCTGCTAACCAGTAAGGCA | |
| | ACCCCGCCAGCCTAGCCGGGTCCTCAACGACAGGAGCACGATCATGCGCACC | |
| | CGTGGCCAGGACCCAACGCTGCCCGAGATGCGCCGCGTGCGGCTGCTGGAGA | |
| | TGGCGGACGCGATGGATATGTTCTGCCAAGGGTTGGTTTGCGCATTCACAGT | |
| | TCTCCGCAAGAATTGATTGGCTCCAATTCTTGGAGTGGTGAATCCGTTAGCG | |
| | AGGTGCCGCCGGCTTCCATTCAGGTCGAGGTGGCCCGGCTCCATGCACCGCG | |
| | ACGCAACGCGGGGAGGCAGACAAGGTATAGGGCGGCGCCTACAATCCATGCC | |
| | AACCCGTTCCATGTGCTCGCCGAGGCGGCATAAATCGCCGTGACGATCAGCG | |
| | GTCCAGTGATCGAAGTTAGGCTGGTAAGAGCCGCGAGCGATCCTTGAAGCTG | |
| | TCCCTGATGGTCGTCATCTACCTGCCTGGACAGCATGGCCTGCAACGCGGGC | |
| | ATCCCGATGCCGCCGGAAGCGAGAAGAATCATAATGGGGAAGGCCATCCAGC | |
| | CTCGCGTCGCGAACGCCAGCAAGACGTAGCCCAGCGCGTCGGCCGCCATGCC | |
| | GGCGATAATGGCCTGCTTCTCGCCGAAACGTTTGGTGGCGGGACCAGTGACG | |
| | AAGGCTTGAGCGAGGGCGTGCAAGATTCCGAATACCGCAAGCGACAGGCCGA | |
| | TCATCGTCGCGCTCCAGCGAAAGCGGTCCTCGCCGAAAATGACCCAGAGCGC | |
| | TGCCGGCACCTGTCCTACGAGTTGCATGATAAAGAAGACAGTCATAAGTGCG | |
| | GCGACGATAGTCATGCCCCGCGCCCACCGGAAGGAGCTGACTGGGTTGAAGG | |
| | CTCTCAAGGGCATCGGTCGAGATCCCGGTGCCTAATGAGTGAGCTAACTTAC | |
| | ATTAATTGCGTTGCGCTCACTGCCCGCTTTCCAGTCGGGAAACCTGTCGTGC | |
| | CAGCTGCATTAATGAATCGGCCAACGCGCGGGGAGAGGCGGTTTGCGTATTG | |
| | GGCGCCAGGGTGGTTTTTCTTTTCACCAGTGAGACGGGCAACAGCTGATTGC | |
| | CCTTCACCGCCTGGCCCTGAGAGAGTTGCAGCAAGCGGTCCACGCTGGTTTG | |
| | CCCCAGCAGGCGAAAATCCTGTTTGATGGTGGTTAACGGCGGGATATAACAT | |
| | GAGCTGTCTTCGGTATCGTCGTATCCCACTACCGAGATATCCGCACCAACGC | |
| | GCAGCCCGGACTCGGTAATGGCGCGCATTGCGCCCAGCGCCATCTGATCGTT | |
| | GGCAACCAGCATCGCAGTGGGAACGATGCCCTCATTCAGCATTTGCATGGTT | |
| | TGTTGAAAACCGGACATGGCACTCCAGTCGCCTTCCCGTTCCGCTATCGGCT | |
| | GAATTTGATTGCGAGTGAGATATTTATGCCAGCCAGCCAGACGCAGACGCGC | |
| | CGAGACAGAACTTAATGGGCCCGCTAACAGCGCGATTTGCTGGTGACCCAAT | |
| | GCGACCAGATGCTCCACGCCCAGTCGCGTACCGTCTTCATGGGAGAAAATAA | |
| | TACTGTTGATGGGTGTCTGGTCAGAGACATCAAGAAATAACGCCGGAACATT | |
| | AGTGCAGGCAGCTTCCACAGCAATGGCATCCTGGTCATCCAGCGGATAGTTA | |
| | ATGATCAGCCCACTGACGCGTTGCGCGAGAAGATTGTGCACCGCCGCTTTAC | |
| | AGGCTTCGACGCCGCTTCGTTCTACCATCGACACCACCACGCTGGCACCCAG | |
| | TTGATCGGCGCGAGATTTAATCGCCGCGACAATTTGCGACGGCGCGTGCAGG | |
| | GCCAGACTGGAGGTGGCAACGCCAATCAGCAACGACTGTTTGCCCGCCAGTT | |
| | GTTGTGCCACGCGGTTGGGAATGTAATTCAGCTCCGCCATCGCCGCTTCCAC | |
| | TTTTTCCCGCGTTTTCGCAGAAACGTGGCTGGCCTGGTTCACCACGCGGGAA | |
| | ACGGTCTGATAAGAGACACCGGCATACTCTGCGACATCGTATAACGTTACTG | |
| | GTTTCACATTCACCACCCTGAATTGACTCTCTTCCGGGCGCTATCATGCCAT | |
| | ACCGCGAAAGGTTTTGCGCCATTCGATGGTGTCCGGGATCTCGACGCTCTCC | |
| | CTTATGCGACTCCTGCATTAGGAAGCAGCCCAGTAGTAGGTTGAGGCCGTTG | |
| | AGCACCGCCGCCGCAAGGAATGGTGCATGCAAGGAGATGGCGCCCAACAGTC | |
| | CCCCGGCCACGGGGCCTGCCACCATACCCACGCCGAAACAAGCGCTCATGAG | |
| | CCCGAAGTGGCGAGCCCGATCTTCCCCATCGGTGATGTCGGCGATATAGGCG | |
| | CCAGCAACCGCACCTGTGGCGCCGGTGATGCCGGCCACGATGCGTCCGGCGT | |
| | AGAGGATCGAGATCTCGATCCCGCGAAATTAATACGACTCACTATAGGGGAA | |
| | TTGTGAGCGGATAACAATTCCCTCTAGAAATAATTTTGTTTAACTTTAAGAA | |
| | GGAGATATACCATGCATCATCATCATCATCACAGCAGCGGCAGAGAAAACTT | |
| | GTATTTCCAGGGCATGAACAACGGCACCAACAACTTTCAGAACTTTATTGGC | |
| | ATTAGCAGCCTGCAGAAAACCCTGCGCAACGCGCTGATTCCGACCGAAACCA | |
| | CCCAGCAGTTTATTGTGAAAAACGGCATTATTAAAGAAGATGAACTGCGCGG | |
| | CGAAAACCGCCAGATTCTGAAAGATATTATGGATGATTATTATCGCGGCTTT | |
| | ATTAGCGAAACCCTGAGCAGCATTGATGATATTGATTGGACCAGCCTGTTTG | |
| | AAAAAATGGAAATTCAGCTGAAAAACGGCGATAACAAAGATACCCTGATTAA | |
| | AGAACAGACCGAATATCGCAAAGCGATTCATAAAAAATTTGCGAACGATGAT | |
| | CGCTTTAAAAACATGTTTAGCGCGAAACTGATTAGCGATATTCTGCCGGAAT | |
| | TTGTGATTCATAACAACAACTATAGCGCGAGCGAAAAAGAAGAAAAAACCCA | |
| | GGTGATTAAACTGTTTAGCCGCTTTGCGACCAGCTTTAAAGATTATTTTAAA | |
| | AACCGCGCGAACTGCTTTAGCGCGGATGATATTAGCAGCAGCAGCTGCCATC | |
| | GCATTGTGAACGATAACGCGGAAATTTTTTTTAGCAACGCGCTGGTGTATCG | |
| | CCGCATTGTGAAAAGCCTGAGCAACGATGATATTAACAAAATTAGCGGCGAT | |
| | ATGAAAGATAGCCTGAAAGAAATGAGCCTGGAAGAAATTTATAGCTATGAAA | |
| | AATATGGCGAATTTATTACCCAGGAAGGCATTAGCTTTTATAACGATATTTG | |
| | CGGCAAAGTGAACAGCTTTATGAACCTGTATTGCCAGAAAAACAAAGAAAAC | |
| | AAAAACCTGTATAAACTGCAGAAACTGCATAAACAGATTCTGTGCATTGCGG | |
| | ATACCAGCTATGAAGTGCCGTATAAATTTGAAAGCGATGAAGAAGTGTATCA | |
| | GAGCGTGAACGGCTTTCTGGATAACATTAGCAGCAAACATATTGTGGAACGC | |
| | CTGCGCAAAATTGGCGATAACTATAACGGCTATAACCTGGATAAAATTTATA | |
| | TTGTGAGCAAATTTTATGAAAGCGTGAGCCAGAAAACCTATCGCGATTGGGA | |
| | AACCATTAACACCGCGCTGGAAATTCATTATAACAACATTCTGCCGGGCAAC | |
| | GGCAAAAGCAAAGCGGATAAAGTGAAAAAAGCGGTGAAAAACGATCTGCAGA | |
| | AAAGCATTACCGAAATTAACGAACTGGTGAGCAACTATAAACTGTGCAGCGA | |
| | TGATAACATTAAAGCGGAAACCTATATTCATGAAATTAGCCATATTCTGAAC | |
| | AACTTTGAAGCGCAGGAACTGAAATATAACCCGGAAATTCATCTGGTGGAAA | |
| | GCGAACTGAAAGCGAGCGAACTGAAAAACGTGCTGGATGTGATTATGAACGC | |
| | GTTTCATTGGTGCAGCGTGTTTATGACCGAAGAACTGGTGGATAAAGATAAC | |
| | AACTTTTATGCGGAACTGGAAGAAATTTATGATGAAATTTATCCGGTGATTA | |
| | GCCTGTATAACCTGGTGCGCAACTATGTGACCCAGAAACCGTATAGCACCAA | |
| | AAAAATTAAACTGAACTTTGGCATTCCGACCCTGGCGGATGGCTGGAGCAAA | |
| | AGCAAAGAATATAGCAACAACGCGATTATTCTGATGCGCGATAACCTGTATT | |
| | ATCTGGGCATTTTTAACGCGAAAAACAAACCGGATAAAAAAATTATTGAAGG | |
| | CAACACCAGCGAAAACAAAGGCGATTATAAAAAAATGATTTATAACCTGCTG | |
| | CCGGGCCCGAACAAAATGATTCCGAAAGTGTTTCTGAGCAGCAAAACCGGCG | |
| | TGGAAACCTATAAACCGAGCGCGTATATTCTGGAAGGCTATAAACAGAACAA | |
| | ACATATTAAAAGCAGCAAAGATTTTGATATTACCTTTTGCCATGATCTGATT | |
| | GATTATTTTAAAAACTGCATTGCGATTCATCCGGAATGGAAAAACTTTGGCT | |
| | TTGATTTTAGCGATACCAGCACCTATGAAGATATTAGCGGCTTTTATCGCGA | |
| | AGTGGAACTGCAGGGCTATAAAATTGATTGGACCTATATTAGCGAAAAAGAT | |
| | ATTGATCTGCTGCAGGAAAAAGGCCAGCTGTATCTGTTTCAGATTTATAACA | |
| | AAGATTTTAGCAAAAAAAGCACCGGCAACGATAACCTGCATACCATGTATCT | |
| | GAAAAACCTGTTTAGCGAAGAAAACCTGAAAGATATTGTGCTGAAACTGAAC | |
| | GGCGAAGCGGAAATTTTTTTTCGCAAAAGCAGCATTAAAAACCCGATTATTC | |
| | ATAAAAAAGGCAGCATTCTGGTGAACCGCACCTATGAAGCGGAAGAAAAAGA | |
| | TCAGTTTGGCAACATTCAGATTGTGCGCAAAAACATTCCGGAAAACATTTAT | |
| | CAGGAACTGTATAAATATTTTAACGATAAAAGCGATAAAGAACTGAGCGATG | |
| | AAGCGGCGAAACTGAAAAACGTGGTGGGCCATCATGAAGCGGCGACCAACAT | |
| | TGTGAAAGATTATCGCTATACCTATGATAAATATTTTCTGCATATGCCGATT | |
| | ACCATTAACTTTAAAGCGAACAAAACCGGCTTTATTAACGATCGCATTCTGC | |
| | AGTATATTGCGAAAGAAAAAGATCTGCATGTGATTGGCATTGATCGCGGCGA | |
| | ACGCAACCTGATTTATGTGAGCGTGATTGATACCTGCGGCAACATTGTGGAA | |
| | CAGAAAAGCTTTAACATTGTGAACGGCTATGATTATCAGATTAAACTGAAAC | |
| | AGCAGGAAGGCGCGCGCCAGATTGCGCGCAAAGAATGGAAAGAAATTGGCAA | |
| | AATTAAAGAAATTAAAGAAGGCTATCTGAGCCTGGTGATTCATGAAATTAGC | |
| | AAAATGGTGATTAAATATAACGCGATTATTGCGATGGAAGATCTGAGCTATG | |
| | GCTTTAAAAAAGGCCGCTTTAAAGTGGAACGCCAGGTGTATCAGAAATTTGA | |
| | AACCATGCTGATTAACAAACTGAACTATCTGGTGTTTAAAGATATTAGCATT | |
| | ACCGAAAACGGCGGCCTGCTGAAAGGCTATCAGCTGACCTATATTCCGGATA | |
| | AACTGAAAAACGTGGGCCATCAGTGCGGCTGCATTTTTTATGTGCCGGCGGC | |
| | GTATACCAGCAAAATTGATCCGACCACCGGCTTTGTGAACATTTTTAAATTT | |
| | AAAGATCTGACCGTGGATGCGAAACGCGAATTTATTAAAAAATTTGATAGCA | |
| | TTCGCTATGATAGCGAAAAAAACCTGTTTTGCTTTACCTTTGATTATAACAA | |
| | CTTTATTACCCAGAACACCGTGATGAGCAAAAGCAGCTGGAGCGTGTATACC | |
| | TATGGCGTGCGCATTAAACGCCGCTTTGTGAACGGCCGCTTTAGCAACGAAA | |
| | GCGATACCATTGATATTACCAAAGATATGGAAAAAACCCTGGAAATGACCGA | |
| | TATTAACTGGCGCGATGGCCATGATCTGCGCCAGGATATTATTGATTATGAA | |
| | ATTGTGCAGCATATTTTTGAAATTTTTCGCCTGACCGTGCAGATGCGCAACA | |
| | GCCTGAGCGAACTGGAAGATCGCGATTATGATCGCCTGATTAGCCCGGTGCT | |
| | GAACGAAAACAACATTTTTTATGATAGCGCGAAAGCGGGCGATGCGCTGCCG | |
| | AAAGATGCGGATGCGAACGGCGCGTATTGCATTGCGCTGAAAGGCCTGTATG | |
| | AAATTAAACAGATTACCGAAAACTGGAAAGAAGATGGCAAATTTAGCCGCGA | |
| | TAAACTGAAAATTAGCAACAAAGATTGGTTTGATTTTATTCAGAACAAACGC | |
| | TATCTGGGCGGCGGCGGCAGCGGCGGCGGCGGCAGCGGCGGCGGCGGCAGCG | |
| | GCGGCGGCGGCAGCGGCGGCGGCGGCAGCGGCGGCGGCGGCAGCACCAGCCC | |
| | TAAGAAAAAACGAAAAGTTGAGGATCCTAAAAAGAAACGAAAAGTTCATCAT | |
| | CATCATCATCATGAATTTGCGAGCGCGGAAGCGGGCATTACCGGCACCTGGT | |
| | ATAACCAGCATGGCAGCACCTTTACCGTGACCGCGGGCGCGGATGGCAACCT | |
| | GACCGGCCAGTATGAAAACCGCGCGCAGGGCACCGGCTGCCAGAACAGCCCG | |
| | TATACCCTGACCGGCCGCTATAACGGCACCAAACTGGAATGGCGCGTGGAAT | |
| | GGAACAACAGCACCGAAAACTGCCATAGCCGCACCGAATGGCGCGGCCAGTA | |
| | TCAGGGCGGCGCGGAAGCGCGCATTAACACCCAGTGGAACCTGACCTATGAA | |
| | GGCGGCAGCGGCCCGGCGACCGAACAGGGCCAGGATACCTTTACCAAAGTGA | |
| | AACCGAGCGCGGCGAGCGGCAGCGATTATAAAGATGATGATGATAAAAAACG | |
| | CAAAAGAAAATGCCGATATCCTATTGGCATTGACGTCAGGTGGCACTTTTCG | |
| | AGGAGATCATGCACAGGCGGCGGCGGCAGCGGCGGCGGCGGCAGCGGCGGCG | |
| | GCGGCAGCGGCGGCGGCGGCAGCGGCGGCGGCGGCAGCGGCGGCAGCCCATG | |
| | GCAGGTGCAGCTGCAGGAGTCTGGAGGAGGCTTGGTGCAGCCTGGGGGGTCT | |
| | CTGAGACTCTCCTGTGCAGCCTCTGGATTCACATTCAGTAGCTACGACATGA | |
| | GCTGGGTCCGCCAGGCTCCGGGGAAGGGGCTCGAGTGGGTCTCAGGTATGAA | |
| | TAGTGGTGGTGGTAGAACATACTATGAAGACTCCGTGAAGGGCCGATTCACC | |
| | ATCTCCAGGTCCAACGCCAAGAACACGCTGTATCTGCAACTGAACAGCCTGA | |
| | AAACTGACGACACGGCCATGTATTACTGTGTCACATCCGACTTTGCTTACTG | |
| | GGGCCAGGGGACCCAGGTCACCGTCTCCTCATGTTGTTGTTGTTGTTGTTAA | |
| | GCGGCCGCACTCGAGGATCCGGCTGCTAACAAAGCCCGAAAGGAAGCTGAGT | |
| | TGGCTGCTGCCACCGCTGAGCAATAACTAGCATAACCCCTTGGGGCCTCTAA | |
| | ACGGGTCTTGAGGGGTTTTTTGCTGAAAGGAGGAACTATATCCGGATATCCC | |
| | GCAAGAGGCCCGGCAGTACCGGCATAACCAAGCCTATGCCTACAGCATCCAG | |
| | GGTGACGGTGCCGAGGATGACGATGAGCGCATTGTTAGATTTCATACACGGT | |
| | GCCTGACTGCGTTAGCAATTTAACTGTGATAAACTACCGCATTAAAGCTTAT | |
| | CGATGATAAGCTGTCAAACATGAGAA | |
| C9mAur (full | AGCGCCTGATGCGGTATTTTCTCCTTACGCATCTGTGCGGTATTTCACACCG | 57 |
| vector | CAATGGTGCACTCTCAGTACAATCTGCTCTGATGCCGCATAGTTAAGCCAGT | |
| sequence) | ATACACTCCGCTATCGCTACGTGACTGGGTCATGGCTGCGCCCCGACACCCG | |
| | CCAACACCCGCTGACGCGCCCTGACGGGCTTGTCTGCTCCCGGCATCCGCTT | |
| | ACAGACAAGCTGTGACCGTCTCCGGGAGCTGCATGTGTCAGAGGTTTTCACC | |
| | GTCATCACCGAAACGCGCGAGGCAGCTGCGGTAAAGCTCATCAGCGTGGTCG | |
| | TGAAGCGATTCACAGATGTCTGCCTGTTCATCCGCGTCCAGCTCGTTGAGTT | |
| | TCTCCAGAAGCGTTAATGTCTGGCTTCTGATAAAGCGGGCCATGTTAAGGGC | |
| | GGTTTTTTCCTGTTTGGTCACTGATGCCTCCGTGTAAGGGGGATTTCTGTTC | |
| | ATGGGGGTAATGATACCGATGAAACGAGAGAGGATGCTCACGATACGGGTTA | |
| | CTGATGATGAACATGCCCGGTTACTGGAACGTTGTGAGGGTAAACAACTGGC | |
| | GGTATGGATGCGGCGGGACCAGAGAAAAATCACTCAGGGTCAATGCCAGCGC | |
| | TTCGTTAATACAGATGTAGGTGTTCCACAGGGTAGCCAGCAGCATCCTGCGA | |
| | TGCAGATCCGGAACATAATGGTGCAGGGCGCTGACTTCCGCGTTTCCAGACT | |
| | TTACGAAACACGGAAACCGAAGACCATTCATGTTGTTGCTCAGGTCGCAGAC | |
| | GTTTTGCAGCAGCAGTCGCTTCACGTTCGCTCGCGTATCGGTGATTCATTCT | |
| | GCTAACCAGTAAGGCAACCCCGCCAGCCTAGCCGGGTCCTCAACGACAGGAG | |
| | CACGATCATGCGCACCCGTGGGGCCGCCATGCCGGCGATAATGGCCTGCTTC | |
| | TCGCCGAAACGTTTGGTGGCGGGACCAGTGACGAAGGCTTGAGCGAGGGCGT | |
| | GCAAGATTCCGAATACCGCAAGCGACAGGCCGATCATCGTCGCGCTCCAGCG | |
| | AAAGCGGTCCTCGCCGAAAATGACCCAGAGCGCTGCCGGCACCTGTCCTACG | |
| | AGTTGCATGATAAAGAAGACAGTCATAAGTGCGGCGACGATAGTCATGCCCC | |
| | GCGCCCACCGGAAGGAGCTGACTGGGTTGAAGGCTCTCAAGGGCATCGGTCG | |
| | AGATCCCGGTGCCTAATGAGTGAGCTAACTTACATTAATTGCGTTGCGCTCA | |
| | CTGCCCGCTTTCCAGTCGGGAAACCTGTCGTGCCAGCTGCATTAATGAATCG | |
| | GCCAACGCGCGGGGAGAGGCGGTTTGCGTATTGGGCGCCAGGGTGGTTTTTC | |
| | TTTTCACCAGTGAGACGGGCAACAGCTGATTGCCCTTCACCGCCTGGCCCTG | |
| | AGAGAGTTGCAGCAAGCGGTCCACGCTGGTTTGCCCCAGCAGGCGAAAATCC | |
| | TGTTTGATGGTGGTTAACGGCGGGATATAACATGAGCTGTCTTCGGTATCGT | |
| | CGTATCCCACTACCGAGATATCCGCACCAACGCGCAGCCCGGACTCGGTAAT | |
| | GGCGCGCATTGCGCCCAGCGCCATCTGATCGTTGGCAACCAGCATCGCAGTG | |
| | GGAACGATGCCCTCATTCAGCATTTGCATGGTTTGTTGAAAACCGGACATGG | |
| | CACTCCAGTCGCCTTCCCGTTCCGCTATCGGCTGAATTTGATTGCGAGTGAG | |
| | ATATTTATGCCAGCCAGCCAGACGCAGACGCGCCGAGACAGAACTTAATGGG | |
| | CCCGCTAACAGCGCGATTTGCTGGTGACCCAATGCGACCAGATGCTCCACGC | |
| | CCAGTCGCGTACCGTCTTCATGGGAGAAAATAATACTGTTGATGGGTGTCTG | |
| | GTCAGAGACATCAAGAAATAACGCCGGAACATTAGTGCAGGCAGCTTCCACA | |
| | GCAATGGCATCCTGGTCATCCAGCGGATAGTTAATGATCAGCCCACTGACGC | |
| | GTTGCGCGAGAAGATTGTGCACCGCCGCTTTACAGGCTTCGACGCCGCTTCG | |
| | TTCTACCATCGACACCACCACGCTGGCACCCAGTTGATCGGCGCGAGATTTA | |
| | ATCGCCGCGACAATTTGCGACGGCGCGTGCAGGGCCAGACTGGAGGTGGCAA | |
| | CGCCAATCAGCAACGACTGTTTGCCCGCCAGTTGTTGTGCCACGCGGTTGGG | |
| | AATGTAATTCAGCTCCGCCATCGCCGCTTCCACTTTTTCCCGCGTTTTCGCA | |
| | GAAACGTGGCTGGCCTGGTTCACCACGCGGGAAACGGTCTGATAAGAGACAC | |
| | CGGCATACTCTGCGACATCGTATAACGTTACTGGTTTCACATTCACCACCCT | |
| | GAATTGACTCTCTTCCGGGCGCTATCATGCCATACCGCGAAAGGTTTTGCGC | |
| | CATTCGATGGTGTCCGGGATCTCGACGCTCTCCCTTATGCGACTCCTGCATT | |
| | AGGAAGCAGCCCAGTAGTAGGTTGAGGCCGTTGAGCACCGCCGCCGCAAGGA | |
| | ATGGTGCATGCAAGGAGATGGCGCCCAACAGTCCCCCGGCCACGGGGCCTGC | |
| | CACCATACCCACGCCGAAACAAGCGCTCATGAGCCCGAAGTGGCGAGCCCGA | |
| | TCTTCCCCATCGGTGATGTCGGCGATATAGGCGCCAGCAACCGCACCTGTGG | |
| | CGCCGGTGATGCCGGCCACGATGCGTCCGGCGTAGAGGATCGAGATCTCGAT | |
| | CCCGCGAAATTAATACGACTCACTATAGGGGAATTGTGAGCGGATAACAATT | |
| | CCCCTCTAGAAATAATTTTGTTTAACTTTAAGAAGGAGATATACATATGCAC | |
| | CATCACCATCACCATGGAAAAATCGAAGAAGGTAAACTGGTAATCTGGATTA | |
| | ACGGCGATAAAGGCTATAACGGTCTCGCTGAAGTCGGTAAGAAATTCGAGAA | |
| | AGATACCGGAATTAAAGTCACCGTTGAGCATCCGGATAAACTGGAAGAGAAA | |
| | TTCCCACAGGTTGCGGCAACTGGCGATGGCCCTGACATTATCTTCTGGGCAC | |
| | ACGACCGCTTTGGTGGCTACGCTCAATCTGGCCTGTTGGCTGAAATCACCCC | |
| | GGACAAAGCGTTCCAGGACAAGCTGTATCCGTTTACCTGGGATGCCGTACGT | |
| | TACAACGGCAAGCTGATTGCTTACCCGATCGCTGTTGAAGCGTTATCGCTGA | |
| | TTTATAACAAAGATCTGCTGCCGAACCCGCCAAAAACCTGGGAAGAGATCCC | |
| | GGCGCTGGATAAAGAACTGAAAGCGAAAGGTAAGAGCGCGCTGATGTTCAAC | |
| | CTGCAAGAACCGTACTTCACCTGGCCGCTGATTGCTGCTGACGGGGGTTATG | |
| | CGTTCAAGTATGAAAACGGCAAGTACGACATTAAAGACGTGGGCGTGGATAA | |
| | CGCTGGCGCGAAAGCGGGTCTGACCTTCCTGGTTGACCTGATTAAAAACAAA | |
| | CACATGAATGCAGACACCGATTACTCCATCGCAGAAGCTGCCTTTAATAAAG | |
| | GCGAAACAGCGATGACCATCAACGGCCCGTGGGCATGGTCCAACATCGACAC | |
| | CAGCAAAGTGAATTATGGTGTAACGGTACTGCCGACCTTCAAGGGTCAACCA | |
| | TCCAAACCGTTCGTTGGCGTGCTGAGCGCAGGTATTAACGCCGCCAGTCCGA | |
| | ACAAAGAGCTGGCAAAAGAGTTCCTCGAAAACTATCTGCTGACTGATGAAGG | |
| | TCTGGAAGCGGTTAATAAAGACAAACCGCTGGGTGCCGTAGCGCTGAAGTCT | |
| | TACGAGGAAGAGTTGGCGAAAGATCCACGTATTGCCGCCACTATGGAAAACG | |
| | CCCAGAAAGGTGAAATCATGCCGAACATCCCGCAGATGTCCGCTTTCTGGTA | |
| | TGCCGTGCGTACTGCGGTGATCAACGCCGCCAGCGGTCGTCAGACTGTCGAT | |
| | GAAGCCCTGAAAGACGCGCAGACTAATTCGAGCTCGAACAACAACAACACTA | |
| | GTGAAAACCTGTATTTCCAGGGAGCAGCCTCGATGGATAAGAAATACTCAAT | |
| | AGGCTTAGATATCGGCACAAATAGCGTCGGATGGGCGGTGATCACTGATGAA | |
| | TATAAGGTTCCGTCTAAAAAGTTCAAGGTTCTGGGAAATACAGACCGCCACA | |
| | GTATCAAAAAAAATCTTATAGGGGCTCTTTTATTTGACAGTGGAGAGACAGC | |
| | GGAAGCGACTCGTCTCAAACGGACAGCTCGTAGAAGGTATACACGTCGGAAG | |
| | AATCGTATTTGTTATCTACAGGAGATTTTTTCAAATGAGATGGCGAAAGTAG | |
| | ATGATAGTTTCTTTCATCGACTTGAAGAGTCTTTTTTGGTGGAAGAAGACAA | |
| | GAAGCATGAACGTCATCCTATTTTTGGAAATATAGTAGATGAAGTTGCTTAT | |
| | CATGAGAAATATCCAACTATCTATCATCTGCGAAAAAAATTGGTAGATTCTA | |
| | CTGATAAAGCGGATTTGCGCTTAATCTATTTGGCCTTAGCGCATATGATTAA | |
| | GTTTCGTGGTCATTTTTTGATTGAGGGAGATTTAAATCCTGATAATAGTGAT | |
| | GTGGACAAACTATTTATCCAGTTGGTACAAACCTACAATCAATTATTTGAAG | |
| | AAAACCCTATTAACGCAAGTGGAGTAGATGCTAAAGCGATTCTTTCTGCACG | |
| | ATTGAGTAAATCAAGACGATTAGAAAATCTCATTGCTCAGCTCCCCGGTGAG | |
| | AAGAAAAATGGCTTATTTGGGAATCTCATTGCTTTGTCATTGGGTTTGACCC | |
| | CTAATTTTAAATCAAATTTTGATTTGGCAGAAGATGCTAAATTACAGCTTTC | |
| | AAAAGATACTTACGATGATGATTTAGATAATTTATTGGCGCAAATTGGAGAT | |
| | CAATATGCTGATTTGTTTTTGGCAGCTAAGAATTTATCAGATGCTATTTTAC | |
| | TTTCAGATATCCTAAGAGTAAATACTGAAATAACTAAGGCTCCCCTATCAGC | |
| | TTCAATGATTAAACGCTACGATGAACATCATCAAGACTTGACTCTTTTAAAA | |
| | GCTTTAGTTCGACAACAACTTCCAGAAAAGTATAAAGAAATCTTTTTTGATC | |
| | AATCAAAAAACGGATATGCAGGTTATATTGATGGGGGAGCTAGCCAAGAAGA | |
| | ATTTTATAAATTTATCAAACCAATTTTAGAAAAAATGGATGGTACTGAGGAA | |
| | TTATTGGTGAAACTAAATCGTGAAGATTTGCTGCGCAAGCAACGGACCTTTG | |
| | ACAACGGCTCTATTCCCCATCAAATTCACTTGGGTGAGCTGCATGCTATTTT | |
| | GAGAAGACAAGAAGACTTTTATCCATTTTTAAAAGACAATCGTGAGAAGATT | |
| | GAAAAAATCTTGACTTTTCGAATTCCTTATTATGTTGGTCCATTGGCGCGTG | |
| | GCAATAGTCGTTTTGCATGGATGACTCGGAAGTCTGAAGAAACAATTACCCC | |
| | ATGGAATTTTGAAGAAGTTGTCGATAAAGGTGCTTCAGCTCAATCATTTATT | |
| | GAACGCATGACAAACTTTGATAAAAATCTTCCAAATGAAAAAGTACTACCAA | |
| | AACATAGTTTGCTTTATGAGTATTTTACGGTTTATAACGAATTGACAAAGGT | |
| | CAAATATGTTACTGAAGGAATGCGAAAACCAGCATTTCTTTCAGGTGAACAG | |
| | AAGAAAGCCATTGTTGATTTACTCTTCAAAACAAATCGAAAAGTAACCGTTA | |
| | AGCAATTAAAAGAAGATTATTTCAAAAAAATAGAATGTTTTGATAGTGTTGA | |
| | AATTTCAGGAGTTGAAGATAGATTTAATGCTTCATTAGGTACCTACCATGAT | |
| | TTGCTAAAAATTATTAAAGATAAAGATTTTTTGGATAATGAAGAAAATGAAG | |
| | ATATCTTAGAGGATATTGTTTTAACATTGACCTTATTTGAAGATAGGGAGAT | |
| | GATTGAGGAAAGACTTAAAACATATGCTCACCTCTTTGATGATAAGGTGATG | |
| | AAACAGCTTAAACGTCGCCGTTATACTGGTTGGGGACGTTTGTCTCGAAAAT | |
| | TGATTAATGGTATTAGGGATAAGCAATCTGGCAAAACAATATTAGATTTTTT | |
| | GAAATCAGATGGTTTTGCCAATCGCAATTTTATGCAGCTGATCCATGATGAT | |
| | AGTTTGACATTTAAAGAAGACATTCAAAAAGCACAAGTGTCTGGACAAGGCG | |
| | ATAGTTTACATGAACATATTGCAAATTTAGCTGGTAGCCCTGCTATTAAAAA | |
| | AGGTATTTTACAGACTGTAAAAGTTGTTGATGAATTGGTCAAAGTAATGGGG | |
| | CGGCATAAGCCAGAAAATATCGTTATTGAAATGGCACGTGAAAATCAGACAA | |
| | CTCAAAAGGGCCAGAAAAATTCGCGAGAGCGTATGAAACGAATCGAAGAAGG | |
| | TATCAAAGAATTAGGAAGTCAGATTCTTAAAGAGCATCCTGTTGAAAATACT | |
| | CAATTGCAAAATGAAAAGCTCTATCTCTATTATCTCCAAAATGGAAGAGACA | |
| | TGTATGTGGACCAAGAATTAGATATTAATCGTTTAAGTGATTATGATGTCGA | |
| | TCACATTGTTCCACAAAGTTTCCTTAAAGACGATTCAATAGACAATAAGGTC | |
| | TTAACGCGTTCTGATAAAAATCGTGGTAAATCGGATAACGTTCCAAGTGAAG | |
| | AAGTAGTCAAAAAGATGAAAAACTATTGGAGACAACTTCTAAACGCCAAGTT | |
| | AATCACTCAACGTAAGTTTGATAATTTAACGAAAGCTGAACGTGGAGGTTTG | |
| | AGTGAACTTGATAAAGCTGGTTTTATCAAACGCCAATTGGTTGAAACTCGCC | |
| | AAATCACTAAGCATGTGGCACAAATTTTGGATAGTCGCATGAATACTAAATA | |
| | CGATGAAAATGATAAACTTATTCGAGAGGTTAAAGTGATTACCTTAAAATCT | |
| | AAATTAGTTTCTGACTTCCGAAAAGATTTCCAATTCTATAAAGTACGTGAGA | |
| | TTAACAATTACCATCATGCCCATGATGCGTATCTAAATGCCGTCGTTGGAAC | |
| | TGCTTTGATTAAGAAATATCCAAAACTTGAATCGGAGTTTGTCTATGGTGAT | |
| | TATAAAGTTTATGATGTTCGTAAAATGATTGCTAAGTCTGAGCAAGAAATAG | |
| | GCAAAGCAACCGCAAAATATTTCTTTTACTCTAATATCATGAACTTCTTCAA | |
| | AACAGAAATTACACTTGCAAATGGAGAGATTCGCAAACGCCCTCTAATCGAA | |
| | ACTAATGGGGAAACTGGAGAAATTGTCTGGGATAAAGGGCGAGATTTTGCCA | |
| | CAGTGCGCAAAGTATTGTCCATGCCCCAAGTCAATATTGTCAAGAAAACAGA | |
| | AGTACAGACAGGCGGATTCTCCAAGGAGTCAATTTTACCAAAAAGAAATTCG | |
| | GACAAGCTTATTGCTCGTAAAAAAGACTGGGATCCAAAAAAATATGGTGGTT | |
| | TTGATAGTCCAACGGTAGCTTATTCAGTCCTAGTGGTTGCTAAGGTGGAAAA | |
| | AGGGAAATCGAAGAAGTTAAAATCCGTTAAAGAGTTACTAGGGATCACAATT | |
| | ATGGAAAGAAGTTCCTTTGAAAAAAATCCGATTGACTTTTTAGAAGCTAAAG | |
| | GATATAAGGAAGTTAAAAAAGACTTAATCATTAAACTACCTAAATATAGTCT | |
| | TTTTGAGTTAGAAAACGGTCGTAAACGGATGCTGGCTAGTGCCGGAGAATTA | |
| | CAAAAAGGAAATGAGCTGGCTCTGCCAAGCAAATATGTGAATTTTTTATATT | |
| | TAGCTAGTCATTATGAAAAGTTGAAGGGTAGTCCAGAAGATAACGAACAAAA | |
| | ACAATTGTTTGTGGAGCAGCATAAGCATTATTTAGATGAGATTATTGAGCAA | |
| | ATCAGTGAATTTTCTAAGCGTGTTATTTTAGCAGATGCCAATTTAGATAAAG | |
| | TTCTTAGTGCATATAACAAACATAGAGACAAACCAATACGTGAACAAGCAGA | |
| | AAATATTATTCATTTATTTACGTTGACGAATCTTGGAGCTCCCGCTGCTTTT | |
| | AAATATTTTGATACAACAATTGATCGTAAACGATATACGTCTACAAAAGAAG | |
| | TTTTAGATGCCACTCTTATCCATCAATCCATCACTGGTCTTTATGAAACACG | |
| | CATTGATTTGAGTCAGCTAGGAGGTGACGGGTCACCTAAGAAAAAACGAAAA | |
| | GTTGAGGATCCTAAAAAGAAACGAAAAGTTGATAGCGGTTCAGAGACCCCAG | |
| | GAACTAGCGAGAGCGCTACACCGGAATCGGCGGAAGCGGGTATCACCGGCAC | |
| | GTGGTACAACCAGTCTGGTTCTACCTTCACCGTTACCGCGGGTGCGGACGGT | |
| | AACCTGACCGGTCAGTACGAAAACCGTGCGCAGGGCACTGGTTGCCAGAACT | |
| | CTCCGTACACCCTGACCGGTCGTTACAACGGTACCAAACTGGAATGGCGTGT | |
| | TGAATGGAACAACTCTACCGAAAACTGCCACTCTCGTACCGAATGGCGTGGT | |
| | CAGTACCAGGGTGGTGCGGAAGCGCGTATCAACACCCAGTGGAACCTGACCT | |
| | ACGAAGGTGGTTCTGGTCCGGCGACCGAACAGGGTCAGGACACCTTCACCAA | |
| | AGTTAAACCGTCTGCGGCGTCTTAAGCGGCCGCACTCGAGCACCACCACCAC | |
| | CACCACTGAGATCCGGCTGCTAACAAAGCCCGAAAGGAAGCTGAGTTGGCTG | |
| | CTGCCACCGCTGAGCAATAACTAGCATAACCCCTTGGGGCCTCTAAACGGGT | |
| | CTTGAGGGGTTTTTTGCTGAAAGGAGGAACTATATCCGGATTGGCGAATGGG | |
| | ACGCGCCCTGTAGCGGCGCATTAAGCGCGGCGGGTGTGGTGGTTACGCGCAG | |
| | CGTGACCGCTACACTTGCCAGCGCCCTAGCGCCCGCTCCTTTCGCTTTCTTC | |
| | CCTTCCTTTCTCGCCACGTTCGCCGGCTTTCCCCGTCAAGCTCTAAATCGGG | |
| | GGCTCCCTTTAGGGTTCCGATTTAGTGCTTTACGGCACCTCGACCCCAAAAA | |
| | ACTTGATTAGGGTGATGGTTCACGTAGTGGGCCATCGCCCTGATAGACGGTT | |
| | TTTCGCCCTTTGACGTTGGAGTCCACGTTCTTTAATAGTGGACTCTTGTTCC | |
| | AAACTGGAACAACACTCAACCCTATCTCGGTCTATTCTTTTGATTTATAAGG | |
| | GATTTTGCCGATTTCGGCCTATTGGTTAAAAAATGAGCTGATTTAACAAAAA | |
| | TTTAACGCGAATTTTAACAAAATATTAACGCTTACAATTTAGGTGGCACTTT | |
| | TCGGGGAAATGTGCGCGGAACCCCTATTTGTTTATTTTTCTAAATACATTCA | |
| | AATATGTATCCGCTCATGAATTAATTCTTAGAAAAACTCATCGAGCATCAAA | |
| | TGAAACTGCAATTTATTCATATCAGGATTATCAATACCATATTTTTGAAAAA | |
| | GCCGTTTCTGTAATGAAGGAGAAAACTCACCGAGGCAGTTCCATAGGATGGC | |
| | AAGATCCTGGTATCGGTCTGCGATTCCGACTCGTCCAACATCAATACAACCT | |
| | ATTAATTTCCCCTCGTCAAAAATAAGGTTATCAAGTGAGAAATCACCATGAG | |
| | TGACGACTGAATCCGGTGAGAATGGCAAAAGTTTATGCATTTCTTTCCAGAC | |
| | TTGTTCAACAGGCCAGCCATTACGCTCGTCATCAAAATCACTCGCATCAACC | |
| | AAACCGTTATTCATTCGTGATTGCGCCTGAGCGAGACGAAATACGCGATCGC | |
| | TGTTAAAAGGACAATTACAAACAGGAATCGAATGCAACCGGCGCAGGAACAC | |
| | TGCCAGCGCATCAACAATATTTTCACCTGAATCAGGATATTCTTCTAATACC | |
| | TGGAATGCTGTTTTCCCGGGGATCGCAGTGGTGAGTAACCATGCATCATCAG | |
| | GAGTACGGATAAAATGCTTGATGGTCGGAAGAGGCATAAATTCCGTCAGCCA | |
| | GTTTAGTCTGACCATCTCATCTGTAACATCATTGGCAACGCTACCTTTGCCA | |
| | TGTTTCAGAAACAACTCTGGCGCATCGGGCTTCCCATACAATCGATAGATTG | |
| | TCGCACCTGATTGCCCGACATTATCGCGAGCCCATTTATACCCATATAAATC | |
| | AGCATCCATGTTGGAATTTAATCGCGGCCTAGAGCAAGACGTTTCCCGTTGA | |
| | ATATGGCTCATAACACCCCTTGTATTACTGTTTATGTAAGCAGACAGTTTTA | |
| | TTGTTCATGACCAAAATCCCTTAACGTGAGTTTTCGTTCCACTGAGCGTCAG | |
| | ACCCCGTAGAAAAGATCAAAGGATCTTCTTGAGATCCTTTTTTTCTGCGCGT | |
| | AATCTGCTGCTTGCAAACAAAAAAACCACCGCTACCAGCGGTGGTTTGTTTG | |
| | CCGGATCAAGAGCTACCAACTCTTTTTCCGAAGGTAACTGGCTTCAGCAGAG | |
| | CGCAGATACCAAATACTGTCCTTCTAGTGTAGCCGTAGTTAGGCCACCACTT | |
| | CAAGAACTCTGTAGCACCGCCTACATACCTCGCTCTGCTAATCCTGTTACCA | |
| | GTGGCTGCTGCCAGTGGCGATAAGTCGTGTCTTACCGGGTTGGACTCAAGAC | |
| | GATAGTTACCGGATAAGGCGCAGCGGTCGGGCTGAACGGGGGGTTCGTGCAC | |
| | ACAGCCCAGCTTGGAGCGAACGACCTACACCGAACTGAGATACCTACAGCGT | |
| | GAGCTATGAGAAAGCGCCACGCTTCCCGAAGGGAGAAAGGCGGACAGGTATC | |
| | CGGTAAGCGGCAGGGTCGGAACAGGAGAGCGCACGAGGGAGCTTCCAGGGGG | |
| | AAACGCCTGGTATCTTTATAGTCCTGTCGGGTTTCGCCACCTCTGACTTGAG | |
| | CGTCGATTTTTGTGATGCTCGTCAGGGGGGCGGAGCCTATGGAAAAACGCCA | |
| | GCAACGCGGCCTTTTTACGGTTCCTGGCCTTTTGCTGGCCTTTTGCTCACAT | |
| | GTTCTTTCCTGCGTTATCCCCTGATTCTGTGGATAACCGTATTACCGCCTTT | |
| | GAGTGAGCTGATACCGCTCGCCGCAGCCGAACGACCGAGCGCAGCGAGTCAG | |
| | TGAGCGAGGAAGCGGAAG | |
| C9mC4 | AGCGCCTGATGCGGTATTTTCTCCTTACGCATCTGTGCGGTATTTCACACCG | 58 |
| | CAATGGTGCACTCTCAGTACAATCTGCTCTGATGCCGCATAGTTAAGCCAGT | |
| | ATACACTCCGCTATCGCTACGTGACTGGGTCATGGCTGCGCCCCGACACCCG | |
| | CCAACACCCGCTGACGCGCCCTGACGGGCTTGTCTGCTCCCGGCATCCGCTT | |
| | ACAGACAAGCTGTGACCGTCTCCGGGAGCTGCATGTGTCAGAGGTTTTCACC | |
| | GTCATCACCGAAACGCGCGAGGCAGCTGCGGTAAAGCTCATCAGCGTGGTCG | |
| | TGAAGCGATTCACAGATGTCTGCCTGTTCATCCGCGTCCAGCTCGTTGAGTT | |
| | TCTCCAGAAGCGTTAATGTCTGGCTTCTGATAAAGCGGGCCATGTTAAGGGC | |
| | GGTTTTTTCCTGTTTGGTCACTGATGCCTCCGTGTAAGGGGGATTTCTGTTC | |
| | ATGGGGGTAATGATACCGATGAAACGAGAGAGGATGCTCACGATACGGGTTA | |
| | CTGATGATGAACATGCCCGGTTACTGGAACGTTGTGAGGGTAAACAACTGGC | |
| | GGTATGGATGCGGCGGGACCAGAGAAAAATCACTCAGGGTCAATGCCAGCGC | |
| | TTCGTTAATACAGATGTAGGTGTTCCACAGGGTAGCCAGCAGCATCCTGCGA | |
| | TGCAGATCCGGAACATAATGGTGCAGGGCGCTGACTTCCGCGTTTCCAGACT | |
| | TTACGAAACACGGAAACCGAAGACCATTCATGTTGTTGCTCAGGTCGCAGAC | |
| | GTTTTGCAGCAGCAGTCGCTTCACGTTCGCTCGCGTATCGGTGATTCATTCT | |
| | GCTAACCAGTAAGGCAACCCCGCCAGCCTAGCCGGGTCCTCAACGACAGGAG | |
| | CACGATCATGCGCACCCGTGGGGCCGCCATGCCGGCGATAATGGCCTGCTTC | |
| | TCGCCGAAACGTTTGGTGGCGGGACCAGTGACGAAGGCTTGAGCGAGGGCGT | |
| | GCAAGATTCCGAATACCGCAAGCGACAGGCCGATCATCGTCGCGCTCCAGCG | |
| | AAAGCGGTCCTCGCCGAAAATGACCCAGAGCGCTGCCGGCACCTGTCCTACG | |
| | AGTTGCATGATAAAGAAGACAGTCATAAGTGCGGCGACGATAGTCATGCCCC | |
| | GCGCCCACCGGAAGGAGCTGACTGGGTTGAAGGCTCTCAAGGGCATCGGTCG | |
| | AGATCCCGGTGCCTAATGAGTGAGCTAACTTACATTAATTGCGTTGCGCTCA | |
| | CTGCCCGCTTTCCAGTCGGGAAACCTGTCGTGCCAGCTGCATTAATGAATCG | |
| | GCCAACGCGCGGGGAGAGGCGGTTTGCGTATTGGGCGCCAGGGTGGTTTTTC | |
| | TTTTCACCAGTGAGACGGGCAACAGCTGATTGCCCTTCACCGCCTGGCCCTG | |
| | AGAGAGTTGCAGCAAGCGGTCCACGCTGGTTTGCCCCAGCAGGCGAAAATCC | |
| | TGTTTGATGGTGGTTAACGGCGGGATATAACATGAGCTGTCTTCGGTATCGT | |
| | CGTATCCCACTACCGAGATATCCGCACCAACGCGCAGCCCGGACTCGGTAAT | |
| | GGCGCGCATTGCGCCCAGCGCCATCTGATCGTTGGCAACCAGCATCGCAGTG | |
| | GGAACGATGCCCTCATTCAGCATTTGCATGGTTTGTTGAAAACCGGACATGG | |
| | CACTCCAGTCGCCTTCCCGTTCCGCTATCGGCTGAATTTGATTGCGAGTGAG | |
| | ATATTTATGCCAGCCAGCCAGACGCAGACGCGCCGAGACAGAACTTAATGGG | |
| | CCCGCTAACAGCGCGATTTGCTGGTGACCCAATGCGACCAGATGCTCCACGC | |
| | CCAGTCGCGTACCGTCTTCATGGGAGAAAATAATACTGTTGATGGGTGTCTG | |
| | GTCAGAGACATCAAGAAATAACGCCGGAACATTAGTGCAGGCAGCTTCCACA | |
| | GCAATGGCATCCTGGTCATCCAGCGGATAGTTAATGATCAGCCCACTGACGC | |
| | GTTGCGCGAGAAGATTGTGCACCGCCGCTTTACAGGCTTCGACGCCGCTTCG | |
| | TTCTACCATCGACACCACCACGCTGGCACCCAGTTGATCGGCGCGAGATTTA | |
| | ATCGCCGCGACAATTTGCGACGGCGCGTGCAGGGCCAGACTGGAGGTGGCAA | |
| | CGCCAATCAGCAACGACTGTTTGCCCGCCAGTTGTTGTGCCACGCGGTTGGG | |
| | AATGTAATTCAGCTCCGCCATCGCCGCTTCCACTTTTTCCCGCGTTTTCGCA | |
| | GAAACGTGGCTGGCCTGGTTCACCACGCGGGAAACGGTCTGATAAGAGACAC | |
| | CGGCATACTCTGCGACATCGTATAACGTTACTGGTTTCACATTCACCACCCT | |
| | GAATTGACTCTCTTCCGGGCGCTATCATGCCATACCGCGAAAGGTTTTGCGC | |
| | CATTCGATGGTGTCCGGGATCTCGACGCTCTCCCTTATGCGACTCCTGCATT | |
| | AGGAAGCAGCCCAGTAGTAGGTTGAGGCCGTTGAGCACCGCCGCCGCAAGGA | |
| | ATGGTGCATGCAAGGAGATGGCGCCCAACAGTCCCCCGGCCACGGGGCCTGC | |
| | CACCATACCCACGCCGAAACAAGCGCTCATGAGCCCGAAGTGGCGAGCCCGA | |
| | TCTTCCCCATCGGTGATGTCGGCGATATAGGCGCCAGCAACCGCACCTGTGG | |
| | CGCCGGTGATGCCGGCCACGATGCGTCCGGCGTAGAGGATCGAGATCTCGAT | |
| | CCCGCGAAATTAATACGACTCACTATAGGGGAATTGTGAGCGGATAACAATT | |
| | CCCCTCTAGAAATAATTTTGTTTAACTTTAAGAAGGAGATATACATATGCAC | |
| | CATCACCATCACCATGGAAAAATCGAAGAAGGTAAACTGGTAATCTGGATTA | |
| | ACGGCGATAAAGGCTATAACGGTCTCGCTGAAGTCGGTAAGAAATTCGAGAA | |
| | AGATACCGGAATTAAAGTCACCGTTGAGCATCCGGATAAACTGGAAGAGAAA | |
| | TTCCCACAGGTTGCGGCAACTGGCGATGGCCCTGACATTATCTTCTGGGCAC | |
| | ACGACCGCTTTGGTGGCTACGCTCAATCTGGCCTGTTGGCTGAAATCACCCC | |
| | GGACAAAGCGTTCCAGGACAAGCTGTATCCGTTTACCTGGGATGCCGTACGT | |
| | TACAACGGCAAGCTGATTGCTTACCCGATCGCTGTTGAAGCGTTATCGCTGA | |
| | TTTATAACAAAGATCTGCTGCCGAACCCGCCAAAAACCTGGGAAGAGATCCC | |
| | GGCGCTGGATAAAGAACTGAAAGCGAAAGGTAAGAGCGCGCTGATGTTCAAC | |
| | CTGCAAGAACCGTACTTCACCTGGCCGCTGATTGCTGCTGACGGGGGTTATG | |
| | CGTTCAAGTATGAAAACGGCAAGTACGACATTAAAGACGTGGGCGTGGATAA | |
| | CGCTGGCGCGAAAGCGGGTCTGACCTTCCTGGTTGACCTGATTAAAAACAAA | |
| | CACATGAATGCAGACACCGATTACTCCATCGCAGAAGCTGCCTTTAATAAAG | |
| | GCGAAACAGCGATGACCATCAACGGCCCGTGGGCATGGTCCAACATCGACAC | |
| | CAGCAAAGTGAATTATGGTGTAACGGTACTGCCGACCTTCAAGGGTCAACCA | |
| | TCCAAACCGTTCGTTGGCGTGCTGAGCGCAGGTATTAACGCCGCCAGTCCGA | |
| | ACAAAGAGCTGGCAAAAGAGTTCCTCGAAAACTATCTGCTGACTGATGAAGG | |
| | TCTGGAAGCGGTTAATAAAGACAAACCGCTGGGTGCCGTAGCGCTGAAGTCT | |
| | TACGAGGAAGAGTTGGCGAAAGATCCACGTATTGCCGCCACTATGGAAAACG | |
| | CCCAGAAAGGTGAAATCATGCCGAACATCCCGCAGATGTCCGCTTTCTGGTA | |
| | TGCCGTGCGTACTGCGGTGATCAACGCCGCCAGCGGTCGTCAGACTGTCGAT | |
| | GAAGCCCTGAAAGACGCGCAGACTAATTCGAGCTCGAACAACAACAACACTA | |
| | GTGAAAACCTGTATTTCCAGGGAGCAGCCTCGATGGATAAGAAATACTCAAT | |
| | AGGCTTAGATATCGGCACAAATAGCGTCGGATGGGCGGTGATCACTGATGAA | |
| | TATAAGGTTCCGTCTAAAAAGTTCAAGGTTCTGGGAAATACAGACCGCCACA | |
| | GTATCAAAAAAAATCTTATAGGGGCTCTTTTATTTGACAGTGGAGAGACAGC | |
| | GGAAGCGACTCGTCTCAAACGGACAGCTCGTAGAAGGTATACACGTCGGAAG | |
| | AATCGTATTTGTTATCTACAGGAGATTTTTTCAAATGAGATGGCGAAAGTAG | |
| | ATGATAGTTTCTTTCATCGACTTGAAGAGTCTTTTTTGGTGGAAGAAGACAA | |
| | GAAGCATGAACGTCATCCTATTTTTGGAAATATAGTAGATGAAGTTGCTTAT | |
| | CATGAGAAATATCCAACTATCTATCATCTGCGAAAAAAATTGGTAGATTCTA | |
| | CTGATAAAGCGGATTTGCGCTTAATCTATTTGGCCTTAGCGCATATGATTAA | |
| | GTTTCGTGGTCATTTTTTGATTGAGGGAGATTTAAATCCTGATAATAGTGAT | |
| | GTGGACAAACTATTTATCCAGTTGGTACAAACCTACAATCAATTATTTGAAG | |
| | AAAACCCTATTAACGCAAGTGGAGTAGATGCTAAAGCGATTCTTTCTGCACG | |
| | ATTGAGTAAATCAAGACGATTAGAAAATCTCATTGCTCAGCTCCCCGGTGAG | |
| | AAGAAAAATGGCTTATTTGGGAATCTCATTGCTTTGTCATTGGGTTTGACCC | |
| | CTAATTTTAAATCAAATTTTGATTTGGCAGAAGATGCTAAATTACAGCTTTC | |
| | AAAAGATACTTACGATGATGATTTAGATAATTTATTGGCGCAAATTGGAGAT | |
| | CAATATGCTGATTTGTTTTTGGCAGCTAAGAATTTATCAGATGCTATTTTAC | |
| | TTTCAGATATCCTAAGAGTAAATACTGAAATAACTAAGGCTCCCCTATCAGC | |
| | TTCAATGATTAAACGCTACGATGAACATCATCAAGACTTGACTCTTTTAAAA | |
| | GCTTTAGTTCGACAACAACTTCCAGAAAAGTATAAAGAAATCTTTTTTGATC | |
| | AATCAAAAAACGGATATGCAGGTTATATTGATGGGGGAGCTAGCCAAGAAGA | |
| | ATTTTATAAATTTATCAAACCAATTTTAGAAAAAATGGATGGTACTGAGGAA | |
| | TTATTGGTGAAACTAAATCGTGAAGATTTGCTGCGCAAGCAACGGACCTTTG | |
| | ACAACGGCTCTATTCCCCATCAAATTCACTTGGGTGAGCTGCATGCTATTTT | |
| | GAGAAGACAAGAAGACTTTTATCCATTTTTAAAAGACAATCGTGAGAAGATT | |
| | GAAAAAATCTTGACTTTTCGAATTCCTTATTATGTTGGTCCATTGGCGCGTG | |
| | GCAATAGTCGTTTTGCATGGATGACTCGGAAGTCTGAAGAAACAATTACCCC | |
| | ATGGAATTTTGAAGAAGTTGTCGATAAAGGTGCTTCAGCTCAATCATTTATT | |
| | GAACGCATGACAAACTTTGATAAAAATCTTCCAAATGAAAAAGTACTACCAA | |
| | AACATAGTTTGCTTTATGAGTATTTTACGGTTTATAACGAATTGACAAAGGT | |
| | CAAATATGTTACTGAAGGAATGCGAAAACCAGCATTTCTTTCAGGTGAACAG | |
| | AAGAAAGCCATTGTTGATTTACTCTTCAAAACAAATCGAAAAGTAACCGTTA | |
| | AGCAATTAAAAGAAGATTATTTCAAAAAAATAGAATGTTTTGATAGTGTTGA | |
| | AATTTCAGGAGTTGAAGATAGATTTAATGCTTCATTAGGTACCTACCATGAT | |
| | TTGCTAAAAATTATTAAAGATAAAGATTTTTTGGATAATGAAGAAAATGAAG | |
| | ATATCTTAGAGGATATTGTTTTAACATTGACCTTATTTGAAGATAGGGAGAT | |
| | GATTGAGGAAAGACTTAAAACATATGCTCACCTCTTTGATGATAAGGTGATG | |
| | AAACAGCTTAAACGTCGCCGTTATACTGGTTGGGGACGTTTGTCTCGAAAAT | |
| | TGATTAATGGTATTAGGGATAAGCAATCTGGCAAAACAATATTAGATTTTTT | |
| | GAAATCAGATGGTTTTGCCAATCGCAATTTTATGCAGCTGATCCATGATGAT | |
| | AGTTTGACATTTAAAGAAGACATTCAAAAAGCACAAGTGTCTGGACAAGGCG | |
| | ATAGTTTACATGAACATATTGCAAATTTAGCTGGTAGCCCTGCTATTAAAAA | |
| | AGGTATTTTACAGACTGTAAAAGTTGTTGATGAATTGGTCAAAGTAATGGGG | |
| | CGGCATAAGCCAGAAAATATCGTTATTGAAATGGCACGTGAAAATCAGACAA | |
| | CTCAAAAGGGCCAGAAAAATTCGCGAGAGCGTATGAAACGAATCGAAGAAGG | |
| | TATCAAAGAATTAGGAAGTCAGATTCTTAAAGAGCATCCTGTTGAAAATACT | |
| | CAATTGCAAAATGAAAAGCTCTATCTCTATTATCTCCAAAATGGAAGAGACA | |
| | TGTATGTGGACCAAGAATTAGATATTAATCGTTTAAGTGATTATGATGTCGA | |
| | TCACATTGTTCCACAAAGTTTCCTTAAAGACGATTCAATAGACAATAAGGTC | |
| | TTAACGCGTTCTGATAAAAATCGTGGTAAATCGGATAACGTTCCAAGTGAAG | |
| | AAGTAGTCAAAAAGATGAAAAACTATTGGAGACAACTTCTAAACGCCAAGTT | |
| | AATCACTCAACGTAAGTTTGATAATTTAACGAAAGCTGAACGTGGAGGTTTG | |
| | AGTGAACTTGATAAAGCTGGTTTTATCAAACGCCAATTGGTTGAAACTCGCC | |
| | AAATCACTAAGCATGTGGCACAAATTTTGGATAGTCGCATGAATACTAAATA | |
| | CGATGAAAATGATAAACTTATTCGAGAGGTTAAAGTGATTACCTTAAAATCT | |
| | AAATTAGTTTCTGACTTCCGAAAAGATTTCCAATTCTATAAAGTACGTGAGA | |
| | TTAACAATTACCATCATGCCCATGATGCGTATCTAAATGCCGTCGTTGGAAC | |
| | TGCTTTGATTAAGAAATATCCAAAACTTGAATCGGAGTTTGTCTATGGTGAT | |
| | TATAAAGTTTATGATGTTCGTAAAATGATTGCTAAGTCTGAGCAAGAAATAG | |
| | GCAAAGCAACCGCAAAATATTTCTTTTACTCTAATATCATGAACTTCTTCAA | |
| | AACAGAAATTACACTTGCAAATGGAGAGATTCGCAAACGCCCTCTAATCGAA | |
| | ACTAATGGGGAAACTGGAGAAATTGTCTGGGATAAAGGGCGAGATTTTGCCA | |
| | CAGTGCGCAAAGTATTGTCCATGCCCCAAGTCAATATTGTCAAGAAAACAGA | |
| | AGTACAGACAGGCGGATTCTCCAAGGAGTCAATTTTACCAAAAAGAAATTCG | |
| | GACAAGCTTATTGCTCGTAAAAAAGACTGGGATCCAAAAAAATATGGTGGTT | |
| | TTGATAGTCCAACGGTAGCTTATTCAGTCCTAGTGGTTGCTAAGGTGGAAAA | |
| | AGGGAAATCGCAGCAGTATTATAGCTATCGCACCAAGAAGTTAAAATCCGTT | |
| | AAAGAGTTACTAGGGATCACAATTATGGAAAGAAGTTCCTTTGAAAAAAATC | |
| | CGATTGACTTTTTAGAAGCTAAAGGATATAAGGAAGTTAAAAAAGACTTAAT | |
| | CATTAAACTACCTAAATATAGTCTTTTTGAGTTAGAAAACGGTCGTAAACGG | |
| | ATGCTGGCTAGTGCCGGAGAATTACAAAAAGGAAATGAGCTGGCTCTGCCAA | |
| | GCAAATATGTGAATTTTTTATATTTAGCTAGTCATTATGAAAAGTTGAAGGG | |
| | TAGTCCAGAAGATAACGAACAAAAACAATTGTTTGTGGAGCAGCATAAGCAT | |
| | TATTTAGATGAGATTATTGAGCAAATCAGTGAATTTTCTAAGCGTGTTATTT | |
| | TAGCAGATGCCAATTTAGATAAAGTTCTTAGTGCATATAACAAACATAGAGA | |
| | CAAACCAATACGTGAACAAGCAGAAAATATTATTCATTTATTTACGTTGACG | |
| | AATCTTGGAGCTCCCGCTGCTTTTAAATATTTTGATACAACAATTGATCGTA | |
| | AACGATATACGTCTACAAAAGAAGTTTTAGATGCCACTCTTATCCATCAATC | |
| | CATCACTGGTCTTTATGAAACACGCATTGATTTGAGTCAGCTAGGAGGTGAC | |
| | GGGTCACCTAAGAAAAAACGAAAAGTTGAGGATCCTAAAAAGAAACGAAAAG | |
| | TTGATAGCGGTTCAGAGACCCCAGGAACTAGCGAGAGCGCTACACCGGAATC | |
| | GGCGGAAGCGGGTATCACCGGCACGTGGTACAACCAGTCTGGTTCTACCTTC | |
| | ACCGTTACCGCGGGTGCGGACGGTAACCTGACCGGTCAGTACGAAAACCGTG | |
| | CGCAGGGCACTGGTTGCCAGAACTCTCCGTACACCCTGACCGGTCGTTACAA | |
| | CGGTACCAAACTGGAATGGCGTGTTGAATGGAACAACTCTACCGAAAACTGC | |
| | CACTCTCGTACCGAATGGCGTGGTCAGTACCAGGGTGGTGCGGAAGCGCGTA | |
| | TCAACACCCAGTGGAACCTGACCTACGAAGGTGGTTCTGGTCCGGCGACCGA | |
| | ACAGGGTCAGGACACCTTCACCAAAGTTAAACCGTCTGCGGCGTCTTAAGCG | |
| | GCCGCACTCGAGCACCACCACCACCACCACTGAGATCCGGCTGCTAACAAAG | |
| | CCCGAAAGGAAGCTGAGTTGGCTGCTGCCACCGCTGAGCAATAACTAGCATA | |
| | ACCCCTTGGGGCCTCTAAACGGGTCTTGAGGGGTTTTTTGCTGAAAGGAGGA | |
| | ACTATATCCGGATTGGCGAATGGGACGCGCCCTGTAGCGGCGCATTAAGCGC | |
| | GGCGGGTGTGGTGGTTACGCGCAGCGTGACCGCTACACTTGCCAGCGCCCTA | |
| | GCGCCCGCTCCTTTCGCTTTCTTCCCTTCCTTTCTCGCCACGTTCGCCGGCT | |
| | TTCCCCGTCAAGCTCTAAATCGGGGGCTCCCTTTAGGGTTCCGATTTAGTGC | |
| | TTTACGGCACCTCGACCCCAAAAAACTTGATTAGGGTGATGGTTCACGTAGT | |
| | GGGCCATCGCCCTGATAGACGGTTTTTCGCCCTTTGACGTTGGAGTCCACGT | |
| | TCTTTAATAGTGGACTCTTGTTCCAAACTGGAACAACACTCAACCCTATCTC | |
| | GGTCTATTCTTTTGATTTATAAGGGATTTTGCCGATTTCGGCCTATTGGTTA | |
| | AAAAATGAGCTGATTTAACAAAAATTTAACGCGAATTTTAACAAAATATTAA | |
| | CGCTTACAATTTAGGTGGCACTTTTCGGGGAAATGTGCGCGGAACCCCTATT | |
| | TGTTTATTTTTCTAAATACATTCAAATATGTATCCGCTCATGAATTAATTCT | |
| | TAGAAAAACTCATCGAGCATCAAATGAAACTGCAATTTATTCATATCAGGAT | |
| | TATCAATACCATATTTTTGAAAAAGCCGTTTCTGTAATGAAGGAGAAAACTC | |
| | ACCGAGGCAGTTCCATAGGATGGCAAGATCCTGGTATCGGTCTGCGATTCCG | |
| | ACTCGTCCAACATCAATACAACCTATTAATTTCCCCTCGTCAAAAATAAGGT | |
| | TATCAAGTGAGAAATCACCATGAGTGACGACTGAATCCGGTGAGAATGGCAA | |
| | AAGTTTATGCATTTCTTTCCAGACTTGTTCAACAGGCCAGCCATTACGCTCG | |
| | TCATCAAAATCACTCGCATCAACCAAACCGTTATTCATTCGTGATTGCGCCT | |
| | GAGCGAGACGAAATACGCGATCGCTGTTAAAAGGACAATTACAAACAGGAAT | |
| | CGAATGCAACCGGCGCAGGAACACTGCCAGCGCATCAACAATATTTTCACCT | |
| | GAATCAGGATATTCTTCTAATACCTGGAATGCTGTTTTCCCGGGGATCGCAG | |
| | TGGTGAGTAACCATGCATCATCAGGAGTACGGATAAAATGCTTGATGGTCGG | |
| | AAGAGGCATAAATTCCGTCAGCCAGTTTAGTCTGACCATCTCATCTGTAACA | |
| | TCATTGGCAACGCTACCTTTGCCATGTTTCAGAAACAACTCTGGCGCATCGG | |
| | GCTTCCCATACAATCGATAGATTGTCGCACCTGATTGCCCGACATTATCGCG | |
| | AGCCCATTTATACCCATATAAATCAGCATCCATGTTGGAATTTAATCGCGGC | |
| | CTAGAGCAAGACGTTTCCCGTTGAATATGGCTCATAACACCCCTTGTATTAC | |
| | TGTTTATGTAAGCAGACAGTTTTATTGTTCATGACCAAAATCCCTTAACGTG | |
| | AGTTTTCGTTCCACTGAGCGTCAGACCCCGTAGAAAAGATCAAAGGATCTTC | |
| | TTGAGATCCTTTTTTTCTGCGCGTAATCTGCTGCTTGCAAACAAAAAAACCA | |
| | CCGCTACCAGCGGTGGTTTGTTTGCCGGATCAAGAGCTACCAACTCTTTTTC | |
| | CGAAGGTAACTGGCTTCAGCAGAGCGCAGATACCAAATACTGTCCTTCTAGT | |
| | GTAGCCGTAGTTAGGCCACCACTTCAAGAACTCTGTAGCACCGCCTACATAC | |
| | CTCGCTCTGCTAATCCTGTTACCAGTGGCTGCTGCCAGTGGCGATAAGTCGT | |
| | GTCTTACCGGGTTGGACTCAAGACGATAGTTACCGGATAAGGCGCAGCGGTC | |
| | GGGCTGAACGGGGGGTTCGTGCACACAGCCCAGCTTGGAGCGAACGACCTAC | |
| | ACCGAACTGAGATACCTACAGCGTGAGCTATGAGAAAGCGCCACGCTTCCCG | |
| | AAGGGAGAAAGGCGGACAGGTATCCGGTAAGCGGCAGGGTCGGAACAGGAGA | |
| | GCGCACGAGGGAGCTTCCAGGGGGAAACGCCTGGTATCTTTATAGTCCTGTC | |
| | GGGTTTCGCCACCTCTGACTTGAGCGTCGATTTTTGTGATGCTCGTCAGGGG | |
| | GGCGGAGCCTATGGAAAAACGCCAGCAACGCGGCCTTTTTACGGTTCCTGGC | |
| | CTTTTGCTGGCCTTTTGCTCACATGTTCTTTCCTGCGTTATCCCCTGATTCT | |
| | GTGGATAACCGTATTACCGCCTTTGAGTGAGCTGATACCGCTCGCCGCAGCC | |
| | GAACGACCGAGCGCAGCGAGTCAGTGAGCGAGGAAGCGGAAG | |
| anti CD4 | CAGCAGTATTATAGCTATCGCACC | 59 |
| CDR (DNA) | | |
| anti CD4 | QQYYSYRT | 60 |
| CDR (a.a.) | | |
| C9mC4nab | AGCGCCTGATGCGGTATTTTCTCCTTACGCATCTGTGCGGTATTTCACACCG | 61 |
| (full | CAATGGTGCACTCTCAGTACAATCTGCTCTGATGCCGCATAGTTAAGCCAGT | |
| sequence | ATACACTCCGCTATCGCTACGTGACTGGGTCATGGCTGCGCCCCGACACCCG | |
| vector and | CCAACACCCGCTGACGCGCCCTGACGGGCTTGTCTGCTCCCGGCATCCGCTT | |
| insert) | ACAGACAAGCTGTGACCGTCTCCGGGAGCTGCATGTGTCAGAGGTTTTCACC | |
| | GTCATCACCGAAACGCGCGAGGCAGCTGCGGTAAAGCTCATCAGCGTGGTCG | |
| | TGAAGCGATTCACAGATGTCTGCCTGTTCATCCGCGTCCAGCTCGTTGAGTT | |
| | TCTCCAGAAGCGTTAATGTCTGGCTTCTGATAAAGCGGGCCATGTTAAGGGC | |
| | GGTTTTTTCCTGTTTGGTCACTGATGCCTCCGTGTAAGGGGGATTTCTGTTC | |
| | ATGGGGGTAATGATACCGATGAAACGAGAGAGGATGCTCACGATACGGGTTA | |
| | CTGATGATGAACATGCCCGGTTACTGGAACGTTGTGAGGGTAAACAACTGGC | |
| | GGTATGGATGCGGCGGGACCAGAGAAAAATCACTCAGGGTCAATGCCAGCGC | |
| | TTCGTTAATACAGATGTAGGTGTTCCACAGGGTAGCCAGCAGCATCCTGCGA | |
| | TGCAGATCCGGAACATAATGGTGCAGGGCGCTGACTTCCGCGTTTCCAGACT | |
| | TTACGAAACACGGAAACCGAAGACCATTCATGTTGTTGCTCAGGTCGCAGAC | |
| | GTTTTGCAGCAGCAGTCGCTTCACGTTCGCTCGCGTATCGGTGATTCATTCT | |
| | GCTAACCAGTAAGGCAACCCCGCCAGCCTAGCCGGGTCCTCAACGACAGGAG | |
| | CACGATCATGCGCACCCGTGGGGCCGCCATGCCGGCGATAATGGCCTGCTTC | |
| | TCGCCGAAACGTTTGGTGGCGGGACCAGTGACGAAGGCTTGAGCGAGGGCGT | |
| | GCAAGATTCCGAATACCGCAAGCGACAGGCCGATCATCGTCGCGCTCCAGCG | |
| | AAAGCGGTCCTCGCCGAAAATGACCCAGAGCGCTGCCGGCACCTGTCCTACG | |
| | AGTTGCATGATAAAGAAGACAGTCATAAGTGCGGCGACGATAGTCATGCCCC | |
| | GCGCCCACCGGAAGGAGCTGACTGGGTTGAAGGCTCTCAAGGGCATCGGTCG | |
| | AGATCCCGGTGCCTAATGAGTGAGCTAACTTACATTAATTGCGTTGCGCTCA | |
| | CTGCCCGCTTTCCAGTCGGGAAACCTGTCGTGCCAGCTGCATTAATGAATCG | |
| | GCCAACGCGCGGGGAGAGGCGGTTTGCGTATTGGGCGCCAGGGTGGTTTTTC | |
| | TTTTCACCAGTGAGACGGGCAACAGCTGATTGCCCTTCACCGCCTGGCCCTG | |
| | AGAGAGTTGCAGCAAGCGGTCCACGCTGGTTTGCCCCAGCAGGCGAAAATCC | |
| | TGTTTGATGGTGGTTAACGGCGGGATATAACATGAGCTGTCTTCGGTATCGT | |
| | CGTATCCCACTACCGAGATATCCGCACCAACGCGCAGCCCGGACTCGGTAAT | |
| | GGCGCGCATTGCGCCCAGCGCCATCTGATCGTTGGCAACCAGCATCGCAGTG | |
| | GGAACGATGCCCTCATTCAGCATTTGCATGGTTTGTTGAAAACCGGACATGG | |
| | CACTCCAGTCGCCTTCCCGTTCCGCTATCGGCTGAATTTGATTGCGAGTGAG | |
| | ATATTTATGCCAGCCAGCCAGACGCAGACGCGCCGAGACAGAACTTAATGGG | |
| | CCCGCTAACAGCGCGATTTGCTGGTGACCCAATGCGACCAGATGCTCCACGC | |
| | CCAGTCGCGTACCGTCTTCATGGGAGAAAATAATACTGTTGATGGGTGTCTG | |
| | GTCAGAGACATCAAGAAATAACGCCGGAACATTAGTGCAGGCAGCTTCCACA | |
| | GCAATGGCATCCTGGTCATCCAGCGGATAGTTAATGATCAGCCCACTGACGC | |
| | GTTGCGCGAGAAGATTGTGCACCGCCGCTTTACAGGCTTCGACGCCGCTTCG | |
| | TTCTACCATCGACACCACCACGCTGGCACCCAGTTGATCGGCGCGAGATTTA | |
| | ATCGCCGCGACAATTTGCGACGGCGCGTGCAGGGCCAGACTGGAGGTGGCAA | |
| | CGCCAATCAGCAACGACTGTTTGCCCGCCAGTTGTTGTGCCACGCGGTTGGG | |
| | AATGTAATTCAGCTCCGCCATCGCCGCTTCCACTTTTTCCCGCGTTTTCGCA | |
| | GAAACGTGGCTGGCCTGGTTCACCACGCGGGAAACGGTCTGATAAGAGACAC | |
| | CGGCATACTCTGCGACATCGTATAACGTTACTGGTTTCACATTCACCACCCT | |
| | GAATTGACTCTCTTCCGGGCGCTATCATGCCATACCGCGAAAGGTTTTGCGC | |
| | CATTCGATGGTGTCCGGGATCTCGACGCTCTCCCTTATGCGACTCCTGCATT | |
| | AGGAAGCAGCCCAGTAGTAGGTTGAGGCCGTTGAGCACCGCCGCCGCAAGGA | |
| | ATGGTGCATGCAAGGAGATGGCGCCCAACAGTCCCCCGGCCACGGGGCCTGC | |
| | CACCATACCCACGCCGAAACAAGCGCTCATGAGCCCGAAGTGGCGAGCCCGA | |
| | TCTTCCCCATCGGTGATGTCGGCGATATAGGCGCCAGCAACCGCACCTGTGG | |
| | CGCCGGTGATGCCGGCCACGATGCGTCCGGCGTAGAGGATCGAGATCTCGAT | |
| | CCCGCGAAATTAATACGACTCACTATAGGGGAATTGTGAGCGGATAACAATT | |
| | CCCCTCTAGAAATAATTTTGTTTAACTTTAAGAAGGAGATATACATATGCAC | |
| | CATCACCATCACCATGGAAAAATCGAAGAAGGTAAACTGGTAATCTGGATTA | |
| | ACGGCGATAAAGGCTATAACGGTCTCGCTGAAGTCGGTAAGAAATTCGAGAA | |
| | AGATACCGGAATTAAAGTCACCGTTGAGCATCCGGATAAACTGGAAGAGAAA | |
| | TTCCCACAGGTTGCGGCAACTGGCGATGGCCCTGACATTATCTTCTGGGCAC | |
| | ACGACCGCTTTGGTGGCTACGCTCAATCTGGCCTGTTGGCTGAAATCACCCC | |
| | GGACAAAGCGTTCCAGGACAAGCTGTATCCGTTTACCTGGGATGCCGTACGT | |
| | TACAACGGCAAGCTGATTGCTTACCCGATCGCTGTTGAAGCGTTATCGCTGA | |
| | TTTATAACAAAGATCTGCTGCCGAACCCGCCAAAAACCTGGGAAGAGATCCC | |
| | GGCGCTGGATAAAGAACTGAAAGCGAAAGGTAAGAGCGCGCTGATGTTCAAC | |
| | CTGCAAGAACCGTACTTCACCTGGCCGCTGATTGCTGCTGACGGGGGTTATG | |
| | CGTTCAAGTATGAAAACGGCAAGTACGACATTAAAGACGTGGGCGTGGATAA | |
| | CGCTGGCGCGAAAGCGGGTCTGACCTTCCTGGTTGACCTGATTAAAAACAAA | |
| | CACATGAATGCAGACACCGATTACTCCATCGCAGAAGCTGCCTTTAATAAAG | |
| | GCGAAACAGCGATGACCATCAACGGCCCGTGGGCATGGTCCAACATCGACAC | |
| | CAGCAAAGTGAATTATGGTGTAACGGTACTGCCGACCTTCAAGGGTCAACCA | |
| | TCCAAACCGTTCGTTGGCGTGCTGAGCGCAGGTATTAACGCCGCCAGTCCGA | |
| | ACAAAGAGCTGGCAAAAGAGTTCCTCGAAAACTATCTGCTGACTGATGAAGG | |
| | TCTGGAAGCGGTTAATAAAGACAAACCGCTGGGTGCCGTAGCGCTGAAGTCT | |
| | TACGAGGAAGAGTTGGCGAAAGATCCACGTATTGCCGCCACTATGGAAAACG | |
| | CCCAGAAAGGTGAAATCATGCCGAACATCCCGCAGATGTCCGCTTTCTGGTA | |
| | TGCCGTGCGTACTGCGGTGATCAACGCCGCCAGCGGTCGTCAGACTGTCGAT | |
| | GAAGCCCTGAAAGACGCGCAGACTAATTCGAGCTCGAACAACAACAACACTA | |
| | GTGAAAACCTGTATTTCCAGGGAGCAGCCTCGATGGATAAGAAATACTCAAT | |
| | AGGCTTAGATATCGGCACAAATAGCGTCGGATGGGCGGTGATCACTGATGAA | |
| | TATAAGGTTCCGTCTAAAAAGTTCAAGGTTCTGGGAAATACAGACCGCCACA | |
| | GTATCAAAAAAAATCTTATAGGGGCTCTTTTATTTGACAGTGGAGAGACAGC | |
| | GGAAGCGACTCGTCTCAAACGGACAGCTCGTAGAAGGTATACACGTCGGAAG | |
| | AATCGTATTTGTTATCTACAGGAGATTTTTTCAAATGAGATGGCGAAAGTAG | |
| | ATGATAGTTTCTTTCATCGACTTGAAGAGTCTTTTTTGGTGGAAGAAGACAA | |
| | GAAGCATGAACGTCATCCTATTTTTGGAAATATAGTAGATGAAGTTGCTTAT | |
| | CATGAGAAATATCCAACTATCTATCATCTGCGAAAAAAATTGGTAGATTCTA | |
| | CTGATAAAGCGGATTTGCGCTTAATCTATTTGGCCTTAGCGCATATGATTAA | |
| | GTTTCGTGGTCATTTTTTGATTGAGGGAGATTTAAATCCTGATAATAGTGAT | |
| | GTGGACAAACTATTTATCCAGTTGGTACAAACCTACAATCAATTATTTGAAG | |
| | AAAACCCTATTAACGCAAGTGGAGTAGATGCTAAAGCGATTCTTTCTGCACG | |
| | ATTGAGTAAATCAAGACGATTAGAAAATCTCATTGCTCAGCTCCCCGGTGAG | |
| | AAGAAAAATGGCTTATTTGGGAATCTCATTGCTTTGTCATTGGGTTTGACCC | |
| | CTAATTTTAAATCAAATTTTGATTTGGCAGAAGATGCTAAATTACAGCTTTC | |
| | AAAAGATACTTACGATGATGATTTAGATAATTTATTGGCGCAAATTGGAGAT | |
| | CAATATGCTGATTTGTTTTTGGCAGCTAAGAATTTATCAGATGCTATTTTAC | |
| | TTTCAGATATCCTAAGAGTAAATACTGAAATAACTAAGGCTCCCCTATCAGC | |
| | TTCAATGATTAAACGCTACGATGAACATCATCAAGACTTGACTCTTTTAAAA | |
| | GCTTTAGTTCGACAACAACTTCCAGAAAAGTATAAAGAAATCTTTTTTGATC | |
| | AATCAAAAAACGGATATGCAGGTTATATTGATGGGGGAGCTAGCCAAGAAGA | |
| | ATTTTATAAATTTATCAAACCAATTTTAGAAAAAATGGATGGTACTGAGGAA | |
| | TTATTGGTGAAACTAAATCGTGAAGATTTGCTGCGCAAGCAACGGACCTTTG | |
| | ACAACGGCTCTATTCCCCATCAAATTCACTTGGGTGAGCTGCATGCTATTTT | |
| | GAGAAGACAAGAAGACTTTTATCCATTTTTAAAAGACAATCGTGAGAAGATT | |
| | GAAAAAATCTTGACTTTTCGAATTCCTTATTATGTTGGTCCATTGGCGCGTG | |
| | GCAATAGTCGTTTTGCATGGATGACTCGGAAGTCTGAAGAAACAATTACCCC | |
| | ATGGAATTTTGAAGAAGTTGTCGATAAAGGTGCTTCAGCTCAATCATTTATT | |
| | GAACGCATGACAAACTTTGATAAAAATCTTCCAAATGAAAAAGTACTACCAA | |
| | AACATAGTTTGCTTTATGAGTATTTTACGGTTTATAACGAATTGACAAAGGT | |
| | CAAATATGTTACTGAAGGAATGCGAAAACCAGCATTTCTTTCAGGTGAACAG | |
| | AAGAAAGCCATTGTTGATTTACTCTTCAAAACAAATCGAAAAGTAACCGTTA | |
| | AGCAATTAAAAGAAGATTATTTCAAAAAAATAGAATGTTTTGATAGTGTTGA | |
| | AATTTCAGGAGTTGAAGATAGATTTAATGCTTCATTAGGTACCTACCATGAT | |
| | TTGCTAAAAATTATTAAAGATAAAGATTTTTTGGATAATGAAGAAAATGAAG | |
| | ATATCTTAGAGGATATTGTTTTAACATTGACCTTATTTGAAGATAGGGAGAT | |
| | GATTGAGGAAAGACTTAAAACATATGCTCACCTCTTTGATGATAAGGTGATG | |
| | AAACAGCTTAAACGTCGCCGTTATACTGGTTGGGGACGTTTGTCTCGAAAAT | |
| | TGATTAATGGTATTAGGGATAAGCAATCTGGCAAAACAATATTAGATTTTTT | |
| | GAAATCAGATGGTTTTGCCAATCGCAATTTTATGCAGCTGATCCATGATGAT | |
| | AGTTTGACATTTAAAGAAGACATTCAAAAAGCACAAGTGTCTGGACAAGGCG | |
| | ATAGTTTACATGAACATATTGCAAATTTAGCTGGTAGCCCTGCTATTAAAAA | |
| | AGGTATTTTACAGACTGTAAAAGTTGTTGATGAATTGGTCAAAGTAATGGGG | |
| | CGGCATAAGCCAGAAAATATCGTTATTGAAATGGCACGTGAAAATCAGACAA | |
| | CTCAAAAGGGCCAGAAAAATTCGCGAGAGCGTATGAAACGAATCGAAGAAGG | |
| | TATCAAAGAATTAGGAAGTCAGATTCTTAAAGAGCATCCTGTTGAAAATACT | |
| | CAATTGCAAAATGAAAAGCTCTATCTCTATTATCTCCAAAATGGAAGAGACA | |
| | TGTATGTGGACCAAGAATTAGATATTAATCGTTTAAGTGATTATGATGTCGA | |
| | TCACATTGTTCCACAAAGTTTCCTTAAAGACGATTCAATAGACAATAAGGTC | |
| | TTAACGCGTTCTGATAAAAATCGTGGTAAATCGGATAACGTTCCAAGTGAAG | |
| | AAGTAGTCAAAAAGATGAAAAACTATTGGAGACAACTTCTAAACGCCAAGTT | |
| | AATCACTCAACGTAAGTTTGATAATTTAACGAAAGCTGAACGTGGAGGTTTG | |
| | AGTGAACTTGATAAAGCTGGTTTTATCAAACGCCAATTGGTTGAAACTCGCC | |
| | AAATCACTAAGCATGTGGCACAAATTTTGGATAGTCGCATGAATACTAAATA | |
| | CGATGAAAATGATAAACTTATTCGAGAGGTTAAAGTGATTACCTTAAAATCT | |
| | AAATTAGTTTCTGACTTCCGAAAAGATTTCCAATTCTATAAAGTACGTGAGA | |
| | TTAACAATTACCATCATGCCCATGATGCGTATCTAAATGCCGTCGTTGGAAC | |
| | TGCTTTGATTAAGAAATATCCAAAACTTGAATCGGAGTTTGTCTATGGTGAT | |
| | TATAAAGTTTATGATGTTCGTAAAATGATTGCTAAGTCTGAGCAAGAAATAG | |
| | GCAAAGCAACCGCAAAATATTTCTTTTACTCTAATATCATGAACTTCTTCAA | |
| | AACAGAAATTACACTTGCAAATGGAGAGATTCGCAAACGCCCTCTAATCGAA | |
| | ACTAATGGGGAAACTGGAGAAATTGTCTGGGATAAAGGGCGAGATTTTGCCA | |
| | CAGTGCGCAAAGTATTGTCCATGCCCCAAGTCAATATTGTCAAGAAAACAGA | |
| | AGTACAGACAGGCGGATTCTCCAAGGAGTCAATTTTACCAAAAAGAAATTCG | |
| | GACAAGCTTATTGCTCGTAAAAAAGACTGGGATCCAAAAAAATATGGTGGTT | |
| | TTGATAGTCCAACGGTAGCTTATTCAGTCCTAGTGGTTGCTAAGGTGGAAAA | |
| | AGGGAAATCGCAGCAGTATTATAGCTATCGCACCAAGAAGTTAAAATCCGTT | |
| | AAAGAGTTACTAGGGATCACAATTATGGAAAGAAGTTCCTTTGAAAAAAATC | |
| | CGATTGACTTTTTAGAAGCTAAAGGATATAAGGAAGTTAAAAAAGACTTAAT | |
| | CATTAAACTACCTAAATATAGTCTTTTTGAGTTAGAAAACGGTCGTAAACGG | |
| | ATGCTGGCTAGTGCCGGAGAATTACAAAAAGGAAATGAGCTGGCTCTGCCAA | |
| | GCAAATATGTGAATTTTTTATATTTAGCTAGTCATTATGAAAAGTTGAAGGG | |
| | TAGTCCAGAAGATAACGAACAAAAACAATTGTTTGTGGAGCAGCATAAGCAT | |
| | TATTTAGATGAGATTATTGAGCAAATCAGTGAATTTTCTAAGCGTGTTATTT | |
| | TAGCAGATGCCAATTTAGATAAAGTTCTTAGTGCATATAACAAACATAGAGA | |
| | CAAACCAATACGTGAACAAGCAGAAAATATTATTCATTTATTTACGTTGACG | |
| | AATCTTGGAGCTCCCGCTGCTTTTAAATATTTTGATACAACAATTGATCGTA | |
| | AACGATATACGTCTACAAAAGAAGTTTTAGATGCCACTCTTATCCATCAATC | |
| | CATCACTGGTCTTTATGAAACACGCATTGATTTGAGTCAGCTAGGAGGTGAC | |
| | GGGTCACCTAAGAAAAAACGAAAAGTTGAGGATCCTAAAAAGAAACGAAAAG | |
| | TTGATAGCGGTTCAGAGACCCCAGGAACTAGCGAGAGCGCTACACCGGAATC | |
| | GGCGGAAGCGGGTATCACCGGCACGTGGTACAACCAGTCTGGTTCTACCTTC | |
| | ACCGTTACCGCGGGTGCGGACGGTAACCTGACCGGTCAGTACGAAAACCGTG | |
| | CGCAGGGCACTGGTTGCCAGAACTCTCCGTACACCCTGACCGGTCGTTACAA | |
| | CGGTACCAAACTGGAATGGCGTGTTGAATGGAACAACTCTACCGAAAACTGC | |
| | CACTCTCGTACCGAATGGCGTGGTCAGTACCAGGGTGGTGCGGAAGCGCGTA | |
| | TCAACACCCAGTGGAACCTGACCTACGAAGGTGGTTCTGGTCCGGCGACCGA | |
| | ACAGGGTCAGGACACCTTCACCAAAGTTAAAGGATCCCAGGTTCAGCTGGTT | |
| | CAGAGCGGTGGTGGTCTGGTTCAGGCAGGCGGTAGCCTGCGTCTGAGCTGTG | |
| | CATTTAGCGGTCGTACCTTTAGCATGTATACCATGGGTTGGTTTCGTCAGGC | |
| | ACCGGGTAAAGAACGTGAATTTGTTGCAGCAAATCGTGGTCGTGGTCTGAGT | |
| | CCGGATATTGCAGATAGCGTTAATGGTCGTTTTACCATTAGCCGTGATAATG | |
| | CCAAAAATACCCTGTACCTGCAGATGGATAGCCTGAAACCGGAAGATACCGC | |
| | AGTGTATTATTGTGCAGCAGCAAGCCGTGAAGATCCGCCTGGTTATTGGGGT | |
| | CAGGGCACCACCGTTACCGTTAGCAGCCCGAAAAAGAAACGTAAAGTGGAAG | |
| | ATCCGAAGAAAAAGCGTAAAGTTGGTGGTGGTGGCAGCCTGCCGGAAACCGG | |
| | TGGTCTGTTTGATATTATCAAGAAAATTGCCGAGAGCTTCCTGGAACATCAT | |
| | CACCATCATCATAAGCTTGATCCGGCTGCTAACAAAGCCCGAAAGGAAGCTG | |
| | AGTTGGCTGCTGCCACCGCTGAGCAATAAGATCCGGCTGCTAACAAAGCCCG | |
| | AAAGGAAGCTGAGTTGGCTGCTGCCACCGCTGAGCAATAACTAGCATAACCC | |
| | CTTGGGGCCTCTAAACGGGTCTTGAGGGGTTTTTTGCTGAAAGGAGGAACTA | |
| | TATCCGGATTGGCGAATGGGACGCGCCCTGTAGCGGCGCATTAAGCGCGGCG | |
| | GGTGTGGTGGTTACGCGCAGCGTGACCGCTACACTTGCCAGCGCCCTAGCGC | |
| | CCGCTCCTTTCGCTTTCTTCCCTTCCTTTCTCGCCACGTTCGCCGGCTTTCC | |
| | CCGTCAAGCTCTAAATCGGGGGCTCCCTTTAGGGTTCCGATTTAGTGCTTTA | |
| | CGGCACCTCGACCCCAAAAAACTTGATTAGGGTGATGGTTCACGTAGTGGGC | |
| | CATCGCCCTGATAGACGGTTTTTCGCCCTTTGACGTTGGAGTCCACGTTCTT | |
| | TAATAGTGGACTCTTGTTCCAAACTGGAACAACACTCAACCCTATCTCGGTC | |
| | TATTCTTTTGATTTATAAGGGATTTTGCCGATTTCGGCCTATTGGTTAAAAA | |
| | ATGAGCTGATTTAACAAAAATTTAACGCGAATTTTAACAAAATATTAACGCT | |
| | TACAATTTAGGTGGCACTTTTCGGGGAAATGTGCGCGGAACCCCTATTTGTT | |
| | TATTTTTCTAAATACATTCAAATATGTATCCGCTCATGAATTAATTCTTAGA | |
| | AAAACTCATCGAGCATCAAATGAAACTGCAATTTATTCATATCAGGATTATC | |
| | AATACCATATTTTTGAAAAAGCCGTTTCTGTAATGAAGGAGAAAACTCACCG | |
| | AGGCAGTTCCATAGGATGGCAAGATCCTGGTATCGGTCTGCGATTCCGACTC | |
| | GTCCAACATCAATACAACCTATTAATTTCCCCTCGTCAAAAATAAGGTTATC | |
| | AAGTGAGAAATCACCATGAGTGACGACTGAATCCGGTGAGAATGGCAAAAGT | |
| | TTATGCATTTCTTTCCAGACTTGTTCAACAGGCCAGCCATTACGCTCGTCAT | |
| | CAAAATCACTCGCATCAACCAAACCGTTATTCATTCGTGATTGCGCCTGAGC | |
| | GAGACGAAATACGCGATCGCTGTTAAAAGGACAATTACAAACAGGAATCGAA | |
| | TGCAACCGGCGCAGGAACACTGCCAGCGCATCAACAATATTTTCACCTGAAT | |
| | CAGGATATTCTTCTAATACCTGGAATGCTGTTTTCCCGGGGATCGCAGTGGT | |
| | GAGTAACCATGCATCATCAGGAGTACGGATAAAATGCTTGATGGTCGGAAGA | |
| | GGCATAAATTCCGTCAGCCAGTTTAGTCTGACCATCTCATCTGTAACATCAT | |
| | TGGCAACGCTACCTTTGCCATGTTTCAGAAACAACTCTGGCGCATCGGGCTT | |
| | CCCATACAATCGATAGATTGTCGCACCTGATTGCCCGACATTATCGCGAGCC | |
| | CATTTATACCCATATAAATCAGCATCCATGTTGGAATTTAATCGCGGCCTAG | |
| | AGCAAGACGTTTCCCGTTGAATATGGCTCATAACACCCCTTGTATTACTGTT | |
| | TATGTAAGCAGACAGTTTTATTGTTCATGACCAAAATCCCTTAACGTGAGTT | |
| | TTCGTTCCACTGAGCGTCAGACCCCGTAGAAAAGATCAAAGGATCTTCTTGA | |
| | GATCCTTTTTTTCTGCGCGTAATCTGCTGCTTGCAAACAAAAAAACCACCGC | |
| | TACCAGCGGTGGTTTGTTTGCCGGATCAAGAGCTACCAACTCTTTTTCCGAA | |
| | GGTAACTGGCTTCAGCAGAGCGCAGATACCAAATACTGTCCTTCTAGTGTAG | |
| | CCGTAGTTAGGCCACCACTTCAAGAACTCTGTAGCACCGCCTACATACCTCG | |
| | CTCTGCTAATCCTGTTACCAGTGGCTGCTGCCAGTGGCGATAAGTCGTGTCT | |
| | TACCGGGTTGGACTCAAGACGATAGTTACCGGATAAGGCGCAGCGGTCGGGC | |
| | TGAACGGGGGGTTCGTGCACACAGCCCAGCTTGGAGCGAACGACCTACACCG | |
| | AACTGAGATACCTACAGCGTGAGCTATGAGAAAGCGCCACGCTTCCCGAAGG | |
| | GAGAAAGGCGGACAGGTATCCGGTAAGCGGCAGGGTCGGAACAGGAGAGCGC | |
| | ACGAGGGAGCTTCCAGGGGGAAACGCCTGGTATCTTTATAGTCCTGTCGGGT | |
| | TTCGCCACCTCTGACTTGAGCGTCGATTTTTGTGATGCTCGTCAGGGGGGCG | |
| | GAGCCTATGGAAAAACGCCAGCAACGCGGCCTTTTTACGGTTCCTGGCCTTT | |
| | TGCTGGCCTTTTGCTCACATGTTCTTTCCTGCGTTATCCCCTGATTCTGTGG | |
| | ATAACCGTATTACCGCCTTTGAGTGAGCTGATACCGCTCGCCGCAGCCGAAC | |
| | GACCGAGCGCAGCGAGTCAGTGAGCGAGGAAGCGGAAG | |
| Anti CD4n1 | GGATCCCAGGTTCAGCTGGTTCAGAGCGGTGGTGGTCTGGTTCAGGCAGGCG | 62 |
| VHH | GTAGCCTGCGTCTGAGCTGTGCATTTAGCGGTCGTACCTTTAGCATGTATAC | |
| | CATGGGTTGGTTTCGTCAGGCACCGGGTAAAGAACGTGAATTTGTTGCAGCA | |
| | AATCGTGGTCGTGGTCTGAGTCCGGATATTGCAGATAGCGTTAATGGTCGTT | |
| | TTACCATTAGCCGTGATAATGCCAAAAATACCCTGTACCTGCAGATGGATAG | |
| | CCTGAAACCGGAAGATACCGCAGTGTATTATTGTGCAGCAGCAAGCCGTGAA | |
| | GATCCGCCTGGTTATTGGGGTCAGGGCACCACCGTTACCGTTAGCAGCCCGA | |
| | AAAAGAAACGTAAAGTGGAAGATCCGAAGAAAAAGCGTAAAGTTGGTGGTGG | |
| | TGGCAGCCTGCCGGAAACCGGTGGTCTGTTTGATATTATCAAGAAAATTGCC | |
| | GAGAGCTTCCTGGAACATCATCACCATCATCATAAGCTTGATCCGGCTGCTA | |
| | ACAAAGCCCGAAAGGAAGCTGAGTTGGCTGCTGCCACCGCTGAGCAATAA | |

### Assembly

The assembly was performed with the steps of 1) PCR amplification, 2) Gibson assembly, 3) colony screening. The PCR amplification was performed with the following steps repeated for 32 cycles: a) 98 centigrade for 30 seconds, b) 98 centigrade for 8 seconds, c) 72 centigrade for 30 seconds, followed by 5:50 min at 72 centigrade and 2 min at 82 centigrade. The PCR mix was produced by mixing 10 μL of 5×Q5 buffer, 1 μL of 10 mM dNTPs, 2 μL of 5×Q5 enhancer, 2 μL of template (C9mAur-30 ng/μL), 0.5 μL of Q5 High-Fidelity Polymerase, 1 μL of Forward and Reverse primers (25 μM), and 32.5 c of deionized water. The PCR products (11 kb) were treated with Dpn1 by adding 1 μL of Dpn1 (Thermo Scientific™) for 1 hour at 37⁰0 (twice) and then purified using the Qiagen™ purification kit. The Gibson assembly was performed with solutions having concentrations of 80 ng/μL of vector, 200 ng/μL of C4n, 290 ng/μL for L1 and 280 ng/μL for L2. The Gibson assembly master mix was obtained by mixing 1 μL of vector, 1.5 μL of 200 ng/μL of C4n and 1.5 μL of 290 ng/μL of L1 and 280 ng/μL of L2. The Gibson master mix was completed by adding water until a total volume of 20 μL. The mix was incubated for 30 min at 50° C. The transformation was performed using dh5alpha and plated on agar plates.

To perform the colony screening first a PCR amplification was performed with 18.2 μL of water, 3 μL of Taq buffer, 2.4 μL of 25 mM MgCl₂, 3 μL of 2 mM dNTPs, 2.5 μL of 10× enhancer, 0.3 μL of T7 promoter (600 μg/mL), 0.3 μL of T7 terminator (600 μg/mL), and 0.3 μL of Taq polymerase (5 u/μL). The PCR program was 94° C. for 2 min, 29 times a cycle of 95° C. for 30 sec, 50° C. for 30 sec and 68° C. for 1 min/kb, then the temperature was held at 72° C. for 10 min and the end temperature was 4° C. The resulting fragments were sequence by sanger sequencing using the c9mfwdscreen forward primer and the c9mrevscreen reverse primer.

### Sequence Characterization for the Modified Nucleases “Zero”, “L1”, “L2” and “L3”.

A VHH domain was inserted without a linker and the resulting protein was labeled “Zero”. A VHH domain was inserted with the 16 amino acid linker L1 and the resulting protein was labeled “L1”. A VHH domain was inserted with the 23 amino acid linker L2 and the resulting protein was labeled “L2”. The complementarity-determining region (CDR) QQYYSYRT (SEQ ID NO: 63) was inserted and the resulting protein was labeled “L3”. The sequences for Zero, L1, L2 and L3 are presented in Table 9 below.

The sequences were generated by Gibson assembly/NEB hifi assembly, where C9m backbone was open at position ser1174 and homology arms generated by PCR upon a an anti 004 VHH encoded in DNA. Linkers were added at the same time by overhang PCR. Fragments were purified by silica spin columns and assembly by Gibson assembly with fragments denoting the Zero, L1 and L2 designs. Sequence confirmation of reassembly plasmid and insert showed that inserts were all in frame. Test gel filtration confirmed that the proteins can be purified and the tobacco etch virus (TEV) protease cleavage was functional.

TABLE 9
Sequences for Zero, L1, L2 and L3

| | | SEQ |
|---|---|---|
| | | ID |
| Name | Sequence | NO |
| Zero | ATGGATAAGAAATACTCAATAGGCTTAGATATCGGCACAAATAGCGTCGGATGGGCGGTG | 64 |
| (DNA) | ATCACTGATGAATATAAGGTTCCGTCTAAAAAGTTCAAGGTTCTGGGAAATACAGACCGC | |
| | CACAGTATCAAAAAAAATCTTATAGGGGCTCTTTTATTTGACAGTGGAGAGACAGCGGAA | |
| | GCGACTCGTCTCAAACGGACAGCTCGTAGAAGGTATACACGTCGGAAGAATCGTATTTGT | |
| | TATCTACAGGAGATTTTTTCAAATGAGATGGCGAAAGTAGATGATAGTTTCTTTCATCGA | |
| | CTTGAAGAGTCTTTTTTGGTGGAAGAAGACAAGAAGCATGAACGTCATCCTATTTTTGGA | |
| | AATATAGTAGATGAAGTTGCTTATCATGAGAAATATCCAACTATCTATCATCTGCGAAAA | |
| | AAATTGGTAGATTCTACTGATAAAGCGGATTTGCGCTTAATCTATTTGGCCTTAGCGCAT | |
| | ATGATTAAGTTTCGTGGTCATTTTTTGATTGAGGGAGATTTAAATCCTGATAATAGTGAT | |
| | GTGGACAAACTATTTATCCAGTTGGTACAAACCTACAATCAATTATTTGAAGAAAACCCT | |
| | ATTAACGCAAGTGGAGTAGATGCTAAAGCGATTCTTTCTGCACGATTGAGTAAATCAAGA | |
| | CGATTAGAAAATCTCATTGCTCAGCTCCCCGGTGAGAAGAAAAATGGCTTATTTGGGAAT | |
| | CTCATTGCTTTGTCATTGGGTTTGACCCCTAATTTTAAATCAAATTTTGATTTGGCAGAA | |
| | GATGCTAAATTACAGCTTTCAAAAGATACTTACGATGATGATTTAGATAATTTATTGGCG | |
| | CAAATTGGAGATCAATATGCTGATTTGTTTTTGGCAGCTAAGAATTTATCAGATGCTATT | |
| | TTACTTTCAGATATCCTAAGAGTAAATACTGAAATAACTAAGGCTCCCCTATCAGCTTCA | |
| | ATGATTAAACGCTACGATGAACATCATCAAGACTTGACTCTTTTAAAAGCTTTAGTTCGA | |
| | CAACAACTTCCAGAAAAGTATAAAGAAATCTTTTTTGATCAATCAAAAAACGGATATGCA | |
| | GGTTATATTGATGGGGGAGCTAGCCAAGAAGAATTTTATAAATTTATCAAACCAATTTTA | |
| | GAAAAAATGGATGGTACTGAGGAATTATTGGTGAAACTAAATCGTGAAGATTTGCTGCGC | |
| | AAGCAACGGACCTTTGACAACGGCTCTATTCCCCATCAAATTCACTTGGGTGAGCTGCAT | |
| | GCTATTTTGAGAAGACAAGAAGACTTTTATCCATTTTTAAAAGACAATCGTGAGAAGATT | |
| | GAAAAAATCTTGACTTTTCGAATTCCTTATTATGTTGGTCCATTGGCGCGTGGCAATAGT | |
| | CGTTTTGCATGGATGACTCGGAAGTCTGAAGAAACAATTACCCCATGGAATTTTGAAGAA | |
| | GTTGTCGATAAAGGTGCTTCAGCTCAATCATTTATTGAACGCATGACAAACTTTGATAAA | |
| | AATCTTCCAAATGAAAAAGTACTACCAAAACATAGTTTGCTTTATGAGTATTTTACGGTT | |
| | TATAACGAATTGACAAAGGTCAAATATGTTACTGAAGGAATGCGAAAACCAGCATTTCTT | |
| | TCAGGTGAACAGAAGAAAGCCATTGTTGATTTACTCTTCAAAACAAATCGAAAAGTAACC | |
| | GTTAAGCAATTAAAAGAAGATTATTTCAAAAAAATAGAATGTTTTGATAGTGTTGAAATT | |
| | TCAGGAGTTGAAGATAGATTTAATGCTTCATTAGGTACCTACCATGATTTGCTAAAAATT | |
| | ATTAAAGATAAAGATTTTTTGGATAATGAAGAAAATGAAGATATCTTAGAGGATATTGTT | |
| | TTAACATTGACCTTATTTGAAGATAGGGAGATGATTGAGGAAAGACTTAAAACATATGCT | |
| | CACCTCTTTGATGATAAGGTGATGAAACAGCTTAAACGTCGCCGTTATACTGGTTGGGGA | |
| | CGTTTGTCTCGAAAATTGATTAATGGTATTAGGGATAAGCAATCTGGCAAAACAATATTA | |
| | GATTTTTTGAAATCAGATGGTTTTGCCAATCGCAATTTTATGCAGCTGATCCATGATGAT | |
| | AGTTTGACATTTAAAGAAGACATTCAAAAAGCACAAGTGTCTGGACAAGGCGATAGTTTA | |
| | CATGAACATATTGCAAATTTAGCTGGTAGCCCTGCTATTAAAAAAGGTATTTTACAGACT | |
| | GTAAAAGTTGTTGATGAATTGGTCAAAGTAATGGGGCGGCATAAGCCAGAAAATATCGTT | |
| | ATTGAAATGGCACGTGAAAATCAGACAACTCAAAAGGGCCAGAAAAATTCGCGAGAGCGT | |
| | ATGAAACGAATCGAAGAAGGTATCAAAGAATTAGGAAGTCAGATTCTTAAAGAGCATCCT | |
| | GTTGAAAATACTCAATTGCAAAATGAAAAGCTCTATCTCTATTATCTCCAAAATGGAAGA | |
| | GACATGTATGTGGACCAAGAATTAGATATTAATCGTTTAAGTGATTATGATGTCGATCAC | |
| | ATTGTTCCACAAAGTTTCCTTAAAGACGATTCAATAGACAATAAGGTCTTAACGCGTTCT | |
| | GATAAAAATCGTGGTAAATCGGATAACGTTCCAAGTGAAGAAGTAGTCAAAAAGATGAAA | |
| | AACTATTGGAGACAACTTCTAAACGCCAAGTTAATCACTCAACGTAAGTTTGATAATTTA | |
| | ACGAAAGCTGAACGTGGAGGTTTGAGTGAACTTGATAAAGCTGGTTTTATCAAACGCCAA | |
| | TTGGTTGAAACTCGCCAAATCACTAAGCATGTGGCACAAATTTTGGATAGTCGCATGAAT | |
| | ACTAAATACGATGAAAATGATAAACTTATTCGAGAGGTTAAAGTGATTACCTTAAAATCT | |
| | AAATTAGTTTCTGACTTCCGAAAAGATTTCCAATTCTATAAAGTACGTGAGATTAACAAT | |
| | TACCATCATGCCCATGATGCGTATCTAAATGCCGTCGTTGGAACTGCTTTGATTAAGAAA | |
| | TATCCAAAACTTGAATCGGAGTTTGTCTATGGTGATTATAAAGTTTATGATGTTCGTAAA | |
| | ATGATTGCTAAGTCTGAGCAAGAAATAGGCAAAGCAACCGCAAAATATTTCTTTTACTCT | |
| | AATATCATGAACTTCTTCAAAACAGAAATTACACTTGCAAATGGAGAGATTCGCAAACGC | |
| | CCTCTAATCGAAACTAATGGGGAAACTGGAGAAATTGTCTGGGATAAAGGGCGAGATTTT | |
| | GCCACAGTGCGCAAAGTATTGTCCATGCCCCAAGTCAATATTGTCAAGAAAACAGAAGTA | |
| | CAGACAGGCGGATTCTCCAAGGAGTCAATTTTACCAAAAAGAAATTCGGACAAGCTTATT | |
| | GCTCGTAAAAAAGACTGGGATCCAAAAAAATATGGTGGTTTTGATAGTCCAACGGTAGCT | |
| | TATTCAGTCCTAGTGGTTGCTAAGGTGGAAAAAGGGAAATCGGATCGATGGGGATCCCAG | |
| | GTTCAGCTGGTTCAGAGCGGTGGTGGTCTGGTTCAGGCAGGCGGTAGCCTGCGTCTGAGC | |
| | TGTGCATTTAGCGGTCGTACCTTTAGCATGTATACCATGGGTTGGTTTCGTCAGGCACCG | |
| | GGTAAAGAACGTGAATTTGTTGCAGCAAATCGTGGTCGTGGTCTGAGTCCGGATATTGCA | |
| | GATAGCGTTAATGGTCGTTTTACCATTAGCCGTGATAATGCCAAAAATACCCTGTACCTG | |
| | CAGATGGATAGCCTGAAACCGGAAGATACCGCAGTGTATTATTGTGCAGCAGCAAGCCGT | |
| | GAAGATCCGCCTGGTTATTGGGGTCAGGGCACCACCGTTACCGTTAGCAGCAAGAAGTTA | |
| | AAATCCGTTAAAGAGTTACTAGGGATCACAATTATGGAAAGAAGTTCCTTTGAAAAAAAT | |
| | CCGATTGACTTTTTAGAAGCTAAAGGATATAAGGAAGTTAAAAAAGACTTAATCATTAAA | |
| | CTACCTAAATATAGTCTTTTTGAGTTAGAAAACGGTCGTAAACGGATGCTGGCTAGTGCC | |
| | GGAGAATTACAAAAAGGAAATGAGCTGGCTCTGCCAAGCAAATATGTGAATTTTTTATAT | |
| | TTAGCTAGTCATTATGAAAAGTTGAAGGGTAGTCCAGAAGATAACGAACAAAAACAATTG | |
| | TTTGTGGAGCAGCATAAGCATTATTTAGATGAGATTATTGAGCAAATCAGTGAATTTTCT | |
| | AAGCGTGTTATTTTAGCAGATGCCAATTTAGATAAAGTTCTTAGTGCATATAACAAACAT | |
| | AGAGACAAACCAATACGTGAACAAGCAGAAAATATTATTCATTTATTTACGTTGACGAAT | |
| | CTTGGAGCTCCCGCTGCTTTTAAATATTTTGATACAACAATTGATCGTAAACGATATACG | |
| | TCTACAAAAGAAGTTTTAGATGCCACTCTTATCCATCAATCCATCACTGGTCTTTATGAA | |
| | ACACGCATTGATTTGAGTCAGCTAGGAGGTGACGGGTCACCTAAGAAAAAACGAAAAGTT | |
| | GAGGATCCTAAAAAGAAACGAAAAGTTGATAGCGGTTCAGAGACCCCAGGAACTAGCGAG | |
| | AGCGCTACACCGGAATCGGCGGAAGCGGGTATCACCGGCACGTGGTACAACCAGTCTGGT | |
| | TCTACCTTCACCGTTACCGCGGGTGCGGACGGTAACCTGACCGGTCAGTACGAAAACCGT | |
| | GCGCAGGGCACTGGTTGCCAGAACTCTCCGTACACCCTGACCGGTCGTTACAACGGTACC | |
| | AAACTGGAATGGCGTGTTGAATGGAACAACTCTACCGAAAACTGCCACTCTCGTACCGAA | |
| | TGGCGTGGTCAGTACCAGGGTGGTGCGGAAGCGCGTATCAACACCCAGTGGAACCTGACC | |
| | TACGAAGGTGGTTCTGGTCCGGCGACCGAACAGGGTCAGGACACCTTCACCAAAGTTAAA | |
| | CCGTCTGCGGCGTCTTAA | |
| Zero | Underlined: Nuclease | 65 |
| (annotated) | Bold: maltose bindind protein (MBP) + TEV site | |
| | Internal VHH: Italics | |
| | NLS: Bold and italic | |
| | His-tag: bold and underlined. | |
| | Monoavidin: dot underline and bold | |
| | CACCATCACCATCACCATGGAAAAATCGAAGAAGGTAAACTGGTAATCTGGATTAACGGC | |
| | GATAAAGGCTATAACGGTCTCGCTGAAGTCGGTAAGAAATTCGAGAAAGATACCGGAATT | |
| | AAAGTCACCGTTGAGCATCCGGATAAACTGGAAGAGAAATTCCCACAGGTTGCGGCAACT | |
| | GGCGATGGCCCTGACATTATCTTCTGGGCACACGACCGCTTTGGTGGCTACGCTCAATCT | |
| | GGCCTGTTGGCTGAAATCACCCCGGACAAAGCGTTCCAGGACAAGCTGTATCCGTTTACC | |
| | TGGGATGCCGTACGTTACAACGGCAAGCTGATTGCTTACCCGATCGCTGTTGAAGCGTTA | |
| | TCGCTGATTTATAACAAAGATCTGCTGCCGAACCCGCCAAAAACCTGGGAAGAGATCCCG | |
| | GCGCTGGATAAAGAACTGAAAGCGAAAGGTAAGAGCGCGCTGATGTTCAACCTGCAAGAA | |
| | CCGTACTTCACCTGGCCGCTGATTGCTGCTGACGGGGGTTATGCGTTCAAGTATGAAAAC | |
| | GGCAAGTACGACATTAAAGACGTGGGCGTGGATAACGCTGGCGCGAAAGCGGGTCTGACC | |
| | TTCCTGGTTGACCTGATTAAAAACAAACACATGAATGCAGACACCGATTACTCCATCGCA | |
| | GAAGCTGCCTTTAATAAAGGCGAAACAGCGATGACCATCAACGGCCCGTGGGCATGGTCC | |
| | AACATCGACACCAGCAAAGTGAATTATGGTGTAACGGTACTGCCGACCTTCAAGGGTCAA | |
| | CCATCCAAACCGTTCGTTGGCGTGCTGAGCGCAGGTATTAACGCCGCCAGTCCGAACAAA | |
| | GAGCTGGCAAAAGAGTTCCTCGAAAACTATCTGCTGACTGATGAAGGTCTGGAAGCGGTT | |
| | AATAAAGACAAACCGCTGGGTGCCGTAGCGCTGAAGTCTTACGAGGAAGAGTTGGCGAAA | |
| | GATCCACGTATTGCCGCCACTATGGAAAACGCCCAGAAAGGTGAAATCATGCCGAACATC | |
| | CCGCAGATGTCCGCTTTCTGGTATGCCGTGCGTACTGCGGTGATCAACGCCGCCAGCGGT | |
| | CGTCAGACTGTCGATGAAGCCCTGAAAGACGCGCAGACTAATTCGAGCTCGAACAACAAC | |
| | AACACTAGTGAAAACCTGTATTTCCAGGGAGCAGCCTCGATGGATAAGAAATACTCAATA | |
| | GGCTTAGATATCGGCACAAATAGCGTCGGATGGGCGGTGATCACTGATGAATATAAGGTT | |
| | CCGTCTAAAAAGTTCAAGGTTCTGGGAAATACAGACCGCCACAGTATCAAAAAAAATCTT | |
| | ATAGGGGCTCTTTTATTTGACAGTGGAGAGACAGCGGAAGCGACTCGTCTCAAACGGACA | |
| | GCTCGTAGAAGGTATACACGTCGGAAGAATCGTATTTGTTATCTACAGGAGATTTTTTCA | |
| | AATGAGATGGCGAAAGTAGATGATAGTTTCTTTCATCGACTTGAAGAGTCTTTTTTGGTG | |
| | GAAGAAGACAAGAAGCATGAACGTCATCCTATTTTTGGAAATATAGTAGATGAAGTTGCT | |
| | TATCATGAGAAATATCCAACTATCTATCATCTGCGAAAAAAATTGGTAGATTCTACTGAT | |
| | AAAGCGGATTTGCGCTTAATCTATTTGGCCTTAGCGCATATGATTAAGTTTCGTGGTCAT | |
| | TTTTTGATTGAGGGAGATTTAAATCCTGATAATAGTGATGTGGACAAACTATTTATCCAG | |
| | TTGGTACAAACCTACAATCAATTATTTGAAGAAAACCCTATTAACGCAAGTGGAGTAGAT | |
| | GCTAAAGCGATTCTTTCTGCACGATTGAGTAAATCAAGACGATTAGAAAATCTCATTGCT | |
| | CAGCTCCCCGGTGAGAAGAAAAATGGCTTATTTGGGAATCTCATTGCTTTGTCATTGGGT | |
| | TTGACCCCTAATTTTAAATCAAATTTTGATTTGGCAGAAGATGCTAAATTACAGCTTTCA | |
| | AAAGATACTTACGATGATGATTTAGATAATTTATTGGCGCAAATTGGAGATCAATATGCT | |
| | GATTTGTTTTTGGCAGCTAAGAATTTATCAGATGCTATTTTACTTTCAGATATCCTAAGA | |
| | GTAAATACTGAAATAACTAAGGCTCCCCTATCAGCTTCAATGATTAAACGCTACGATGAA | |
| | CATCATCAAGACTTGACTCTTTTAAAAGCTTTAGTTCGACAACAACTTCCAGAAAAGTAT | |
| | AAAGAAATCTTTTTTGATCAATCAAAAAACGGATATGCAGGTTATATTGATGGGGGAGCT | |
| | AGCCAAGAAGAATTTTATAAATTTATCAAACCAATTTTAGAAAAAATGGATGGTACTGAG | |
| | GAATTATTGGTGAAACTAAATCGTGAAGATTTGCTGCGCAAGCAACGGACCTTTGACAAC | |
| | GGCTCTATTCCCCATCAAATTCACTTGGGTGAGCTGCATGCTATTTTGAGAAGACAAGAA | |
| | GACTTTTATCCATTTTTAAAAGACAATCGTGAGAAGATTGAAAAAATCTTGACTTTTCGA | |
| | ATTCCTTATTATGTTGGTCCATTGGCGCGTGGCAATAGTCGTTTTGCATGGATGACTCGG | |
| | AAGTCTGAAGAAACAATTACCCCATGGAATTTTGAAGAAGTTGTCGATAAAGGTGCTTCA | |
| | GCTCAATCATTTATTGAACGCATGACAAACTTTGATAAAAATCTTCCAAATGAAAAAGTA | |
| | CTACCAAAACATAGTTTGCTTTATGAGTATTTTACGGTTTATAACGAATTGACAAAGGTC | |
| | AAATATGTTACTGAAGGAATGCGAAAACCAGCATTTCTTTCAGGTGAACAGAAGAAAGCC | |
| | ATTGTTGATTTACTCTTCAAAACAAATCGAAAAGTAACCGTTAAGCAATTAAAAGAAGAT | |
| | TATTTCAAAAAAATAGAATGTTTTGATAGTGTTGAAATTTCAGGAGTTGAAGATAGATTT | |
| | AATGCTTCATTAGGTACCTACCATGATTTGCTAAAAATTATTAAAGATAAAGATTTTTTG | |
| | GATAATGAAGAAAATGAAGATATCTTAGAGGATATTGTTTTAACATTGACCTTATTTGAA | |
| | GATAGGGAGATGATTGAGGAAAGACTTAAAACATATGCTCACCTCTTTGATGATAAGGTG | |
| | ATGAAACAGCTTAAACGTCGCCGTTATACTGGTTGGGGACGTTTGTCTCGAAAATTGATT | |
| | AATGGTATTAGGGATAAGCAATCTGGCAAAACAATATTAGATTTTTTGAAATCAGATGGT | |
| | TTTGCCAATCGCAATTTTATGCAGCTGATCCATGATGATAGTTTGACATTTAAAGAAGAC | |
| | ATTCAAAAAGCACAAGTGTCTGGACAAGGCGATAGTTTACATGAACATATTGCAAATTTA | |
| | GCTGGTAGCCCTGCTATTAAAAAAGGTATTTTACAGACTGTAAAAGTTGTTGATGAATTG | |
| | GTCAAAGTAATGGGGCGGCATAAGCCAGAAAATATCGTTATTGAAATGGCACGTGAAAAT | |
| | CAGACAACTCAAAAGGGCCAGAAAAATTCGCGAGAGCGTATGAAACGAATCGAAGAAGGT | |
| | ATCAAAGAATTAGGAAGTCAGATTCTTAAAGAGCATCCTGTTGAAAATACTCAATTGCAA | |
| | AATGAAAAGCTCTATCTCTATTATCTCCAAAATGGAAGAGACATGTATGTGGACCAAGAA | |
| | TTAGATATTAATCGTTTAAGTGATTATGATGTCGATCACATTGTTCCACAAAGTTTCCTT | |
| | AAAGACGATTCAATAGACAATAAGGTCTTAACGCGTTCTGATAAAAATCGTGGTAAATCG | |
| | GATAACGTTCCAAGTGAAGAAGTAGTCAAAAAGATGAAAAACTATTGGAGACAACTTCTA | |
| | AACGCCAAGTTAATCACTCAACGTAAGTTTGATAATTTAACGAAAGCTGAACGTGGAGGT | |
| | TTGAGTGAACTTGATAAAGCTGGTTTTATCAAACGCCAATTGGTTGAAACTCGCCAAATC | |
| | ACTAAGCATGTGGCACAAATTTTGGATAGTCGCATGAATACTAAATACGATGAAAATGAT | |
| | AAACTTATTCGAGAGGTTAAAGTGATTACCTTAAAATCTAAATTAGTTTCTGACTTCCGA | |
| | AAAGATTTCCAATTCTATAAAGTACGTGAGATTAACAATTACCATCATGCCCATGATGCG | |
| | TATCTAAATGCCGTCGTTGGAACTGCTTTGATTAAGAAATATCCAAAACTTGAATCGGAG | |
| | TTTGTCTATGGTGATTATAAAGTTTATGATGTTCGTAAAATGATTGCTAAGTCTGAGCAA | |
| | GAAATAGGCAAAGCAACCGCAAAATATTTCTTTTACTCTAATATCATGAACTTCTTCAAA | |
| | ACAGAAATTACACTTGCAAATGGAGAGATTCGCAAACGCCCTCTAATCGAAACTAATGGG | |
| | GAAACTGGAGAAATTGTCTGGGATAAAGGGCGAGATTTTGCCACAGTGCGCAAAGTATTG | |
| | TCCATGCCCCAAGTCAATATTGTCAAGAAAACAGAAGTACAGACAGGCGGATTCTCCAAG | |
| | GAGTCAATTTTACCAAAAAGAAATTCGGACAAGCTTATTGCTCGTAAAAAAGACTGGGAT | |
| | CCAAAAAAATATGGTGGTTTTGATAGTCCAACGGTAGCTTATTCAGTCCTAGTGGTTGCT | |
| | AAGGTGGAAAAAGGGAAATCGGATCGATGGGGATCCCAGGTTCAGCTGGTTCAGAGCGGT | |
| | GGTGGTCTGGTTCAGGCAGGCGGTAGCCTGCGTCTGAGCTGTGCATTTAGCGGTCGTACC | |
| | TTTAGCATGTATACCATGGGTTGGTTTCGTCAGGCACCGGGTAAAGAACGTGAATTTGTT | |
| | GCAGCAAATCGTGGTCGTGGTCTGAGTCCGGATATTGCAGATAGCGTTAATGGTCGTTTT | |
| | ACCATTAGCCGTGATAATGCCAAAAATACCCTGTACCTGCAGATGGATAGCCTGAAACCG | |
| | GAAGATACCGCAGTGTATTATTGTGCAGCAGCAAGCCGTGAAGATCCGCCTGGTTATTGG | |
| | GGTCAGGGCACCACCGTTACCGTTAGCAGCAAGAAGTTAAAATCCGTTAAAGAGTTACTA | |
| | GGGATCACAATTATGGAAAGAAGTTCCTTTGAAAAAAATCCGATTGACTTTTTAGAAGCT | |
| | AAAGGATATAAGGAAGTTAAAAAAGACTTAATCATTAAACTACCTAAATATAGTCTTTTT | |
| | GAGTTAGAAAACGGTCGTAAACGGATGCTGGCTAGTGCCGGAGAATTACAAAAAGGAAAT | |
| | GAGCTGGCTCTGCCAAGCAAATATGTGAATTTTTTATATTTAGCTAGTCATTATGAAAAG | |
| | TTGAAGGGTAGTCCAGAAGATAACGAACAAAAACAATTGTTTGTGGAGCAGCATAAGCAT | |
| | TATTTAGATGAGATTATTGAGCAAATCAGTGAATTTTCTAAGCGTGTTATTTTAGCAGAT | |
| | GCCAATTTAGATAAAGTTCTTAGTGCATATAACAAACATAGAGACAAACCAATACGTGAA | |
| | CAAGCAGAAAATATTATTCATTTATTTACGTTGACGAATCTTGGAGCTCCCGCTGCTTTT | |
| | AAATATTTTGATACAACAATTGATCGTAAACGATATACGTCTACAAAAGAAGTTTTAGAT | |
| | GCCACTCTTATCCATCAATCCATCACTGGTCTTTATGAAACACGCATTGATTTGAGTCAG | |
| | CTAGGAGGTGACGGGTCACCTAAGAAAAAACGAAAAGTTGAGGATCCTAAAAAGAAACGA | |
| | 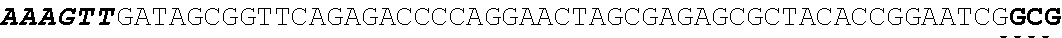 | |
| | 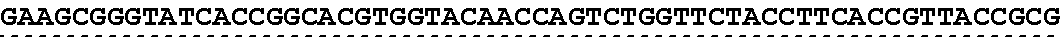 | |
| | 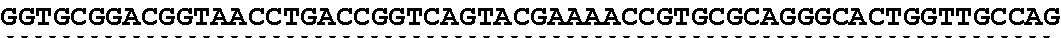 | |
| | 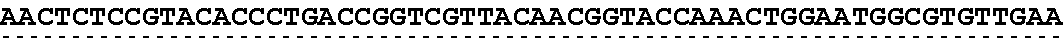 | |
| | 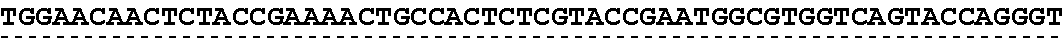 | |
| | 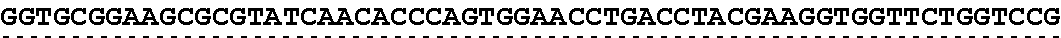 | |
| | 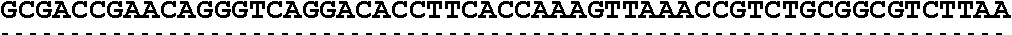 | |
| Zero | MDKKYSIGLDIGTNSVGWAVITDEYKVPSKKFKVLGNTDRHSIKKNLIGALLFDSGETAE | 66 |
| (a.a.) | ATRLKRTARRRYTRRKNRICYLQEIFSNEMAKVDDSFFHRLEESFLVEEDKKHERHPIFG | |
| | NIVDEVAYHEKYPTIYHLRKKLVDSTDKADLRLIYLALAHMIKERGHFLIEGDLNPDNSD | |
| | VDKLFIQLVQTYNQLFEENPINASGVDAKAILSARLSKSRRLENLIAQLPGEKKNGLFGN | |
| | LIALSLGLTPNFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEIFFDQSKNGYA | |
| | GYIDGGASQEEFYKFIKPILEKMDGTEELLVKLNREDLLRKORTFDNGSIPHQIHLGELH | |
| | AILRRQEDFYPFLKDNREKIEKILTFRIPYYVGPLARGNSRFAWMTRKSEETITPWNFEE | |
| | VVDKGASAQSFIERMTNFDKNLPNEKVLPKHSLLYEYFTVYNELTKVKYVTEGMRKPAFL | |
| | SGEQKKAIVDLLFKTNRKVTVKOLKEDYFKKIECFDSVEISGVEDRFNASLGTYHDLLKI | |
| | IKDKDFLDNEENEDILEDIVLTLTLFEDREMIEERLKTYAHLFDDKVMKQLKRRRYTGWG | |
| | RLSRKLINGIRDKQSGKTILDFLKSDGFANRNFMQLIHDDSLTFKEDIQKAQVSGQGDSL | |
| | HEHIANLAGSPAIKKGILQTVKVVDELVKVMGRHKPENIVIEMARENOTTQKGQKNSRER | |
| | MKRIEEGIKELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWROLLNAKLITORKFDNL | |
| | TKAERGGLSELDKAGFIKRQLVETROITKHVAQILDSRMNTKYDENDKLIREVKVITLKS | |
| | KLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDF | |
| | ATVRKVLSMPQVNIVKKTEVQTGGFSKESILPKRNSDKLIARKKDWDPKKYGGFDSPTVA | |
| | YSVLVVAKVEKGKSDRWGSQVQLVQSGGGLVQAGGSLRLSCAFSGRTFSMYTMGWFRQAP | |
| | GKEREFVAANRGRGLSPDIADSVNGRFTISRDNAKNTLYLQMDSLKPEDTAVYYCAAASR | |
| | EDPPGYWGQGTTVTVSSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQKQL | |
| | FVEQHKHYLDEIIEQISEFSKRVILADANLDKVLSAYNKHRDKPIREQAENIIHLFTLTN | |
| | LGAPAAFKYFDTTIDRKRYTSTKEVLDATLIHQSITGLYETRIDLSQLGGDGSPKKKRKV | |
| | EDPKKKRKVDSGSETPGTSESATPESAEAGITGTWYNQSGSTFTVTAGADGNLTGQYENR | |
| | AQGTGCONSPYTLTGRYNGTKLEWRVEWNNSTENCHSRTEWRGQYQGGAEARINTOWNLT | |
| | YEGGSGPATEQGQDTFTKVKPSAAS | |
| L1 (DNA) | CACCATCACCATCACCATGGAAAAATCGAAGAAGGTAAACTGGTAATCTGGATTAACGGC | 67 |
| | GATAAAGGCTATAACGGTCTCGCTGAAGTCGGTAAGAAATTCGAGAAAGATACCGGAATT | |
| | AAAGTCACCGTTGAGCATCCGGATAAACTGGAAGAGAAATTCCCACAGGTTGCGGCAACT | |
| | GGCGATGGCCCTGACATTATCTTCTGGGCACACGACCGCTTTGGTGGCTACGCTCAATCT | |
| | GGCCTGTTGGCTGAAATCACCCCGGACAAAGCGTTCCAGGACAAGCTGTATCCGTTTACC | |
| | TGGGATGCCGTACGTTACAACGGCAAGCTGATTGCTTACCCGATCGCTGTTGAAGCGTTA | |
| | TCGCTGATTTATAACAAAGATCTGCTGCCGAACCCGCCAAAAACCTGGGAAGAGATCCCG | |
| | GCGCTGGATAAAGAACTGAAAGCGAAAGGTAAGAGCGCGCTGATGTTCAACCTGCAAGAA | |
| | CCGTACTTCACCTGGCCGCTGATTGCTGCTGACGGGGGTTATGCGTTCAAGTATGAAAAC | |
| | GGCAAGTACGACATTAAAGACGTGGGCGTGGATAACGCTGGCGCGAAAGCGGGTCTGACC | |
| | TTCCTGGTTGACCTGATTAAAAACAAACACATGAATGCAGACACCGATTACTCCATCGCA | |
| | GAAGCTGCCTTTAATAAAGGCGAAACAGCGATGACCATCAACGGCCCGTGGGCATGGTCC | |
| | AACATCGACACCAGCAAAGTGAATTATGGTGTAACGGTACTGCCGACCTTCAAGGGTCAA | |
| | CCATCCAAACCGTTCGTTGGCGTGCTGAGCGCAGGTATTAACGCCGCCAGTCCGAACAAA | |
| | GAGCTGGCAAAAGAGTTCCTCGAAAACTATCTGCTGACTGATGAAGGTCTGGAAGCGGTT | |
| | AATAAAGACAAACCGCTGGGTGCCGTAGCGCTGAAGTCTTACGAGGAAGAGTTGGCGAAA | |
| | GATCCACGTATTGCCGCCACTATGGAAAACGCCCAGAAAGGTGAAATCATGCCGAACATC | |
| | CCGCAGATGTCCGCTTTCTGGTATGCCGTGCGTACTGCGGTGATCAACGCCGCCAGCGGT | |
| | CGTCAGACTGTCGATGAAGCCCTGAAAGACGCGCAGACTAATTCGAGCTCGAACAACAAC | |
| | AACACTAGTGAAAACCTGTATTTCCAGGGAGCAGCCTCGATGGATAAGAAATACTCAATA | |
| | GGCTTAGATATCGGCACAAATAGCGTCGGATGGGCGGTGATCACTGATGAATATAAGGTT | |
| | CCGTCTAAAAAGTTCAAGGTTCTGGGAAATACAGACCGCCACAGTATCAAAAAAAATCTT | |
| | ATAGGGGCTCTTTTATTTGACAGTGGAGAGACAGCGGAAGCGACTCGTCTCAAACGGACA | |
| | GCTCGTAGAAGGTATACACGTCGGAAGAATCGTATTTGTTATCTACAGGAGATTTTTTCA | |
| | AATGAGATGGCGAAAGTAGATGATAGTTTCTTTCATCGACTTGAAGAGTCTTTTTTGGTG | |
| | GAAGAAGACAAGAAGCATGAACGTCATCCTATTTTTGGAAATATAGTAGATGAAGTTGCT | |
| | TATCATGAGAAATATCCAACTATCTATCATCTGCGAAAAAAATTGGTAGATTCTACTGAT | |
| | AAAGCGGATTTGCGCTTAATCTATTTGGCCTTAGCGCATATGATTAAGTTTCGTGGTCAT | |
| | TTTTTGATTGAGGGAGATTTAAATCCTGATAATAGTGATGTGGACAAACTATTTATCCAG | |
| | TTGGTACAAACCTACAATCAATTATTTGAAGAAAACCCTATTAACGCAAGTGGAGTAGAT | |
| | GCTAAAGCGATTCTTTCTGCACGATTGAGTAAATCAAGACGATTAGAAAATCTCATTGCT | |
| | CAGCTCCCCGGTGAGAAGAAAAATGGCTTATTTGGGAATCTCATTGCTTTGTCATTGGGT | |
| | TTGACCCCTAATTTTAAATCAAATTTTGATTTGGCAGAAGATGCTAAATTACAGCTTTCA | |
| | AAAGATACTTACGATGATGATTTAGATAATTTATTGGCGCAAATTGGAGATCAATATGCT | |
| | GATTTGTTTTTGGCAGCTAAGAATTTATCAGATGCTATTTTACTTTCAGATATCCTAAGA | |
| | GTAAATACTGAAATAACTAAGGCTCCCCTATCAGCTTCAATGATTAAACGCTACGATGAA | |
| | CATCATCAAGACTTGACTCTTTTAAAAGCTTTAGTTCGACAACAACTTCCAGAAAAGTAT | |
| | AAAGAAATCTTTTTTGATCAATCAAAAAACGGATATGCAGGTTATATTGATGGGGGAGCT | |
| | AGCCAAGAAGAATTTTATAAATTTATCAAACCAATTTTAGAAAAAATGGATGGTACTGAG | |
| | GAATTATTGGTGAAACTAAATCGTGAAGATTTGCTGCGCAAGCAACGGACCTTTGACAAC | |
| | GGCTCTATTCCCCATCAAATTCACTTGGGTGAGCTGCATGCTATTTTGAGAAGACAAGAA | |
| | GACTTTTATCCATTTTTAAAAGACAATCGTGAGAAGATTGAAAAAATCTTGACTTTTCGA | |
| | ATTCCTTATTATGTTGGTCCATTGGCGCGTGGCAATAGTCGTTTTGCATGGATGACTCGG | |
| | AAGTCTGAAGAAACAATTACCCCATGGAATTTTGAAGAAGTTGTCGATAAAGGTGCTTCA | |
| | GCTCAATCATTTATTGAACGCATGACAAACTTTGATAAAAATCTTCCAAATGAAAAAGTA | |
| | CTACCAAAACATAGTTTGCTTTATGAGTATTTTACGGTTTATAACGAATTGACAAAGGTC | |
| | AAATATGTTACTGAAGGAATGCGAAAACCAGCATTTCTTTCAGGTGAACAGAAGAAAGCC | |
| | ATTGTTGATTTACTCTTCAAAACAAATCGAAAAGTAACCGTTAAGCAATTAAAAGAAGAT | |
| | TATTTCAAAAAAATAGAATGTTTTGATAGTGTTGAAATTTCAGGAGTTGAAGATAGATTT | |
| | AATGCTTCATTAGGTACCTACCATGATTTGCTAAAAATTATTAAAGATAAAGATTTTTTG | |
| | GATAATGAAGAAAATGAAGATATCTTAGAGGATATTGTTTTAACATTGACCTTATTTGAA | |
| | GATAGGGAGATGATTGAGGAAAGACTTAAAACATATGCTCACCTCTTTGATGATAAGGTG | |
| | ATGAAACAGCTTAAACGTCGCCGTTATACTGGTTGGGGACGTTTGTCTCGAAAATTGATT | |
| | AATGGTATTAGGGATAAGCAATCTGGCAAAACAATATTAGATTTTTTGAAATCAGATGGT | |
| | TTTGCCAATCGCAATTTTATGCAGCTGATCCATGATGATAGTTTGACATTTAAAGAAGAC | |
| | ATTCAAAAAGCACAAGTGTCTGGACAAGGCGATAGTTTACATGAACATATTGCAAATTTA | |
| | GCTGGTAGCCCTGCTATTAAAAAAGGTATTTTACAGACTGTAAAAGTTGTTGATGAATTG | |
| | GTCAAAGTAATGGGGCGGCATAAGCCAGAAAATATCGTTATTGAAATGGCACGTGAAAAT | |
| | CAGACAACTCAAAAGGGCCAGAAAAATTCGCGAGAGCGTATGAAACGAATCGAAGAAGGT | |
| | ATCAAAGAATTAGGAAGTCAGATTCTTAAAGAGCATCCTGTTGAAAATACTCAATTGCAA | |
| | AATGAAAAGCTCTATCTCTATTATCTCCAAAATGGAAGAGACATGTATGTGGACCAAGAA | |
| | TTAGATATTAATCGTTTAAGTGATTATGATGTCGATCACATTGTTCCACAAAGTTTCCTT | |
| | AAAGACGATTCAATAGACAATAAGGTCTTAACGCGTTCTGATAAAAATCGTGGTAAATCG | |
| | GATAACGTTCCAAGTGAAGAAGTAGTCAAAAAGATGAAAAACTATTGGAGACAACTTCTA | |
| | AACGCCAAGTTAATCACTCAACGTAAGTTTGATAATTTAACGAAAGCTGAACGTGGAGGT | |
| | TTGAGTGAACTTGATAAAGCTGGTTTTATCAAACGCCAATTGGTTGAAACTCGCCAAATC | |
| | ACTAAGCATGTGGCACAAATTTTGGATAGTCGCATGAATACTAAATACGATGAAAATGAT | |
| | AAACTTATTCGAGAGGTTAAAGTGATTACCTTAAAATCTAAATTAGTTTCTGACTTCCGA | |
| | AAAGATTTCCAATTCTATAAAGTACGTGAGATTAACAATTACCATCATGCCCATGATGCG | |
| | TATCTAAATGCCGTCGTTGGAACTGCTTTGATTAAGAAATATCCAAAACTTGAATCGGAG | |
| | TTTGTCTATGGTGATTATAAAGTTTATGATGTTCGTAAAATGATTGCTAAGTCTGAGCAA | |
| | GAAATAGGCAAAGCAACCGCAAAATATTTCTTTTACTCTAATATCATGAACTTCTTCAAA | |
| | ACAGAAATTACACTTGCAAATGGAGAGATTCGCAAACGCCCTCTAATCGAAACTAATGGG | |
| | GAAACTGGAGAAATTGTCTGGGATAAAGGGCGAGATTTTGCCACAGTGCGCAAAGTATTG | |
| | TCCATGCCCCAAGTCAATATTGTCAAGAAAACAGAAGTACAGACAGGCGGATTCTCCAAG | |
| | GAGTCAATTTTACCAAAAAGAAATTCGGACAAGCTTATTGCTCGTAAAAAAGACTGGGAT | |
| | CCAAAAAAATATGGTGGTTTTGATAGTCCAACGGTAGCTTATTCAGTCCTAGTGGTTGCT | |
| | AAGGTGGAAAAAGGGAAATCGGGGAGCGCAGGATCCGCTGCCGGTTCAGGAGAGTTTGAT | |
| | CGATGGGGATCCCAGGTTCAGCTGGTTCAGAGCGGTGGTGGTCTGGTTCAGGCAGGCGGT | |
| | AGCCTGCGTCTGAGCTGTGCATTTAGCGGTCGTACCTTTAGCATGTATACCATGGGTTGG | |
| | TTTCGTCAGGCACCGGGTAAAGAACGTGAATTTGTTGCAGCAAATCGTGGTCGTGGTCTG | |
| | AGTCCGGATATTGCAGATAGCGTTAATGGTCGTTTTACCATTAGCCGTGATAATGCCAAA | |
| | AATACCCTGTACCTGCAGATGGATAGCCTGAAACCGGAAGATACCGCAGTGTATTATTGT | |
| | GCAGCAGCAAGCCGTGAAGATCCGCCTGGTTATTGGGGTCAGGGCACCACCGTTACCGTT | |
| | AGCAGCAAGAAGTTAAAATCCGTTAAAGAGTTACTAGGGATCACAATTATGGAAAGAAGT | |
| | TCCTTTGAAAAAAATCCGATTGACTTTTTAGAAGCTAAAGGATATAAGGAAGTTAAAAAA | |
| | GACTTAATCATTAAACTACCTAAATATAGTCTTTTTGAGTTAGAAAACGGTCGTAAACGG | |
| | ATGCTGGCTAGTGCCGGAGAATTACAAAAAGGAAATGAGCTGGCTCTGCCAAGCAAATAT | |
| | GTGAATTTTTTATATTTAGCTAGTCATTATGAAAAGTTGAAGGGTAGTCCAGAAGATAAC | |
| | GAACAAAAACAATTGTTTGTGGAGCAGCATAAGCATTATTTAGATGAGATTATTGAGCAA | |
| | ATCAGTGAATTTTCTAAGCGTGTTATTTTAGCAGATGCCAATTTAGATAAAGTTCTTAGT | |
| | GCATATAACAAACATAGAGACAAACCAATACGTGAACAAGCAGAAAATATTATTCATTTA | |
| | TTTACGTTGACGAATCTTGGAGCTCCCGCTGCTTTTAAATATTTTGATACAACAATTGAT | |
| | CGTAAACGATATACGTCTACAAAAGAAGTTTTAGATGCCACTCTTATCCATCAATCCATC | |
| | ACTGGTCTTTATGAAACACGCATTGATTTGAGTCAGCTAGGAGGTGACGGGTCACCTAAG | |
| | AAAAAACGAAAAGTTGAGGATCCTAAAAAGAAACGAAAAGTTGATAGCGGTTCAGAGACC | |
| | CCAGGAACTAGCGAGAGCGCTACACCGGAATCGGCGGAAGCGGGTATCACCGGCACGTGG | |
| | TACAACCAGTCTGGTTCTACCTTCACCGTTACCGCGGGTGCGGACGGTAACCTGACCGGT | |
| | CAGTACGAAAACCGTGCGCAGGGCACTGGTTGCCAGAACTCTCCGTACACCCTGACCGGT | |
| | CGTTACAACGGTACCAAACTGGAATGGCGTGTTGAATGGAACAACTCTACCGAAAACTGC | |
| | CACTCTCGTACCGAATGGCGTGGTCAGTACCAGGGTGGTGCGGAAGCGCGTATCAACACC | |
| | CAGTGGAACCTGACCTACGAAGGTGGTTCTGGTCCGGCGACCGAACAGGGTCAGGACACC | |
| | TTCACCAAAGTTAAACCGTCTGCGGCGTCTTAA | |
| L1 | Underlined: Nuclease | 68 |
| (annotated) | Bold: MBP + TEV site | |
| | Internal VHH: Italics | |
| | NLS: Bold and italic | |
| | His-tag: bold and underlined. | |
| | Linker: dot underline | |
| | Monoavidin: dot underline and bold | |
| | CACCATCACCATCACCATGGAAAAATCGAAGAAGGTAAACTGGTAATCTGGATTAACGGC | |
| | GATAAAGGCTATAACGGTCTCGCTGAAGTCGGTAAGAAATTCGAGAAAGATACCGGAATT | |
| | AAAGTCACCGTTGAGCATCCGGATAAACTGGAAGAGAAATTCCCACAGGTTGCGGCAACT | |
| | GGCGATGGCCCTGACATTATCTTCTGGGCACACGACCGCTTTGGTGGCTACGCTCAATCT | |
| | GGCCTGTTGGCTGAAATCACCCCGGACAAAGCGTTCCAGGACAAGCTGTATCCGTTTACC | |
| | TGGGATGCCGTACGTTACAACGGCAAGCTGATTGCTTACCCGATCGCTGTTGAAGCGTTA | |
| | TCGCTGATTTATAACAAAGATCTGCTGCCGAACCCGCCAAAAACCTGGGAAGAGATCCCG | |
| | GCGCTGGATAAAGAACTGAAAGCGAAAGGTAAGAGCGCGCTGATGTTCAACCTGCAAGAA | |
| | CCGTACTTCACCTGGCCGCTGATTGCTGCTGACGGGGGTTATGCGTTCAAGTATGAAAAC | |
| | GGCAAGTACGACATTAAAGACGTGGGCGTGGATAACGCTGGCGCGAAAGCGGGTCTGACC | |
| | TTCCTGGTTGACCTGATTAAAAACAAACACATGAATGCAGACACCGATTACTCCATCGCA | |
| | GAAGCTGCCTTTAATAAAGGCGAAACAGCGATGACCATCAACGGCCCGTGGGCATGGTCC | |
| | AACATCGACACCAGCAAAGTGAATTATGGTGTAACGGTACTGCCGACCTTCAAGGGTCAA | |
| | CCATCCAAACCGTTCGTTGGCGTGCTGAGCGCAGGTATTAACGCCGCCAGTCCGAACAAA | |
| | GAGCTGGCAAAAGAGTTCCTCGAAAACTATCTGCTGACTGATGAAGGTCTGGAAGCGGTT | |
| | AATAAAGACAAACCGCTGGGTGCCGTAGCGCTGAAGTCTTACGAGGAAGAGTTGGCGAAA | |
| | GATCCACGTATTGCCGCCACTATGGAAAACGCCCAGAAAGGTGAAATCATGCCGAACATC | |
| | CCGCAGATGTCCGCTTTCTGGTATGCCGTGCGTACTGCGGTGATCAACGCCGCCAGCGGT | |
| | CGTCAGACTGTCGATGAAGCCCTGAAAGACGCGCAGACTAATTCGAGCTCGAACAACAAC | |
| | AACACTAGTGAAAACCTGTATTTCCAGGGAGCAGCCTCGATGGATAAGAAATACTCAATA | |
| | GGCTTAGATATCGGCACAAATAGCGTCGGATGGGCGGTGATCACTGATGAATATAAGGTT | |
| | CCGTCTAAAAAGTTCAAGGTTCTGGGAAATACAGACCGCCACAGTATCAAAAAAAATCTT | |
| | ATAGGGGCTCTTTTATTTGACAGTGGAGAGACAGCGGAAGCGACTCGTCTCAAACGGACA | |
| | GCTCGTAGAAGGTATACACGTCGGAAGAATCGTATTTGTTATCTACAGGAGATTTTTTCA | |
| | AATGAGATGGCGAAAGTAGATGATAGTTTCTTTCATCGACTTGAAGAGTCTTTTTTGGTG | |
| | GAAGAAGACAAGAAGCATGAACGTCATCCTATTTTTGGAAATATAGTAGATGAAGTTGCT | |
| | TATCATGAGAAATATCCAACTATCTATCATCTGCGAAAAAAATTGGTAGATTCTACTGAT | |
| | AAAGCGGATTTGCGCTTAATCTATTTGGCCTTAGCGCATATGATTAAGTTTCGTGGTCAT | |
| | TTTTTGATTGAGGGAGATTTAAATCCTGATAATAGTGATGTGGACAAACTATTTATCCAG | |
| | TTGGTACAAACCTACAATCAATTATTTGAAGAAAACCCTATTAACGCAAGTGGAGTAGAT | |
| | GCTAAAGCGATTCTTTCTGCACGATTGAGTAAATCAAGACGATTAGAAAATCTCATTGCT | |
| | CAGCTCCCCGGTGAGAAGAAAAATGGCTTATTTGGGAATCTCATTGCTTTGTCATTGGGT | |
| | TTGACCCCTAATTTTAAATCAAATTTTGATTTGGCAGAAGATGCTAAATTACAGCTTTCA | |
| | AAAGATACTTACGATGATGATTTAGATAATTTATTGGCGCAAATTGGAGATCAATATGCT | |
| | GATTTGTTTTTGGCAGCTAAGAATTTATCAGATGCTATTTTACTTTCAGATATCCTAAGA | |
| | GTAAATACTGAAATAACTAAGGCTCCCCTATCAGCTTCAATGATTAAACGCTACGATGAA | |
| | CATCATCAAGACTTGACTCTTTTAAAAGCTTTAGTTCGACAACAACTTCCAGAAAAGTAT | |
| | AAAGAAATCTTTTTTGATCAATCAAAAAACGGATATGCAGGTTATATTGATGGGGGAGCT | |
| | AGCCAAGAAGAATTTTATAAATTTATCAAACCAATTTTAGAAAAAATGGATGGTACTGAG | |
| | GAATTATTGGTGAAACTAAATCGTGAAGATTTGCTGCGCAAGCAACGGACCTTTGACAAC | |
| | GGCTCTATTCCCCATCAAATTCACTTGGGTGAGCTGCATGCTATTTTGAGAAGACAAGAA | |
| | GACTTTTATCCATTTTTAAAAGACAATCGTGAGAAGATTGAAAAAATCTTGACTTTTCGA | |
| | ATTCCTTATTATGTTGGTCCATTGGCGCGTGGCAATAGTCGTTTTGCATGGATGACTCGG | |
| | AAGTCTGAAGAAACAATTACCCCATGGAATTTTGAAGAAGTTGTCGATAAAGGTGCTTCA | |
| | GCTCAATCATTTATTGAACGCATGACAAACTTTGATAAAAATCTTCCAAATGAAAAAGTA | |
| | CTACCAAAACATAGTTTGCTTTATGAGTATTTTACGGTTTATAACGAATTGACAAAGGTC | |
| | AAATATGTTACTGAAGGAATGCGAAAACCAGCATTTCTTTCAGGTGAACAGAAGAAAGCC | |
| | ATTGTTGATTTACTCTTCAAAACAAATCGAAAAGTAACCGTTAAGCAATTAAAAGAAGAT | |
| | TATTTCAAAAAAATAGAATGTTTTGATAGTGTTGAAATTTCAGGAGTTGAAGATAGATTT | |
| | AATGCTTCATTAGGTACCTACCATGATTTGCTAAAAATTATTAAAGATAAAGATTTTTTG | |
| | GATAATGAAGAAAATGAAGATATCTTAGAGGATATTGTTTTAACATTGACCTTATTTGAA | |
| | GATAGGGAGATGATTGAGGAAAGACTTAAAACATATGCTCACCTCTTTGATGATAAGGTG | |
| | ATGAAACAGCTTAAACGTCGCCGTTATACTGGTTGGGGACGTTTGTCTCGAAAATTGATT | |
| | AATGGTATTAGGGATAAGCAATCTGGCAAAACAATATTAGATTTTTTGAAATCAGATGGT | |
| | TTTGCCAATCGCAATTTTATGCAGCTGATCCATGATGATAGTTTGACATTTAAAGAAGAC | |
| | ATTCAAAAAGCACAAGTGTCTGGACAAGGCGATAGTTTACATGAACATATTGCAAATTTA | |
| | GCTGGTAGCCCTGCTATTAAAAAAGGTATTTTACAGACTGTAAAAGTTGTTGATGAATTG | |
| | GTCAAAGTAATGGGGCGGCATAAGCCAGAAAATATCGTTATTGAAATGGCACGTGAAAAT | |
| | CAGACAACTCAAAAGGGCCAGAAAAATTCGCGAGAGCGTATGAAACGAATCGAAGAAGGT | |
| | ATCAAAGAATTAGGAAGTCAGATTCTTAAAGAGCATCCTGTTGAAAATACTCAATTGCAA | |
| | AATGAAAAGCTCTATCTCTATTATCTCCAAAATGGAAGAGACATGTATGTGGACCAAGAA | |
| | TTAGATATTAATCGTTTAAGTGATTATGATGTCGATCACATTGTTCCACAAAGTTTCCTT | |
| | AAAGACGATTCAATAGACAATAAGGTCTTAACGCGTTCTGATAAAAATCGTGGTAAATCG | |
| | GATAACGTTCCAAGTGAAGAAGTAGTCAAAAAGATGAAAAACTATTGGAGACAACTTCTA | |
| | AACGCCAAGTTAATCACTCAACGTAAGTTTGATAATTTAACGAAAGCTGAACGTGGAGGT | |
| | TTGAGTGAACTTGATAAAGCTGGTTTTATCAAACGCCAATTGGTTGAAACTCGCCAAATC | |
| | ACTAAGCATGTGGCACAAATTTTGGATAGTCGCATGAATACTAAATACGATGAAAATGAT | |
| | AAACTTATTCGAGAGGTTAAAGTGATTACCTTAAAATCTAAATTAGTTTCTGACTTCCGA | |
| | AAAGATTTCCAATTCTATAAAGTACGTGAGATTAACAATTACCATCATGCCCATGATGCG | |
| | TATCTAAATGCCGTCGTTGGAACTGCTTTGATTAAGAAATATCCAAAACTTGAATCGGAG | |
| | TTTGTCTATGGTGATTATAAAGTTTATGATGTTCGTAAAATGATTGCTAAGTCTGAGCAA | |
| | GAAATAGGCAAAGCAACCGCAAAATATTTCTTTTACTCTAATATCATGAACTTCTTCAAA | |
| | ACAGAAATTACACTTGCAAATGGAGAGATTCGCAAACGCCCTCTAATCGAAACTAATGGG | |
| | GAAACTGGAGAAATTGTCTGGGATAAAGGGCGAGATTTTGCCACAGTGCGCAAAGTATTG | |
| | TCCATGCCCCAAGTCAATATTGTCAAGAAAACAGAAGTACAGACAGGCGGATTCTCCAAG | |
| | GAGTCAATTTTACCAAAAAGAAATTCGGACAAGCTTATTGCTCGTAAAAAAGACTGGGAT | |
| | CCAAAAAAATATGGTGGTTTTGATAGTCCAACGGTAGCTTATTCAGTCCTAGTGGTTGCT | |
| | 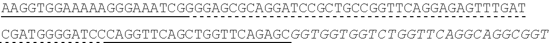 | |
| | AGCCTGCGTCTGAGCTGTGCATTTAGCGGTCGTACCTTTAGCATGTATACCATGGGTTGG | |
| | TTTCGTCAGGCACCGGGTAAAGAACGTGAATTTGTTGCAGCAAATCGTGGTCGTGGTCTG | |
| | AGTCCGGATATTGCAGATAGCGTTAATGGTCGTTTTACCATTAGCCGTGATAATGCCAAA | |
| | AATACCCTGTACCTGCAGATGGATAGCCTGAAACCGGAAGATACCGCAGTGTATTATTGT | |
| | GCAGCAGCAAGCCGTGAAGATCCGCCTGGTTATTGGGGTCAGGGCACCACCGTTACCGTT | |
| | AGCAGCAAGAAGTTAAAATCCGTTAAAGAGTTACTAGGGATCACAATTATGGAAAGAAGT | |
| | TCCTTTGAAAAAAATCCGATTGACTTTTTAGAAGCTAAAGGATATAAGGAAGTTAAAAAA | |
| | GACTTAATCATTAAACTACCTAAATATAGTCTTTTTGAGTTAGAAAACGGTCGTAAACGG | |
| | ATGCTGGCTAGTGCCGGAGAATTACAAAAAGGAAATGAGCTGGCTCTGCCAAGCAAATAT | |
| | GTGAATTTTTTATATTTAGCTAGTCATTATGAAAAGTTGAAGGGTAGTCCAGAAGATAAC | |
| | GAACAAAAACAATTGTTTGTGGAGCAGCATAAGCATTATTTAGATGAGATTATTGAGCAA | |
| | ATCAGTGAATTTTCTAAGCGTGTTATTTTAGCAGATGCCAATTTAGATAAAGTTCTTAGT | |
| | GCATATAACAAACATAGAGACAAACCAATACGTGAACAAGCAGAAAATATTATTCATTTA | |
| | TTTACGTTGACGAATCTTGGAGCTCCCGCTGCTTTTAAATATTTTGATACAACAATTGAT | |
| | CGTAAACGATATACGTCTACAAAAGAAGTTTTAGATGCCACTCTTATCCATCAATCCATC | |
| | ACTGGTCTTTATGAAACACGCATTGATTTGAGTCAGCTAGGAGGTGACGGGTCACCTAAG | |
| | AAAAAACGAAAAGTTGAGGATCCTAAAAAGAAACGAAAAGTTGATAGCGGTTCAGAGACC | |
| | 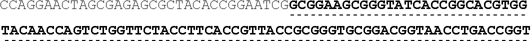 | |
| | 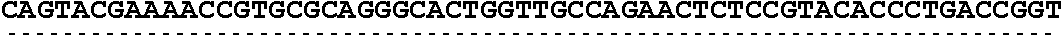 | |
| | 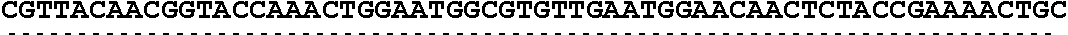 | |
| | 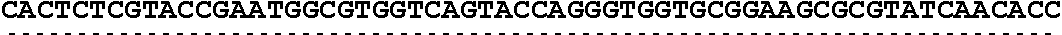 | |
| | 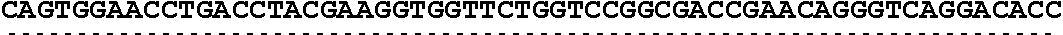 | |
| | 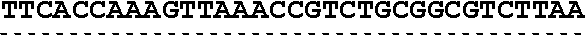 | |
| L1 (a.a.) | MDKKYSIGLDIGTNSVGWAVITDEYKVPSKKFKVLGNTDRHSIKKNLIGALLFDSGETAE | 69 |
| | ATRLKRTARRRYTRRKNRICYLQEIFSNEMAKVDDSFFHRLEESFLVEEDKKHERHPIFG | |
| | NIVDEVAYHEKYPTIYHLRKKLVDSTDKADLRLIYLALAHMIKFRGHFLIEGDLNPDNSD | |
| | VDKLFIQLVQTYNQLFEENPINASGVDAKAILSARLSKSRRLENLIAQLPGEKKNGLFGN | |
| | LIALSLGLTPNFKSNFDLAEDAKLQLSKDTYDDDLDNLLAQIGDQYADLFLAAKNLSDAI | |
| | LLSDILRVNTEITKAPLSASMIKRYDEHHQDLTLLKALVROOLPEKYKEIFFDQSKNGYA | |
| | GYIDGGASQEEFYKFIKPILEKMDGTEELLVKLNREDLLRKORTFDNGSIPHQIHLGELH | |
| | AILRRQEDFYPFLKDNREKIEKILTFRIPYYVGPLARGNSRFAWMTRKSEETITPWNFEE | |
| | VVDKGASAQSFIERMTNFDKNLPNEKVLPKHSLLYEYFTVYNELTKVKYVTEGMRKPAFL | |
| | SGEQKKAIVDLLFKTNRKVTVKQLKEDYFKKIECFDSVEISGVEDRFNASLGTYHDLLKI | |
| | IKDKDFLDNEENEDILEDIVLTLTLFEDREMIEERLKTYAHLFDDKVMKOLKRRRYTGWG | |
| | RLSRKLINGIRDKQSGKTILDFLKSDGFANRNFMQLIHDDSLTFKEDIQKAQVSGQGDSL | |
| | HEHIANLAGSPAIKKGILQTVKVVDELVKVMGRHKPENIVIEMARENQTTQKGQKNSRER | |
| | MKRIEEGIKELGSQILKEHPVENTQLQNEKLYLYYLONGRDMYVDQELDINRLSDYDVDH | |
| | IVPQSFLKDDSIDNKVLTRSDKNRGKSDNVPSEEVVKKMKNYWRQLLNAKLITORKEDNL | |
| | TKAERGGLSELDKAGFIKRQLVETROITKHVAQILDSRMNTKYDENDKLIREVKVITLKS | |
| | KLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDF | |
| | ATVRKVLSMPQVNIVKKTEVQTGGFSKESILPKRNSDKLIARKKDWDPKKYGGFDSPTVA | |
| | YSVLVVAKVEKGKSGSAGSAAGSGEFDRWGSQVQLVQSGGGLVQAGGSLRLSCAFSGRTF | |
| | SMYTMGWFRQAPGKEREFVAANRGRGLSPDIADSVNGRFTISRDNAKNTLYLOMDSLKPE | |
| | DTAVYYCAAASREDPPGYWGQGTTVTVSSKKLKSVKELLGITIMERSSFEKNPIDFLEAK | |
| | GYKEVKKDLIIKLPKYSLFELENGRKRMLASAGELQKGNELALPSKYVNFLYLASHYEKL | |
| | KGSPEDNEQKOLFVEQHKHYLDEIIEQISEFSKRVILADANLDKVLSAYNKHRDKPIREQ | |
| | AENIIHLFTLTNLGAPAAFKYFDTTIDRKRYTSTKEVLDATLIHQSITGLYETRIDLSQL | |
| | GGDGSPKKKRKVEDPKKKRKVDSGSETPGTSESATPESAEAGITGTWYNQSGSTFTVTAG | |
| | ADGNLTGQYENRAQGTGCONSPYTLTGRYNGTKLEWRVEWNNSTENCHSRTEWRGQYQGG | |
| | AEARINTOWNLTYEGGSGPATEQGODTFTKVKPSAAS | |
| L2 (DNA) | ATGGATAAGAAATACTCAATAGGCTTAGATATCGGCACAAATAGCGTCGGATGGGCGGTG | 70 |
| | ATCACTGATGAATATAAGGTTCCGTCTAAAAAGTTCAAGGTTCTGGGAAATACAGACCGC | |
| | CACAGTATCAAAAAAAATCTTATAGGGGCTCTTTTATTTGACAGTGGAGAGACAGCGGAA | |
| | GCGACTCGTCTCAAACGGACAGCTCGTAGAAGGTATACACGTCGGAAGAATCGTATTTGT | |
| | TATCTACAGGAGATTTTTTCAAATGAGATGGCGAAAGTAGATGATAGTTTCTTTCATCGA | |
| | CTTGAAGAGTCTTTTTTGGTGGAAGAAGACAAGAAGCATGAACGTCATCCTATTTTTGGA | |
| | AATATAGTAGATGAAGTTGCTTATCATGAGAAATATCCAACTATCTATCATCTGCGAAAA | |
| | AAATTGGTAGATTCTACTGATAAAGCGGATTTGCGCTTAATCTATTTGGCCTTAGCGCAT | |
| | ATGATTAAGTTTCGTGGTCATTTTTTGATTGAGGGAGATTTAAATCCTGATAATAGTGAT | |
| | GTGGACAAACTATTTATCCAGTTGGTACAAACCTACAATCAATTATTTGAAGAAAACCCT | |
| | ATTAACGCAAGTGGAGTAGATGCTAAAGCGATTCTTTCTGCACGATTGAGTAAATCAAGA | |
| | CGATTAGAAAATCTCATTGCTCAGCTCCCCGGTGAGAAGAAAAATGGCTTATTTGGGAAT | |
| | CTCATTGCTTTGTCATTGGGTTTGACCCCTAATTTTAAATCAAATTTTGATTTGGCAGAA | |
| | GATGCTAAATTACAGCTTTCAAAAGATACTTACGATGATGATTTAGATAATTTATTGGCG | |
| | CAAATTGGAGATCAATATGCTGATTTGTTTTTGGCAGCTAAGAATTTATCAGATGCTATT | |
| | TTACTTTCAGATATCCTAAGAGTAAATACTGAAATAACTAAGGCTCCCCTATCAGCTTCA | |
| | ATGATTAAACGCTACGATGAACATCATCAAGACTTGACTCTTTTAAAAGCTTTAGTTCGA | |
| | CAACAACTTCCAGAAAAGTATAAAGAAATCTTTTTTGATCAATCAAAAAACGGATATGCA | |
| | GGTTATATTGATGGGGGAGCTAGCCAAGAAGAATTTTATAAATTTATCAAACCAATTTTA | |
| | GAAAAAATGGATGGTACTGAGGAATTATTGGTGAAACTAAATCGTGAAGATTTGCTGCGC | |
| | AAGCAACGGACCTTTGACAACGGCTCTATTCCCCATCAAATTCACTTGGGTGAGCTGCAT | |
| | GCTATTTTGAGAAGACAAGAAGACTTTTATCCATTTTTAAAAGACAATCGTGAGAAGATT | |
| | GAAAAAATCTTGACTTTTCGAATTCCTTATTATGTTGGTCCATTGGCGCGTGGCAATAGT | |
| | CGTTTTGCATGGATGACTCGGAAGTCTGAAGAAACAATTACCCCATGGAATTTTGAAGAA | |
| | GTTGTCGATAAAGGTGCTTCAGCTCAATCATTTATTGAACGCATGACAAACTTTGATAAA | |
| | AATCTTCCAAATGAAAAAGTACTACCAAAACATAGTTTGCTTTATGAGTATTTTACGGTT | |
| | TATAACGAATTGACAAAGGTCAAATATGTTACTGAAGGAATGCGAAAACCAGCATTTCTT | |
| | TCAGGTGAACAGAAGAAAGCCATTGTTGATTTACTCTTCAAAACAAATCGAAAAGTAACC | |
| | GTTAAGCAATTAAAAGAAGATTATTTCAAAAAAATAGAATGTTTTGATAGTGTTGAAATT | |
| | TCAGGAGTTGAAGATAGATTTAATGCTTCATTAGGTACCTACCATGATTTGCTAAAAATT | |
| | ATTAAAGATAAAGATTTTTTGGATAATGAAGAAAATGAAGATATCTTAGAGGATATTGTT | |
| | TTAACATTGACCTTATTTGAAGATAGGGAGATGATTGAGGAAAGACTTAAAACATATGCT | |
| | CACCTCTTTGATGATAAGGTGATGAAACAGCTTAAACGTCGCCGTTATACTGGTTGGGGA | |
| | CGTTTGTCTCGAAAATTGATTAATGGTATTAGGGATAAGCAATCTGGCAAAACAATATTA | |
| | GATTTTTTGAAATCAGATGGTTTTGCCAATCGCAATTTTATGCAGCTGATCCATGATGAT | |
| | AGTTTGACATTTAAAGAAGACATTCAAAAAGCACAAGTGTCTGGACAAGGCGATAGTTTA | |
| | CATGAACATATTGCAAATTTAGCTGGTAGCCCTGCTATTAAAAAAGGTATTTTACAGACT | |
| | GTAAAAGTTGTTGATGAATTGGTCAAAGTAATGGGGCGGCATAAGCCAGAAAATATCGTT | |
| | ATTGAAATGGCACGTGAAAATCAGACAACTCAAAAGGGCCAGAAAAATTCGCGAGAGCGT | |
| | ATGAAACGAATCGAAGAAGGTATCAAAGAATTAGGAAGTCAGATTCTTAAAGAGCATCCT | |
| | GTTGAAAATACTCAATTGCAAAATGAAAAGCTCTATCTCTATTATCTCCAAAATGGAAGA | |
| | GACATGTATGTGGACCAAGAATTAGATATTAATCGTTTAAGTGATTATGATGTCGATCAC | |
| | ATTGTTCCACAAAGTTTCCTTAAAGACGATTCAATAGACAATAAGGTCTTAACGCGTTCT | |
| | GATAAAAATCGTGGTAAATCGGATAACGTTCCAAGTGAAGAAGTAGTCAAAAAGATGAAA | |
| | AACTATTGGAGACAACTTCTAAACGCCAAGTTAATCACTCAACGTAAGTTTGATAATTTA | |
| | ACGAAAGCTGAACGTGGAGGTTTGAGTGAACTTGATAAAGCTGGTTTTATCAAACGCCAA | |
| | TTGGTTGAAACTCGCCAAATCACTAAGCATGTGGCACAAATTTTGGATAGTCGCATGAAT | |
| | ACTAAATACGATGAAAATGATAAACTTATTCGAGAGGTTAAAGTGATTACCTTAAAATCT | |
| | AAATTAGTTTCTGACTTCCGAAAAGATTTCCAATTCTATAAAGTACGTGAGATTAACAAT | |
| | TACCATCATGCCCATGATGCGTATCTAAATGCCGTCGTTGGAACTGCTTTGATTAAGAAA | |
| | TATCCAAAACTTGAATCGGAGTTTGTCTATGGTGATTATAAAGTTTATGATGTTCGTAAA | |
| | ATGATTGCTAAGTCTGAGCAAGAAATAGGCAAAGCAACCGCAAAATATTTCTTTTACTCT | |
| | AATATCATGAACTTCTTCAAAACAGAAATTACACTTGCAAATGGAGAGATTCGCAAACGC | |
| | CCTCTAATCGAAACTAATGGGGAAACTGGAGAAATTGTCTGGGATAAAGGGCGAGATTTT | |
| | GCCACAGTGCGCAAAGTATTGTCCATGCCCCAAGTCAATATTGTCAAGAAAACAGAAGTA | |
| | CAGACAGGCGGATTCTCCAAGGAGTCAATTTTACCAAAAAGAAATTCGGACAAGCTTATT | |
| | GCTCGTAAAAAAGACTGGGATCCAAAAAAATATGGTGGTTTTGATAGTCCAACGGTAGCT | |
| | TATTCAGTCCTAGTGGTTGCTAAGGTGGAAAAAGGGAAATCGAAAGAGTCGGGAAGCGTC | |
| | TCTTCGGAACAGTTAGCGCAGTTCCGCTCACTGGATGATCGATGGGGATCCCAGGTTCAG | |
| | CTGGTTCAGAGCGGTGGTGGTCTGGTTCAGGCAGGCGGTAGCCTGCGTCTGAGCTGTGCA | |
| | TTTAGCGGTCGTACCTTTAGCATGTATACCATGGGTTGGTTTCGTCAGGCACCGGGTAAA | |
| | GAACGTGAATTTGTTGCAGCAAATCGTGGTCGTGGTCTGAGTCCGGATATTGCAGATAGC | |
| | GTTAATGGTCGTTTTACCATTAGCCGTGATAATGCCAAAAATACCCTGTACCTGCAGATG | |
| | GATAGCCTGAAACCGGAAGATACCGCAGTGTATTATTGTGCAGCAGCAAGCCGTGAAGAT | |
| | CCGCCTGGTTATTGGGGTCAGGGCACCACCGTTACCGTTAGCAGCAAGAAGTTAAAATCC | |
| | GTTAAAGAGTTACTAGGGATCACAATTATGGAAAGAAGTTCCTTTGAAAAAAATCCGATT | |
| | GACTTTTTAGAAGCTAAAGGATATAAGGAAGTTAAAAAAGACTTAATCATTAAACTACCT | |
| | AAATATAGTCTTTTTGAGTTAGAAAACGGTCGTAAACGGATGCTGGCTAGTGCCGGAGAA | |
| | TTACAAAAAGGAAATGAGCTGGCTCTGCCAAGCAAATATGTGAATTTTTTATATTTAGCT | |
| | AGTCATTATGAAAAGTTGAAGGGTAGTCCAGAAGATAACGAACAAAAACAATTGTTTGTG | |
| | GAGCAGCATAAGCATTATTTAGATGAGATTATTGAGCAAATCAGTGAATTTTCTAAGCGT | |
| | GTTATTTTAGCAGATGCCAATTTAGATAAAGTTCTTAGTGCATATAACAAACATAGAGAC | |
| | AAACCAATACGTGAACAAGCAGAAAATATTATTCATTTATTTACGTTGACGAATCTTGGA | |
| | GCTCCCGCTGCTTTTAAATATTTTGATACAACAATTGATCGTAAACGATATACGTCTACA | |
| | AAAGAAGTTTTAGATGCCACTCTTATCCATCAATCCATCACTGGTCTTTATGAAACACGC | |
| | ATTGATTTGAGTCAGCTAGGAGGTGACGGGTCACCTAAGAAAAAACGAAAAGTTGAGGAT | |
| | CCTAAAAAGAAACGAAAAGTTGATAGCGGTTCAGAGACCCCAGGAACTAGCGAGAGCGCT | |
| | ACACCGGAATCGGCGGAAGCGGGTATCACCGGCACGTGGTACAACCAGTCTGGTTCTACC | |
| | TTCACCGTTACCGCGGGTGCGGACGGTAACCTGACCGGTCAGTACGAAAACCGTGCGCAG | |
| | GGCACTGGTTGCCAGAACTCTCCGTACACCCTGACCGGTCGTTACAACGGTACCAAACTG | |
| | GAATGGCGTGTTGAATGGAACAACTCTACCGAAAACTGCCACTCTCGTACCGAATGGCGT | |
| | GGTCAGTACCAGGGTGGTGCGGAAGCGCGTATCAACACCCAGTGGAACCTGACCTACGAA | |
| | GGTGGTTCTGGTCCGGCGACCGAACAGGGTCAGGACACCTTCACCAAAGTTAAACCGTCT | |
| | GCGGCGTCTTAA | |
| L2 | Underlined: Nuclease | 71 |
| (annotated) | Bold: MBP + TEV site | |
| | Internal VHH: Italics | |
| | NLS: Bold and italic | |
| | His-tag: bold and underlined. | |
| | Linker: dot underlined | |
| | Monoavidin: dot underlined and bold | |
| | CACCATCACCATCACCATGGAAAAATCGAAGAAGGTAAACTGGTAATCTGGATTAACGGC | |
| | GATAAAGGCTATAACGGTCTCGCTGAAGTCGGTAAGAAATTCGAGAAAGATACCGGAATT | |
| | AAAGTCACCGTTGAGCATCCGGATAAACTGGAAGAGAAATTCCCACAGGTTGCGGCAACT | |
| | GGCGATGGCCCTGACATTATCTTCTGGGCACACGACCGCTTTGGTGGCTACGCTCAATCT | |
| | GGCCTGTTGGCTGAAATCACCCCGGACAAAGCGTTCCAGGACAAGCTGTATCCGTTTACC | |
| | TGGGATGCCGTACGTTACAACGGCAAGCTGATTGCTTACCCGATCGCTGTTGAAGCGTTA | |
| | TCGCTGATTTATAACAAAGATCTGCTGCCGAACCCGCCAAAAACCTGGGAAGAGATCCCG | |
| | GCGCTGGATAAAGAACTGAAAGCGAAAGGTAAGAGCGCGCTGATGTTCAACCTGCAAGAA | |
| | CCGTACTTCACCTGGCCGCTGATTGCTGCTGACGGGGGTTATGCGTTCAAGTATGAAAAC | |
| | GGCAAGTACGACATTAAAGACGTGGGCGTGGATAACGCTGGCGCGAAAGCGGGTCTGACC | |
| | TTCCTGGTTGACCTGATTAAAAACAAACACATGAATGCAGACACCGATTACTCCATCGCA | |
| | GAAGCTGCCTTTAATAAAGGCGAAACAGCGATGACCATCAACGGCCCGTGGGCATGGTCC | |
| | AACATCGACACCAGCAAAGTGAATTATGGTGTAACGGTACTGCCGACCTTCAAGGGTCAA | |
| | CCATCCAAACCGTTCGTTGGCGTGCTGAGCGCAGGTATTAACGCCGCCAGTCCGAACAAA | |
| | GAGCTGGCAAAAGAGTTCCTCGAAAACTATCTGCTGACTGATGAAGGTCTGGAAGCGGTT | |
| | AATAAAGACAAACCGCTGGGTGCCGTAGCGCTGAAGTCTTACGAGGAAGAGTTGGCGAAA | |
| | GATCCACGTATTGCCGCCACTATGGAAAACGCCCAGAAAGGTGAAATCATGCCGAACATC | |
| | CCGCAGATGTCCGCTTTCTGGTATGCCGTGCGTACTGCGGTGATCAACGCCGCCAGCGGT | |
| | CGTCAGACTGTCGATGAAGCCCTGAAAGACGCGCAGACTAATTCGAGCTCGAACAACAAC | |
| | AACACTAGTGAAAACCTGTATTTCCAGGGAGCAGCCTCGATGGATAAGAAATACTCAATA | |
| | GGCTTAGATATCGGCACAAATAGCGTCGGATGGGCGGTGATCACTGATGAATATAAGGTT | |
| | CCGTCTAAAAAGTTCAAGGTTCTGGGAAATACAGACCGCCACAGTATCAAAAAAAATCTT | |
| | ATAGGGGCTCTTTTATTTGACAGTGGAGAGACAGCGGAAGCGACTCGTCTCAAACGGACA | |
| | GCTCGTAGAAGGTATACACGTCGGAAGAATCGTATTTGTTATCTACAGGAGATTTTTTCA | |
| | AATGAGATGGCGAAAGTAGATGATAGTTTCTTTCATCGACTTGAAGAGTCTTTTTTGGTG | |
| | GAAGAAGACAAGAAGCATGAACGTCATCCTATTTTTGGAAATATAGTAGATGAAGTTGCT | |
| | TATCATGAGAAATATCCAACTATCTATCATCTGCGAAAAAAATTGGTAGATTCTACTGAT | |
| | AAAGCGGATTTGCGCTTAATCTATTTGGCCTTAGCGCATATGATTAAGTTTCGTGGTCAT | |
| | TTTTTGATTGAGGGAGATTTAAATCCTGATAATAGTGATGTGGACAAACTATTTATCCAG | |
| | TTGGTACAAACCTACAATCAATTATTTGAAGAAAACCCTATTAACGCAAGTGGAGTAGAT | |
| | GCTAAAGCGATTCTTTCTGCACGATTGAGTAAATCAAGACGATTAGAAAATCTCATTGCT | |
| | CAGCTCCCCGGTGAGAAGAAAAATGGCTTATTTGGGAATCTCATTGCTTTGTCATTGGGT | |
| | TTGACCCCTAATTTTAAATCAAATTTTGATTTGGCAGAAGATGCTAAATTACAGCTTTCA | |
| | AAAGATACTTACGATGATGATTTAGATAATTTATTGGCGCAAATTGGAGATCAATATGCT | |
| | GATTTGTTTTTGGCAGCTAAGAATTTATCAGATGCTATTTTACTTTCAGATATCCTAAGA | |
| | GTAAATACTGAAATAACTAAGGCTCCCCTATCAGCTTCAATGATTAAACGCTACGATGAA | |
| | CATCATCAAGACTTGACTCTTTTAAAAGCTTTAGTTCGACAACAACTTCCAGAAAAGTAT | |
| | AAAGAAATCTTTTTTGATCAATCAAAAAACGGATATGCAGGTTATATTGATGGGGGAGCT | |
| | AGCCAAGAAGAATTTTATAAATTTATCAAACCAATTTTAGAAAAAATGGATGGTACTGAG | |
| | GAATTATTGGTGAAACTAAATCGTGAAGATTTGCTGCGCAAGCAACGGACCTTTGACAAC | |
| | GGCTCTATTCCCCATCAAATTCACTTGGGTGAGCTGCATGCTATTTTGAGAAGACAAGAA | |
| | GACTTTTATCCATTTTTAAAAGACAATCGTGAGAAGATTGAAAAAATCTTGACTTTTCGA | |
| | ATTCCTTATTATGTTGGTCCATTGGCGCGTGGCAATAGTCGTTTTGCATGGATGACTCGG | |
| | AAGTCTGAAGAAACAATTACCCCATGGAATTTTGAAGAAGTTGTCGATAAAGGTGCTTCA | |
| | GCTCAATCATTTATTGAACGCATGACAAACTTTGATAAAAATCTTCCAAATGAAAAAGTA | |
| | CTACCAAAACATAGTTTGCTTTATGAGTATTTTACGGTTTATAACGAATTGACAAAGGTC | |
| | AAATATGTTACTGAAGGAATGCGAAAACCAGCATTTCTTTCAGGTGAACAGAAGAAAGCC | |
| | ATTGTTGATTTACTCTTCAAAACAAATCGAAAAGTAACCGTTAAGCAATTAAAAGAAGAT | |
| | TATTTCAAAAAAATAGAATGTTTTGATAGTGTTGAAATTTCAGGAGTTGAAGATAGATTT | |
| | AATGCTTCATTAGGTACCTACCATGATTTGCTAAAAATTATTAAAGATAAAGATTTTTTG | |
| | GATAATGAAGAAAATGAAGATATCTTAGAGGATATTGTTTTAACATTGACCTTATTTGAA | |
| | GATAGGGAGATGATTGAGGAAAGACTTAAAACATATGCTCACCTCTTTGATGATAAGGTG | |
| | ATGAAACAGCTTAAACGTCGCCGTTATACTGGTTGGGGACGTTTGTCTCGAAAATTGATT | |
| | AATGGTATTAGGGATAAGCAATCTGGCAAAACAATATTAGATTTTTTGAAATCAGATGGT | |
| | TTTGCCAATCGCAATTTTATGCAGCTGATCCATGATGATAGTTTGACATTTAAAGAAGAC | |
| | ATTCAAAAAGCACAAGTGTCTGGACAAGGCGATAGTTTACATGAACATATTGCAAATTTA | |
| | GCTGGTAGCCCTGCTATTAAAAAAGGTATTTTACAGACTGTAAAAGTTGTTGATGAATTG | |
| | GTCAAAGTAATGGGGCGGCATAAGCCAGAAAATATCGTTATTGAAATGGCACGTGAAAAT | |
| | CAGACAACTCAAAAGGGCCAGAAAAATTCGCGAGAGCGTATGAAACGAATCGAAGAAGGT | |
| | ATCAAAGAATTAGGAAGTCAGATTCTTAAAGAGCATCCTGTTGAAAATACTCAATTGCAA | |
| | AATGAAAAGCTCTATCTCTATTATCTCCAAAATGGAAGAGACATGTATGTGGACCAAGAA | |
| | TTAGATATTAATCGTTTAAGTGATTATGATGTCGATCACATTGTTCCACAAAGTTTCCTT | |
| | AAAGACGATTCAATAGACAATAAGGTCTTAACGCGTTCTGATAAAAATCGTGGTAAATCG | |
| | GATAACGTTCCAAGTGAAGAAGTAGTCAAAAAGATGAAAAACTATTGGAGACAACTTCTA | |
| | AACGCCAAGTTAATCACTCAACGTAAGTTTGATAATTTAACGAAAGCTGAACGTGGAGGT | |
| | TTGAGTGAACTTGATAAAGCTGGTTTTATCAAACGCCAATTGGTTGAAACTCGCCAAATC | |
| | ACTAAGCATGTGGCACAAATTTTGGATAGTCGCATGAATACTAAATACGATGAAAATGAT | |
| | AAACTTATTCGAGAGGTTAAAGTGATTACCTTAAAATCTAAATTAGTTTCTGACTTCCGA | |
| | AAAGATTTCCAATTCTATAAAGTACGTGAGATTAACAATTACCATCATGCCCATGATGCG | |
| | TATCTAAATGCCGTCGTTGGAACTGCTTTGATTAAGAAATATCCAAAACTTGAATCGGAG | |
| | TTTGTCTATGGTGATTATAAAGTTTATGATGTTCGTAAAATGATTGCTAAGTCTGAGCAA | |
| | GAAATAGGCAAAGCAACCGCAAAATATTTCTTTTACTCTAATATCATGAACTTCTTCAAA | |
| | ACAGAAATTACACTTGCAAATGGAGAGATTCGCAAACGCCCTCTAATCGAAACTAATGGG | |
| | GAAACTGGAGAAATTGTCTGGGATAAAGGGCGAGATTTTGCCACAGTGCGCAAAGTATTG | |
| | TCCATGCCCCAAGTCAATATTGTCAAGAAAACAGAAGTACAGACAGGCGGATTCTCCAAG | |
| | GAGTCAATTTTACCAAAAAGAAATTCGGACAAGCTTATTGCTCGTAAAAAAGACTGGGAT | |
| | CCAAAAAAATATGGTGGTTTTGATAGTCCAACGGTAGCTTATTCAGTCCTAGTGGTTGCT | |
| | 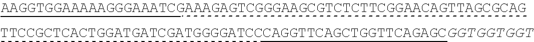 | |
| | CTGGTTCAGGCAGGCGGTAGCCTGCGTCTGAGCTGTGCATTTAGCGGTCGTACCTTTAGC | |
| | ATGTATACCATGGGTTGGTTTCGTCAGGCACCGGGTAAAGAACGTGAATTTGTTGCAGCA | |
| | AATCGTGGTCGTGGTCTGAGTCCGGATATTGCAGATAGCGTTAATGGTCGTTTTACCATT | |
| | AGCCGTGATAATGCCAAAAATACCCTGTACCTGCAGATGGATAGCCTGAAACCGGAAGAT | |
| | ACCGCAGTGTATTATTGTGCAGCAGCAAGCCGTGAAGATCCGCCTGGTTATTGGGGTCAG | |
| | GGCACCACCGTTACCGTTAGCAGCAAGAAGTTAAAATCCGTTAAAGAGTTACTAGGGATC | |
| | ACAATTATGGAAAGAAGTTCCTTTGAAAAAAATCCGATTGACTTTTTAGAAGCTAAAGGA | |
| | TATAAGGAAGTTAAAAAAGACTTAATCATTAAACTACCTAAATATAGTCTTTTTGAGTTA | |
| | GAAAACGGTCGTAAACGGATGCTGGCTAGTGCCGGAGAATTACAAAAAGGAAATGAGCTG | |
| | GCTCTGCCAAGCAAATATGTGAATTTTTTATATTTAGCTAGTCATTATGAAAAGTTGAAG | |
| | GGTAGTCCAGAAGATAACGAACAAAAACAATTGTTTGTGGAGCAGCATAAGCATTATTTA | |
| | GATGAGATTATTGAGCAAATCAGTGAATTTTCTAAGCGTGTTATTTTAGCAGATGCCAAT | |
| | TTAGATAAAGTTCTTAGTGCATATAACAAACATAGAGACAAACCAATACGTGAACAAGCA | |
| | GAAAATATTATTCATTTATTTACGTTGACGAATCTTGGAGCTCCCGCTGCTTTTAAATAT | |
| | TTTGATACAACAATTGATCGTAAACGATATACGTCTACAAAAGAAGTTTTAGATGCCACT | |
| | CTTATCCATCAATCCATCACTGGTCTTTATGAAACACGCATTGATTTGAGTCAGCTAGGA | |
| | GGTGACGGGTCACCTAAGAAAAAACGAAAAGTTGAGGATCCTAAAAAGAAACGAAAAGTT | |
| | 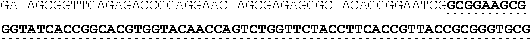 | |
| | 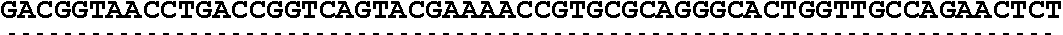 | |
| | 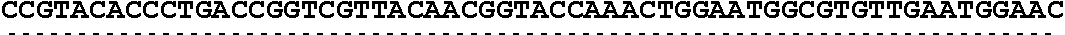 | |
| | 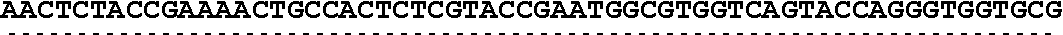 | |
| | 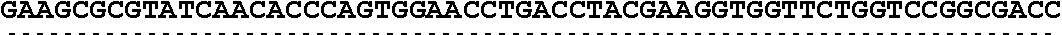 | |
| | 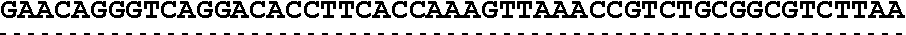 | |
| L2 (a.a.) | MDKKYSIGLDIGTNSVGWAVITDEYKVPSKKFKVLGNTDRHSIKKNLIGALLFDSGETAE | 72 |
| | ATRLKRTARRRYTRRKNRICYLQEIFSNEMAKVDDSFFHRLEESFLVEEDKKHERHPIFG | |
| | NIVDEVAYHEKYPTIYHLRKKLVDSTDKADLRLIYLALAHMIKERGHFLIEGDLNPDNSD | |
| | VDKLFIQLVQTYNQLFEENPINASGVDAKAILSARLSKSRRLENLIAQLPGEKKNGLFGN | |
| | LIALSLGLTPNFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEIFFDQSKNGYA | |
| | GYIDGGASQEEFYKFIKPILEKMDGTEELLVKLNREDLLRKORTFDNGSIPHQIHLGELH | |
| | AILRRQEDFYPFLKDNREKIEKILTFRIPYYVGPLARGNSRFAWMTRKSEETITPWNFEE | |
| | VVDKGASAQSFIERMTNFDKNLPNEKVLPKHSLLYEYFTVYNELTKVKYVTEGMRKPAFL | |
| | SGEQKKAIVDLLFKTNRKVTVKQLKEDYFKKIECFDSVEISGVEDRFNASLGTYHDLLKI | |
| | IKDKDFLDNEENEDILEDIVLTLTLFEDREMIEERLKTYAHLFDDKVMKOLKRRRYTGWG | |
| | RLSRKLINGIRDKQSGKTILDFLKSDGFANRNFMQLIHDDSLTFKEDIQKAQVSGQGDSL | |
| | HEHIANLAGSPAIKKGILQTVKVVDELVKVMGRHKPENIVIEMARENOTTQKGQKNSRER | |
| | MKRIEEGIKELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWROLLNAKLITORKFDNL | |
| | TKAERGGLSELDKAGFIKRQLVETROITKHVAQILDSRMNTKYDENDKLIREVKVITLKS | |
| | KLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDF | |
| | ATVRKVLSMPQVNIVKKTEVQTGGFSKESILPKRNSDKLIARKKDWDPKKYGGFDSPTVA | |
| | YSVLVVAKVEKGKSKESGSVSSEQLAQFRSLDDRWGSQVOLVOSGGGLVQAGGSLRLSCA | |
| | FSGRTFSMYTMGWFRQAPGKEREFVAANRGRGLSPDIADSVNGRFTISRDNAKNTLYLQM | |
| | DSLKPEDTAVYYCAAASREDPPGYWGQGTTVTVSSKKLKSVKELLGITIMERSSFEKNPI | |
| | DFLEAKGYKEVKKDLIIKLPKYSLFELENGRKRMLASAGELQKGNELALPSKYVNFLYLA | |
| | SHYEKLKGSPEDNEQKOLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGLYETR | |
| | IDLSQLGGDGSPKKKRKVEDPKKKRKVDSGSETPGTSESATPESAEAGITGTWYNQSGST | |
| | FTVTAGADGNLTGQYENRAQGTGCONSPYTLTGRYNGTKLEWRVEWNNSTENCHSRTEWR | |
| | GQYQGGAEARINTOWNLTYEGGSGPATEQGODTFTKVKPSAAS | |
| L3 (DNA) | AGCGCCTGATGCGGTATTTTCTCCTTACGCATCTGTGCGGTATTTCACACCGCAATGGTG | 73 |
| | CACTCTCAGTACAATCTGCTCTGATGCCGCATAGTTAAGCCAGTATACACTCCGCTATCG | |
| | CTACGTGACTGGGTCATGGCTGCGCCCCGACACCCGCCAACACCCGCTGACGCGCCCTGA | |
| | CGGGCTTGTCTGCTCCCGGCATCCGCTTACAGACAAGCTGTGACCGTCTCCGGGAGCTGC | |
| | ATGTGTCAGAGGTTTTCACCGTCATCACCGAAACGCGCGAGGCAGCTGCGGTAAAGCTCA | |
| | TCAGCGTGGTCGTGAAGCGATTCACAGATGTCTGCCTGTTCATCCGCGTCCAGCTCGTTG | |
| | AGTTTCTCCAGAAGCGTTAATGTCTGGCTTCTGATAAAGCGGGCCATGTTAAGGGCGGTT | |
| | TTTTCCTGTTTGGTCACTGATGCCTCCGTGTAAGGGGGATTTCTGTTCATGGGGGTAATG | |
| | ATACCGATGAAACGAGAGAGGATGCTCACGATACGGGTTACTGATGATGAACATGCCCGG | |
| | TTACTGGAACGTTGTGAGGGTAAACAACTGGCGGTATGGATGCGGCGGGACCAGAGAAAA | |
| | ATCACTCAGGGTCAATGCCAGCGCTTCGTTAATACAGATGTAGGTGTTCCACAGGGTAGC | |
| | CAGCAGCATCCTGCGATGCAGATCCGGAACATAATGGTGCAGGGCGCTGACTTCCGCGTT | |
| | TCCAGACTTTACGAAACACGGAAACCGAAGACCATTCATGTTGTTGCTCAGGTCGCAGAC | |
| | GTTTTGCAGCAGCAGTCGCTTCACGTTCGCTCGCGTATCGGTGATTCATTCTGCTAACCA | |
| | GTAAGGCAACCCCGCCAGCCTAGCCGGGTCCTCAACGACAGGAGCACGATCATGCGCACC | |
| | CGTGGGGCCGCCATGCCGGCGATAATGGCCTGCTTCTCGCCGAAACGTTTGGTGGCGGGA | |
| | CCAGTGACGAAGGCTTGAGCGAGGGCGTGCAAGATTCCGAATACCGCAAGCGACAGGCCG | |
| | ATCATCGTCGCGCTCCAGCGAAAGCGGTCCTCGCCGAAAATGACCCAGAGCGCTGCCGGC | |
| | ACCTGTCCTACGAGTTGCATGATAAAGAAGACAGTCATAAGTGCGGCGACGATAGTCATG | |
| | CCCCGCGCCCACCGGAAGGAGCTGACTGGGTTGAAGGCTCTCAAGGGCATCGGTCGAGAT | |
| | CCCGGTGCCTAATGAGTGAGCTAACTTACATTAATTGCGTTGCGCTCACTGCCCGCTTTC | |
| | CAGTCGGGAAACCTGTCGTGCCAGCTGCATTAATGAATCGGCCAACGCGCGGGGAGAGGC | |
| | GGTTTGCGTATTGGGCGCCAGGGTGGTTTTTCTTTTCACCAGTGAGACGGGCAACAGCTG | |
| | ATTGCCCTTCACCGCCTGGCCCTGAGAGAGTTGCAGCAAGCGGTCCACGCTGGTTTGCCC | |
| | CAGCAGGCGAAAATCCTGTTTGATGGTGGTTAACGGCGGGATATAACATGAGCTGTCTTC | |
| | GGTATCGTCGTATCCCACTACCGAGATATCCGCACCAACGCGCAGCCCGGACTCGGTAAT | |
| | GGCGCGCATTGCGCCCAGCGCCATCTGATCGTTGGCAACCAGCATCGCAGTGGGAACGAT | |
| | GCCCTCATTCAGCATTTGCATGGTTTGTTGAAAACCGGACATGGCACTCCAGTCGCCTTC | |
| | CCGTTCCGCTATCGGCTGAATTTGATTGCGAGTGAGATATTTATGCCAGCCAGCCAGACG | |
| | CAGACGCGCCGAGACAGAACTTAATGGGCCCGCTAACAGCGCGATTTGCTGGTGACCCAA | |
| | TGCGACCAGATGCTCCACGCCCAGTCGCGTACCGTCTTCATGGGAGAAAATAATACTGTT | |
| | GATGGGTGTCTGGTCAGAGACATCAAGAAATAACGCCGGAACATTAGTGCAGGCAGCTTC | |
| | CACAGCAATGGCATCCTGGTCATCCAGCGGATAGTTAATGATCAGCCCACTGACGCGTTG | |
| | CGCGAGAAGATTGTGCACCGCCGCTTTACAGGCTTCGACGCCGCTTCGTTCTACCATCGA | |
| | CACCACCACGCTGGCACCCAGTTGATCGGCGCGAGATTTAATCGCCGCGACAATTTGCGA | |
| | CGGCGCGTGCAGGGCCAGACTGGAGGTGGCAACGCCAATCAGCAACGACTGTTTGCCCGC | |
| | CAGTTGTTGTGCCACGCGGTTGGGAATGTAATTCAGCTCCGCCATCGCCGCTTCCACTTT | |
| | TTCCCGCGTTTTCGCAGAAACGTGGCTGGCCTGGTTCACCACGCGGGAAACGGTCTGATA | |
| | AGAGACACCGGCATACTCTGCGACATCGTATAACGTTACTGGTTTCACATTCACCACCCT | |
| | GAATTGACTCTCTTCCGGGCGCTATCATGCCATACCGCGAAAGGTTTTGCGCCATTCGAT | |
| | GGTGTCCGGGATCTCGACGCTCTCCCTTATGCGACTCCTGCATTAGGAAGCAGCCCAGTA | |
| | GTAGGTTGAGGCCGTTGAGCACCGCCGCCGCAAGGAATGGTGCATGCAAGGAGATGGCGC | |
| | CCAACAGTCCCCCGGCCACGGGGCCTGCCACCATACCCACGCCGAAACAAGCGCTCATGA | |
| | GCCCGAAGTGGCGAGCCCGATCTTCCCCATCGGTGATGTCGGCGATATAGGCGCCAGCAA | |
| | CCGCACCTGTGGCGCCGGTGATGCCGGCCACGATGCGTCCGGCGTAGAGGATCGAGATCT | |
| | CGATCCCGCGAAATTAATACGACTCACTATAGGGGAATTGTGAGCGGATAACAATTCCCC | |
| | TCTAGAAATAATTTTGTTTAACTTTAAGAAGGAGATATACATATGCACCATCACCATCAC | |
| | CATGGAAAAATCGAAGAAGGTAAACTGGTAATCTGGATTAACGGCGATAAAGGCTATAAC | |
| | GGTCTCGCTGAAGTCGGTAAGAAATTCGAGAAAGATACCGGAATTAAAGTCACCGTTGAG | |
| | CATCCGGATAAACTGGAAGAGAAATTCCCACAGGTTGCGGCAACTGGCGATGGCCCTGAC | |
| | ATTATCTTCTGGGCACACGACCGCTTTGGTGGCTACGCTCAATCTGGCCTGTTGGCTGAA | |
| | ATCACCCCGGACAAAGCGTTCCAGGACAAGCTGTATCCGTTTACCTGGGATGCCGTACGT | |
| | TACAACGGCAAGCTGATTGCTTACCCGATCGCTGTTGAAGCGTTATCGCTGATTTATAAC | |
| | AAAGATCTGCTGCCGAACCCGCCAAAAACCTGGGAAGAGATCCCGGCGCTGGATAAAGAA | |
| | CTGAAAGCGAAAGGTAAGAGCGCGCTGATGTTCAACCTGCAAGAACCGTACTTCACCTGG | |
| | CCGCTGATTGCTGCTGACGGGGGTTATGCGTTCAAGTATGAAAACGGCAAGTACGACATT | |
| | AAAGACGTGGGCGTGGATAACGCTGGCGCGAAAGCGGGTCTGACCTTCCTGGTTGACCTG | |
| | ATTAAAAACAAACACATGAATGCAGACACCGATTACTCCATCGCAGAAGCTGCCTTTAAT | |
| | AAAGGCGAAACAGCGATGACCATCAACGGCCCGTGGGCATGGTCCAACATCGACACCAGC | |
| | AAAGTGAATTATGGTGTAACGGTACTGCCGACCTTCAAGGGTCAACCATCCAAACCGTTC | |
| | GTTGGCGTGCTGAGCGCAGGTATTAACGCCGCCAGTCCGAACAAAGAGCTGGCAAAAGAG | |
| | TTCCTCGAAAACTATCTGCTGACTGATGAAGGTCTGGAAGCGGTTAATAAAGACAAACCG | |
| | CTGGGTGCCGTAGCGCTGAAGTCTTACGAGGAAGAGTTGGCGAAAGATCCACGTATTGCC | |
| | GCCACTATGGAAAACGCCCAGAAAGGTGAAATCATGCCGAACATCCCGCAGATGTCCGCT | |
| | TTCTGGTATGCCGTGCGTACTGCGGTGATCAACGCCGCCAGCGGTCGTCAGACTGTCGAT | |
| | GAAGCCCTGAAAGACGCGCAGACTAATTCGAGCTCGAACAACAACAACACTAGTGAAAAC | |
| | CTGTATTTCCAGGGAGCAGCCTCGATGGATAAGAAATACTCAATAGGCTTAGATATCGGC | |
| | ACAAATAGCGTCGGATGGGCGGTGATCACTGATGAATATAAGGTTCCGTCTAAAAAGTTC | |
| | AAGGTTCTGGGAAATACAGACCGCCACAGTATCAAAAAAAATCTTATAGGGGCTCTTTTA | |
| | TTTGACAGTGGAGAGACAGCGGAAGCGACTCGTCTCAAACGGACAGCTCGTAGAAGGTAT | |
| | ACACGTCGGAAGAATCGTATTTGTTATCTACAGGAGATTTTTTCAAATGAGATGGCGAAA | |
| | GTAGATGATAGTTTCTTTCATCGACTTGAAGAGTCTTTTTTGGTGGAAGAAGACAAGAAG | |
| | CATGAACGTCATCCTATTTTTGGAAATATAGTAGATGAAGTTGCTTATCATGAGAAATAT | |
| | CCAACTATCTATCATCTGCGAAAAAAATTGGTAGATTCTACTGATAAAGCGGATTTGCGC | |
| | TTAATCTATTTGGCCTTAGCGCATATGATTAAGTTTCGTGGTCATTTTTTGATTGAGGGA | |
| | GATTTAAATCCTGATAATAGTGATGTGGACAAACTATTTATCCAGTTGGTACAAACCTAC | |
| | AATCAATTATTTGAAGAAAACCCTATTAACGCAAGTGGAGTAGATGCTAAAGCGATTCTT | |
| | TCTGCACGATTGAGTAAATCAAGACGATTAGAAAATCTCATTGCTCAGCTCCCCGGTGAG | |
| | AAGAAAAATGGCTTATTTGGGAATCTCATTGCTTTGTCATTGGGTTTGACCCCTAATTTT | |
| | AAATCAAATTTTGATTTGGCAGAAGATGCTAAATTACAGCTTTCAAAAGATACTTACGAT | |
| | GATGATTTAGATAATTTATTGGCGCAAATTGGAGATCAATATGCTGATTTGTTTTTGGCA | |
| | GCTAAGAATTTATCAGATGCTATTTTACTTTCAGATATCCTAAGAGTAAATACTGAAATA | |
| | ACTAAGGCTCCCCTATCAGCTTCAATGATTAAACGCTACGATGAACATCATCAAGACTTG | |
| | ACTCTTTTAAAAGCTTTAGTTCGACAACAACTTCCAGAAAAGTATAAAGAAATCTTTTTT | |
| | GATCAATCAAAAAACGGATATGCAGGTTATATTGATGGGGGAGCTAGCCAAGAAGAATTT | |
| | TATAAATTTATCAAACCAATTTTAGAAAAAATGGATGGTACTGAGGAATTATTGGTGAAA | |
| | CTAAATCGTGAAGATTTGCTGCGCAAGCAACGGACCTTTGACAACGGCTCTATTCCCCAT | |
| | CAAATTCACTTGGGTGAGCTGCATGCTATTTTGAGAAGACAAGAAGACTTTTATCCATTT | |
| | TTAAAAGACAATCGTGAGAAGATTGAAAAAATCTTGACTTTTCGAATTCCTTATTATGTT | |
| | GGTCCATTGGCGCGTGGCAATAGTCGTTTTGCATGGATGACTCGGAAGTCTGAAGAAACA | |
| | ATTACCCCATGGAATTTTGAAGAAGTTGTCGATAAAGGTGCTTCAGCTCAATCATTTATT | |
| | GAACGCATGACAAACTTTGATAAAAATCTTCCAAATGAAAAAGTACTACCAAAACATAGT | |
| | TTGCTTTATGAGTATTTTACGGTTTATAACGAATTGACAAAGGTCAAATATGTTACTGAA | |
| | GGAATGCGAAAACCAGCATTTCTTTCAGGTGAACAGAAGAAAGCCATTGTTGATTTACTC | |
| | TTCAAAACAAATCGAAAAGTAACCGTTAAGCAATTAAAAGAAGATTATTTCAAAAAAATA | |
| | GAATGTTTTGATAGTGTTGAAATTTCAGGAGTTGAAGATAGATTTAATGCTTCATTAGGT | |
| | ACCTACCATGATTTGCTAAAAATTATTAAAGATAAAGATTTTTTGGATAATGAAGAAAAT | |
| | GAAGATATCTTAGAGGATATTGTTTTAACATTGACCTTATTTGAAGATAGGGAGATGATT | |
| | GAGGAAAGACTTAAAACATATGCTCACCTCTTTGATGATAAGGTGATGAAACAGCTTAAA | |
| | CGTCGCCGTTATACTGGTTGGGGACGTTTGTCTCGAAAATTGATTAATGGTATTAGGGAT | |
| | AAGCAATCTGGCAAAACAATATTAGATTTTTTGAAATCAGATGGTTTTGCCAATCGCAAT | |
| | TTTATGCAGCTGATCCATGATGATAGTTTGACATTTAAAGAAGACATTCAAAAAGCACAA | |
| | GTGTCTGGACAAGGCGATAGTTTACATGAACATATTGCAAATTTAGCTGGTAGCCCTGCT | |
| | ATTAAAAAAGGTATTTTACAGACTGTAAAAGTTGTTGATGAATTGGTCAAAGTAATGGGG | |
| | CGGCATAAGCCAGAAAATATCGTTATTGAAATGGCACGTGAAAATCAGACAACTCAAAAG | |
| | GGCCAGAAAAATTCGCGAGAGCGTATGAAACGAATCGAAGAAGGTATCAAAGAATTAGGA | |
| | AGTCAGATTCTTAAAGAGCATCCTGTTGAAAATACTCAATTGCAAAATGAAAAGCTCTAT | |
| | CTCTATTATCTCCAAAATGGAAGAGACATGTATGTGGACCAAGAATTAGATATTAATCGT | |
| | TTAAGTGATTATGATGTCGATCACATTGTTCCACAAAGTTTCCTTAAAGACGATTCAATA | |
| | GACAATAAGGTCTTAACGCGTTCTGATAAAAATCGTGGTAAATCGGATAACGTTCCAAGT | |
| | GAAGAAGTAGTCAAAAAGATGAAAAACTATTGGAGACAACTTCTAAACGCCAAGTTAATC | |
| | ACTCAACGTAAGTTTGATAATTTAACGAAAGCTGAACGTGGAGGTTTGAGTGAACTTGAT | |
| | AAAGCTGGTTTTATCAAACGCCAATTGGTTGAAACTCGCCAAATCACTAAGCATGTGGCA | |
| | CAAATTTTGGATAGTCGCATGAATACTAAATACGATGAAAATGATAAACTTATTCGAGAG | |
| | GTTAAAGTGATTACCTTAAAATCTAAATTAGTTTCTGACTTCCGAAAAGATTTCCAATTC | |
| | TATAAAGTACGTGAGATTAACAATTACCATCATGCCCATGATGCGTATCTAAATGCCGTC | |
| | GTTGGAACTGCTTTGATTAAGAAATATCCAAAACTTGAATCGGAGTTTGTCTATGGTGAT | |
| | TATAAAGTTTATGATGTTCGTAAAATGATTGCTAAGTCTGAGCAAGAAATAGGCAAAGCA | |
| | ACCGCAAAATATTTCTTTTACTCTAATATCATGAACTTCTTCAAAACAGAAATTACACTT | |
| | GCAAATGGAGAGATTCGCAAACGCCCTCTAATCGAAACTAATGGGGAAACTGGAGAAATT | |
| | GTCTGGGATAAAGGGCGAGATTTTGCCACAGTGCGCAAAGTATTGTCCATGCCCCAAGTC | |
| | AATATTGTCAAGAAAACAGAAGTACAGACAGGCGGATTCTCCAAGGAGTCAATTTTACCA | |
| | AAAAGAAATTCGGACAAGCTTATTGCTCGTAAAAAAGACTGGGATCCAAAAAAATATGGT | |
| | GGTTTTGATAGTCCAACGGTAGCTTATTCAGTCCTAGTGGTTGCTAAGGTGGAAAAAGGG | |
| | AAATCGCAGCAGTATTATAGCTATCGCACCAAGAAGTTAAAATCCGTTAAAGAGTTACTA | |
| | GGGATCACAATTATGGAAAGAAGTTCCTTTGAAAAAAATCCGATTGACTTTTTAGAAGCT | |
| | AAAGGATATAAGGAAGTTAAAAAAGACTTAATCATTAAACTACCTAAATATAGTCTTTTT | |
| | GAGTTAGAAAACGGTCGTAAACGGATGCTGGCTAGTGCCGGAGAATTACAAAAAGGAAAT | |
| | GAGCTGGCTCTGCCAAGCAAATATGTGAATTTTTTATATTTAGCTAGTCATTATGAAAAG | |
| | TTGAAGGGTAGTCCAGAAGATAACGAACAAAAACAATTGTTTGTGGAGCAGCATAAGCAT | |
| | TATTTAGATGAGATTATTGAGCAAATCAGTGAATTTTCTAAGCGTGTTATTTTAGCAGAT | |
| | GCCAATTTAGATAAAGTTCTTAGTGCATATAACAAACATAGAGACAAACCAATACGTGAA | |
| | CAAGCAGAAAATATTATTCATTTATTTACGTTGACGAATCTTGGAGCTCCCGCTGCTTTT | |
| | AAATATTTTGATACAACAATTGATCGTAAACGATATACGTCTACAAAAGAAGTTTTAGAT | |
| | GCCACTCTTATCCATCAATCCATCACTGGTCTTTATGAAACACGCATTGATTTGAGTCAG | |
| | CTAGGAGGTGACGGGTCACCTAAGAAAAAACGAAAAGTTGAGGATCCTAAAAAGAAACGA | |
| | AAAGTTGATAGCGGTTCAGAGACCCCAGGAACTAGCGAGAGCGCTACACCGGAATCGGCG | |
| | GAAGCGGGTATCACCGGCACGTGGTACAACCAGTCTGGTTCTACCTTCACCGTTACCGCG | |
| | GGTGCGGACGGTAACCTGACCGGTCAGTACGAAAACCGTGCGCAGGGCACTGGTTGCCAG | |
| | AACTCTCCGTACACCCTGACCGGTCGTTACAACGGTACCAAACTGGAATGGCGTGTTGAA | |
| | TGGAACAACTCTACCGAAAACTGCCACTCTCGTACCGAATGGCGTGGTCAGTACCAGGGT | |
| | GGTGCGGAAGCGCGTATCAACACCCAGTGGAACCTGACCTACGAAGGTGGTTCTGGTCCG | |
| | GCGACCGAACAGGGTCAGGACACCTTCACCAAAGTTAAACCGTCTGCGGCGTCTTAAGCG | |
| | GCCGCACTCGAGCACCACCACCACCACCACTGAGATCCGGCTGCTAACAAAGCCCGAAAG | |
| | GAAGCTGAGTTGGCTGCTGCCACCGCTGAGCAATAACTAGCATAACCCCTTGGGGCCTCT | |
| | AAACGGGTCTTGAGGGGTTTTTTGCTGAAAGGAGGAACTATATCCGGATTGGCGAATGGG | |
| | ACGCGCCCTGTAGCGGCGCATTAAGCGCGGCGGGTGTGGTGGTTACGCGCAGCGTGACCG | |
| | CTACACTTGCCAGCGCCCTAGCGCCCGCTCCTTTCGCTTTCTTCCCTTCCTTTCTCGCCA | |
| | CGTTCGCCGGCTTTCCCCGTCAAGCTCTAAATCGGGGGCTCCCTTTAGGGTTCCGATTTA | |
| | GTGCTTTACGGCACCTCGACCCCAAAAAACTTGATTAGGGTGATGGTTCACGTAGTGGGC | |
| | CATCGCCCTGATAGACGGTTTTTCGCCCTTTGACGTTGGAGTCCACGTTCTTTAATAGTG | |
| | GACTCTTGTTCCAAACTGGAACAACACTCAACCCTATCTCGGTCTATTCTTTTGATTTAT | |
| | AAGGGATTTTGCCGATTTCGGCCTATTGGTTAAAAAATGAGCTGATTTAACAAAAATTTA | |
| | ACGCGAATTTTAACAAAATATTAACGCTTACAATTTAGGTGGCACTTTTCGGGGAAATGT | |
| | GCGCGGAACCCCTATTTGTTTATTTTTCTAAATACATTCAAATATGTATCCGCTCATGAA | |
| | TTAATTCTTAGAAAAACTCATCGAGCATCAAATGAAACTGCAATTTATTCATATCAGGAT | |
| | TATCAATACCATATTTTTGAAAAAGCCGTTTCTGTAATGAAGGAGAAAACTCACCGAGGC | |
| | AGTTCCATAGGATGGCAAGATCCTGGTATCGGTCTGCGATTCCGACTCGTCCAACATCAA | |
| | TACAACCTATTAATTTCCCCTCGTCAAAAATAAGGTTATCAAGTGAGAAATCACCATGAG | |
| | TGACGACTGAATCCGGTGAGAATGGCAAAAGTTTATGCATTTCTTTCCAGACTTGTTCAA | |
| | CAGGCCAGCCATTACGCTCGTCATCAAAATCACTCGCATCAACCAAACCGTTATTCATTC | |
| | GTGATTGCGCCTGAGCGAGACGAAATACGCGATCGCTGTTAAAAGGACAATTACAAACAG | |
| | GAATCGAATGCAACCGGCGCAGGAACACTGCCAGCGCATCAACAATATTTTCACCTGAAT | |
| | CAGGATATTCTTCTAATACCTGGAATGCTGTTTTCCCGGGGATCGCAGTGGTGAGTAACC | |
| | ATGCATCATCAGGAGTACGGATAAAATGCTTGATGGTCGGAAGAGGCATAAATTCCGTCA | |
| | GCCAGTTTAGTCTGACCATCTCATCTGTAACATCATTGGCAACGCTACCTTTGCCATGTT | |
| | TCAGAAACAACTCTGGCGCATCGGGCTTCCCATACAATCGATAGATTGTCGCACCTGATT | |
| | GCCCGACATTATCGCGAGCCCATTTATACCCATATAAATCAGCATCCATGTTGGAATTTA | |
| | ATCGCGGCCTAGAGCAAGACGTTTCCCGTTGAATATGGCTCATAACACCCCTTGTATTAC | |
| | TGTTTATGTAAGCAGACAGTTTTATTGTTCATGACCAAAATCCCTTAACGTGAGTTTTCG | |
| | TTCCACTGAGCGTCAGACCCCGTAGAAAAGATCAAAGGATCTTCTTGAGATCCTTTTTTT | |
| | CTGCGCGTAATCTGCTGCTTGCAAACAAAAAAACCACCGCTACCAGCGGTGGTTTGTTTG | |
| | CCGGATCAAGAGCTACCAACTCTTTTTCCGAAGGTAACTGGCTTCAGCAGAGCGCAGATA | |
| | CCAAATACTGTCCTTCTAGTGTAGCCGTAGTTAGGCCACCACTTCAAGAACTCTGTAGCA | |
| | CCGCCTACATACCTCGCTCTGCTAATCCTGTTACCAGTGGCTGCTGCCAGTGGCGATAAG | |
| | TCGTGTCTTACCGGGTTGGACTCAAGACGATAGTTACCGGATAAGGCGCAGCGGTCGGGC | |
| | TGAACGGGGGGTTCGTGCACACAGCCCAGCTTGGAGCGAACGACCTACACCGAACTGAGA | |
| | TACCTACAGCGTGAGCTATGAGAAAGCGCCACGCTTCCCGAAGGGAGAAAGGCGGACAGG | |
| | TATCCGGTAAGCGGCAGGGTCGGAACAGGAGAGCGCACGAGGGAGCTTCCAGGGGGAAAC | |
| | GCCTGGTATCTTTATAGTCCTGTCGGGTTTCGCCACCTCTGACTTGAGCGTCGATTTTTG | |
| | TGATGCTCGTCAGGGGGGCGGAGCCTATGGAAAAACGCCAGCAACGCGGCCTTTTTACGG | |
| | TTCCTGGCCTTTTGCTGGCCTTTTGCTCACATGTTCTTTCCTGCGTTATCCCCTGATTCT | |
| | GTGGATAACCGTATTACCGCCTTTGAGTGAGCTGATACCGCTCGCCGCAGCCGAACGACC | |
| | GAGCGCAGCGAGTCAGTGAGCGAGGAAGCGGAAG | |
| L3 (a.a.) | MDKKYSIGLDIGTNSVGWAVITDEYKVPSKKFKVLGNTDRHSIKKNLIGALLFDSGETAE | 74 |
| | ATRLKRTARRRYTRRKNRICYLQEIFSNEMAKVDDSFFHRLEESFLVEEDKKHERHPIFG | |
| | NIVDEVAYHEKYPTIYHLRKKLVDSTDKADLRLIYLALAHMIKFRGHFLIEGDLNPDNSD | |
| | VDKLFIQLVQTYNQLFEENPINASGVDAKAILSARLSKSRRLENLIAQLPGEKKNGLFGN | |
| | LIALSLGLTPNFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEIFFDQSKNGYA | |
| | GYIDGGASQEEFYKFIKPILEKMDGTEELLVKLNREDLLRKORTFDNGSIPHQIHLGELH | |
| | AILRRQEDFYPFLKDNREKIEKILTFRIPYYVGPLARGNSRFAWMTRKSEETITPWNFEE | |
| | VVDKGASAQSFIERMTNFDKNLPNEKVLPKHSLLYEYFTVYNELTKVKYVTEGMRKPAFL | |
| | SGEQKKAIVDLLFKTNRKVTVKOLKEDYFKKIECFDSVEISGVEDRFNASLGTYHDLLKI | |
| | IKDKDFLDNEENEDILEDIVLTLTLFEDREMIEERLKTYAHLFDDKVMKQLKRRRYTGWG | |
| | RLSRKLINGIRDKQSGKTILDFLKSDGFANRNFMQLIHDDSLTFKEDIQKAQVSGQGDSL | |
| | HEHIANLAGSPAIKKGILQTVKVVDELVKVMGRHKPENIVIEMARENOTTQKGQKNSRER | |
| | MKRIEEGIKELGSQILKEHPVENTQLQNEKLYLYYLONGRDMYVDQELDINRLSDYDVDH | |
| | IVPQSFLKDDSIDNKVLTRSDKNRGKSDNVPSEEVVKKMKNYWROLLNAKLITQRKFDNL | |
| | TKAERGGLSELDKAGFIKRQLVETROITKHVAQILDSRMNTKYDENDKLIREVKVITLKS | |
| | KLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDF | |
| | ATVRKVLSMPQVNIVKKTEVQTGGFSKESILPKRNSDKLIARKKDWDPKKYGGFDSPTVA | |
| | YSVLVVAKVEKGKSMRKGYAMDYKKLKSVKELLGITIMERSSFEKNPIDFLEAKGYKEVK | |
| | KDLIIKLPKYSLFELENGRKRMLASAGELQKGNELALPSKYVNFLYLASHYEKLKGSPED | |
| | NEQKOLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGDGSP | |
| | KKKRKVEDPKKKRKVDSGSETPGTSESATPESAEAGITGTWYNQSGSTFTVTAGADGNLT | |
| | GQYENRAQGTGCONSPYTLTGRYNGTKLEWRVEWNNSTENCHSRTEWRGQYQGGAEARIN | |
| | TQWNLTYEGGSGPATEQGODTFTKVKPSAAS | |

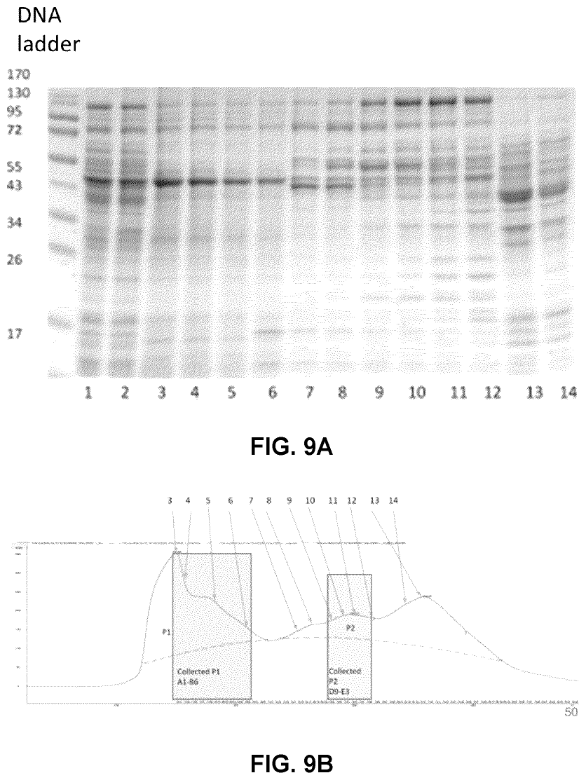

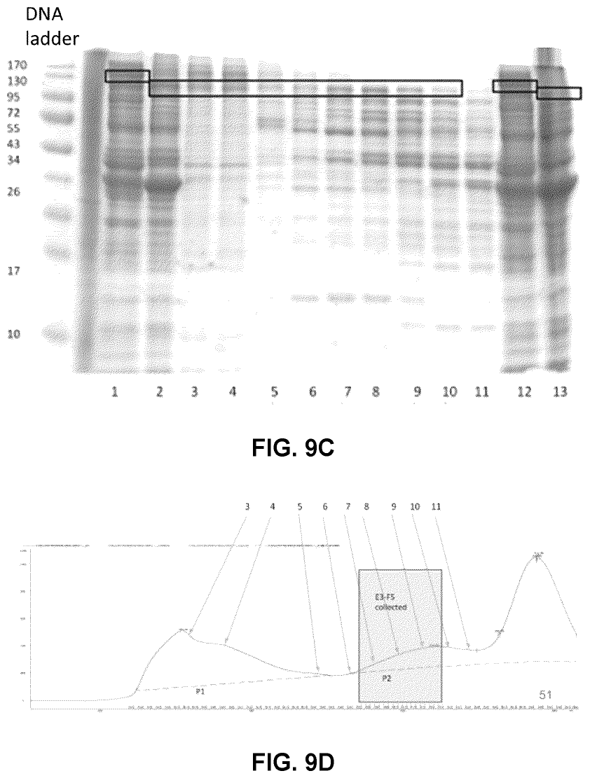

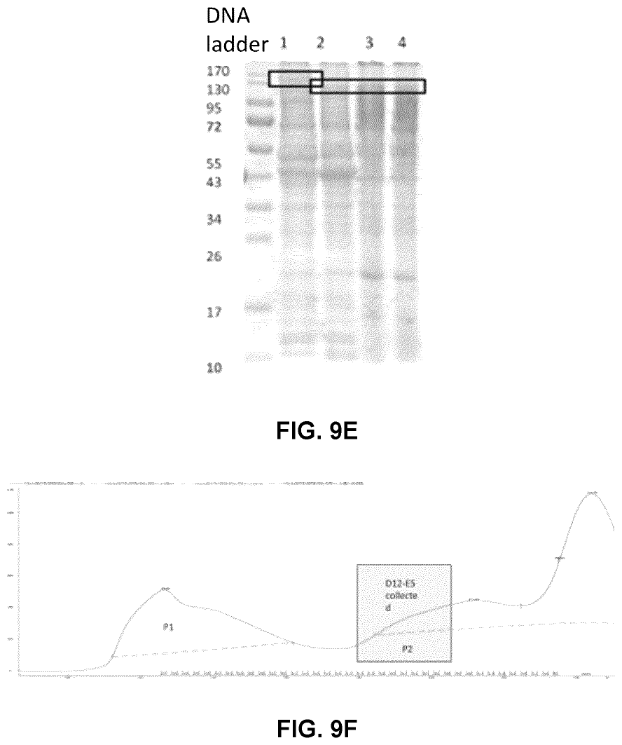

FIGS. 9A and 9B show the expression of Zero, FIGS. 9C and 9D show the expression of L1 and FIGS. 9E and 9F show the expression of L2 and their purification by fast protein liquid chromatography (FLPC). Gel images are used to show the protein content and molecular weight of the fractions collected from FLPC gel filtration. Fractions labeled “1” in FIGS. 9A, 9C, and 9E are the eluted fraction. Fractions labeled “2” are those obtained after TEV protease treatment. Fractions labeled as “3” and above are the fractions obtained from FLPC with Superdex200™ with 0.5 M KCl, 20 mM HEPES in pH 7.5. In brief, proteins were expressed using the common IPTG or autoinduction methods for T7 promoter control expression, in E. coli (De3 BL21 strain). Overnight expression at 18° C. was followed by cell lysis using sonication and gentle detergent lysis, before the first step of his-tag purification using Ni-nitriloacetic acid (NTA) columns. After TEV cleavage of the MBP domain and buffer exchange, the concentrated protein fraction was loaded into the Superdex200™ gel filtration column for size based purification and clean up.

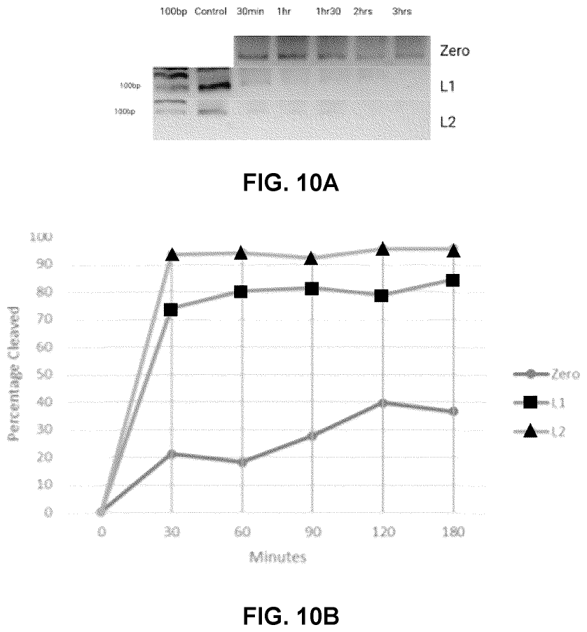

The enzymatic DNA cleaving activity of Zero, L1 and L2 was measured over the course of 3 hours by incubating the enzymes with a 100 bp DNA template at 37° C. To quench the reaction at the different time points (30 mins, 1 h, 1 h30 min, 2 h, and 3 h) 0.5 μL of proteinase K was added to the incubation. The control used was incubating the 100 bp template and adding proteinase K at the appropriate time point. FIGS. 10A and 10B show the cleaving results demonstrating that L1 and L2 are functional nucleases but the cleaving activity of Zero was mitigated (FIG. 10B).

### Protein Expression Testing

The expression vectors, after sequence characterization, were transformed into chemically competent BL21 (DE3). Transformation used either commercial chemically competent B121 based using the calcium chloride method, or homebrew competent cells using the following protocol. The transformation buffer was prepared by first preparing a 1 M calcium chloride solution by dissolving 1.1 g in 10 mL of water, then 1 ml of this solution was transferred to a fresh tube and add 9 ml of distilled water, then it was filter sterilized into a fresh tube which was labeled “transformation buffer”. For improved results, the buffer was prechilled in the fridge for at least an hour before use.

The day before performing the transformation protocol, 10 mL of LB broth was inoculated in a 15 mL tube with BL21 cells, or any other E. coli variety. It was placed into rotating/shaking incubator at 37° C. and was left to grow overnight. 10 ml of fresh LB was inoculated with 100 ul of the overnight solution and left to grow for 2 hours. Pellet cells were recovered by centrifugation at 4500 rpm for 2-3 minutes. The supernatant was discarded and the pellet was resuspended in 1 ml of transformation buffer. The resuspension was transferred to a 1.5 ml tube and re-centrifuged at 12000 rpm for 30 seconds. The supernatant was discarded. 1 mL of transformation buffer was used to resuspend the pellet by gentle pipetting. The centrifugation/resuspension was repeated twice. 100 microliters of transformation buffer was added to the resuspension for high efficiency transformation. 50-400 ng of DNA was added then the mixture was incubated on ice for 30 minutes. The heatblock was preheated to 42° C. and a heat shock of 45 seconds for BL21 and derivatives was performed, or for 30 seconds for T7. The heatshocked solution was immediately chilled in ice for 2 mins. 650 μL of fresh SOC was added, and incubated for 37° C. for 4 hrs with shaking/rotation (particularly for Kan resistant vectors) at 250 rpm. When using DH5, 100 μl was plated on appropriate antibiotic selection plates. When used BL21 (shuffle and derivatives), pellet cells were obtained by centrifugation at 12000 rpm for 10 s. The entire pellet was plated with the addition of 100 μL of media. The pellet was spread using a sterile spreader or innoculation loop. The plates were incubated at 37° C. for 2-3 days until colonies developed.

All vectors and constructs were expressed in BL21 (DE3) in 2× yeast extract trypton (2×YT) or Luria Bertani (LB) media under 0.2-1 mM isopropyl β-D-1-thiogalactopyranoside (IPTG) induction. Initial protein expression tests were conducted in 4 mL culture volumes, prior to scale up purification, as detailed below.

Protein expression vectors were transformed to chemically competent BL21(DE3) E. coli, with a maximum of 100 ng of vector used. After cells were plated upon appropriate antibiotic restrictive plates, single colonies were picked and expression confirmed by growth in 2×YT media in 4 mL culture, induction with 1 mM IPTG at 18° C. for 24 hrs, with rotation at 150 rpm. Once confirmed starter cultures were initiated based on the desired total volume of scale up culture. Scale up cultures were grown at 37° C., until optical density (OD) 600 nm reached (0.6-0.8) and cells were immediately cold shocked to induce chaperone expression, by placing culture vessels in iced water for 15 mins. Once completed induction can be performed with IPTG between 0.2 to 1 mM concentration and incubation completed at 18° C. for 18 to 24 h. Cells were harvested by centrifugation at 4° C. at 5000 rpm. Lysis was performed in 500 mM NaCl, 20 mM tris(hydroxymethyl)aminomethane (TRIS), 10 mM imidazole supplemented with 1 mg/mL of lyzozyme and 0.5% Triton X100. Enzymatic degradation by lyzozyme was performed at 4° C. with shaking for 1 h, with addition of non ethylenediaminetetraacetic acid (EDTA) containing protease inhibitors. After 1 h, Dnase1 and RNase (both at 0.25 mg/ml) and MgCl₂ to 5 mM was added to break down bacterial nucleic acids. Lysis was completed either by freeze thaw or sonication or homogenizer, in order to increase culture volume/pellet mass.

The lysate was clarified by centrifugation at 9000 rpm for 30 mins at 4° C. All following chromatographic steps were performed at 4° C. 2×5 mL HisTrap™ High Performance columns were loaded in parallel with cleared lysate on the column using a peristaltic pump at ˜1.5 mL min-1 overnight at 4° C., to ensure maximum binding. Parallel columns were attached with bound protein to an AKTA™ FPLC liquid chromatography system. Columns were washed with 10 column volumes wash buffer (20 mM Tris-Cl, pH 8.0, 250 mM NaCl, 5 mM imidazole, pH 8.0 at 1.5 mL min⁻¹) until the absorbance nearly reaches the baseline again. Post wash, elution with an imidazole gradient from 0 to 500 mM was performed (elution buffer 20 mM Tris-Cl, pH 8.0, 250 mM NaCl, pH 8.0, 0 to 500 mM imidazole) and collected in 2 mL fractions. Fractions were analysed by using sodium dodecyl sulfate-polyacrylamide gel electrophoresis (SDS-PAGE).

Certain proteins require maltose binding protein (MBP) removal accomplished with 0.5 mg Tobacco Etch Virus (TEV) protease per 50 mg of protein. After which the nuclease sample was diluted to ˜1 mg mL⁻¹ with dialysis buffer (20 mM N-2-hydroxyethylpiperazine-N-2-ethane sulfonic acid (HEPES)—KOH, pH 7.5, 150 mM KCl, 10% (v/v) glycerol, 1 mM dithiothreitol (DTT), 1 mM EDTA) and dialyze the sample in dialysis tubing with a molecular weight cut off (MWCO) of 12-14 kDa against 2 L dialysis buffer at 4° C. overnight. Dialysis buffer (without DTT and glycerol) can be prepared as a 10× stock, but DTT should be added immediately prior to use. The recovered dialyzed sample was centrifuged at 3900 rpm (˜3200×g) for 5 min at 4° C. to remove any precipitate. The TEV protease cleavage was confirmed by using SDS-PAGE.

All proteins obtained (with or without TEV cleavage) were then placed in a size exclusion chromatography (SEC) buffer (20 mM HEPES-KOH, pH 7.5, 500 mM KCl, 1 mM DTT) while concentrating the protein to <1.5 mL volume using a 30,000 MWCO ultracentrifugal filter and filtered through 0.22 μm filter prior to loading in the injection column for gel filtration on a equilibrated HiLoad™ 26/600 Superdex 200 prep grade gel filtration column (GE Healthcare) with the SEC buffer. The concentrated SEC buffer solutions were injected into the column using a 10 mL sample loop. The column was eluted with 320 mL SEC buffer at a flow rate of 1 mL min⁻¹, collecting 2 mL fractions. The peak fractions were analyzed using SDS-PAGE. SDS-PAGE was also performed on fractions that were concentrated. Final samples were exchanged into storage buffers based on the following composition: 25 mM Na phosphate pH 7.25, 300 mM NaCl, 200 mM trehalose (with or without DTT or glycerol depending for short term or long term storage requirements). Proteins were aliquoted and stored at 10 mg/mL concentration.

### Protein Characterization

The proteins Zero, L1, L2, and L3 were purified by a fast performance liquid chromatography (FPLC) fraction collector post gel filtration. The tables listing the fraction identities are presented below (Tables 10 and 11). The proteins Zero, L1, L2, and L3 were also confirmed by mass spectroscopy (spectra not shown). The molecular weights measured for L1 and L2 were respectively 189.3 kDa and 190.3 kDa. 50 μg of each protein (Zero, L1, L2, and L3) was purified and digested using trypsin. The resulting peptide fragments were analyzed using nanoflow HPLC and Orbitrap™ mass spectrometry (quadruple ion trap). The peptide fragments were sequence predicted based on the mass spectrometry.

TABLE 10
fraction identified by the analysis of L1

| | | | | | | | | | | | | |
|---|---|---|---|---|---|---|---|---|---|---|---|---|
| | | | | | | | | | | | | SEQ |
| Peptide | log | log | | | | | | | | | | ID |
| spectrum | (e) | (I) | m + h | delta | Z | zeta | pre | start | sequence | end | post | NO |

| | | | | | | | | | | | | |
|---|---|---|---|---|---|---|---|---|---|---|---|---|
| 18790.1 | −3.2 | 5.83 | 1367.684 | 0.00278 | 2 | 1 | igtn | 15 | SVGWAVITDEYK | 26 | vpsk | 75 |
| 21441.1 | −7 | 5.83 | 2006.055 | 0.00572 | 3 | 1 | hsik | 45 | KNLIGALLFDSGETAEATR | 63 | lkrt | 76 |
| 23432.1 | −2.7 | 5.27 | 2007.039 | 1.003 | 3 | 1 | hsik | 45 | KNLIGALLFDSGETAEATR | 63 | lkrt | 76 |
| 24524.1 | −13.1 | 6.46 | 1877.96 | 0.00041 | 2 | 1 | sikk | 46 | NLIGALLFDSGETAEATR | 63 | lkrt | 77 |
| 24707.1 | −12.9 | 7.14 | 1877.96 | 1.001 | 2 | 1 | sikk | 46 | NLIGALLFDSGETAEATR | 63 | lkrt | 77 |
| 24899.1 | −8.3 | 6.09 | 1877.96 | 0.00066 | 2 | 1 | sikk | 46 | NLIGALLFDSGETAEATR | 63 | lkrt | 77 |
| 24685.1 | −7.1 | 7.01 | 1877.96 | −0.001 | 3 | 1.5 | sikk | 46 | NLIGALLFDSGETAEATR | 63 | lkrt | 77 |
| 25089.1 | −6.8 | 5.76 | 1877.96 | −0.00398 | 2 | 1 | sikk | 46 | NLIGALLFDSGETAEATR | 63 | lkrt | 77 |
| 24878.1 | −5 | 5.69 | 1877.96 | 1.003 | 3 | 1.5 | sikk | 46 | NLIGALLFDSGETAEATR | 63 | lkrt | 77 |
| 25686.1 | −4 | 5.66 | 1877.96 | 0.996 | 2 | 1 | sikk | 46 | NLIGALLFDSGETAEATR | 63 | lkrt | 77 |
| 25477.1 | −3.8 | 5.46 | 1877.96 | 1.005 | 2 | 1 | sikk | 46 | NLIGALLFDSGETAEATR | 63 | lkrt | 77 |
| 25285.1 | −3.6 | 5.58 | 1877.96 | 0.00176 | 2 | 1 | sikk | 46 | NLIGALLFDSGETAEATR | 63 | lkrt | 77 |
| 24504.1 | −3 | 5.4 | 1877.96 | 1.002 | 3 | 1.5 | sikk | 46 | NLIGALLFDSGETAEATR | 63 | lkrt | 77 |
| 21077.1 | −9.9 | 6.38 | 1763.917 | 0.00074 | 2 | 1 | ikkn | 47 | LIGALLFDSGETAEATR | 63 | lkrt | 78 |
| 21061.1 | −7.4 | 6.14 | 1763.917 | 1.004 | 2 | 1 | ikkn | 47 | LIGALLFDSGETAEATR | 63 | lkrt | 78 |
| 21106.1 | −5.1 | 5.86 | 1763.917 | −0.00092 | 3 | 1.5 | ikkn | 47 | LIGALLFDSGETAEATR | 63 | lkrt | 78 |
| 18096.1 | −4.6 | 5.81 | 1650.833 | −0.00248 | 2 | 1 | kknl | 48 | IGALLFDSGETAEATR | 63 | lkrt | 79 |
| 15873.1 | −6 | 6 | 1480.728 | 0.00246 | 2 | 1 | nlig | 50 | ALLFDSGETAEATR | 63 | lkrt | 80 |
| 14989.1 | −2.6 | 5.59 | 1409.691 | 1.002 | 2 | 1 | liga | 51 | LLFDSGETAEATR | 63 | lkrt | 81 |
| 11243.1 | −4.1 | 5.99 | 1296.607 | 0.00084 | 2 | 1 | igal | 52 | LFDSGETAEATR | 63 | lkrt | 82 |
| 8144.1 | −4.8 | 5.87 | 1183.523 | 0.00055 | 2 | 1 | gall | 53 | FDSGETAEATR | 63 | lkrt | 83 |
| 21031.1 | −5.1 | 6.46 | 1761.819 | −0.00009 | 2 | 1 | rknr | 79 | ICYLQEIFSNEMAK | 92 | vdds | 84 |
| 21495.1 | −2.5 | 5.99 | 1777.814 | 0.00303 | 3 | 1.5 | rknr | 79 | ICYLQEIFSNEMAK | 92 | vdds | 84 |
| 20879.1 | −2.4 | 5.96 | 1761.819 | 0.00076 | 3 | 1.5 | rknr | 79 | ICYLQEIFSNEMAK | 92 | vdds | 84 |
| 12577.1 | −5.3 | 6.07 | 1022.469 | 0.001 | 2 | 0.667 | emak | 93 | VDDSFFHR | 100 | lees | 85 |
| 12377.1 | −4.8 | 5.86 | 1022.469 | 0.0007 | 2 | 0.667 | emak | 93 | VDDSFFHR | 100 | lees | 85 |
| 16821.1 | −4.3 | 5.58 | 1135.553 | 0.00251 | 2 | 0.667 | emak | 93 | VDDSFFHRL | 101 | eesf | 86 |
| 15598.1 | −4.9 | 6.53 | 1337.647 | −0.0002 | 2 | 1 | ffhr | 101 | LEESFLVEEDK | 111 | kher | 87 |
| 15805.1 | −3.7 | 6.19 | 1337.647 | −0.00057 | 2 | 1 | ffhr | 101 | LEESFLVEEDK | 111 | kher | 87 |
| 13638.1 | −7.7 | 6.09 | 1465.742 | 1.003 | 2 | 0.667 | ffhr | 101 | LEESFLVEEDKK | 112 | herh | 88 |
| 13845.1 | −5.3 | 5.84 | 1465.742 | 0.00262 | 2 | 0.667 | ffhr | 101 | LEESFLVEEDKK | 112 | herh | 88 |
| 13630.1 | −2.6 | 6.03 | 1465.742 | 0.00053 | 3 | 1 | ffhr | 101 | LEESFLVEEDKK | 112 | herh | 88 |
| 11355.1 | −11.9 | 7.01 | 1887.945 | −0.00006 | 3 | 0.6 | ffhr | 101 | LEESFLVEEDKKHER | 115 | hpif | 89 |
| 11582.1 | −5.2 | 5.45 | 1887.945 | 0.00195 | 3 | 0.6 | ffhr | 101 | LEESFLVEEDKKHER | 115 | hpif | 89 |
| 17973.1 | −8.4 | 6.1 | 1868.918 | 0.018 | 2 | 0.5 | kher | 116 | HPIFGNIVDEVAYHEK | 131 | ypti | 90 |
| 18314.1 | −6 | 6.23 | 1867.934 | 1.005 | 3 | 0.75 | kher | 116 | HPIFGNIVDEVAYHEK | 131 | ypti | 90 |
| 19173.1 | −4.7 | 5.58 | 1868.918 | 1.007 | 3 | 0.75 | kher | 116 | HPIFGNIVDEVAYHEK | 131 | ypti | 90 |
| 18105.1 | −4.6 | 7.3 | 1867.934 | 1.001 | 3 | 0.75 | kher | 116 | HPIFGNIVDEVAYHEK | 131 | ypti | 90 |
| 18524.1 | −4.2 | 5.67 | 1867.934 | 1.005 | 3 | 0.75 | kher | 116 | HPIFGNIVDEVAYHEK | 131 | ypti | 90 |
| 18741.1 | −3.4 | 5.5 | 1867.934 | −0.004 | 3 | 0.75 | kher | 116 | HPIFGNIVDEVAYHEK | 131 | ypti | 90 |
| 20133.1 | −3.4 | 5.86 | 1730.875 | 1.006 | 3 | 1 | herh | 117 | PIFGNIVDEVAYHEK | 131 | ypti | 91 |
| 18496.1 | −3.8 | 5.48 | 1633.822 | 0.00258 | 3 | 1 | erhp | 118 | IFGNIVDEVAYHEK | 131 | ypti | 92 |
| 9245.1 | −4.3 | 6.01 | 1202.605 | −0.00222 | 2 | 0.667 | ifgn | 122 | IVDEVAYHEK | 131 | ypti | 93 |
| 13116.1 | −4.7 | 6.21 | 1062.573 | −0.00065 | 2 | 0.667 | yhek | 132 | YPTIYHLR | 139 | kklv | 94 |
| 13325.1 | −3.7 | 6.71 | 1062.573 | 0.00081 | 2 | 0.667 | yhek | 132 | YPTIYHLR | 139 | kklv | 94 |
| 13534.1 | −2.6 | 6.61 | 1062.573 | −0.00065 | 2 | 0.667 | yhek | 132 | YPTIYHLR | 139 | kklv | 94 |
| 8278.1 | −6.2 | 6.51 | 1360.743 | −0.00075 | 3 | 0.75 | hlrk | 141 | KLVDSTDKADLR | 152 | liyl | 95 |
| 8268.1 | −2.2 | 5.55 | 1360.743 | 0.00088 | 2 | 0.5 | hlrk | 141 | KLVDSTDKADLR | 152 | liyl | 95 |
| 8488.1 | −5.1 | 5.97 | 1232.648 | 0.00185 | 2 | 0.667 | lrkk | 142 | LVDSTDKADLR | 152 | liyl | 96 |
| 8499.1 | −4.6 | 6.67 | 1232.648 | −0.00033 | 3 | 1 | lrkk | 142 | LVDSTDKADLR | 152 | liyl | 96 |
| 8693.1 | −4.2 | 5.47 | 1232.648 | 0.00148 | 2 | 0.667 | lrkk | 142 | LVDSTDKADLR | 152 | liyl | 96 |
| 8705.1 | −2.1 | 5.69 | 1232.648 | 0.00068 | 3 | 1 | lrkk | 142 | LVDSTDKADLR | 152 | liyl | 96 |
| 18128.1 | −7.4 | 6.99 | 1301.765 | 0.00008 | 2 | 0.667 | adlr | 153 | LIYLALAHMIK | 163 | frgh | 97 |
| 19075.1 | −6 | 6.25 | 1317.76 | 0.00064 | 2 | 0.667 | adlr | 153 | LIYLALAHMIK | 163 | frgh | 97 |
| 17922.1 | −2.8 | 5.82 | 1301.765 | 0.00179 | 2 | 0.667 | adlr | 153 | LIYLALAHMIK | 163 | frgh | 97 |
| 18127.1 | −2.6 | 7.26 | 1301.765 | 0.00006 | 3 | 1 | adlr | 153 | LIYLALAHMIK | 163 | frgh | 97 |
| 17921.1 | −2.4 | 6.24 | 1301.765 | 0.00199 | 3 | 1 | adlr | 153 | LIYLALAHMIK | 163 | frgh | 97 |
| 21110.1 | −2.1 | 6.5 | 1285.77 | −0.00267 | 3 | 1 | adlr | 153 | LIYLALAHMIK | 163 | frgh | 97 |
| 16054.1 | −8.7 | 5.82 | 1984.925 | 1.008 | 2 | 0.667 | ikfr | 166 | GHFLIEGDLNPDNSDVDK | 183 | lfiq | 98 |
| 16234.1 | −6.1 | 6.31 | 1984.925 | 1.005 | 3 | 1 | ikfr | 166 | GHFLIEGDLNPDNSD | 183 | lfiq | 98 |
| | | | | | | | | | VDK | | | |
| 16028.1 | −3.8 | 5.96 | 1984.925 | 1.003 | 3 | 1 | ikfr | 166 | GHFLIEGDLNPDNSD | 183 | lfiq | 98 |
| | | | | | | | | | VDK | | | |
| 16260.1 | −3 | 5.53 | 1984.925 | 1.011 | 2 | 0.667 | ikfr | 166 | GHFLIEGDLNPDNSD | 183 | lfiq | 98 |
| | | | | | | | | | VDK | | | |
| 16671.1 | −2.1 | 5.42 | 1984.925 | 1.994 | 3 | 1 | ikfr | 166 | GHFLIEGDLNPDNSD | 183 | lfiq | 98 |
| | | | | | | | | | VDK | | | |
| 17489.1 | −2.1 | 5.48 | 1790.844 | −0.00086 | 2 | 1 | frgh | 168 | FLIEGDLNPDNSDVD | 183 | lfiq | 99 |
| | | | | | | | | | K | | | |
| 20110.1 | −5.8 | 5.61 | 2450.22 | 1.007 | 3 | 1 | lfiq | 188 | LVQTYNQLFEENPINA | 209 | ails | 100 |
| | | | | | | | | | SGVDAK | | | |
| 16683.1 | −5.3 | 5.53 | 1845.898 | 0.00454 | 2 | 1 | vqty | 193 | NQLFEENPINASGVD | 209 | ails | 101 |
| | | | | | | | | | AK | | | |
| 16745.1 | −2.2 | 5.47 | 1845.898 | 0.999 | 3 | 1 | vqty | 193 | NQLFEENPINASGVD | 209 | ails | 101 |
| | | | | | | | | | AK | | | |
| 16443.1 | −11.1 | 6.48 | 1731.855 | 0.00047 | 2 | 1 | qtyn | 194 | QLFEENPINASGVDA | 209 | ails | 102 |
| | | | | | | | | | K | | | |
| 17102.1 | −7 | 6.9 | 1480.848 | 0.00004 | 3 | 1 | sksr | 221 | RLENLIAQLPGEK | 233 | kngl | 103 |
| 16897.1 | −6.6 | 6.72 | 1480.848 | 0.00206 | 3 | 1 | sksr | 221 | RLENLIAQLPGEK | 233 | kngl | 103 |
| 16896.1 | −5.2 | 6.42 | 1480.848 | 0.00231 | 2 | 0.667 | sksr | 221 | RLENLIAQLPGEK | 233 | kngl | 103 |
| 17729.1 | −5.2 | 6.22 | 1481.832 | −0.00015 | 3 | 1 | sksr | 221 | RLENLIAQLPGEK | 233 | kngl | 103 |
| 17311.1 | −4.8 | 6.15 | 1480.848 | 0.00114 | 3 | 1 | sksr | 221 | RLENLIAQLPGEK | 233 | kngl | 103 |
| 17091.1 | −3.9 | 6.54 | 1480.848 | −0.00025 | 2 | 0.667 | sksr | 221 | RLENLIAQLPGEK | 233 | kngl | 103 |
| 17520.1 | −2.5 | 5.81 | 1480.848 | 0.00233 | 3 | 1 | sksr | 221 | RLENLIAQLPGEK | 233 | kngl | 103 |
| 15455.1 | −8.4 | 6.86 | 1608.943 | −0.00019 | 3 | 0.75 | sksr | 221 | RLENLIAQLPGEKK | 234 | nglf | 104 |
| 18183.1 | −7.2 | 6.3 | 1324.747 | −0.00034 | 2 | 1 | ksrr | 222 | LENLIAQLPGEK | 233 | kngl | 105 |
| 18043.1 | −3.8 | 6.14 | 1324.747 | −0.00102 | 3 | 1 | ksrr | 222 | LENLIAQLPGEK | 233 | kngl | 105 |
| 17984.1 | −3.3 | 6.19 | 1324.747 | −0.0007 | 2 | 1 | ksrr | 222 | LENLIAQLPGEK | 233 | kngl | 105 |
| 18057.1 | −3.1 | 5.68 | 1324.747 | −0.00099 | 1 | 0.5 | ksrr | 222 | LENLIAQLPGEK | 233 | kngl | 105 |
| 18755.1 | −2.8 | 5.86 | 1325.731 | −0.00022 | 2 | 1 | ksrr | 222 | LENLIAQLPGEK | 233 | kngl | 105 |
| 18433.1 | −2.1 | 5.73 | 1325.731 | 0.011 | 2 | 1 | ksrr | 222 | LENLIAQLPGEK | 233 | kngl | 105 |
| 16259.1 | −3.4 | 5.98 | 1452.842 | 0.00321 | 2 | 0.667 | ksrr | 222 | LENLIAQLPGEKK | 234 | nglf | 106 |
| 22403.1 | −2.3 | 5.45 | 1386.836 | 0.00158 | 2 | 1 | Ifgn | 241 | LIALSLGLTPNFK | 253 | snfd | 107 |
| 12459.1 | −3 | 5.87 | 1384.674 | 1.007 | 2 | 0.667 | Itpn | 252 | FKSNFDLAEDAK | 263 | lqls | 108 |
| 11618.1 | −2.8 | 5.46 | 1109.511 | −0.00088 | 2 | 1 | pnfk | 254 | SNFDLAEDAK | 263 | lqls | 109 |
| 12508.1 | −2.5 | 7.47 | 1109.511 | 0.00254 | 2 | 1 | pnfk | 254 | SNFDLAEDAK | 263 | lqls | 109 |
| 12710.1 | −2.4 | 5.98 | 1109.511 | −0.00052 | 2 | 1 | pnfk | 254 | SNFDLAEDAK | 263 | lqls | 109 |
| 24999.1 | −4.1 | 5.64 | 1712.991 | 0.034 | 2 | 0.667 | dlfl | 292 | AAKNLSDAILLSDILR | 307 | vnte | 110 |
| 25323.1 | −8.2 | 6.54 | 1442.821 | 0.00136 | 2 | 1 | laak | 295 | NLSDAILLSDILR | 307 | vnte | 111 |
| 26535.1 | −7.3 | 6.26 | 1443.805 | −0.00023 | 2 | 1 | laak | 295 | NLSDAILLSDILR | 307 | vnte | 111 |
| 25125.1 | −5.3 | 7.72 | 1442.82 | −0.00047 | 2 | 1 | laak | 295 | NLSDAILLSDILR | 307 | vnte | 111 |
| 25520.1 | −4.7 | 6.11 | 1442.821 | 0.00039 | 2 | 1 | laak | 295 | NLSDAILLSDILR | 307 | vnte | 111 |
| 25728.1 | −4.4 | 5.85 | 1442.821 | 0.00039 | 2 | 1 | laak | 295 | NLSDAILLSDILR | 307 | vnte | 111 |
| 24933.1 | −4.2 | 5.84 | 1442.821 | 1.005 | 2 | 1 | laak | 295 | NLSDAILLSDILR | 307 | vnte | 111 |
| 25931.1 | −4.1 | 5.69 | 1442.82 | −0.00059 | 2 | 1 | laak | 295 | NLSDAILLSDILR | 307 | vnte | 111 |
| 26130.1 | −3.3 | 5.61 | 1442.82 | 0.00014 | 2 | 1 | laak | 295 | NLSDAILLSDILR | 307 | vnte | 111 |
| 26334.1 | −2.6 | 5.48 | 1442.821 | −0.00022 | 2 | 1 | laak | 295 | NLSDAILLSDILR | 307 | vnte | 111 |
| 25171.1 | −2.4 | 6.19 | 1442.821 | 0.0005 | 3 | 1.5 | laak | 295 | NLSDAILLSDILR | 307 | vnte | 111 |
| 13074.1 | −3.4 | 5.38 | 1718.936 | 1.005 | 2 | 0.667 | dilr | 308 | VNTEITKAPLSASMIK | 323 | ryde | 112 |
| 10752.1 | −4.7 | 5.56 | 949.5023 | −0.00011 | 2 | 1 | eitk | 315 | APLSASMIK | 323 | ryde | 113 |
| 10137.1 | −4 | 5.99 | 933.5074 | 0.00091 | 2 | 1 | eit | 315 | APLSASMIK | 323 | ryde | 113 |
| 99301.1. | −2.9 | 5.78 | 933.5074 | 0.00085 | 2 | 1 | eitk | 315 | APLSASMIK | 323 | ryde | 113 |
| 11038.1 | −5.7 | 6.62 | 1667.85 | 0.00094 | 3 | 0.6 | smik | 324 | RYDEHHQDLTLLK | 336 | alvr | 114 |
| 12108.1 | −5 | 7.13 | 1511.749 | 0.00097 | 3 | 0.75 | mikr | 325 | YDEHHQDLTLLK | 336 | alvr | 115 |
| 13869.1 | −6 | 6.68 | 1304.652 | 0.00173 | 2 | 0.667 | lpek | 347 | YKEIFFDQSK | 356 | ngya | 116 |
| 14076.1 | −4.4 | 5.89 | 1304.652 | 0.00161 | 2 | 0.667 | lpek | 347 | YKEIFFDQSK | 356 | ngya | 116 |
| 14362.1 | −3.4 | 7.98 | 1013.494 | 0.00158 | 2 | 1 | ekyk | 349 | EIFFDQSK | 356 | ngya | 117 |
| 17817.1 | −14.5 | 5.79 | 1969.845 | 0.00244 | 2 | 1 | dqsk | 357 | NGYAGYIDGGASQEE | 374 | fikp | 118 |
| | | | | | | | | | FYK | | | |
| 17608.1 | −14.3 | 7.3 | 1969.845 | 0.00183 | 2 | 1 | dqsk | 357 | NGYAGYIDGGASQEE | 374 | fikp | 118 |
| | | | | | | | | | FYK | | | |
| 16982.1 | −12.9 | 6.39 | 1969.845 | 0.00073 | 2 | 1 | dqsk | 357 | NGYAGYIDGGASQEE | 374 | fikp | 118 |
| | | | | | | | | | FYK | | | |
| 16553.1 | −10.1 | 6.18 | 1968.86 | 1.003 | 2 | 1 | dqsk | 357 | NGYAGYIDGGASQEE | 374 | fikp | 118 |
| | | | | | | | | | FYK | | | |
| 16379.1 | −6 | 5.88 | 1968.861 | 1.007 | 3 | 1.5 | dqsk | 357 | NGYAGYIDGGASQEE | 374 | fikp | 118 |
| | | | | | | | | | FYK | | | |
| 17589.1 | −5.7 | 5.77 | 1969.845 | 0.00285 | 3 | 1.5 | dqsk | 357 | NGYAGYIDGGASQEE | 374 | fikp | 118 |
| | | | | | | | | | FYK | | | |
| 16102.1 | −3.8 | 5.53 | 1968.86 | 1.001 | 2 | 1 | dqsk | 357 | NGYAGYIDGGASQEE | 374 | fikp | 118 |
| | | | | | | | | | FYK | | | |
| 17179.1 | −3 | 5.52 | 1968.86 | 0.987 | 2 | 1 | dqsk | 357 | NGYAGYIDGGASQEE | 374 | fikp | 118 |
| | | | | | | | | | FYK | | | |
| 16921.1 | −2.7 | 5.36 | 1969.845 | 1.001 | 3 | 1.5 | dqsk | 357 | NGYAGYIDGGASQEE | 374 | fikp | 118 |
| | | | | | | | | | FYK | | | |
| 9935.1 | −2.6 | 5.27 | 1115.5 | 0.00077 | 2 | 1 | gyid | 365 | GGASQEEFYK | 374 | fikp | 119 |
| 12399.1 | −7.2 | 6.44 | 987.6237 | −0.00002 | 2 | 0.667 | efyk | 375 | FIKPILEK | 382 | mdgt | 120 |
| 16489.1 | −4.7 | 6.21 | 2119.172 | 1.004 | 3 | 0.75 | efyk | 375 | FIKPILEKMDGTEELL | 392 | lnre | 121 |
| | | | | | | | | | VK | | | |
| 13371.1 | −5.6 | 6.74 | 1166.56 | 0.00003 | 2 | 1 | ilek | 383 | MDGTEELLVK | 392 | lnre | 122 |
| 12992.1 | −5 | 6.54 | 1150.566 | 0.0013 | 2 | 1 | ilek | 383 | MDGTEELLVK | 392 | lnre | 122 |
| 12788.1 | −4.5 | 6.93 | 1150.566 | −0.00017 | 2 | 1 | ilek | 383 | MDGTEELLVK | 392 | lnre | 122 |
| 13769.1 | −4.2 | 5.36 | 1150.566 | 0.00105 | 2 | 1 | ilek | 383 | MDGTEELLVK | 392 | lnre | 122 |
| 13347.1 | −3.3 | 5.64 | 1150.566 | 0.00093 | 2 | 1 | ilek | 383 | MDGTEELLVK | 392 | lnre | 122 |
| 18975.1 | −5.4 | 5.73 | 2369.236 | 1.006 | 3 | 0.6 | rkqr | 404 | TFDNGSIPHQIHLGEL | 424 | rqed | 123 |
| | | | | | | | | | HAILR | | | |
| 18768.1 | −5.2 | 5.84 | 2370.22 | 1.021 | 3 | 0.6 | rkqr | 404 | TFDNGSIPHQIHLGEL | 424 | rqed | 123 |
| | | | | | | | | | HAILR | | | |
| 18323.1 | −2.8 | 5.85 | 2370.22 | 0.04 | 3 | 0.6 | rkqr | 404 | TFDNGSIPHQIHLGEL | 424 | rqed | 123 |
| | | | | | | | | | HAILR | | | |
| 18370.1 | −2.4 | 5.37 | 2368.252 | 1.001 | 2 | 0.4 | rkqr | 404 | TFDNGSIPHQIHLGEL | 424 | rqed | 123 |
| | | | | | | | | | HAILR | | | |
| 18370.2 | −2.4 | 5.37 | 2369.236 | 0.017 | 2 | 0.4 | rkqr | 404 | TFDNGSIPHQIHLGEL | 424 | rqed | 123 |
| | | | | | | | | | HAILR | | | |
| 16314.1 | −2.1 | 5.8 | 1399.817 | 0.00311 | 3 | 0.75 | siph | 413 | QIHLGELHAILR | 424 | rqed | 124 |
| 18101.1 | −2.4 | 6.17 | 1101.5 | 0.00043 | 2 | 1 | ailr | 425 | RQEDFYPF | 432 | lkdn | 125 |
| 17060.1 | −5 | 7.44 | 1342.679 | 0 | 2 | 0.667 | ailr | 425 | RQEDFYPFLK | 434 | dnre | 126 |
| 17476.1 | −3.9 | 5.66 | 1342.679 | 0.00109 | 2 | 0.667 | ailr | 425 | RQEDFYPFLK | 434 | dnre | 126 |
| 17267.1 | −3.8 | 6.43 | 1342.679 | 0.0017 | 2 | 0.667 | ailr | 452 | RQEDFYPFLK | 434 | dnre | 126 |
| 17001.1 | −3.8 | 6.31 | 1342.679 | 1.006 | 3 | 1 | ailr | 425 | RQEDFYPFLK | 434 | dnre | 126 |
| 17410.1 | −3.6 | 6.35 | 1342.679 | −0.0006 | 3 | 1 | ailr | 425 | RQEDFYPFLK | 434 | dnre | 126 |
| 17201.1 | −3 | 7.45 | 1342.679 | 0.00068 | 3 | 1 | ailr | 425 | RQEDFYPFLK | 434 | dnre | 126 |
| 15095.1 | −6.5 | 6.14 | 1727.85 | 0.00185 | 3 | 0.75 | ailr | 425 | RQEDFYPFLKDNR | 437 | ekie | 127 |
| 19427.1 | −3 | 6.08 | 1186.578 | 0.0004 | 2 | 1 | ilrr | 426 | QEDFYPFLK | 434 | dnre | 128 |
| 19218.1 | −3 | 7.07 | 1186.578 | 0.00064 | 2 | 1 | ilrr | 426 | QEDFYPFLK | 434 | dnre | 128 |
| 16828.1 | −4.5 | 5.94 | 1571.749 | 0.00226 | 3 | 1 | ilrr | 427 | QEDFYPFLKDNR | 436 | ekie | 129 |
| 16907.1 | −5.3 | 6.1 | 1148.646 | 0.00138 | 2 | 1 | ltfr | 448 | IPYYVGPLAR | 457 | gnsr | 130 |
| 17322.1 | −4.4 | 6.41 | 1148.646 | 0.00223 | 2 | 1 | ltfr | 448 | IPYYVGPLAR | 457 | gnsr | 130 |
| 17113.1 | −4.4 | 7.76 | 1148.646 | 0.00028 | 2 | 1 | ltfr | 448 | IPYYVGPLAR | 457 | gnsr | 130 |
| 17619.1 | −3.2 | 6 | 1148.646 | 0.00126 | 2 | 1 | ltfr | 448 | IPYYVGPLAR | 457 | gnsr | 130 |
| 17828.1 | −3 | 5.76 | 1148.646 | 0.00101 | 2 | 1 | ltfr | 448 | IPYYVGPLAR | 457 | gnsr | 130 |
| 18735.1 | −2.6 | 5.28 | 1148.646 | 1.002 | 2 | 1 | ltfr | 448 | IPYYVGPLAR | 457 | gnsr | 130 |
| 18032.1 | −2.3 | 5.55 | 1148.646 | 0.00107 | 2 | 1 | ltfr | 448 | PYYVGPLAR | 457 | gnsr | 130 |
| 13517.1 | −2.4 | 5.47 | 938.5094 | 0.00326 | 2 | 1 | frip | 450 | YYVGPLAR | 457 | gnsr | 131 |
| 7984.1 | −2 | 5.74 | 843.3818 | −0.00008 | 2 | 1 | gnsr | 462 | FAWMTR | 467 | ksee | 132 |
| 20120.1 | −12.3 | 6.25 | 2051.981 | 0.02 | 2 | 0.667 | wmtr | 468 | KSEETITPWNFEEVV | 484 | gasa | 133 |
| | | | | | | | | | DK | | | |
| 18446.1 | −5.7 | 6.58 | 2082.987 | 1.004 | 3 | 1 | wmtr | 468 | KSEETITPWNFEEVV | 484 | gasa | 133 |
| | | | | | | | | | DK | | | |
| 20109.1 | −4.9 | 7.01 | 2050.997 | 1.004 | 3 | 1 | wmtr | 468 | KSEETITPWNFEEVV | 484 | gasa | 133 |
| | | | | | | | | | DK | | | |
| 18864.1 | −3.6 | 6.03 | 2082.987 | 0.999 | 3 | 1 | wmtr | 468 | KSEETITPWNFEEVV | 484 | gasa | 133 |
| | | | | | | | | | DK | | | |
| 20228.1 | −3.2 | 5.39 | 2066.992 | 1.012 | 3 | 1 | wmtr | 468 | KSEETITPWNFEEVV | 484 | gasa | 133 |
| | | | | | | | | | DK | | | |
| 18004.1 | −3 | 6.39 | 2066.992 | 1.002 | 3 | 1 | wmtr | 468 | KSEETITPWNFEEVV | 484 | gasa | 133 |
| | | | | | | | | | DK | | | |
| 20131.1 | −2.6 | 6.06 | 2082.987 | 1.005 | 3 | 1 | wmtr | 468 | KSEETITPWNFEEVV | 484 | gasa | 133 |
| | | | | | | | | | DK | | | |
| 16515.1 | −2.5 | 5.47 | 2066.992 | 1.002 | 3 | 1 | wmtr | 468 | KSEETITPWNFEEVV | 484 | gasa | 133 |
| | | | | | | | | | DK | | | |
| 19236.1 | −2 | 5.55 | 2066.992 | −0.00087 | 3 | 1 | wmtr | 468 | KSEETITPWNFEEVV | 484 | gasa | 133 |
| | | | | | | | | | DK | | | |
| 22157.1 | −3.4 | 5.61 | 1922.902 | 0.00278 | 2 | 1 | mtrk | 469 | SEETITPWNFEEVVD | 484 | gasa | 134 |
| | | | | | | | | | K | | | |
| 20207.1 | −3.4 | 6.19 | 1954.892 | 1.002 | 2 | 1 | mtrk | 469 | SEETITPWNFEEVVD | 484 | gasa | 134 |
| | | | | | | | | | K | | | |
| 18250.1 | −3.3 | 5.78 | 1938.897 | 0.00156 | 3 | 1 | mtrk | 469 | SEETITPWNFEEVVD | 484 | gasa | 134 |
| | | | | | | | | | K | | | |
| 20316.1 | −5.2 | 6.08 | 2050.96 | 0.036 | 3 | 1 | mtrk | 469 | SEETITPWNFEEVVD | 486 | saqs | 135 |
| | | | | | | | | | KGA | | | |
| 17432.1 | −5.7 | 6.24 | 2025.988 | 0.999 | 3 | 1 | itpw | 477 | NFEEVVDKGASAQSF | 494 | mtnf | 136 |
| | | | | | | | | | IER | | | |
| 17465.1 | −3.2 | 5.4 | 2025.988 | 1 | 2 | 0.667 | itpw | 477 | NFEEVVDKGASAQSF | 494 | mtnf | 136 |
| | | | | | | | | | IER | | | |
| 11939.1 | −4.3 | 6.31 | 1065.532 | 0.00018 | 2 | 1 | vvdk | 485 | GASAQSFIER | 494 | mtnf | 137 |
| 11739.1 | −3.6 | 5.88 | 1065.532 | 0.00274 | 2 | 1 | vvdk | 485 | GASAQSFIER | 494 | mtnf | 137 |
| 10788.1 | −3.7 | 5.82 | 937.4738 | 0.00053 | 2 | 1 | dkga | 487 | SAQSFIER | 494 | mtnf | 138 |
| 10294.1 | −8.6 | 5.88 | 1482.689 | 0.00202 | 2 | 0.667 | fier | 495 | MTNFDKNLPNEK | 506 | vlpk | 139 |
| 9745.1 | −4.1 | 5.71 | 1466.694 | 0.00107 | 3 | 1 | fiel | 495 | MTNFDKNLPNEK | 506 | vlpk | 139 |
| 9942.1 | −3.5 | 5.51 | 1466.694 | 0.0012 | 2 | 0.667 | fier | 495 | MTNFDKNLPNEK | 506 | vlpk | 139 |
| 9743.1 | −3.2 | 5.31 | 1466.694 | 0.00108 | 2 | 0.667 | fier | 495 | MTNFDKNLPNEK | 506 | vlpk | 139 |
| 10240.1 | −3.1 | 5.69 | 1482.689 | −0.00004 | 3 | 1 | fier | 495 | MTNFDKNLPNEK | 506 | vlpk | 139 |
| 11010.1 | −2.7 | 6.08 | 1450.699 | 0.00099 | 3 | 1 | fier | 495 | MTNFDKNLPNEK | 506 | vlpk | 139 |
| 9941.1 | −2.5 | 5.83 | 1466.694 | 0.00134 | 3 | 1 | fier | 495 | MTNFDKNLPNEK | 506 | vlpk | 139 |
| 25193.1 | −4.1 | 5.36 | 2020.006 | 1.003 | 3 | 1 | vlpk | 511 | HSLLYEYFTVYNELTK | 526 | vkyv | 140 |
| 25390.1 | −3.4 | 5.7 | 2020.006 | 1.004 | 3 | 1 | vlpk | 511 | HSLLYEYFTVYNELTK | 526 | vkyv | 140 |
| 6322.1 | −3.3 | 7.36 | 871.3978 | 0.00017 | 2 | 1 | tkvk | 529 | YVTEGMR | 535 | kpaf | 141 |
| 8398.1 | −2.4 | 6.23 | 855.4029 | 0.00052 | 2 | 1 | tkvk | 529 | YVTEGMR | 535 | kpaf | 141 |
| 9342.1 | −6 | 6.91 | 1104.605 | 0.00049 | 2 | 0.667 | egmr | 536 | KPAFLSGEQK | 545 | kaiv | 142 |
| 9148.1 | −6 | 6.05 | 1104.605 | −0.00109 | 2 | 0.667 | egmr | 536 | KPAFLSGEQK | 545 | kaiv | 142 |
| 9695.1 | −3.2 | 5.39 | 1105.589 | −0.00122 | 2 | 0.667 | egmr | 536 | KPAFLSGEQK | 545 | kaiv | 142 |
| 10908.1 | −3.3 | 5.5 | 976.5098 | 0.00067 | 2 | 1 | gmrk | 537 | PAFLSGEQK | 545 | kaiv | 143 |
| 19007.1 | −4.8 | 6.02 | 1046.661 | −0.00002 | 2 | 0.667 | geqk | 546 | KAIVDLLFK | 554 | tnrk | 144 |
| 14692.1 | −4.4 | 6.4 | 1053.525 | 0.00104 | 2 | 0.667 | vtvk | 563 | QLKEDYFK | 570 | kiec | 145 |
| 10895.1 | −2.6 | 6.66 | 1070.552 | 0.00147 | 2 | 0.667 | vtvk | 563 | QLKEDYFK | 570 | kiec | 145 |
| 1550.12 | −15 | 5.8 | 1882.885 | −0.00067 | 2 | 0.667 | dyfk | 571 | KIECFDSVEISGVEDR | 586 | fnas | 146 |
| 15728.1 | −7.8 | 5.99 | 1882.885 | 0.00194 | 3 | 1 | dyfk | 571 | KIECFDSVEISGVEDR | 586 | fnas | 146 |
| 15519.1 | −6.9 | 6.41 | 1882.885 | 0.00066 | 3 | 1 | dyfk | 571 | KIECFDSVEISGVEDR | 586 | fnas | 146 |
| 15217.1 | −5.1 | 5.65 | 1882.885 | 0.00396 | 3 | 1 | dyfk | 571 | KIECFDSVEISGVEDR | 586 | fnas | 146 |
| 16082.1 | −2.2 | 5.24 | 1882.885 | 1.001 | 3 | 1 | dyfk | 571 | KIECFDSVEISGVEDR | 586 | fnas | 146 |
| 16875.1 | −6.1 | 5.89 | 1754.79 | 0.00103 | 2 | 1 | yfkk | 572 | IECFDSVEISGVEDR | 586 | fnas | 147 |
| 14436.1 | −3.6 | 5.4 | 1352.633 | 0.0005 | 2 | 1 | kiec | 575 | FDSVEISGVEDR | 586 | fnas | 148 |
| 16668.1 | −4.9 | 5.69 | 1478.764 | 0.00334 | 2 | 0.667 | vedr | 587 | FNASLGTYHDLLK | 599 | iikd | 149 |
| 17620.1 | −4.8 | 5.74 | 1479.748 | 0.0015 | 2 | 0.667 | vedr | 587 | FNASLGTYHDLLK | 599 | iikd | 149 |
| 16665.1 | −4.3 | 6.06 | 1478.764 | 1.005 | 3 | 1 | vedr | 587 | FNASLGTYHDLLK | 599 | iikd | 149 |
| 16872.1 | −4.2 | 6.22 | 1478.764 | 0.00226 | 3 | 1 | vedr | 587 | FNASLGTYHDLLK | 599 | iikd | 149 |
| 16742.1 | −3.2 | 5.64 | 1217.652 | 0.00115 | 2 | 0.667 | drfn | 589 | ASLGTYHDLLK | 599 | iikd | 150 |
| 33981.1 | −7.7 | 5.83 | 3253.563 | 1.001 | 3 | 1 | kiik | 603 | DKDFLDNEENEDILE | 629 | emie | 151 |
| | | | | | | | | | DIVLTLTLFEDR | | | |
| 6293.1 | −2.2 | 6.01 | 838.3611 | −0.00083 | 2 | 1 | fedr | 630 | EMIEER | 635 | lkty | 152 |
| 12832.1 | −3.6 | 6.08 | 1350.705 | −0.00112 | 3 | 0.75 | ieer | 636 | LKTYAHLFDDK | 646 | vmkq | 153 |
| 11973.1 | −12.4 | 6.79 | 1483.725 | −0.0008 | 2 | 0.5 | erlk | 638 | TYAHLFDDKVMK | 649 | qlkr | 154 |
| 11928.1 | −3.9 | 6.37 | 1483.725 | 0.00022 | 3 | 0.75 | erlk | 638 | TYAHLFDDKVMK | 649 | qlkr | 154 |
| 18943.1 | −8.3 | 5.88 | 1724.831 | −0.00183 | 2 | 0.667 | fanr | 692 | NFMQLIHDDSLTFK | 705 | ediq | 155 |
| 18974.1 | −3.8 | 6.69 | 1724.831 | 0.00047 | 3 | 1 | fanr | 692 | NFMQLIHDDSLTFK | 705 | ediq | 155 |
| 19282.1 | −3.5 | 5.96 | 2338.138 | 1.002 | 3 | 0.75 | fanr | 692 | NFMQLIHDDSLTFKE | 710 | aqvs | 156 |
| | | | | | | | | | DIQK | | | |
| 8057.1 | −3.4 | 5.76 | 1227.56 | 0.00241 | 2 | 1 | diqk | 711 | AQVSGQGDSLHE | 722 | hian | 157 |
| 6773.1 | −4 | 5.49 | 1364.619 | 0.00002 | 2 | 0.667 | diqk | 711 | AQVSGQGDSLHEH | 723 | ianl | 158 |
| 6788.1 | −4 | 6.04 | 1364.619 | 0 | 3 | 1 | diqk | 711 | AQVSGQGDSLHEH | 723 | ianl | 158 |
| 9603.1 | −7.8 | 6.34 | 1548.74 | 0.00079 | 2 | 0.667 | diqk | 711 | AQVSGQGDSLHEHIA | 725 | nlag | 159 |
| 9602.1 | −3.5 | 6.54 | 1548.74 | 0.00145 | 3 | 1 | diqk | 711 | AQVSGQGDSLHEHIA | 725 | nlag | 159 |
| 9047.1 | −4.1 | 5.65 | 1662.783 | 0.00205 | 2 | 0.667 | diqk | 711 | AQVSGQGDSLHEHIA | 726 | lags | 160 |
| | | | | | | | | | N | | | |
| 14319.1 | −5.3 | 5.76 | 2400.227 | 0.00426 | 3 | 0.75 | diqk | 711 | AQVSGQGDSLHEHIA | 734 | kgil | 161 |
| | | | | | | | | | NLAGSPAIK | | | |
| 13049.1 | −4.6 | 5.87 | 2529.306 | 0.016 | 3 | 0.6 | diqk | 711 | AQVSGQGDSLHEHIA | 735 | gilq | 162 |
| | | | | | | | | | NLAGSPAIKK | | | |
| 9125.1 | −2.3 | 6.07 | 1452.763 | −0.00189 | 3 | 0.75 | vmgr | 754 | HKPENIVIEMAR | 765 | enqt | 163 |
| 17281.1 | −7.5 | 5.97 | 1556.889 | 0.00287 | 2 | 0.667 | rmkr | 784 | IEEGIKELGSQILK | 797 | ehpv | 164 |
| 17280.1 | −4 | 6.56 | 1556.889 | 0.00291 | 3 | 1 | rmkr | 784 | IEEGIKELGSQILK | 797 | ehpv | 164 |
| 7505.1 | −4.3 | 5.6 | 1565.755 | 0.00139 | 3 | 1 | qilk | 798 | EHPVENTQLQNEK | 810 | lyly | 165 |
| 18701.1 | −6.9 | 6.41 | 1303.668 | −0.00119 | 2 | 1 | ubek | 811 | LYLYYLQNGR | 820 | dmyv | 166 |
| 18272.1 | −4 | 6.34 | 1302.684 | 0.00236 | 2 | 1 | qnek | 811 | LYLYYLQNGR | 820 | dmyv | 166 |
| 14534.1 | −6.5 | 5.68 | 1526.679 | −0.00246 | 2 | 1 | qngr | 821 | DMYVDQELDINR | 832 | lsdy | 167 |
| 19802.1 | −7.8 | 5.66 | 1875.949 | 1.008 | 2 | 0.667 | dinr | 833 | LSDYDVDHIVPQSFLK | 848 | ddsi | 168 |
| 19790.1 | −5.4 | 6.23 | 1875.949 | 0.00269 | 3 | 1 | dinr | 833 | LSDYDVDHIVPQSFLK | 848 | ddsi | 168 |
| 9955.1 | −4 | 6.11 | 1202.59 | 0.00107 | 2 | 1 | nrgk | 867 | SDNVPSEEVVK | 877 | kmkn | 169 |
| 9613.1 | −5.4 | 6.56 | 1038.569 | 0.00069 | 2 | 0.667 | qitk | 930 | HVAQILDSR | 938 | mntk | 170 |
| 9444.1 | −5.9 | 5.9 | 1165.585 | 0.00011 | 2 | 0.667 | mntk | 943 | YDENDKLIR | 951 | evkv | 171 |
| 15410.1 | −2.8 | 6.1 | 1349.626 | 1.004 | 2 | 1 | kypk | 1004 | LESEFVYGDYK | 1014 | vydv | 172 |
| 7688.1 | −3.6 | 5.36 | 1181.616 | 0.00214 | 2 | 0.667 | nivk | 1097 | KTEVQTGGFSK | 1107 | esil | 173 |
| 13492.1 | −7.3 | 5.6 | 1300.649 | −0.00043 | 2 | 1 | nrgr | 1226 | GLSPDIADSVNGR | 1238 | ftis | 174 |
| 21034.1 | −4.3 | 5.68 | 2160.118 | 0.02 | 3 | 1 | tstk | 1476 | EVLDATLIHQSITGLY | 1494 | idls | 175 |
| | | | | | | | | | ETR | | | |

TABLE 11
Peptide fraction identified by the analysis of L1

| | | | | | | | | | | | | SEQ |
|---|---|---|---|---|---|---|---|---|---|---|---|---|
| | | | | | | | | | | | | ID |
| spectrum | log(e) | log(l) | m + h | delta | Z | zeta | pre | start | sequence | end | post | NO |
| 18612.1 | −2.2 | 5.94 | 1367.684 | −0.00124 | 2 | 1 | igtn | 15 | SVGWAVIT | 26 | vpsk | 306 |
| | | | | | | | | | DEYK | | | |
| 21294.1 | −13.9 | 6.13 | 2006.055 | 1.005 | 3 | 1 | hsik | 45 | KNLIGALL | 63 | lkrt | 307 |
| | | | | | | | | | FDSGETAE | | | |
| | | | | | | | | | ATR | | | |
| 21505.1 | −6.8 | 5.96 | 2006.055 | 1.002 | 3 | 1 | hsik | 45 | KNLIGALL | 63 | lkrt | 308 |
| | | | | | | | | | FDSGETAE | | | |
| | | | | | | | | | ATR | | | |
| 24454.1 | −14 | 8.18 | 1877.96 | −0.00276 | 2 | 1 | sikk | 46 | NLIGALLF | 63 | lkrt | 309 |
| | | | | | | | | | DSGETAEA | | | |
| | | | | | | | | | TR | | | |
| 24640.1 | −13.1 | 6.59 | 1877.96 | −0.00166 | 2 | 1 | sikk | 46 | NLIGALLF | 63 | lkrt | 310 |
| | | | | | | | | | DSGETAEA | | | |
| | | | | | | | | | TR | | | |
| 24839.1 | −8.5 | 6.02 | 1877.96 | −0.00154 | 2 | 1 | sikk | 46 | NLIGALLF | 63 | lkrt | 311 |
| | | | | | | | | | DSGETAEA | | | |
| | | | | | | | | | TR | | | |
| 24540.1 | −7.8 | 6.64 | 1877.96 | 1.001 | 3 | 1.5 | sikk | 46 | NLIGALLF | 63 | lkrt | 312 |
| | | | | | | | | | DSGETAEA | | | |
| | | | | | | | | | TR | | | |
| 25432.1 | −7.1 | 5.95 | 1877.96 | 0.988 | 2 | 1 | sikk | 46 | NLIGALLF | 63 | lkrt | 313 |
| | | | | | | | | | DSGETAEA | | | |
| | | | | | | | | | TR | | | |
| 24738.1 | −6.7 | 5.67 | 1877.96 | 0.00064 | 3 | 1.5 | sikk | 46 | NLIGALLF | 63 | lkrt | 314 |
| | | | | | | | | | DSGETAEA | | | |
| | | | | | | | | | TR | | | |
| 23171.1 | −5.7 | 5.84 | 1877.96 | −0.00265 | 3 | 1.5 | sikk | 46 | NLIGALLF | 63 | lkrt | 315 |
| | | | | | | | | | DSGETAEA | | | |
| | | | | | | | | | TR | | | |
| 24345.1 | −4 | 5.15 | 1877.96 | 0.00119 | 3 | 1.5 | sikk | 46 | NLIGALLF | 63 | lkrt | 316 |
| | | | | | | | | | DSGETAEA | | | |
| | | | | | | | | | TR | | | |
| 25226.1 | −3.9 | 5.51 | 1877.96 | 0.0009 | 2 | 1 | sikk | 46 | NLIGALLF | 63 | lkrt | 317 |
| | | | | | | | | | DSGETAEA | | | |
| | | | | | | | | | TR | | | |
| 25023.1 | −3.8 | 5.71 | 1877.96 | 1.002 | 2 | 1 | sikk | 46 | NLIGALLF | 63 | lkrt | 318 |
| | | | | | | | | | DSGETAEA | | | |
| | | | | | | | | | TR | | | |
| 24935.1 | −3.3 | 5.18 | 1877.96 | −0.00027 | 3 | 1.5 | sikk | 46 | NLIGALLF | 63 | lkrt | 319 |
| | | | | | | | | | DSGETAEA | | | |
| | | | | | | | | | TR | | | |
| 27006.1 | −3.2 | 5.48 | 1877.96 | 0.987 | 2 | 1 | sikk | 46 | NLIGALLF | 63 | lkrt | 320 |
| | | | | | | | | | DSGETAEA | | | |
| | | | | | | | | | TR | | | |
| 25629.1 | −2.5 | 5.34 | 1877.96 | 0.00273 | 2 | 1 | sikk | 46 | NLIGALLF | 63 | lkrt | 321 |
| | | | | | | | | | DSGETAEA | | | |
| | | | | | | | | | TR | | | |
| 23102.1 | −2.4 | 5.34 | 1877.96 | 0.0009 | 2 | 1 | sikk | 46 | NLIGALLF | 63 | lkrt | 322 |
| | | | | | | | | | DSGETAEA | | | |
| | | | | | | | | | TR | | | |
| 20895.1 | −11.3 | 6.89 | 1763.917 | 1.001 | 2 | 1 | ikkn | 47 | LIGALLFD | 63 | lkrt | 323 |
| | | | | | | | | | SGETAEAT | | | |
| | | | | | | | | | R | | | |
| 20899.1 | −8.5 | 6.91 | 1763.917 | −0.0017 | 2 | 1 | ikkn | 47 | LIGALLFD | 63 | lkrt | 324 |
| | | | | | | | | | SGETAEAT | | | |
| | | | | | | | | | R | | | |
| 21106.1 | −6.9 | 5.72 | 1763.917 | 1.007 | 2 | 1 | ikkn | 47 | LIGALLFD | 63 | lkrt | 325 |
| | | | | | | | | | SGETAEAT | | | |
| | | | | | | | | | R | | | |
| 17803.1 | −6.6 | 6.23 | 1650.833 | 0.998 | 2 | 1 | kknl | 48 | IGALLFDS | 63 | lkrt | 326 |
| | | | | | | | | | GETAEATR | | | |
| 18011.1 | −3.6 | 5.6 | 1650.833 | 1.003 | 2 | 1 | kknl | 48 | IGALLFDS | 63 | lkrt | 327 |
| | | | | | | | | | GETAEATR | | | |
| 15511.1 | −3.1 | 5.93 | 1480.728 | 0.998 | 2 | 1 | nlig | 50 | ALLFDSGE | 63 | lkrt | 328 |
| | | | | | | | | | TAEATR | | | |
| 14840.1 | −4.3 | 5.95 | 1409.69 | −0.00437 | 2 | 1 | liga | 51 | LLFDSGET | 63 | lkrt | 329 |
| | | | | | | | | | AEATR | | | |
| 7775.1 | −3.9 | 5.81 | 1183.523 | −0.00128 | 2 | 1 | gall | 53 | FDSGETAE | 63 | lkrt | 330 |
| | | | | | | | | | ATR | | | |
| 21402.1 | −9.2 | 6.53 | 1777.814 | −0.00013 | 2 | 1 | rknr | 79 | ICYLQEIF | 92 | vdds | 331 |
| | | | | | | | | | SNEMAK | | | |
| 20798.1 | −7.1 | 7.79 | 1761.819 | −0.00131 | 2 | 1 | rknr | 79 | ICYLQEIF | 92 | vdds | 332 |
| | | | | | | | | | SNEMAK | | | |
| 21129.1 | −3.7 | 5.57 | 1761.819 | 0.0026 | 2 | 1 | rknr | 79 | ICYLQEIF | 92 | vdds | 333 |
| | | | | | | | | | SNEMAK | | | |
| 11978.1 | −4.6 | 6.45 | 1022.469 | 0.00039 | 2 | 0.667 | emak | 93 | VDDSFFHR | 100 | lees | 334 |
| 12393.1 | −4.4 | 5.85 | 1022.469 | −0.0001 | 2 | 0.667 | emak | 93 | VDDSFFHR | 100 | lees | 335 |
| 12184.1 | −4.2 | 6.31 | 1022.469 | −0.00028 | 2 | 0.667 | emak | 93 | VDDSFFHR | 100 | lees | 336 |
| 16521.1 | −3.5 | 5.93 | 1135.553 | −0.00042 | 2 | 0.667 | emak | 93 | VDDSFFHR | 101 | eesf | 337 |
| | | | | | | | | | L | | | |
| 17555.1 | −4.2 | 5.56 | 1480.67 | −0.00106 | 2 | 0.667 | emak | 93 | VDDSFFHR | 104 | flve | 338 |
| | | | | | | | | | LEES | | | |
| 11987.1 | −2.6 | 6.24 | 923.4006 | −0.00065 | 2 | 0.667 | makv | 94 | DDSFFHR | 100 | lees | 339 |
| 15478.1 | −3.8 | 6.49 | 1337.647 | −0.00313 | 2 | 1 | ffhr | 101 | LEESFLVE | 111 | kher | 340 |
| | | | | | | | | | EDK | | | |
| 15271.1 | −3.5 | 6.56 | 1337.647 | −0.00093 | 2 | 1 | ffhr | 101 | LEESFLVE | 111 | kher | 341 |
| | | | | | | | | | EDK | | | |
| 13291.1 | −7.9 | 6.65 | 1465.742 | 0.00054 | 2 | 0.667 | ffhr | 101 | LEESFLVE | 112 | herh | 342 |
| | | | | | | | | | EDKK | | | |
| 13499.1 | −3.3 | 5.79 | 1465.742 | −0.0041 | 2 | 0.667 | ffhr | 101 | LEESFLVE | 112 | herh | 343 |
| | | | | | | | | | EDKK | | | |
| 13270.1 | −2.5 | 6.36 | 1465.742 | 0.00089 | 3 | 1 | ffhr | 101 | LEESFLVE | 112 | herh | 344 |
| | | | | | | | | | EDKK | | | |
| 10947.1 | −9.2 | 5.79 | 1887.945 | −0.00127 | 2 | 0.4 | ffhr | 101 | LEESFLVE | 115 | hpif | 345 |
| | | | | | | | | | EDKKHER | | | |
| 10948.1 | −8.7 | 6.94 | 1887.945 | 0.998 | 3 | 0.6 | ffhr | 101 | LEESFLVE | 115 | hpif | 346 |
| | | | | | | | | | EDKKHER | | | |
| 11148.1 | −5.1 | 5.87 | 1887.945 | 1.002 | 3 | 0.6 | ffhr | 101 | LEESFLVE | 115 | hpif | 347 |
| | | | | | | | | | EDKKHER | | | |
| 7069.1 | −3.1 | 5.67 | 1429.743 | 0.00029 | 3 | 0.6 | lees | 105 | FLVEEDKK | 115 | hpif | 348 |
| | | | | | | | | | HER | | | |
| 19397.1 | −3.5 | 5.96 | 1310.674 | 0.00105 | 2 | 1 | kher | 116 | HPIFGNIV | 127 | yhek | 349 |
| | | | | | | | | | DEVA | | | |
| 20629.1 | −3.3 | 5.76 | 1473.737 | −0.00075 | 2 | 1 | kher | 116 | HPIFGNIV | 128 | heky | 350 |
| | | | | | | | | | DEVAY | | | |
| 17825.1 | −7.8 | 6.63 | 1868.918 | 0.017 | 2 | 0.5 | kher | 116 | HPIFGNIV | 131 | ypti | 351 |
| | | | | | | | | | DEVAYHEK | | | |
| 18259.1 | −7.3 | 6.22 | 1867.934 | 1.001 | 3 | 0.75 | kher | 116 | HPIFGNIV | 131 | ypti | 352 |
| | | | | | | | | | DEVAYHEK | | | |
| 18905.1 | −4.7 | 6.46 | 1868.918 | 0.0024 | 3 | 0.75 | kher | 116 | HPIFGNIV | 131 | ypti | 353 |
| | | | | | | | | | DEVAYHEK | | | |
| 18469.1 | −4.7 | 5.97 | 1867.934 | −0.00033 | 3 | 0.75 | kher | 116 | HPIFGNIV | 131 | ypti | 354 |
| | | | | | | | | | DEVAYHEK | | | |
| 14886.1 | −4.5 | 6.01 | 1867.934 | −0.00253 | 3 | 0.75 | kher | 116 | HPIFGNIV | 131 | ypti | 355 |
| | | | | | | | | | DEVAYHEK | | | |
| 18048.1 | −4.2 | 6.62 | 1867.934 | 0.00021 | 3 | 0.75 | kher | 116 | HPIFGNIV | 131 | ypti | 356 |
| | | | | | | | | | DEVAYHEK | | | |
| 17839.1 | −4.2 | 7.97 | 1867.934 | 1.003 | 3 | 0.75 | kher | 116 | HPIFGNIV | 131 | ypti | 357 |
| | | | | | | | | | DEVAYHEK | | | |
| 17637.1 | −3.6 | 6.15 | 1867.934 | 0.00626 | 3 | 0.75 | kher | 116 | HPIFGNIV | 131 | ypti | 358 |
| | | | | | | | | | DEVAYHEK | | | |
| 19327.1 | −2.2 | 5.46 | 1867.934 | −0.00143 | 3 | 0.75 | kher | 116 | HPIFGNIV | 131 | ypti | 359 |
| | | | | | | | | | DEVAYHEK | | | |
| 19111.1 | −2 | 5.77 | 1868.918 | 0.00607 | 3 | 0.75 | kher | 116 | HPIFGNIV | 131 | ypti | 360 |
| | | | | | | | | | DEVAYHEK | | | |
| 19943.1 | −8 | 6.17 | 1730.875 | 0.00322 | 3 | 1 | herh | 117 | PIFGNIVD | 131 | ypti | 361 |
| | | | | | | | | | EVAYHEK | | | |
| 13025.1 | −5.2 | 7.32 | 1062.573 | −0.00053 | 2 | 0.667 | yhek | 132 | YPTIYHLR | 139 | kklv | 362 |
| 12827.1 | −4.7 | 6.8 | 1062.573 | −0.00089 | 2 | 0.667 | yhek | 132 | YPTIYHLR | 139 | kklv | 363 |
| 12618.1 | −4 | 6.12 | 1062.573 | 0.00008 | 2 | 0.667 | yhek | 132 | YPTIYHLR | 139 | kklv | 364 |
| 13260.1 | −3 | 5.88 | 1062.573 | −0.00175 | 2 | 0.667 | yhek | 132 | YPTIYHLR | 139 | kklv | 365 |
| 7865.1 | −7.4 | 6.11 | 1360.743 | 0.00026 | 3 | 0.75 | hlrk | 141 | KLVDSTDK | 152 | liyl | 366 |
| | | | | | | | | | ADLR | | | |
| 7866.1 | −4.5 | 5.5 | 1360.743 | −0.00022 | 2 | 0.5 | hlrk | 141 | KLVDSTDK | 152 | liyl | 367 |
| | | | | | | | | | ADLR | | | |
| 8274.1 | −5 | 5.73 | 1232.648 | 0.00063 | 2 | 0.667 | lrkk | 142 | LVDSTDKA | 152 | liyl | 368 |
| | | | | | | | | | DLR | | | |
| 8092.1 | −4.3 | 6.23 | 1232.648 | −0.00262 | 3 | 1 | lrkk | 142 | LVDSTDKA | 152 | liyl | 369 |
| | | | | | | | | | DLR | | | |
| 8327.1 | −2.2 | 5.49 | 1232.648 | −0.00244 | 3 | 1 | lrkk | 142 | LVDSTDKA | 152 | liyl | 370 |
| | | | | | | | | | DLR | | | |
| 18084.1 | −3.1 | 5.64 | 913.5506 | −0.001 | 1 | 0.5 | adlr | 153 | LIYLALAH | 160 | mikf | 371 |
| 17808.1 | −7.6 | 7.4 | 1301.765 | 0.00008 | 2 | 0.667 | adlr | 153 | LIYLALAH | 163 | frgh | 372 |
| | | | | | | | | | MIK | | | |
| 17599.1 | −7.4 | 6.29 | 1301.765 | 0.00032 | 2 | 0.667 | adlr | 153 | LIYLALAH | 163 | frgh | 373 |
| | | | | | | | | | MIK | | | |
| 18840.1 | −7.4 | 6.6 | 1317.76 | −0.00009 | 2 | 0.667 | adlr | 153 | LIYLALAH | 163 | frgh | 374 |
| | | | | | | | | | MIK | | | |
| 18776.1 | −3.2 | 5.89 | 1317.76 | 0.00042 | 3 | 1 | adlr | 153 | LIYLALAH | 163 | frgh | 375 |
| | | | | | | | | | MIK | | | |
| 20855.1 | −2.5 | 6.87 | 1285.77 | −0.00029 | 3 | 1 | adlr | 153 | LIYLALAH | 163 | frgh | 376 |
| | | | | | | | | | MIK | | | |
| 17597.1 | −2.3 | 6.84 | 1301.765 | 0.00034 | 3 | 1 | adlr | 153 | LIYLALAH | 163 | frgh | 377 |
| | | | | | | | | | MIK | | | |
| 19208.1 | −2.2 | 5.9 | 1317.76 | 0.00042 | 3 | 1 | adlr | 153 | LIYLALAH | 163 | frgh | 378 |
| | | | | | | | | | MIK | | | |
| 21051.1 | −2.1 | 5.68 | 1285.77 | 0.00036 | 2 | 0.667 | adlr | 153 | LIYLALAH | 163 | frgh | 379 |
| | | | | | | | | | MIK | | | |
| 17807.1 | −2.1 | 7.83 | 1301.765 | 0.00052 | 3 | 1 | adlr | 153 | LIYLALAH | 163 | frgh | 380 |
| | | | | | | | | | MIK | | | |
| 17631.1 | −2 | 6.06 | 1317.76 | −0.00045 | 2 | 0.667 | adlr | 153 | LIYLALAH | 163 | frgh | 381 |
| | | | | | | | | | MIK | | | |
| 15745.1 | −15 | 6.14 | 1984.925 | 1.005 | 2 | 0.667 | ikfr | 166 | GHFLIEGD | 183 | lfiq | 382 |
| | | | | | | | | | LNPDNSDV | | | |
| | | | | | | | | | DK | | | |
| 15724.1 | −9.1 | 6.34 | 1984.925 | 1.001 | 3 | 1 | ikfr | 166 | GHFLIEGD | 183 | lfiq | 383 |
| | | | | | | | | | LNPDNSDV | | | |
| | | | | | | | | | DK | | | |
| 15951.1 | −3.1 | 5.45 | 1984.925 | 0.00543 | 2 | 0.667 | ikfr | 166 | GHFLIEGD | 183 | lfiq | 384 |
| | | | | | | | | | LNPDNSDV | | | |
| | | | | | | | | | DK | | | |
| 16211.1 | −2.2 | 5.55 | 1984.925 | 1.006 | 3 | 1 | ikfr | 166 | GHFLIEGD | 183 | lfiq | 385 |
| | | | | | | | | | LNPDNSDV | | | |
| | | | | | | | | | DK | | | |
| 16358.1 | −4.1 | 5.59 | 1845.898 | −0.0029 | 2 | 1 | vqty | 193 | NQLFEENP | 209 | ails | 386 |
| | | | | | | | | | INASGVDA | | | |
| | | | | | | | | | K | | | |
| 16105.1 | −10.7 | 6.27 | 1731.855 | −0.00087 | 2 | 1 | qtyn | 194 | QLFEENPI | 209 | ails | 387 |
| | | | | | | | | | NASGVDAK | | | |
| 18787.1 | −3.2 | 5.94 | 1223.711 | 0.00132 | 2 | 1 | sksr | 221 | RLENLIAQ | 231 | ekkn | 388 |
| | | | | | | | | | LPG | | | |
| 17397.1 | −12 | 6.71 | 1481.832 | −0.00075 | 2 | 0.667 | sksr | 221 | RLENLIAQ | 233 | kngl | 389 |
| | | | | | | | | | LPGEK | | | |
| 16544.1 | −8.3 | 6.89 | 1480.848 | −0.00037 | 2 | 0.667 | sksr | 221 | RLENLIAQ | 233 | kngl | 390 |
| | | | | | | | | | LPGEK | | | |
| 16968.1 | −7.6 | 6.58 | 1480.848 | 0.00059 | 3 | 1 | sksr | 221 | RLENLIAQ | 233 | kngl | 391 |
| | | | | | | | | | LPGEK | | | |
| 16554.1 | −7.6 | 7.86 | 1480.848 | −0.00124 | 3 | 1 | sksr | 221 | RLENLIAQ | 233 | kngl | 392 |
| | | | | | | | | | LPGEK | | | |
| 17355.1 | −6.5 | 6.93 | 1481.832 | −0.00024 | 3 | 1 | sksr | 221 | RLENLIAQ | 233 | kngl | 393 |
| | | | | | | | | | LPGEK | | | |
| 16957.1 | −4.8 | 6.04 | 1480.848 | 0.00012 | 2 | 0.667 | sksr | 221 | RLENLIAQ | 233 | kngl | 394 |
| | | | | | | | | | LPGEK | | | |
| 16758.1 | −4.3 | 7.51 | 1480.848 | −0.00096 | 3 | 1 | sksr | 221 | RLENLIAQ | 233 | kngl | 395 |
| | | | | | | | | | LPGEK | | | |
| 16747.1 | −4.2 | 7.14 | 1480.848 | −0.00147 | 2 | 0.667 | sksr | 221 | RLENLIAQ | 233 | kngl | 396 |
| | | | | | | | | | LPGEK | | | |
| 18174.1 | −2.9 | 6.11 | 1481.832 | −0.00125 | 3 | 1 | sksr | 221 | RLENLIAQ | 233 | kngl | 397 |
| | | | | | | | | | LPGEK | | | |
| 17613.1 | −2.9 | 5.94 | 1481.832 | 0.013 | 3 | 1 | sksr | 221 | RLENLIAQ | 233 | kngl | 398 |
| | | | | | | | | | LPGEK | | | |
| 15095.1 | −7.6 | 7.07 | 1608.943 | 1.002 | 3 | 0.75 | sksr | 221 | RLENLIAQ | 234 | nglf | 399 |
| | | | | | | | | | LPGEKK | | | |
| 15821.1 | −7.4 | 6.65 | 1609.927 | −0.00093 | 3 | 0.75 | sksr | 221 | RLENLIAQ | 234 | nglf | 400 |
| | | | | | | | | | LPGEKK | | | |
| 15108.1 | −6.4 | 6.16 | 1608.943 | −0.00146 | 2 | 0.5 | sksr | 221 | RLENLIAQ | 234 | nglf | 401 |
| | | | | | | | | | LPGEKK | | | |
| 17883.1 | −8.5 | 6.86 | 1324.747 | −0.00034 | 2 | 1 | ksrr | 222 | LENLIAQL | 233 | kngl | 402 |
| | | | | | | | | | PGEK | | | |
| 18479.1 | −5.2 | 6.23 | 1325.731 | −0.00364 | 2 | 1 | ksrr | 222 | LENLIAQL | 233 | kngl | 403 |
| | | | | | | | | | PGEK | | | |
| 17676.1 | −4.1 | 6.7 | 1324.747 | 0.00027 | 2 | 1 | ksrr | 222 | LENLIAQL | 233 | kngl | 404 |
| | | | | | | | | | PGEK | | | |
| 17732.1 | −3.7 | 5.92 | 1324.747 | 0.00023 | 1 | 0.5 | ksrr | 222 | LENLIAQL | 233 | kngl | 405 |
| | | | | | | | | | PGEK | | | |
| 18139.1 | −3.5 | 6.3 | 1325.73 | 0.00173 | 2 | 1 | ksrr | 222 | LENLIAQL | 233 | kngl | 406 |
| | | | | | | | | | PGEK | | | |
| 17735.1 | −2.3 | 6.14 | 1324.747 | −0.00139 | 3 | 1 | ksrr | 222 | LENLIAQL | 233 | kngl | 407 |
| | | | | | | | | | PGEK | | | |
| 15931.1 | −8.8 | 6.28 | 1452.842 | −0.00057 | 2 | 0.667 | ksrr | 222 | LENLIAQL | 234 | nglf | 408 |
| | | | | | | | | | PGEKK | | | |
| 15904.1 | −2.8 | 6.03 | 1452.842 | 0.00039 | 3 | 1 | ksrr | 222 | LENLIAQL | 234 | nglf | 409 |
| | | | | | | | | | PGEKK | | | |
| 12863.1 | −2.9 | 6.16 | 968.5775 | −0.00073 | 2 | 1 | rlen | 225 | LIAQLPGE | 233 | kngl | 410 |
| | | | | | | | | | K | | | |
| 22243.1 | −2.4 | 5.75 | 1386.836 | 0.00035 | 2 | 1 | lfgn | 241 | LIALSLGL | 253 | snfd | 411 |
| | | | | | | | | | TPNFK | | | |
| 17859.1 | −2.3 | 5.89 | 1089.63 | 0.00369 | 2 | 1 | nlia | 244 | LSLGLTPN | 253 | snfd | 412 |
| | | | | | | | | | FK | | | |
| 11997.1 | −5 | 6.02 | 1109.511 | 0.0009 | 1 | 0.5 | pnfk | 254 | SNFDLAED | 263 | lqls | 413 |
| | | | | | | | | | AK | | | |
| 12794.1 | −3.3 | 6.24 | 1110.495 | 0.0007 | 2 | 1 | pnfk | 254 | SNFDLAED | 263 | lqls | 414 |
| | | | | | | | | | AK | | | |
| 12084.1 | −3 | 8.32 | 1109.511 | 0.00071 | 2 | 1 | pnfk | 254 | SNFDLAED | 263 | lqls | 415 |
| | | | | | | | | | AK | | | |
| 12294.1 | −2.5 | 6.43 | 1109.511 | −0.00186 | 2 | 1 | pnfk | 254 | SNFDLAED | 263 | lqls | 416 |
| | | | | | | | | | AK | | | |
| 11267.1 | −2.4 | 5.79 | 1109.511 | −0.00052 | 2 | 1 | pnfk | 254 | SNFDLAED | 263 | lqls | 417 |
| | | | | | | | | | AK | | | |
| 13194.1 | −2.3 | 5.86 | 1110.495 | −0.00138 | 2 | 1 | pnfk | 254 | SNFDLAED | 263 | lqls | 418 |
| | | | | | | | | | AK | | | |
| 12503.1 | −2 | 5.86 | 1109.511 | −0.00381 | 2 | 1 | pnfk | 254 | SNFDLAED | 263 | lqls | 419 |
| | | | | | | | | | AK | | | |
| 15575.1 | −3.2 | 5.86 | 1350.654 | 0.00479 | 2 | 1 | pnfk | 254 | SNFDLAED | 265 | lskd | 420 |
| | | | | | | | | | AKLQ | | | |
| 16830.1 | −7.7 | 5.89 | 1679.849 | 0.022 | 3 | 1 | pnfk | 254 | SNFDLAED | 268 | dtyd | 421 |
| | | | | | | | | | AKLQLSK | | | |
| 20189.1 | −3.3 | 5.47 | 1424.742 | −0.00196 | 2 | 1 | llaq | 282 | IGDQYADL | 294 | nlsd | 422 |
| | | | | | | | | | FLAAK | | | |
| 18997.1 | −2.4 | 5.6 | 1254.636 | 0.00273 | 2 | 1 | aqig | 284 | DQYADLFL | 294 | nlsd | 423 |
| | | | | | | | | | AAK | | | |
| 24981.1 | −9.2 | 7.46 | 1442.821 | −0.00022 | 2 | 1 | laak | 295 | NLSDAILL | 307 | vnte | 424 |
| | | | | | | | | | SDILR | | | |
| 24795.1 | −8.4 | 8 | 1442.821 | 0.00063 | 2 | 1 | laak | 295 | NLSDAILL | 307 | vnte | 425 |
| | | | | | | | | | SDILR | | | |
| 25186.1 | −7.9 | 6.52 | 1442.821 | −0.00022 | 2 | 1 | laak | 295 | NLSDAILL | 307 | vnte | 426 |
| | | | | | | | | | SDILR | | | |
| 26380.1 | −7.5 | 5.94 | 1443.805 | 0.00221 | 2 | 1 | laak | 295 | NLSDAILL | 307 | vnte | 427 |
| | | | | | | | | | SDILR | | | |
| 25389.1 | −5.7 | 6.1 | 1442.821 | −0.00022 | 2 | 1 | laak | 295 | NLSDAILL | 307 | vnte | 428 |
| | | | | | | | | | SDILR | | | |
| 24598.1 | −5.3 | 5.73 | 1442.821 | −0.0023 | 2 | 1 | laak | 295 | NLSDAILL | 307 | vnte | 429 |
| | | | | | | | | | SDILR | | | |
| 25585.1 | −3.9 | 5.85 | 1442.821 | 0.00149 | 2 | 1 | laak | 295 | NLSDAILL | 307 | vnte | 430 |
| | | | | | | | | | SDILR | | | |
| 25788.1 | −2.7 | 5.53 | 1442.821 | 0.00075 | 2 | 1 | laak | 295 | NLSDAILL | 307 | vnte | 431 |
| | | | | | | | | | SDILR | | | |
| 27504.1 | −2.7 | 5.73 | 1443.805 | 1.003 | 2 | 1 | laak | 295 | NLSDAILL | 307 | vnte | 432 |
| | | | | | | | | | SDILR | | | |
| 24784.1 | −2.6 | 6.5 | 1442.821 | −0.00115 | 3 | 1.5 | laak | 295 | NLSDAILL | 307 | vnte | 433 |
| | | | | | | | | | SDILR | | | |
| 25985.1 | −2.5 | 5.54 | 1442.821 | 0.00027 | 2 | 1 | laak | 295 | NLSDAILL | 307 | vnte | 434 |
| | | | | | | | | | SDILR | | | |
| 26184.1 | −2.4 | 5.55 | 1442.821 | 1.003 | 2 | 1 | laak | 295 | NLSDAILL | 307 | vnte | 435 |
| | | | | | | | | | SDILR | | | |
| 23974.1 | −2.9 | 5.37 | 1328.778 | 0.00132 | 2 | 1 | aakn | 296 | LSDAILLS | 307 | vnte | 436 |
| | | | | | | | | | DILR | | | |
| 22265.1 | −4.7 | 6.13 | 1215.694 | −0.00006 | 2 | 1 | aknl | 297 | SDAILLSD | 307 | vnte | 437 |
| | | | | | | | | | ILR | | | |
| 21354.1 | −2 | 5.59 | 1013.635 | −0.00023 | 2 | 1 | nlsd | 299 | AILLSDIL | 307 | vnte | 438 |
| | | | | | | | | | R | | | |
| 10378.1 | −4.6 | 5.91 | 949.5023 | −0.00145 | 2 | 1 | eitk | 315 | APLSASMI | 323 | ryde | 439 |
| | | | | | | | | | K | | | |
| 12361.1 | −4.3 | 5.62 | 917.5125 | −0.00068 | 1 | 0.5 | eitk | 315 | APLSASMI | 323 | ryde | 440 |
| | | | | | | | | | K | | | |
| 12329.1 | −3.9 | 6.48 | 917.5125 | 0.00047 | 2 | 1 | eitk | 315 | APLSASMI | 323 | ryde | 441 |
| | | | | | | | | | K | | | |
| 9556.1 | −3 | 5.64 | 933.5074 | −0.00006 | 2 | 1 | eitk | 315 | APLSASMI | 323 | ryde | 222 |
| | | | | | | | | | K | | | |
| 9764.1 | −2.4 | 5.95 | 933.5074 | −0.00061 | 2 | 1 | eitk | 315 | APLSASMI | 323 | ryde | 222 |
| | | | | | | | | | K | | | |
| 8469.1 | −5.8 | 6.11 | 1089.608 | 0.00082 | 2 | 0.667 | eitk | 315 | APLSASMI | 324 | ydeh | 223 |
| | | | | | | | | | KR | | | |
| 10647.1 | −8.7 | 6.87 | 1667.85 | −0.00053 | 3 | 0.6 | smik | 324 | RYDEHHQD | 336 | alvr | 224 |
| | | | | | | | | | LTLLK | | | |
| 10703.1 | −5.4 | 6.34 | 1668.834 | 0.017 | 2 | 0.4 | smik | 324 | RYDEHHQD | 336 | alvr | 224 |
| | | | | | | | | | LTLLK | | | |
| 11693.1 | −8.6 | 6.07 | 1511.749 | 0.00093 | 2 | 0.5 | mikr | 325 | YDEHHQDL | 336 | alvr | 225 |
| | | | | | | | | | TLLK | | | |
| 11692.1 | −5.5 | 6.72 | 1511.749 | 0.0016 | 3 | 0.75 | mikr | 325 | YDEHHQDL | 336 | alvr | 225 |
| | | | | | | | | | TLLK | | | |
| 11895.1 | −3.7 | 6.03 | 1511.749 | 0.00134 | 3 | 0.75 | mikr | 325 | YDEHHQDL | 336 | alvr | 225 |
| | | | | | | | | | TLLK | | | |
| 12308.1 | −2.6 | 6 | 1512.733 | 0.00023 | 3 | 0.75 | mikr | 325 | YDEHHQDL | 336 | alvr | 255 |
| | | | | | | | | | TLLK | | | |
| 11321.1 | −2.8 | 5.65 | 967.5571 | 0.00093 | 2 | 0.67 | ydeh | 329 | HQDLTLLK | 336 | alvr | 226 |
| 7208.1 | −2.7 | 6.03 | 1033.568 | −0.00085 | 2 | 0.67 | alvr | 341 | QQLPEKYK | 348 | eiff | 227 |
| 15756.1 | −2 | 6.19 | 2028.044 | 1.003 | 3 | 0.75 | alvr | 341 | QQLPEKYK | 356 | ngya | 228 |
| | | | | | | | | | EIFFDQSK | | | |
| 13718.1 | −3.5 | 6.13 | 1304.652 | 0.00027 | 2 | 0.67 | lpek | 347 | YKEIFFDQ | 356 | ngya | 229 |
| | | | | | | | | | SK | | | |
| 13511.1 | −3.2 | 6.92 | 1304.652 | 0.00015 | 2 | 0.67 | lpek | 347 | YKEIFFDQ | 356 | ngya | 229 |
| | | | | | | | | | SK | | | |
| 13927.1 | −2.9 | 5.59 | 1304.652 | 0.00259 | 2 | 0.67 | lpek | 347 | YKEIFFDQ | 356 | ngya | 229 |
| | | | | | | | | | SK | | | |
| 13918.1 | −3 | 6.36 | 1013.494 | 0.00078 | 2 | 1 | ekyk | 349 | EIFFDQSK | 356 | ngya | 230 |
| 14125.1 | −2.4 | 6.75 | 1013.494 | −0.00013 | 2 | 1 | ekyk | 349 | EIFFDQSK | 356 | ngya | 230 |
| 14334.1 | −2.2 | 6.27 | 1013.494 | 0.011 | 2 | 1 | ekyk | 349 | EIFFDQSK | 356 | ngya | 230 |
| 16869.1 | −14.9 | 5.97 | 1969.845 | 0.00378 | 2 | 1 | dqsk | 357 | NGYAGYID | 374 | fikp | 231 |
| | | | | | | | | | GGASQEEF | | | |
| | | | | | | | | | YK | | | |
| 16665.1 | −14 | 6.52 | 1969.845 | −0.00257 | 2 | 1 | dqsk | 357 | NGYAGYID | 374 | fikp | 231 |
| | | | | | | | | | GGASQEEF | | | |
| | | | | | | | | | YK | | | |
| 17529.1 | −11.4 | 5.98 | 1969.845 | 0.00024 | 2 | 1 | dqsk | 357 | NGYAGYID | 374 | fikp | 231 |
| | | | | | | | | | GGASQEEF | | | |
| | | | | | | | | | YK | | | |
| 16260.1 | −9.9 | 6.32 | 1968.86 | −0.00085 | 2 | 1 | dqsk | 357 | NGYAGYID | 374 | fikp | 231 |
| | | | | | | | | | GGASQEEF | | | |
| | | | | | | | | | YK | | | |
| 17323.1 | −9.9 | 5.98 | 1969.845 | 0.998 | 2 | 1 | dqsk | 357 | NGYAGYID | 374 | fikp | 231 |
| | | | | | | | | | GGASQEEF | | | |
| | | | | | | | | | YK | | | |
| 17410.1 | −6.8 | 6.49 | 1969.845 | 1.002 | 3 | 1.5 | dqsk | 357 | NGYAGYID | 374 | fikp | 231 |
| | | | | | | | | | GGASQEEF | | | |
| | | | | | | | | | YK | | | |
| 15624.1 | −6.2 | 5.88 | 1968.861 | 1.001 | 2 | 1 | dqsk | 357 | NGYAGYID | 374 | fikp | 231 |
| | | | | | | | | | GGASQEEF | | | |
| | | | | | | | | | YK | | | |
| 16814.1 | −6.2 | 5.78 | 1969.845 | −0.00374 | 3 | 1.5 | dqsk | 357 | NGYAGYID | 374 | fikp | 231 |
| | | | | | | | | | GGASQEEF | | | |
| | | | | | | | | | YK | | | |
| 16107.1 | −4.6 | 6.64 | 1968.861 | −0.00117 | 3 | 1.5 | dqsk | 357 | NGYAGYID | 374 | fikp | 231 |
| | | | | | | | | | GGASQEEF | | | |
| | | | | | | | | | YK | | | |
| 15831.1 | −4 | 5.53 | 1968.861 | 0.999 | 2 | 1 | dqsk | 357 | NGYAGYID | 374 | fikp | 231 |
| | | | | | | | | | GGASQEEF | | | |
| | | | | | | | | | YK | | | |
| 16699.1 | −3.8 | 5.78 | 1969.845 | 1 | 3 | 1.5 | dqsk | 357 | NGYAGYID | 374 | fikp | 231 |
| | | | | | | | | | GGASQEEF | | | |
| | | | | | | | | | YK | | | |
| 16465.1 | −3.1 | 5.74 | 1969.845 | 0.018 | 2 | 1 | dqsk | 357 | NGYAGYID | 374 | fikp | 231 |
| | | | | | | | | | GGASQEEF | | | |
| | | | | | | | | | YK | | | |
| 16055.1 | −2.3 | 5.35 | 1968.861 | 1.005 | 2 | 1 | dqsk | 357 | NGYAGYID | 374 | fikp | 231 |
| | | | | | | | | | GGASQEEF | | | |
| | | | | | | | | | YK | | | |
| 15836.1 | −2.5 | 5.5 | 1854.818 | −0.00565 | 2 | 1 | qskn | 358 | GYAGYIDG | 374 | fikp | 232 |
| | | | | | | | | | GASQEEFY | | | |
| | | | | | | | | | K | | | |
| 15101.1 | −3.1 | 5.74 | 1797.797 | 1.003 | 2 | 1 | skng | 359 | YAGYIDGG | 374 | fikp | 233 |
| | | | | | | | | | ASQEEFYK | | | |
| 11266.1 | −2.3 | 5.64 | 1343.611 | −0.00733 | 2 | 1 | yagy | 363 | IDGGASQE | 374 | fikp | 234 |
| | | | | | | | | | EFYK | | | |
| 11974.1 | −6.2 | 7.12 | 987.6237 | 0.00029 | 2 | 0.667 | efyk | 375 | FIKPILEK | 382 | mdgt | 235 |
| 11983.1 | −4.4 | 6.07 | 987.6237 | −0.00062 | 1 | 0.333 | efyk | 375 | FIKPILEK | 382 | mdgt | 235 |
| 12185.1 | −3.7 | 6.45 | 987.6237 | −0.00057 | 2 | 0.667 | efyk | 375 | FIKPILEK | 382 | mdgt | 235 |
| 12396.1 | −2.3 | 5.77 | 987.6237 | −0.00032 | 2 | 0.667 | efyk | 375 | FIKPILEK | 382 | mdgt | 235 |
| 16150.1 | −6.6 | 6.1 | 2119.172 | −0.00144 | 2 | 0.5 | efyk | 375 | FIKPILEK | 392 | lnre | 236 |
| | | | | | | | | | MDGTEELL | | | |
| | | | | | | | | | VK | | | |
| 16108.1 | −3.6 | 6.32 | 2119.172 | 1.002 | 3 | 0.75 | efyk | 375 | FIKPILEK | 392 | lnre | 236 |
| | | | | | | | | | MDGTEELL | | | |
| | | | | | | | | | VK | | | |
| 13017.1 | −5 | 6.93 | 1166.561 | 0.00016 | 2 | 1 | ilek | 383 | MDGTEELL | 392 | lnre | 237 |
| | | | | | | | | | VK | | | |
| 12648.1 | −4.8 | 6.8 | 1150.566 | −0.00078 | 2 | 1 | ilek | 383 | MDGTEELL | 392 | lnre | 237 |
| | | | | | | | | | VK | | | |
| 12437.1 | −4.7 | 7.54 | 1150.566 | −0.00029 | 2 | 1 | ilek | 383 | MDGTEELL | 392 | lnre | 237 |
| | | | | | | | | | VK | | | |
| 13554.1 | −4 | 5.41 | 1150.566 | 0.00142 | 2 | 1 | ilek | 383 | MDGTEELL | 392 | lnre | 237 |
| | | | | | | | | | VK | | | |
| 13345.1 | −2.8 | 5.91 | 1150.566 | −0.00029 | 2 | 1 | ilek | 383 | MDGTEELL | 392 | lnre | 237 |
| | | | | | | | | | VK | | | |
| 13940.1 | −2.3 | 6.3 | 1134.571 | 0.00097 | 2 | 1 | ilek | 383 | MDGTEELL | 392 | lnre | 237 |
| | | | | | | | | | VK | | | |
| 18750.1 | −12.9 | 6.56 | 2369.236 | 1.002 | 3 | 0.6 | rkqr | 404 | TFDNGSIP | 424 | rqed | 238 |
| | | | | | | | | | HQIHLGEL | | | |
| | | | | | | | | | HAILR | | | |
| 18061.1 | −10.4 | 6.25 | 2368.252 | 0.999 | 3 | 0.6 | rkqr | 404 | TFDNGSIP | 424 | rqed | 238 |
| | | | | | | | | | HQIHLGEL | | | |
| | | | | | | | | | HAILR | | | |
| 18547.1 | −6.6 | 5.86 | 2370.22 | 0.023 | 3 | 0.6 | rkqr | 404 | TFDNGSIP | 424 | rqed | 238 |
| | | | | | | | | | HQIHLGEL | | | |
| | | | | | | | | | HAILR | | | |
| 18095.1 | −5.7 | 5.87 | 2368.252 | 1.004 | 2 | 0.4 | rkqr | 404 | TFDNGSIP | 424 | rqed | 238 |
| | | | | | | | | | HQIHLGEL | | | |
| | | | | | | | | | HAILR | | | |
| 18618.1 | −2.2 | 5.49 | 2370.22 | 1.02 | 2 | 0.4 | rkqr | 404 | TFDNGSIP | 424 | rqed | 238 |
| | | | | | | | | | HQIHLGEL | | | |
| | | | | | | | | | HAILR | | | |
| 15980.1 | −3.3 | 6.06 | 1399.817 | 1.004 | 3 | 0.75 | siph | 413 | QIHLGELH | 424 | rqed | 239 |
| | | | | | | | | | AILR | | | |
| 17831.1 | −2.7 | 6.3 | 1101.5 | −0.0003 | 2 | 1 | ailr | 425 | RQEDFYPF | 432 | lkdn | 240 |
| 16705.1 | −5.8 | 8.06 | 1342.679 | −0.00184 | 2 | 0.667 | ailr | 425 | RQEDFYPF | 434 | dnre | 241 |
| | | | | | | | | | LK | | | |
| 17268.1 | −4.9 | 6.96 | 1343.663 | −0.00161 | 3 | 1 | ailr | 425 | RQEDFYPF | 434 | dnre | 241 |
| | | | | | | | | | LK | | | |
| 16913.1 | −4.8 | 7.08 | 1342.679 | 0.00012 | 2 | 0.667 | ailr | 425 | RQEDFYPF | 434 | dnre | 241 |
| | | | | | | | | | LK | | | |
| 16847.1 | −4.4 | 7.94 | 1342.679 | 0.00096 | 3 | 1 | ailr | 425 | RQEDFYPF | 434 | dnre | 241 |
| | | | | | | | | | LK | | | |
| 17122.1 | −4.1 | 6.24 | 1342.679 | −0.00037 | 2 | 0.667 | ailr | 425 | RQEDFYPF | 434 | dnre | 241 |
| | | | | | | | | | LK | | | |
| 17056.1 | −3.9 | 6.72 | 1342.679 | −0.00041 | 3 | 1 | ailr | 425 | RQEDFYPF | 434 | dnre | 241 |
| | | | | | | | | | LK | | | |
| 17331.1 | −3.9 | 6.34 | 1343.663 | −0.00221 | 2 | 0.667 | ailr | 425 | RQEDFYPF | 434 | dnre | 241 |
| | | | | | | | | | LK | | | |
| 16643.1 | −3.3 | 7.34 | 1342.679 | 1.002 | 3 | 1 | ailr | 425 | RQEDFYPF | 434 | dnre | 241 |
| | | | | | | | | | LK | | | |
| 17527.1 | −2.2 | 6.09 | 1342.679 | 0.0005 | 3 | 1 | ailr | 425 | RQEDFYPF | 434 | dnre | 241 |
| | | | | | | | | | LK | | | |
| 14748.1 | −6.5 | 6.42 | 1727.85 | 0.0002 | 3 | 0.75 | ailr | 425 | RQEDFYPF | 437 | ekie | 242 |
| | | | | | | | | | LKDNR | | | |
| 14778.1 | −2.7 | 5.74 | 1727.85 | 0.00113 | 2 | 0.5 | ailr | 425 | RQEDFYPF | 437 | ekie | 242 |
| | | | | | | | | | LKDNR | | | |
| 14962.1 | −2.4 | 6.13 | 1727.85 | 1.004 | 3 | 0.75 | ailr | 425 | RQEDFYPF | 437 | ekie | 242 |
| | | | | | | | | | LKDNR | | | |
| 15240.1 | −2.3 | 5.98 | 1728.834 | 1.001 | 3 | 0.75 | ailr | 425 | RQEDFYPF | 437 | ekie | 242 |
| | | | | | | | | | LKDNR | | | |
| 19016.1 | −3.4 | 7.45 | 1186.578 | −0.00143 | 2 | 1 | ilrr | 426 | QEDFYPFL | 434 | dnre | 243 |
| | | | | | | | | | K | | | |
| 19220.1 | −2.6 | 6.38 | 1186.578 | −0.00033 | 2 | 1 | ilrr | 426 | QEDFYPFL | 434 | dnre | 243 |
| | | | | | | | | | K | | | |
| 16515.1 | −5.5 | 6.03 | 1571.749 | −0.00261 | 2 | 0.667 | ilrr | 426 | QEDFYPFL | 437 | ekie | 244 |
| | | | | | | | | | KDNR | | | |
| 16482.1 | −4.2 | 6.14 | 1572.733 | 0.021 | 3 | 1 | ilrr | 426 | QEDFYPFL | 437 | ekie | 244 |
| | | | | | | | | | KDNR | | | |
| 22320.1 | −3.6 | 5.88 | 1779.032 | −0.00176 | 3 | 1 | kiek | 443 | ILTFRIPY | 457 | gnsr | 245 |
| | | | | | | | | | YVGPLAR | | | |
| 16946.1 | −5.2 | 6.95 | 1148.646 | 0.00004 | 2 | 1 | ltfr | 448 | IPYYVGPL | 457 | gnsr | 246 |
| | | | | | | | | | AR | | | |
| 16736.1 | −4.7 | 8.1 | 1148.646 | −0.00021 | 2 | 1 | ltfr | 448 | IPYYVGPL | 457 | gnsr | 246 |
| | | | | | | | | | AR | | | |
| 16533.1 | −4 | 6.19 | 1148.646 | −0.00265 | 2 | 1 | ltfr | 448 | IPYYVGPL | 457 | gnsr | 246 |
| | | | | | | | | | AR | | | |
| 17953.1 | −3.9 | 5.8 | 1148.646 | 0.00016 | 2 | 1 | ltfr | 448 | IPYYVGPL | 457 | gnsr | 246 |
| | | | | | | | | | AR | | | |
| 17518.1 | −3.8 | 6.01 | 1148.646 | 0.00028 | 2 | 1 | ltfr | 448 | IPYYVGPL | 457 | gnsr | 246 |
| | | | | | | | | | AR | | | |
| 18384.1 | −3.6 | 5.77 | 1148.646 | 0.00101 | 2 | 1 | ltfr | 448 | IPYYVGPL | 457 | gnsr | 246 |
| | | | | | | | | | AR | | | |
| 17313.1 | −3.2 | 6.29 | 1148.646 | −0.0007 | 2 | 1 | ltfr | 448 | IPYYVGPL | 457 | gnsr | 246 |
| | | | | | | | | | AR | | | |
| 20433.1 | −3 | 5.3 | 1148.646 | 0.00309 | 2 | 1 | ltfr | 448 | IPYYVGPL | 457 | gnsr | 246 |
| | | | | | | | | | AR | | | |
| 17728.1 | −3 | 5.9 | 1148.646 | −0.00118 | 2 | 1 | ltfr | 448 | IPYYVGPL | 457 | gnsr | 246 |
| | | | | | | | | | AR | | | |
| 18166.1 | −2.6 | 5.64 | 1148.646 | 1.003 | 2 | 1 | ltfr | 448 | IPYYVGPL | 457 | gnsr | 246 |
| | | | | | | | | | AR | | | |
| 19434.1 | −2.4 | 5.48 | 1148.646 | 0.00028 | 2 | 1 | ltfr | 448 | IPYYVGPL | 457 | gnsr | 246 |
| | | | | | | | | | AR | | | |
| 12617.1 | −2.5 | 6.39 | 843.3818 | −0.00118 | 2 | 1 | gnsr | 462 | FAWMTR | 467 | ksee | 247 |
| 19920.1 | −12.1 | 6.47 | 2051.981 | 0.02 | 2 | 0.667 | wmtr | 468 | KSEETITP | 484 | gasa | 248 |
| | | | | | | | | | WNFEEVVD | | | |
| | | | | | | | | | K | | | |
| 16230.1 | −6.7 | 5.91 | 2066.992 | 1.002 | 2 | 0.667 | wmtr | 468 | KSEETITP | 484 | gasa | 248 |
| | | | | | | | | | WNFEEVVD | | | |
| | | | | | | | | | K | | | |
| 19898.1 | −5.7 | 6.89 | 2050.997 | −0.00186 | 3 | 1 | wmtr | 468 | KSEETITP | 484 | gasa | 248 |
| | | | | | | | | | WNFEEVVD | | | |
| | | | | | | | | | K | | | |
| 19927.1 | −4.4 | 6.1 | 2066.992 | 1.001 | 3 | 1 | wmtr | 468 | KSEETITP | 484 | gasa | 248 |
| | | | | | | | | | WNFEEVVD | | | |
| | | | | | | | | | K | | | |
| 16287.1 | −4.1 | 6.22 | 2082.987 | −0.00354 | 3 | 1 | wmtr | 468 | KSEETITP | 484 | gasa | 248 |
| | | | | | | | | | WNFEEVVD | | | |
| | | | | | | | | | K | | | |
| 19949.1 | −4.1 | 6.42 | 2082.987 | 1.001 | 3 | 1 | wmtr | 468 | KSEETITP | 484 | gasa | 248 |
| | | | | | | | | | WNFEEVVD | | | |
| | | | | | | | | | K | | | |
| 18094.1 | −3.9 | 6.27 | 2082.987 | 1 | 3 | 1 | wmtr | 468 | KSEETITP | 484 | gasa | 248 |
| | | | | | | | | | WNFEEVVD | | | |
| | | | | | | | | | K | | | |
| 16199.1 | −3.8 | 6.29 | 2066.992 | 1 | 3 | 1 | wmtr | 468 | KSEETITP | 484 | gasa | 248 |
| | | | | | | | | | WNFEEVVD | | | |
| | | | | | | | | | K | | | |
| 20314.1 | −3.6 | 5.71 | 2050.997 | −0.00956 | 3 | 1 | wmtr | 468 | KSEETITP | 484 | gasa | 248 |
| | | | | | | | | | WNFEEVVD | | | |
| | | | | | | | | | K | | | |
| 20105.1 | −3.5 | 6.32 | 2050.997 | 0.00033 | 3 | 1 | wmtr | 468 | KSEETITP | 484 | gasa | 248 |
| | | | | | | | | | WNFEEVVD | | | |
| | | | | | | | | | K | | | |
| 17648.1 | −2.7 | 5.49 | 2082.987 | −0.00152 | 3 | 1 | wmtr | 468 | KSEETITP | 484 | gasa | 248 |
| | | | | | | | | | WNFEEVVD | | | |
| | | | | | | | | | K | | | |
| 19080.1 | −2.5 | 5.39 | 2066.992 | 1.001 | 2 | 0.667 | wmtr | 468 | KSEETITP | 484 | gasa | 248 |
| | | | | | | | | | WNFEEVVD | | | |
| | | | | | | | | | K | | | |
| 19028.1 | −2.5 | 5.69 | 2067.976 | 0.021 | 3 | 1 | wmtr | 468 | KSEETITP | 484 | gasa | 248 |
| | | | | | | | | | WNFEEVVD | | | |
| | | | | | | | | | K | | | |
| 17931.1 | −2.5 | 6.2 | 2066.992 | 1.004 | 3 | 1 | wmtr | 468 | KSEETITP | 484 | gasa | 248 |
| | | | | | | | | | WNFEEVVD | | | |
| | | | | | | | | | K | | | |
| 18098.1 | −2.4 | 5.42 | 2082.987 | 0.999 | 2 | 0.667 | wmtr | 468 | KSEETITP | 484 | gasa | 248 |
| | | | | | | | | | WNFEEVVD | | | |
| | | | | | | | | | K | | | |
| 17628.1 | −2.4 | 6.43 | 2066.992 | 1.005 | 3 | 1 | wmtr | 468 | KSEETITP | 484 | gasa | 248 |
| | | | | | | | | | WNFEEVVD | | | |
| | | | | | | | | | K | | | |
| 20388.1 | −3.6 | 5.43 | 2465.183 | 0.996 | 3 | 1 | wmtr | 468 | KSEETITP | 489 | sfie | 249 |
| | | | | | | | | | WNFEEVVD | | | |
| | | | | | | | | | KGASAQ | | | |
| 20436.1 | −3 | 5.65 | 3114.49 | 0.022 | 3 | 0.75 | wmtr | 468 | KSEETITP | 494 | mtnf | 250 |
| | | | | | | | | | WNFEEVVD | | | |
| | | | | | | | | | KGASAQSF | | | |
| | | | | | | | | | IER | | | |
| 21988.1 | −6.8 | 6.46 | 1922.902 | 1.004 | 2 | 1 | mtrk | 469 | SEETITPW | 484 | gasa | 251 |
| | | | | | | | | | NFEEVVDK | | | |
| 20039.1 | −5.5 | 6.04 | 1954.892 | 1.006 | 2 | 1 | mtrk | 469 | SEETITPW | 484 | gasa | 251 |
| | | | | | | | | | NFEEVVDK | | | |
| 18010.1 | −4.7 | 5.93 | 1938.897 | 1.004 | 3 | 1 | mtrk | 469 | SEETITPW | 484 | gasa | 251 |
| | | | | | | | | | NFEEVVDK | | | |
| 21860.1 | −4.3 | 5.83 | 1922.902 | 1.003 | 3 | 1 | mtrk | 469 | SEETITPW | 484 | gasa | 251 |
| | | | | | | | | | NFEEVVDK | | | |
| 19403.1 | −3 | 5.67 | 1938.897 | −0.001 | 3 | 1 | mtrk | 469 | SEETITPW | 484 | gasa | 251 |
| | | | | | | | | | NFEEVVDK | | | |
| 19838.1 | −2.2 | 5.63 | 1954.892 | 0.00355 | 2 | 1 | mtrk | 469 | SEETITPW | 484 | gasa | 251 |
| | | | | | | | | | NFEEVVDK | | | |
| 17179.1 | −7.6 | 6.26 | 2025.988 | 1.001 | 3 | 1 | itpw | 477 | NFEEVVDK | 494 | mtnf | 252 |
| | | | | | | | | | GASAQSFI | | | |
| | | | | | | | | | ER | | | |
| 11591.1 | −4.1 | 6.29 | 1065.532 | −0.00178 | 2 | 1 | vvdk | 485 | GASAQSFI | 494 | mtnf | 253 |
| | | | | | | | | | ER | | | |
| 12339.1 | −4 | 6.46 | 1066.516 | −0.00056 | 2 | 1 | vvdk | 485 | GASAQSFI | 494 | mtnf | 253 |
| | | | | | | | | | ER | | | |
| 11384.1 | −3.7 | 6.28 | 1065.532 | 0.00127 | 2 | 1 | vvdk | 485 | GASAQSFI | 494 | mtnf | 253 |
| | | | | | | | | | ER | | | |
| 9565.1 | −9.3 | 6.24 | 1466.694 | −0.00002 | 2 | 0.667 | fier | 495 | MTNFDKNL | 506 | vlpk | 254 |
| | | | | | | | | | PNEK | | | |
| 10615.1 | −6.5 | 5.64 | 1450.699 | −0.004 | 2 | 0.667 | fier | 495 | MTNFDKNL | 506 | vlpk | 254 |
| | | | | | | | | | PNEK | | | |
| 9391.1 | −3 | 5.52 | 1466.694 | 0.00052 | 3 | 1 | fier | 495 | MTNFDKNL | 506 | vlpk | 254 |
| | | | | | | | | | PNEK | | | |
| 10018.1 | −2.6 | 5.83 | 1466.694 | 0.982 | 3 | 1 | fier | 495 | MTNFDKNL | 506 | vlpk | 254 |
| | | | | | | | | | PNEK | | | |
| 7991.1 | −3.7 | 5.85 | 1104.568 | −0.00084 | 2 | 0.667 | rmtn | 498 | FDKNLPNE | 506 | vlpk | 255 |
| | | | | | | | | | K | | | |
| 25187.1 | −4.9 | 5.83 | 2020.006 | 1.002 | 3 | 1 | vlpk | 511 | HSLLYEYF | 526 | vkyv | 256 |
| | | | | | | | | | TVYNELTK | | | |
| 6012.1 | −2.1 | 6.52 | 871.3978 | −0.00062 | 2 | 1 | tkvk | 529 | YVTEGMR | 535 | kpaf | 257 |
| 8749.1 | −6.5 | 5.97 | 1104.605 | −0.00232 | 2 | 0.667 | egmr | 536 | KPAFLSGE | 545 | kaiv | 258 |
| | | | | | | | | | QK | | | |
| 8950.1 | −5 | 6.97 | 1104.605 | −0.00122 | 2 | 0.667 | egmr | 536 | KPAFLSGE | 545 | kaiv | 258 |
| | | | | | | | | | QK | | | |
| 8796.1 | −2.9 | 5.42 | 1104.605 | −0.002 | 1 | 0.333 | egmr | 536 | KPAFLSGE | 545 | kaiv | 258 |
| | | | | | | | | | QK | | | |
| 9421.1 | −2.5 | 5.57 | 1105.589 | −0.0033 | 2 | 0.667 | egmr | 536 | KPAFLSGE | 545 | kaiv | 258 |
| | | | | | | | | | QK | | | |
| 7261.1 | −3.2 | 5.57 | 1232.7 | −0.00052 | 3 | 0.75 | egmr | 536 | KPAFLSGE | 546 | aivd | 259 |
| | | | | | | | | | QKK | | | |
| 18503.1 | −4.8 | 5.76 | 1046.661 | −0.00063 | 2 | 0.667 | geqk | 546 | KAIVDLLF | 554 | tnrk | 260 |
| | | | | | | | | | K | | | |
| 18706.1 | −4.6 | 6.79 | 1046.661 | −0.00015 | 2 | 0.667 | geqk | 546 | KAIVDLLF | 554 | tnrk | 260 |
| | | | | | | | | | K | | | |
| 18918.1 | −3.8 | 5.74 | 1046.661 | 0.00107 | 2 | 0.667 | geqk | 546 | KAIVDLLF | 554 | tnrk | 260 |
| | | | | | | | | | K | | | |
| 21537.1 | −2.9 | 6.18 | 918.5659 | −0.00081 | 1 | 0.5 | eqkk | 547 | AIVDLLFK | 554 | tnrk | 261 |
| 21327.1 | −2.4 | 5.86 | 918.5659 | 0.00096 | 1 | 0.5 | eqkk | 547 | AIVDLLFK | 554 | tnrk | 261 |
| 22341.1 | −2.3 | 5.66 | 918.5659 | −0.00046 | 2 | 1 | eqkk | 547 | AIVDLLFK | 554 | tnrk | 261 |
| 14255.1 | −5.3 | 5.91 | 1053.525 | 0.00031 | 2 | 0.667 | vtvk | 563 | QLKEDYFK | 570 | kiec | 262 |
| 10536.1 | −2.7 | 6.96 | 1070.552 | −0.00024 | 2 | 0.667 | vtvk | 563 | QLKEDYFK | 570 | kiec | 262 |
| 11930.1 | −4.4 | 5.36 | 1181.62 | 0.00202 | 2 | 0.5 | vtvk | 563 | QLKEDYFK | 571 | iecf | 263 |
| | | | | | | | | | K | | | |
| 15209.1 | −8.2 | 5.73 | 1882.885 | 0.998 | 2 | 0.667 | dyfk | 571 | KIECFDSV | 586 | fnas | 264 |
| | | | | | | | | | EISGVEDR | | | |
| 15412.1 | −3.6 | 5.19 | 1882.885 | 1.008 | 2 | 0.667 | dyfk | 571 | KIECFDSV | 586 | fnas | 264 |
| | | | | | | | | | EISGVEDR | | | |
| 14914.1 | −3.6 | 5.56 | 1882.885 | −0.00025 | 3 | 1 | dyfk | 571 | KIECFDSV | 586 | fnas | 264 |
| | | | | | | | | | EISGVEDR | | | |
| 15188.1 | −3.1 | 5.93 | 1882.885 | 1.008 | 3 | 1 | dyfk | 571 | KIECFDSV | 586 | fnas | 264 |
| | | | | | | | | | EISGVEDR | | | |
| 15402.1 | −2.7 | 5.88 | 1882.885 | 0.00048 | 3 | 1 | dyfk | 571 | KIECFDSV | 586 | fnas | 264 |
| | | | | | | | | | EISGVEDR | | | |
| 16599.1 | −6.4 | 6.34 | 1754.79 | 0.999 | 2 | 1 | yfkk | 572 | IECFDSVE | 586 | fnas | 265 |
| | | | | | | | | | ISGVEDR | | | |
| 14060.1 | −2.6 | 6 | 1352.633 | 0.00038 | 2 | 1 | kiec | 575 | FDSVEISG | 586 | fnas | 266 |
| | | | | | | | | | VEDR | | | |
| 10394.1 | −2.2 | 5.85 | 1090.537 | 0.00504 | 2 | 1 | ecfd | 577 | SVEISGVE | 586 | fnas | 267 |
| | | | | | | | | | DR | | | |
| 16338.1 | −11.3 | 6.61 | 1478.764 | −0.00069 | 2 | 0.667 | vedr | 587 | FNASLGTY | 599 | iikd | 268 |
| | | | | | | | | | HDLLK | | | |
| 16334.1 | −6 | 6.75 | 1478.764 | 0.00033 | 3 | 1 | vedr | 587 | FNASLGTY | 599 | iikd | 268 |
| | | | | | | | | | HDLLK | | | |
| 16542.1 | −5.1 | 6.38 | 1478.764 | 0.00114 | 2 | 0.667 | vedr | 587 | FNASLGTY | 599 | iikd | 268 |
| | | | | | | | | | HDLLK | | | |
| 16541.1 | −3.9 | 6.7 | 1478.764 | −0.00031 | 3 | 1 | vedr | 587 | FNASLGTY | 599 | iikd | 268 |
| | | | | | | | | | HDLLK | | | |
| 17311.1 | −3.3 | 6.53 | 1479.748 | −0.00013 | 3 | 1 | vedr | 587 | FNASLGTY | 599 | iikd | 268 |
| | | | | | | | | | HDLLK | | | |
| 16424.1 | −2.4 | 5.75 | 1217.652 | −0.003 | 2 | 0.667 | drfn | 589 | ASLGTYHD | 599 | iikd | 269 |
| | | | | | | | | | LLK | | | |
| 33735.1 | −6.2 | 6.06 | 3253.563 | 1.001 | 3 | 1 | kiik | 603 | DKDFLDNE | 629 | emie | 270 |
| | | | | | | | | | ENEDILED | | | |
| | | | | | | | | | IVLTLTLF | | | |
| | | | | | | | | | EDR | | | |
| 34024.1 | −7.6 | 5.51 | 3011.426 | 1.027 | 3 | 1.5 | ikdk | 605 | DFLDNEEN | 629 | emie | 271 |
| | | | | | | | | | EDILEDIV | | | |
| | | | | | | | | | LTLTLFED | | | |
| | | | | | | | | | R | | | |
| 12463.1 | −2.6 | 5.99 | 1350.705 | −0.00093 | 3 | 0.75 | ieer | 636 | LKTYAHLF | 646 | vmkq | 272 |
| | | | | | | | | | DDK | | | |
| 11560.1 | −10.4 | 6.5 | 1483.725 | −0.0019 | 2 | 0.5 | erlk | 638 | TYAHLFDD | 649 | qlkr | 273 |
| | | | | | | | | | KVMK | | | |
| 12031.1 | −5.2 | 5.72 | 1499.72 | 1.002 | 2 | 0.5 | erlk | 638 | TYAHLFDD | 649 | qlkr | 273 |
| | | | | | | | | | KVMK | | | |
| 8153.1 | −2.7 | 5.83 | 895.4533 | −0.00184 | 2 | 0.667 | lkrr | 655 | RYTGWGR | 661 | lsrk | 274 |
| 21061.1 | −2.5 | 6.44 | 849.508 | −0.00076 | 1 | 0.5 | qsgk | 678 | TILDFLK | 684 | sdgf | 275 |
| 21968.1 | −2.4 | 5.78 | 1596.838 | 0.0013 | 3 | 1 | qsgk | 678 | TILDFLKS | 691 | nfmq | 276 |
| | | | | | | | | | DGFANR | | | |
| 17674.1 | −3.1 | 6.36 | 1449.668 | −0.00008 | 2 | 1 | fanr | 692 | NFMQLIHD | 703 | fked | 277 |
| | | | | | | | | | DSLT | | | |
| 17884.1 | −2.9 | 6.34 | 1449.668 | −0.00142 | 2 | 1 | fanr | 692 | NFMQLIHD | 703 | fked | 277 |
| | | | | | | | | | DSLT | | | |
| 18937.1 | −6.9 | 5.72 | 1724.831 | −0.00086 | 2 | 0.667 | fanr | 692 | NFMQLIHD | 705 | ediq | 278 |
| | | | | | | | | | DSLTFK | | | |
| 18733.1 | −5.7 | 5.78 | 1724.831 | 0.996 | 2 | 0.667 | fanr | 692 | NFMQLIHD | 705 | ediq | 278 |
| | | | | | | | | | DSLTFK | | | |
| 18713.1 | −3.5 | 5.93 | 1724.831 | −0.00136 | 3 | 1 | fanr | 692 | NFMQLIHD | 705 | ediq | 278 |
| | | | | | | | | | DSLTFK | | | |
| 18915.1 | −2.7 | 6.75 | 1724.831 | 0.00175 | 3 | 1 | fanr | 692 | NFMQLIHD | 705 | ediq | 278 |
| | | | | | | | | | DSLTFK | | | |
| 19275.1 | −2.2 | 6.08 | 1740.826 | −0.00421 | 3 | 1 | fanr | 692 | NFMQLIHD | 705 | ediq | 278 |
| | | | | | | | | | DSLTFK | | | |
| 19409.1 | −8.8 | 5.83 | 2354.133 | 0.999 | 3 | 0.75 | fanr | 692 | NFMQLIHD | 710 | aqvs | 279 |
| | | | | | | | | | DSLTFKED | | | |
| | | | | | | | | | IQK | | | |
| 19143.1 | −4.6 | 6.57 | 2338.138 | 1.002 | 3 | 0.75 | fanr | 692 | NFMQLIHD | 710 | aqvs | 279 |
| | | | | | | | | | DSLTFKED | | | |
| | | | | | | | | | IQK | | | |
| 18931.1 | −2.3 | 5.75 | 2338.138 | 0.00455 | 3 | 0.75 | fanr | 692 | NFMQLIHD | 710 | aqvs | 279 |
| | | | | | | | | | DSLTFKED | | | |
| | | | | | | | | | IQK | | | |
| 9214.1 | −4.6 | 5.82 | 1548.74 | −0.00068 | 2 | 0.667 | diqk | 711 | AQVSGQGD | 725 | nlag | 280 |
| | | | | | | | | | SLHEHIA | | | |
| 9235.1 | −3.9 | 6.42 | 1548.74 | 0.00108 | 3 | 1 | diqk | 711 | AQVSGQGD | 725 | nlag | 280 |
| | | | | | | | | | SLHEHIA | | | |
| 13996.1 | −11.7 | 6.07 | 2400.227 | 1.004 | 2 | 0.5 | diqk | 711 | AQVSGQGD | 734 | kgil | 281 |
| | | | | | | | | | SLHEHIAN | | | |
| | | | | | | | | | LAGSPAIK | | | |
| 13947.1 | −5.6 | 5.87 | 2401.211 | 1.024 | 3 | 0.75 | diqk | 711 | AQVSGQGD | 734 | kgil | 281 |
| | | | | | | | | | SLHEHIAN | | | |
| | | | | | | | | | LAGSPAIK | | | |
| 12640.1 | −3 | 5.79 | 2529.306 | 1.022 | 3 | 0.6 | diqk | 711 | AQVSGQGD | 735 | gilq | 282 |
| | | | | | | | | | SLHEHIAN | | | |
| | | | | | | | | | LAGSPAIK | | | |
| | | | | | | | | | K | | | |
| 21486.1 | −2.1 | 5.49 | 1540.931 | 1.004 | 3 | 1 | aikk | 736 | GILQTVKV | 749 | vmgr | 283 |
| | | | | | | | | | VDELVK | | | |
| 12080.1 | −4.3 | 6.04 | 1436.768 | −0.00192 | 2 | 0.5 | vmgr | 754 | HKPENIVI | 765 | enqt | 284 |
| | | | | | | | | | EMAR | | | |
| 8897.1 | −2.9 | 6.95 | 1452.763 | −0.00143 | 3 | 0.75 | vmgr | 754 | HKPENIVI | 765 | enqt | 284 |
| | | | | | | | | | EMAR | | | |
| 12055.1 | −2.4 | 6.68 | 1436.768 | −0.00252 | 3 | 0.75 | vmgr | 754 | HKPENIVI | 765 | enqt | 284 |
| | | | | | | | | | EMAR | | | |
| 9579.1 | −2.3 | 6.45 | 1468.758 | −0.00043 | 3 | 0.75 | vmgr | 754 | HKPENIVI | 765 | enqt | 284 |
| | | | | | | | | | EMAR | | | |
| 10031.1 | −2.7 | 5.94 | 1315.704 | 0.0023 | 3 | 1 | mgrh | 755 | KPENIVIE | 765 | enqt | 285 |
| | | | | | | | | | MAR | | | |
| 18986.1 | −2.8 | 5.78 | 1428.794 | −0.00007 | 2 | 1 | rmkr | 784 | IEEGIKEL | 796 | kehp | 286 |
| | | | | | | | | | GSQIL | | | |
| 16938.1 | −13.6 | 6.18 | 1556.889 | 0.00006 | 2 | 0.667 | rmkr | 784 | IEEGIKEL | 797 | ehpv | 287 |
| | | | | | | | | | GSQILK | | | |
| 17747.1 | −5.3 | 5.81 | 1557.873 | 1.002 | 2 | 0.667 | rmkr | 784 | IEEGIKEL | 797 | ehpv | 287 |
| | | | | | | | | | GSQILK | | | |
| 17145.1 | −4.8 | 5.68 | 1556.889 | 1.005 | 2 | 0.667 | rmkr | 784 | IEEGIKEL | 797 | ehpv | 287 |
| | | | | | | | | | GSQILK | | | |
| 17731.1 | −3.7 | 6.33 | 1557.873 | 1.002 | 3 | 1 | rmkr | 784 | IEEGIKEL | 797 | ehpv | 287 |
| | | | | | | | | | GSQILK | | | |
| 16937.1 | −3.1 | 6.66 | 1556.889 | 0.00035 | 3 | 1 | rmkr | 784 | IEEGIKEL | 797 | ehpv | 287 |
| | | | | | | | | | GSQILK | | | |
| 17144.1 | −2.2 | 6.37 | 1556.889 | 0.00017 | 3 | 1 | rmkr | 784 | IEEGIKEL | 797 | ehpv | 287 |
| | | | | | | | | | GSQILK | | | |
| 7132.1 | −3.1 | 6.97 | 1565.755 | −0.00264 | 3 | 1 | qilk | 798 | EHPVENTQ | 810 | lyly | 288 |
| | | | | | | | | | LQNEK | | | |
| 7452.1 | −2.6 | 5.19 | 1567.723 | 0.018 | 3 | 1 | qilk | 798 | EHPVENTQ | 810 | lyly | 288 |
| | | | | | | | | | LQNEK | | | |
| 7496.1 | −2.4 | 5.55 | 1566.739 | −0.00245 | 2 | 0.667 | qilk | 798 | EHPVENTQ | 810 | lyly | 288 |
| | | | | | | | | | LQNEK | | | |
| 7496.2 | −2.4 | 5.55 | 1566.739 | −0.00245 | 2 | 0.667 | qilk | 798 | EHPVENTQ | 810 | lyly | 288 |
| | | | | | | | | | LQNEK | | | |
| 7742.1 | −2 | 5.47 | 1566.739 | −0.00118 | 3 | 1 | qilk | 798 | EHPVENTQ | 810 | lyly | 288 |
| | | | | | | | | | LQNEK | | | |
| 18191.1 | −6.8 | 6.66 | 1302.684 | 0.00101 | 2 | 1 | qnek | 811 | LYLYYLQN | 820 | dmyv | 289 |
| | | | | | | | | | GR | | | |
| 17984.1 | −5.9 | 7.22 | 1302.684 | 0.00162 | 2 | 1 | qnek | 811 | LYLYYLQN | 820 | dmyv | 289 |
| | | | | | | | | | GR | | | |
| 18707.1 | −5.6 | 6.85 | 1303.668 | −0.00009 | 2 | 1 | qnek | 811 | LYLYYLQN | 820 | dmyv | 289 |
| | | | | | | | | | GR | | | |
| 18499.1 | −4.2 | 7.7 | 1303.668 | −0.00119 | 2 | 1 | qnek | 811 | LYLYYLQN | 820 | dmyv | 289 |
| | | | | | | | | | GR | | | |
| 18921.1 | −3.4 | 5.84 | 1303.668 | 0.00674 | 2 | 1 | qnek | 811 | LYLYYLQN | 820 | dmyv | 289 |
| | | | | | | | | | GR | | | |
| 15229.1 | −3 | 5.54 | 2258.051 | 0.985 | 3 | 1 | lyly | 815 | YLQNGRDM | 832 | lsdy | 290 |
| | | | | | | | | | YVDQELDI | | | |
| | | | | | | | | | NR | | | |
| 16141.1 | −7.9 | 6.44 | 1510.684 | −0.00132 | 2 | 1 | qngr | 821 | DMYVDQEL | 832 | lsdy | 291 |
| | | | | | | | | | DINR | | | |
| 16191.1 | −7.4 | 5.67 | 1526.679 | −0.00173 | 2 | 1 | ubgr | 821 | DMYVDQEL | 832 | lsdy | 291 |
| | | | | | | | | | DINR | | | |
| 14605.1 | −6.5 | 5.87 | 1526.679 | −0.001 | 2 | 1 | qngr | 821 | DMYVDQEL | 832 | lsdy | 291 |
| | | | | | | | | | DINR | | | |
| 14389.1 | −6.4 | 6.5 | 1526.679 | −0.00148 | 2 | 1 | qngr | 821 | DMYVDQEL | 832 | lsdy | 291 |
| | | | | | | | | | DINR | | | |
| 14184.1 | −6.3 | 6.04 | 1526.679 | 0.00096 | 2 | 1 | qngr | 821 | DMYVDQEL | 832 | lsdy | 291 |
| | | | | | | | | | DINR | | | |
| 14877.1 | −5.6 | 5.76 | 1542.674 | −0.00201 | 2 | 1 | qngr | 821 | DMYVDQEL | 832 | lsdy | 291 |
| | | | | | | | | | DINR | | | |
| 14254.1 | −5.3 | 6.23 | 1526.679 | 0.00003 | 3 | 1.5 | qngr | 821 | DMYVDQEL | 832 | lsdy | 291 |
| | | | | | | | | | DINR | | | |
| 14286.1 | −4.8 | 5.93 | 1510.684 | 1.988 | 2 | 1 | qngr | 821 | DMYVDQEL | 832 | lsdy | 291 |
| | | | | | | | | | DINR | | | |
| 14818.1 | −4.4 | 6.18 | 1527.663 | −0.00027 | 2 | 1 | ubgr | 821 | DMYVDQEL | 832 | lsdy | 291 |
| | | | | | | | | | DINR | | | |
| 14227.1 | −4.1 | 6.15 | 1542.674 | −0.00311 | 2 | 1 | qngr | 821 | DMYVDQEL | 832 | lsdy | 291 |
| | | | | | | | | | DINR | | | |
| 15755.1 | −4.5 | 6.93 | 1400.669 | −0.00073 | 2 | 1 | dinr | 833 | LSDYDVDH | 844 | sflk | 292 |
| | | | | | | | | | IVPQ | | | |
| 15360.1 | −4.9 | 6.04 | 1487.701 | 0.00008 | 2 | 1 | dinr | 833 | LSDYDVDH | 845 | flkd | 293 |
| | | | | | | | | | IVPQS | | | |
| 19358.1 | −6.4 | 6.17 | 1634.77 | 0.00137 | 2 | 1 | dinr | 833 | LSDYDVDH | 846 | lkdd | 294 |
| | | | | | | | | | IVPQSF | | | |
| 19720.1 | −8 | 6.72 | 1875.949 | 0.00159 | 3 | 1 | dinr | 833 | LSDYDVDH | 848 | ddsi | 295 |
| | | | | | | | | | IVPQSFLK | | | |
| 20138.1 | −6.1 | 6.08 | 1876.933 | 0.00103 | 3 | 1 | dinr | 833 | LSDYDVDH | 848 | ddsi | 295 |
| | | | | | | | | | IVPQSFLK | | | |
| 19731.1 | −5.3 | 5.77 | 1875.949 | 1.004 | 2 | 0.667 | dinr | 833 | LSDYDVDH | 848 | ddsi | 295 |
| | | | | | | | | | IVPQSFLK | | | |
| 19929.1 | −4.7 | 6.07 | 1875.949 | 0.00104 | 3 | 1 | dinr | 833 | LSDYDVDH | 848 | ddsi | 295 |
| | | | | | | | | | IVPQSFLK | | | |
| 19514.1 | −4.4 | 5.76 | 1875.949 | −0.00079 | 3 | 1 | dinr | 833 | LSDYDVDH | 848 | ddsi | 295 |
| | | | | | | | | | IVPQSFLK | | | |
| 18116.1 | −4.1 | 6.16 | 1875.949 | −0.00079 | 3 | 1 | dinr | 833 | LSDYDVDH | 848 | ddsi | 295 |
| | | | | | | | | | IVPQSFLK | | | |
| 19109.1 | −3.8 | 5.7 | 2664.268 | 0.02 | 3 | 0.75 | dinr | 833 | LSDYDVDH | 855 | vltr | 296 |
| | | | | | | | | | IVPQSFLK | | | |
| | | | | | | | | | DDSIDNK | | | |
| 19055.1 | −3.1 | 5.51 | 2664.268 | 0.019 | 2 | 0.5 | dinr | 833 | LSDYDVDH | 855 | vltr | 296 |
| | | | | | | | | | IVPQSFLK | | | |
| | | | | | | | | | DDSIDNK | | | |
| 18902.1 | −2.7 | 5.38 | 2664.268 | 0.024 | 3 | 0.75 | dinr | 833 | LSDYDVDH | 855 | vltr | 296 |
| | | | | | | | | | IVPQSFLK | | | |
| | | | | | | | | | DDSIDNK | | | |
| 10833.1 | −8.8 | 6.24 | 1281.632 | −0.00182 | 2 | 0.667 | ivpq | 845 | SFLKDDSI | 855 | vltr | 297 |
| | | | | | | | | | DNK | | | |
| 10302.1 | −4.1 | 5.47 | 1275.654 | 0.00055 | 2 | 0.667 | sflk | 849 | DDSIDNKV | 859 | sdkn | 298 |
| | | | | | | | | | LTR | | | |
| 10313.1 | −3.9 | 5.78 | 1275.654 | 0.00002 | 3 | 1 | sflk | 849 | DDSIDNKV | 859 | sdkn | 298 |
| | | | | | | | | | LTR | | | |
| 11162.1 | −3.1 | 5.36 | 1276.638 | −0.00141 | 2 | 0.667 | sflk | 849 | DDSIDNKV | 859 | sdkn | 298 |
| | | | | | | | | | LTR | | | |
| 9957.1 | −2.4 | 5.48 | 1275.654 | 0.00031 | 2 | 0.667 | sflk | 849 | DDSIDNKV | 859 | sdkn | 298 |
| | | | | | | | | | LTR | | | |
| 8324.1 | −3.6 | 6.7 | 1387.706 | −0.00065 | 3 | 1 | dknr | 865 | GKSDNVPS | 877 | kmkn | 299 |
| | | | | | | | | | EEVVK | | | |
| 9544.1 | −4.6 | 6.29 | 1202.59 | 0.0001 | 2 | 1 | nrgk | 867 | SDNVPSEE | 877 | kmkn | 300 |
| | | | | | | | | | VVK | | | |
| 7360.1 | −2 | 6.46 | 865.4778 | −0.0002 | 2 | 0.667 | itqr | 896 | KFDNLTK | 902 | aerg | 301 |
| 16484.1 | −3.4 | 5.81 | 1093.552 | 0.00171 | 2 | 1 | kaer | 906 | GGLSELDK | 916 | ikrq | 302 |
| | | | | | | | | | AGF | | | |
| 15744.1 | −6.7 | 6.35 | 1334.731 | −0.00068 | 2 | 0.667 | kaer | 906 | GGLSELDK | 918 | rqlV | 303 |
| | | | | | | | | | AGFIK | | | |
| 9145.1 | −5.2 | 6.43 | 1038.569 | 0.00069 | 2 | 0.667 | qitk | 930 | HVAQILDS | 938 | mntk | 304 |
| | | | | | | | | | R | | | |
| 9354.1 | −3.7 | 6.25 | 1038.569 | 0.00008 | 2 | 0.667 | qitk | 930 | HVAQILDS | 938 | mntk | 304 |
| | | | | | | | | | R | | | |
| 9698.1 | −3.5 | 6.32 | 1039.553 | −0.00079 | 2 | 0.667 | qitk | 930 | HVAQILDS | 938 | mntk | 304 |
| | | | | | | | | | R | | | |
| 10167.1 | −4.1 | 5.82 | 901.5102 | −0.00034 | 2 | 1 | itkh | 931 | VAQILDSR | 938 | mntk | 305 |
| 9624.1 | −3.6 | 5.44 | 1639.811 | 1.003 | 3 | 0.75 | ldsr | 939 | MNTKYDEN | 951 | evkv | 306 |
| | | | | | | | | | DKLIR | | | |
| 8536.1 | −3.5 | 5.42 | 1655.806 | −0.00149 | 2 | 0.5 | ldsr | 939 | MNTKYDEN | 951 | evkv | 306 |
| | | | | | | | | | DKLIR | | | |
| 8519.1 | −2.5 | 5.42 | 1655.806 | 0.00032 | 3 | 0.75 | ldsr | 939 | MNTKYDEN | 951 | evkv | 306 |
| | | | | | | | | | DKLIR | | | |
| 9408.1 | −8 | 5.44 | 1166.569 | −0.00295 | 2 | 0.667 | mntk | 943 | YDENDKLI | 951 | evkv | 307 |
| | | | | | | | | | R | | | |
| 9022.1 | −6.3 | 6.12 | 1165.585 | −0.00208 | 2 | 0.667 | mntk | 943 | YDENDKLI | 951 | evkv | 307 |
| | | | | | | | | | R | | | |
| 16391.1 | −11.9 | 6.47 | 2564.289 | 1.019 | 3 | 0.6 | ykvr | 977 | EINNYHHA | 999 | kypk | 308 |
| | | | | | | | | | HDAYLNAV | | | |
| | | | | | | | | | VGTALIK | | | |
| 20859.1 | −3.6 | 5.56 | 1447.816 | 0.00156 | 2 | 1 | hhah | 986 | DAYLNAVV | 999 | kypk | 309 |
| | | | | | | | | | GTALIK | | | |
| 15468.1 | −4.3 | 5.88 | 1349.626 | 0.00042 | 2 | 1 | kypk | 1004 | LESEFVYG | 1014 | vydv | 310 |
| | | | | | | | | | DYK | | | |
| 15259.1 | −4.2 | 6.34 | 1349.626 | −0.00129 | 2 | 1 | kypk | 1004 | LESEFVYG | 1014 | vydv | 310 |
| | | | | | | | | | DYK | | | |
| 15116.1 | −3.7 | 6 | 1349.626 | −0.00036 | 1 | 0.5 | kypk | 1004 | LESEFVYG | 1014 | vydv | 310 |
| | | | | | | | | | DYK | | | |
| 15050.1 | −2.3 | 6.34 | 1349.626 | 1.002 | 2 | 1 | kypk | 1004 | LESEFVYG | 1014 | vydv | 310 |
| | | | | | | | | | DYK | | | |
| 24923.1 | −2.7 | 6.1 | 1637.734 | 0.017 | 2 | 1 | atak | 1036 | YFFYSNIM | 1047 | teit | 311 |
| | | | | | | | | | NFFK | | | |
| 13762.1 | −4.7 | 7.06 | 1217.637 | 0.00042 | 2 | 1 | nffk | 1048 | TEITLANG | 1058 | krpl | 312 |
| | | | | | | | | | EIR | | | |
| 14466.1 | −3.9 | 6.1 | 1217.637 | 0.00152 | 2 | 1 | nffk | 1048 | TEITLANG | 1058 | krpl | 213 |
| | | | | | | | | | EIR | | | |
| 11197.1 | −7.7 | 6.32 | 1344.748 | −0.00018 | 2 | 0.667 | nffk | 1048 | TEITLANG | 1059 | rpli | 313 |
| | | | | | | | | | EIRK | | | |
| 11433.1 | −2.4 | 5.91 | 1345.732 | 0.00026 | 3 | 1 | nffk | 1048 | TEITLANG | 1059 | rpli | 313 |
| | | | | | | | | | EIRK | | | |
| 14337.1 | −11.6 | 6.05 | 2085.097 | 1.002 | 3 | 0.75 | geir | 1059 | KRPLIETN | 1076 | grdf | 314 |
| | | | | | | | | | GETGEIVW | | | |
| | | | | | | | | | DK | | | |
| 14832.1 | −10.4 | 6.28 | 2086.082 | 1.006 | 3 | 0.75 | geir | 1059 | KRPLIETN | 1076 | grdf | 314 |
| | | | | | | | | | GETGEIVW | | | |
| | | | | | | | | | DK | | | |
| 13567.1 | −6.5 | 6 | 2117.087 | 1.003 | 3 | 0.75 | geir | 1059 | KRPLIETN | 1076 | grdf | 314 |
| | | | | | | | | | GETGEIVW | | | |
| | | | | | | | | | DK | | | |
| 14082.1 | −2.5 | 5.73 | 2117.087 | 0.979 | 3 | 0.75 | geir | 1059 | KRPLIETN | 1076 | grdf | 314 |
| | | | | | | | | | GETGEIVW | | | |
| | | | | | | | | | DK | | | |
| 16689.1 | −3.5 | 5.95 | 1957.987 | 2.004 | 3 | 1 | eirk | 1060 | RPLIETNG | 1076 | grdf | 315 |
| | | | | | | | | | ETGEIVWD | | | |
| | | | | | | | | | K | | | |
| 16223.1 | −2.8 | 5.65 | 1957.003 | 0.00131 | 2 | 0.667 | eirk | 1060 | RPLIETNG | 1076 | grdf | 315 |
| | | | | | | | | | ETGEIVWD | | | |
| | | | | | | | | | K | | | |
| 14836.1 | −4.9 | 5.55 | 2170.125 | 1.004 | 3 | 0.75 | eirk | 1060 | RPLIETNG | 1078 | dfat | 316 |
| | | | | | | | | | ETGEIVWD | | | |
| | | | | | | | | | KGR | | | |
| 7593.1 | −3.1 | 6.06 | 921.4901 | −0.00121 | 2 | 0.667 | vwdk | 1077 | GRDFATVR | 1084 | kvls | 317 |
| 14448.1 | −2.6 | 6 | 1243.708 | 0.00104 | 2 | 1 | atvr | 1085 | KVLSMPQV | 1095 | kkte | 318 |
| | | | | | | | | | NIV | | | |
| 11613.1 | −5.4 | 6.09 | 1372.787 | 0.019 | 2 | 0.667 | atvr | 1085 | KVLSMPQV | 1096 | ktev | 319 |
| | | | | | | | | | NIVK | | | |
| 15190.1 | −2.4 | 5.79 | 1355.808 | 0.00012 | 2 | 0.667 | atvr | 1085 | KVLSMPQV | 1096 | ktev | 319 |
| | | | | | | | | | NIVK | | | |
| 11690.1 | −2 | 6.35 | 1371.803 | 0.00107 | 3 | 1 | atvr | 1085 | KVLSMPQV | 1096 | ktev | 319 |
| | | | | | | | | | NIVK | | | |
| 13300.1 | −3.3 | 7.09 | 1243.708 | −0.00066 | 2 | 1 | tvrk | 1086 | VLSMPQVN | 1096 | ktev | 320 |
| | | | | | | | | | IVK | | | |
| 14251.1 | −2.7 | 6.14 | 1259.703 | 0.00039 | 2 | 1 | tvrk | 1086 | VLSMPQVN | 1096 | ktev | 320 |
| | | | | | | | | | IVK | | | |
| 13786.1 | −2.1 | 5.96 | 1244.692 | 0.00031 | 2 | 1 | tvrk | 1086 | VLSMPQVN | 1096 | ktev | 320 |
| | | | | | | | | | IVK | | | |
| 7275.1 | −5.7 | 5.62 | 1181.616 | 0.00019 | 2 | 0.667 | nivk | 1097 | KTEVQTGG | 1107 | esil | 321 |
| | | | | | | | | | FSK | | | |
| 8438.1 | −2.6 | 5.86 | 1053.521 | −0.00324 | 2 | 1 | ivkk | 1098 | TEVQTGGF | 1107 | esil | 322 |
| | | | | | | | | | SK | | | |
| 11933.1 | −3.2 | 5.29 | 1739.856 | −0.00054 | 3 | 1 | ekgk | 1154 | SKESGSVS | 1169 | sldd | 323 |
| | | | | | | | | | SEQLAQFR | | | |
| 14805.1 | −2.3 | 6.02 | 1498.834 | 2.023 | 3 | 1.5 | sqvq | 1181 | LVQSGGGL | 1196 | lsca | 324 |
| | | | | | | | | | VQAGGSLR | | | |
| 19331.1 | −3.1 | 5.69 | 1458.618 | 0.00247 | 2 | 1 | fsgr | 1205 | TFSMYTMG | 1215 | qapg | 325 |
| | | | | | | | | | WFR | | | |
| 13115.1 | −7.1 | 6.11 | 1300.649 | −0.00202 | 2 | 1 | nrgr | 1232 | GLSPDIAD | 1244 | ftis | 326 |
| | | | | | | | | | SVNGR | | | |
| 18033.1 | −3.8 | 6.24 | 1227.6 | 0.00045 | 2 | 1 | klpk | 1342 | YSLFELEN | 1351 | krml | 327 |
| | | | | | | | | | GR | | | |
| 18711.1 | −2.6 | 5.81 | 1228.584 | −0.00028 | 2 | 1 | klpk | 1342 | YSLFELEN | 1351 | krml | 327 |
| | | | | | | | | | GR | | | |
| 21063.1 | −2.2 | 6.08 | 1646.821 | 0.00013 | 3 | 1 | lpsk | 1373 | YVNFLYLA | 1385 | lkgs | 328 |
| | | | | | | | | | SHYEK | | | |
| 12911.1 | −2.1 | 6.52 | 1071.604 | 0.00119 | 2 | 1 | fskr | 1421 | VILADANL | 1430 | vlsa | 329 |
| | | | | | | | | | DK | | | |
| 20865.1 | −3.8 | 5.75 | 2160.118 | 0.023 | 3 | 1 | tstk | 1482 | EVLDATLI | 1500 | idls | 330 |
| | | | | | | | | | HQSITGLY | | | |
| | | | | | | | | | ETR | | | |
| 14404.1 | −4.7 | 6.2 | 1286.659 | 1 | 2 | 1 | yetr | 1501 | IDLSQLGG | 1513 | kkrk | 331 |
| | | | | | | | | | DGSPK | | | |

sgRNA/gRNA Synthesis

sgRNA (Cas9 derivatives, Table 12) were either purchased as single piece guides from commercial suppliers or synthesised by in vitro synthesis (IVT) inhouse. The IVT synthesis method for synthesizing sgRNA includes the synthesis of a ssDNA of the following format (following the NEB sgRNA guide synthesis method):

A T7 polymerase promoter sequence followed by cr DNA sequence with overlap for reverse complement strand encoding the tr:RNA backbone as DNA.

A NEB™ EnGen™ synthesis kit was used for the IVT synthesis. The DNA strand was added to the pre mixed reaction mixture as per the manufacturer's instructions (recommended 2 micrograms of template DNA, of the form: T7 promoter-GG-XXXXXXXX seed sequence and backbone) and incubated for 12 h at 37° C. for maximum yield of a short template. RNA was confirmed using bleach agarose gel or urea polyacrylamide gel electrophoresis. RNA was cleaned of impurities using a Zymo™ clean and concentrate kit as per manufacturer's instruction. Quantification was performed by UV/VIS and RNas inhibitors were added (various manufacturers), before storing at −80° C.

Guide RNA for Cas12 were either purchased from Horizon™ or IDT™ as single piece guides or synthesised by overlap PCR to create a double stranded DNA template. The double stranded template contained a T7 promoter sequence followed by tr gRNA backbone for cas12a and terminated by the cr RNA (as DNA sequence for the guide). NEB™ T7 transcription kit was used to convert the sequence to RNA, with all subsequent steps of purification, quantification and storage being identical to those in the synthesis of the Cas9 derivative sgRNA guides as described above.

### T7 Endonuclease Assay for Gene Edit Evaluation

The principle of a T7 endonuclease I assay is to demonstrate indel formation in a gene edited locus. The first step was PCR amplification from extracted genomic DNA, followed by PCR amplicon purification. Following the NEB protocol, Amplicons are heated to 95° C. for 2-5 mins before being cooled gradually, to form a heteroduplex, between WT and Edited strands. This mismatch causes a bulge in the DNA that is recognised by T7 endonuclease I which cleaves the strands, incubation is generally at 37° C. for 20-30 mins. With removal of the endonuclease by 1 M EDTA or preferably proteinase K treatment for 5 mins at 56° C., the samples were ready for gel analysis upon a 1.5% agarose gel TAE buffer, run at 100 V for 20 mins.

The aim was to demonstrate the indel formation in a rapid and cost effective manner. Formation of a cleavaged product or it's degradation of the original amplicon if the indel formation is large it is a clear determinant of gene editing having been accomplished.

All T7 assays were preceded by DNA extraction from samples cells, using silica column DNA extraction and purification methods as per manufacturer's instructions (Biobasic Genomic DNA extraction kit) or alternatively using Thermofisher direct PCR with Protease K & detergent cell lysis.

TRAC specific guide RNA molecules used by M7 Cas12 derivative nucleases (e.g. C9m or Cas9) are presented in Table 12.

TABLE 12
Guide RNA sequences

| Guide | | | | SEQ |
|---|---|---|---|---|
| Name | pam | gRNA sequence | Location in sequence | ID NO |
| T1(−ve3) | TTTG | CCCCAACCCAGG | −71 from start of exon 1 | 332 |
| | | CTGGAGTCC | | |
| T2(−ve2) | TTTC | CCTCTTTGCCCC | −63 from start of exon 1 | 333 |
| | | AACCCAGGC | | |
| T3(+ve1) | CTTG | TCCCACAGATAT | +14 from start of exon 1 | 334 |
| | | CCAGAACCC | | |
| T4(-ve1) | TTTA | GAGTCTCTCAGC | +22 from start of exon 1 | 335 |
| | | TGGTACACG | | |

PCR amplification was performed using Kras G12s primers, primers also amplify WT Ras sequence and were used on DNA samples from A549 and H2228. For PCR amplification Thermofisher™ Direct PCR or KDpIus™ (transgen) could be used with either silica column purified DNA or direct PCR samples, reactions were set up as per each manufacturer's specifications and for primers temperature set was 58° C.

Amplicons can be used directly in T7 endonuclease assay but it is preferable to perform PCR clean up. Quantification of purified products was achieved by UV/VIS spectroscopy.

To set up the T7 reaction, 200 ng of PCR product, 2 μL of 10×NEB™ buffer 2.0, and H₂O was added up to a volume of 19 μL. The reaction was performed in 0.2 mL PCR tubes.

Annealing of PCR products was performed to form heteroduplexes, to do so a PCR thermocycler is used to perform the following steps:
Initial denaturation was performed at 95° C. for 5 minutes,
Annealing was performed at 95-85° C. with a temperature decline of 2° C./second until 85° C. and then at 0.1° C./second rate until 25° C.

T7 was added to the annealed DNA sample (1 μL of T7 endonuclease) and incubation was performed for 1 hr at 37° C. The reaction was stopped by addition of proteinase K and incubated at 37° C. for 20 mins, to remove T7 endonuclease from cleaved DNA products. With the addition of 4 μL of fluorescent DNA dye (sybr) the products were run on a 1.5 to 2% gel and imaged by Chemi Doc™

Fluorescent Labelling with pHab Dye

Fluorescent labeling was performed in order to visualize the localization, binding and cell internalization of the nucleases. pHab is a pH sensitive dye produced by Promega™ in both N-hydroxysuccimide ester (NHS) or maleimide formats for bioconjugation. Bioconjugation to PNME proteins was achieved by following the manufacturer's instructions for amide coupling of N-succinimide pHaB dye to primary amines on the proteins. In brief, protein (5-10 mg) is aliquoted to a 1.5 ml tube and dye is dissolved in DMSO (200 microliters per 1 mg), 24 microliters to provide at least a 5:1 excess dye:protein, dependent on protein molecular weight. Incubate the reaction mixture on ice for 4 hrs in light excluding conditions. Purification of protein from unconjugated dye involved a two step quenching of remaining NHS groups (either 1 M Tris or ethanolamine) and gel extraction of the remaining small molecules (G25 spin column Pierce). Purified protein was then tagged with a pH sensitive dye. When internalised into cells a decline in pH leads to increase in fluorescence. The protocol for addition of other NHS ester dyes such as Cy5.5 NHS or Tamra NHS was achieved with the same method.

### Generalised Fusion Protein Preparation

For a functioning CRISPR nuclease, ratios between 1:1 and 1:9 (Nuclease:sgRNA) can be used. Generally an equimolar formulation is appropriate if the protein is of good quality and was well stored. As an example 1 μM of nuclease protein was pipetted into a 0.2 mL polymerase chain reaction (PCR) tube and 1 μM guide RNA was added, with gentle pipette mixing. Complexation was completed at room temperature in 15 to 20 mins. Unless specified otherwise all conditions followed this method of sgRNA complexation.

When the nuclease was used in combination with either a biotinylated donor or biotinylated aptamer or biotinylated scFv or biotinylated peptide, the biotin modified component was added in an equimolar ratio to the protein complex.

### In Vitro Cleavage Protocol

The cleavage (i.e. the function of the nuclease) was first evaluated in vitro to confirm that the insertion of the display domain did not affect the nuclease cleavage function. The PNME and sgRNA/gRNA was first thawed on ice. The PCR product cleavage template (Kras g12s amplicon synthesised by PCR from A549 cells) was defrosted. A PCR composition as detailed in Table 13 was prepared and mixed by pipetting then incubated at 37° C. for 45 mins. To produce the PCR composition the gRNa and nuclease are first mixed in buffer, and allowed to complex for 20 mins, at room temperature, then the template is added.

TABLE 13
Composition of the PCR reaction mixture and sequences
PCR Composition

| H₂O | 14 μL | |
|---|---|---|
| 10x | 2 μL | |
| nuclease | | |
| buffer | | |
| 75 μM | 2 μL | |
| gRNA | | |
| 1 μM | 1 μL | |
| Md7 (50 | | |
| nMoles) | | |
| PCR | 1 μL (aiming for 0.1 μM or 5 nMoles) | |
| template | | |
| | | SEQ |
| Nucleic | | ID |
| acid | Sequence | NO: |
| Kras | GGTACTGGTGGAGTATTTGATAGTGTATTAACCTTATGTGTGACATGT | 336 |
| G12s | TCTAATATAGTCACATTTTCATTATTTTTATTATAAGGCCTGCTGAAA | |
| | ATGACTGAATATAAACTTGTGGTAGTTGGAGCTAGTGGCGTAGGCAAG | |
| | AGTGCCTTGACGATACAGCTAATTCAGAATCATTTTGTGGACGAATAT | |
| | GATCCAACAATAGAGGTAAATGTTGTTTTAATATGCATATTACTGGTG | |
| | CAGGACCATTCTTTGATACAGATAAAGG | |
| | (crDNA recognition underlined and | |
| | Kras G12s codon in bold) | |
| Forward | CTGGTGGAGTATTTGATAGTGTA | 337 |
| primer | | |
| Reverse | ATTCGTCCACAAAATGATTCTGA | 338 |
| primer | | |
| gRNA | TTCTAATACGACTCACTATAGGCTTGTGGTAGTTGGAGCTAGGTTTT | 339 |
| | AGAGCTAGA | |
| crRNA | CUUGUGGUAGUUGGAGCUAG | 340 |

A blank reaction was prepared as described above but without the guide, thus preventing the cleavage of the template. The template was added (and mixed by pipetting) to both the blank reaction and the test reaction (with guide). The resulting mixture was incubated in a thermocycler for 45 minutes at 37° C. 1 microliter of proteinase K (10-20 mg/mL) was added and mixed after incubation and was left to incubate at 37° C. for 15 minutes. A 4 μL loading of fluorescent DNA Dye (i.e. sybr) was added in all of the reaction into wells. The results were analysed by running a 1.5 to 2% agarose gel, in order to confirm cleavage. All reactions were run with a negative control to compare the template. The negative control did not include any nuclease which was substituted with an additional volume of H₂O.

The gel was used to calculated the guide efficiency (in vitro). Quantification was based on relative band intensities. Indel percentage was determined by the formula:

$$100 * ( 1 - \sqrt{1 - \frac{b + c}{a + b + c}} )$$

where a is the integrated intensity of the undigested PCR product, and b and c are the integrated intensities of each cleavage product.

### Biotinylated Donor Construct Synthesis

Biotinylated donor CAR DNA constructs were produced in the following manner. A double strand DNA construct was synthesized containing the key elements of the CAR construct, with LHA and RHA arms as detailed above. PCR primers were designed to provide equal length homology arms (LHA/RHA) for the gel retardation assay. Primers for full length 400 bp arms were used resulting in a product around 3167 bp and for cell assay 100 bp arm primers were used, where a small construct is preferred. In some conditions, biotin was introduced to the amplicon as a 5′ modification on the forward or reverse primer.

PCR was performed with a high fidelity polymerase (Kdplus), with appropriate primers (tm58° C.). The double strand template was kept below 1 ng total in reaction and amplified by PCR over 35 cycles. PCR reaction volume, primer concentration were used as per manufacturer's instructions (Transgen™ biotech). PCR products were confirmed by 1.5% agarose gel and purified by PCR clean up column (Favorgen™) prior to use in either gel retardation assay or cell evaluations. Quantification was performed using UV/Vis spectroscopy, and stored at −80° C. until required.

### Cell Methods

The cells used were either Jurkat-lucia immortalised T-cell (Invivogen™) referred to as Jurkat or primary CD4⁺ T-cells (HemaCare™) from a single donor. Cells were cultured at 37° C. and 5% CO₂ in Roswell Park Memorial Institute (RPMI) medium (10% FBS, Pen/strep with sodium pyruvate and glutamate). The media was purchased from Wisent™. The cultures were passaged when they reached a confluence of 90% or every third day.

For cell experiments to evaluate CAR insertion 50′000 Jurkat T-cells were plated in each well of a 6 well plate in 4 mL of Roswell Park Memorial Institute (RPMI) media 10% FBS supplemented with penicillin/streptomycin (pen/strep), glutamate and sodium pyruvate as a suspension culture. After 12 hrs after seeding in a 6 well plate, the nuclease was prepared. For each well the nuclease was prepared in the following manner: 5 μg of nuclease was complexed with equimolar sgRNA and donor DNA in a (1:1:1 ratio) for 15 mins. If performing generalised cationic delivery, 2.5 μg or RL peptide was added to the 0.2 mL tube and mixed well by pipetting. If using a receptor mediated formulation RL peptide was note added. After 20 mins, an additional 100 μL RPMI media was to the tube and introduced to each well and mixed with the media by gentle rotation. Cells were incubated at 37° C./5% CO₂ for 72 hrs. Suspension culture was sampled periodically for the next 72 hrs using fluorescent microscopy to observe green fluorescence protein (GFP) signal as an indication of successful integration. Cells sampled from media (200 μL volume, were spun down and fixed with 4% paraformaldehyde or ice cold 100% Methanol. After 10 mins cells were spun down, washed with ice cold phosphate buffed saline (PBS) and added drop wise on to a microscope slide, with the addition of an microscope slide fixant. A cover slip was added and microscope images were taken using brightfield and fluorescent microscopy.

TABLE 14
Biotinylation Primers for gel retardation assay

| Name | Sequence | SEQ ID NO |
|---|---|---|
| Car-Fw | 5′Biotin-AGTTTGCTTTGCTGGGCCTTT | 341 |
| Car-Rev | TGGCAATGGATAAGGCCGAGAC | 342 |

After 72 hrs cells were harvested by collection of the cell culture media, centrifugation at 500 rpm to pellet cells. The cell pellet was washed twice with ice cold PBS and prepared for DNA extraction.

TABLE 15
Biotinylation Primers for Cell assay

| Name | Sequence | SEQ ID NO |
|---|---|---|
| Car100FW | AGCCCCGCCCTTGTC | 343 |
| Car100Rev | ACATTTGTTTGAGAATCAA | 344 |
| | AATCGGTGAATAGG | |

### Gel Retardation Assay

The gel retardation assay was used to confirm the binding of biotinylated DNA donors with C9m derivative nucleases (C9mAur, C9C4 and other derivatives). To perform the assay, biotinylated donors prepared by PCR amplification were defrosted and a volume equivalent to 1 μM was added to a 0.2 mL PCR tube. To achieve a 1:1 ratio between protein and biotinylated donor, 1 μM of C9mAur derivative or M7Mav Derivative (containing MA domain to bind biotin) was added to the tube and the final volume was 20 μL. Control samples containing only donor were prepared to validate unbound DNA. Control and protein:DNA samples were incubated at room temperature for biotinylation to occur for a minimum of 15 minutes. 4 μL of fluorescent DNA dye was added to visualise the DNA component and samples were run on a 1.5% agarose gel at 100V for 20 mins. DNA was visualised by Biorad™ Chemi Doc, retardation of the DNA was observed via fluorescence in samples with protein containing MA domain and in its absence the DNA would run appropriately to its length in BP and was visualised with a standard 1 kb size marker (Transgen™ 1 kb-plus marker). Complete complexation in a 1:1 ratio occurred with all C9m and M7ma proteins. The complex was stable and MA was appropriately folded.

Gel retardation assays were performed by mixing MA protein constructs in equimolar concentration with a biotinylated donor construct and incubating for 15 mins (1 μM of each biomolecule in a 20 μL volume). Full complexation occurred within 15 mins at room temperature, at which point a non-toxic DNA dye (sybr) was added to the mixture to bind DNA. 10 microliters was sampled and run on a 1.5% agarose gel in Tris-acetate-EDTA (TAE) buffer at 100V. For 30 mins. Imaging was accomplished by with BioRad™ chemi doc.

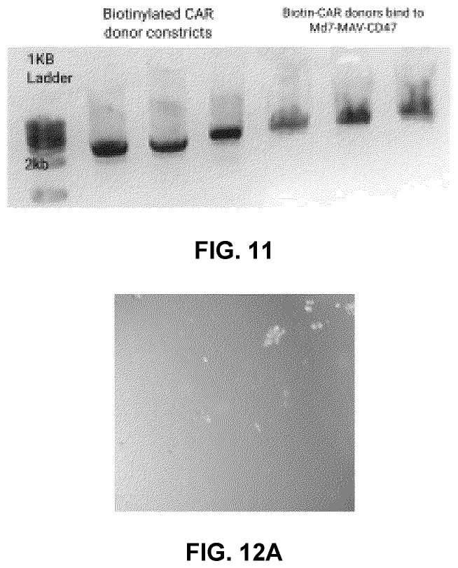

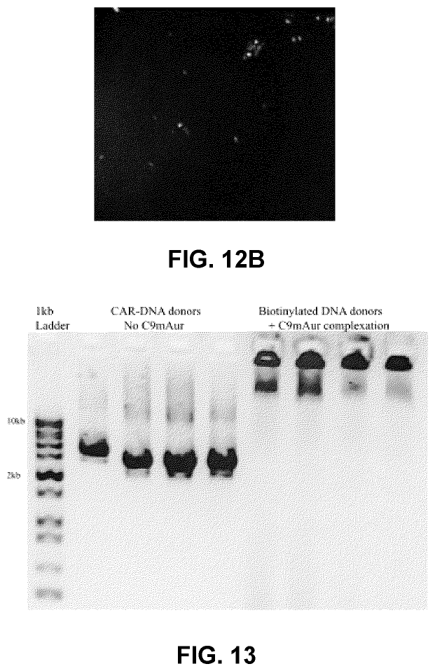

Initially M7MAV-CD8, and M7MAV-CD4 formed as a complexed protein and introduced to Jurkat cell culture in 6 well plates. For M7-Mav-CD4 and CD8 it was observed that CD4 modified construct was the only one to generate GFP signal whereas anti CD8 modified construct did not. Because Jurkat is a CD4 positive cell line, the editing resulting in GFP signal where an anti-CD4 M7MAv protein construct constitutes a demonstration of selectivity. It was later observed that extended GGGS linker domains in each constructs amino acid sequences lead to poor stability of the protein construct and attenuated translation in protein synthesis. The consequence of poor stability and maintenance of the fusion between MAV and nuclease domains can be seen in the limited gel retardation of complex (FIG. 11) and in the microscopy images of FIGS. 12A and 12B. Development of these constructs was discontinued in favour of the C9m derivatives which offered better stability. The improved gel retardation of DNA binding to MAV in C9m derived constructs encouraged further investigation.

Improvements to protein stability, endosomal escape and complexation with donor were hypothesised to improve overall CAR generation rate, these were implemented with the experiments with C9m derivatives and performed without the use of the GGGS linker, additional endosomal escape sequences and increased stability. To confirm improved stability of the MAV domain in complex with C9m derivatives, the gel retardation assay was performed again, with C9mAur derivative to affirm improved retardation of biotinylated donor. This time the donor is retained within gel wells, as the MW of the protein (˜185 kda) will not pass readily into the agarose gel (FIG. 13) showing the gel retardation example of complexation with donor (C9m)).

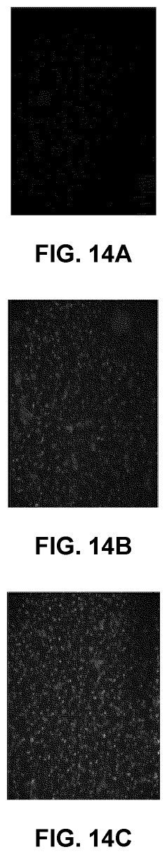

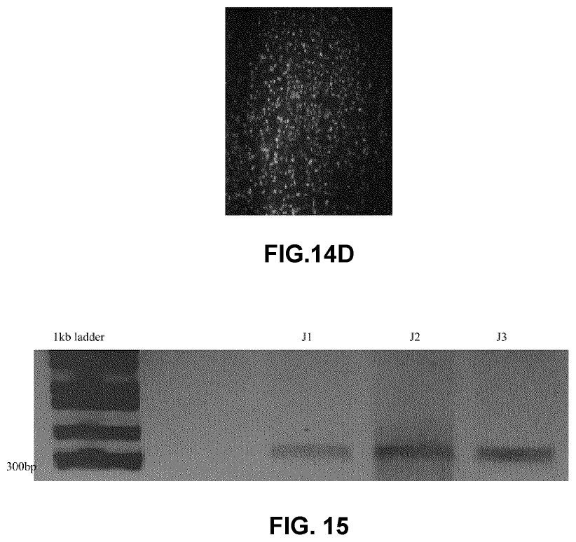

A GFP knock-in assessment was performed with donor construct genomic insertion. Three guide RNAs (sgRNA1, sgRNA3, and sgRNA11, Table 16) that closely group on TRAC exon 1 with non optimal donor fragment were used for the gene editing. The knock-in of the GFP was performed using the C9m derivative. The cells were harvested at the 24 hrs stage and fixed with paraformaldehyde before immobilization onto a glass slide to perform fluorescent microscopy imaging. It became clear that the increased stability was increasing the GFP signal and henced HDR gene editing, was observed from cells in greater number (FIGS. 14A-14D) that for M7MAVCD4. Significant GFP signal can only be produced if the CAR inserted has been inserted at the TRAC locus in frame with the endogenous promoter. If the CAR is present in the cell without insert, there is no potential signal as the donor construct lacks a promoter. From the imaging, (FIGS. 14A-14D) it was observed that around 80-90% of cells were producing a GFP signal (FIGS. 14B-14D) compared to no detected fluorescence in the control (FIG. 14A). Samples cells were taken for further analysis by PCR and NGS. It was determined from the results that sg11 is a preferred guide for CAR introduction.

TABLE 16
Cas9 derivative crRNA sequences

| Name | Sequence | SEQ ID NO |
|---|---|---|
| sgRNA11 (DNA) | ACAAAACTGTGCTAGACATG | 345 |
| sgRNA11 (RNA) | ACAAAACUGUGCUAGACAUG | 346 |
| sgRNA3 (DNA) | AACAAATGTGTCACAAAGTA | 347 |
| sgRNA3 (RNA) | AACAAAUGUGUCACAAGUA | 348 |
| sgRNA1 (DNA) | TCAGGGTTCTGGATATCTGT | 349 |
| sgRNA1 (RNA) | UCAGGGUUCUGGAUAUCUGU | 350 |

For further confirmation of edit formation and insertion into the genome the insertion site was evaluated and the CAR sequence was confirmed. A splice site analysis was performed, where one primer was targets the genomic TRAC target upstream of donor homology arms, and reverse primer targets the anti-CD19 scFV sequence of the CAR donor. The successful insertion was confirmed by PCR. FIG. 15 shows the gel run after the insert confirmation PCR. PCR was performed with the insert confirmation primers (Table 17).

TABLE 17
Insert Confirmation primers sequences

| Name | Sequence | SEQ ID NO |
|---|---|---|
| CWFw | GGGTTGGGGCAAAGAGG | 351 |
| CWRev | AGAGAGGCAGACAGGGAGG | 352 |

0.5 mL of media containing GFP expressing Jurkat cells was taken (3 biological replicates where sg11 guide was used). Cells were spun down at 500 rpm for 1 min and media was removed. Cells were lysed and the DNA was extracted by silica column (Biobasic™ One 4 all Genomic DNA extraction kit). Genomic DNA was amplified with the TRAC/CAR specific primers to confirm insertion. Insertion was confirmed and the amplicon was purified by PCR clean up column. Quantification was performed and samples normalised for amplicon sequence. Pair ended Amplicon sequencing inclusive of quantification, library preparation and insert analysis was performed by Genewiz™. It was observed that CAR had been successfully spliced into the TRAC genome, as it was observed that the splice site contained part TRAC locus sequence and the anti-CD19 scFV sequence of the insert. The consensus sequence of insert position in the TRAC exon1 and 5′ end of CAR constructs, is shown in the annotated SEQ ID NO:353 shown below. The genomic TRAC sequence is underlined, and the insertion break point is shown with a straight line.

SEQ ID NO: 353:
ATCTAAATCCAGTGACAAGTCTGTCTGCCTATTCACCGA
TTTTGATTCTCAAACAAATGTGTCACAAAGTAAGGATTC
TGATGTGTATATCACAGACAAAA|ATGCTCAGGCTGCTC
TTGGCTCTCAACTTATTCCCTTCAATTCAAGTAACAGGA
GGGTCTTCGACTACAAGGATCATGACGGAGACTATAAGGA

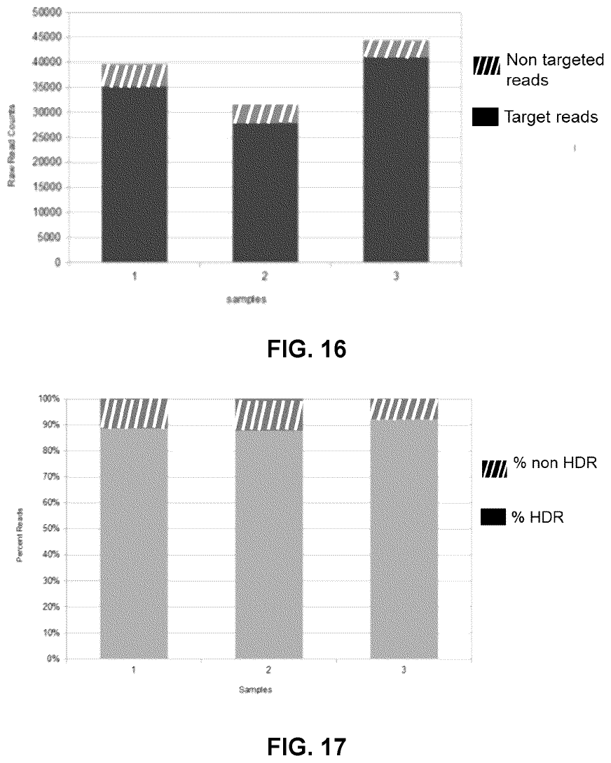

NGS sequencing enabled the estimation of the percentage CAR insertion to the genomes of the cell population subjected to gene editing. The correlation between NGS estimated HDR CAR insertion rate strongly correlated with GFP reporter signal expression of around 90% (FIGS. 16 and 17). More specifically, the HDR insertion of CAR was estimated at between 88 to 92% over 3 biological replicates.

Generalised Cationic Cell Delivery with RL Peptide

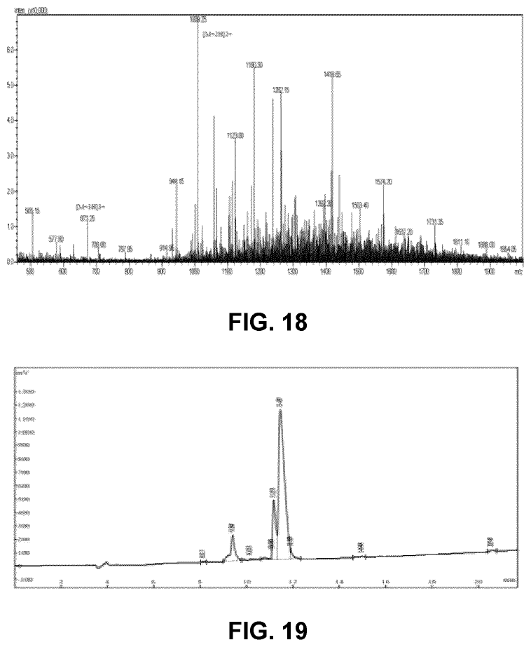

To perform cationic peptide based delivery, a peptide containing arginine and lysine repeats residues was used, namely the “RL” SEQ ID NO: 11: RRRRRRRLLLLLLLL. Peptide synthesis was completed by a commercial supplier (Biomatik™) and characterized by mass spectrometry (FIG. 18) to determine the correct synthesis of sequence. Purification was performed by high-performance liquid chromatography (HPLC) (FIG. 19) and lyophilized. Resuspension was performed at 1 mg/mL in MilliQ™ and stored at −80° C. until required. Complexation of the nuclease was accomplished by mixing the nuclease with biotinylated donor attached in a ratio of 5 μg of peptide to every 10 μg of protein used in nuclease formation. Peptide was added to the nuclease at room temperature and mixed by gentle pipetting. Complexation was complete after 15 mins and the complex was ready to be added to cells in appropriate concentration.

### Primary Cells

The following experiments were performed with human primary cells. Primary cells are important as they are a direct clinical analogue of the methods performance. Indeed, when performing cell therapy, primary cells recovered from a subject are the cells that will be genetically edited into CAR-T cells for treatment.

To allow rapid validation of the delivery of the modified nucleases (Zero, L1, L2 and L3), the modified nucleases were labeled with tetramethylrhodamine via the NHS amine coupling mechanism and purified by gel filtration. When complexed with a TRAC specific sgRNA of known performance, double strand breaks are generated by the intracellular delivery mediated by anti-CD4 binding capacity, endosomal escape capability and nuclear location of the modified nuclease constructs. In the HDR system required for CAR generation, the modified nuclease has the capacity to also bind a hapten modified donor DNA construct encoding the anti CD19 CAR with a P2A cleavage site followed by a GFP expression as a signal of successful insertion to genome.

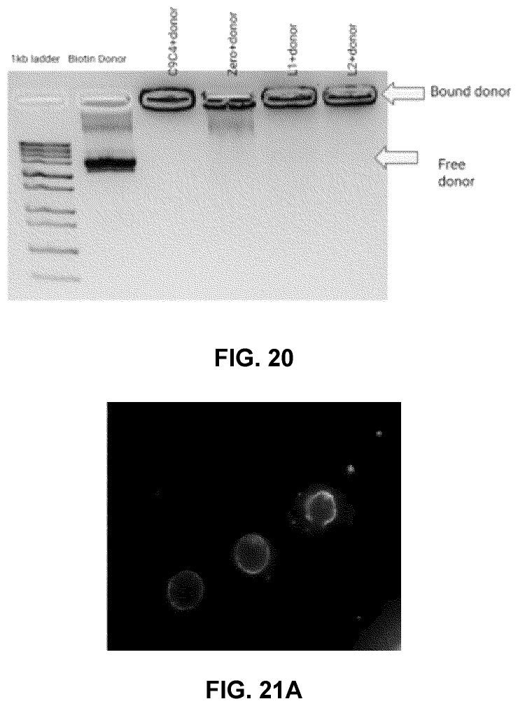

The modified nucleases (Zero, L1, L2, and L3) were subjected to a gel retardation assay of biotin DNA donor. More specifically, all the modified nucleases were subjected to QA biotin donor binding experiment. If donor DNA is bound by the modified nucleases, these will migrate slower in the gel and spend more time in the well (in other words they are retarded). FIG. 20 shows the gel electrophoresis for the gel retardation assay. As can be seen in FIG. 20, all modified nucleases were retarded and barely moved down the gel indicated that they bound the DNA donor by biotin interaction.

### Membrane Association in Primary T Cells

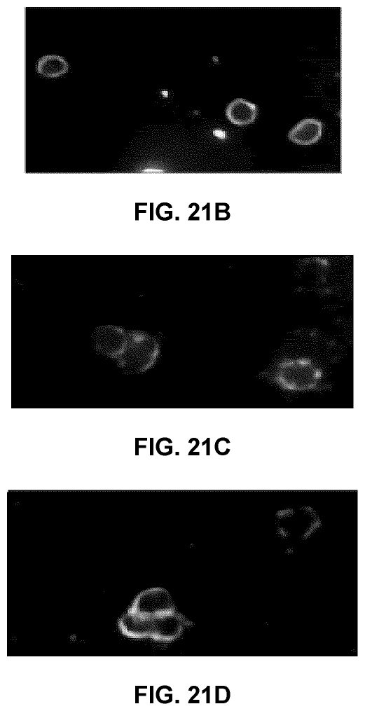

Fluorescent microscopy was performed to determine and visualize the association of modified nucleases (Zero, L1, L2, and L3) with the cell membrane of primary T-cells. All the modified nucleases were labelled with NHS-TAMRA fluorphore and purified by dye removal column. 5 μg of each modified nuclease was incubated with the CD4+ primary T-cells for 24 hours before fixation with formalin 5% in solution which was then transferred to a microscopy coverslip. Fluorescent microscopy at 40× oil immersion was accomplished using a BX51 and TAMRA filter set (ex 550 nm em 578-600 nm). All the modified nucleases were found to strongly bind to the cell membrane and begin internalization (FIGS. 21A-21D).

Validation of Delivery of the Modified Nucleases into Jurkat T-Cells and Primary T-Cells

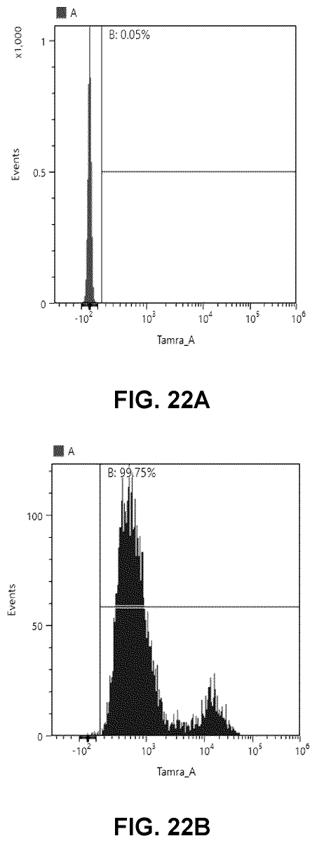

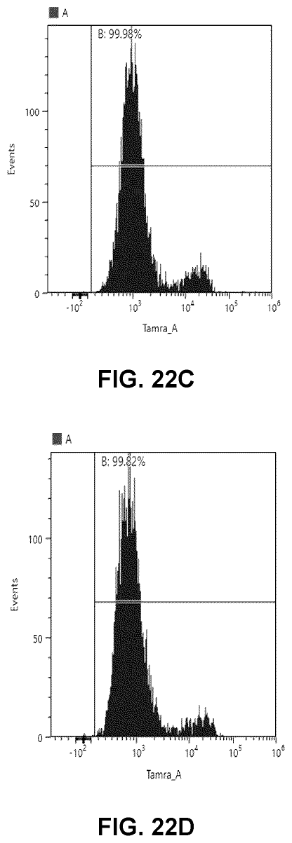

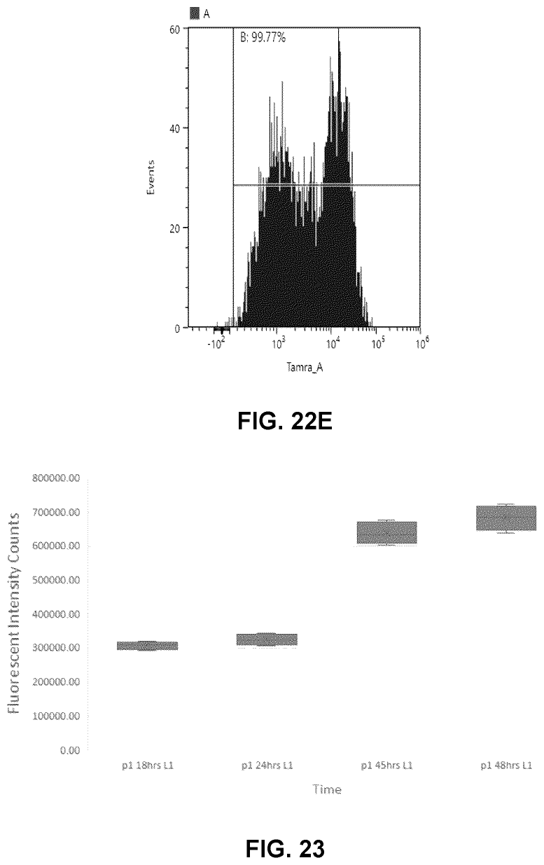

Jurkat CD4+ T cell leukemia cells were maintained in RMP1™ 1640 media with the addition of 10% fetal bovine serum (FBS). 25′000 cells were seeded in wells of a 96 well plate. Each well was treated with 10 micrograms of each protein (Zero, L1, L2, or L3) that was labeled with a TMRA fluorophore and incubated for 1 hour. The cells were then harvested, washed and fixed with 5% formalin for 1 h before being spun down and re-suspended in PBS+1% FBS. The cells were stored at 4° C. until analyzed using a Sony™ Cell spectroanalyzer 107000 flow cytometer. Fluorescence was detected in the TAMRA fluorescence range with autofluorescence removal (TAMRA excitation 555 nm, emission 580 nm) and a whole fluorescent spectrum was acquired. The control used cells cultured under the same conditions but without adding a nuclease. All proteins achieved cell binding with L3 producing the most significant fluorescence signal reflecting higher degree of cell binding and accumulation (FIGS. 22A-22E). Over 99% of delivery was achieved for each of Zero, L1, L2, and L3 which indicates the efficient internalization of the modified nucleases described herein.

Monitoring of GFP was performed by an in vivo imaging system (IVIS) as a time course assay using the Jurkat T-cells treated with L1 protein construct (for GFP genome integration) were evaluated over the course of 48 hours. GFP signal consistently increased over the duration for the experiment, indicating that CAR donor has been integrated by HDR (FIG. 23). To evaluate the degree of integration across the cell population all constructs were evaluated by flow cytometry. Human Primary CD4+ T-cells (seed 20′000 cells per well, 96 well plate) were maintained in RMP1™ 1640+10% FBS and treated with 10 micrograms of each protein, GFP signal was monitor at the 18 hours, 24 hours, 45 hours and 48 hours stages tracking performance of L1 constructs. With successful observation of increasing GFP signal (FIG. 23) it was inferred due to CAR template construct that successful HDR had resulted. GFP can only be expressed if the whole CAR construct has been inserted to the genome as the CAR construct lacks a promoter and is driven off the endogenous promoter at the TRAC locus. As can be seen in FIG. 23, the GFP signal nearly doubled from 18 hours to 48 hours which is indicative of gene editing resulting in GFP expression.

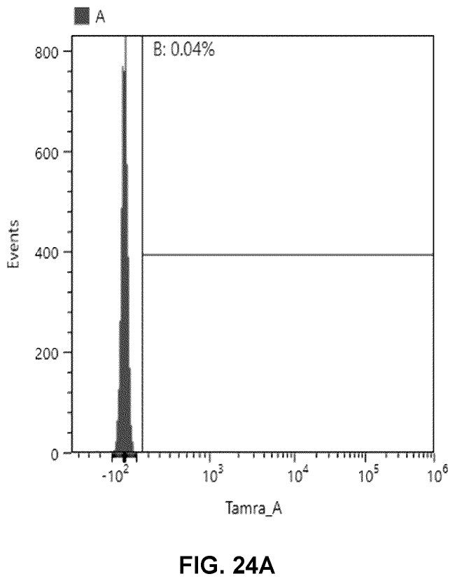

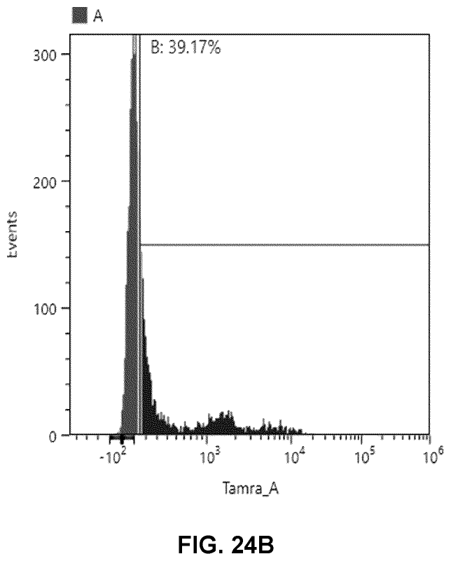

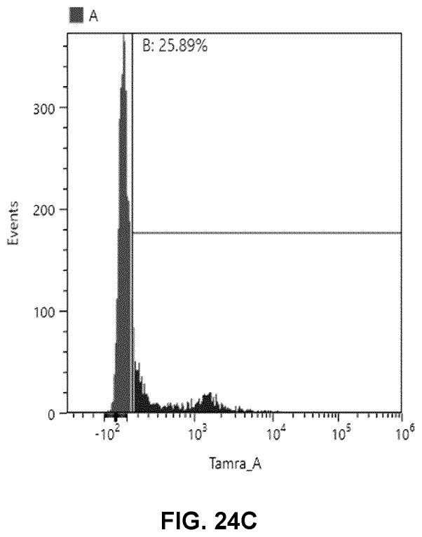

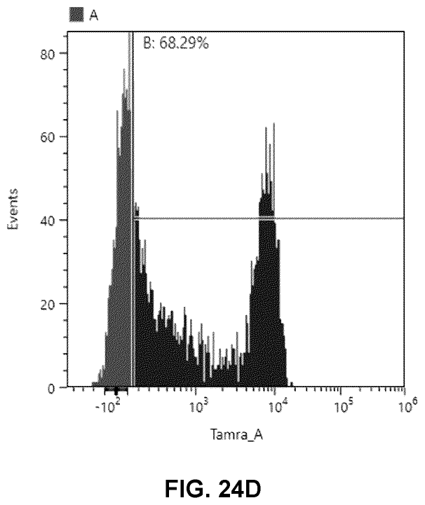

The validation delivery experiment was repeated in a clinically relevant model: primary T-cells. 20′000 cells of Human Primary CD4+ T-cells were seeded wells of a 96 well plate. The cells were maintained in RMP1™ 1640+10% FBS and treated with 10 micrograms of each protein (Zero, L1, L2, or L3) that was labeled with a TMRA fluorophore and incubated for 1 hour. The cells were then harvested, washed and fixed with 5% formalin for 1 h before being spun down and re-suspended in PBS+1% FBS. The cells were stored at 4° C. until analyzed using a Sony™ Cell spectroanalyzer ID7000 flow cytometer. Fluorescence was detected in the TAMRA fluorescence range with autofluorescence removal (TAMRA excitation 555 nm, emission 580 nm) and a whole fluorescent spectrum was acquired. The control used cells cultured under the same conditions but without adding a nuclease. All proteins (Zero, L1, L2, or L3) achieved cell binding with L3 producing the most significant fluorescence signal reflecting a higher degree of cell binding and accumulation (FIGS. 24A-24D).

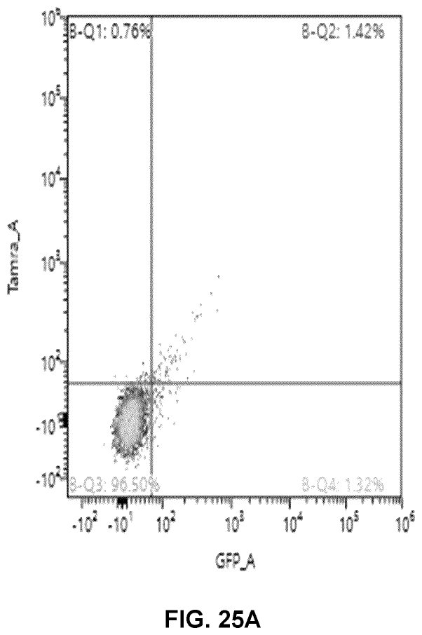

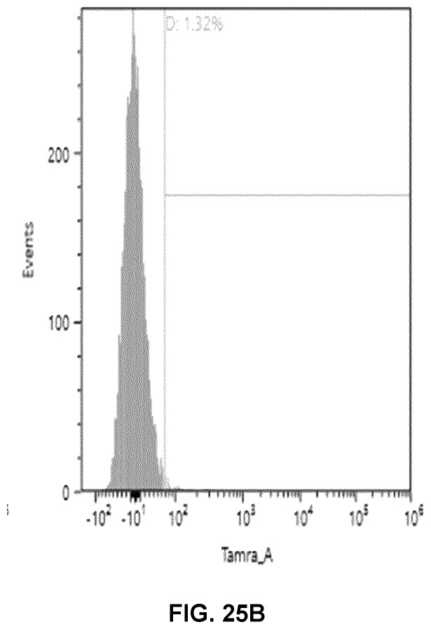

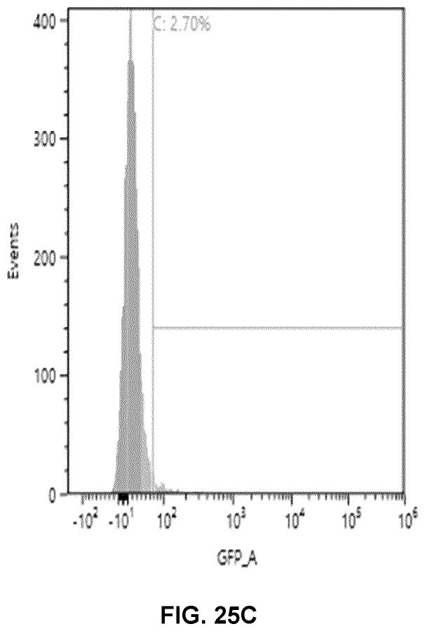

Using spectral flow cytometry, delivery and CAR integration was evaluated in the TAMRA and GFP channel respectively. Efficient delivery to Jurkat cells (>90%) was evaluated at various nuclease protein concentrations (0.33, 16, 33.3 and 66 ng/μL) of modified nucleases (Zero, L1, L2, and L3) and for incubations times of 48 h and 72 h. It was observed that as the nuclease protein concentration increases the efficiency of CAR-GFP donor integration increases with a commensurate increase in GFP expressing cells in all protein constructs after 48 h and 72 h of incubation. (FIGS. 25A-25C, FIGS. 26A-26L, FIGS. 27A-27L, FIGS. 28A-28L, FIGS. 29A-29L, FIGS. 30A-30C, FIGS. 31A-31L, FIGS. 32A-32L, FIGS. 33A-33L, FIGS. 34A-34L, and Table 18 below). With TAMRA detection in excess of 104 log intensity, the chance of high efficiency editing increases. Over 72 hours the GFP increased in the wells by two mechanisms: cell expansion and HDR editing inserting the CAR template. When considering the flow results the GFP representative fraction continued to increase indicating that editing persists through 72 hours, though the overall rate of increase observed at 48 hours was less. Cell viability at the completion of the experiment remained good at over 90% for all conditions, cell numbers had expanded from an initial seed of 25′000 to around 75-80′000 cells.

TABLE 18
GFP representative fraction (in percentage)
determined by flow cytometry

| | | |
|---|---|---|
| Incubation | | |
| time (h) | Concentration | GFP % detected |

| | | | |
|---|---|---|---|
| 48 | 0.33 | ng/μL of Zero | 2.43 |
| 48 | 16 | ng/μL of Zero | 7.04 |
| 48 | 33.3 | ng/μL of Zero | 12.66 |
| 48 | 66 | ng/μL of Zero | 20.38 |
| 48 | 0.33 | ng/μL of L1 | 5.58 |
| 48 | 16 | ng/μL of L1 | 11.9 |
| 48 | 33.3 | ng/μL of L1 | 29.94 |
| 48 | 66 | ng/μL of L1 | 87.72 |
| 48 | 0.33 | ng/μL of L2 | 7.1 |
| 48 | 16 | ng/μL of L2 | 14.36 |
| 48 | 33.3 | ng/μL of L2 | 86.2 |
| 48 | 66 | ng/μL of L2 | 89.9 |
| 48 | 0.33 | ng/μL of L3 | 16.36 |
| 48 | 16 | ng/μL of L3 | 20.32 |
| 48 | 33.3 | ng/μL of L3 | 21.5 |
| 48 | 66 | ng/μL of L3 | 34.3 |
| 72 | 0.33 | ng/μL of Zero | 10.16 |
| 72 | 16 | ng/μL of Zero | 16.28 |
| 72 | 33.3 | ng/μL of Zero | 25.1 |
| 72 | 66 | ng/μL of Zero | 17.06 |
| 72 | 0.33 | ng/μL of L1 | 9.98 |
| 72 | 16 | ng/μL of L1 | 26.28 |
| 72 | 33.3 | ng/μL of L1 | 89.98 |
| 72 | 66 | ng/μL of L1 | 85.6 |
| 72 | 0.33 | ng/μL of L2 | 7 |
| 72 | 16 | ng/μL of L2 | 12.78 |
| 72 | 33.3 | ng/μL of L2 | 92.5 |
| 72 | 66 | ng/μL of L2 | 94.74 |
| 72 | 0.33 | ng/μL of L3 | 10.68 |
| 72 | 16 | ng/μL of L3 | 17.22 |
| 72 | 33.3 | ng/μL of L3 | 51.12 |
| 72 | 66 | ng/μL of L3 | 71.88 |

The most success was found with L1 and L2 which achieved a GFP signal in 87 and 90% at the 48 hr stage. Further evaluation at the 72 hr stage revealed further improvement. Editing efficiency was measured using GFP reporter encoded downstream of the CAR receptor insert increase with increasing modified nucleases concentration. It was found that Zero was a mitigated nuclease in that its enzymatic capacity has been reduced by the positioning of the delivery domain, impacting its ability to form double strand breaks. The activity of Zero improved with increasing concentrations. The presence of a linker domain in L1 and L2 provided a favorable protein orientation, position and folding. In L3, the overall protein folding and shape was not significantly changed since a small CDR was inserted at the SP3 site in the loop. The performance of L3 was better than Zero. Overall a high frequency of genetic delivery was demonstrated which results in a high efficiency genetic editing. The percent GFP reported in the Table 18 above is based on the linear gating of the histograms rather than the quadratic division. The quadratic division with TAMRA and GFP as Y and X axis was provided as a means to evaluate the impact of high degrees of modified nuclease delivery and its correlation with high degrees of HDR resulting in GFP signal (FIGS. 25A, 26A, 260, 26G, 26J, 27A, 270, 27G, 27J, 28A, 280, 28G, 28J, 29A, 29D, 29G, 29J, 30A, 31A, 31D, 31G, 31J, 32A, 32D, 32G, 32J, 33A, 33D, 33G, 33J, 34A, 34D, 34G, and 34J).

Using primers (Table 17) that bridge the C-terminus of the CD3z of the CAR receptor to the GFP sequence after the P2A site amplification, the confirmation of insertion was achieved by PCR. Genomic DNA was extracted from Jurkat cells that were treated with 66 ng/μL at the 72 hrs stage. Using the insert specific primers, a 600 bp fragment of the insert was amplified from genomic DNA extracted from control (Jurkat untreated), Z-10, L1-10, L2-10 and L3-10, where 10 denotes 66 ng/μL concentration. Lack of insert amplification would exclude CAR insertion, whereas the presence of a PCR product would confirm insertion. For Zero, L1, L2 and L3 the product (˜700 bp) was confirmed by agarose gel (1.5%, TAE buffer) whereas control Jurkat cells exhibited no insert (FIG. 35).

The spectral flow cytometry analysis performed on Jurkat T cells was repeated on primary T cells using no nuclease as control and 66 ng/μL of L1, L2, or L3 with a donor CAR and TRAC sgRNA. At the 24 hour mark the GFP signal was detected and was indicative of cell proliferation. At the 48 hour mark 99% of the primary T cells were successfully delivered L1 or L2 and the gene editing achieved was over 63-71.6% (FIGS. 36A-36L).

### CD4 T Cell Delivery In Vivo

Injection of NOD scid gamma (NSG) mice was performed at 1/12 maximum tolerated dose (MTD) (namely 150 μg) of L1 protein or L2 protein complex in molar ratio 1:1:1 of protein:sgRNA:biotinylated donor CAR, in 150 μL of PBS as carrier. L1 and L2 proteins were already labeled with TAMRA NHS reagent and were purified by gel filtration. Mice to be injected were obtained from JAX laboratories. In brief, their engraftment was achieved in the following manner: female mice were injected with human hematopoietic stem cells (hu-CD34⁺), with mature CD45⁺ cells confirmed prior to delivery. A single human donor was used. Injection was performed via tail vein and first blood sample was taken at 3 h post injection to evaluate firstly CD4+ T-cells and secondly if delivery had occurred. L1 was selected to evaluate early delivery. NSG (NOD.Cg-Prkdcscid II2rgtm1Wjl) CD34+ human stem cell grafter mice were pre-treated with interleukin 7 (IL-7) prior to injection of L1 or L2 at time minus 4 days and time minus 1 day and one day post injection. The purpose was to boost T-cell numbers post arrival of NSG mice and equalise the variability of engraftment. At 3 h, 24 h, and 48 h post injection, 20 microliters of whole blood was taken from the tail in a heparin capillary to prevent coagulation. Capillary was spun at 14 k rotations per minute (RPM) for 4 minutes to separate serum and whole blood. Red blood cells (RBC) were lysed with 400 μL of RBC lysis buffer (1×) for 20 minutes at room temperature, before centrifugation at 3500 RPM for 4.5 minutes was performed to separate white blood cells and supernatant was discarded. White blood cells were resuspended in PBS+1% FBS, and stained with 5 μg of APC antiCD4 antibody (Biolegend™) for 15 minutes, followed by centrifugation and wash with PBS+1% FBS before resuspension and analysis by flow cytometry. A control sample was derived from untreated NSG mouse and L1 samples from a mouse treated with L1. Flow cytometry analysis was performed with dual gating for APC and TAMRA on a Sony™ Spectral Analyzer flow cytometer with a minimum of 5000 cell analysed, with autofluorescent subtraction. The existence of a CD4+ and Tamra positive cell population at 3 h for L1 validated that targeting of CD4+ T cells in-vivo was achieved (FIGS. 37A and 37B). Furthermore, at 3 h L2 showed that it also achieved CD4+ T-cell delivery, reaching 24.5% delivery (FIGS. 37A and 37C). At 3 h, the NSG mice treated with L1 and independently with L2 achieved 13.22% and 24.5% respectively of the population analyzed of showed positive TAMRA signal, correlated to delivery of the L1 or L2 Tamra labelled protein into that cell population. It is noted that at 1/12 MTD there is significant headroom for improvement of delivery to the total T-cell population. At 24 h and 48 h (FIGS. 37D-371), a drop was observed in overall intensity of TAMRA, as L1 or L2 cleared from the serum. While L1 maintained a 15% positive signal the overall intensity dropped to 10²⁵. To evaluate the clearance of L1 and L2 from the cells, the TAMRA signal was considered cleared when it dropped below that of the control (i.e. 2%).

With clearance of L1 or L2 TAMRA from the CD4+ T cells at 48 h, the spectral analysis channel was opened for PE-CD19 (phycoerythrin or PE, a fluorophore tagging CD19) staining to evaluate CAR expression. Following the demonstration that L1 and L2 are cleared at 48 h, it is therefore appropriate to evaluate the internalization and gene editing of L1 and L2 through the expression of PE-CD19 at 48 h. The sample protocol for blood harvesting, cell preparation and washing was followed but with PE-CD19 added at the same time as APC-antiCD4 at the same concentration. Incubation was 15 minutes at room temperature with washing prior to analysis. PE-CD19 filter was used for analysis on the Sony™ spectral analyzer flow cytometer and the results presented represent the 48 samples from representatively L1 and L2 treated animals plus untreated control (FIGS. 38A-38C). With an increase in CD19 staining for both L1 and L2 treated animals it was demonstrated that CD19 expressing CAR-T-cells can be produced in vivo. At this stage, the challenge was introduced to the animals to encourage the action and expansion of in vivo generated anti-CD19 CAR CD4+ T-Cells. This was provided by a 1×10⁶ tail vein injection of Raji-Luc CD19+ (B cell lymphocytes of burkitt lymphoma origin adapted with a luciferase expressing cassette to enable bioluminescence evaluation of viability in-vivo. Bioluminscence was evaluated by 10 μg/mL injection intraperitoneally of luciferin to each animal with imaging occurring 7 mins post injection (at 0.5 second exposure fstop1, binning factor 8) using an IVIS whole animal imager. Post injection, the mice were evaluated on a bi-weekly basis, shaved upon the torso once a week to enable accurate quantification of bioluminescence by the limitation of scattering and absorbance caused by hair. Engraftment and expansion of the xenograft was validated at 4 day post injection and by the 7^{th} day it became apparent that bioluminescence was diminished in the L1 and L2 treated animals in comparison to the control vehicle (untreated PBS injection) animals.

At 192 hours post injection of Raji cells, it was observed that growth and cell viability of Raji cells was retarded compared to untreated control Raji in NSG mice (FIGS. 39A and 39B). Biological replicates were n=4 for control (PBS vehicle) (labeled as C), n=3 for L1 (labeled as L1) and n=4 for L2 (labeled as L2). The injection was administered at 1×10⁶ cells in 150 μL. The expression of anti CD19 chimeric antigen receptor was observed by flow cytometry. The bioluminescence intensity was measured by interperiternal injection of luciferin, Raji cells expressing luciferase convert this to a bioluminescent signal, measuring cell viability and proliferation. It was observed that animals treated with L1 or L2 and expressing CD19 positive CD4+ T cells retarded the growth of CD19 expressing Raji cell in vivo. The signal was detected by IVIS luminescence imaging using the following conditions: 0.5 second, Fstop: 1, binning 8 and 7 minutes post injection of 100 μL (10 μg/mL) I.P.

Dfd for streptavidin aptamer modifications of donor template for insertion of CAR. Importance is donor no longer requires a biotinylation modification and the aptamer can be used to binding to MAV domain of 6.0 nuclease nabs. Sequences of primers used to amplify donor with addition of streptavidin aptamer are provided in addition of the figure

Initial CAR donor synthesis involved a PCR amplification of a linear template with the Forward primer adding a biotin to the 5′ end of donor. While this can be scaled to manufacturing two simpler strategies can be applied:
Amplification of donor upon a plasmid followed by plasmid purification, restriction digestion, purification and enzymatic addition of biotin, final purification, or
Amplification of donor with an avidin binding sequence at 5′ end upon a plasmid followed by plasmid preparation, restriction digest and final gel filtration. The addition of a DNA encoded avidin binding removes the need for additional purification and enzymatic treatment. Effectively a completely functional donor can be encoded directly in the DNA.

TABLE 19
Streptavidin aptamers

| | | SEQ |
|---|---|---|
| | | ID |
| Aptamer | | NO |
| St-2-t- | TTGACCGCTGTGTGACGCAACACTC | 354 |
| 2 | AAGCCTGTCCCTGAGTCCCA | |
| ST-2-1 | ATTGACCGCTGTGTGACGCAACACT | 355 |
| | CAATGCCTGTCCCTGAGTCCCA | |
| 17- | ATCTCCGATTGCCCCACGACGCAGT | 356 |
| f1OLD | GGTCGGAGTTACTTTGCCTGTCCCT | |
| | GAGTCCCA | |

Gel retardation assay (FIG. 40A) shows 4 double stranded linear DNA donors of 2.4 kb length. 1 donor has a Biotin and the others Strep-Apt DNA aptamers that bind to avidin (Table 19). Donors migrate relative to their size in the gel. When complexed with protein avidin binding domain either through biotin or strep-Apt aptamers in donors were retarded in the wells and prevented from migrating down the gel. A 1:1 molar ratio DNA to PNME is maintained for both standard donor and the 3 strep-apt donors variants (Table 19).

An off target analysis for L2 treated cells was also performed. The the top ten most likely sites were selected and amplified using PCR. If insertion has occurred with high frequency, then a larger increase in amplicon length will be observed as donor is 2.4 kb long. Amplicons are centred upon the predicted off target cut sites and are 750-800 bp in length when no insert is present. If insert is present length should increase substantially. The samples selected were from ex-vivo cell experiments with jurkat control samples (untreated) and L2 treated at highest concentration (66 ng/μL) at the 48 hrs stage. DNA was extracted from each sample and PCR performed using locus specific primers. FIG. 40B shows chromosome where off targets are located. Amplicons for each sample are presented side by side. Amplicons of consistent size and absence of multiple bands between control and L2 treated cells was observed which suggests strongly that high frequency insertions are unlikely to have occurred (˜750-800 bp). Sanger confirmation of sequence confirmed no insertion at cut sites. Indel analysis showed no indels at below the 1.5% certainty threshold (tracking o indels by decomposition (TIDE)). Equivalent of Control and L2 samples regarding indels and insertions is indicated by “Y” below each sample on gel.

1. A composition for modifying a T cell, the composition comprising:
a protein complex comprising a polynucleotide-modifying enzyme domain, a T cell membrane binding domain and an endosome escape domain;
a guide oligonucleotide specific to a T cell receptor a constant (TRAC) gene of the T cell; and
a donor DNA comprising two homology arms at each end of the donor DNA homologous to exon1 of the TRAC gene and encoding therebetween a chimeric antigen T cell receptor comprising:
a translocation signal for translocation to a cell membrane of the T cell;
a transmembrane domain;
an intracellular signaling domain; and
an extracellular antigen binding domain.

2. The composition of claim 1, wherein the protein complex further comprises a hapten binding domain.

3. The composition of claim 2, wherein the donor DNA is conjugated to a hapten and the hapten binds the hapten binding domain.

4. The composition of claim 1, wherein the protein complex further comprises a nuclear localisation sequence.

5. The composition of claim 1, wherein the chimeric antigen T cell receptor further comprises a CD8 hinge region.

6. The composition of claim 1, wherein the chimeric antigen T cell receptor further comprises a B cell lymphoma recognition domain.

7. The composition of claim 1, wherein the guide oligonucleotide is complementary to a sequence located between 250 nucleotides before the start codon of the exon 1 of the TRAC gene to 250 nucleotides after the start codon of the exon 1 of the TRAC gene.

8. The composition of claim 1, wherein the polynucleotide-modifying enzyme domain is covalently linked to the endosome escape domain.

9. The composition of claim 1, wherein the T cell membrane binding domain is a cationic peptide or a cell recognition domain that targets CD4, CD8, CD16 or CD56, and wherein
i) the cell recognition domain is covalently coupled to the endosome escape domain,
ii) the cell recognition domain is a display domain being a peptidic recognition sequence of from 3 to 20 amino acids in length positioned in a loop or alpha helix on an external surface of the polynucleotide-modifying enzyme domain, and wherein the peptidic recognition sequence is a complementarity-determining region (CDR), or
iii) the cell recognition domain is an antigen binding domain selected from Fab, single-domain antibody (sdAb), V_{HH}, or camelid antibody domain, positioned in a loop on an external surface of the polynucleotide-modifying enzyme.

10.-15. (canceled)

16. The composition of claim 1, wherein the polynucleotide-modifying domain is a type II Cas, a functional analog thereof, a variant thereof or a derivative thereof.

17. The composition of claim 16, wherein the type II Cas is Cas9, a functional analog thereof, a variant thereof or a derivative thereof.

18. The composition of claim 1, the polynucleotide-modifying domain is a type V Cas, a functional analog thereof, a variant thereof or a derivative thereof.

19. The composition of claim 1, wherein the extracellular antigen binding domain is specific to a cancer specific antigen.

20.-23. (canceled)

24. A method of performing cellular therapy for a subject in need thereof, the method comprising providing ex vivo allogenic T cells, modifying the genome of the T cells with the composition as defined in claim 1 to obtain chimeric antigen receptor (CAR) T cells, and administering the CAR T cells to the subject.

25. (canceled)

26. A method of treating cancer for a subject in need thereof, the method comprising providing allogenic T cells, modifying the genome of the T cells with the composition as defined in claim 19 to obtain CAR T cells, and administering the CAR T cells to the subject.

27.-29. (canceled)

30. A polynucleotide-modifying enzyme comprising:
a functional nuclease domain comprising a nuclease catalytic pocket;
an antigen binding domain selected from Fab, single-domain antibody (sdAb), V_{HH}, or camelid antibody domain, in a loop that is positioned on an external surface of the polynucleotide-modifying enzyme, and said antigen binding domain recognizes a target cell receptor of a target cell to allow cell internalization of the polynucleotide-modifying enzyme in said target cell; and
a linker of from 0 to 30 amino acids, upstream of the antigen binding domain.

31. The polynucleotide-modifying enzyme of claim 30, wherein the nanobody is a VHH.

32. The polynucleotide-modifying enzyme of claim 30, wherein the linker sequence is from 16 to 23 amino acids.

33. The polynucleotide-modifying enzyme of claim 30, wherein the nuclease catalytic pocket is a Cas nuclease catalytic pocket, recombinase catalytic pocket or a meganuclease catalytic pocket, and wherein the Cas is:
b) Cas9, a functional analog thereof, a variant thereof or a derivative thereof and wherein the nuclease catalytic pocket comprises a HNH nuclease domain,
c) Cas12, a functional analog thereof, a variant thereof or a derivative thereof,
d) Cas13, a functional analog thereof, a variant thereof or a derivative thereof, or
e) Cas14, a functional analog thereof, a variant thereof or a derivative thereof.

34.-41. (canceled)

42. The polynucleotide-modifying enzyme of claim 30, wherein the nuclease catalytic pocket comprises a RuvC nuclease domain.

43. (canceled)


SEQUENCE LISTING (356 sequences)

SEQ ID NO: 1 - DNA, 4104 bp, synthetic construct
  source 1..4104: mol_type = other DNA
atggataaaaaatacagcattggtctggacattggcacgaatagcgttggttgggcagtgattaccgatgaatacaaagtcccgtcgaaaaaattcaaagtgctgggtaacaccgatcgccatagcattaagaaaaacctgatcggtgcgctgctgtttgattctggcgaaaccgcggaagcaacgcgtctgaaacgtaccgcacgtcgccgttacacgcgccgtaaaaatcgtatttgctatctgcaggaaatctttagcaacgaaatggcgaaagtcgatgactcatttttccaccgcctggaagaatcgtttctggtggaagaagataaaaaacatgaacgtcacccgattttcggcaatatcgttgatgaagtcgcgtaccatgaaaaatatccgacgatttaccacctgcgtaaaaaactggtggattctaccgacaaagccgatctgcgcctgatttatctggcactggctcatatgatcaaatttcgtggtcacttcctgattgaaggcgacctgaacccggataatagtgacgtcgataaactgtttattcagctggtgcaaacctataatcagctgttcgaagaaaacccgatcaatgcaagtggtgttgatgcgaaagccattctgtccgctcgcctgagtaaatcccgccgtctggaaaacctgattgcacagctgccgggtgaaaagaaaaacggtctgtttggcaatctgatcgctctgtcactgggcctgacgccgaactttaaatcgaatttcgacctggcagaagatgctaaactgcagctgagcaaagatacctacgatgacgatctggacaacctgctggcgcaaattggcgaccagtatgccgacctgtttctggcggccaaaaatctgtcagatgccattctgctgtcggacatcctgcgcgtgaacaccgaaatcacgaaagcgccgctgtcagcctcgatgattaaacgctacgatgaacatcaccaggacctgaccctgctgaaagcactggttcgtcagcaactgccggaaaaatacaaagaaattttctttgaccaaagtaaaaatggttatgcaggctacatcgatggcggtgcttcccaggaagaattctacaaattcatcaaaccgatcctggaaaaaatggatggtacggaagaactgctggtgaaactgaatcgtgaagatctgctgcgtaaacaacgcacctttgacaacggtagcattccgcatcagatccacctgggcgaactgcatgcgattctgcgccgtcaggaagatttttatccgttcctgaaagacaaccgtgaaaaaatcgaaaaaatcctgacgtttcgcatcccgtattacgttggtccgctggcacgtggtaatagccgcttcgcatggatgacccgcaaatctgaagaaaccattacgccgtggaactttgaagaagtggttgataaaggcgcaagcgctcagtcttttatcgaacgtatgaccaatttcgataaaaacctgccgaatgaaaaagtgctgccgaaacattctctgctgtatgaatactttaccgtttacaacgaactgacgaaagtgaaatatgttaccgagggtatgcgcaaaccggcgtttctgagtggcgaacagaaaaaagccattgtggatctgctgttcaaaaccaatcgtaaagttacggtcaaacagctgaaagaagattacttcaagaaaattgaatgtttcgacagcgtggaaatttctggtgttgaagatcgtttcaacgcctctctgggcacctatcatgacctgctgaaaatcatcaaagacaaagattttctggataacgaagaaaacgaagacattctggaagatatcgtgctgaccctgacgctgttcgaagatcgtgaaatgattgaagaacgcctgaaaacgtacgcacacctgtttgacgataaagttatgaaacagctgaaacgccgtcgctataccggttggggccgtctgagccgcaaactgattaatggtatccgcgataaacaatcaggcaaaacgattctggatttcctgaaatcggacggctttgccaaccgtaatttcatgcagctgatccatgacgattccctgacctttaaagaagacattcagaaagcacaagtgtcaggtcaaggcgattcgctgcatgaacacattgcgaacctggccggttcaccggctatcaaaaaaggcatcctgcagaccgtgaaagtcgtggatgaactggtgaaagttatgggtcgtcacaaaccggaaaacattgttatcgaaatggcgcgcgaaaatcagaccacgcaaaaaggccagaaaaactcgcgtgaacgcatgaaacgcattgaagaaggtatcaaagaactgggcagccagattctgaaagaacatccggtcgaaaacacccagctgcaaaatgaaaaactgtacctgtattacctgcaaaatggtcgtgacatgtatgtggatcaggaactggacatcaaccgcctgtctgactatgatgtcgaccacattgtgccgcagagctttctgaaagacgattctatcgataacaaagttctgacccgtagtgataaaaaccgcggcaaaagcgacaatgtcccgtctgaagaagttgtgaagaaaatgaaaaactactggcgtcaactgctgaatgcgaaactgattacgcagcgtaaattcgataacctgaccaaagcggaacgcggcggtctgtccgaactggataaagccggttttatcaaacgtcaactggttgaaacccgccagattacgaaacatgtcgcccagatcctggattcacgcatgaacacgaaatacgacgaaaacgataaactgatccgtgaagtcaaagtgatcaccctgaaaagtaaactggtttccgatttccgtaaagactttcagttctacaaagtccgcgaaattaacaattaccatcacgcacacgatgcttatctgaatgcagtggttggtaccgctctgatcaaaaaatatccgaaactggaaagcgaatttgtgtatggcgattacaaagtctatgacgtgcgcaaaatgattgcgaaatccgaacaggaaatcggcaaagcgaccgccaaatactttttctattcaaacatcatgaactttttcaaaaccgaaattacgctggcaaatggtgaaattcgtaaacgcccgctgatcgaaaccaacggtgaaacgggcgaaattgtgtgggataaaggccgtgacttcgcgaccgttcgcaaagtcctgtcgatgccgcaagtgaatatcgtgaagaaaaccgaagtgcagacgggcggttttagtaaagaatccatcctgccgaaacgtaacagcgataaactgattgcgcgcaaaaaagattgggacccgaaaaaatacggcggttttgatagtccgacggttgcatattccgtcctggtcgtggctaaagtcgaaaaaggtaaaagtaaaaaactgaaatccgtgaaagaactgctgggcattaccatcatggaacgtagctcttttgagaaaaacccgattgacttcctggaagccaaaggttacaaagaagtgaaaaaagatctgatcatcaaactgccgaaatatagcctgttcgaactggaaaacggccgtaaacgcatgctggcatctgctggtgaactgcagaaaggcaatgaactggcactgccgagtaaatatgttaactttctgtacctggctagccattatgaaaaactgaaaggttctccggaagataacgaacagaaacaactgttcgtcgaacaacataaacactacctggatgaaatcatcgaacagatctcagaattctcgaaacgcgtgattctggcggatgccaatctggacaaagttctgagcgcgtataacaaacatcgtgataaaccgattcgcgaacaggccgaaaatattatccacctgtttaccctgacgaacctgggcgcaccggcagcttttaaatacttcgataccacgatcgaccgtaaacgctatacctcaacgaaagaagttctggatgctaccctgattcatcaatcgatcaccggtctgtatgaaacgcgtattgatctgagtcagctgggcggtgac

SEQ ID NO: 2 - protein, 1367 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGG

SEQ ID NO: 3 - DNA, 3684 bp, synthetic construct
  source 1..3684: mol_type = other DNA
atgtcaaagctggagaaattcaccaactgttatagcctgtctaagaccctgcgcttcaaggcaatcccagtgggcaagacacaagagaacattgacaacaaacggctcctggtggaggatgagaagagggctgaagattacaagggcgttaagaagctgctggataggtactatctgtcattcatcaacgatgtcctccacagtatcaagctgaagaatctgaacaattacatttctctgttccggaagaagacacggaccgagaaggagaacaaagagctggagaatctggagatcaacctgaggaaagaaatagctaaggctttcaaagggaacgagggttacaagtccctgttcaagaaagacattatcgagactattctgcctgagttcctggacgataaagatgagatcgccctcgtcaattccttcaatgggtttaccacagcctttaccggcttcttcgacaatagagagaatatgttctctgaagaggccaaatccactagcatcgcctttcgctgcataaacgagaacctgactaggtacatcagcaatatggacatctttgagaaagtcgatgccatattcgacaaacatgaggtgcaggagattaaggagaagatcctgaactcagattacgatgtcgaagatttcttcgagggagagttcttcaacttcgtgctcacacaagagggcattgatgtgtacaatgcaatcattggagggttcgtgacagagagtggcgagaagataaagggcctgaacgagtatatcaacctctacaaccagaaaaccaagcagaaactgcctaagttcaagccactgtacaaacaagtgctctcagatagggaaagcctgagcttctacggtgaagggtatacatcagatgaagaagtgctcgaagtgttccgcaacaccctcaataagaacagtgaaatcttctcttcaatcaagaagctggagaaactgttcaagaatttcgatgagtactcctctgccggaatctttgtgaagaatggccctgcaatatccactattagcaaagacatctttggcgagtggaacgttatcagggataagtggaatgccgagtacgatgatattcatctcaagaagaaagccgtggttacagagaaatacgaggatgatagacgcaagagctttaagaagattggtagcttctctctcgaacagctgcaggagtacgccgacgctgacctgtcagtcgtggagaaactcaaggagatcataatccagaaggtggatgaaatctacaaagtgtatggaagctctgagaaactcttcgatgcagactttgttctggagaagagtctgaagaagaacgacgcagtggttgctatcatgaaggacctgctggattctgttaagtctttcgagaattacattaaggcattctttggtgaagggaaggagacaaatagggacgagagcttctatggcgactttgttctggcctacgacatcctcctcaaggttgaccacatctatgacgctatacggaattacgttacccagaagccctatagcaaagacaagttcaagctgtatttccagaatccacagtttatgggtgggtgggataaagacaaagaaacagattacagggccactatcctgcggtacggcagcaaatactatctggctatcatggataagaagtacgccaaatgcctccagaagatcgacaaggacgacgtgaacggtaactacgagaagatcaattacaagctcctgccaggacctaacaagatgctgcccaaggtgttcttctccaagaaatggatggcctactataacccaagcgaggacattcagaagatatacaagaatgggacattcaagaagggcgatatgttcaacctcaacgactgccacaagctgattgatttcttcaaggatagcatttctcgctatcccaagtggtctaatgcatacgatttcaacttcagcgagactgagaagtacaaagacatcgctggcttctaccgggaggtggaagagcaaggctataaggtgtcattcgaatccgcttctaagaaggaagtggataagctcgtggaagagggtaagctgtacatgttccagatatacaacaaagacttcagcgataagagccacggcactccaaacctccatactatgtatttcaagctgctgtttgacgagaacaaccacggacagattaggctgtcaggaggcgcagaactcttcatgcgcagagcttcactgaagaaggaggaactcgttgtccacccagccaatagccctatagccaataagaatccagacaatcctaagaaaaccactactctgtcttacgatgtgtataaggataagagattctctgaagatcagtacgaactgcacatacccattgccattaacaagtgccctaagaacatcttcaagattaacacagaggttagagtgctcctgaaacacgacgataacccttatgttataggcattgatcgcggagagagaaacctgctgtacatcgtcgtggtggacggcaaaggcaacatcgtggaacagtacagtctcaatgaaatcattaacaatttcaacggaatccgcattaagaccgactaccattctctcctcgacaagaaggagaaagaaaggttcgaagcaagacagaattggacaagtatagagaatatcaaagaactgaaggctgggtacatctctcaggttgtgcacaagatatgtgagctggtggagaagtacgacgctgttatcgccctcgaggacctgaatagcggcttcaagaactccagggtgaaggtggagaagcaggtgtatcagaagttcgagaagatgctgatcgacaagctcaactatatggtggacaagaaatccaatccttgcgctactggtggagccctgaagggctatcaaatcaccaataagttcgaatctttcaagtctatgagcacccagaatggcttcatcttctacatacccgcatggctgacatccaagattgatccctctaccggatttgttaatctgctcaagactaagtacacctctattgctgactcaaagaagttcatatcatcatttgaccgcatcatgtacgtgccagaagaggacctgttcgagtttgccctggattacaagaatttctctcggactgacgccgactacatcaagaagtggaagctctactcttatggtaatcggattcgcatattccgcaatcccaagaagaataacgtgttcgattgggaggaagtttgcctcaccagcgcttacaaggagctgttcaataagtatgggattaactaccagcagggcgacataagagccctgctgtgcgaacaatctgataaggcattctattcctctttcatggcactgatgtcactgatgctgcaaatgcgcaattccatcaccggaagaacagacgtggactttctgatctctcctgtcaagaactcagatggcatcttctacgattcccgcaactatgaagcacaggagaatgctatcctgcctaagaatgccgatgcaaatggagcctataacatcgccagaaaggtcctctgggccataggacaattcaagaaagctgaagatgagaagctggacaaggtgaagatcgccatttcaaacaaagagtggctcgaatatgctcagacctcagtgaagcat

SEQ ID NO: 4 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 5 - DNA, 3792 bp, synthetic construct
  source 1..3792: mol_type = other DNA
atgaacaacggcacaaataattttcagaacttcatcgggatctcaagtttgcagaaaacgctgcgcaatgctctgatccccacggaaaccacgcaacagttcatcgtcaagaacggaataattaaagaagatgagttacgtggcgagaaccgccagattctgaaagatatcatggatgactactaccgcggattcatctctgagactctgagttctattgatgacatagattggactagcctgttcgaaaaaatggaaattcagctgaaaaatggtgataataaagataccttaattaaggaacagacagagtatcggaaagcaatccataaaaaatttgcgaacgacgatcggtttaagaacatgtttagcgccaaactgattagtgacatattacctgaatttgtcatccacaacaataattattcggcatcagagaaagaggaaaaaacccaggtgataaaattgttttcgcgctttgcgactagctttaaagattacttcaagaaccgtgcaaattgcttttcagcggacgatatttcatcaagcagctgccatcgcatcgtcaacgacaatgcagagatattcttttcaaatgcgctggtctaccgccggatcgtaaaatcgctgagcaatgacgatatcaacaaaatttcgggcgatatgaaagattcattaaaagaaatgagtctggaagaaatatattcttacgagaagtatggggaatttattacccaggaaggcattagcttctataatgatatctgtgggaaagtgaattcttttatgaacctgtattgtcagaaaaataaagaaaacaaaaatttatacaaacttcagaaacttcacaaacagattctatgcattgcggacactagctatgaggtcccgtataaatttgaaagtgacgaggaagtgtaccaatcagttaacggcttccttgataacattagcagcaaacatatagtcgaaagattacgcaaaatcggcgataactataacggctacaacctggataaaatttatatcgtgtccaaattttacgagagcgttagccaaaaaacctaccgcgactgggaaacaattaataccgccctcgaaattcattacaataatatcttgccgggtaacggtaaaagtaaagccgacaaagtaaaaaaagcggttaagaatgatttacagaaatccatcaccgaaataaatgaactagtgtcaaactataagctgtgcagtgacgacaacatcaaagcggagacttatatacatgagattagccatatcttgaataactttgaagcacaggaattgaaatacaatccggaaattcacctagttgaatccgagctcaaagcgagtgagcttaaaaacgtgctggacgtgatcatgaatgcgtttcattggtgttcggtttttatgactgaggaacttgttgataaagacaacaatttttatgcggaactggaggagatttacgatgaaatttatccagtaattagtctgtacaacctggttcgtaactacgttacccagaaaccgtacagcacgaaaaagattaaattgaactttggaataccgacgttagcagacggttggtcaaagtccaaagagtattctaataacgctatcatactgatgcgcgacaatctgtattatctgggcatctttaatgcgaagaataaaccggacaagaagattatcgagggtaatacgtcagaaaataagggtgactacaaaaagatgatttataatttgctcccgggtcccaacaaaatgatcccgaaagttttcttgagcagcaagacgggggtggaaacgtataaaccgagcgcctatatcctagaggggtataaacagaataaacatatcaagtcttcaaaagactttgatatcactttctgtcatgatctgatcgactacttcaaaaactgtattgcaattcatcccgagtggaaaaacttcggttttgattttagcgacaccagtacttatgaagacatttccgggttttatcgtgaggtagagttacaaggttacaagattgattggacatacattagcgaaaaagacattgatctgctgcaggaaaaaggtcaactgtatctgttccagatatataacaaagatttttcgaaaaaatcaaccgggaatgacaaccttcacaccatgtacctgaaaaatcttttctcagaagaaaatcttaaggatatcgtcctgaaacttaacggcgaagcggaaatcttcttcaggaagagcagcataaagaacccaatcattcataaaaaaggctcgattttagtcaaccgtacctacgaagcagaagaaaaagaccagtttggcaacattcaaattgtgcgtaaaaatattccggaaaacatttatcaggagctgtacaaatacttcaacgataaaagcgacaaagagctgtctgatgaagcagccaaactgaagaatgtagtgggacaccacgaggcagcgacgaatatagtcaaggactatcgctacacgtatgataaatacttccttcatatgcctattacgatcaatttcaaagccaataaaacgggttttattaatgataggatcttacagtatatcgctaaagaaaaagacttacatgtgatcggcattgatcggggcgagcgtaacctgatctacgtgtccgtgattgatacttgtggtaatatagttgaacagaaaagctttaacattgtaaacggctacgactatcagataaaactgaaacaacaggagggcgctagacagattgcgcggaaagaatggaaagaaattggtaaaattaaagagatcaaagagggctacctgagcttagtaatccacgagatctctaaaatggtaatcaaatacaatgcaattatagcgatggaggatttgtcttatggttttaaaaaagggcgctttaaggtcgaacggcaagtttaccagaaatttgaaaccatgctcatcaataaactcaactatctggtatttaaagatatttcgattaccgagaatggcggtctcctgaaaggttatcagctgacatacattcctgataaacttaaaaacgtgggtcatcagtgcggctgcattttttatgtgcctgctgcatacacgagcaaaattgatccgaccaccggctttgtgaatatctttaaatttaaagacctgacagtggacgcaaaacgtgaattcattaaaaaatttgactcaattcgttatgacagtgaaaaaaatctgttctgctttacatttgactacaataactttattacgcaaaacacggtcatgagcaaatcatcgtggagtgtgtatacatacggcgtgcgcatcaaacgtcgctttgtgaacggccgcttctcaaacgaaagtgataccattgacataaccaaagatatggagaaaacgttggaaatgacggacattaactggcgcgatggccacgatcttcgtcaagacattatagattatgaaattgttcagcacatattcgaaattttccgtttaacagtgcaaatgcgtaactccttgtctgaactggaggaccgtgattacgatcgtctcatttcacctgtactgaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgcgcgataaactcaaaatcagcaataaagattggttcgactttatccagaataagcgctatctctaa

SEQ ID NO: 6 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 7 - DNA, 3162 bp, synthetic construct
  source 1..3162: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctacagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagttccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtaccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaatcgcctctaagactcagagtatcaaaaagtactcaaccgacattctgggaaacctgtatgaggtgaagagcaaaaagcaccctcagattatcaaaaagggctaa

SEQ ID NO: 8 - protein, 1053 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 9 - DNA, 3921 bp, synthetic construct
  source 1..3921: mol_type = other DNA
atgacccagttcgaggggtttaccaatctgtatcaagtgagcaagacgctgcgctttgaactgatcccacagggaaaaaccttaaaacatattcaagagcagggctttatcgaagaagataaggcccgtaatgaccattacaaagagttaaagccgattattgatcgtatctacaagacctatgcggaccagtgcttacaattggtacagcttgattgggagaacctctctgccgccatcgattcctatcgtaaagaaaaaactgaagaaacgcgcaacgccctgattgaagagcaggccacctatcgtaacgcgattcatgactattttattggccgtacggacaatctgacggacgcgatcaacaagcgccatgcggagatttacaaaggactgtttaaggctgaactgttcaatggtaaggtccttaaacagcttgggaccgtcacaacgacggaacatgaaaacgcgttattacgtagcttcgacaagtttaccacgtatttctccggcttttacgaaaatcgcaaaaacgttttcagtgccgaggatatttccactgctatccctcatcgcattgtgcaagacaacttcccaaaattcaaagaaaattgtcatatcttcacccgcttaatcaccgctgtaccgtccctgcgtgagcatttcgaaaacgtgaaaaaggccattggtatcttcgtgtctacttcgattgaggaggtattttcctttccattctataatcagctgctgacccagacccaaattgatctgtacaaccagctgcttggcggtatttctcgtgaagcaggaaccgaaaaaatcaaagggttgaacgaggtgcttaatctggcaatccagaaaaatgatgaaaccgcccacatcattgcttcgttacctcatcgttttatcccgttgttcaagcaaattttaagtgatcgcaatacgctgtcgtttattctggaagaattcaaaagtgatgaagaggtaattcagtcgttttgcaaatataaaaccctgttacgtaacgaaaatgtcctggaaacagccgaggctttgtttaacgaactgaatagcattgacctgacgcatatctttattagccacaaaaaattagagaccatctcatcagctctgtgcgatcattgggatacactgcgcaatgcgctgtatgaacgtcgtatttcggaattgactggcaaaatcactaaaagcgcgaaagagaaagtacagcgctcgcttaaacatgaagatatcaacctgcaggagatcatcagcgccgcgggtaaagaactgtcggaggcatttaaacagaagacgagcgagattctgtcccacgcacatgccgccttagaccagccgctcccgaccactctgaagaaacaggaagagaaagaaatccttaaaagtcaactggacagtttactgggtctctatcatctgctggattggtttgcggtagacgaaagcaatgaagtggatccggagtttagtgcccgtctgacaggaatcaagctggaaatggagccttcgcttagcttctacaacaaagcccgcaattatgccacgaaaaaaccctatagtgtcgaaaaatttaaactcaactttcaaatgccgacccttgcgtcgggctgggatgtcaacaaagaaaaaaacaacggagctattctgttcgttaaaaatggtctgtactacctgggcatcatgccgaaacagaaaggtcgctacaaagccctttcgttcgagcccacggaaaaaacaagcgaaggcttcgacaaaatgtactacgattactttccggatgcagcaaaaatgatcccgaaatgttccacacagctgaaagccgttacagcacattttcagacgcacaccacccccatcttactgtccaacaattttattgaaccgctggagattactaaagaaatttatgatttgaacaatccggaaaaagagccaaaaaagtttcaaaccgcctacgctaaaaaaaccggggatcagaaagggtaccgcgaagcgttgtgcaagtggattgatttcacccgcgattttctcagtaaatataccaagactacctcgattgacctgagctcactgcgcccgagctctcaatataaggatttgggtgagtactatgctgaattaaaccctttattgtaccacatttcttttcagcgcatcgccgaaaaggaaattatggacgcagtcgaaaccgggaaactgtacctgttccagatctataataaggacttcgccaaaggacatcatggcaaaccgaacctgcacaccctttactggaccgggcttttctctccggaaaatttggcgaaaacctcgatcaagcttaacggtcaagctgagctgttttaccgtccaaaatcccgcatgaagcgcatggcgcatcgtttaggtgaaaaaatgctgaataagaaactgaaagatcagaaaacccctatcccggataccctctaccaggaactgtatgattacgtgaaccatcgtctctcgcatgacctgtcagacgaagcgcgtgcgttactgcccaatgtaatcacaaaagaagtttcgcatgaaattattaaagatcgtcgttttacatctgataaattcttttttcatgttccgatcaccctcaactatcaggccgcaaacagtccaagtaagtttaaccagcgcgttaatgcttacctgaaggaacatccggagactccgattattggaattgatcgcggtgaacgtaatttgatctatatcactgtgatcgatagtaccggtaagattctggagcagcgcagcttgaacacaattcaacagtttgattatcagaaaaaattagacaaccgcgaaaaagagcgcgtggctgcccgtcaggcgtggtctgttgtcggtaccattaaagatctgaagcagggctatctttctcaggttattcacgaaattgtagatctgatgatccattatcaggcggttgttgtgttggagaatctcaatttcggttttaagagtaagcgcacaggcatcgctgaaaaagcagtttatcagcagtttgaaaaaatgctgatcgacaaattgaactgtttagttctcaaagattacccagcggaaaaggtgggcggagtgctgaatccgtaccaattaacggatcaattcacttccttcgcaaagatgggtacccaaagcggctttctgttctatgtgccggccccgtatacctcgaaaatcgatccactgacgggcttcgtagatccgttcgtgtggaaaaccattaaaaatcatgaaagtcgtaaacattttctcgaaggcttcgacttcctgcactacgacgtgaaaactggcgatttcattctgcattttaaaatgaaccgcaacctttcgtttcagcgcggtctgccgggctttatgccggcttgggacattgtttttgagaaaaatgaaacccagtttgatgctaaaggcactcctttcatcgccggtaaacgcatcgtacctgtgattgaaaaccatcgttttacagggcgttaccgtgatttatacccggcgaacgaattgatcgcgctgctggaggaaaagggcatcgttttccgtgacggctccaatattctgccgaaattactggaaaacgacgattcacacgcaattgataccatggtcgcactgattcgctcagtcttacagatgcgtaactctaatgcagccacaggagaagattatattaattcgccagtccgcgatttgaacggtgtttgcttcgacagccgttttcagaatcctgaatggccgatggacgctgatgccaacggagcttatcatatcgccctgaaaggccagctcctgctgaaccacctgaaggaaagcaaagatctgaaattgcagaacggcattagcaaccaggactggttagcatacatccaggaactgcgtaac

SEQ ID NO: 10 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 11 - protein, 15 aa, synthetic construct
RRRRRRRLLLLLLLL

SEQ ID NO: 12 - protein, 8 aa, synthetic construct
QQYYSYRT

SEQ ID NO: 13 - protein, 9 aa, synthetic construct
PCRKCACCA

SEQ ID NO: 14 - protein, 9 aa, synthetic construct
PRCCRWCCA

SEQ ID NO: 15 - protein, 9 aa, synthetic construct
PRRCKRCKC

SEQ ID NO: 16 - protein, 9 aa, synthetic construct
CKKCRKCCK

SEQ ID NO: 17 - protein, 9 aa, synthetic construct
CCRCKCWCC

SEQ ID NO: 18 - protein, 9 aa, synthetic construct
CCRKCCCCC

SEQ ID NO: 19 - protein, 9 aa, synthetic construct
PRKCCCCCC

SEQ ID NO: 20 - protein, 10 aa, synthetic construct
HHHHHHHHHH

SEQ ID NO: 21 - protein, 6 aa, synthetic construct
CCCCCC

SEQ ID NO: 22 - protein, 16 aa, synthetic construct
KRRRRQERAKEREKRR

SEQ ID NO: 23 - protein, 11 aa, synthetic construct
MRKTKALAPTA

SEQ ID NO: 24 - protein, 6 aa, synthetic construct
KKKRRP

SEQ ID NO: 25 - protein, 4 aa, synthetic construct
KKFK

SEQ ID NO: 26 - protein, 6 aa, synthetic construct
KKKKYN

SEQ ID NO: 27 - protein, 9 aa, synthetic construct
PPAKRERLD

SEQ ID NO: 28 - protein, 12 aa, synthetic construct
RGRGRRRRRRRR

SEQ ID NO: 29 - protein, 10 aa, synthetic construct
PKKNKLKKKS

SEQ ID NO: 30 - protein, 7 aa, synthetic construct
PKKKRKV

SEQ ID NO: 31 - protein, 20 aa, synthetic construct
NYKRPMDGTYGPPAKRHEGE

SEQ ID NO: 32 - protein, 8 aa, synthetic construct
KRSGSKAF

SEQ ID NO: 33 - protein, 9 aa, synthetic construct
PPAKRERLD

SEQ ID NO: 34 - protein, 18 aa, synthetic construct
RKKSGMQIALNDHLKQRR

SEQ ID NO: 35 - protein, 16 aa, synthetic construct
KKAFQNVLRIQCLCRK

SEQ ID NO: 36 - protein, 35 aa, synthetic construct
RRLLCRCGRRLPPEPCAAARPALFPSGVPAARSSP

SEQ ID NO: 37 - protein, 9 aa, synthetic construct
SVLGKRKFA

SEQ ID NO: 38 - DNA, 36 bp, synthetic construct
  source 1..36: mol_type = other DNA
cgatttccctttttccaccttagcaaccactaggac

SEQ ID NO: 39 - DNA, 41 bp, synthetic construct
  source 1..41: mol_type = other DNA
caccgttaccgttagcagcaagaagttaaaatccgttaaag

SEQ ID NO: 40 - DNA, 22 bp, synthetic construct
  source 1..22: mol_type = other DNA
aagaagttaaaatccgttaaag

SEQ ID NO: 41 - DNA, 18 bp, synthetic construct
  source 1..18: mol_type = other DNA
cgatttccctttttccac

SEQ ID NO: 42 - DNA, 38 bp, synthetic construct
  source 1..38: mol_type = other DNA
aggtggaaaaagggaaatcggatcgatggggatcccag

SEQ ID NO: 43 - DNA, 39 bp, synthetic construct
  source 1..39: mol_type = other DNA
ttaacggattttaacttcttgctgctaacggtaacggtg

SEQ ID NO: 44 - DNA, 54 bp, synthetic construct
  source 1..54: mol_type = other DNA
gggagcgcaggatccgctgccggttcaggagagtttgatcgatggggatcccag

SEQ ID NO: 45 - DNA, 72 bp, synthetic construct
  source 1..72: mol_type = other DNA
aaagagtcgggaagcgtctcttcggaacagttagcgcagttccgctcactggatgatcgatggggatcccag

SEQ ID NO: 46 - DNA, 38 bp, synthetic construct
  source 1..38: mol_type = other DNA
aggtggaaaaagggaaatcggggagcgcaggatccgct

SEQ ID NO: 47 - DNA, 38 bp, synthetic construct
  source 1..38: mol_type = other DNA
aggtggaaaaagggaaatcgaaagagtcgggaagcgtc

SEQ ID NO: 48 - DNA, 51 bp, synthetic construct
  source 1..51: mol_type = other DNA
gggagcgcaggatccgctgccggttcaggagagtttgatcgatggggatcc

SEQ ID NO: 49 - protein, 17 aa, synthetic construct
GSAGSAAGSGEFDRWGS

SEQ ID NO: 50 - DNA, 69 bp, synthetic construct
  source 1..69: mol_type = other DNA
aaagagtcgggaagcgtctcttcggaacagttagcgcagttccgctcactggatgatcgatggggatcc

SEQ ID NO: 51 - protein, 23 aa, synthetic construct
KESGSVSSEQLAQFRSLDDRWGS

SEQ ID NO: 52 - DNA, 3167 bp, synthetic construct
  source 1..3167: mol_type = other DNA
agtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatgctcaggctgctcttggctctcaacttattcccttcaattcaagtaacaggagggtcttcggactacaaggatcatgacggagactataaggatcacgatattgattacaaagatgacgacgacaaagacatccagatgacacagactacatcctccctgtctgcctctctgggagacagagtcaccatcagttgcagggcaagtcaggacatctctaagtatttgaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaggtaccaccacaacgcccgctcctcggccaccgacgccagcgccaactattgcgagtcagcctctcagtctgcgacctgaggcttgtcgaccagcagccggaggcgcagtgcacacgagggggctggacttcgcctgtgatagaagacctccttctaagcccttttgggtgctggtggtggttggtggagtcctggcttgctatagcttgctagtaacagtggcctttattattttctgggtgaggagtaagaggagcaggctcctgcacagtgactacatgaacatgactcccaggcggcccggacccacccgcaagcattaccagccctatgccccaccacgcgacttcgcagcctatcgctccgctagcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctagcgccacgaacttctctctgttaaagcaagcaggcgacgtggaagaaaaccccggtcccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgcca

SEQ ID NO: 53 - DNA, 363 bp, synthetic construct
  source 1..363: mol_type = other DNA
caggtgcagctgcaggagtctggaggaggcttggtgcagcctggggggtctctgagactctcctgtgcagcctctggattcacattcagtagctacgacatgagctgggtccgccaggctccggggaaggggctcgagtgggtctcaggtatgaatagtggtggtggtagaacatactatgaagactccgtgaagggccgattcaccatctccaggtccaacgccaagaacacgctgtatctgcaactgaacagcctgaaaactgacgacacggccatgtattactgtgtcacatccgactttgcttactggggccaggggacccaggtcaccgtctcctcatgttgttgttgttgttgttaa

SEQ ID NO: 54 - DNA, 390 bp, synthetic construct
  source 1..390: mol_type = other DNA
caggttcagctggaagaatctggtggtggtctggttcaggcgggtggttctctgcgtctgtcttgcgcggcgtctggtcgtacctcttctaacaccttcgttggttggttccgtcaggcgccgggtaaagaacgtgaattcgttgcggcgatccgtcgttctgacgaccgtacctactacgcggcgtctgttcgtggtcgtttcaccatctctggtgactctgcgaaaaacgttgttgcgctgcagatgtcttctctgcgtccggaagacaccgcggtttactactgcgcggcgacccgtacctggctggttaccggtcagtctgactacccgtactggggtcagggtacccaggttaccgtttcttcttgttgttgttgttgttgttaa

SEQ ID NO: 55 - DNA, 10609 bp, synthetic construct
  source 1..10609: mol_type = other DNA
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattccctctagaaataattttgtttaactttaagaaggagatataccatgcatcatcatcatcatcacagcagcggcagagaaaacttgtatttccagggcatgaacaacggcaccaacaactttcagaactttattggcattagcagcctgcagaaaaccctgcgcaacgcgctgattccgaccgaaaccacccagcagtttattgtgaaaaacggcattattaaagaagatgaactgcgcggcgaaaaccgccagattctgaaagatattatggatgattattatcgcggctttattagcgaaaccctgagcagcattgatgatattgattggaccagcctgtttgaaaaaatggaaattcagctgaaaaacggcgataacaaagataccctgattaaagaacagaccgaatatcgcaaagcgattcataaaaaatttgcgaacgatgatcgctttaaaaacatgtttagcgcgaaactgattagcgatattctgccggaatttgtgattcataacaacaactatagcgcgagcgaaaaagaagaaaaaacccaggtgattaaactgtttagccgctttgcgaccagctttaaagattattttaaaaaccgcgcgaactgctttagcgcggatgatattagcagcagcagctgccatcgcattgtgaacgataacgcggaaattttttttagcaacgcgctggtgtatcgccgcattgtgaaaagcctgagcaacgatgatattaacaaaattagcggcgatatgaaagatagcctgaaagaaatgagcctggaagaaatttatagctatgaaaaatatggcgaatttattacccaggaaggcattagcttttataacgatatttgcggcaaagtgaacagctttatgaacctgtattgccagaaaaacaaagaaaacaaaaacctgtataaactgcagaaactgcataaacagattctgtgcattgcggataccagctatgaagtgccgtataaatttgaaagcgatgaagaagtgtatcagagcgtgaacggctttctggataacattagcagcaaacatattgtggaacgcctgcgcaaaattggcgataactataacggctataacctggataaaatttatattgtgagcaaattttatgaaagcgtgagccagaaaacctatcgcgattgggaaaccattaacaccgcgctggaaattcattataacaacattctgccgggcaacggcaaaagcaaagcggataaagtgaaaaaagcggtgaaaaacgatctgcagaaaagcattaccgaaattaacgaactggtgagcaactataaactgtgcagcgatgataacattaaagcggaaacctatattcatgaaattagccatattctgaacaactttgaagcgcaggaactgaaatataacccggaaattcatctggtggaaagcgaactgaaagcgagcgaactgaaaaacgtgctggatgtgattatgaacgcgtttcattggtgcagcgtgtttatgaccgaagaactggtggataaagataacaacttttatgcggaactggaagaaatttatgatgaaatttatccggtgattagcctgtataacctggtgcgcaactatgtgacccagaaaccgtatagcaccaaaaaaattaaactgaactttggcattccgaccctggcggatggctggagcaaaagcaaagaatatagcaacaacgcgattattctgatgcgcgataacctgtattatctgggcatttttaacgcgaaaaacaaaccggataaaaaaattattgaaggcaacaccagcgaaaacaaaggcgattataaaaaaatgatttataacctgctgccgggcccgaacaaaatgattccgaaagtgtttctgagcagcaaaaccggcgtggaaacctataaaccgagcgcgtatattctggaaggctataaacagaacaaacatattaaaagcagcaaagattttgatattaccttttgccatgatctgattgattattttaaaaactgcattgcgattcatccggaatggaaaaactttggctttgattttagcgataccagcacctatgaagatattagcggcttttatcgcgaagtggaactgcagggctataaaattgattggacctatattagcgaaaaagatattgatctgctgcaggaaaaaggccagctgtatctgtttcagatttataacaaagattttagcaaaaaaagcaccggcaacgataacctgcataccatgtatctgaaaaacctgtttagcgaagaaaacctgaaagatattgtgctgaaactgaacggcgaagcggaaattttttttcgcaaaagcagcattaaaaacccgattattcataaaaaaggcagcattctggtgaaccgcacctatgaagcggaagaaaaagatcagtttggcaacattcagattgtgcgcaaaaacattccggaaaacatttatcaggaactgtataaatattttaacgataaaagcgataaagaactgagcgatgaagcggcgaaactgaaaaacgtggtgggccatcatgaagcggcgaccaacattgtgaaagattatcgctatacctatgataaatattttctgcatatgccgattaccattaactttaaagcgaacaaaaccggctttattaacgatcgcattctgcagtatattgcgaaagaaaaagatctgcatgtgattggcattgatcgcggcgaacgcaacctgatttatgtgagcgtgattgatacctgcggcaacattgtggaacagaaaagctttaacattgtgaacggctatgattatcagattaaactgaaacagcaggaaggcgcgcgccagattgcgcgcaaagaatggaaagaaattggcaaaattaaagaaattaaagaaggctatctgagcctggtgattcatgaaattagcaaaatggtgattaaatataacgcgattattgcgatggaagatctgagctatggctttaaaaaaggccgctttaaagtggaacgccaggtgtatcagaaatttgaaaccatgctgattaacaaactgaactatctggtgtttaaagatattagcattaccgaaaacggcggcctgctgaaaggctatcagctgacctatattccggataaactgaaaaacgtgggccatcagtgcggctgcattttttatgtgccggcggcgtataccagcaaaattgatccgaccaccggctttgtgaacatttttaaatttaaagatctgaccgtggatgcgaaacgcgaatttattaaaaaatttgatagcattcgctatgatagcgaaaaaaacctgttttgctttacctttgattataacaactttattacccagaacaccgtgatgagcaaaagcagctggagcgtgtatacctatggcgtgcgcattaaacgccgctttgtgaacggccgctttagcaacgaaagcgataccattgatattaccaaagatatggaaaaaaccctggaaatgaccgatattaactggcgcgatggccatgatctgcgccaggatattattgattatgaaattgtgcagcatatttttgaaatttttcgcctgaccgtgcagatgcgcaacagcctgagcgaactggaagatcgcgattatgatcgcctgattagcccggtgctgaacgaaaacaacattttttatgatagcgcgaaagcgggcgatgcgctgccgaaagatgcggatgcgaacggcgcgtattgcattgcgctgaaaggcctgtatgaaattaaacagattaccgaaaactggaaagaagatggcaaatttagccgcgataaactgaaaattagcaacaaagattggtttgattttattcagaacaaacgctatctgggcggcggcggcagcggcggcggcggcagcggcggcggcggcagcggcggcggcggcagcggcggcggcggcagcggcggcggcggcagcaccagccctaagaaaaaacgaaaagttgaggatcctaaaaagaaacgaaaagttcatcatcatcatcatcatgaatttgcgagcgcggaagcgggcattaccggcacctggtataaccagcatggcagcacctttaccgtgaccgcgggcgcggatggcaacctgaccggccagtatgaaaaccgcgcgcagggcaccggctgccagaacagcccgtataccctgaccggccgctataacggcaccaaactggaatggcgcgtggaatggaacaacagcaccgaaaactgccatagccgcaccgaatggcgcggccagtatcagggcggcgcggaagcgcgcattaacacccagtggaacctgacctatgaaggcggcagcggcccggcgaccgaacagggccaggatacctttaccaaagtgaaaccgagcgcggcgagcggcagcgattataaagatgatgatgataaaaaacgcaaaagaaaatgccgatatcctattggcattgacgtcaggtggcacttttcgaggagatcatgcacaggcggcggcggcagcggcggcggcggcagcggcggcggcggcagcggcggcggcggcagcggcggcggcggcagcggcggcagcccatggcaggttcagctggaagaatctggtggtggtctggttcaggcgggtggttctctgcgtctgtcttgcgcggcgtctggtcgtacctcttctaacaccttcgttggttggttccgtcaggcgccgggtaaagaacgtgaattcgttgcggcgatccgtcgttctgacgaccgtacctactacgcggcgtctgttcgtggtcgtttcaccatctctggtgactctgcgaaaaacgttgttgcgctgcagatgtcttctctgcgtccggaagacaccgcggtttactactgcgcggcgacccgtacctggctggttaccggtcagtctgactacccgtactggggtcagggtacccaggttaccgtttcttcttgttgttgttgttgttgttaagcggccgcactcgaggatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 56 - DNA, 10582 bp, synthetic construct
  source 1..10582: mol_type = other DNA
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattccctctagaaataattttgtttaactttaagaaggagatataccatgcatcatcatcatcatcacagcagcggcagagaaaacttgtatttccagggcatgaacaacggcaccaacaactttcagaactttattggcattagcagcctgcagaaaaccctgcgcaacgcgctgattccgaccgaaaccacccagcagtttattgtgaaaaacggcattattaaagaagatgaactgcgcggcgaaaaccgccagattctgaaagatattatggatgattattatcgcggctttattagcgaaaccctgagcagcattgatgatattgattggaccagcctgtttgaaaaaatggaaattcagctgaaaaacggcgataacaaagataccctgattaaagaacagaccgaatatcgcaaagcgattcataaaaaatttgcgaacgatgatcgctttaaaaacatgtttagcgcgaaactgattagcgatattctgccggaatttgtgattcataacaacaactatagcgcgagcgaaaaagaagaaaaaacccaggtgattaaactgtttagccgctttgcgaccagctttaaagattattttaaaaaccgcgcgaactgctttagcgcggatgatattagcagcagcagctgccatcgcattgtgaacgataacgcggaaattttttttagcaacgcgctggtgtatcgccgcattgtgaaaagcctgagcaacgatgatattaacaaaattagcggcgatatgaaagatagcctgaaagaaatgagcctggaagaaatttatagctatgaaaaatatggcgaatttattacccaggaaggcattagcttttataacgatatttgcggcaaagtgaacagctttatgaacctgtattgccagaaaaacaaagaaaacaaaaacctgtataaactgcagaaactgcataaacagattctgtgcattgcggataccagctatgaagtgccgtataaatttgaaagcgatgaagaagtgtatcagagcgtgaacggctttctggataacattagcagcaaacatattgtggaacgcctgcgcaaaattggcgataactataacggctataacctggataaaatttatattgtgagcaaattttatgaaagcgtgagccagaaaacctatcgcgattgggaaaccattaacaccgcgctggaaattcattataacaacattctgccgggcaacggcaaaagcaaagcggataaagtgaaaaaagcggtgaaaaacgatctgcagaaaagcattaccgaaattaacgaactggtgagcaactataaactgtgcagcgatgataacattaaagcggaaacctatattcatgaaattagccatattctgaacaactttgaagcgcaggaactgaaatataacccggaaattcatctggtggaaagcgaactgaaagcgagcgaactgaaaaacgtgctggatgtgattatgaacgcgtttcattggtgcagcgtgtttatgaccgaagaactggtggataaagataacaacttttatgcggaactggaagaaatttatgatgaaatttatccggtgattagcctgtataacctggtgcgcaactatgtgacccagaaaccgtatagcaccaaaaaaattaaactgaactttggcattccgaccctggcggatggctggagcaaaagcaaagaatatagcaacaacgcgattattctgatgcgcgataacctgtattatctgggcatttttaacgcgaaaaacaaaccggataaaaaaattattgaaggcaacaccagcgaaaacaaaggcgattataaaaaaatgatttataacctgctgccgggcccgaacaaaatgattccgaaagtgtttctgagcagcaaaaccggcgtggaaacctataaaccgagcgcgtatattctggaaggctataaacagaacaaacatattaaaagcagcaaagattttgatattaccttttgccatgatctgattgattattttaaaaactgcattgcgattcatccggaatggaaaaactttggctttgattttagcgataccagcacctatgaagatattagcggcttttatcgcgaagtggaactgcagggctataaaattgattggacctatattagcgaaaaagatattgatctgctgcaggaaaaaggccagctgtatctgtttcagatttataacaaagattttagcaaaaaaagcaccggcaacgataacctgcataccatgtatctgaaaaacctgtttagcgaagaaaacctgaaagatattgtgctgaaactgaacggcgaagcggaaattttttttcgcaaaagcagcattaaaaacccgattattcataaaaaaggcagcattctggtgaaccgcacctatgaagcggaagaaaaagatcagtttggcaacattcagattgtgcgcaaaaacattccggaaaacatttatcaggaactgtataaatattttaacgataaaagcgataaagaactgagcgatgaagcggcgaaactgaaaaacgtggtgggccatcatgaagcggcgaccaacattgtgaaagattatcgctatacctatgataaatattttctgcatatgccgattaccattaactttaaagcgaacaaaaccggctttattaacgatcgcattctgcagtatattgcgaaagaaaaagatctgcatgtgattggcattgatcgcggcgaacgcaacctgatttatgtgagcgtgattgatacctgcggcaacattgtggaacagaaaagctttaacattgtgaacggctatgattatcagattaaactgaaacagcaggaaggcgcgcgccagattgcgcgcaaagaatggaaagaaattggcaaaattaaagaaattaaagaaggctatctgagcctggtgattcatgaaattagcaaaatggtgattaaatataacgcgattattgcgatggaagatctgagctatggctttaaaaaaggccgctttaaagtggaacgccaggtgtatcagaaatttgaaaccatgctgattaacaaactgaactatctggtgtttaaagatattagcattaccgaaaacggcggcctgctgaaaggctatcagctgacctatattccggataaactgaaaaacgtgggccatcagtgcggctgcattttttatgtgccggcggcgtataccagcaaaattgatccgaccaccggctttgtgaacatttttaaatttaaagatctgaccgtggatgcgaaacgcgaatttattaaaaaatttgatagcattcgctatgatagcgaaaaaaacctgttttgctttacctttgattataacaactttattacccagaacaccgtgatgagcaaaagcagctggagcgtgtatacctatggcgtgcgcattaaacgccgctttgtgaacggccgctttagcaacgaaagcgataccattgatattaccaaagatatggaaaaaaccctggaaatgaccgatattaactggcgcgatggccatgatctgcgccaggatattattgattatgaaattgtgcagcatatttttgaaatttttcgcctgaccgtgcagatgcgcaacagcctgagcgaactggaagatcgcgattatgatcgcctgattagcccggtgctgaacgaaaacaacattttttatgatagcgcgaaagcgggcgatgcgctgccgaaagatgcggatgcgaacggcgcgtattgcattgcgctgaaaggcctgtatgaaattaaacagattaccgaaaactggaaagaagatggcaaatttagccgcgataaactgaaaattagcaacaaagattggtttgattttattcagaacaaacgctatctgggcggcggcggcagcggcggcggcggcagcggcggcggcggcagcggcggcggcggcagcggcggcggcggcagcggcggcggcggcagcaccagccctaagaaaaaacgaaaagttgaggatcctaaaaagaaacgaaaagttcatcatcatcatcatcatgaatttgcgagcgcggaagcgggcattaccggcacctggtataaccagcatggcagcacctttaccgtgaccgcgggcgcggatggcaacctgaccggccagtatgaaaaccgcgcgcagggcaccggctgccagaacagcccgtataccctgaccggccgctataacggcaccaaactggaatggcgcgtggaatggaacaacagcaccgaaaactgccatagccgcaccgaatggcgcggccagtatcagggcggcgcggaagcgcgcattaacacccagtggaacctgacctatgaaggcggcagcggcccggcgaccgaacagggccaggatacctttaccaaagtgaaaccgagcgcggcgagcggcagcgattataaagatgatgatgataaaaaacgcaaaagaaaatgccgatatcctattggcattgacgtcaggtggcacttttcgaggagatcatgcacaggcggcggcggcagcggcggcggcggcagcggcggcggcggcagcggcggcggcggcagcggcggcggcggcagcggcggcagcccatggcaggtgcagctgcaggagtctggaggaggcttggtgcagcctggggggtctctgagactctcctgtgcagcctctggattcacattcagtagctacgacatgagctgggtccgccaggctccggggaaggggctcgagtgggtctcaggtatgaatagtggtggtggtagaacatactatgaagactccgtgaagggccgattcaccatctccaggtccaacgccaagaacacgctgtatctgcaactgaacagcctgaaaactgacgacacggccatgtattactgtgtcacatccgactttgcttactggggccaggggacccaggtcaccgtctcctcatgttgttgttgttgttgttaagcggccgcactcgaggatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 57 - DNA, 10990 bp, synthetic construct
  source 1..10990: mol_type = other DNA
agcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgcaccatcaccatcaccatggaaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacactagtgaaaacctgtatttccagggagcagcctcgatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacgggtcacctaagaaaaaacgaaaagttgaggatcctaaaaagaaacgaaaagttgatagcggttcagagaccccaggaactagcgagagcgctacaccggaatcggcggaagcgggtatcaccggcacgtggtacaaccagtctggttctaccttcaccgttaccgcgggtgcggacggtaacctgaccggtcagtacgaaaaccgtgcgcagggcactggttgccagaactctccgtacaccctgaccggtcgttacaacggtaccaaactggaatggcgtgttgaatggaacaactctaccgaaaactgccactctcgtaccgaatggcgtggtcagtaccagggtggtgcggaagcgcgtatcaacacccagtggaacctgacctacgaaggtggttctggtccggcgaccgaacagggtcaggacaccttcaccaaagttaaaccgtctgcggcgtcttaagcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 58 - DNA, 11014 bp, synthetic construct
  source 1..11014: mol_type = other DNA
agcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgcaccatcaccatcaccatggaaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacactagtgaaaacctgtatttccagggagcagcctcgatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgcagcagtattatagctatcgcaccaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacgggtcacctaagaaaaaacgaaaagttgaggatcctaaaaagaaacgaaaagttgatagcggttcagagaccccaggaactagcgagagcgctacaccggaatcggcggaagcgggtatcaccggcacgtggtacaaccagtctggttctaccttcaccgttaccgcgggtgcggacggtaacctgaccggtcagtacgaaaaccgtgcgcagggcactggttgccagaactctccgtacaccctgaccggtcgttacaacggtaccaaactggaatggcgtgttgaatggaacaactctaccgaaaactgccactctcgtaccgaatggcgtggtcagtaccagggtggtgcggaagcgcgtatcaacacccagtggaacctgacctacgaaggtggttctggtccggcgaccgaacagggtcaggacaccttcaccaaagttaaaccgtctgcggcgtcttaagcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 59 - DNA, 24 bp, synthetic construct
  source 1..24: mol_type = other DNA
cagcagtattatagctatcgcacc

SEQ ID NO: 60 - protein, 8 aa, synthetic construct
QQYYSYRT

SEQ ID NO: 61 - DNA, 11530 bp, synthetic construct
  source 1..11530: mol_type = other DNA
agcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgcaccatcaccatcaccatggaaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacactagtgaaaacctgtatttccagggagcagcctcgatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgcagcagtattatagctatcgcaccaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacgggtcacctaagaaaaaacgaaaagttgaggatcctaaaaagaaacgaaaagttgatagcggttcagagaccccaggaactagcgagagcgctacaccggaatcggcggaagcgggtatcaccggcacgtggtacaaccagtctggttctaccttcaccgttaccgcgggtgcggacggtaacctgaccggtcagtacgaaaaccgtgcgcagggcactggttgccagaactctccgtacaccctgaccggtcgttacaacggtaccaaactggaatggcgtgttgaatggaacaactctaccgaaaactgccactctcgtaccgaatggcgtggtcagtaccagggtggtgcggaagcgcgtatcaacacccagtggaacctgacctacgaaggtggttctggtccggcgaccgaacagggtcaggacaccttcaccaaagttaaaggatcccaggttcagctggttcagagcggtggtggtctggttcaggcaggcggtagcctgcgtctgagctgtgcatttagcggtcgtacctttagcatgtataccatgggttggtttcgtcaggcaccgggtaaagaacgtgaatttgttgcagcaaatcgtggtcgtggtctgagtccggatattgcagatagcgttaatggtcgttttaccattagccgtgataatgccaaaaataccctgtacctgcagatggatagcctgaaaccggaagataccgcagtgtattattgtgcagcagcaagccgtgaagatccgcctggttattggggtcagggcaccaccgttaccgttagcagcccgaaaaagaaacgtaaagtggaagatccgaagaaaaagcgtaaagttggtggtggtggcagcctgccggaaaccggtggtctgtttgatattatcaagaaaattgccgagagcttcctggaacatcatcaccatcatcataagcttgatccggctgctaacaaagcccgaaaggaagctgagttggctgctgccaccgctgagcaataagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 62 - DNA, 570 bp, synthetic construct
  source 1..570: mol_type = other DNA
ggatcccaggttcagctggttcagagcggtggtggtctggttcaggcaggcggtagcctgcgtctgagctgtgcatttagcggtcgtacctttagcatgtataccatgggttggtttcgtcaggcaccgggtaaagaacgtgaatttgttgcagcaaatcgtggtcgtggtctgagtccggatattgcagatagcgttaatggtcgttttaccattagccgtgataatgccaaaaataccctgtacctgcagatggatagcctgaaaccggaagataccgcagtgtattattgtgcagcagcaagccgtgaagatccgcctggttattggggtcagggcaccaccgttaccgttagcagcccgaaaaagaaacgtaaagtggaagatccgaagaaaaagcgtaaagttggtggtggtggcagcctgccggaaaccggtggtctgtttgatattatcaagaaaattgccgagagcttcctggaacatcatcaccatcatcataagcttgatccggctgctaacaaagcccgaaaggaagctgagttggctgctgccaccgctgagcaataa

SEQ ID NO: 63 - protein, 8 aa, synthetic construct
QQYYSYRT

SEQ ID NO: 64 - DNA, 4938 bp, synthetic construct
  source 1..4938: mol_type = other DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcggatcgatggggatcccaggttcagctggttcagagcggtggtggtctggttcaggcaggcggtagcctgcgtctgagctgtgcatttagcggtcgtacctttagcatgtataccatgggttggtttcgtcaggcaccgggtaaagaacgtgaatttgttgcagcaaatcgtggtcgtggtctgagtccggatattgcagatagcgttaatggtcgttttaccattagccgtgataatgccaaaaataccctgtacctgcagatggatagcctgaaaccggaagataccgcagtgtattattgtgcagcagcaagccgtgaagatccgcctggttattggggtcagggcaccaccgttaccgttagcagcaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacgggtcacctaagaaaaaacgaaaagttgaggatcctaaaaagaaacgaaaagttgatagcggttcagagaccccaggaactagcgagagcgctacaccggaatcggcggaagcgggtatcaccggcacgtggtacaaccagtctggttctaccttcaccgttaccgcgggtgcggacggtaacctgaccggtcagtacgaaaaccgtgcgcagggcactggttgccagaactctccgtacaccctgaccggtcgttacaacggtaccaaactggaatggcgtgttgaatggaacaactctaccgaaaactgccactctcgtaccgaatggcgtggtcagtaccagggtggtgcggaagcgcgtatcaacacccagtggaacctgacctacgaaggtggttctggtccggcgaccgaacagggtcaggacaccttcaccaaagttaaaccgtctgcggcgtcttaa

SEQ ID NO: 65 - DNA, 6117 bp, synthetic construct
  source 1..6117: mol_type = other DNA
caccatcaccatcaccatggaaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacactagtgaaaacctgtatttccagggagcagcctcgatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcggatcgatggggatcccaggttcagctggttcagagcggtggtggtctggttcaggcaggcggtagcctgcgtctgagctgtgcatttagcggtcgtacctttagcatgtataccatgggttggtttcgtcaggcaccgggtaaagaacgtgaatttgttgcagcaaatcgtggtcgtggtctgagtccggatattgcagatagcgttaatggtcgttttaccattagccgtgataatgccaaaaataccctgtacctgcagatggatagcctgaaaccggaagataccgcagtgtattattgtgcagcagcaagccgtgaagatccgcctggttattggggtcagggcaccaccgttaccgttagcagcaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacgggtcacctaagaaaaaacgaaaagttgaggatcctaaaaagaaacgaaaagttgatagcggttcagagaccccaggaactagcgagagcgctacaccggaatcggcggaagcgggtatcaccggcacgtggtacaaccagtctggttctaccttcaccgttaccgcgggtgcggacggtaacctgaccggtcagtacgaaaaccgtgcgcagggcactggttgccagaactctccgtacaccctgaccggtcgttacaacggtaccaaactggaatggcgtgttgaatggaacaactctaccgaaaactgccactctcgtaccgaatggcgtggtcagtaccagggtggtgcggaagcgcgtatcaacacccagtggaacctgacctacgaaggtggttctggtccggcgaccgaacagggtcaggacaccttcaccaaagttaaaccgtctgcggcgtcttaa

SEQ ID NO: 66 - protein, 1645 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSDRWGSQVQLVQSGGGLVQAGGSLRLSCAFSGRTFSMYTMGWFRQAPGKEREFVAANRGRGLSPDIADSVNGRFTISRDNAKNTLYLQMDSLKPEDTAVYYCAAASREDPPGYWGQGTTVTVSSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVEDPKKKRKVDSGSETPGTSESATPESAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAAS

SEQ ID NO: 67 - DNA, 6153 bp, synthetic construct
  source 1..6153: mol_type = other DNA
caccatcaccatcaccatggaaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacactagtgaaaacctgtatttccagggagcagcctcgatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcggggagcgcaggatccgctgccggttcaggagagtttgatcgatggggatcccaggttcagctggttcagagcggtggtggtctggttcaggcaggcggtagcctgcgtctgagctgtgcatttagcggtcgtacctttagcatgtataccatgggttggtttcgtcaggcaccgggtaaagaacgtgaatttgttgcagcaaatcgtggtcgtggtctgagtccggatattgcagatagcgttaatggtcgttttaccattagccgtgataatgccaaaaataccctgtacctgcagatggatagcctgaaaccggaagataccgcagtgtattattgtgcagcagcaagccgtgaagatccgcctggttattggggtcagggcaccaccgttaccgttagcagcaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacgggtcacctaagaaaaaacgaaaagttgaggatcctaaaaagaaacgaaaagttgatagcggttcagagaccccaggaactagcgagagcgctacaccggaatcggcggaagcgggtatcaccggcacgtggtacaaccagtctggttctaccttcaccgttaccgcgggtgcggacggtaacctgaccggtcagtacgaaaaccgtgcgcagggcactggttgccagaactctccgtacaccctgaccggtcgttacaacggtaccaaactggaatggcgtgttgaatggaacaactctaccgaaaactgccactctcgtaccgaatggcgtggtcagtaccagggtggtgcggaagcgcgtatcaacacccagtggaacctgacctacgaaggtggttctggtccggcgaccgaacagggtcaggacaccttcaccaaagttaaaccgtctgcggcgtcttaa

SEQ ID NO: 68 - DNA, 6153 bp, synthetic construct; identical to SEQ ID NO: 67
  source 1..6153: mol_type = other DNA

SEQ ID NO: 69 - protein, 1657 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSGSAGSAAGSGEFDRWGSQVQLVQSGGGLVQAGGSLRLSCAFSGRTFSMYTMGWFRQAPGKEREFVAANRGRGLSPDIADSVNGRFTISRDNAKNTLYLQMDSLKPEDTAVYYCAAASREDPPGYWGQGTTVTVSSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVEDPKKKRKVDSGSETPGTSESATPESAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAAS

SEQ ID NO: 70 - DNA, 4992 bp, synthetic construct
  source 1..4992: mol_type = other DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaaagagtcgggaagcgtctcttcggaacagttagcgcagttccgctcactggatgatcgatggggatcccaggttcagctggttcagagcggtggtggtctggttcaggcaggcggtagcctgcgtctgagctgtgcatttagcggtcgtacctttagcatgtataccatgggttggtttcgtcaggcaccgggtaaagaacgtgaatttgttgcagcaaatcgtggtcgtggtctgagtccggatattgcagatagcgttaatggtcgttttaccattagccgtgataatgccaaaaataccctgtacctgcagatggatagcctgaaaccggaagataccgcagtgtattattgtgcagcagcaagccgtgaagatccgcctggttattggggtcagggcaccaccgttaccgttagcagcaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacgggtcacctaagaaaaaacgaaaagttgaggatcctaaaaagaaacgaaaagttgatagcggttcagagaccccaggaactagcgagagcgctacaccggaatcggcggaagcgggtatcaccggcacgtggtacaaccagtctggttctaccttcaccgttaccgcgggtgcggacggtaacctgaccggtcagtacgaaaaccgtgcgcagggcactggttgccagaactctccgtacaccctgaccggtcgttacaacggtaccaaactggaatggcgtgttgaatggaacaactctaccgaaaactgccactctcgtaccgaatggcgtggtcagtaccagggtggtgcggaagcgcgtatcaacacccagtggaacctgacctacgaaggtggttctggtccggcgaccgaacagggtcaggacaccttcaccaaagttaaaccgtctgcggcgtcttaa

SEQ ID NO: 71 - DNA, 6171 bp, synthetic construct
  source 1..6171: mol_type = other DNA
caccatcaccatcaccatggaaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacactagtgaaaacctgtatttccagggagcagcctcgatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaaagagtcgggaagcgtctcttcggaacagttagcgcagttccgctcactggatgatcgatggggatcccaggttcagctggttcagagcggtggtggtctggttcaggcaggcggtagcctgcgtctgagctgtgcatttagcggtcgtacctttagcatgtataccatgggttggtttcgtcaggcaccgggtaaagaacgtgaatttgttgcagcaaatcgtggtcgtggtctgagtccggatattgcagatagcgttaatggtcgttttaccattagccgtgataatgccaaaaataccctgtacctgcagatggatagcctgaaaccggaagataccgcagtgtattattgtgcagcagcaagccgtgaagatccgcctggttattggggtcagggcaccaccgttaccgttagcagcaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacgggtcacctaagaaaaaacgaaaagttgaggatcctaaaaagaaacgaaaagttgatagcggttcagagaccccaggaactagcgagagcgctacaccggaatcggcggaagcgggtatcaccggcacgtggtacaaccagtctggttctaccttcaccgttaccgcgggtgcggacggtaacctgaccggtcagtacgaaaaccgtgcgcagggcactggttgccagaactctccgtacaccctgaccggtcgttacaacggtaccaaactggaatggcgtgttgaatggaacaactctaccgaaaactgccactctcgtaccgaatggcgtggtcagtaccagggtggtgcggaagcgcgtatcaacacccagtggaacctgacctacgaaggtggttctggtccggcgaccgaacagggtcaggacaccttcaccaaagttaaaccgtctgcggcgtcttaa

SEQ ID NO: 72 - protein, 1663 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKESGSVSSEQLAQFRSLDDRWGSQVQLVQSGGGLVQAGGSLRLSCAFSGRTFSMYTMGWFRQAPGKEREFVAANRGRGLSPDIADSVNGRFTISRDNAKNTLYLQMDSLKPEDTAVYYCAAASREDPPGYWGQGTTVTVSSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVEDPKKKRKVDSGSETPGTSESATPESAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAAS

SEQ ID NO: 73 - DNA, 11014 bp, synthetic construct; identical to SEQ ID NO: 58
  source 1..11014: mol_type = other DNA

SEQ ID NO: 74 - protein, 1531 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSMRKGYAMDYKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVEDPKKKRKVDSGSETPGTSESATPESAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAAS

SEQ ID NO: 75 - protein, 12 aa, synthetic construct
SVGWAVITDEYK

SEQ ID NO: 76 - protein, 19 aa, synthetic construct
KNLIGALLFDSGETAEATR

SEQ ID NO: 77 - protein, 18 aa, synthetic construct
NLIGALLFDSGETAEATR

SEQ ID NO: 78 - protein, 17 aa, synthetic construct
LIGALLFDSGETAEATR

SEQ ID NO: 79 - protein, 16 aa, synthetic construct
IGALLFDSGETAEATR

SEQ ID NO: 80 - protein, 14 aa, synthetic construct
ALLFDSGETAEATR

SEQ ID NO: 81 - protein, 13 aa, synthetic construct
LLFDSGETAEATR

SEQ ID NO: 82 - protein, 12 aa, synthetic construct
LFDSGETAEATR

SEQ ID NO: 83 - protein, 11 aa, synthetic construct
FDSGETAEATR

SEQ ID NO: 84 - protein, 14 aa, synthetic construct
ICYLQEIFSNEMAK

SEQ ID NO: 85 - protein, 8 aa, synthetic construct
VDDSFFHR

SEQ ID NO: 86 - protein, 9 aa, synthetic construct
VDDSFFHRL

SEQ ID NO: 87 - protein, 11 aa, synthetic construct
LEESFLVEEDK

SEQ ID NO: 88 - protein, 12 aa, synthetic construct
LEESFLVEEDKK

SEQ ID NO: 89 - protein, 15 aa, synthetic construct
LEESFLVEEDKKHER

SEQ ID NO: 90 - protein, 16 aa, synthetic construct
HPIFGNIVDEVAYHEK

SEQ ID NO: 91 - protein, 15 aa, synthetic construct
PIFGNIVDEVAYHEK

SEQ ID NO: 92 - protein, 14 aa, synthetic construct
IFGNIVDEVAYHEK

SEQ ID NO: 93 - protein, 10 aa, synthetic construct
IVDEVAYHEK

SEQ ID NO: 94 - protein, 8 aa, synthetic construct
YPTIYHLR

SEQ ID NO: 95 - protein, 12 aa, synthetic construct
KLVDSTDKADLR

SEQ ID NO: 96 - protein, 11 aa, synthetic construct
LVDSTDKADLR

SEQ ID NO: 97 - protein, 11 aa, synthetic construct
LIYLALAHMIK

SEQ ID NO: 98 - protein, 18 aa, synthetic construct
GHFLIEGDLNPDNSDVDK

SEQ ID NO: 99 - protein, 16 aa, synthetic construct
FLIEGDLNPDNSDVDK

SEQ ID NO: 100 - protein, 22 aa, synthetic construct
LVQTYNQLFEENPINASGVDAK

SEQ ID NO: 101 - protein, 17 aa, synthetic construct
NQLFEENPINASGVDAK

SEQ ID NO: 102 - protein, 16 aa, synthetic construct
QLFEENPINASGVDAK

SEQ ID NO: 103 - protein, 13 aa, synthetic construct
RLENLIAQLPGEK

SEQ ID NO: 104 - protein, 14 aa, synthetic construct
RLENLIAQLPGEKK

SEQ ID NO: 105 - protein, 12 aa, synthetic construct
LENLIAQLPGEK

SEQ ID NO: 106 - protein, 13 aa, synthetic construct
LENLIAQLPGEKK

SEQ ID NO: 107 - protein, 13 aa, synthetic construct
LIALSLGLTPNFK

SEQ ID NO: 108 - protein, 12 aa, synthetic construct
FKSNFDLAEDAK

SEQ ID NO: 109 - protein, 10 aa, synthetic construct
SNFDLAEDAK

SEQ ID NO: 110 - protein, 16 aa, synthetic construct
AAKNLSDAILLSDILR

SEQ ID NO: 111 - protein, 13 aa, synthetic construct
NLSDAILLSDILR

SEQ ID NO: 112 - protein, 16 aa, synthetic construct
VNTEITKAPLSASMIK

SEQ ID NO: 113 - protein, 9 aa, synthetic construct
APLSASMIK

SEQ ID NO: 114 - protein, 13 aa, synthetic construct
RYDEHHQDLTLLK

SEQ ID NO: 115 - protein, 12 aa, synthetic construct
YDEHHQDLTLLK

SEQ ID NO: 116 - protein, 10 aa, synthetic construct
YKEIFFDQSK

SEQ ID NO: 117 - protein, 8 aa, synthetic construct
EIFFDQSK

SEQ ID NO: 118 - protein, 18 aa, synthetic construct
NGYAGYIDGGASQEEFYK

SEQ ID NO: 119 - protein, 10 aa, synthetic construct
GGASQEEFYK

SEQ ID NO: 120 - protein, 8 aa, synthetic construct
FIKPILEK

SEQ ID NO: 121 - protein, 18 aa, synthetic construct
FIKPILEKMDGTEELLVK

SEQ ID NO: 122 - protein, 10 aa, synthetic construct
MDGTEELLVK

SEQ ID NO: 123 - protein, 21 aa, synthetic construct
TFDNGSIPHQIHLGELHAILR

SEQ ID NO: 124 - protein, 12 aa, synthetic construct
QIHLGELHAILR

SEQ ID NO: 125 - protein, 8 aa, synthetic construct
RQEDFYPF

SEQ ID NO: 126 - protein, 10 aa, synthetic construct
RQEDFYPFLK

SEQ ID NO: 127 - protein, 10 aa, synthetic construct
RQEDFYPFLK

SEQ ID NO: 128 - protein, 9 aa, synthetic construct
QEDFYPFLK

SEQ ID NO: 129 - protein, 12 aa, synthetic construct
QEDFYPFLKDNR

SEQ ID NO: 130 - protein, 10 aa, synthetic construct
IPYYVGPLAR

SEQ ID NO: 131 - protein, 8 aa, synthetic construct
YYVGPLAR

SEQ ID NO: 132 - protein, 6 aa, synthetic construct
FAWMTR

SEQ ID NO: 133 - protein, 17 aa, synthetic construct
KSEETITPWNFEEVVDK

SEQ ID NO: 134 - protein, 16 aa, synthetic construct
SEETITPWNFEEVVDK

SEQ ID NO: 135 - protein, 18 aa, synthetic construct
SEETITPWNFEEVVDKGA

SEQ ID NO: 136 - protein, 18 aa, synthetic construct
NFEEVVDKGASAQSFIER

SEQ ID NO: 137 - protein, 10 aa, synthetic construct
GASAQSFIER

SEQ ID NO: 138 - protein, 8 aa, synthetic construct
SAQSFIER

SEQ ID NO: 139 - protein, 12 aa, synthetic construct
MTNFDKNLPNEK

SEQ ID NO: 140 - protein, 16 aa, synthetic construct
HSLLYEYFTVYNELTK

SEQ ID NO: 141 - protein, 7 aa, synthetic construct
YVTEGMR

SEQ ID NO: 142 - protein, 10 aa, synthetic construct
KPAFLSGEQK

SEQ ID NO: 143 - protein, 9 aa, synthetic construct
PAFLSGEQK

SEQ ID NO: 144 - protein, 9 aa, synthetic construct
KAIVDLLFK

SEQ ID NO: 145 - protein, 8 aa, synthetic construct
QLKEDYFK

SEQ ID NO: 146 - protein, 16 aa, synthetic construct
KIECFDSVEISGVEDR

SEQ ID NO: 147 - protein, 15 aa, synthetic construct
IECFDSVEISGVEDR

SEQ ID NO: 148 - protein, 12 aa, synthetic construct
FDSVEISGVEDR

SEQ ID NO: 149 - protein, 13 aa, synthetic construct
FNASLGTYHDLLK

SEQ ID NO: 150 - protein, 11 aa, synthetic construct
ASLGTYHDLLK

SEQ ID NO: 151 - protein, 27 aa, synthetic construct
DKDFLDNEENEDILEDIVLTLTLFEDR

SEQ ID NO: 152 - protein, 6 aa, synthetic construct
EMIEER

SEQ ID NO: 153 - protein, 11 aa, synthetic construct
LKTYAHLFDDK

SEQ ID NO: 154 - protein, 12 aa, synthetic construct
TYAHLFDDKVMK

SEQ ID NO: 155 - protein, 14 aa, synthetic construct
NFMQLIHDDSLTFK

SEQ ID NO: 156 - protein, 19 aa, synthetic construct
NFMQLIHDDSLTFKEDIQK

SEQ ID NO: 157 - protein, 12 aa, synthetic construct
AQVSGQGDSLHE

SEQ ID NO: 158 - protein, 13 aa, synthetic construct
AQVSGQGDSLHEH

SEQ ID NO: 159 - protein, 15 aa, synthetic construct
AQVSGQGDSLHEHIA

SEQ ID NO: 160 - protein, 16 aa, synthetic construct
AQVSGQGDSLHEHIAN

SEQ ID NO: 161 - protein, 24 aa, synthetic construct
AQVSGQGDSLHEHIANLAGSPAIK

SEQ ID NO: 162 - protein, 25 aa, synthetic construct
AQVSGQGDSLHEHIANLAGSPAIKK

SEQ ID NO: 163 - protein, 12 aa, synthetic construct
HKPENIVIEMAR

SEQ ID NO: 164 - protein, 14 aa, synthetic construct
IEEGIKELGSQILK

SEQ ID NO: 165 - protein, 13 aa, synthetic construct
EHPVENTQLQNEK

SEQ ID NO: 166 - protein, 10 aa, synthetic construct
LYLYYLQNGR

SEQ ID NO: 167 - protein, 12 aa, synthetic construct
DMYVDQELDINR

SEQ ID NO: 168 - protein, 16 aa, synthetic construct
LSDYDVDHIVPQSFLK

SEQ ID NO: 169 - protein, 11 aa, synthetic construct
SDNVPSEEVVK

SEQ ID NO: 170 - protein, 9 aa, synthetic construct
HVAQILDSR

SEQ ID NO: 171 - protein, 9 aa, synthetic construct
YDENDKLIR

SEQ ID NO: 172 - protein, 11 aa, synthetic construct
LESEFVYGDYK

SEQ ID NO: 173 - protein, 11 aa, synthetic construct
KTEVQTGGFSK

SEQ ID NO: 174 - protein, 13 aa, synthetic construct
GLSPDIADSVNGR

SEQ ID NO: 175 - protein, 19 aa, synthetic construct
EVLDATLIHQSITGLYETR

SEQ ID NO: 176 - protein, 12 aa, synthetic construct
SVGWAVITDEYK

SEQ ID NO: 177 - protein, 19 aa, synthetic construct
KNLIGALLFDSGETAEATR

SEQ ID NO: 178 - protein, 18 aa, synthetic construct
NLIGALLFDSGETAEATR

SEQ ID NO: 179 - protein, 17 aa, synthetic construct
LIGALLFDSGETAEATR

SEQ ID NO: 180 - protein, 16 aa, synthetic construct
IGALLFDSGETAEATR

SEQ ID NO: 181 - protein, 14 aa, synthetic construct
ALLFDSGETAEATR

SEQ ID NO: 182 - protein, 13 aa, synthetic construct
LLFDSGETAEATR

SEQ ID NO: 183 - protein, 11 aa, synthetic construct
FDSGETAEATR

SEQ ID NO: 184 - protein, 14 aa, synthetic construct
ICYLQEIFSNEMAK

SEQ ID NO: 185 - protein, 14 aa, synthetic construct
ICYLQEIFSNEMAK

SEQ ID NO: 186 - protein, 8 aa, synthetic construct
VDDSFFHR

SEQ ID NO: 187 - protein, 9 aa, synthetic construct
VDDSFFHRL

SEQ ID NO: 188 - protein, 12 aa, synthetic construct
VDDSFFHRLEES

SEQ ID NO: 189 - protein, 7 aa, synthetic construct
DDSFFHR

SEQ ID NO: 190 - protein, 11 aa, synthetic construct
LEESFLVEEDK

SEQ ID NO: 191 - protein, 15 aa, synthetic construct
LEESFLVEEDKKHER

SEQ ID NO: 192 - protein, 11 aa, synthetic construct
FLVEEDKKHER

SEQ ID NO: 193 - protein, 12 aa, synthetic construct
HPIFGNIVDEVA

SEQ ID NO: 194 - protein, 13 aa, synthetic construct
HPIFGNIVDEVAY

SEQ ID NO: 195 - protein, 16 aa, synthetic construct
HPIFGNIVDEVAYHEK

SEQ ID NO: 196 - protein, 15 aa, synthetic construct
PIFGNIVDEVAYHEK

SEQ ID NO: 197 - protein, 8 aa, synthetic construct
YPTIYHLR

SEQ ID NO: 198 - protein, 12 aa, synthetic construct
KLVDSTDKADLR

SEQ ID NO: 199 - protein, 11 aa, synthetic construct
LVDSTDKADLR

SEQ ID NO: 200 - protein, 8 aa, synthetic construct
LIYLALAH

SEQ ID NO: 201 - protein, 11 aa, synthetic construct
LIYLALAHMIK

SEQ ID NO: 202 - protein, 18 aa, synthetic construct
GHFLIEGDLNPDNSDVDK

SEQ ID NO: 203 - protein, 17 aa, synthetic construct
NQLFEENPINASGVDAK

SEQ ID NO: 204 - protein, 16 aa, synthetic construct
QLFEENPINASGVDAK

SEQ ID NO: 205 - protein, 11 aa, synthetic construct
RLENLIAQLPG

SEQ ID NO: 206 - protein, 13 aa, synthetic construct
RLENLIAQLPGEK

SEQ ID NO: 207 - protein, 14 aa, synthetic construct
RLENLIAQLPGEKK

SEQ ID NO: 208 - protein, 12 aa, synthetic construct
LENLIAQLPGEK

SEQ ID NO: 209 - protein, 13 aa, synthetic construct
LENLIAQLPGEKK

SEQ ID NO: 210 - protein, 9 aa, synthetic construct
LIAQLPGEK

SEQ ID NO: 211 - protein, 13 aa, synthetic construct
LIALSLGLTPNFK

SEQ ID NO: 212 - protein, 10 aa, synthetic construct
LSLGLTPNFK

SEQ ID NO: 213 - protein, 10 aa, synthetic construct
SNFDLAEDAK

SEQ ID NO: 214 - protein, 12 aa, synthetic construct
SNFDLAEDAKLQ

SEQ ID NO: 215 - protein, 15 aa, synthetic construct
SNFDLAEDAKLQLSK

SEQ ID NO: 216 - protein, 13 aa, synthetic construct
IGDQYADLFLAAK

SEQ ID NO: 217 - protein, 11 aa, synthetic construct
DQYADLFLAAK

SEQ ID NO: 218 - protein, 13 aa, synthetic construct
NLSDAILLSDILR

SEQ ID NO: 219 - protein, 12 aa, synthetic construct
LSDAILLSDILR

SEQ ID NO: 220 - protein, 11 aa, synthetic construct
SDAILLSDILR

SEQ ID NO: 221 - protein, 9 aa, synthetic construct
AILLSDILR

SEQ ID NO: 222 - protein, 9 aa, synthetic construct
APLSASMIK

SEQ ID NO: 223 - protein, 10 aa, synthetic construct
APLSASMIKR

SEQ ID NO: 224 - protein, 13 aa, synthetic construct
RYDEHHQDLTLLK

SEQ ID NO: 225 - protein, 12 aa, synthetic construct
YDEHHQDLTLLK

SEQ ID NO: 226 - protein, 8 aa, synthetic construct
HQDLTLLK

SEQ ID NO: 227 - protein, 8 aa, synthetic construct
QQLPEKYK

SEQ ID NO: 228 - protein, 16 aa, synthetic construct
QQLPEKYKEIFFDQSK

SEQ ID NO: 229 - protein, 10 aa, synthetic construct
YKEIFFDQSK

SEQ ID NO: 230 - protein, 8 aa, synthetic construct
EIFFDQSK

SEQ ID NO: 231 - protein, 18 aa, synthetic construct
NGYAGYIDGGASQEEFYK

SEQ ID NO: 232 - protein, 17 aa, synthetic construct
GYAGYIDGGASQEEFYK

SEQ ID NO: 233 - protein, 16 aa, synthetic construct
YAGYIDGGASQEEFYK

SEQ ID NO: 234 - protein, 12 aa, synthetic construct
IDGGASQEEFYK

SEQ ID NO: 235 - protein, 8 aa, synthetic construct
FIKPILEK

SEQ ID NO: 236 - protein, 18 aa, synthetic construct
FIKPILEKMDGTEELLVK

SEQ ID NO: 237 - protein, 10 aa, synthetic construct
MDGTEELLVK

SEQ ID NO: 238 - protein, 21 aa, synthetic construct
TFDNGSIPHQIHLGELHAILR

SEQ ID NO: 239 - protein, 12 aa, synthetic construct
QIHLGELHAILR

SEQ ID NO: 240 - protein, 8 aa, synthetic construct
RQEDFYPF

SEQ ID NO: 241 - protein, 10 aa, synthetic construct
RQEDFYPFLK

SEQ ID NO: 242 - protein, 13 aa, synthetic construct
RQEDFYPFLKDNR

SEQ ID NO: 243 - protein, 9 aa, synthetic construct
QEDFYPFLK

SEQ ID NO: 244 - protein, 12 aa, synthetic construct
QEDFYPFLKDNR

SEQ ID NO: 245 - protein, 15 aa, synthetic construct
ILTFRIPYYVGPLAR

SEQ ID NO: 246 - protein, 10 aa, synthetic construct
IPYYVGPLAR

SEQ ID NO: 247 - protein, 6 aa, synthetic construct
FAWMTR

SEQ ID NO: 248 - protein, 17 aa, synthetic construct
KSEETITPWNFEEVVDK

SEQ ID NO: 249 - protein, 22 aa, synthetic construct
KSEETITPWNFEEVVDKGASAQ

SEQ ID NO: 250 - protein, 27 aa, synthetic construct
KSEETITPWNFEEVVDKGASAQSFIER

SEQ ID NO: 251 - protein, 16 aa, synthetic construct
SEETITPWNFEEVVDK

SEQ ID NO: 252 - protein, 18 aa, synthetic construct
NFEEVVDKGASAQSFIER

SEQ ID NO: 253 - protein, 10 aa, synthetic construct
GASAQSFIER

SEQ ID NO: 254 - protein, 12 aa, synthetic construct
MTNFDKNLPNEK

SEQ ID NO: 255 - protein, 9 aa, synthetic construct
FDKNLPNEK

SEQ ID NO: 256 - protein, 16 aa, synthetic construct
HSLLYEYFTVYNELTK

SEQ ID NO: 257 - protein, 7 aa, synthetic construct
YVTEGMR

SEQ ID NO: 258 - protein, 10 aa, synthetic construct
KPAFLSGEQK

SEQ ID NO: 259 - protein, 11 aa, synthetic construct
KPAFLSGEQKK

SEQ ID NO: 260 - protein, 9 aa, synthetic construct
KAIVDLLFK

SEQ ID NO: 261 - protein, 8 aa, synthetic construct
AIVDLLFK

SEQ ID NO: 262 - protein, 8 aa, synthetic construct
QLKEDYFK

SEQ ID NO: 263 - protein, 9 aa, synthetic construct
QLKEDYFKK

SEQ ID NO: 264 - protein, 16 aa, synthetic construct
KIECFDSVEISGVEDR

SEQ ID NO: 265 - protein, 15 aa, synthetic construct
IECFDSVEISGVEDR

SEQ ID NO: 266 - protein, 12 aa, synthetic construct
FDSVEISGVEDR

SEQ ID NO: 267 - protein, 10 aa, synthetic construct
SVEISGVEDR

SEQ ID NO: 268 - protein, 13 aa, synthetic construct
FNASLGTYHDLLK

SEQ ID NO: 269 - protein, 11 aa, synthetic construct
ASLGTYHDLLK

SEQ ID NO: 270 - protein, 27 aa, synthetic construct
DKDFLDNEENEDILEDIVLTLTLFEDR

SEQ ID NO: 271 - protein, 25 aa, synthetic construct
DFLDNEENEDILEDIVLTLTLFEDR

SEQ ID NO: 272 - protein, 11 aa, synthetic construct
LKTYAHLFDDK

SEQ ID NO: 273 - protein, 12 aa, synthetic construct
TYAHLFDDKVMK

SEQ ID NO: 274 - protein, 7 aa, synthetic construct
RYTGWGR

SEQ ID NO: 275 - protein, 7 aa, synthetic construct
TILDFLK

SEQ ID NO: 276 - protein, 14 aa, synthetic construct
TILDFLKSDGFANR

SEQ ID NO: 277 - protein, 12 aa, synthetic construct
NFMQLIHDDSLT

SEQ ID NO: 278 - protein, 14 aa, synthetic construct
NFMQLIHDDSLTFK

SEQ ID NO: 279 - protein, 19 aa, synthetic construct
NFMQLIHDDSLTFKEDIQK

SEQ ID NO: 280 - protein, 15 aa, synthetic construct
AQVSGQGDSLHEHIA

SEQ ID NO: 281 - protein, 24 aa, synthetic construct
AQVSGQGDSLHEHIANLAGSPAIK

SEQ ID NO: 282 - protein, 25 aa, synthetic construct
AQVSGQGDSLHEHIANLAGSPAIKK

SEQ ID NO: 283 - protein, 14 aa, synthetic construct
GILQTVKVVDELVK

SEQ ID NO: 284 - protein, 12 aa, synthetic construct
HKPENIVIEMAR

SEQ ID NO: 285 - protein, 11 aa, synthetic construct
KPENIVIEMAR

SEQ ID NO: 286 - protein, 13 aa, synthetic construct
IEEGIKELGSQIL

SEQ ID NO: 287 - protein, 14 aa, synthetic construct
IEEGIKELGSQILK

SEQ ID NO: 288 - protein, 13 aa, synthetic construct
EHPVENTQLQNEK

SEQ ID NO: 289 - protein, 10 aa, synthetic construct
LYLYYLQNGR

SEQ ID NO: 290 - protein, 18 aa, synthetic construct
YLQNGRDMYVDQELDINR

SEQ ID NO: 291 - protein, 12 aa, synthetic construct
DMYVDQELDINR

SEQ ID NO: 292 - protein, 12 aa, synthetic construct
LSDYDVDHIVPQ

SEQ ID NO: 293 - protein, 13 aa, synthetic construct
LSDYDVDHIVPQS

SEQ ID NO: 294 - protein, 14 aa, synthetic construct
LSDYDVDHIVPQSF

SEQ ID NO: 295 - protein, 16 aa, synthetic construct
LSDYDVDHIVPQSFLK

SEQ ID NO: 296 - protein, 23 aa, synthetic construct
LSDYDVDHIVPQSFLKDDSIDNK

SEQ ID NO: 297 - protein, 11 aa, synthetic construct
SFLKDDSIDNK

SEQ ID NO: 298 - protein, 11 aa, synthetic construct
DDSIDNKVLTR

SEQ ID NO: 299 - protein, 13 aa, synthetic construct
GKSDNVPSEEVVK

SEQ ID NO: 300 - protein, 11 aa, synthetic construct
SDNVPSEEVVK

SEQ ID NO: 301 - protein, 7 aa, synthetic construct
KFDNLTK

SEQ ID NO: 302 - protein, 11 aa, synthetic construct
GGLSELDKAGF

SEQ ID NO: 303 - protein, 13 aa, synthetic construct
GGLSELDKAGFIK

SEQ ID NO: 304 - protein, 9 aa, synthetic construct
HVAQILDSR

SEQ ID NO: 305 - protein, 8 aa, synthetic construct
VAQILDSR

SEQ ID NO: 306 - protein, 13 aa, synthetic construct
MNTKYDENDKLIR

SEQ ID NO: 307 - protein, 9 aa, synthetic construct
YDENDKLIR

SEQ ID NO: 308 - protein, 23 aa, synthetic construct
EINNYHHAHDAYLNAVVGTALIK

SEQ ID NO: 309 - protein, 14 aa, synthetic construct
DAYLNAVVGTALIK

SEQ ID NO: 310 - protein, 11 aa, synthetic construct
LESEFVYGDYK

SEQ ID NO: 311 - protein, 12 aa, synthetic construct
YFFYSNIMNFFK

SEQ ID NO: 312 - protein, 11 aa, synthetic construct
TEITLANGEIR

SEQ ID NO: 313 - protein, 12 aa, synthetic construct
TEITLANGEIRK

SEQ ID NO: 314 - protein, 18 aa, synthetic construct
KRPLIETNGETGEIVWDK

SEQ ID NO: 315 - protein, 17 aa, synthetic construct
RPLIETNGETGEIVWDK

SEQ ID NO: 316 - protein, 19 aa, synthetic construct
RPLIETNGETGEIVWDKGR

SEQ ID NO: 317 - protein, 8 aa, synthetic construct
GRDFATVR

SEQ ID NO: 318 - protein, 11 aa, synthetic construct
KVLSMPQVNIV

SEQ ID NO: 319 - protein, 12 aa, synthetic construct
KVLSMPQVNIVK

SEQ ID NO: 320 - protein, 11 aa, synthetic construct
VLSMPQVNIVK

SEQ ID NO: 321 - protein, 11 aa, synthetic construct
KTEVQTGGFSK

SEQ ID NO: 322 - protein, 10 aa, synthetic construct
TEVQTGGFSK

SEQ ID NO: 323 - protein, 16 aa, synthetic construct
SKESGSVSSEQLAQFR

SEQ ID NO: 324 - protein, 16 aa, synthetic construct
LVQSGGGLVQAGGSLR

SEQ ID NO: 325 - protein, 11 aa, synthetic construct
TFSMYTMGWFR

SEQ ID NO: 326 - protein, 13 aa, synthetic construct
GLSPDIADSVNGR

SEQ ID NO: 327 - protein, 10 aa, synthetic construct
YSLFELENGR

SEQ ID NO: 328 - protein, 13 aa, synthetic construct
YVNFLYLASHYEK

SEQ ID NO: 329 - protein, 10 aa, synthetic construct
VILADANLDK

SEQ ID NO: 330 - protein, 19 aa, synthetic construct
EVLDATLIHQSITGLYETR

SEQ ID NO: 331 - protein, 13 aa, synthetic construct
IDLSQLGGDGSPK

SEQ ID NO: 332 - DNA, 21 bp, synthetic construct
  source 1..21: mol_type = other DNA
ccccaacccaggctggagtcc

SEQ ID NO: 333 - DNA, 21 bp, synthetic construct
  source 1..21: mol_type = other DNA
cctctttgccccaacccaggc

SEQ ID NO: 334 - DNA, 21 bp, synthetic construct
  source 1..21: mol_type = other DNA
tcccacagatatccagaaccc

SEQ ID NO: 335 - DNA, 21 bp, synthetic construct
  source 1..21: mol_type = other DNA
gagtctctcagctggtacacg

SEQ ID NO: 336 - DNA, 268 bp, synthetic construct
  source 1..268: mol_type = other DNA
ggtactggtggagtatttgatagtgtattaaccttatgtgtgacatgttctaatatagtcacattttcattatttttattataaggcctgctgaaaatgactgaatataaacttgtggtagttggagctagtggcgtaggcaagagtgccttgacgatacagctaattcagaatcattttgtggacgaatatgatccaacaatagaggtaaatgttgttttaatatgcatattactggtgcaggaccattctttgatacagataaagg

SEQ ID NO: 337 - DNA, 23 bp, synthetic construct
  source 1..23: mol_type = other DNA
ctggtggagtatttgatagtgta

SEQ ID NO: 338 - DNA, 23 bp, synthetic construct
  source 1..23: mol_type = other DNA
attcgtccacaaaatgattctga

SEQ ID NO: 339 - RNA, 56 nt, synthetic construct
  source 1..56: mol_type = other RNA
ttctaatacgactcactataggcttgtggtagttggagctaggttttagagctaga

SEQ ID NO: 340 - RNA, 20 nt, synthetic construct
  source 1..20: mol_type = other RNA
cttgtggtagttggagctag

SEQ ID NO: 341 - DNA, 21 bp, synthetic construct
  source 1..21: mol_type = other DNA
agtttgctttgctgggccttt

SEQ ID NO: 342 - DNA, 22 bp, synthetic construct
  source 1..22: mol_type = other DNA
tggcaatggataaggccgagac

SEQ ID NO: 343 - DNA, 15 bp, synthetic construct
  source 1..15: mol_type = other DNA
agccccgcccttgtc

SEQ ID NO: 344 - DNA, 33 bp, synthetic construct
  source 1..33: mol_type = other DNA
acatttgtttgagaatcaaaatcggtgaatagg

SEQ ID NO: 345 - DNA, 20 bp, synthetic construct
  source 1..20: mol_type = other DNA
acaaaactgtgctagacatg

SEQ ID NO: 346 - RNA, 20 nt, synthetic construct
  source 1..20: mol_type = other RNA
acaaaactgtgctagacatg

SEQ ID NO: 347 - DNA, 20 bp, synthetic construct
  source 1..20: mol_type = other DNA
aacaaatgtgtcacaaagta

SEQ ID NO: 348 - RNA, 20 nt, synthetic construct
  source 1..20: mol_type = other RNA
aacaaatgtgtcacaaagta

SEQ ID NO: 349 - DNA, 20 bp, synthetic construct
  source 1..20: mol_type = other DNA
tcagggttctggatatctgt

SEQ ID NO: 350 - RNA, 20 nt, synthetic construct
  source 1..20: mol_type = other RNA
tcagggttctggatatctgt

SEQ ID NO: 351 - DNA, 17 bp, synthetic construct
  source 1..17: mol_type = other DNA
gggttggggcaaagagg

SEQ ID NO: 352 - DNA, 19 bp, synthetic construct
  source 1..19: mol_type = other DNA
agagaggcagacagggagg

SEQ ID NO: 353 - DNA, 195 bp, synthetic construct
  source 1..195: mol_type = other DNA
atctaaatccagtgacaagtctgtctgcctattcaccgattttgattctcaaacaaatgtgtcacaaagtaaggattctgatgtgtatatcacagacaaaaatgctcaggctgctcttggctctcaacttattcccttcaattcaagtaacaggagggtcttcgactacaaggatcatgacggagactataagga

SEQ ID NO: 354 - protein, 45 aa, synthetic construct
TTGACCGCTGTGTGACGCAACACTCAAGCCTGTCCCTGAGTCCCA

SEQ ID NO: 355 - DNA, 47 bp, synthetic construct
  source 1..47: mol_type = other DNA
attgaccgctgtgtgacgcaacactcaatgcctgtccctgagtccca

SEQ ID NO: 356 - DNA, 58 bp, synthetic construct
  source 1..58: mol_type = other DNA
atctccgattgccccacgacgcagtggtcggagttactttgcctgtccctgagtccca